(19) United States
(12) Patent Application Publication
(10) Pub. No.: US 2026/0327299 A1
(43) Pub. Date: Oct. 22, 2026

(54) ISOFORM-SELECTIVE ANTI-TGF-BETA ANTIBODIES AND METHODS OF USE

(71) Applicant: Genentech, Inc., South San Francisco, CA (US)

(72) Inventors: Wei-Ching Liang, South San Francisco, CA (US); Joseph R. Arron, South San Francisco, CA (US); Daryle DePianto, South San Francisco, CA (US); Wendy Green Halpern, South San Francisco, CA (US); WeiYu Lin, South San Francisco, CA (US); Patrick J. Lupardus, South San Francisco, CA (US); Thirumalai Rajan Ramalingam, South San Francisco, CA (US); Dhaya Seshasayee, South San Francisco, CA (US); Tianhe Sun, South San Francisco, CA (US); Tulika Tyagi, South San Francisco, CA (US); Jia Wu, South San Francisco, CA (US); Yan Wu, South San Francisco, CA (US); Jian Ping Yin, South San Francisco, CA (US)

(73) Assignee: Genentech, Inc., South San Francisco, CA (US)

(21) Appl. No.: 19/728,204

(22) Filed: Jul. 1, 2026

Related U.S. Application Data
(62) Division of application No. 19/347,617, filed on Oct. 1, 2025.
Application No. 19/347,617 is a division of application No. 18/420,510, filed on Jan. 23, 2024, now abandoned.
Application No. 18/420,510 is a division of application No. 17/205,663, filed on Mar. 18, 2021, now Pat. No. 11,919,948.
(60) Provisional application No. 62/991,806, filed on Mar. 19, 2020.
(60) Provisional application No. 63/044,478, filed on Jun. 26, 2020.

(51) Int. Cl. C07K 16/22; A61K 39/00; A61K 39/395; A61P 11/00; C07K 16/46

(52) U.S. Cl. CPC C07K 16/22; A61P 11/00; C07K 16/464; A61K 39/39533; A61K 39/39566; A61K 2039/505; C07K 2317/24; C07K 2317/31; C07K 2317/41; C07K 2317/565; C07K 2317/76; C07K 2317/92

(57) ABSTRACT

The invention provides isoform-selective anti-TGFβ antibodies and methods of using the same. In particular, isoform-selective anti-TGFβ2, anti-TGFβ3, and anti-TGFβ2/3 monoclonal antibodies are provided, e.g., for the treatment of fibrosis and other TGFβ-related disorders.

### CROSS-REFERENCE TO RELATED APPLICATIONS

This application is a divisional of U.S. patent application Ser. No. 19/347,617, filed Oct. 1, 2025, which is a divisional of U.S. patent application Ser. No. 18/420,510, filed Jan. 23, 2024 (now abandoned), which is a divisional of U.S. patent application Ser. No. 17/205,663, filed Mar. 18, 2021, now U.S. Pat. No. 11,919,948, which claims the benefit of and priority to U.S. Provisional Application No. 63/044,478, filed Jun. 26, 2020, and U.S. Provisional Application No. 62/991,806, filed Mar. 19, 2020. The disclosures of each of the foregoing applications are incorporated herein by reference in their entireties.

### SEQUENCE LISTING

The instant application contains a Sequence Listing which has been submitted electronically in XML format and is hereby incorporated by reference in its entirety. Said XML copy, created on Jul. 1, 2026, is named “30006-030141-US5-SL.xml”, and is 384,934 bytes in size.

### FIELD OF THE INVENTION

The present invention relates to isoform-selective anti-TGFβ antibodies (e.g., monospecific anti-TGFβ2 and anti-TGFβ3 antibodies, and dual-specific, anti-TGFβ2/3 antibodies) and methods of using the same, e.g., for the treatment of TGFβ-related disorders.

### BACKGROUND

TGFβ comprises a pleiotropic set of three cytokines—TGFβ1, TGFβ2, and TGFβ3—that play critical roles in cell differentiation, tissue development, wound repair, immunoregulation, and, when dysregulated, tissue fibrosis. In the case of interstitial lung diseases (ILD) such as idiopathic pulmonary fibrosis (IPF), TGFβ activity is implicated in multiple aspects of disease pathogenesis. Genetic risk for IPF is conferred by mutations in genes expressed in lung epithelial cells that increase their susceptibility to injury and/or compromise their regenerative capacity. This epithelial stress or damage can activate innate immune cells such as alveolar macrophages to produce cytokines that activate mesenchymal cells to initiate a wound-healing response by proliferating, migrating, differentiating into myofibroblasts, and secreting extracellular matrix (ECM). TGFβ can contribute to many of these processes; in particular, it has been shown to promote apoptosis of lung epithelial cells while promoting activation, differentiation, and survival of myofibroblasts. Systemic sclerosis (SSc)/scleroderma is an autoimmune disease that begins with microvascular inflammation progressing to multi-organ connective tissue dysfunction involving tissues in skin, lung, heart, kidney, and intestine. TGFβ is involved in the dysregulation of vascular, connective tissue, and immune components in SSc (Lafyatis R. Nat Rev Rheumatol. 2014 December; 10(12):706-19).

TGFβ signaling also plays roles in cancer pathogenesis, particularly in peritumoral stroma and immune compartments, where it can inhibit productive anti-tumor immune responses both by promoting excessive ECM production that prevents T cell infiltration into tumor tissue and by promoting T regulatory cell differentiation and activation, which can suppress anti-tumor immunity. Taken together, these findings implicate TGFβ as a potential therapeutic target for fibrotic diseases and cancer. However, the multifarious homeostatic functions of TGFβ and complexity in context-dependent mechanisms of TGFβ activation have contributed to limitations in terms of both safety and efficacy to establish a favorable therapeutic index in interventional studies of TGFβ inhibitors in human fibrotic disorders. For example, pan-TGFβ inhibitors have been associated with undesirable safety signals. In particular, the small molecule ALK5 inhibitors AZ12601011 and AZ12799734 (Anderton et al. (2011) Toxicologic Pathology, 39: 916-924) caused microscopic heart valve lesions in rats, and the pan-TGFβ1D11 antibody (Lonning et al. (2011) Current Pharmaceutical Biotechnology, 12, 2176-2189) caused mice treated with 1D11 to develop histologic lesions, weight loss, nonneoplastic cystic epithelial hyperplasia and inflammation of the tongue and dental dysplasia and epithelial hyperplasia of the gingiva and esophagus. Further, CAT-192 (metelimumab), an antibody predominantly selective for TGFβ1, had a high serious adverse event rate with multiple gastric hemorrhages observed in a phase 1-2 study in SSc (Denton A&R 56:323 (2007)).

Thus, there remains a need in the art for safe and efficacious molecules that target TGFβ. The present invention provides such molecules and related uses.

### SUMMARY

The invention provides isoform-selective anti-TGFβ antibodies and methods of using the same.

In one aspect, an isolated anti-TGFβ3 antibody is provided, wherein the antibody comprises: (i) heavy chain CDRs comprising CDR-H1, -H2 and -H3, wherein CDR-H1 has the amino acid sequence of SEQ ID NO: 4, CDR-H2 has the amino acid sequence of one of SEQ ID NOs: 5, 34, 35, and 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9. In some aspects, the antibody selectively neutralizes TGFβ3.

In another aspect, an isolated anti-TGFβ3 antibody is provided, wherein the antibody selectively neutralizes TGFβ3, and wherein the antibody comprises one or more of the following features: (a) the anti-TGFβ3 antibody specifically binds to the beta6/beta7 hairpin region of TGFβ3; (b) binding of the anti-TGFβ3 antibody sterically blocks the ability of TGFBR2, but not TGFBR1, to bind TGFβ3; (c) binding of the anti-TGFβ3 antibody to TGFβ3 blocks TGFBR2 binding and inhibits the TGFBR1/TGFBR2 signaling receptors from binding to TGFβ3; (d) the anti-TGFβ3 antibody directly contacts amino acid residue R394 in the beta6/beta7 hairpin region of human TGFβ3; (e) the anti-TGFβ3 antibody directly contacts amino acid residue R394 in the beta6/beta7 hairpin region of human TGFβ3 and residue R394 of TGFβ3 makes an ionic salt bridge with the anti-TGFβ3 antibody in the heavy chain CDR2; (f) isoform selectivity of the anti-TGFβ3 antibody for TGFβ3 over TGFβ1 is achieved by direct contact by the antigen binding domain of the anti-TGFβ3 antibody with amino acid residues T387, L389, and T395 of TGFβ3 (human TGFβ3 numbering); (g) the isoform selectivity of the anti-TGFβ3 antibody for TGFβ3 over TGFβ2 is achieved by direct contact by the antigen binding domain of the anti-TGFβ3 antibody with amino acid residues R325, R394, and V398 of TGFβ3 (human TGFβ3 numbering); (h) the anti-TGFβ3 antibody has reduced toxicity relative to the pan-TGFβ antibody 1D11; (i) the anti-TGFβ3 antibody has reduced toxicity in rodents or cynomolgus monkeys relative to the pan-TGFβ antibody 1D11; (j) the anti-TGFβ3 antibody has reduced toxicity relative to the pan-TGFβ small molecule inhibitor galunisertib; (k) the anti-TGFβ3 antibody has reduced toxicity in rodents relative to the pan-TGFβ small molecule inhibitor galunisertib; (l) the anti-TGFβ3 antibody has reduced toxicity relative to the anti-TGFβ1 antibody CAT-192; (m) the anti-TGFβ3 antibody has reduced toxicity relative to an isoform selective anti-TGFβ2 antibody and/or anti-TGFβ2/3 antibody; (n) the anti-TGFβ3 antibody comprises: (i) heavy chain CDRs comprising CDR-H1, -H2 and -H3, wherein CDR-H1 has the amino acid sequence of SEQ ID NO: 4, CDR-H2 has the amino acid sequence of one of SEQ ID NOs: 5, 34, 35, and 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (o) the anti-TGFβ3 antibody comprises an antigen binding domain that directly contacts amino acid residues R325, K331, W332, H334, E335, T387, I388, L389, Y391, V392, G393, R394, P396, K397, and V398 on human TGFβ3; and (p) the anti-TGFβ3 antibody as in (o), wherein the antigen binding domain is within 15-8, 8, 8-5, 7-5, 6-5, or 5 angstroms of the TGFβ3 amino acid residues.

In another aspect, an isolated anti-TGFβ3 antibody is provided, wherein the antibody selectively neutralizes TGFβ3, and wherein the antibody comprises one or more of the following features: (a) the anti-TGFβ3 antibody specifically binds to the beta6/beta7 hairpin region of TGFβ3; (b) binding of the anti-TGFβ3 antibody sterically blocks the ability of TGFBR2, but not TGFBR1, to bind TGFβ3; (c) binding of the anti-TGFβ3 antibody to TGFβ3 blocks TGFBR2 binding and inhibits the TGFBR1/TGFBR2 signaling receptors from binding to TGFβ3; (d) the anti-TGFβ3 antibody directly contacts amino acid residue R394 in the beta6/beta7 hairpin region of human TGFβ3; (e) the anti-TGFβ3 antibody directly contacts amino acid residue R394 in the beta6/beta7 hairpin region of human TGFβ3 and residue R394 of TGFβ3 makes an ionic salt bridge with the anti-TGFβ3 antibody in the heavy chain CDR2; (f) isoform selectivity of the anti-TGFβ3 antibody for TGFβ3 over TGFβ1 is achieved by direct contact by the antigen binding domain of the anti-TGFβ3 antibody with amino acid residues T387, L389, and T395 of TGFβ3 (human TGFβ3 numbering); (g) the isoform selectivity of the anti-TGFβ3 antibody for TGFβ3 over TGFβ2 is achieved by direct contact by the antigen binding domain of the anti-TGFβ3 antibody with amino acid residues R325, R394, and V398 of TGFβ3 (human TGFβ3 numbering); (h) the anti-TGFβ3 antibody has reduced toxicity relative to the pan-TGFβ antibody 1D11; (i) the anti-TGFβ3 antibody has reduced toxicity in rodents or cynomolgus monkeys relative to the pan-TGFβ antibody 1D11; (j) the anti-TGFβ3 antibody has reduced toxicity relative to the pan-TGFβ small molecule inhibitor galunisertib; (k) the anti-TGFβ3 antibody has reduced toxicity in rodents relative to the pan-TGFβ small molecule inhibitor galunisertib; (l) the anti-TGFβ3 antibody has reduced toxicity relative to the anti-TGFβ1 antibody CAT-192; (m) the anti-TGFβ3 antibody has reduced toxicity relative to an isoform selective anti-TGFβ2 antibody and/or anti-TGFβ2/3 antibody; (n) the anti-TGFβ3 antibody comprises: (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 5, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (o) the anti-TGFβ3 antibody comprises an antigen binding domain that directly contacts amino acid residues R325, K331, W332, H334, E335, T387, I388, L389, Y391, V392, G393, R394, P396, K397, and V398 on human TGFβ3; and (p) the anti-TGFβ3 antibody as in (o), wherein the antigen binding domain is within 15-8, 8, 8-5, 7-5, 6-5, or 5 angstroms of the TGFβ3 amino acid residues. In some aspects, the anti-TGFβ3 antibody comprises a heavy chain variable region (VH) amino acid sequence having at least 95%, at least 96%, at least 97%, at least 98%, at least 99%, or 100% sequence identity to an amino acid sequence selected from the group consisting of SEQ ID NOs: 23, 37, and 42-50. In some aspects, the anti-TGFβ3 antibody comprises a VH amino acid sequence selected from the group consisting of SEQ ID NOs: 23, 37, and 42-50. In some aspects, the anti-TGFβ3 antibody comprises a complete heavy (H) chain amino acid sequence having at least 95%, at least 96%, at least 97%, at least 98%, at least 99%, or 100% sequence identity to an amino acid sequence selected from the group consisting of SEQ ID NOs: 29, 59, 64, and 65-72. In some aspects, the anti-TGFβ3 antibody comprises a complete heavy (H) chain amino acid sequence selected from the group consisting of SEQ ID NOs: 29, 59, 64, and 65-72. In further aspects, the anti-TGFβ3 antibody comprises a light chain variable region (VL) amino acid sequence having at least 95%, at least 96%, at least 97%, at least 98%, at least 99%, or 100% sequence identity an amino acid sequence selected from the group consisting of SEQ ID NOs: 22, 36, and 38-41. In yet other aspects, the anti-TGFβ3 antibody comprises a complete light (L) chain amino acid sequence having at least 95%, at least 96%, at least 97%, at least 98%, at least 99%, or 100% sequence identity to an amino acid sequence selected from the group consisting of SEQ ID NOs: 28, 58, and 60-63. In some aspects, the anti-TGFβ3 antibody comprises a complete L chain amino acid sequence selected from the group consisting of SEQ ID NOs: 28, 58, and 60-63. In some aspects, the anti-TGFβ3 antibody comprises a VH/VL pair, the VH/VL pair comprising amino acid sequences (respectively) selected from the group consisting of SEQ ID NOs: 23/22 (rat 2A10), SEQ ID NOs: 37/36 (v1), SEQ ID NOs: 37/38 (v1.1), SEQ ID NOs: 37/39 (v1.2), SEQ ID NOs: 37/40 (v1.3), SEQ ID NOs: 37/41 (v1.4), SEQ ID NOs: 42/36 (v1.5), SEQ ID NOs: 43/36 (v1.6), SEQ ID NOs:44/36 (v1.7), SEQ ID NOs: 45/36 (v2), SEQ ID NOs: 45/38 (v2.1), SEQ ID NOs: 45/39 (v2.2), SEQ ID NOs: 45/40 (v2.3), SEQ ID NOs: 45/41 (v2.4), SEQ ID NOs: 46/36 (v2.5), SEQ ID NOs: 47/36 (v2.6), SEQ ID NOs: 48/36 (v2.7), SEQ ID NOs: 49/36 (v2.8), and SEQ ID NOs: 50/36 (v2.9). In some aspects, the anti-TGFβ3 antibody comprises a complete H/L chain pair, the complete H/L chain pair comprising amino acid sequences (respectively) selected from the group consisting of SEQ ID NOs: 29/28 (rat 2A10), SEQ ID NOs: 59/58 (v1), SEQ ID NOs: 59/60 (v1.1), SEQ ID NOs: 59/61 (v1.2), SEQ ID NOs: 59/62 (v1.3), SEQ ID NOs: 59/63 (v1.4), SEQ ID NOs: 64/58 (v1.5), SEQ ID NOs: 65/58 (v1.6), SEQ ID NOs: 66/58 (v1.7), SEQ ID NOs: 67/58 (v2), SEQ ID NOs: 67/60 (v2.1), SEQ ID NOs: 67/61 (v2.2), SEQ ID NOs: 67/62 (v2.3), SEQ ID NOs: 67/63 (v2.4), SEQ ID NOs: 68/58 (v2.5), SEQ ID NOs: 69/58 (v2.6), SEQ ID NOs: 70/58 (v2.7), SEQ ID NOs: 71/58 (v2.8), SEQ ID NOs: 72/58 (v2.9). In some aspects, the anti-TGFβ3 antibody comprises a VL of SEQ ID NO: 22 comprising one or more framework modifications selected from the group consisting of: 4L or 4M, 38H or 38Q, 43A or 43Q, and 58V. In some aspects, the anti-TGFβ3 antibody VL comprises a set of framework modifications selected from the group consisting of: (i) 4L in FR1, 38H and 43Q in FR2, 58I in FR3 (h2A10.v1 and h2A10.v2); (ii) 4M in FR1 (h2A10.v1.1 and h2A10.v2.1); (iii) 38Q in FR2 (h2A10.v1.2 and h2A10.v2.2); (iv) 43A in FR2 (h2A10.v1.3 and h2A10.v2.3); (v) 58V in FR3 (h2A10.v1.4 and h2A10.v2.4); (vi) 38Q, 43A in FR2, 58V in FR3 (h2A10.v3 and h2A10.v4); (vii) 58V in FR3 (h2A10.v1.4 and h2A10.v2.4); and (vi) 38Q, 43A in FR2, 58V in FR3 (h2A10.v3 and h2A10.v4), wherein the mutations are relative to the rat 2A10 VL having SEQ ID NO: 22. In other aspects, the anti-TGFβ3 antibody comprises a VH of SEQ ID NO: 23 comprising one or more framework modifications selected from the group consisting of: 47L or 47W; 49A, 49S, or 49G; 73D or 73N; and 76N, 78D or 78L, 78A, or 78V. In some aspects, the anti-TGFβ3 antibody VH comprises a set of framework modifications selected from the group consisting of: (i) 47L, 49A in FR2, 78V in FR3 (h2A10.v1); (ii) 47L, 49A in FR2, 73D, 76S, 78V in FR3 (h2A10.v2); (iii) 47W in FR2 (h2A10.v1.5); (iv): 49G in FR2 (h2A10.v1.6); (v) 78A in FR3 (h2A10.v1.7); (vi) 47W in FR2 (h2A10.v2.5); (vii) 49S in FR2 (h2A10.v2.6); (viii) 73N in FR3 (h2A10.v2.7); (ix) 76N in FR3 (h2A10.v2.8); (x) 78L in FR3 (h2A10.v2.9); and (xi) 49S in FR2, 76N, 78L in FR3 (h2A10.v3 and h2A10.v4), wherein the mutations are relative to the rat 2A10 VH having SEQ ID NO: 23. In certain embodiments of the above anti-TGFβ3 antibodies, the VL of the anti-TGFβ3 antibody retains leucine (L) at position 4 in framework I and leucine (L) at position 47 in framework II (relative to the rat 2A10 VL having SEQ ID NO: 22). In some embodiments, the VH retains D at position 73 in framework III of VH from rat 2A10 (relative to the rat 2A10 VH having SEQ ID NO: 23).

In another aspect, an isolated anti-TGFβ3 antibody is provided, wherein the antibody selectively neutralizes TGFβ3, and wherein the antibody comprises one or more of the following features: (a) the anti-TGFβ3 antibody specifically binds to the beta6/beta7 hairpin region of TGFβ3; (b) binding of the anti-TGFβ3 antibody sterically blocks the ability of TGFBR2, but not TGFBR1, to bind TGFβ3; (c) binding of the anti-TGFβ3 antibody to TGFβ3 blocks TGFBR2 binding and inhibits the TGFBR1/TGFBR2 signaling receptors from binding to TGFβ3; (d) the anti-TGFβ3 antibody directly contacts amino acid residue R394 in the beta6/beta7 hairpin region of human TGFβ3; (e) the anti-TGFβ3 antibody directly contacts amino acid residue R394 in the beta6/beta7 hairpin region of human TGFβ3 and residue R394 of TGFβ3 makes an ionic salt bridge with the anti-TGFβ3 antibody in the heavy chain CDR2; (f) isoform selectivity of the anti-TGFβ3 antibody for TGFβ3 over TGFβ1 is achieved by direct contact by the antigen binding domain of the anti-TGFβ3 antibody with amino acid residues T387, L389, and T395 of TGFβ3 (human TGFβ3 numbering); (g) the isoform selectivity of the anti-TGFβ3 antibody for TGFβ3 over TGFβ2 is achieved by direct contact by the antigen binding domain of the anti-TGFβ3 antibody with amino acid residues R325, R394, and V398 of TGFβ3 (human TGFβ3 numbering); (h) the anti-TGFβ3 antibody has reduced toxicity relative to the pan-TGFβ antibody 1D11; (i) the anti-TGFβ3 antibody has reduced toxicity in rodents or cynomolgus monkeys relative to the pan-TGFβ antibody 1D11; (j) the anti-TGFβ3 antibody has reduced toxicity relative to the pan-TGFβ small molecule inhibitor galunisertib; (k) the anti-TGFβ3 antibody has reduced toxicity in rodents relative to the pan-TGFβ small molecule inhibitor galunisertib; (l) the anti-TGFβ3 antibody has reduced toxicity relative to the anti-TGFβ1 antibody CAT-192; (m) the anti-TGFβ3 antibody has reduced toxicity relative to an isoform selective anti-TGFβ2 antibody and/or anti-TGFβ2/3 antibody; (n) the anti-TGFβ3 antibody comprises: (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (o) the anti-TGFβ3 antibody comprises an antigen binding domain that directly contacts amino acid residues R325, K331, W332, H334, E335, T387, I388, L389, Y391, V392, G393, R394, P396, K397, and V398 on human TGFβ3; and (p) the anti-TGFβ3 antibody as in (o), wherein the antigen binding domain is within 15-8, 8, 8-5, 7-5, 6-5, or 5 angstroms of the TGFβ3 amino acid residues. In some aspects of this embodiment, the anti-TGFβ3 antibody comprises a VH amino acid sequence having at least 95%, at least 96%, at least 97%, at least 98%, at least 99%, or 100% sequence identity to the amino acid sequence of SEQ ID NO: 52. In some aspects, the anti-TGFβ3 antibody comprises the VH amino acid sequence of SEQ ID NO: 52. In some aspects, the anti-TGFβ3 antibody comprises a complete H chain amino acid sequence having at least 95%, at least 96%, at least 97%, at least 98%, at least 99%, or 100% sequence identity to the amino acid sequence of SEQ ID NO: 74. In some aspects, the anti-TGFβ3 antibody comprises the complete H chain amino acid sequence of SEQ ID NO: 74. In some aspects, the anti-TGFβ3 comprises a VL amino acid sequence having at least 95%, at least 96%, at least 97%, at least 98%, at least 99%, or 100% sequence identity to an amino acid sequence selected from the group consisting of SEQ ID NOs: 22, 36, and 38-41. In some aspects, the anti-TGFβ3 antibody comprises a VL amino acid sequence selected from the group consisting of SEQ ID NOs: 22, 36, and 38-41. In some aspects, the anti-TGFβ3 antibody comprises a complete L chain amino acid sequence having at least 95%, at least 96%, at least 97%, at least 98%, at least 99%, or 100% sequence identity to an amino acid sequence selected from the group consisting of SEQ ID NOs: 28, 58, and 60-63. In some aspects, the anti-TGFβ3 comprises a complete L chain amino acid sequence selected from the group consisting of SEQ ID NOs: 28, 58, and 60-63. In further aspects, the anti-TGFβ3 antibody comprises a VH/VL pair comprising the amino acid sequences (respectively) of SEQ ID NOs: 52/36. In some aspects, the anti-TGFβ3 antibody comprises a complete H/L chain pair, the H/L chain pair comprising the amino acid sequences (respectively) of SEQ ID NOs: 74/58.

In another aspect, an isolated anti-TGFβ3 antibody is provided, wherein the antibody selectively neutralizes TGFβ3, and wherein the antibody comprises one or more of the following features: (a) the anti-TGFβ3 antibody specifically binds to the beta6/beta7 hairpin region of TGFβ3; (b) binding of the anti-TGFβ3 antibody sterically blocks the ability of TGFBR2, but not TGFBR1, to bind TGFβ3; (c) binding of the anti-TGFβ3 antibody to TGFβ3 blocks TGFBR2 binding and inhibits the TGFBR1/TGFBR2 signaling receptors from binding to TGFβ3; (d) the anti-TGFβ3 antibody directly contacts amino acid residue R394 in the beta6/beta7 hairpin region of human TGFβ3; (e) the anti-TGFβ3 antibody directly contacts amino acid residue R394 in the beta6/beta7 hairpin region of human TGFβ3 and residue R394 of TGFβ3 makes an ionic salt bridge with the anti-TGFβ3 antibody in the heavy chain CDR2; (f) isoform selectivity of the anti-TGFβ3 antibody for TGFβ3 over TGFβ1 is achieved by direct contact by the antigen binding domain of the anti-TGFβ3 antibody with amino acid residues T387, L389, and T395 of TGFβ3 (human TGFβ3 numbering); (g) the isoform selectivity of the anti-TGFβ3 antibody for TGFβ3 over TGFβ2 is achieved by direct contact by the antigen binding domain of the anti-TGFβ3 antibody with amino acid residues R325, R394, and V398 of TGFβ3 (human TGFβ3 numbering); (h) the anti-TGFβ3 antibody has reduced toxicity relative to the pan-TGFβ antibody 1D11; (i) the anti-TGFβ3 antibody has reduced toxicity in rodents or cynomolgus monkeys relative to the pan-TGFβ antibody 1D11; (j) the anti-TGFβ3 antibody has reduced toxicity relative to the pan-TGFβ small molecule inhibitor galunisertib; (k) the anti-TGFβ3 antibody has reduced toxicity in rodents relative to the pan-TGFβ small molecule inhibitor galunisertib; (l) the anti-TGFβ3 antibody has reduced toxicity relative to the anti-TGFβ1 antibody CAT-192; (m) the anti-TGFβ3 antibody has reduced toxicity relative to an isoform selective anti-TGFβ2 antibody and/or anti-TGFβ2/3 antibody; (n) the anti-TGFβ3 antibody comprises: (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 34, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (o) the anti-TGFβ3 antibody comprises an antigen binding domain that directly contacts amino acid residues R325, K331, W332, H334, E335, T387, I388, L389, Y391, V392, G393, R394, P396, K397, and V398 on human TGFβ3; and (p) the anti-TGFβ3 antibody as in (o), wherein the antigen binding domain is within 15-8, 8, 8-5, 7-5, 6-5, or 5 angstroms of the TGFβ3 amino acid residues. In some aspects, the anti-TGFβ3 antibody comprises a VH amino acid sequence having at least 95%, at least 96%, at least 97%, at least 98%, at least 99%, or 100% sequence identity to the amino acid sequence of SEQ ID NO: 51 or 55. In some aspects, the anti-TGFβ3 antibody comprises the VH amino acid sequence of SEQ ID NO: 51 or 55. In some aspects, the anti-TGFβ3 antibody comprises a complete H chain amino acid sequence having at least 95%, at least 96%, at least 97%, at least 98%, at least 99%, or 100% sequence identity to the amino acid sequence of SEQ ID NO: 73 or 77. In some aspects, the anti-TGFβ3 antibody comprises the complete H chain amino acid sequence of SEQ ID NO: 73. In some aspects, the anti-TGFβ3 antibody comprises the complete H chain amino acid sequence of SEQ ID NO: 77. In some aspects, the anti-TGFβ3 antibody comprises a VL amino acid sequence having at least 95%, at least 96%, at least 97%, at least 98%, at least 99%, or 100% sequence identity to an amino acid sequence selected from the group consisting of SEQ ID NOs: 22, 36, and 38-41. In some aspects, the anti-TGFβ3 antibody comprises a VL amino acid sequence selected from the group consisting of SEQ ID NOs: 22, 36, and 38-41. In some aspects, the anti-TGFβ3 antibody comprises a complete L chain amino acid sequence having at least 95%, at least 96%, at least 97%, at least 98%, at least 99%, or 100% sequence identity to an amino acid sequence selected from the group consisting of SEQ ID NOs: 28, 58, and 60-63. In some aspects, the anti-TGFβ3 antibody comprises a complete L chain amino acid sequence selected from the group consisting of SEQ ID NOs: 28, 58, and 60-63. In some aspects, the anti-TGFβ3 antibody comprises a VH/VL pair, the VH/VL pair comprising the amino acid sequences (respectively) of SEQ ID NOs: 51/36 or SEQ ID NOs: 55/54. In some aspects, the anti-TGFβ3 antibody comprises a complete H/L chain pair, the H/L chain pair comprising the amino acid sequences (respectively) of SEQ ID NOs: 73/58 or SEQ ID NOs: 77/76.

In another aspect, an isolated anti-TGFβ3 antibody is provided, wherein the antibody selectively neutralizes TGFβ3, and wherein the antibody comprises one or more of the following features: (a) the anti-TGFβ3 antibody specifically binds to the beta6/beta7 hairpin region of TGFβ3; (b) binding of the anti-TGFβ3 antibody sterically blocks the ability of TGFBR2, but not TGFBR1, to bind TGFβ3; (c) binding of the anti-TGFβ3 antibody to TGFβ3 blocks TGFBR2 binding and inhibits the TGFBR1/TGFBR2 signaling receptors from binding to TGFβ3; (d) the anti-TGFβ3 antibody directly contacts amino acid residue R394 in the beta6/beta7 hairpin region of human TGFβ3; (e) the anti-TGFβ3 antibody directly contacts amino acid residue R394 in the beta6/beta7 hairpin region of human TGFβ3 and residue R394 of TGFβ3 makes an ionic salt bridge with the anti-TGFβ3 antibody in the heavy chain CDR2; (f) isoform selectivity of the anti-TGFβ3 antibody for TGFβ3 over TGFβ1 is achieved by direct contact by the antigen binding domain of the anti-TGFβ3 antibody with amino acid residues T387, L389, and T395 of TGFβ3 (human TGFβ3 numbering); (g) the isoform selectivity of the anti-TGFβ3 antibody for TGFβ3 over TGFβ2 is achieved by direct contact by the antigen binding domain of the anti-TGFβ3 antibody with amino acid residues R325, R394, and V398 of TGFβ3 (human TGFβ3 numbering); (h) the anti-TGFβ3 antibody has reduced toxicity relative to the pan-TGFβ antibody 1D11; (i) the anti-TGFβ3 antibody has reduced toxicity in rodents or cynomolgus monkeys relative to the pan-TGFβantibody 1D11; (j) the anti-TGFβ3 antibody has reduced toxicity relative to the pan-TGFβ small molecule inhibitor galunisertib; (k) the anti-TGFβ3 antibody has reduced toxicity in rodents relative to the pan-TGFβ small molecule inhibitor galunisertib; (l) the anti-TGFβ3 antibody has reduced toxicity relative to the anti-TGFβ1 antibody CAT-192; (m) the anti-TGFβ3 antibody has reduced toxicity relative to an isoform selective anti-TGFβ2 antibody and/or anti-TGFβ2/3 antibody; (n) the anti-TGFβ3 antibody comprises: (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 35, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (o) the anti-TGFβ3 antibody comprises an antigen binding domain that directly contacts amino acid residues R325, K331, W332, H334, E335, T387, I388, L389, Y391, V392, G393, R394, P396, K397, and V398 on human TGFβ3; and (p) the anti-TGFβ3 antibody as in (o), wherein the antigen binding domain is within 15-8, 8, 8-5, 7-5, 6-5, or 5 angstroms of the TGFβ3 amino acid residues. In some aspects, the anti-TGFβ3 antibody comprises a VH amino acid sequence having at least 95%, at least 96%, at least 97%, at least 98%, at least 99%, or 100% sequence identity to the amino acid sequence of SEQ ID NO: 53 or 57. In some aspects, the anti-TGFβ3 antibody comprises the VH amino acid sequence of SEQ ID NO: 53. In some aspects, the anti-TGFβ3 antibody comprises the VH amino acid sequence of SEQ ID NO: 57. In some aspects, the anti-TGFβ3 antibody comprises a complete H chain amino acid sequence having at least 95%, at least 96%, at least 97%, at least 98%, at least 99%, or 100% sequence identity to the amino acid sequence of SEQ ID NO: 75 or 79. In some aspects, the anti-TGFβ3 antibody comprises the complete H chain amino acid sequence of SEQ ID NO: 75. In some aspects, the anti-TGFβ3 antibody comprises a complete H chain amino acid sequence of SEQ ID NO: 79. In some aspects, the anti-TGFβ3 antibody comprises a VL amino acid sequence having at least 95%, at least 96%, at least 97%, at least 98%, at least 99%, or 100% sequence identity to an amino acid sequence selected from the group consisting of SEQ ID NOs: 22, 36, and 38-41. In some aspects, the anti-TGFβ3 antibody comprises a VL amino acid sequence selected from the group consisting of SEQ ID NOs: 22, 36, and 38-41. In some aspects, the anti-TGFβ3 antibody comprises a complete L chain amino acid sequence having at least 95%, at least 96%, at least 97%, at least 98%, at least 99%, or 100% sequence identity to the amino acid sequence selected from the group consisting of SEQ ID NOs: 28, 58, and 60-63. In some aspects, the anti-TGFβ3 antibody comprises a complete L chain amino acid sequence selected from the group consisting of SEQ ID NOs: 28, 58, and 60-63. In some aspects, the anti-TGFβ3 antibody comprises a VH/VL pair comprising the amino acid sequences (respectively) of SEQ ID NOs: 53/36 or SEQ ID NOs: 57/56. In some aspects, the anti-TGFβ3 antibody comprises a complete H/L chain pair, the H/L chain pair comprising the amino acid sequences (respectively) of SEQ ID NOs: 75/58 or SEQ ID NOs: 79/78.

In another aspect, an anti-TGFβ3 antibody is provided, the antibody comprising: (a) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 35, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (b) a VH/VL pair, the VH of the VH/VL pair comprising the amino acid sequence of SEQ ID NO: 57, and the VL of the VH/VL pair comprising the amino acid sequence of SEQ ID NO: 56; or (c) a complete H/L chain pair, the H chain of the H/L chain pair comprising the amino acid sequence of SEQ ID NO: 79 and the L chain of the H/L chain pair comprising the amino acid sequence of SEQ ID NO: 78.

In another aspect, an isolated anti-TGFβ2/3 antibody is provided, wherein the antibody selectively neutralizes TGFβ2 and TGFβ3, and wherein the antibody comprises one or more of the following features: (a) selectivity of the anti-TGFβ2/3 antibody for TGFβ2 and TGFβ3 over human TGFβ1, with respect to selective neutralization, is achieved by direct contact of the antibody's antigen binding domain with amino acid residue E373 TGFβ2 or TGFβ3 (human TGFβ2 numbering); (b) the anti-TGFβ2/3 antibody neutralizes TGFβ2 and/or TGFβ3 via an allosteric mechanism; (c) the anti-TGFβ2/3 antibody induces a conformational change in TGFβ2 and/or TGFβ3 homodimer; (d) the anti-TGFβ2/3 antibody induces a conformational change in TGFβ2 and/or TGFβ3 homodimer, wherein the conformational change comprises the two monomers pinching together by several degrees; (e) the anti-TGFβ2/3 antibody is a divalent antibody or a monovalent antibody; (f) the anti-TGFβ2/3 antibody comprises (i) heavy chain CDRs comprising CDR-H1, -H2 and -H3, wherein CDR-H1 has the amino acid sequence of SEQ ID NO: 10, CDR-H2 has the amino acid sequence of SEQ ID NO: 11, and CDR-H3 has the amino acid sequence of SEQ ID NO: 12; and (ii) light chain CDRs comprising CDR-L1, -L2 and -L3, wherein CDR-L1 has the amino acid sequence of SEQ ID NO: 13; CDR-L2 has the amino acid sequence of SEQ ID NO: 14, and CDR-L3 has the amino acid sequence of SEQ ID NO: 15; (g) the anti-TGFβ2/3 antibody specifically binds to TGFβ2 homodimer, the TGFβ2 homodimer having a first and a second TGFβ2 monomer, and wherein the anti-TGFβ2/3 antibody comprises an antigen binding domain that directly contacts (i) amino acid residues V313, Q314, D315, R320, L322, Y323, R328, D329, F345, and A347 of the first TGFβ2 monomer, and (ii) amino acid residues N368, T369, I370, N371, P372, E373, A374, S375, A376, and S377 of the second TGFβ2 monomer (human TGFβ2 numbering); (h) the anti-TGFβ2/3 antibody as in (g), wherein the antigen binding domain is within 5 angstroms of the TGFβ2 and/or TGFβ3 amino acid residues; (i) wherein the anti-TGFβ2/3 antibody specifically binds to the same epitope on TGFβ3 as in (g); and (j) the anti-TGFβ2/3 antibody does not neutralize TGFβ2 and/or TGFβ3 in monovalent form. In one aspect, the anti-TGFβ2/3 antibody comprises a VH amino acid sequence having at least 95%, at least 96%, at least 97%, at least 98%, at least 99%, or 100% sequence identity to an amino acid sequence selected from the group consisting of SEQ ID NOs: 27, 81, 83, 86-88, 93-100, and 102-105. In one aspect, the anti-TGFβ2/3 antibody comprises a VH amino acid sequence selected from the group consisting of SEQ ID NOs: 27, 81, 83, 86-88, 93-100, and 102-105. In one aspect, the anti-TGFβ2/3 antibody comprises a complete H chain amino acid sequence having at least 95%, at least 96%, at least 97%, at least 98%, at least 99%, or 100% sequence identity to the amino acid sequence selected from the group consisting of SEQ ID NOs: 33, 107, 109, 112-114, and 119-130. In one aspect, the anti-TGFβ2/3 comprises a VL amino acid sequence having at least 95%, at least 96%, at least 97%, at least 98%, at least 99%, or 100% sequence identity to the amino acid sequence selected from the group consisting of SEQ ID NOs: 26, 80, 82, 84, 85, 89-92, and 101. In one aspect, the anti-TGFβ2/3 antibody comprises a VL amino acid sequence selected from the group consisting of SEQ ID NOs: 26, 80, 82, 84, 85, 89-92, and 101. In one aspect, the anti-TGFβ2/3 antibody comprises a complete L chain amino acid sequence having at least 95%, at least 96%, at least 97%, at least 98%, at least 99%, or 100% sequence identity to an amino acid sequence selected from the group consisting of SEQ ID NOs: 32, 106, 108, 110, 111, 115-118, and 186. In one aspect, the anti-TGFβ2/3 antibody comprises a complete L chain amino acid sequence selected from the group consisting of SEQ ID NOs: 32, 106, 108, 110, 111, 115-118, and 186. In one aspect, the anti-TGFβ2/3 antibody comprises a VH/VL pair, the VH/VL pair comprising amino acid sequences (respectively) selected from the group consisting of SEQ ID NOs: 27/26 (rabbit 4A11), SEQ ID NOs: 81/80 (v1), SEQ ID NOs: 81/82 (v2), SEQ ID NOs: 83/80 (v3), SEQ ID NOs: 83/82 (v4), SEQ ID NOs: 81/84 (v5), SEQ ID NOs: 81/85 (v6), SEQ ID NOs: 83/84 (v7), SEQ ID NOs: 86/84 (v7/1), SEQ ID NOs: 87/84 (v7.2), SEQ ID NOs: 88/84 (v7.3), SEQ ID NOs: 83/89 (v7.4), SEQ ID NOs: 83/90 (v7.5), SEQ ID NOs: 83/91 (v7.6), SEQ ID NOs: 83/92 (v7.7), SEQ ID NOs: 93/84 (v7.8), SEQ ID NOs: 94/84 (v7.9), SEQ ID NOs: 95/84 (v7.10), SEQ ID NOs: 96/84 (v7.11), SEQ ID NOs: 97/84 (v7.12), SEQ ID NOs: 98/84 (v7.13), SEQ ID NOs: 99/84 (v7.14), SEQ ID NOs: 100/84 (v7.15), SEQ ID NOs: 102/101 (v7.16), SEQ ID NOs: 103/101 (v7.17), SEQ ID NOs: 104/101 (v7.18), SEQ ID NOs: 105/101 (v7.19), and SEQ ID NOs: 83/85 (v8). In one aspect, the anti-TGFβ2/3 antibody comprises a complete H/L chain pair, the complete H/L chain pair comprising amino acid sequences (respectively) selected from the group consisting of SEQ ID NOs: 32/33 (rabbit 4A11), SEQ ID NOs: 107/106 (v1), SEQ ID NOs: 107/108 (v2), SEQ ID NOs: 109/106 (v3), SEQ ID NOs: 109/108 (v4), SEQ ID NOs: 107/110 (v5), SEQ ID NOs: 107/111 (v6), SEQ ID NOs: 109/110 (v7), SEQ ID NOs: 112/110 (v7.1), SEQ ID NOs: 113/110 (v7.2), SEQ ID NOs: 114/110 (v7.3), SEQ ID NOs: 114/115 (v7.4), SEQ ID NOs: 114/116 (v7.5), SEQ ID NOs: 114/117 (v7.6), SEQ ID NOs: 114/118 (v7.7), SEQ ID NOs: 119/110 (v7.8), SEQ ID NOs:120/110 (v7.9), SEQ ID NOs: 121/110 (v7.10), SEQ ID NOs: 122/110 (v7.11), SEQ ID NOs: 123/110 (v7.12), SEQ ID NOs: 124/110 (v7.13), SEQ ID NOs: 125/110 (v7.14), SEQ ID NOs: 126/110 (v7.15), SEQ ID NOs: 127/186 (v7.16), SEQ ID NOs: 128/186 (v7.17), SEQ ID NOs: 129/186 (v7.18), SEQ ID NOs: 130/186 (v7.19), and SEQ ID NOs: 114/111 (v8). In one aspect, the anti-TGFβ2/3 antibody comprises a VL comprising the amino acid sequence of SEQ ID NO: 26 comprising one or more framework modifications selected from the group consisting of: 2A or 2I, 4L, 36F or 36Y, 43P or 43A, and 58V or 58I. In one aspect, the anti-TGFβ2/3 antibody VL comprises a set of framework modifications selected from the group consisting of: (i) 2A and 4L in FR1 and 36F in FR2 (h4A11.v1 and h4A11.v3); (ii) 2A and 4L in FR1 and 36F and 43P in FR2 (h4A11.v2 and h4A11.v4); (iii) 2A in FR1, 36F and 43P in FR2 and 58V in FR3 (h4A11.v5 and h4A11.v7); (iv) 2A and 4L in FR1 and 36F in FR2 (h4A11.v6 and h4A11.v8); (v) 2I in FR1 (h4A11.v7.4); (vi) 36Y in FR2 (h4A11.v7.5); (vii) 43A in FR2 (h4A11.v7.6); (viii) 58I in FR3 (h4A11.v7.7); and (ix) 2I in FR1, 43A in FR2, 58I in FR3 (h4A11.v7.16-19), wherein the mutations are relative to the VL comprising the amino acid sequence of SEQ ID NO: 26. In one aspect, the anti-TGFβ2/3 antibody comprises a VH comprising the amino acid sequence of SEQ ID NO: 27 comprising one or more framework modifications selected from the group consisting of: deletion of 1E, 2Q or 2V, 24V, 37V or 37I, 48I, 49G, 67F or 67V, 71K or 71V, 73S or 73T, deletion of 75K and 76N, 78V or 78F, 91F or 91Y, 105P or 105Q. In one aspect, the anti-TGFβ2/3 antibody VL comprises a set of framework modifications selected from the group consisting of: (i) 2Q and 24V in FR1, 48I and 49G in FR2, 71K, 73S, 78V and 91F in FR3 and 105P in FR4 (h4A11.v, h4A11.v2, h4A11.v5, h4A11.v6); (ii) 2Q in FR1, 37V in FR2, 67F, 71K, 73S, 78V and 91F in FR3 and 105P in FR4 (h4A11.v3, h4A11.v4, h4A11.v7, h4A11.v8); (iii) delete 1E in FR1 (h4A11.v7.1); (iv) delete 75K and 76N in FR3 (h4A11.v7.2); (v) delete 1E in FR1 and 75K76N in FR3 (h4A11.v7.3); (vi) 2V in FR1 (h4A11.v7.8); (vi) 37I in FR2 (h4A11.v7.9); (vii) 67V in FR3 (h4A11.v7.10); (viii) 71V in FR3 (h4A11.v7.11); (ix) 73T in FR3 (h4A11.v7.12); (x) 78F in FR3 (h4A11.v7.13); (xi) 91Y in FR3 (h4A11.v7.14); (xii) 105Q in FR4 (h4A11.v7.15); (xiii) 2V in FR1, 37I in FR2, 67V, 73T, 78F in FR3, 105Q in FR4 ((h4A11.v7.16); (xiv) 2V in FR1, 37I in FR2, 67V, 73T, 91Y in FR3, 105Q in FR4 (h4A11.v7.17); (xv) 2V in FR1, 37I in FR2, 67V, 73T in FR3, 105Q in FR4 (h4A11.v7.18); and (xvi) 2V in FR1, 37I in FR2, 67V, 73T, deletion of 75K and 76N in FR3, 105Q in FR4 (h4A11.v7.19), wherein the mutations are relative to the VH comprising the amino acid sequence of SEQ ID NO: 27.

In another aspect, an isolated anti-TGFβ2 antibody is provided, wherein the antibody comprises: (a) heavy chain CDRs comprising CDR-H1, -H2 and -H3, wherein CDR-H1 has the amino acid sequence of SEQ ID NO: 16, CDR-H2 has the amino acid sequence of SEQ ID NO: 17, and CDR-H3 has the amino acid sequence of SEQ ID NO: 18; and (b) light chain CDRs comprising CDR-L1, -L2 and -L3, wherein CDR-L1 has the amino acid sequence of SEQ ID NO: 19; CDR-L2 has the amino acid sequence of SEQ ID NO: 20, and CDR-L3 has the amino acid sequence of SEQ ID NO: 21. In one aspect, the anti-TGFβ2 antibody selectively neutralizes TGFβ2. In some aspects, the anti-TGFβ2 antibody: (a) has reduced toxicity relative to the pan-TGFβ antibody 1D11; (b) has reduced toxicity in rodents relative to the pan-TGFβ antibody 1D11; (c) has reduced toxicity relative to the pan-TGFβ small molecule inhibitor galunisertib; and/or (d) has reduced toxicity in rodents relative to the pan-TGFβ small molecule inhibitor galunisertib. In one aspect, the anti-TGFβ2 antibody comprises a VH amino acid sequence having at least 95%, at least 96%, at least 97%, at least 98%, at least 99%, or 100% sequence identity to an amino acid sequence selected from the group consisting of SEQ ID NOs: 25, 132, and 138-142. In one aspect, the anti-TGFβ2 antibody comprises a VH amino acid sequence selected from the group consisting of SEQ ID NOs: 25, 132, and 138-142. In one aspect, the anti-TGFβ2 antibody comprises a complete H chain amino acid sequence having at least 95%, at least 96%, at least 97%, at least 98%, at least 99%, or 100% sequence identity to the amino acid sequence selected from the group consisting of SEQ ID NOs: 31, 146, and 152-156. In one aspect, the anti-TGFβ2 antibody comprises a complete H chain amino acid sequence selected from the group consisting of SEQ ID NOs: 31, 146, and 152-156. In one aspect, the anti-TGFβ2 antibody comprises a VL amino acid sequence having at least 95%, at least 96%, at least 97%, at least 98%, at least 99%, or 100% sequence identity to the amino acid sequence selected from the group consisting of SEQ ID NOs: 24, 131, 133-137, 143, and 144. In another aspect, the anti-TGFβ2 antibody comprises a VL amino acid sequence selected from the group consisting of SEQ ID NOs: 24, 131, 133-137, 143, and 144. In one aspect, the anti-TGFβ2 antibody comprises a complete L chain amino acid sequence having at least 95%, at least 96%, at least 97%, at least 98%, at least 99%, or 100% sequence identity to an amino acid sequence selected from the group consisting of SEQ ID NOs: 30, 145, 147-151, 157, and 158. In one aspect, the anti-TGFβ2 antibody comprises a complete L chain amino acid sequence selected from the group consisting of SEQ ID NOs: 30, 145, 147-151, 157, and 158. In one aspect, the anti-TGFβ2 antibody comprises a VH/VL pair, the VH/VL pair comprising amino acid sequences selected from the group consisting of SEQ ID NOs: 25/24 (rabbit 6F12), SEQ ID NOs: 132/131 (v1), SEQ ID NOs: 132/133 (v1.1), SEQ ID NOs: 132/134 (v1.2), SEQ ID NOs: 132/135 (v1.3), SEQ ID NOs: 132/136 (v1.4), SEQ ID NOs: 132/137 (v1.5), SEQ ID NOs: 138/131 (v1.6), SEQ ID NOs: 139/131 (v1.7), SEQ ID NOs: 140/131 (v1.8), SEQ ID NOs: 141/131 (v1.9), SEQ ID NOs: 142/131 (v2), SEQ ID NOs: 132/143 (v3), and SEQ ID NOs: 142/144 (v4). In one aspect, the anti-TGFβ2 comprises a complete H/L chain pair, the complete H/L chain pair comprising amino acid sequences selected from the group consisting of SEQ ID NOs: 31/30 (rabbit 6F12), SEQ ID NOs: 146/145 (v1), SEQ ID NOs: 146/147 (v1.1), SEQ ID NOs: 146/148 (v1.2), SEQ ID NOs: 146/149 (v1.3), SEQ ID NOs: 146/150 (v1.4), SEQ ID NOs: 146/151 (v1.5), SEQ ID NOs: 152/145 (v1.6), SEQ ID NOs: 153/145 (v1.7), SEQ ID NOs: 154/145 (v1.8), SEQ ID NOs: 155/145 (v1.9), SEQ ID NOs: 156/145 (v2), SEQ ID NOs: 146/157 (v3), and SEQ ID NOs: 156/158 (v4). In one aspect, the anti-TGFβ2 antibody comprises a VL comprising the amino acid sequence of SEQ ID NO: 24 comprising one or more framework mutations selected from the group consisting of 43S or 43A, 66G, 69T, 71F, and 87Y. In one aspect, the anti-TGFβ2 antibody VL comprises a set of framework mutations selected from the group consisting of: (i) 43S in FR2 and 66E, 69P, 71Y and 87F in FR3 (h6F12.v1 and h6F12.v2); (ii) 43S in FR2 and 58V, 66E, 69P, 71Y and 87F in FR3 (h6F12.v3 and h6F12.v4); (iii) 43A in FR2 (h6F12.v1.1); (iv) 66G in FR3 (h6F12.v1.2); (v) 69T in FR3 (h6F12.v1.3); (vi) 71F in FR3 (h6F12.v1.4); and (vii) 87Y in FR3 (h6F12.v1.5), wherein the mutations are relative to the VL comprising the amino acid sequence of SEQ ID NO: 24. In one aspect, the anti-TGFβ2 antibody comprises a VH comprising the amino acid sequence of SEQ ID NO: 25 comprising one or more framework mutations selected from the group consisting of 37V or 37I, 48M or 48L, 49G or 49A, 67L, 71K and 78V, and 105P or 105R. In one aspect, the anti-TGFβ2 antibody VH comprises a set of framework mutations selected from the group consisting of (i) 37V, 48M and 49G in FR2 and 105P in FR4 (h6F12.v1 and h6F12.v3); (ii) 37V and 48M in FR2, 67L, 71K and 78V in FR3 and 105P in FR4 (h6F12.v2 and h6F12.v4); (iii) 37I in FR2 (h6F12.v1.6); (iv) 48L in FR2 (h6F12.v1.7); (v) 49A in FR2 (h6F12.v1.8); (vi) 105R in FR4 (h6F12.v1.9); (vii) 37V, 48M and 49G in FR2 and 105P in FR4 (h6F12.v1 and h6F12.v3); (viii) 37V and 48M in FR2, 67L, 71K and 78V in FR3 and 105P in FR4 (6F12.v2 and h6F12.v4); (ix) 37I in FR2 (h6F12.v1.6); (x) 48L in FR2 (h6F12.v1.7); (xi): 49A in FR2 (h6F12.v1.8); and (xii) 105R in FR4 (h6F12.v1.9), wherein the mutations are relative to the VH comprising the amino acid sequence of SEQ ID NO: 25.

In certain embodiments of any of the above aspects, the anti-TGFβ3 antibody and/or the anti-TGFβ2/3 antibody specifically binds to human TGFβ3. In some embodiments of any of the above aspects, the anti-TGFβ3 antibody specifically binds to both the immature and mature forms of TGFβ3. In some embodiments of any of the above aspects, the anti-TGFβ2/3 antibody and/or the anti-TGFβ2 specifically binds to human TGFβ2. In some embodiments of any of the above aspects, the antibody is a monoclonal antibody. In some embodiments of any of the above aspects, the antibody is a human, humanized, or chimeric antibody. In some embodiments of any of the above aspects, the antibody is an antibody fragment. In some embodiments of any of the above aspects, the antibody comprises a human Fc region that is an IgG1 or IgG4 isotype. In some embodiments of any of the above aspects, the antibody comprises a human Fc region that is an IgG1 isotype. In some embodiments of any of the above aspects, the Fc region of the antibody is modified to remove effector function. In some aspects, the Fc region comprises a modification to remove the glycosylation site at amino acid residue position N297 (EU numbering as in Kabat). In some aspects, the modification is a mutation selected from N297G or N297A. In some aspects, the modification is the mutation N297G. In some embodiments of any of the above aspects, the antibody has a Cmax of about 230-260 g/ml and/or a half life (t_{1/2}) of about 15 to 16 days.

Also provided are isolated nucleic acids encoding the antibody according to any of the above aspects and embodiments, and host cells comprising the nucleic acids. In some aspects, a method of producing an antibody is provided. The method can include culturing the host cell provided herein so that the antibody is produced. In some aspects, the method further comprises recovering the antibody from the host cell. Also provided is an antibody produced by the method of producing the antibody.

In another aspect, an immunoconjugate comprising an antibody, such as any of the antibodies as described above, and a cytotoxic agent is provided. In some embodiments, the antibody is an anti-TGFβ3 antibody comprising: (a1) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 5, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (a2) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 34, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (a3) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 35, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; or (a4) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; or (b) a VH/VL pair, the VH of the VH/VL pair comprising the amino acid sequence of SEQ ID NO: 57, and the VL of the VH/VL pair comprising the amino acid sequence of SEQ ID NO: 56; or (c) a complete H/L chain pair, the H chain of the H/L chain pair comprising the amino acid sequence of SEQ ID NO: 79 and the L chain of the H/L chain pair comprising the amino acid sequence of SEQ ID NO: 78. In some embodiments, the antibody is an anti-TGFβ2 antibody, wherein the antibody comprises: (a) heavy chain CDRs comprising CDR-H1, -H2 and -H3, wherein CDR-H1 has the amino acid sequence of SEQ ID NO: 16, CDR-H2 has the amino acid sequence of SEQ ID NO: 17, and CDR-H3 has the amino acid sequence of SEQ ID NO: 18; and (b) light chain CDRs comprising CDR-L1, -L2 and -L3, wherein CDR-L1 has the amino acid sequence of SEQ ID NO: 19; CDR-L2 has the amino acid sequence of SEQ ID NO: 20, and CDR-L3 has the amino acid sequence of SEQ ID NO: 21. In some embodiments, the antibody is an anti-TGFβ2/3 antibody comprising (i) heavy chain CDRs comprising CDR-H1, -H2 and -H3, wherein CDR-H1 has the amino acid sequence of SEQ ID NO: 10, CDR-H2 has the amino acid sequence of SEQ ID NO: 11, and CDR-H3 has the amino acid sequence of SEQ ID NO: 12; and (ii) light chain CDRs comprising CDR-L1, -L2 and -L3, wherein CDR-L1 has the amino acid sequence of SEQ ID NO: 13; CDR-L2 has the amino acid sequence of SEQ ID NO: 14, and CDR-L3 has the amino acid sequence of SEQ ID NO: 15.

In another aspect, a pharmaceutical formulation or immunoconjugate comprising the antibody of any one of the above aspects and embodiments and a pharmaceutically acceptable carrier is provided. In some embodiments, the antibody is an anti-TGFβ3 antibody comprising: (a1) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 5, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (a2) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 34, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (a3) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 35, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; or (a4) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; or (b) a VH/VL pair, the VH of the VH/VL pair comprising the amino acid sequence of SEQ ID NO: 57, and the VL of the VH/VL pair comprising the amino acid sequence of SEQ ID NO: 56; or (c) a complete H/L chain pair, the H chain of the H/L chain pair comprising the amino acid sequence of SEQ ID NO: 79 and the L chain of the H/L chain pair comprising the amino acid sequence of SEQ ID NO: 78. In some embodiments, the antibody is an anti-TGFβ2 antibody, wherein the antibody comprises: (a) heavy chain CDRs comprising CDR-H1, -H2 and -H3, wherein CDR-H1 has the amino acid sequence of SEQ ID NO: 16, CDR-H2 has the amino acid sequence of SEQ ID NO: 17, and CDR-H3 has the amino acid sequence of SEQ ID NO: 18; and (b) light chain CDRs comprising CDR-L1, -L2 and -L3, wherein CDR-L1 has the amino acid sequence of SEQ ID NO: 19; CDR-L2 has the amino acid sequence of SEQ ID NO: 20, and CDR-L3 has the amino acid sequence of SEQ ID NO: 21.

In some embodiments of the pharmaceutical formulation or conjugate, the antibody is an anti-TGFβ2/3 antibody comprising (i) heavy chain CDRs comprising CDR-H1, -H2 and -H3, wherein CDR-H1 has the amino acid sequence of SEQ ID NO: 10, CDR-H2 has the amino acid sequence of SEQ ID NO: 11, and CDR-H3 has the amino acid sequence of SEQ ID NO: 12; and (ii) light chain CDRs comprising CDR-L1, -L2 and -L3, wherein CDR-L1 has the amino acid sequence of SEQ ID NO: 13; CDR-L2 has the amino acid sequence of SEQ ID NO: 14, and CDR-L3 has the amino acid sequence of SEQ ID NO: 15.

In some aspects, the pharmaceutical formulation further comprises an additional therapeutic agent. In some aspects, the additional therapeutic agent is selected from the group consisting of pirfenidone, nintedanib, mycophenylate mofetil, an IL-6 inhibitor (e.g., tocilizumab), an anti-CTFG antibody (e.g., FG-3019), an autotaxin inhibitor, a JAK inhibitor, an IL-11 inhibitor, and PTX2.

Also provided is an antibody according to any of the above aspects and embodiments for use as a medicament. In some embodiments, the antibody for use as a medicament is an anti-TGFβ3 antibody comprising: (a1) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 5, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (a2) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 34, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (a3) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 35, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; or (a4) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; or (b) light chain CDRs comprising CDR-L1, -L2 and -L3, wherein CDR-L1 has the amino acid sequence of SEQ ID NO: 19; CDR-L2 has the amino acid sequence of SEQ ID NO: 20, and CDR-L3 has the amino acid sequence of SEQ ID NO: 21. In some embodiments, the antibody for use as a medicament is an anti-TGFβ2/3 antibody comprising (i) heavy chain CDRs comprising CDR-H1, -H2 and -H3, wherein CDR-H1 has the amino acid sequence of SEQ ID NO: 10, CDR-H2 has the amino acid sequence of SEQ ID NO: 11, and CDR-H3 has the amino acid sequence of SEQ ID NO: 12; and (ii) light chain CDRs comprising CDR-L1, -L2 and -L3, wherein CDR-L1 has the amino acid sequence of SEQ ID NO: 13; CDR-L2 has the amino acid sequence of SEQ ID NO: 14, and CDR-L3 has the amino acid sequence of SEQ ID NO: 15.

Also provided is an antibody according to any of the above aspects and embodiments for use in treating a TGFβ-related disorder. In some embodiments, the antibody for use in treating a TGFβ-related disorder is an anti-TGFβ3 antibody comprising: (a1) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 5, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (a2) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 34, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (a3) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 35, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; or (a4) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; or (b) a VH/VL pair, the VH of the VH/VL pair comprising the amino acid sequence of SEQ ID NO: 57, and the VL of the VH/VL pair comprising the amino acid sequence of SEQ ID NO: 56; or (c) a complete H/L chain pair, the H chain of the H/L chain pair comprising the amino acid sequence of SEQ ID NO: 79 and the L chain of the H/L chain pair comprising the amino acid sequence of SEQ ID NO: 78. In some embodiments, the antibody for use in treating a TGFβ-related disorder is an anti-TGFβ2 antibody, wherein the antibody comprises: (a) heavy chain CDRs comprising CDR-H1, -H2 and -H3, wherein CDR-H1 has the amino acid sequence of SEQ ID NO: 16, CDR-H2 has the amino acid sequence of SEQ ID NO: 17, and CDR-H3 has the amino acid sequence of SEQ ID NO: 18; and (b) light chain CDRs comprising CDR-L1, -L2 and -L3, wherein CDR-L1 has the amino acid sequence of SEQ ID NO: 19; CDR-L2 has the amino acid sequence of SEQ ID NO: 20, and CDR-L3 has the amino acid sequence of SEQ ID NO: 21. In some embodiments, the antibody for use in treating a TGFβ-related disorder is an anti-TGFβ2/3 antibody comprising (i) heavy chain CDRs comprising CDR-H1, -H2 and -H3, wherein CDR-H1 has the amino acid sequence of SEQ ID NO: 10, CDR-H2 has the amino acid sequence of SEQ ID NO: 11, and CDR-H3 has the amino acid sequence of SEQ ID NO: 12; and (ii) light chain CDRs comprising CDR-L1, -L2 and -L3, wherein CDR-L1 has the amino acid sequence of SEQ ID NO: 13; CDR-L2 has the amino acid sequence of SEQ ID NO: 14, and CDR-L3 has the amino acid sequence of SEQ ID NO: 15.

In one aspect, an anti-TGFβ3 antibody according to any of the above aspects and embodiments is provided and an anti-TGFβ2 antibody according to any of the above aspects and embodiments is provided for use in combination to treat a TGFβ-related disorder. In one embodiment, the anti-TGFβ3 antibody for use in such combination comprises: (a1) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 5, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (a2) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 34, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (a3) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 35, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; or (a4) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; or (b) a VH/VL pair, the VH of the VH/VL pair comprising the amino acid sequence of SEQ ID NO: 57, and the VL of the VH/VL pair comprising the amino acid sequence of SEQ ID NO: 56; or (c) a complete H/L chain pair, the H chain of the H/L chain pair comprising the amino acid sequence of SEQ ID NO: 79 and the L chain of the H/L chain pair comprising the amino acid sequence of SEQ ID NO: 78. In another embodiment, the anti-TGFβ2 antibody comprises: (a) heavy chain CDRs comprising CDR-H1, -H2 and -H3, wherein CDR-H1 has the amino acid sequence of SEQ ID NO: 16, CDR-H2 has the amino acid sequence of SEQ ID NO: 17, and CDR-H3 has the amino acid sequence of SEQ ID NO: 18; and (b) light chain CDRs comprising CDR-L1, -L2 and -L3, wherein CDR-L1 has the amino acid sequence of SEQ ID NO: 19; CDR-L2 has the amino acid sequence of SEQ ID NO: 20, and CDR-L3 has the amino acid sequence of SEQ ID NO: 21. In one aspect, the anti-TGFβ2 antibody selectively neutralizes TGFβ2. In some aspects, the anti-TGFβ2 antibody: (a) has reduced toxicity relative to the pan-TGFβ antibody 1D11; (b) has reduced toxicity in rodents relative to the pan-TGFβ antibody 1D11; (c) has reduced toxicity relative to the pan-TGFβ small molecule inhibitor galunisertib; and/or (d) has reduced toxicity in rodents relative to the pan-TGFβ small molecule inhibitor galunisertib. In one aspect, the anti-TGFβ2 antibody comprises a VH amino acid sequence having at least 95%, at least 96%, at least 97%, at least 98%, at least 99%, or 100% sequence identity to an amino acid sequence selected from the group consisting of SEQ ID NOs: 25, 132, and 138-142. In one aspect, the anti-TGFβ2 antibody comprises a VH amino acid sequence selected from the group consisting of SEQ ID NOs: 25, 132, and 138-142.

Also provided is an antibody according to any of the above aspects and embodiments for use in the manufacture of a medicament for treating a TGFβ-related disorder, for inhibiting TGFBR-dependent SMAD signaling, for inhibiting the assembly of TGFβ-TGFBR signaling complexes, for inhibiting TGFβ signaling through the TGFBR1/R2 complex, for inhibiting TGFβ signaling through the TGFBR2/ALK1 complex facilitated by endoglin, and/or for inhibiting new collagen synthesis. In one embodiment, the antibody for such medicament is an anti-TGFβ3 antibody comprising: (a1) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 5, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (a2) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 34, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (a3) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 35, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; or (a4) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; or (b) a VH/VL pair, the VH of the VH/VL pair comprising the amino acid sequence of SEQ ID NO: 57, and the VL of the VH/VL pair comprising the amino acid sequence of SEQ ID NO: 56; or (c) a complete H/L chain pair, the H chain of the H/L chain pair comprising the amino acid sequence of SEQ ID NO: 79 and the L chain of the H/L chain pair comprising the amino acid sequence of SEQ ID NO: 78. In another embodiment, the antibody for such medicament is an anti-TGFβ2 antibody comprising: (a) heavy chain CDRs comprising CDR-H1, -H2 and -H3, wherein CDR-H1 has the amino acid sequence of SEQ ID NO: 16, CDR-H2 has the amino acid sequence of SEQ ID NO: 17, and CDR-H3 has the amino acid sequence of SEQ ID NO: 18; and (b) light chain CDRs comprising CDR-L1, -L2 and -L3, wherein CDR-L1 has the amino acid sequence of SEQ ID NO: 19; CDR-L2 has the amino acid sequence of SEQ ID NO: 20, and CDR-L3 has the amino acid sequence of SEQ ID NO: 21. In one aspect, the anti-TGFβ2 antibody selectively neutralizes TGFβ2. In some aspects, the anti-TGFβ2 antibody: (a) has reduced toxicity relative to the pan-TGFβ antibody 1D11; (b) has reduced toxicity in rodents relative to the pan-TGFβ antibody 1D11; (c) has reduced toxicity relative to the pan-TGFβ small molecule inhibitor galunisertib; and/or (d) has reduced toxicity in rodents relative to the pan-TGFβ small molecule inhibitor galunisertib. In one aspect, the anti-TGFβ2 antibody comprises a VH amino acid sequence having at least 95%, at least 96%, at least 97%, at least 98%, at least 99%, or 100% sequence identity to an amino acid sequence selected from the group consisting of SEQ ID NOs: 25, 132, and 138-142. In one aspect, the anti-TGFβ2 antibody comprises a VH amino acid sequence selected from the group consisting of SEQ ID NOs: 25, 132, and 138-142. In another aspect, the antibody is an anti-TGFβ2/3 antibody comprising (i) heavy chain CDRs comprising CDR-H1, -H2 and -H3, wherein CDR-H1 has the amino acid sequence of SEQ ID NO: 10, CDR-H2 has the amino acid sequence of SEQ ID NO: 11, and CDR-H3 has the amino acid sequence of SEQ ID NO: 12; and (ii) light chain CDRs comprising CDR-L1, -L2 and -L3, wherein CDR-L1 has the amino acid sequence of SEQ ID NO: 13; CDR-L2 has the amino acid sequence of SEQ ID NO: 14, and CDR-L3 has the amino acid sequence of SEQ ID NO: 15.

In some embodiments of the above uses and medicaments for treating TGFβ-related disorders, the TGFβ-related disorder is fibrosis. In some embodiments, the fibrosis is a fibrotic condition of the lung, liver, heart, kidney, pancreas, eye, and/or skin. In some aspects, the fibrosis is a lung fibrosis selected from the group consisting of idiopathic pulmonary fibrosis (IPF), idiopathic pulmonary upper lobe fibrosis (Amitani disease), familial pulmonary fibrosis, pulmonary fibrosis (e.g., pulmonary fibrosis secondary to systemic inflammatory diseases such as, rheumatoid arthritis, scleroderma, lupus, cryptogenic fibrosing alveolitis, chronic obstructive pulmonary disease (COPD) or chronic asthma), cystic fibrosis, non-specific interstitial pneumonia (NSIP), cryptogenic organizing pneumonia (COP), progressive massive fibrosis, scleroderma/systemic sclerosis (SSc, including limited cutaneous (IcSSc) and diffuse cutaneous (dcSSc) forms and SSc-associated interstitial lung disease (SSc-ILD)), bronchiolitis obliterans-organizing pneumonia, connective tissue disease-associated ILD (CT-ILD), hypersensitivity pneumonitis, pulmonary hypertension, pulmonary tuberculosis, silicosis, asbestosis, acute lung injury, and acute respiratory distress (ARD, including bacterial pneumonia induced, trauma-induced, and viral pneumonia-induced, ventilator-induced, non-pulmonary sepsis induced). In some aspects, the fibrosis is a fibrotic condition of the liver selected from the group consisting of liver cirrhosis, congenital hepatic fibrosis, obesity, fatty liver, alcohol induced liver fibrosis, non-alcoholic steatohepatitis (NASH), biliary duct injury, primary biliary cirrhosis (PBC), primary sclerosing cholangitis (PSC), infection- or viral-induced liver fibrosis (e.g., chronic hepatitis B and C virus infections), cystic fibrosis, autoimmune hepatitis, necrotizing hepatitis, primary sclerosing cholangitis, hemochromatosis, disorders of the biliary tree, and hepatic dysfunction attributable to infections. In some aspects, the fibrosis is a fibrotic condition of the heart and/or pericardium selected from the group consisting of endomyocardial fibrosis, cardiac allograft vasculopathy (CAV), myocardial infarction, atrial fibrosis, congestive heart failure, arteriosclerosis, atherosclerosis, vascular stenosis, myocarditis, congestive cardiomyopathy, coronary infarcts, varicose veins, coronary artery stenosis and other post-ischemic conditions, and idiopathic retroperitoneal fibrosis. In some aspects, the fibrosis is a fibrotic condition of the kidney selected from the group consisting of glomerulonephritis (including membranoproliferative, diffuse proliferative, rapidly progressive or sclerosing, post-infectious and chronic forms), diabetic glomerulosclerosis, focal segmental glomerulosclerosis, IgA nephropathy, diabetic nephropathy, ischemic nephropathy, tubulointerstitial kidney fibrosis, HIV-associated nephropathy, membrane nephropathy, glomerulonephritis secondary to systemic inflammatory diseases such as lupus, scleroderma and diabetes glomerulonephritis, idiopathic membranoproliferative glomerular nephritis, mesangial proliferative glomerulonephritis, crescentic glomerulonephritis, amyloidosis (which affects the kidney among other tissues), autoimmune nephritis, renal tubulointerstitial fibrosis, renal arteriosclerosis, Alport's syndrome, nephrotic syndrome, chronic renal failure, chronic kidney disease, periglomerular fibrosis/atubular glomeruli, combined apical emphysema and basal fibrosis syndrome (emphysema/fibrosis syndrome), glomerular hypertension, nephrogenic fibrosing dermatopathy, polycystic kidney disease, Fabry's disease, and renal hypertension. In some aspects, the fibrosis is a fibrotic condition of the pancreas selected from the group consisting of stromal remodeling pancreatitis and stromal fibrosis. In some aspects, the fibrosis is a fibrotic condition of the gastrointestinal tract selected from the group consisting of Crohn's disease, ulcerative colitis, collagenous colitis, colorectal fibrosis, villous atrophy, crypt hyperplasia, polyp formation, healing gastric ulcer, and microscopic colitis. In some aspects, the fibrosis is a fibrotic condition of the eye selected from the group consisting of ocular fibrosis, ophthalmic fibrosis, proliferative vitreoretinopathy, vitreoretinopathy of any etiology, fibrosis associated with retinal dysfunction, fibrosis associated with wet or dry macular degeneration, scarring in the cornea and conjunctiva, fibrosis in the corneal endothelium, anterior subcapsular cataract and posterior capsule opacification, anterior segment fibrotic diseases of the eye, fibrosis of the corneal stroma (e.g., associated with corneal opacification), fibrosis of the trabecular network (e.g., associated with glaucoma), posterior segment fibrotic diseases of the eye, fibrovascular scarring (e.g., in retinal or choroidal vasculature of the eye), retinal fibrosis, epiretinal fibrosis, retinal gliosis, subretinal fibrosis (e.g., associated with age related macular degeneration), tractional retinal detachment in association with contraction of the tissue in diabetic retinopathy, congenital orbital fibrosis, lacrimal gland fibrosis, corneal subepithelial fibrosis, and Grave's ophthalmopathy. In some aspects, the fibrosis is selected from fibrosis resulting from spinal cord injury/fibrosis or central nervous system fibrosis such as fibrosis after a stroke, Duchenne muscular dystrophy, fibrosis associated with neurodegenerative disorder such as Alzheimer's disease or multiple sclerosis, vascular restenosis, uterine fibrosis, endometriosis, ovarian fibroids, Peyronie's disease, polycystic ovarian syndrome, disease related pulmonary apical fibrosis in ankylosing spondylitis, scarring, and fibrosis incident to microbial infections (e.g., bacterial, viral, parasitic, fungal). In a specific embodiment, the fibrosis is SSc. In a specific embodiment, the fibrosis is IPF. In a specific embodiment, the fibrosis is chronic obstructive pulmonary disease (COPD). In a specific embodiment, the fibrosis is progressive-fibrosing interstitial lung disease (PF-ILD). In a specific embodiment, the PF-ILD is a disease or condition selected from the group consisting of non-specific interstitial pneumonia (NSIP), cryptogenic organizing pneumonia (COP), progressive massive fibrosis, a complication of coal worker's pneumoconiosis, scleroderma/systemic sclerosis, bronchiolitis obliterans-organizing pneumonia; connective tissue disease-associated ILD (CT-ILD), and hypersensitivity pneumonitis. In a specific embodiment, the fibrosis is liver cirrhosis or chronic hepatic fibrosis. In a specific embodiment, the fibrosis is GI tract fibrosis. In a specific embodiment, the fibrosis is a fibrotic condition of the eye, fibrosis resulting from spinal cord injury, fibrosis or central nervous system fibrosis, or fibrosis associated with a neurodegenerative disorder.

In another aspect, a method of treating a subject having a TGFβ-related disorder is provided. In some embodiments, the method comprises administering an effective amount of an antibody or pharmaceutical formulation according to any of the above aspects and embodiments to a subject in need thereof. In another aspect a method for inhibiting TGFBR-dependent SMAD signaling, inhibiting the assembly of TGFβ-TGFBR signaling complexes, inhibiting TGFβ signaling through the TGFBR1/R2 complex, inhibiting TGFβ signaling through the TGFBR2/ALK1 complex facilitated by endoglin, and/or for inhibiting new collagen synthesis in a subject is provided. In some aspects, the method includes administering to a subject in need thereof an effective amount of the antibody according to any of the above aspects and embodiments to inhibit TGFBR-dependent SMAD signaling, to inhibit the assembly of TGFβ-TGFBR signaling complexes, to inhibit TGFβ signaling through the TGFBR1/R2 complex, to inhibit TGFβ signaling through the TGFBR2/ALK1 complex facilitated by endoglin and/or to inhibit new collagen synthesis in the subject. In some aspects, the method includes administering an additional therapeutic agent to the subject. In some aspects, the additional therapeutic agent is selected from the group consisting of pirfenidone, nintedanib, mycophenylate mofetil, an IL-6 inhibitor (e.g., tocilizumab, sarilumab), an anti-CTFG antibody (e.g., FG-3019), an autotaxin inhibitor, and PTX2. In one embodiment of such methods, the antibody is an anti-TGFβ3 antibody comprising: (a1) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 5, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (a2) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 34, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (a3) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 35, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; or (a4) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; or (b) a VH/VL pair, the VH of the VH/VL pair comprising the amino acid sequence of SEQ ID NO: 57, and the VL of the VH/VL pair comprising the amino acid sequence of SEQ ID NO: 56; or (c) a complete H/L chain pair, the H chain of the H/L chain pair comprising the amino acid sequence of SEQ ID NO: 79 and the L chain of the H/L chain pair comprising the amino acid sequence of SEQ ID NO: 78. In another embodiment of such methods, the antibody is an anti-TGFβ2 antibody comprising: (a) heavy chain CDRs comprising CDR-H1, -H2 and -H3, wherein CDR-H1 has the amino acid sequence of SEQ ID NO: 16, CDR-H2 has the amino acid sequence of SEQ ID NO: 17, and CDR-H3 has the amino acid sequence of SEQ ID NO: 18; and (b) light chain CDRs comprising CDR-L1, -L2 and -L3, wherein CDR-L1 has the amino acid sequence of SEQ ID NO: 19; CDR-L2 has the amino acid sequence of SEQ ID NO: 20, and CDR-L3 has the amino acid sequence of SEQ ID NO: 21. In one aspect, the anti-TGFβ2 antibody selectively neutralizes TGFβ2. In some aspects, the anti-TGFβ2 antibody: (a) has reduced toxicity relative to the pan-TGFβ antibody 1D11; (b) has reduced toxicity in rodents relative to the pan-TGFβ antibody 1D11; (c) has reduced toxicity relative to the pan-TGFβ small molecule inhibitor galunisertib; and/or (d) has reduced toxicity in rodents relative to the pan-TGFβ small molecule inhibitor galunisertib. In one aspect, the anti-TGFβ2 antibody comprises a VH amino acid sequence having at least 95%, at least 96%, at least 97%, at least 98%, at least 99%, or 100% sequence identity to an amino acid sequence selected from the group consisting of SEQ ID NOs: 25, 132, and 138-142. In one aspect, the anti-TGFβ2 antibody comprises a VH amino acid sequence selected from the group consisting of SEQ ID NOs: 25, 132, and 138-142. In another embodiment of such methods, the antibody is an anti-TGFβ2/3 antibody comprising (i) heavy chain CDRs comprising CDR-H1, -H2 and -H3, wherein CDR-H1 has the amino acid sequence of SEQ ID NO: 10, CDR-H2 has the amino acid sequence of SEQ ID NO: 11, and CDR-H3 has the amino acid sequence of SEQ ID NO: 12; and (ii) light chain CDRs comprising CDR-L1, -L2 and -L3, wherein CDR-L1 has the amino acid sequence of SEQ ID NO: 13; CDR-L2 has the amino acid sequence of SEQ ID NO: 14, and CDR-L3 has the amino acid sequence of SEQ ID NO: 15.

In a further aspect of the above methods, the method can include administering to the subject an effective amount of an anti-TGFβ3 antibody and an effective amount of an anti-TGFβ2 antibody. In one embodiment, the anti-TGFβ3 antibody for use in such combination comprises: (a1) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 5, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (a2) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 34, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (a3) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 35, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; or (a4) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; or (b) a VH/VL pair, the VH of the VH/VL pair comprising the amino acid sequence of SEQ ID NO: 57, and the VL of the VH/VL pair comprising the amino acid sequence of SEQ ID NO: 56; or (c) a complete H/L chain pair, the H chain of the H/L chain pair comprising the amino acid sequence of SEQ ID NO: 79 and the L chain of the H/L chain pair comprising the amino acid sequence of SEQ ID NO: 78. In another embodiment, the anti-TGFβ2 antibody comprises: (a) heavy chain CDRs comprising CDR-H1, -H2 and -H3, wherein CDR-H1 has the amino acid sequence of SEQ ID NO: 16, CDR-H2 has the amino acid sequence of SEQ ID NO: 17, and CDR-H3 has the amino acid sequence of SEQ ID NO: 18; and (b) light chain CDRs comprising CDR-L1, -L2 and -L3, wherein CDR-L1 has the amino acid sequence of SEQ ID NO: 19; CDR-L2 has the amino acid sequence of SEQ ID NO: 20, and CDR-L3 has the amino acid sequence of SEQ ID NO: 21. In one aspect, the anti-TGFβ2 antibody selectively neutralizes TGFβ2. In some aspects, the anti-TGFβ2 antibody: (a) has reduced toxicity relative to the pan-TGFβ antibody 1D11; (b) has reduced toxicity in rodents relative to the pan-TGFβ antibody 1D11; (c) has reduced toxicity relative to the pan-TGFβ small molecule inhibitor galunisertib; and/or (d) has reduced toxicity in rodents relative to the pan-TGFβ small molecule inhibitor galunisertib. In one aspect, the anti-TGFβ2 antibody comprises a VH amino acid sequence having at least 95%, at least 96%, at least 97%, at least 98%, at least 99%, or 100% sequence identity to an amino acid sequence selected from the group consisting of SEQ ID NOs: 25, 132, and 138-142. In one aspect, the anti-TGFβ2 antibody comprises a VH amino acid sequence selected from the group consisting of SEQ ID NOs: 25, 132, and 138-142.

In further embodiments of the above methods for treating TGFβ-related disorders, the TGFβ-related disorder can be fibrosis. In some embodiments, the fibrosis is a fibrotic condition of the lung, liver, heart, kidney, pancreas, eye, and/or skin. In some aspects, the fibrosis is a lung fibrosis selected from the group consisting of idiopathic pulmonary fibrosis (IPF), idiopathic pulmonary upper lobe fibrosis (Amitani disease), familial pulmonary fibrosis, pulmonary fibrosis (e.g., pulmonary fibrosis secondary to systemic inflammatory diseases such as, rheumatoid arthritis, scleroderma, lupus, cryptogenic fibrosing alveolitis, chronic obstructive pulmonary disease (COPD) or chronic asthma), cystic fibrosis, non-specific interstitial pneumonia (NSIP), cryptogenic organizing pneumonia (COP), progressive massive fibrosis, scleroderma/systemic sclerosis (SSc, including limited cutaneous (IcSSc) and diffuse cutaneous (dcSSc) forms and SSc-associated interstitial lung disease (SSc-ILD)), bronchiolitis obliterans-organizing pneumonia, connective tissue disease-associated ILD (CT-ILD), hypersensitivity pneumonitis, pulmonary hypertension, pulmonary tuberculosis, silicosis, asbestosis, acute lung injury, and acute respiratory distress (ARD, including bacterial pneumonia induced, trauma-induced, and viral pneumonia-induced, ventilator-induced, non-pulmonary sepsis induced). In some aspects, the fibrosis is a fibrotic condition of the liver selected from the group consisting of liver cirrhosis, congenital hepatic fibrosis, obesity, fatty liver, alcohol induced liver fibrosis, non-alcoholic steatohepatitis (NASH), biliary duct injury, primary biliary cirrhosis (PBC), primary sclerosing cholangitis (PSC), infection- or viral-induced liver fibrosis (e.g., chronic hepatitis B and C virus infections), cystic fibrosis, autoimmune hepatitis, necrotizing hepatitis, primary sclerosing cholangitis, hemochromatosis, disorders of the biliary tree, and hepatic dysfunction attributable to infections. In some aspects, the fibrosis is a fibrotic condition of the heart and/or pericardium selected from the group consisting of endomyocardial fibrosis, cardiac allograft vasculopathy (CAV), myocardial infarction, atrial fibrosis, congestive heart failure, arteriosclerosis, atherosclerosis, vascular stenosis, myocarditis, congestive cardiomyopathy, coronary infarcts, varicose veins, coronary artery stenosis and other post-ischemic conditions, and idiopathic retroperitoneal fibrosis. In some aspects, the fibrosis is a fibrotic condition of the kidney selected from the group consisting of glomerulonephritis (including membranoproliferative, diffuse proliferative, rapidly progressive or sclerosing, post-infectious and chronic forms), diabetic glomerulosclerosis, focal segmental glomerulosclerosis, IgA nephropathy, diabetic nephropathy, ischemic nephropathy, tubulointerstitial kidney fibrosis, HIV-associated nephropathy, membrane nephropathy, glomerulonephritis secondary to systemic inflammatory diseases such as lupus, scleroderma and diabetes glomerulonephritis, idiopathic membranoproliferative glomerular nephritis, mesangial proliferative glomerulonephritis, crescentic glomerulonephritis, amyloidosis (which affects the kidney among other tissues), autoimmune nephritis, renal tubulointerstitial fibrosis, renal arteriosclerosis, Alport's syndrome, nephrotic syndrome, chronic renal failure, chronic kidney disease, periglomerular fibrosis/atubular glomeruli, combined apical emphysema and basal fibrosis syndrome (emphysema/fibrosis syndrome), glomerular hypertension, nephrogenic fibrosing dermatopathy, polycystic kidney disease, Fabry's disease, and renal hypertension. In some aspects, the fibrosis is a fibrotic condition of the pancreas selected from the group consisting of stromal remodeling pancreatitis and stromal fibrosis. In some aspects, the fibrosis is a fibrotic condition of the gastrointestinal tract selected from the group consisting of Crohn's disease, ulcerative colitis, collagenous colitis, colorectal fibrosis, villous atrophy, crypt hyperplasia, polyp formation, healing gastric ulcer, and microscopic colitis. In some aspects, the fibrosis is a fibrotic condition of the eye selected from the group consisting of ocular fibrosis, ophthalmic fibrosis, proliferative vitreoretinopathy, vitreoretinopathy of any etiology, fibrosis associated with retinal dysfunction, fibrosis associated with wet or dry macular degeneration, scarring in the cornea and conjunctiva, fibrosis in the corneal endothelium, anterior subcapsular cataract and posterior capsule opacification, anterior segment fibrotic diseases of the eye, fibrosis of the corneal stroma (e.g., associated with corneal opacification), fibrosis of the trabecular network (e.g., associated with glaucoma), posterior segment fibrotic diseases of the eye, fibrovascular scarring (e.g., in retinal or choroidal vasculature of the eye), retinal fibrosis, epiretinal fibrosis, retinal gliosis, subretinal fibrosis (e.g., associated with age related macular degeneration), tractional retinal detachment in association with contraction of the tissue in diabetic retinopathy, congenital orbital fibrosis, lacrimal gland fibrosis, corneal subepithelial fibrosis, and Grave's ophthalmopathy. In some aspects, the fibrosis is selected from fibrosis resulting from spinal cord injury/fibrosis or central nervous system fibrosis such as fibrosis after a stroke, Duchenne muscular dystrophy, fibrosis associated with neurodegenerative disorder such as Alzheimer's disease or multiple sclerosis, vascular restenosis, uterine fibrosis, endometriosis, ovarian fibroids, Peyronie's disease, polycystic ovarian syndrome, disease related pulmonary apical fibrosis in ankylosing spondylitis, scarring, and fibrosis incident to microbial infections (e.g., bacterial, viral, parasitic, fungal). In a specific embodiment, the fibrosis is SSc. In a specific embodiment, the fibrosis is IPF. In a specific embodiment, the fibrosis is chronic obstructive pulmonary disease (COPD). In a specific embodiment, the fibrosis is progressive-fibrosing interstitial lung disease (PF-ILD). In a specific embodiment, the PF-ILD is a disease or condition selected from the group consisting of non-specific interstitial pneumonia (NSIP), cryptogenic organizing pneumonia (COP), progressive massive fibrosis, a complication of coal worker's pneumoconiosis, scleroderma/systemic sclerosis, bronchiolitis obliterans-organizing pneumonia; connective tissue disease-associated ILD (CT-ILD), and hypersensitivity pneumonitis. In a specific embodiment, the fibrosis is liver cirrhosis or chronic hepatic fibrosis. In a specific embodiment, the fibrosis is GI tract fibrosis, e.g., intestinal fibrosis, optionally, selected from the group consisting of fibrosis associated with Crohn's disease, ulcerative colitis, collagenous colitis, colorectal fibrosis, villous atrophy, crypt hyperplasia, polyp formation, healing gastric ulcer, and microscopic colitis. In a specific embodiment, the fibrosis is a fibrotic condition of the eye, fibrosis resulting from spinal cord injury, fibrosis or central nervous system fibrosis, or fibrosis associated with a neurodegenerative disorder.

In another aspect, a method of diagnosing a subject as having SSc is provided. In some embodiments, the method includes detecting the expression levels of the genes in an 18-gene signature set consisting of PRSS23, PXDN, COL8A1, COL6A3, SERPINE2, TNC, COMP, THBS1, COL11A1, COL1A1, COL5A2, COL1A2, COL4A1, COL4A2, SFRP4, ALPK2, COL5A1, and TAGLN, and diagnosing the subject with SSc if the levels of the genes are determined to be elevated relative to the gene levels in a healthy control set or reference gene signature. In some aspects, a gene level is elevated if the expression increase relative to the healthy control set or reference gene signature is statistically significant, optionally, at least two-fold increased, or at least three-fold increased, or at least four-fold increased, relative to the healthy control set or reference gene signature. In some aspects, the gene expression level is detected using qPCR or microarray or RNAseq.

In another aspect, a method of monitoring response to treatment of a subject with an anti-TGFβ2 antibody and/or an anti-TGFβ3 antibody is provided. In some aspects, the method includes determining the expression level of one or more of the TGFβ-inducible genes selected from the group consisting of serpine1, col1a1, col1a2, and col3a1 in a sample from the subject, wherein the subject has received one or more administrations of the anti-TGFβ2 antibody and/or the anti-TGFβ3 antibody. In some aspects, the subject is determined to be responding to treatment with the anti-TGFβ2 antibody and/or anti-TGFβ3 antibody, if the expression level of the one or more TGFβ-inducible genes is significantly reduced compared to pre-treatment levels of the one or more TGFβ-inducible genes, wherein optionally the method further comprises administering one or more additional administrations of the anti-TGFβ2 antibody and/or the anti-TGFβ3 antibody if the expression level of the one or more TGFβ-inducible genes is determined to be significantly reduced. In some aspects, the subject is administered the anti-TGFβ2 antibody as a monotherapy. In some aspects, the subject is administered the anti-TGFβ3 antibody as a monotherapy. In some aspects, the expression level of the one or more TGFβ-inducible genes is determined by qPCR or microarray analysis. In one embodiment of the method, the antibody is an anti-TGFβ3 antibody comprising: (a1) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 5, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (a2) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 34, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (a3) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 35, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; or (a4) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; or (b) a VH/VL pair, the VH of the VH/VL pair comprising the amino acid sequence of SEQ ID NO: 57, and the VL of the VH/VL pair comprising the amino acid sequence of SEQ ID NO: 56; or (c) a complete H/L chain pair, the H chain of the H/L chain pair comprising the amino acid sequence of SEQ ID NO: 79 and the L chain of the H/L chain pair comprising the amino acid sequence of SEQ ID NO: 78. In another embodiment of the method, the antibody is an anti-TGFβ2 antibody comprising: (a) heavy chain CDRs comprising CDR-H1, -H2 and -H3, wherein CDR-H1 has the amino acid sequence of SEQ ID NO: 16, CDR-H2 has the amino acid sequence of SEQ ID NO: 17, and CDR-H3 has the amino acid sequence of SEQ ID NO: 18; and (b) light chain CDRs comprising CDR-L1, -L2 and -L3, wherein CDR-L1 has the amino acid sequence of SEQ ID NO: 19; CDR-L2 has the amino acid sequence of SEQ ID NO: 20, and CDR-L3 has the amino acid sequence of SEQ ID NO: 21. In one aspect, the anti-TGFβ2 antibody selectively neutralizes TGFβ2. In some aspects, the anti-TGFβ2 antibody: (a) has reduced toxicity relative to the pan-TGFβ antibody 1D11; (b) has reduced toxicity in rodents relative to the pan-TGFβ antibody 1D11; (c) has reduced toxicity relative to the pan-TGFβ small molecule inhibitor galunisertib; and/or (d) has reduced toxicity in rodents relative to the pan-TGFβ small molecule inhibitor galunisertib. In one aspect, the anti-TGFβ2 antibody comprises a VH amino acid sequence having at least 95%, at least 96%, at least 97%, at least 98%, at least 99%, or 100% sequence identity to an amino acid sequence selected from the group consisting of SEQ ID NOs: 25, 132, and 138-142. In one aspect, the anti-TGFβ2 antibody comprises a VH amino acid sequence selected from the group consisting of SEQ ID NOs: 25, 132, and 138-142.

In any of the above aspects and embodiments for uses, medicaments, and methods of treatment and diagnosis and monitoring response to treatment, the subject can be a human, e.g., a human patient.

In another aspect, a kit comprising an antibody of any of the above aspects and embodiments is provided. In one embodiment of the kit, the antibody is an anti-TGFβ3 antibody comprising: (a1) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 5, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (a2) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 34, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (a3) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 35, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; or (a4) (i) heavy chain CDRs comprising CDR-H1, -H2 and -H3, wherein CDR-H1 has the amino acid sequence of SEQ ID NO: 4, CDR-H2 has the amino acid sequence of SEQ ID NO: 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; or (b) a VH/VL pair, the VH of the VH/VL pair comprising the amino acid sequence of SEQ ID NO: 57, and the VL of the VH/VL pair comprising the amino acid sequence of SEQ ID NO: 56; or (c) a complete H/L chain pair, the H chain of the H/L chain pair comprising the amino acid sequence of SEQ ID NO: 79 and the L chain of the H/L chain pair comprising the amino acid sequence of SEQ ID NO: 78. In another embodiment of the kit, the antibody is an anti-TGFβ2 antibody comprising: (a) heavy chain CDRs comprising CDR-H1, -H2 and -H3, wherein CDR-H1 has the amino acid sequence of SEQ ID NO: 16, CDR-H2 has the amino acid sequence of SEQ ID NO: 17, and CDR-H3 has the amino acid sequence of SEQ ID NO: 18; and (b) light chain CDRs comprising CDR-L1, -L2 and -L3, wherein CDR-L1 has the amino acid sequence of SEQ ID NO: 19; CDR-L2 has the amino acid sequence of SEQ ID NO: 20, and CDR-L3 has the amino acid sequence of SEQ ID NO: 21. In one aspect, the anti-TGFβ2 antibody selectively neutralizes TGFβ2. In some aspects, the anti-TGFβ2 antibody: (a) has reduced toxicity relative to the pan-TGFβ antibody 1D11; (b) has reduced toxicity in rodents relative to the pan-TGFβ antibody 1D11; (c) has reduced toxicity relative to the pan-TGFβ small molecule inhibitor galunisertib; and/or (d) has reduced toxicity in rodents relative to the pan-TGFβ small molecule inhibitor galunisertib. In one aspect, the anti-TGFβ2 antibody comprises a VH amino acid sequence having at least 95%, at least 96%, at least 97%, at least 98%, at least 99%, or 100% sequence identity to an amino acid sequence selected from the group consisting of SEQ ID NOs: 25, 132, and 138-142. In one aspect, the anti-TGFβ2 antibody comprises a VH amino acid sequence selected from the group consisting of SEQ ID NOs: 25, 132, and 138-142. In another embodiment of the kit, the antibody is an anti-TGFβ2/3 antibody comprising (i) heavy chain CDRs comprising CDR-H1, -H2 and -H3, wherein CDR-H1 has the amino acid sequence of SEQ ID NO: 10, CDR-H2 has the amino acid sequence of SEQ ID NO: 11, and CDR-H3 has the amino acid sequence of SEQ ID NO: 12; and (ii) light chain CDRs comprising CDR-L1, -L2 and -L3, wherein CDR-L1 has the amino acid sequence of SEQ ID NO: 13; CDR-L2 has the amino acid sequence of SEQ ID NO: 14, and CDR-L3 has the amino acid sequence of SEQ ID NO: 15.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows the primary amino acid sequence alignment of human TGFβ1, 2, and 3 having accession numbers as follows: huTGFβ1: XP_011525544.1, huTGFβ2: NP_003229.1, huTGFβ3: ABQ59024.1, with domains of TGFβ1 (α-sheets and β-strands, latency lasso, fastener, integrin-binding, furin cleavage, and cytokine) indicated as per Shi et al., Nature 474:343 (2011). Residues are numbered from the predicted ATG (methionine) start site; Shi et al. numbering starts from L30 at the start of the α1 helix. The black arrow underneath the sequences indicates the start of the receptor-binding domain. Frameshift, premature stop, and splice site mutations reported for TGFβ2 and TGFβ3 are omitted for clarity. The sequences shown in the figure correspond to SEQ ID NOs: 1-3, respectively.

FIG. 2A shows the pharmacokinetic profile of 6F12 muIgG2a antibody given as a single 1 or 10 mg/kg I.V. and 10 mg/kg I.P. dose in C57BL6 mice (n=3 per timepoint).

FIG. 2B shows the pharmacokinetic profile of 16C10 muIgG2a, 15A7 muIgG2a, 18B5 muIgG2a, 2A10 muIgG2a and non-binding anti-gD huIgG1 control antibody given as a single 10 mg/kg I.V. dose in C57BL6 mice (n=3 per timepoint).

FIG. 2C shows the pharmacokinetic profile of 4A11 muIgG2a given as a single 1 or 10 mg/kg I.V. and 10 mg/kg I.P. dose, and 6F12 muIgG2a antibody given as a single 10 mg/kg I.V. dose in C57BL6 mice (n=3 per timepoint).

FIG. 3A is a graph showing expression levels determined from microarray analysis of TGFβ isoforms in bulk lung biopsy tissue from control (n=8) and IPF (n=40) lungs. The data show elevated expression of TGFβ2 and TGFβ3 but not TGFβ1 in IPF. **P<0.01; **** P <0.0001 (unpaired two-tailed Student's t-tests).

FIG. 3B is an image of pSMAD2/3 immunohistochemistry (IHC) of control and IPF lung tissue showing increased nuclear pSMAD staining in fibroblastic foci of IPF lung tissue.

FIG. 4A is a t-SNE plot representing single cell RNAseq of IPF lungs (n=3, combined), showing representation of multiple epithelial, mesenchymal, and hematopoietic cell lineages as labeled. Expression of individual TGFβ isoforms showed broad expression of TGFβ1, predominantly in hematopoietic and endothelial cells; TGFβ2 expression predominantly in epithelial cells; and TGFβ3 predominantly in mesenchymal cells. Expression of TGFβ target genes showed strong overlap of SERPINE1, COL1A1, POSTN, and COMP with TGFβ3 expression in fibroblasts and myofibroblasts. SERPINE1 also overlaps with TGFβ1 expression in endothelial cells and macrophages, and POSTN in endothelial cells.

FIG. 4B is an image of dual IHC for pSMAD3 and in situ hybridization (ISH) for TGFβ3 showing colocalization of TGFβ3 mRNA and nuclear pSMAD3 in multiple cells in a fibroblastic focus in IPF lung tissue.

FIG. 5A contains plots showing expression of TGFβ isoforms as determined from microarray analysis of skin biopsies taken from healthy controls (HC) and patients with systemic sclerosis (SSc) at baseline enrolled in the FaSScinate trial.

FIG. 5B shows the TGFβR signal dependent skin gene expression signature derived by comparing genes induced in fibroblasts treated in vitro with recombinant TGFβ1 receptor-binding domain and genes significantly upregulated in SSc vs. control skin biopsy. Genes that were significantly elevated in both conditions were selected as a candidate TGFβ signature in SSc skin.

FIG. 6A is a plot showing principal component 1 of the signature defined in FIG. 5B, derived as a continuous variable for magnitude of TGFβR-dependent gene expression across all signature genes in the SSc skin biopsies and compared to TGFβ isoform expression levels in those biopsies. Both “TGFβ-high” and “TGFβ-low” SSc patients had higher levels of this gene signature than healthy controls, and the skin biopsy TGFβ gene signature was highly correlated with TGFβ3, but not TGFβ1 or TGFβ2 expression.

FIG. 6B is a table showing intercorrelations between TGFβ-inducible skin gene signature metric as determined in FIG. 4C and skin gene expression levels of TGFβ isoforms, POSTN, and COMP; and intercorrelations with serum levels of POSTN and COMP proteins in the FaSScinate study.

FIG. 6C is a table summarizing correlation of serum periostin and COMP levels with the Modified Rodnan Skin Score (MRSS), a clinical index of systemic skin fibrosis used in the FaSScinate study.

FIG. 7 contains plots showing distribution of change in MRSS from baseline to 48 weeks of patients in the FaSScinate study stratified by baseline TGFβ skin gene signature cluster. PBO: placebo; TCZ: tocilizumab treatment; Cum Prob: cumulative probability of MRSS change.

FIGS. 8A-F show expression derived from microarray at the indicated times after i.t. bleomycin installation of TGFβ1 (FIG. 8A), TGFβ2 (FIG. 8B), TGFβ3 (FIG. 8C), COL1A1 (FIG. 8D), Serpine1 (FIG. 8E), and Fn1 (FIG. 8F). Expression of TGFβ2, TGFβ3, COL1A1, Serpine1 and Fn1 followed similar kinetics, peaking between day 7-14 after bleomycin. *, P<0.05; **, P<0.01; ***, P<10⁻³; ****, P<10⁴ by unpaired two-tailed Student's t-test. Data represent means±SEM.

FIG. 9A is a schematic for in vivo I.T. bleomycin experiments to assess efficacy of TGFβ antibodies. Separate cohorts of animals were sacrificed at day 14 or day 24 to assess lung gene expression during peak TGFβ2/3 expression or lung collagen toward the end of the ‘fibrotic’ stage, respectively. Deuterated drinking water was provided to animals from day 9-23 to assess the fraction of new collagen production during that interval (deuterated hydroxyproline).

FIG. 9B is a plot showing reduced levels of the fraction of lung deuterated to total hydroxyproline at day 24 with preventive administration of anti-TGFβ2 and/or TGFβ3 antibodies (10 mpk, TIW). Each dot represents an individual animal.

FIG. 9C is a plot showing reduced levels of lung gene expression of FN1 at day 14 after i.t. bleomycin instillation with preventive administration of anti-TGFβ2 and/or TGFβ3 antibodies (10 mpk, TIW). **P<0.01; *** P<0.001; ****P<0.0001 (One-way ANOVA with Dunnett's test).

FIG. 9D and FIG. 9E are plots showing the effect of co-administration of 6F12 and 2A10 (10 mpk, TIW) or 4A11 at 10 mpk TIW, 10 mpk QW, or 2.5 mpk QW on total (FIG. 9D) and deuterated (FIG. 9E) hydroxyproline at day 24 after i.t. bleomycin. Similar reductions observed for 10 mpk of 4A11 QW vs. TIW, but decreased effect at 2.5 mpk QW. **P<0.01; *** P<0.001; ****P<0.0001 (One-way ANOVA with Dunnett's test).

FIG. 10 is a schematic showing the attempts to express various humanized variants of 2A10 mAb.

FIG. 11 is a table showing loss of expression and stability for most of the h2A10v1 variants during framework and CDR polishing.

FIG. 12 contains amino acid sequence alignments of the light chain (upper panel) and heavy chain (lower panel) variable region sequences of the rat 2A10 antibody and its humanized variants v1-v4; the amino acid sequences shown in the figure are SEQ ID NOs: 22, 36, 36, 54, and 56 (upper panel, from top sequence to bottom sequence in the alignment) and SEQ ID NOs: 23, 37, 45, 55, and 57 (lower panel, from top sequence to bottom sequence in the alignment). Residue numbering is according to Kabat; “*” indicates Vernier zone. CDR regions according to Chothia or Kabat (as indicated) are boxed; mutated residues are highlighted.

FIG. 13 contains an amino acid sequence alignment of the light chain variable region of the rat 2A10 and humanized h2A10 v2 variants, v2-v2.9; the sequences shown correspond to SEQ ID NOs: 22, 36, 38, 39, 40, 41, 36, 36, 36, 36, and 36 (from top sequence to bottom sequence in the alignment). Residue numbering is according to Kabat; “*” indicates Vernier zone. CDR regions according to Chothia or Kabat (as indicated) are boxed; mutated residues are highlighted.

FIG. 14 contains an amino acid sequence alignment of the heavy chain variable region of the rat 2A10 and humanized h2A10 v2 variants, v2-v2.9; the sequences shown correspond to SEQ ID NOs: 23, 45, 45, 45, 45, 45, and 46-50 (from top sequence to bottom sequence in the alignment). Residue numbering is according to Kabat; “*” indicates Vernier zone. CDR regions according to Chothia or Kabat (as indicated) are boxed; mutated residues are highlighted.

FIG. 15 contains amino acid sequence alignments of the light chain (upper panel) and heavy chain (lower panel) variable region sequences of the rat 2A10 antibody and humanized v2 variants; the amino acid sequences shown in the figure are SEQ ID NOs: 22, 36, 36, 36, and 36 (upper panel, from top sequence to bottom sequence in the alignment) and SEQ ID NOs: 23, 45, and 51-53 (lower panel, from top sequence to bottom sequence in the alignment). Residue numbering is according to Kabat; “*” indicates Vernier zone. CDR regions according to Chothia or Kabat (as indicated) are boxed; mutated residues are highlighted.

FIG. 16 contains amino acid sequence alignments of the light chain (upper panel) and heavy chain (lower panel) variable region sequences of the rat 2A10 antibody its humanized v4 variant; the amino acid sequences shown in the figure are SEQ ID NOs: 22 and 56 (upper panel, from top sequence to bottom sequence in the alignment) and SEQ ID NOs: 23 and 57 (lower panel, from top sequence to bottom sequence in the alignment). Residue numbering is according to Kabat; “*” indicates Vernier zone. CDR regions according to Chothia or Kabat (as indicated) are boxed; mutated residues are highlighted.

FIG. 17 is a line graph showing mean (±SD) serum concentration of h2A10v3 and h2A10v4 after a single I.V. dose of 10 mg/kg in a cynomolgus monkey PK study (n=4 per group).

FIG. 18 contains an amino acid sequence alignment of the light chain variable region of rabbit 4A11 mAb and its humanized variants, v1-v8; the sequences shown correspond to SEQ ID NOs: 26, 80, 82, 80, 82, 84, 85, 84, and 84, from top sequence to bottom sequence in the alignment. Residue numbering is according to Kabat; “*” indicates Vernier zone. CDR regions according to Chothia or Kabat (as indicated) are boxed; mutated residues are highlighted.

FIG. 19 contains an amino acid sequence alignment of the heavy chain variable region of the rabbit 4A11 and humanized variants, v1-v8; the sequences shown correspond to SEQ ID NOs: 27, 81, 81, 83, 83, 81, 81, 83, and 83 (from top sequence to bottom sequence in the alignment). Residue numbering is according to Kabat; “*” indicates Vernier zone. CDR regions according to Chothia or Kabat (as indicated) are boxed; mutated residues are highlighted.

FIG. 20 contains an amino acid sequence alignment of the light chain variable regions of the humanized 4A11 variants v7-v7.19; the sequences shown correspond to SEQ ID NOs: 84, 84, 84, 84, 89-92, 101, 101, 101, and 101 (from top sequence to bottom sequence in the alignment). Residue numbering is according to Kabat; “*” indicates Vernier zone. CDR regions according to Chothia or Kabat (as indicated) are boxed; mutated residues are highlighted.

FIG. 21 contains an amino acid sequence alignment of the heavy chain variable region of the humanized 4A11 variants v7-v7.19; the sequences shown correspond to SEQ ID NOs: 83, 86, 87, 88, 83, 83, 83, 83, 93-100, and 102-105 (from top sequence to bottom sequence in the alignment). Residue numbering is according to Kabat; “*” indicates Vernier zone. CDR regions according to Chothia or Kabat (as indicated) are boxed; mutated residues are highlighted.

FIG. 22 contains amino acid sequence alignments of the light chain (upper panel) and heavy chain (lower panel) variable region sequences of the rabbit 6F12 antibody and humanized variants v1-v4; the amino acid sequences shown in the figure are SEQ ID NOs: 24, 131, 131, 143, and 144 (upper panel, from top sequence to bottom sequence in the alignment) and SEQ ID NOs: 25, 132, 142, 132, and 142 (lower panel, from top sequence to bottom sequence in the alignment). Residue numbering is according to Kabat; “*” indicates Vernier zone. CDR regions according to Chothia or Kabat (as indicated) are boxed; mutated residues are highlighted.

FIG. 23 contains an amino acid sequence alignment of the light chain variable regions of the humanized 6F12 v1 variants v1-v1.9; the sequences shown correspond to SEQ ID NOs: 131, 133-137, 131, 131, 131, and 131 (from top sequence to bottom sequence in the alignment). Residue numbering is according to Kabat; “*” indicates Vernier zone. CDR regions according to Chothia or Kabat (as indicated) are boxed; mutated residues are highlighted.

FIG. 24 contains an amino acid sequence alignment of the heavy chain variable regions of the humanized 6F12 v1 variants v1-v1.9; the sequences shown correspond to SEQ ID NOs: 132, 132, 132, 132, 132, 132, and 138-141 (from top sequence to bottom sequence in the alignment). Residue numbering is according to Kabat; “*” indicates Vernier zone. CDR regions according to Chothia or Kabat (as indicated) are boxed; mutated residues are highlighted.

FIG. 25 is an image of the crystal structure of 2A10 in complex with human TGFβ3.

FIG. 26 is an image of TGFβ3 bound to 2A10, and comparing to TGFβ1 with TGFBR1/TGFBR2 complex. It indicates that 2A10 would sterically block TGFBR2 recruitment, but likely not TGFBR1.

FIG. 27 is an image of fresolimumab (“freso”), a pan-TGFβ antibody, compared with 2A10. Fresolimumab blocks TGFβ3 binding to both TGFBR1/TGFBR2, but 2A10 only blocks binding to TGFBR2 due to a different binding angle.

FIG. 28 shows the epitope on TGFβ3 bound by 2A10. 2A10 binds the beta-hairpin at the tip of beta 6-beta7 “finger” of TGFβ3 through R394. Polar contacts are also made between R325 and K331.

FIG. 29 shows the epitope on TGFβ3 bound by 2A10; residues involved in binding of TGFβ3 by 2A10 within 5.0 Å are labeled.

FIG. 30 shows the 2A10 antibody paratope (residue numbering according to Kabat). R394 on TGFβ3 is in contact with D50 of 2A10.

FIG. 31 shows the TGFβ3/2A10 epitope and a comparison to TGFβ1. TGOF1 has three changes in epitopic residues, when compared to TGFβ3. The P387T, L389V and T395K substitutions may subtly change the conformation of the beta-hairpin and β6-β7 fingers of TGFβ2 and remove some beneficial contacts with 2A10.

FIG. 32 shows the TGFβ3/2A10 epitope and a comparison to TGFβ2. TGFβ2 has four changes in epitopic residues when compared to TGFβ3. The primary reasons for TGFβ3 vs. TGFβ2 specificity are likely to be based on the R394K change, which removes an optimized salt bridge in epitope R394-D50, and V398I, adding a potential steric clash.

FIG. 33 shows a comparison of TGFβ2 binding to 4A11v2 (left cartoon) and 4A11v7 (right cartoon). The antigen-antibody complex is very similar. The heavy chains come close to each other upon binding, but the binding elbow angle of the second Fab is different.

FIG. 34 shows a comparison of 4A11v2/TGFβ2 to the TGFβ3/receptor complex. 4A11v2 does not directly compete with the TGFBR1/2 binding sites; rather, 4A11v2 slightly alters structure of TGFβ2, causing the fingers to “pinch” together. This may be a result of a steric clash between 4A11v2 Fabs.

FIG. 35 shows the TGFβ2 epitope bound by 4A11. Glutamic acid at position E373 of the TGFβ2 amino acid sequence (TGFβ2 numbering) is at the center of the contact interface. TGFβ3 also has glutamic acid at this position, similar to TGFβ2, whereas TGFβ1 has a glycine.

FIG. 36 contains cartoons showing the conformational changes in TGFβ2 induced by 4A11v2 (right cartoon) and the TGFβ2 from fresolimumab (“freso”) complex (left cartoon). Conformational changes in TGFβ2 structure may alter the ability of the signaling receptors to bind TGFβ2. 4A11 may also block access of the membrane tethered receptors, by binding the membrane proximal side of TGFβ2 or TGFβ3.

FIG. 37 is a cartoon showing TGFβ2 from the 4A11v2 complex (left image) and TGFβ3 from TGFβR1/TGFβR2 complex. Conformation changes in the TGFβ2 structure may alter the ability of TGFBR1 to bind on one monomer.

FIG. 38 shows the interaction of heavy chain of 4A11v2 (left structure) and 4A11v7 (right structure) residues; VH framework interaction are complementary in both structures.

FIG. 39 shows 4A11v2 and 4A11v7 VH framework comparisons. In 4A11v2, Q81 is packed against R19 in a high-energy rotamer and awkward position. In 4A11v7, K81 forms a weak H bond to S79, therefore framework residues in v2 are likely repulsive, while v7 residues are more permissive.

FIG. 40 contains cartoons illustrating monovalent (left cartoon) and divalent (right cartoon) 4A11 binding to TGFβ2.

FIG. 41 is a graph plotting the relative luciferase activity of MLEC reporter cells following coculture with 293T cells transfected with a plasmid encoding FL-TGFβ1 or FL-TGFβ3 (wild-type or RGE mutants) with or without plasmids encoding integrins av and 06. **, P<0.01; ***, P<10⁻³; ****, P<10⁻⁴; NS, P>0.05 by unpaired two-tailed Student's t-test.

FIG. 42 is a graph plotting the relative luciferase activity in supernatants (SN) from MLEC reporter cells (measuring TGFβ activity). SN were collected from cells transfected with a plasmid encoding FL-TGFβ1, FL-TGFβ2, FL-TGFβ3 or an empty vector (control). Supernatants were acidified as indicated (HCl treatment). **, P<0.01; ***, P<10⁻³; ****, P<10⁻⁴; NS, P>0.05 by unpaired two-tailed Student's t-test.

FIG. 43A and FIG. 43B are graphs plotting the TGFβ activity assessed by alkaline phosphatase released by HEK-Blue TGFβ reporter cells (FIG. 43A) or with MLEC reporter cell assay (relative luciferase activity) (FIG. 43B). In FIG. 43A, supernatants (SN) were collected from 293T cells transfected with a plasmid encoding FL-TGFβ1, FL-TGFβ2, FL-TGFβ3 or an empty vector (control). In FIG. 43B, SN were collected from COS-7 cells transfected with a plasmid encoding FL-TGFβ1, FL-TGFβ3 or an empty vector (control). **, P<0.01; ***, P<0.001; ****, P<10⁻⁴; NS, P>0.05 by unpaired two-tailed Student's t-test. Data are averages ±SEM from experiments run in triplicate. Error bars are not shown when they are shorter than sizes of their corresponding symbols.

FIG. 44 is a graph plotting the TGFβ activity assessed by MLEC reporter cell assay (relative luciferase activity). 293T cells were transfected with plasmids encoding various forms of FL-TGFβ and integrins av and β6. TGFβ activities were measured with MLEC reporter cells after co-culture. Cytochalasin D (30 μM) was added at the beginning of the co-culture where indicated (“Cyto D”). “MILE” is control. **, P<0.01; ***, P<0.001; ****, P<10⁻⁴; NS, P>0.05 by unpaired two-tailed Student's t-test.

FIG. 45 is a graph plotting the TGFβ activity assessed by MLEC reporter cell assay (relative luciferase activity) in supernatants collected from 293T cells transfected with plasmids encoding wild-type or furin site mutant FL-TGFβ proteins, showing the loss of TGFβ activities with the mutated proteins. **, P<0.01; ***, P<0.001; ****, P<10⁻⁴; NS, P>0.05 by unpaired two-tailed Student's t-test. Data are averages ±SEM from experiments run in triplicate. Error bars are not shown when they are shorter than sizes of their corresponding symbols.

FIG. 46 is an image of a Western blot showing the kinetics of smad2 phosphorylation in NHLF cells treated with recombinant proteins or supernatants. As controls, NHLF cells were also treated with supernatant from non-transfected cells (SN.ctrl) or the recombinant BMP2 for 1 hour. To control the loadings among different samples, protein concentrations of supernatants were measured by BCA and 20 μg protein was loaded in each lane.

FIG. 47 is a graph plotting TGFβ activity measured in MLEC reporter cells (relative luciferase activity) after incubation with a series of concentrations of human Fc-FL-TGFβ isoform fusion proteins. “Background” means no fusion protein was added. Data are averages ±SEM from experiments run in triplicate and error bars are not shown when they are shorter than sizes of their corresponding symbols.

FIG. 48 is a graph plotting TGFβ activity measured in MLEC reporter cells (relative luciferase activity) after incubation with human Fc-FL-TGFβ2 or Fc-FL-TGFβ3 fusion proteins (30 ng/ml) and isoform specific antibodies: 19D8 (anti-TGFβ1); 6F12 (anti-TGFβ2); 2A10 (anti-TGFβ3); and 1D11 (pan-anti-TGFβ) at 3 μg/ml. “Background” means no fusion protein was added. “None” means no antibody added. Data are averages ±SEM from experiments run in triplicate and error bars are not shown when they are shorter than sizes of their corresponding symbols.

FIG. 49 is a table showing the primary amino acid sequence similarities (%) among human TGFβ isoforms of the latency associated peptides (LAPs) and mature domains (active).

FIG. 50A, FIG. 50B, and FIG. 50C are graphs showing titration curves from MLEC reporter cell assay (TGFβ activity) following incubation of mature peptide (1 ng/ml) of TGFβ1, TGFβ2 or TGFβ3 (left to right graphs) with a series of concentrations of human Fc-LAP fusion proteins as indicated. Data are averages ±SEM from experiments run in triplicate and error bars are not shown when they are shorter than sizes of their corresponding symbols.

FIG. 51 is a table showing the IC50s based on the titration curves shown in FIG. 50.

FIG. 52A and FIG. 52B are graphs showing percentages (%) of new hydroxyproline (mean±SEM) measured as an indicator of newly synthesized collagen in WT or TGFβ isoform CKO mice. In FIG. 52A, n=5 (each saline group), n=20 (WT, bleomycin treated (“BLM”)), n=24 mice (β2.cKO, BLM); in FIG. 52B, n=7 (WT, saline), n=14 (WT, BLM), n=13 (β3.cKO, BLM) and n=13 (β2/3.cDKO, BLM); *P<0.05, ***P<0.001 by One-way ANOVA with Dunnett's test.

FIG. 53A and FIG. 53B are bar graphs plotting whole lung gene expression of Serpine1, Fn1 and Col1a1 as determined by quantitative RT-PCR 14 days after I.T. saline or bleomycin instillation and treatment with isotype control antibody, anti-TGFβ2 antibody (6F12) (FIG. 53A) or anti-TGFβ3 antibody (2A10) (FIG. 53B). In FIG. 53A, n=5 (Saline), n=15 (control, bleomcyin (“BLM”)) and n=14 (6F12, BLM); In FIG. 53B, n=5 (Saline), n=19 (control, BLM) and n=20 mice (2A10, BLM). *P<0.05, **P<0.01, *** P<0.001, **** P<0.0001, NS, P>0.05 by One-way ANOVA with Dunnett's test.

FIG. 53C is a bar graph plotting the newly synthesized collagen levels as determined at day 24 after saline (n=10) or bleomycin (“BLM”) installation. Animals were treated with either an isotype control (n=21), the combination (n=23) of 6F12 and 2A10, or anti-TGFβ2/3 antibody (4A11). Dose levels of 4A11 were high (10 mg/kg; 3 times/week, n=23); middle (10 mg/kg; 1 time/week, n=24), or the low (2.5 mg/kg; 1 time/week, n=23), respectively. *P<0.05, **P<0.01, *** P<0.001, **** P<0.0001, NS, P>0.05 by One-way ANOVA with Dunnett's test.

FIG. 53D is a bar graph plotting the disease scores as determined by pathology analyses. Mouse lungs were harvested 24 days after either saline (n=5) or bleomycin (“BLM”) instillation, with treatment by either isotype control (n=7) or 4A11 (high dose, n=12). *P<0.05, **P<0.01, *** P<0.001, **** P<0.0001, NS, P>0.05 by One-way ANOVA with Dunnett's test.

FIG. 53E contains representative mouse lung images of immunohistochemistry staining for collagen III.

FIG. 54A contains bar graphs plotting relative gene expression levels of TGFβ isoforms in livers from NASH patients with mild fibrosis (F0 and F1) (N=40) vs. severe fibrosis (F3 and F4) (N=32).

FIG. 54B contains dot plots showing relative gene expression levels of TGFβ isoforms in mouse livers as determined by quantitative RT-PCR at 6 weeks after the initiation of vehicle or CCl₄ treatment; n=8 for both groups.

FIG. 54C contains bar graphs plotting the liver gene expression of Col1a1 and Col3a1 as determined by quantitative RT-PCR 6 weeks after the initiation of vehicle or CCl₄ treatment; animals were preventively treated with either isotype control or 4A11; n=8 for all groups. *, P<0.05; **, P<0.01; ****, P<10⁻⁴; NS, P>0.05 by unpaired two-tailed Student's t-test (A and B); **P<0.01 by One-way ANOVA with Dunnett's test.

FIG. 54D is a bar graph plotting the liver pathology scores as determined by histopathological analysis; mouse livers were harvested 6 weeks after either vehicle or CCl₄ treatment; n=8 for all groups. *, P<0.05; **, P<0.01; ****, P<10⁻⁴; NS, P>0.05 by unpaired two-tailed Student's t-test (A and B); **P<0.01 by One-way ANOVA with Dunnett's test.

FIG. 55A is a table showing the binding affinity (K_{D}) of antibodies 6F12 and 4A11 for human and mouse TGFβ2 determined by Biacore SPR.

FIG. 55B contains line graphs plotting TGFβ activities as measured with MLEC reporter cells. Mature peptide (1 ng/ml) of human or mouse TGFβ2 was incubated with a series of concentrations of 6F12 or 4A11 antibodies. Curves are best fit to dose-response inhibition model. BKGD (background), no mature peptide added.

FIG. 56A is a schematic for a colitis model to assess the enhanced inflammatory responses associated with anti-TGFβ antibodies.

FIG. 56B provides colon weights (grams) measured at day 24 in the colitis model of FIG. 56A. N=10 for all groups except the untreated group (n=6).

FIG. 56C provides the relative expression of inflammatory genes (analyzed from colon RNA) in the colitis model of FIG. 56A. Three to five mice from each group were analyzed as indicated.

FIG. 56D summarizes the immune cell numbers in lamina propria from the remaining mice in each group (3-5/group), as determined by flow cytometry using their surface markers, in the colitis model of FIG. 56A.

### DETAILED DESCRIPTION OF EMBODIMENTS OF THE INVENTION

### I. Definitions

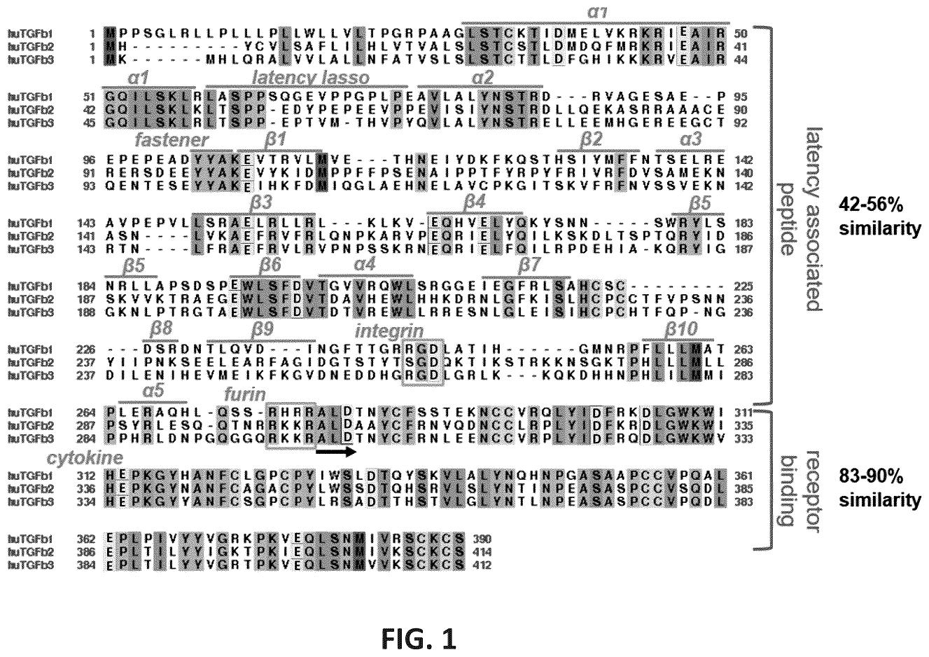

As used herein, the terms “tumor necrosis factor β” and “TGFβ” are used interchangeably and refer to any native TGFβ isoform from any vertebrate source, including mammals such as primates (e.g. humans) and rodents (e.g., mice and rats), unless otherwise indicated. The term encompasses “full-length,” unprocessed TGFβ as well as any form of TGFβ that results from processing in the cell. The term also encompasses naturally occurring variants of TGFβ, e.g., splice variants or allelic variants. TGFβ isoforms have precursor forms (immature) and mature forms. The latency-associated peptide, as shown in FIG. 1, is cleaved by furin proteases intracellularly and forms a non-covalent interaction with the receptor-binding domain. This complex is secreted from the cell, alone or covalently bound via the latency-associated peptide to ‘milieu’ molecules such as GARP, LRRC33, or latent TGFβ-binding proteins (LTBP)1-4. The complex of LAP and the receptor-binding domain is termed the small latent complex (SLC) and the complex of the LAP, the receptor-binding domain, and a milieu molecule is termed the large latent complex (LLC).

Amino acid sequences for TGFβ1, TGFβ2, and TGFβ3 are as follows:

Human TGFβ1
(SEQ ID NO: 1)
MPPSGLRLLPLLLPLLWLLVLTPGRPAAGLSTCKTIDMELVKRKRIEAI
RGQILSKLRLASPPSQGEVPPGPLPEAVLALYNSTRDRVAGESAEPEPE
PEADYYAKEVTRVLMVETHNEIYDKFKQSTHSIYMFFNTSELREAVPEP
VLLSRAELRLLRLKLKVEQHVELYQKYSNNSWRYLSNRLLAPSDSPEWL
SFDVTGVVRQWLSRGGEIEGFRLSAHCSCDSRDNTLQVDINGFTTGRRG
DLATIHGMNRPFLLLMATPLERAQHLQSSRHRRALDTNYCFSSTEKNCC
VRQLYIDFRKDLGWKWIHEPKGYHANFCLGPCPYIWSLDTQYSKVLALY
NQHNPGASAAPCCVPQALEPLPIVYYVGRKPKVEQLSNMIVRSCKCS.
Human TGFβ2
(SEQ ID NO: 2)
MHYCVLSAFLILHLVTVALSLSTCSTLDMDQFMRKRIEAIRGQILSKLK
LTSPPEDYPEPEEVPPEVISIYNSTRDLLQEKASRRAAACERERSDEEY
YAKEVYKIDMPPFFPSENAIPPTFYRPYFRIVRFDVSAMEKNASNLVKA
EFRVFRLQNPKARVPEQRIELYQILKSKDLTSPTQRYIDSKVVKTRAEG
EWLSFDVTDAVHEWLHHKDRNLGFKISLHCPCCTFVPSNNYIIPNKSEE
LEARFAGIDGTSTYTSGDQKTIKSTRKKNSGKTPHLLLMLLPSYRLESQ
QTNRRKKRALDAAYCFRNVQDNCCLRPLYIDFKRDLGWKWIHEPKGYNA
NFCAGACPYLWSSDTQHSRVLSLYNTINPEASASPCCVSQDLEPLTILY
YIGKTPKIEQLSNMIVKSCKCS.
Human TGFβ3
(SEQ ID NO: 3)
MKMHLQRALVVLALLNFATVSLSLSTCTTLDFGHIKKKRVEAIRGQILS
KLRLTSPPEPTVMTHVPYQVLALYNSTRELLEEMHGEREEGCTQENTES
EYYAKEIHKFDMIQGLAEHNELAVCPKGITSKVFRFNVSSVEKNRTNLF
RAEFRVLRVPNPSSKRNEQRIELFQILRPDEHIAKQRYIGGKNLPTRGT
AEWLSFDVTDTVREWLLRRESNLGLEISIHCPCHTFQPNGDILENIHEV
MEIKFKGVDNEDDHGRGDLGRLKKQKDHHNPHLILMMIPPHRLDNPGQG
GQRKKRALDTNYCFRNLEENCCVRPLYIDFRQDLGWKWVHEPKGYYANF
CSGPCPYLRSADTTHSTVLGLYNTLNPEASASPCCVPQDLEPLTILYYV
GRTPKVEQLSNMVVKSCKCS.

The term “TGFβ1” as used herein, refers to any native TGFβ1 from any vertebrate source, including mammals such as primates (e.g., humans) and rodents (e.g., mice and rats), unless otherwise indicated. The term encompasses “full-length,” unprocessed TGFβ1 as well as any form of TGFβ1 that results from processing in the cell. The term also encompasses naturally occurring variants of TGFβ1, e.g., splice variants or allelic variants. The amino acid sequence of an exemplary human TGFβ1 is shown in FIG. 1 (SEQ ID NO: 1).

The term “TGFβ2” as used herein, refers to any native TGFβ2 from any vertebrate source, including mammals such as primates (e.g., humans) and rodents (e.g., mice and rats), unless otherwise indicated. The term encompasses “full-length,” unprocessed TGFβ2 as well as any form of TGFβ2 that results from processing in the cell. The term also encompasses naturally occurring variants of TGFβ2, e.g., splice variants or allelic variants. The amino acid sequence of an exemplary human TGFβ2 is shown in in FIG. 1 (SEQ ID NO: 2).

The term “TGFβ3” as used herein, refers to any native TGFβ3 from any vertebrate source, including mammals such as primates (e.g., humans) and rodents (e.g., mice and rats), unless otherwise indicated. The term encompasses “full-length,” unprocessed TGFβ3 as well as any form of TGFβ3 that results from processing in the cell. The term also encompasses naturally occurring variants of TGFβ3, e.g., splice variants or allelic variants. The amino acid sequence of an exemplary human TGFβ3 is shown in in FIG. 1 (SEQ ID NO: 3).

As used herein, the term “TGFβ” means any one, two or all three of the TGFβ isoforms TGFβ1, TGFβ2 and TGFβ3, as described above.

As used herein, the terms “specifically binds” and “binds specifically to” refer to an antibody selectively or preferentially binding to its target antigen. Preferably the binding affinity for antigen is of K_{D} value of 10⁻⁹ mol/1 or lower (e.g. 10⁻¹⁰ mol/l), preferably with a K_{D} value of 10⁻¹ mol/1 or lower (e.g., 10⁻¹² mol/l). The binding affinity is determined with a standard binding assay, such as surface plasmon resonance technique (BIACORE®).

As used herein, the term “anti-TGFβ antibody” means an antibody that specifically binds to one or more TGFβ isoform(s). Thus, as used herein, the term “anti-TGFβ3 antibody” refers to a monospecific antibody that specifically binds to TGFβ3; the term “anti-TGFβ2 antibody” refers to a monospecific antibody that specifically binds to TGFβ2 (e.g., human TGFβ2); the term “anti-TGFβ2/3 antibody” refers to a dual-specific antibody that specifically binds to TGFβ2 (e.g., human TGFβ2) and TGFβ3 (e.g., human TGFβ3); the term “anti-TGFβ1 antibody” refers to a monospecific antibody that specifically binds to TGFβ1 (e.g., human TGFβ1); and the term “pan-specific TGFβ antibody” refers to an antibody that binds to all three TGFβ isoforms (TGFβ1, TGFβ2, and TGFβ3, e.g., human TGFβ1, TGFβ2 and TGFβ3)). An anti-TGFβ antibody described herein that is mono- or dual-specific for certain TGFβ isoform(s) is also referred to herein as an “isoform-selective anti-TGFβ antibody.” In one embodiment, the extent of binding of an isoform-selective anti-TGFβ antibody (e.g., an anti-TGFβ1 antibody, an anti-TGFβ2 antibody, an anti-TGFβ2/3 antibody, or an anti-TGFβ3 antibody) to the TGFβ isoform(s) for which the antibody is not specific is less than about 10% of the binding of the antibody to its target TGFβ isoform(s), as measured, e.g., by a radioimmunoassay (RIA) or surface plasmon resonance (SPR). In another embodiment, the extent of binding of an isoform-selective anti-TGFβ antibody to the TGFβ isoform(s) for which the antibody is not specific is less than about 9%, 8%, 7%, 6%, 5%, 4%, 3%, 2%, or preferably less than about 1% of the binding of the antibody to its target TGFβ isoform(s), as measured, e.g., by RIA or SPR. In one embodiment, an isoform-selective anti-TGFβ antibody refers to an antibody that is capable of binding the TGFβ isoform(s) for which the antibody is specific with sufficient affinity such that the antibody is useful as a diagnostic and/or therapeutic agent in targeting the TGFβ isoform(s). In one embodiment, the extent of binding of an isoform-selective anti-TGFβ antibody to an unrelated protein is less than about 10%, or less than about 9%, 8%, 7%, 6%, 5%, 4%, 3%, 2%, or preferably less than about 1% of the binding of the isoform-selective anti-TGFβ antibody as measured, e.g., by a radioimmunoassay (RIA). In certain embodiments, an anti-TGFβ antibody has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸M or less, e.g., from 10⁻⁸ M to 10⁻¹³ M, e.g., from 10⁻⁹ M to 10⁻¹³ M) to its target TGFβ isoform(s). In a preferred embodiment, an isoform-selective anti-TGFβ antibody has a K_{D} of ≤250 μM.

As used herein, the term “selectively neutralize” and its grammatical variations, with respect to an isoform-selective anti-TGFβ antibody described herein, means that the antibody specifically binds to and neutralizes the isoform(s) for which the antibody is selective but does not neutralize the other isoform(s). Thus, for example, an anti-TGFβ1 antibody that selectively neutralizes TGFβ1 does not neutralize TGFβ2 or TGFβ3; an anti-TGFβ2 antibody that selectively neutralizes TGFβ2 does not neutralize TGFβ1 or TGFβ3; an anti-TGFβ3 antibody that selectively neutralizes TGFβ3 does not neutralize TGFβ1 or TGFβ2; an anti-TGFβ2/3 antibody that selectively neutralizes TGFβ2 and TGFβ3 does not neutralize TGFβ1. In certain embodiments, an isoform-selective anti-TGFβ antibody as described herein specifically binds to an epitope of a TGFβ isoform that is conserved across different species. In some embodiments, the ability of an antibody (e.g., an isoform-selective anti-TGFβ antibody described herein) to selectively neutralize one or more TGFβ isoforms may be determined in an in vitro inhibition assay, e.g., the cell-based inhibition TGFβ assay described herein below.

As used herein, a “TGFBR” means a TGFβ receptor. Dimeric receptor-binding domains of all three TGFβ isoforms bind to pairs of heterodimeric receptor complexes of TGFBR1 and TGFBR2; the tetrameric receptor complex then activates intracellular signaling via receptor tyrosine kinase (RTK) activity of TGFBR1, also known as ALK5 (Weiss et al., Wiley Interdiscip. Rev. Dev. Biol. 2:47-63 (2013)). In canonical TGFBR signaling, ALK5 phosphorylates SMAD2 and SMAD3, which then associate with SMAD4, translocate to the nucleus, and direct gene transcription. The Smad complex activates transcription of myofibroblast genes including αSMA, calponin and collagen (Usuki et al., J. Nippon Med. Sch. 79:46-59 (2012); Carthy et al., PloS one 6:e19809 (2011); and Gu et al., Acta Pharmacol. Sin. 28:382-391 (2007)). There are additional non-SMAD dependent signaling pathways that may be activated by TGFβ under certain contexts including MAP kinases, AKT, JAK-STAT, and NFκB. Biochemical and structural studies have shown that the assembly of TGFβ-TGFβR signaling complexes may have subtle isoform-specific differences: TGFβ1 and TGFβ3 bind more strongly to TGFβR2 and only form strong interactions with TGFβR1 when in complex with TGFβR2, whereas TGFβ2 binds weakly to both TGFBR1 and TGFBR2 alone, and avidity may drive full complex formation (Radaev J B C 2009; 285, 14806-14814). TGFβ1 and TGFβ2 crystallize in “closed” structures that facilitate binding to TGFBR1 and TGFBR2, while TGFβ3 can adopt a similar “closed” or less ordered “open” structure in crystal form, which may lead to differences in the avidity of ligand-receptor complex assembly (Hinck, A. P. et al. 2016; Cold Spring Harb Perspect Biol doi: 10.1101/cshperspect.a02210). In addition, TGFBR3 (betaglycan), a non-signaling receptor, can facilitate TGFβ2 binding to TGFβR1/2 complexes, but does not appear to play a similar role in TGFβ1 or TGFβ3 receptor binding (del Re, JBC 2004; 279, 22765-22772). While in most cells (including endothelial cells) TGFβ signals through TGFBR1/2 complexes, in endothelial cells TGFβ1 and TGFβ3 can also signal through a TGFBR2/ALK1 complex facilitated by endoglin, which contributes to SMAD1/5 dependent vascular endothelial proliferation and angiogenesis (EMBO J. 2004 Oct. 13; 23(20):4018-28). Ultimately, despite these differences in signaling complex assembly, recombinant receptor-binding domains of TGFβ1, 2, and 3 are all able to induce TGFBR-dependent SMAD signaling to a similar degree in cell-based in vitro assays. Hence, any biological differences in the activity of endogenous TGFβ isoforms are more likely due to differences in their patterns of expression and mechanisms of release from SLC or LLC than to differences in their receptor-binding domains.

As used herein, the term “neutralize” and its grammatical variations, with respect to an isoform-selective anti-TGFβ antibody described herein, means that the antibody inhibits to a measurable extent its target TGFβ isoform(s) from inducing signaling through a TGFBR complex.

As used herein, the term “directly contacts” and its grammatical variations, with respect to an antigen-binding domain of an anti-TGFβ antibody, means that the antigen-binding domain is within 15-8, 8, 8-5, or preferably within 5 angstroms of the residue of an amino acid in its corresponding epitope.

“TGFβ disorders” or “TGFβ-related disorders” refers to any disorder, disease, or condition that would benefit from treatment with an isoform-selective anti-TGFβ antibody provided herein. This includes chronic and acute disorders or diseases including those pathological conditions that predispose the mammal to the disorder in question. Disorders to be treated herein include diseases characterized by accumulation of extracellular matrix, diseases caused by circulating TGFβ or TGFβ activated at a local site, including one or more TGFβ isoforms, conditions caused by suppression of the immune system due to endogenous TGFβ production, acute immune deficiencies resulting from severe injuries, burns, and illnesses such as viral or bacterial infections, multi-organ systemic illnesses due to TGFβ production or overproduction, and TGFβ-producing tumors.

As used herein, the terms “fibrosis,” “fibrosis conditions,” and “fibrotic conditions” are intended to have the same meaning. In certain embodiments, the fibrotic conditions are those mediated by a fibrotic stimulator. Exemplary fibrotic stimulators include, without limitation, TGFβ, endothelin, lactic acid (via lactate dehydrogenase), IL-1, Thy-1 (CD90), connective tissue growth factor (“CTGF”), as well as combinations thereof. In certain embodiments, the fibrotic condition is one that is mediated by TGFβ. In certain embodiments, the fibrotic condition is one that is mediated by one or more of TGFβ1, TGFβ2, and TGFβ3. In certain embodiments, the fibrotic condition is one that is mediated by one or both of TGFβ2 and TGFβ3. In certain embodiments, the fibrotic condition is one that is mediated by TGFβ2. In certain embodiments, the fibrotic condition is one that is mediated by TGFβ3. Exemplary fibrotic conditions are described in more detail herein, below.

“Systemic sclerosis” (SSc) or “scleroderma” is a complex and heterogeneous disease with skin and tissue fibrosis, vascular alterations, and autoantibodies against various cellular antigens being amongst its principal features. The clinical manifestations of systemic sclerosis can range from limited skin involvement to severe internal organ dysfunction. Internal visceral organ pathology is a major factor contributing to the morbidity of this disease, with the kidneys, esophagus, heart, and lungs being the most frequently involved. There are two major subgroups in the commonly accepted classification of SSc: limited cutaneous SSc (IcSSc) and diffuse cutaneous SSc (dcSSc). Gabrielli et al. Mechanisms of disease. Scleroderma. N Engl J Med 360:1989-2003 (2009). In one embodiment, the patient with systemic sclerosis has been classified according to the American College of Rheumatology (formerly, the American Rheumatism Association) criteria for the classification of systemic scleroderma based on: major criterion: diffuse (truncal) sclerosis (skin tightness, thickening, and non-pitting induration); and minor criteria: (1) sclerodactyly (only fingers and/or toes), (2) digital pitting scars or loss of substance of the digital finger pads (pulp loss), and (3) bilateral basilar pulmonary fibrosis, wherein a patient with systemic sclerosis should fulfill the major criterion or two of the three minor criteria. See Subcommittee for Scleroderma Criteria of the American Rheumatism Association, Diagnostic and Therapeutic Criteria Committee. Preliminary criteria for the classification of systemic sclerosis (scleroderma). Arthritis Rheum 23:581-90 (1980).

As used herein, Chronic Obstructive Pulmonary Disease (“COPD”) is an umbrella term used to describe a group of respiratory tract diseases generally characterized by airflow obstruction or limitation. This condition may also be known under the terms chronic obstructive respiratory disease (CORD), chronic obstructive airways disease (COAD), chronic obstructive lung disease (COLD), or chronic airway limitation (CAL). As used herein, the term COPD is intended to encompass all such references. The clinical course of COPD is characterized by chronic disability, with intermittent, acute exacerbations that occur more often during the winter months. An acute exacerbation of COPD can be defined as a sustained worsening of the patient's symptoms from his or her usual stable state that is beyond normal day-to-day variations, and is acute in onset. When acute exacerbations occur, they typically manifest as increased sputum production, more purulent sputum, change in sputum color, increased coughing, upper airway symptoms (e.g., colds and sore throats), increased wheezing, chest tightness, reduced exercise tolerance, increased fatigue, fluid retention, acute confusion, and worsening of dyspnea. Although infectious etiologies account for most exacerbations, exposure to allergens, pollutants, or inhaled irritants may also play a role. Infectious agents known to cause acute exacerbations of COPD include: rhinoviruses, influenza, parainfluenza, coronavirus, adenovirus, respiratory syncytial virus, Chlamydia pneumoniae, Haemophilus influenzae, Streptococcus pneumoniae, Moraxella catarrhalis, Staphylococcus aureus, Mycloplasma pneumoniae, and Pseudomonas aeruginosa. Pollutants known to cause acute exacerbations include nitrogen dioxide, particulates, sulfur dioxide, and ozone. Despite these known causes, the exact cause of exacerbations may be unidentifiable in up to 30% of diagnosed cases of exacerbation of COPD. The Global Initiative for Chronic Obstructive Lung Disease (GOLD) defines COPD as a disease state characterized by airflow limitation that is not fully reversible. The airflow limitation is usually progressive and associated with abnormal inflammatory response of the lungs to noxious particles or gases. The American Thoracic Society (ATS) defines COPD as a disease process involving progressive chronic airflow obstruction because of chronic bronchitis, emphysema, or both. Chronic bronchitis is defined clinically as excessive cough and sputum production on most days for at least three months during at least two consecutive years. Emphysema is characterized by chronic dyspnea (shortness of breath) resulting from the destruction of lung tissue and the enlargement of air spaces. A further condition typically encompassed by the term COPD is bronchiectasis, which is an abnormal stretching and enlarging of the respiratory passages caused by mucus accumulation and blockage. Under such conditions, the weakened passages can become scarred and deformed, allowing more mucus and bacteria to accumulate, resulting in a cycle of infection and blocked airways.

As used herein, the term “ILD” refers to interstitial lung disease. Interstitial lung diseases include a large and diverse group of more than 200 lung diseases and respiratory conditions characterized by inflammation and fibrosis of the interstitium, the tissue and space between the air sacs of the lung (see, for instance, du Bois, Nat. Rev. Drug Discov. 2010, 9, 129-140). In “Progressive Fibrosing Interstitial Lung Diseases (PF-ILD)” the response to lung injury in fibrosing ILDs includes the development of fibrosis which becomes progressive, self-sustaining and independent of the original clinical association or trigger.

As used herein, the terms “idiopathic pulmonary fibrosis” and “IPF” refer to a restrictive lung disease characterized by progressive interstitial fibrosis of lung parenchyma, affecting approximately 100,000 patients in the United States (Raghu et al., Am J Respir Crit Care Med 174:810-816 (2006)). This interstitial fibrosis associated with IPF leads to progressive loss of lung function, resulting in death due to respiratory failure in most patients. The median survival from the time of diagnosis is 2-3 years (Raghu et al., Am J Respir Crit Care Med 183:788-824 (2011)). The etiology and key molecular and pathophysiological drivers of IPF are unknown. In some embodiments, a diagnosis of IPF is confirmed by the finding of usual interstitial pneumonia (UIP) on histopathological evaluation of lung tissue obtained by surgical biopsy. The criteria for a diagnosis of IPF are known. Ryu et al. (1998) Mayo Clin. Proc. 73:1085-1101.

As used herein, “GI tract fibrosis” refers to fibrosis of the gastrointestinal tract, including, e.g., the mouth, esophagus, stomach, small intestine, large intestine, and anus. “GI tract fibrosis” thus includes intestinal fibrosis. “Intestinal fibrosis” is a common complication of inflammatory bowel disease (IBD), and is usually defined as an excessive accumulation of scar tissue in the intestinal wall. Intestinal fibrosis can occur in both forms of IBD: ulcerative colitis and Crohn's disease. “GI tract fibrosis” includes, but is not limited to, fibrosis associated with Crohn's disease, ulcerative colitis, collagenous colitis, colorectal fibrosis, villous atrophy, crypt hyperplasia, polyp formation, healing gastric ulcer, and microscopic colitis.

As used herein “monitoring disease progression” refers to assessing a subject (e.g., a subject suffering from a TGFβ-related disorder, e.g. a subject undergoing treatment with an anti-TGFβ antibody as described elsewhere herein) at successive time intervals to determine whether disease symptoms have worsened, stabilized, or improved (i.e., become less severe). For example, monitoring the progression of fibrosis (e.g., SSc or IPF, or other fibrosis) in a subject can, in certain instances, include monitoring changes in the 18-gene TGFβ signature set described in Table 2, below, overall response rate, duration of response, quality of life, expression and/or activity of disease markers (e.g., expression of certain other genes and/or proteins), or other criteria known in the art. Additional approaches to monitoring disease progression in a patient with a TGFβ-related disorder can be employed, including for example, measurement of response to treatment via imaging techniques, which are described in further detail elsewhere herein.

As used herein, the terms “monitoring treatment progress” or “monitoring response to treatment” are used interchangeably and refer to assessing a subject (e.g., a subject suffering from a TGFβ-related disorder, e.g., a subject undergoing treatment with an anti-TGFβ antibody as described elsewhere herein) at successive time intervals during or following treatment to determine whether disease symptoms have worsened, stabilized, or improved (i.e., become less severe) as a result of the treatment. For example, treatment progress in a subject (e.g., a subject who has or is receiving treatment with an immunotherapeutic agent, such as but not limited to an anti-TGFβ antibody described herein) can be monitored using the same criteria as those used to monitor disease progression.

As used herein, the term “detection” includes any means of detecting, including direct and indirect detection.

As used herein, the term “diagnosis” is used herein to refer to the identification or classification of a molecular or pathological state, disease or condition. For example, “diagnosis” may refer to identification of a particular type of fibrosis (e.g., SSc, IPF, etc.) or other TGFβ-mediated disorder. “Diagnosis” may also refer to the classification of a particular subtype of a fibrotic condition, e.g., by histopathological or radiographic criteria or by molecular features (e.g., a subtype characterized by expression of one or a combination of particular genes or proteins encoded by said genes).

The term “prognosis” is used herein to refer to the prediction of the likelihood of survival over time as well as one or more TGFβ-attributable disease symptoms worsening over time.

As used herein, a “control subject” refers to a healthy subject who has not been diagnosed as having the disease or condition of interest, e.g., fibrosis, e.g., IPF, SSc, etc., and who does not suffer from any sign or symptom associated with the disease or condition.

The term “sample,” as used herein, refers to a composition that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized.

By “tissue” or “cell sample” is meant a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like. A “reference sample”, “reference cell”, “reference tissue”, “control sample”, “control cell”, or “control tissue”, as used herein, refers to a sample, cell or tissue obtained from a source known, or believed, not to be afflicted with the disease or condition for which a method or composition of the invention is being used to identify. In one embodiment, a reference sample, reference cell, reference tissue, control sample, control cell, or control tissue is obtained from a healthy part of the body of the same subject or patient in whom a disease or condition is being identified using a composition or method of the invention. In one embodiment, a reference sample, reference cell, reference tissue, control sample, control cell, or control tissue is obtained from a healthy part of the body of an individual who is not the subject or patient in whom a disease or condition is being identified using a composition or method of the invention.

As used herein, the term “gene signature” is used interchangeably with “gene expression signature” and refers to one or a combination of genes whose expression is indicative of a subject or tissue or other sample isolated from a subject having high TGFβ activity and/or indicative of a subject being likely to benefit from treatment with an inhibitor of a TGFβ isoform characterized by certain molecular, pathological, histological, radiographic and/or clinical features. In certain embodiments, the expression of one or more genes comprising the gene signature is elevated compared to that in control subjects.

As used herein, the term “elevated expression level” or “elevated levels” refers to an increased expression of a mRNA or a protein in a subject (e.g., a subject, e.g., a patient, suspected of having or diagnosed as having a TGFβ-related disorder, e.g., fibrosis, e.g., IPF, COPD, PF-ILD (e.g., SSc), hepatic fibrosis (e.g., liver cirrhosis or chronic hepatic fibrosis)) relative to a control, such as an individual or individuals who are not suffering from the TGFβ-related disorder.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “therapeutically effective amount” refers to an amount of, e.g., an immunotherapeutic agent (such as an immunotherapeutic agent described elsewhere herein) effective to “treat” a disease or disorder in a subject (e.g., a mammal, such as a human).

As used herein, “tocilizumab” is a recombinant humanized monoclonal antibody that binds to human interleukin-6 receptor (IL-6R). It is an IgG1κ (gamma 1, kappa) antibody with a two heavy chains and two light chains forming two antigen-binding sites. In a preferred embodiment, the light chain and heavy chain amino acid sequences of tocilizumab comprise SEQ ID NOs. 187 and 188, respectively.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full-length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs (e.g., CDRs): three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

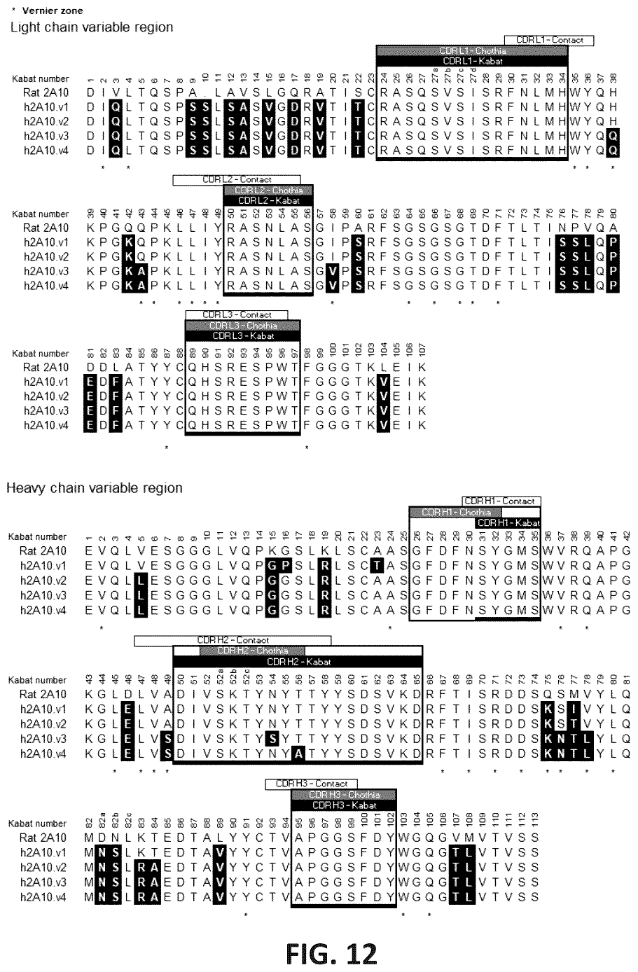

In one embodiment, HVR residues comprise those identified in FIGS. 12-16 and 18-24 or elsewhere in the specification.

Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an isoform-selective anti-TGFβ antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 Daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

Where used herein, “and/or” is to be taken as specific disclosure of each of the two specified features or components with or without the other. For example “A and/or B” is to be taken as specific disclosure of each of (i) A, (ii) B and (iii) A and B, just as if each is set out individually herein.

### II. Compositions and Methods

In one aspect, the invention is based, in part, on the provision of isoform-selective anti-TGFβ antibodies (e.g., monospecific anti-TGFβ2 and anti-TGFβ3 antibodies, and dual-specific, anti-TGFβ2/3 antibodies) and methods of using the same. TGFβ is involved in the control of several key cellular functions including cell proliferation, differentiation, migration, apoptosis and extracellular matrix production. As a consequence, the growth factor influences many biologic processes including embryonic development, wound repair, immune function, malignant transformation and aging. Isoform-selective anti-TGFβ antibodies of the invention are thus useful, e.g., for the diagnosis or treatment of TGFβ-related disorders, such as but not limited to fibrotic disease and cancer.

In some aspects, the epitopes bound by the isoform-selective anti-TGFβ antibodies are provided. The antigen binding domains of the isoform-selective anti-TGFβ antibodies were determined based on their crystal structures. By way of example, the binding epitope of the 2A10 and 4A11 antibodies were mapped by solving their antibody/TGFβ complex crystal structures. See, Example 10, below. As will be appreciated by one of skill in the art, the results from Example 10 demonstrate where anti-TGFβ2/3 antibody 4A11 interacts with TGFβ2 (and, by inference, where it also binds in the same highly conserved region in TGFβ3), and where anti-TGFβ3 antibody 2A10 interacts with TGFβ3. Thus, antibodies that interact with or block any of these residues in TGFβ2 or TGFβ3 can be useful as antibodies that neutralize TGFβ2 or TGFβ3, respectively. In some embodiments, antibodies that, when bound to their target TGFβ isoform(s), interact with or block residues on the TGFβ isoform, or are within 15-8, 8, 8-7, 8-6, 8-5, or 5 angstroms of the residues, are contemplated to provide useful neutralization of the TGFβ isoform(s). By way of non-limiting example, the anti-TGFβ3 antibody 2A10 was determined to bind to an epitope on TGFβ3 containing amino acid residues R325, K331, W332, H334, E335, T387, I388, L389, Y391, V392, G393, R394, P396, K397, and V398 on human TGFβ3 (i.e., the antigen-binding domain directly contacted those residues on TGFβ3), and this binding resulted in neutralization of TGFβ3. Thus, in some embodiments, antibodies that, when bound to TGFβ3, interact with or block those residues on TGFβ3 or are within 15-8, 8, 8-5, or preferably within 5 angstroms of those residues, are contemplated to provide useful neutralization of TGFβ3. By way of further, non-limiting example, the anti-TGFβ2/3 antibody 4A11 was determined to bind to TGFβ2 homodimer, and to directly contact amino acid residues V313, Q314, D315, R320, L322, Y323, R328, D329, F345, and A347 in the first TGFβ2 monomer of the homodimer, and amino acid residues N368, T369, I370, N371, P372, E373, A374, S375, A376, and S377 in the second TGFβ2 monomer. Thus, in some embodiments, antibodies that, when bound to TGFβ2, interact with or block those residues on TGFβ2 or are within 15-8, 8, 8-5, or preferably within 5 angstroms of those residues, are contemplated to provide useful neutralization of TGFβ2. In some embodiments, the antigen binding domain binds within 30, 30-25, 25-20, 20-15, 15-8, 8, 8-5, 5, 5-4, 4 or less angstroms from one or more of the above residues. In some embodiments, the antigen binding domain, when bound to a TGFβ isoform, is within at least one of the above distances, for more than one of the above noted residues. For example, in some embodiments, the antigen binding domain is within one of the recited distances (e.g., 30, 30-25, 25-20, 20-15, 15-8, 8, 8-5, 5, 5-4, 4 or less) for at least 2, 3, 4, 5, 6, 7, 8, 9, 10, 11, 12, 13, 14, 15, 16, 17, 18, 19, 20, 20-25, 25-30, 30-35, 35-40, 40-45, 45-50, 50-55, 55-60, 60-65, 65-70, 70-75 or more of the above residues. In some embodiments, the antigen binding domain is within one of the recited distances for at least 1-10, 10-20, 20-30, 30-40, 40-50, 50-60, 60-70, 70-80, 80-90, 90-95, 95-99, 99-100% of the residues identified in each group of subgroup thereof (such as only those surface residues in the group). Unless specifically stated otherwise, the distance between the antigen binding domain and a TGFβ isoform is the shortest distance between the covalently bonded atom on the TGFβ isoform and the covalently bonded atom of the antigen binding domain that are the closest atoms of the TGFβ isoform and the antigen binding domain. Similarly, unless specifically stated otherwise, the distance between a residue on the antigen-binding domain and the TGFβ isoform for which it is specific is the distance from the closest point on the identified residue to the closest covalently bonded part of the TGFβ isoform, or vice versa. In some embodiments, the distance can be measured from the backbone of the amino acid chains. In some embodiments, the distance can be measured between an edge of the paratope and an edge (closest to one another) of the epitope. In some embodiments, the distance can be measured between the center of the surface of the paratope and the center of the surface of the epitope. As will be appreciated by one of skill in the art, the present description is applicable for each of the individual sets of residues listed herein. For example, the above ranges are contemplated generally and specifically for the epitope and paratope residues listed in Example 10.

### A. Exemplary Isoform-Selective Anti-TGFβ Antibodies

### Anti-TGFβ2 Antibodies

In one aspect, the invention provides isolated antibodies that bind to TGFβ2. In certain embodiments, an anti-TGFβ2 antibody selectively neutralizes TGFβ2. In certain embodiments, an anti-TGFβ2 antibody has one or more of the properties: (a) selectively neutralizes TGFβ2; (b) has reduced toxicity relative to the pan-TGFβ antibody 1D11; (c) has reduced toxicity relative to the pan-TGFβ antibody 1D11; (d) has reduced toxicity in rodents relative to the pan-TGFβ antibody 1D11; (e) has reduced toxicity relative to the pan-TGFβ small molecule inhibitor galunisertib; and/or (f) has reduced toxicity in rodents relative to the pan-TGFβ small molecule inhibitor galunisertib.

In certain aspects, an anti-TGFβ2 antibody provided herein binds to TGFβ2 with a K_{D} of less than 10 pM and/or a cell-based IC₅₀ less than 250 pM. In one aspect, an anti-TGFβ2 antibody provided herein binds to TGFβ2 with a K_{D} of less than or equal to about 5 pM, about 4 pM, about 3 pM, about 2 pM, or about 1 pM. In one aspect, an anti-TGFβ2 antibody provided herein binds to TGFβ2 with a K_{D} of less than 1 pM. In one aspect, an anti-TGFβ2 antibody provided herein has a cell-based IC₅₀ less than or equal to about 250 pM, about 200 pM, about 150 pM, about 100 pM, about 75 pM, or about 50 pM. In one aspect, an anti-TGFβ2 antibody provided herein has a cell-based IC₅₀ for inhibition (neutralization) of TGFβ2 of 40 pM.

In one aspect, the invention provides an anti-TGFβ2 antibody comprising at least one, two, three, four, five, or six HVRs selected from (a) HVR-H1 comprising the amino acid sequence of SEQ ID NO: 16; (b) HVR-H2 comprising the amino acid sequence of SEQ ID NO:17; (c) HVR-H3 comprising the amino acid sequence of SEQ ID NO: 18; (d) HVR-L1 comprising the amino acid sequence of SEQ ID NO: 19; (e) HVR-L2 comprising the amino acid sequence of SEQ ID NO: 20; and (f) HVR-L3 comprising the amino acid sequence of SEQ ID NO: 21.

In one aspect, the invention provides an anti-TGFβ2 antibody comprising at least one, at least two, or all three VH HVR sequences selected from (a) HVR-H1 comprising the amino acid sequence of SEQ ID NO: 16; (b) HVR-H2 comprising the amino acid sequence of SEQ ID NO:17; (c) HVR-H3 comprising the amino acid sequence of SEQ ID NO: 18. In another aspect, the invention provides an antibody comprising (a) HVR-H1 comprising the amino acid sequence of SEQ ID NO: 16; (b) HVR-H2 comprising the amino acid sequence of SEQ ID NO:17; ad (c) HVR-H3 comprising the amino acid sequence of SEQ ID NO: 18.

In another aspect, the invention provides an anti-TGFβ2 antibody comprising at least one, at least two, or all three VL HVR sequences selected from (a) HVR-L1 comprising the amino acid sequence of SEQ ID NO: 19; (b) HVR-L2 comprising the amino acid sequence of SEQ ID NO: 20; and (c) HVR-L3 comprising the amino acid sequence of SEQ ID NO: 21. In one embodiment, the antibody comprises (a) HVR-L1 comprising the amino acid sequence of SEQ ID NO: 19; (b) HVR-L2 comprising the amino acid sequence of SEQ ID NO: 20; and (c) HVR-L3 comprising the amino acid sequence of SEQ ID NO: 21.

In another embodiment, the anti-TGFβ2 antibody comprises HVR-H3 comprising the amino acid sequence of SEQ ID NO: 18 and HVR-L3 comprising the amino acid sequence of SEQ ID NO: 21. In a further embodiment, the anti-TGFβ2 antibody comprises HVR-H3 comprising the amino acid sequence of SEQ ID NO: 18, HVR-L3 comprising the amino acid sequence of SEQ ID NO: 21, and HVR-H2 comprising the amino acid sequence of SEQ ID NO: 17.

In another aspect, an anti-TGFβ2 antibody of the invention comprises (a) a VH domain comprising at least one, at least two, or all three VH HVR sequences selected from (i) HVR-H1 comprising the amino acid sequence of SEQ ID NO: 16; (ii) HVR-H2 comprising the amino acid sequence of SEQ ID NO:17; (iii) HVR-H3 comprising the amino acid sequence of SEQ ID NO: 18; and (b) a VL domain comprising at least one, at least two, or all three VL HVR sequences selected from (i) HVR-L1 comprising the amino acid sequence of SEQ ID NO: 19, (ii) HVR-L2 comprising the amino acid sequence of SEQ ID NO: 20, and (c) HVR-L3 comprising the amino acid sequence of SEQ ID NO: 21.

In another aspect, the invention provides an anti-TGFβ2 antibody comprising a) HVR-H1 comprising the amino acid sequence of SEQ ID NO: 16; (b) HVR-H2 comprising the amino acid sequence of SEQ ID NO:17; (c) HVR-H3 comprising the amino acid sequence of SEQ ID NO: 18; (d) HVR-L1 comprising the amino acid sequence of SEQ ID NO: 19; (e) HVR-L2 comprising the amino acid sequence of SEQ ID NO: 20; and (f) HVR-L3 comprising the amino acid sequence of SEQ ID NO: 21.

In any of the above embodiments, an anti-TGFβ2 antibody is humanized. In one embodiment, an anti-TGFβ2 antibody comprises HVRs as in any of the above embodiments, and further comprises an acceptor human framework, e.g. a human immunoglobulin framework or a human consensus framework. In any of the above embodiments, a humanized anti-TGFβ2 antibody comprises one or more mutations in the VH framework selected from the group consisting of 37V or 37I, 48M or 48L, 49G or 49A, 67L, 71K and 78V, and 105P or 105R. In some embodiments, the anti-TGFβ2 antibody comprises a VH, wherein the VH comprises the amino acid sequence of SEQ ID NO: 25, wherein the VH comprises a set of framework mutations selected from the group consisting of: (i) 37V, 48M and 49G in FR2 and 105P in FR4 (h6F12.v1 and h6F12.v3); (ii) 37V and 48M in FR2, 67L, 71K and 78V in FR3 and 105P in FR4 (h6F12.v2 and h6F12.v4); (iii) 37I in FR2 (h6F12.v1.6); (iv) 48L in FR2 (h6F12.v1.7); (v) 49A in FR2 (h6F12.v1.8); (vi) 105R in FR4 (h6F12.v1.9)(vii) 37V, 48M and 49G in FR2 and 105P in FR4 (h6F12.v1 and h6F12.v3); (viii) 37V and 48M in FR2, 67L, 71K and 78V in FR3 and 105P in FR4 (6F12.v2 and h6F12.v4); (ix) 37I in FR2 (h6F12.v1.6); (x) 48L in FR2 (h6F12.v1.7); (xi): 49A in FR2 (h6F12.v1.8); and (xii) 105R in FR4 (h6F12.v1.9). In any of the above embodiments, a humanized anti-TGFβ2 antibody comprises one or more mutations in the VL framework selected from the group consisting of 43S or 43A, 66G, 69T, 71F, and 87Y. In some embodiments, the anti-TGFβ2 antibody comprises a VL, wherein the VL comprises the amino acid sequence of SEQ ID NO: 24, wherein the VL comprises a set of framework mutations selected from the group consisting of: (i) 43S in FR2 and 66E, 69P, 71Y and 87F in FR3 (h6F12.v1 and h6F12.v2); (ii) 43S in FR2 and 58V, 66E, 69P, 71Y and 87F in FR3 (h6F12.v3 and h6F12.v4); (iii) 43A in FR2 (h6F12.v1.1); (iv) 66G in FR3 (h6F12.v1.2); (v) 69T in FR3 (h6F12.v1.3); (vi) 71F in FR3 (h6F12.v1.4); and (vii) 87Y in FR3 (h6F12.v1.5).

In another aspect, an anti-TGFβ2 antibody comprises a heavy chain variable domain (VH) sequence having at least 90%, 91%, 92%, 93%, 94%, 95%, 96%, 97%, 98%, 99%, or 100% sequence identity to the amino acid sequence selected from the group consisting of SEQ ID NO: 25, 132, and 138-142. In certain embodiments, a VH sequence having at least 90%, 91%, 92%, 93%, 94%, 95%, 96%, 97%, 98%, or 99% sequence identity contains substitutions (e.g., conservative substitutions), insertions, or deletions relative to the reference sequence, but an isoform-selective anti-TGFβ antibody comprising that sequence retains the ability to bind to and selectively neutralize TGFβ2. In certain embodiments, a total of 1 to 10 amino acids have been substituted, inserted and/or deleted in SEQ ID NO: 25. In certain embodiments, substitutions, insertions, or deletions occur in regions outside the HVRs (i.e., in the FRs). Optionally, the anti-TGFβ2 antibody comprises the VH sequence selected from the group consisting of SEQ ID NOs: 25, 132, and 138-142, including post-translational modifications of that sequence. In a particular embodiment, the VH comprises one, two or three HVRs selected from: (a) HVR-H1 comprising the amino acid sequence of SEQ ID NO: 16; (b) HVR-H2 comprising the amino acid sequence of SEQ ID NO: 17; (c) HVR-H3 comprising the amino acid sequence of SEQ ID NO: 18.

In another aspect, an anti-TGFβ2 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence selected from the group consisting of SEQ ID NO: 24, 131, 133-137, 143, and 144. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-TGFβ2 antibody comprising that sequence retains the ability to bind to PRO. In certain embodiments, a total of 1 to 10 amino acids have been substituted, inserted and/or deleted in SEQ ID NO: 24. In certain embodiments, the substitutions, insertions, or deletions occur in regions outside the HVRs (i.e., in the FRs). Optionally, the anti-TGFβ2 antibody comprises the VL sequence selected from the group consisting of SEQ ID NO: 24, 131, 133-137, 143, and 144, including post-translational modifications of that sequence. In a particular embodiment, the VL comprises one, two or three HVRs selected from (a) HVR-L1 comprising the amino acid sequence of SEQ ID NO: 19; (b) HVR-L2 comprising the amino acid sequence of SEQ ID NO: 20; and (c) HVR-L3 comprising the amino acid sequence of SEQ ID NO: 21.

In another aspect, an anti-TGFβ2 antibody is provided, wherein the antibody comprises a VH as in any of the embodiments provided above, and a VL as in any of the embodiments provided above. In one embodiment, the antibody comprises the VH/VL sequences (respectively) selected from the group consisting of SEQ ID NOs: 25/24 (rabbit 6F12), SEQ ID NOs: 132/131 (v1), SEQ ID NOs: 132/133 (v1.1), SEQ ID NOs: 132/134 (v1.2), SEQ ID NOs: 132/135 (v1.3), SEQ ID NOs: 132/136 (v1.4), SEQ ID NOs: 132/137 (v1.5), SEQ ID NOs: 138/131 (v1.6), SEQ ID NOs: 139/131 (v1.7), SEQ ID NOs: 140/131 (v1.8), SEQ ID NOs: 141/131 (v1.9), SEQ ID NOs: 142/131 (v2), SEQ ID NOs: 132/143 (v3), and SEQ ID NOs: 142/144 (v4), including post-translational modifications of those sequences.

In another aspect, an anti-TGFβ2 antibody is provided, wherein the antibody comprises a complete H chain amino acid sequence having at least 95% sequence identity to the amino acid sequence selected from the group consisting of SEQ ID NOs: 31, 146, and 152-156 and/or a complete L chain amino acid sequence having an amino acid sequence selected from the group consisting of SEQ ID NOs: 30, 145, 147-151, 157, and 158. In some embodiments, the anti-TGFβ2 antibody comprises a complete H/L chain pair, the complete H/L chain pair (respectively) comprising amino acid sequences selected from the group consisting of SEQ ID NOs: 31/30 (rabbit 6F12), SEQ ID NOs: 146/145 (v1), SEQ ID NOs: 146/147 (v1.1), SEQ ID NOs: 146/148 (v1.2), SEQ ID NOs: 146/149 (v1.3), SEQ ID NOs: 146/150 (v1.4), SEQ ID NOs: 146/151 (v1.5), SEQ ID NOs: 152/145 (v1.6), SEQ ID NOs: 153/145 (v1.7), SEQ ID NOs: 154/145 (v1.8), SEQ ID NOs: 155/145 (v1.9), SEQ ID NOs: 156/145 (v2), SEQ ID NOs: 146/157 (v3), and SEQ ID NOs: 156/158 (v4).

In a further aspect, the invention provides an antibody that binds to the same epitope as an anti-TGFβ2 antibody provided herein. For example, in certain embodiments, an antibody is provided that binds to the same epitope as an anti-TGFβ2 antibody comprising a) HVR-H1 comprising the amino acid sequence of SEQ ID NO: 16; (b) HVR-H2 comprising the amino acid sequence of SEQ ID NO:17; (c) HVR-H3 comprising the amino acid sequence of SEQ ID NO: 18; (d) HVR-L1 comprising the amino acid sequence of SEQ ID NO: 19; (e) HVR-L2 comprising the amino acid sequence of SEQ ID NO: 20; and (f) HVR-L3 comprising the amino acid sequence of SEQ ID NO: 21.

### Anti-TGFβ2/3 Antibodies

In another aspect, the invention provides isolated antibodies that bind to both TGFβ2 and TGFβ3 (anti-TGFβ2/3 antibodies). In certain aspects, the antibody selectively neutralizes TGFβ2 and TGFβ3, and comprises one or more of the following features:
(a) selectivity of the anti-TGFβ2/3 antibody for TGFβ2 and TGFβ3 over human TGFβ1, with respect to selective neutralization, is achieved by direct contact of the antibody's antigen binding domain with amino acid residue E373 of TGFβ2 or TGFβ3 (human TGFβ2 numbering);
(b) the anti-TGFβ2/3 antibody neutralizes TGFβ2 and/or TGFβ3 via an allosteric mechanism;
(c) the anti-TGFβ2/3 antibody induces a conformational change in TGFβ2 homodimer;
(d) the anti-TGFβ2/3 antibody induces a conformational change in TGFβ2 homodimer, wherein the conformational change comprises the two monomers pinching together by several degrees;
(e) the anti-TGFβ2/3 antibody is a divalent antibody or a monovalent antibody;
(f) the anti-TGFβ2/3 antibody comprises (i) heavy chain CDRs comprising CDR-H1, -H2 and -H3, wherein CDR-H1 has the amino acid sequence of SEQ ID NO: 10, CDR-H2 has the amino acid sequence of SEQ ID NO: 11, and CDR-H3 has the amino acid sequence of SEQ ID NO: 12; and (ii) light chain CDRs comprising CDR-L1, -L2 and -L3, wherein CDR-L1 has the amino acid sequence of SEQ ID NO: 13; CDR-L2 has the amino acid sequence of SEQ ID NO: 14, and CDR-L3 has the amino acid sequence of SEQ ID NO: 15;
(g) the anti-TGFβ2/3 antibody binds to TGFβ2 homodimer, the TGFβ2 homodimer having a first and a second TGFβ2 monomer, and wherein the anti-TGFβ2/3 antibody comprises an antigen binding domain that directly contacts (i) amino acid residues V313, Q314, D315, R320, L322, Y323, R328, D329, F345, and A347 of the first TGFβ2 monomer, and (ii) amino acid residues N368, T369, I370, N371, P372, E373, A374, S375, A376, and S377 of the second TGFβ2 monomer (human TGFβ2 numbering);
(h) the anti-TGFβ2/3 antibody as in (g), wherein the antigen binding domain is within 5 angstroms of the TGFβ2 and/or TGFβ3 amino acid residues;
(i) wherein the anti-TGFβ2/3 antibody binds to a substantially similar epitope as in (g) in TGFβ3; and
(j) the anti-TGFβ2/3 antibody does not neutralize TGFβ2 and/or TGFβ3 in single arm form.

In certain embodiments, an anti-TGFβ2/3 antibody selectively neutralizes TGFβ2 and TGFβ3. In certain aspects, an anti-TGFβ2/3 antibody provided herein binds to TGFβ2/3 with a K_{D} of less than 10 pM and/or a cell-based IC₅₀ less than 250 pM. In one aspect, an anti-TGFβ2/3 antibody provided herein binds to TGFβ2 and/or TGFβ3 with a K_{D} of less than about 10 pM, about 9 pM, about 8 pM, about 7 pM, about 6 pM, about 5 pM, about 4 pM, about 3 pM, about 2 pM, or about 1 pM. In one aspect, an anti-TGFβ2/3 antibody provided herein binds to TGFβ2 with a K_{D} of about 5 pM. In one aspect, an anti-TGFβ2/3 antibody provided herein has a cell-based IC₅₀ for inhibition of TGFβ2 of about 250 pM, about 200 pM, about 150 pM, about 100 pM, about 75 pM, or about 50 pM. In one aspect, an anti-TGFβ2/3 antibody provided herein has a cell-based IC₅₀ for inhibition of TGFβ3 of about 250 pM, about 200 pM, about 150 pM, about 100 pM, about 75 pM, about 50 pM, about 40 pM, about 30 pM or less than about 30 pM. In one aspect, an anti-TGFβ2/3 antibody provided herein has a cell-based IC₅₀ for inhibition of TGFβ2 of about 250 pM and/or a cell-based IC₅₀ for inhibition (neutralization) of TGFβ3 of about 30 pM.

In one aspect, the invention provides an comprising at least one, two, three, four, five, or six HVRs selected from (a) HVR-H1 comprising the amino acid sequence of SEQ ID NO: 10; (b) HVR-H2 comprising the amino acid anti-TGFβ2/3 antibody sequence of SEQ ID NO: 11; (c) HVR-H3 comprising the amino acid sequence of SEQ ID NO: 12; (d) HVR-L1 comprising the amino acid sequence of SEQ ID NO: 13; (e) HVR-L2 comprising the amino acid sequence of SEQ ID NO: 14; and (f) HVR-L3 comprising the amino acid sequence of SEQ ID NO: 15.

In one aspect, the invention provides an anti-TGFβ2/3 antibody comprising at least one, at least two, or all three VH HVR sequences selected from (a) HVR-H1 comprising the amino acid sequence of SEQ ID NO: 10; (b) HVR-H2 comprising the amino acid anti-TGFβ2/3 antibody sequence of SEQ ID NO: 11; (c) HVR-H3 comprising the amino acid sequence of SEQ ID NO: 12. In one embodiment, the antibody comprises HVR-H3 comprising the amino acid sequence of SEQ ID NO: 12. In another embodiment, the antibody comprises HVR-H3 comprising the amino acid sequence of SEQ ID NO: 12 and HVR-L3 comprising the amino acid sequence of SEQ ID NO: 15. In a further embodiment, the antibody comprises HVR-H3 comprising the amino acid sequence of SEQ ID NO: 12, HVR-L3 comprising the amino acid sequence of SEQ ID NO: 15, and HVR-H2 comprising the amino acid sequence of SEQ ID NO: 11. In a further embodiment, the antibody comprises (a) HVR-H1 comprising the amino acid sequence of SEQ ID NO: 10; (b) HVR-H2 comprising the amino acid sequence of SEQ ID NO: 11; and (c) HVR-H3 comprising the amino acid sequence of SEQ ID NO:12.

In another aspect, the invention provides an anti-TGFβ2/3 antibody comprising at least one, at least two, or all three VL HVR sequences selected from (a) HVR-L1 comprising the amino acid sequence of SEQ ID NO: 13; (b) HVR-L2 comprising the amino acid sequence of SEQ ID NO: 14; and (c) HVR-L3 comprising the amino acid sequence of SEQ ID NO: 15. In one embodiment, the anti-TGFβ2/3 antibody comprises (a) HVR-L1 comprising the amino acid sequence of SEQ ID NO: 13; (b) HVR-L2 comprising the amino acid sequence of SEQ ID NO: 14; and (c) HVR-L3 comprising the amino acid sequence of SEQ ID NO: 15.

In another aspect, an anti-TGFβ2/3 antibody of the invention comprises (a) a VH domain comprising at least one, at least two, or all three VH HVR sequences selected from (i) HVR-H1 comprising the amino acid sequence of SEQ ID NO: 10, (ii) HVR-H2 comprising the amino acid sequence of SEQ ID NO: 11, and (iii) HVR-H3 comprising an amino acid sequence selected from SEQ ID NO: 12; and (b) a VL domain comprising at least one, at least two, or all three VL HVR sequences selected from (i) HVR-L1 comprising the amino acid sequence of SEQ ID NO: 13, (ii) HVR-L2 comprising the amino acid sequence of SEQ ID NO: 14, and (c) HVR-L3 comprising the amino acid sequence of SEQ ID NO: 15.

In another aspect, the invention provides an anti-TGFβ2/3 antibody comprising (a) HVR-H1 comprising the amino acid sequence of SEQ ID NO: 10; (b) HVR-H2 comprising the amino acid sequence of SEQ ID NO: 11; (c) HVR-H3 comprising the amino acid sequence of SEQ ID NO: 12; (d) HVR-L1 comprising the amino acid sequence of SEQ ID NO: 13; (e) HVR-L2 comprising the amino acid sequence of SEQ ID NO: 14; and (f) HVR-L3 comprising an amino acid sequence selected from SEQ ID NO: 15.

In any of the above embodiments, an anti-TGFβ2/3 antibody is humanized. In one embodiment, an anti-TGFβ2/3 antibody comprises HVRs as in any of the above embodiments, and further comprises an acceptor human framework, e.g. a human immunoglobulin framework or a human consensus framework. In another embodiment, an anti-TGFβ2/3 antibody comprises HVRs as in any of the above embodiments, and further comprises a VH comprising FR modifications selected from the group consisting of 1E, 2Q or 2V, 24V, 37V or 37I, 48I, 49G, 67F or 67V, 71K or 71V, 73S or 73T, deletion of 75K and 76N, 78V or 78F, 91F or 91Y, 105P or 105Q. In some embodiments, the anti-TGFβ2/3 antibody comprises a VH, wherein the VH comprises the amino acid sequence of SEQ ID NO: 27, wherein the VH comprises a set of framework modifications selected from the group consisting of: (i) 2Q and 24V in FR1, 48I and 49G in FR2, 71K, 73S, 78V and 91F in FR3 and 105P in FR4 (h4A11.v, h4A11.v2, h4A11.v5, h4A11.v6); (ii) 2Q in FR1, 37V in FR2, 67F, 71K, 73S, 78V and 91F in FR3 and 105P in FR4 (h4A11.v3, h4A11.v4, 4A11.v7, h4A11.v8); (iii) delete 1E in FR1 (h4A11.v7.1); (iv) delete 75K and 76N in FR3 (h4A11.v7.2); (v) delete 1E in FR1 and 75K76N in FR3 (h4A11.v7.3); (vi) 2V in FR1 (h4A11.v7.8); (vi) 37I in FR2 (h4A11.v7.9); (vii) 67V in FR3 (h4A11.v7.10); (viii) 71V in FR3 (h4A11.v7.11); (ix) 73T in FR3 (h4A11.v7.12); (x) 78F in FR3 (h4A11.v7.13); (xi) 91Y in FR3 (h4A11.v7.14); (xii) 105Q in FR4 (h4A11.v7.15); (xiii) 2V in FR1, 37I in FR2, 67V, 73T, 78F in FR3, 105Q in FR4 ((h4A11.v7.16); (xiv) 2V in FR1, 37I in FR2, 67V, 73T, 91Y in FR3, 105Q in FR4 (h4A11.v7.17); (xv) 2V in FR1, 37I in FR2, 67V, 73T in FR3, 105Q in FR4 (h4A11.v7.18); and (xvi) 2V in FR1, 37I in FR2, 67V, 73T, deletion of 75K and 76N in FR3, 105Q in FR4 (h4A11.v7.19. In another embodiment, an anti-TGFβ2/3 antibody comprises HVRs as in any of the above embodiments, and further comprises a VL comprising FR modifications selected from the group consisting of: 2A or 2I, 4L, 36F or 36Y, 43P or 43A, and 58V or 58I. In some embodiments, the anti-TGFβ2/3 antibody comprises a VL, wherein the VL comprises the amino acid sequence of SEQ ID NO: 26, wherein the VL comprises a set of framework modifications selected from the group consisting of: (i) 2A and 4L in FR1 and 36F in FR2 (h4A11.v1 and h4A11.v3); (ii) 2A and 4L in FR1 and 36F and 43P in FR2 (h4A11.v2 and h4A11.v4); (iii) 2A in FR1, 36F and 43P in FR2 and 58V in FR3 (h4A11.v5 and h4A11.v7); (iv) 2A and 4L in FR1 and 36F in FR2 (h4A11.v6 and h4A11.v8); (v) 2I in FR1 (h4A11.v7.4); (vi) 36Y in FR2 (h4A11.v7.5); (vii) 43A in FR2 (h4A11.v7.6); (viii) 58I in FR3 (h4A11.v7.7); and (ix) 2I in FR1, 43A in FR2, 58I in FR3 (h4A11.v7.16-19).

In another aspect, an anti-TGFβ2/3 antibody comprises a heavy chain variable domain (VH) sequence having at least 90%, 91%, 92%, 93%, 94%, 95%, 96%, 97%, 98%, 99%, or 100% sequence identity to the amino acid sequence selected from the group consisting of SEQ ID NOs: 27, 81, 83, 86-88, 93-100, and 102-105. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-TGFβ2/3 antibody comprising that sequence retains the ability to bind to TGFβ2 and to TGFβ3. In certain embodiments, a total of 1 to 10 amino acids have been substituted, inserted and/or deleted in SEQ ID NO: 27. In certain embodiments, substitutions, insertions, or deletions occur in regions outside the HVRs (i.e., in the FRs). Optionally, the anti-TGFβ2/3 antibody comprises the VH sequence selected from the group consisting of SEQ ID NO: SEQ ID NOs: 27, 81, 83, 86-88, 93-100, and 102-105, including post-translational modifications of that sequence. In a particular embodiment, the VH comprises one, two or three HVRs selected from: (a) HVR-H1 comprising the amino acid sequence of SEQ ID NO: 10, (b) HVR-H2 comprising the amino acid sequence of SEQ ID NO: 11, and (c) HVR-H3 comprising the amino acid sequence of SEQ ID NO: 12.

In another aspect, an anti-TGFβ2/3 antibody is provided, wherein the anti-TGFβ2/3 antibody comprises a light chain variable domain (VL) having at least 90%, 91%, 92%, 93%, 94%, 95%, 96%, 97%, 98%, 99%, or 100% sequence identity to the amino acid sequence selected from the group consisting of SEQ ID NOs: 26, 80, 82, 84, 85, 89-92, and 101. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-TGFβ2/3 antibody comprising that sequence retains the ability to bind to TGFβ2 and TGFβ3. In certain embodiments, a total of 1 to 10 amino acids have been substituted, inserted and/or deleted in SEQ ID NO: 26. In certain embodiments, the substitutions, insertions, or deletions occur in regions outside the HVRs (i.e., in the FRs).

Optionally, the anti-TGFβ2/3 antibody comprises the VL sequence selected from the group consisting of SEQ ID NOs: 26, 80, 82, 84, 85, 89-92, and 101, including post-translational modifications of that sequence. In a particular embodiment, the VL comprises one, two or three HVRs selected from (a) HVR-L1 comprising the amino acid sequence of SEQ ID NO: 13; (b) HVR-L2 comprising the amino acid sequence of SEQ ID NO: 14; and (c) HVR-L3 comprising the amino acid sequence of SEQ ID NO: 15.

In another aspect, an anti-TGFβ2/3 antibody is provided, wherein the antibody comprises a VH as in any of the embodiments provided above, and a VL as in any of the embodiments provided above. In one embodiment, the anti-TGFβ2/3 antibody comprises the VH and VL sequences (respectively) selected from the group consisting of SEQ ID NOs: 27/26 (rabbit 4A11), SEQ ID NOs: 81/80 (v1), SEQ ID NOs: 81/82 (v2), SEQ ID NOs: 83/80 (v3), SEQ ID NOs: 83/82 (v4), SEQ ID NOs: 81/84 (v5), SEQ ID NOs: 81/85 (v6), SEQ ID NOs: 83/84 (v7), SEQ ID NOs: 86/84 (v7/1), SEQ ID NOs: 87/84 (v7.2), SEQ ID NOs: 88/84 (v7.3), SEQ ID NOs: 83/89 (v7.4), SEQ ID NOs: 83/90 (v7.5), SEQ ID NOs: 83/91 (v7.6), SEQ ID NOs: 83/92 (v7.7), SEQ ID NOs: 93/84 (v7.8), SEQ ID NOs: 94/84 (v7.9), SEQ ID NOs: 95/84 (v7.10), SEQ ID NOs: 96/84 (v7.11), SEQ ID NOs: 97/84 (v7.12), SEQ ID NOs: 98/84 (v7.13), SEQ ID NOs: 99/84 (v7.14), SEQ ID NOs: 100/84 (v7.15), SEQ ID NOs: 102/101 (v7.16), SEQ ID NOs: 103/101 (v7.17), SEQ ID NOs: 104/101 (v7.18), SEQ ID NOs: 105/101 (v7.19), SEQ ID NOs: 83/85 (v8), including post-translational modifications of those sequences.

In another aspect, an anti-TGFβ2/3 antibody is provided, wherein the antibody comprises a complete H chain amino acid sequence having at least 95% sequence identity to the amino acid sequence selected from the group consisting of SEQ ID NOs: 33, 107, 109, 112-114, and 119-130 and/or a complete L chain amino acid sequence having an amino acid sequence selected from the group consisting of SEQ ID NOs: 32, 106, 108, 110, 111, 115-118, and 186. In some embodiments, the anti-TGFβ2/3 antibody comprises a complete H/L chain pair, the complete H/L chain pair (respectively) comprising amino acid sequences selected from the group consisting of SEQ ID NOs: 32/33 (rabbit 4A11), SEQ ID NOs: 107/106 (v1), SEQ ID NOs: 107/108 (v2), SEQ ID NOs: 109/106 (v3), SEQ ID NOs: 109/108 (v4), SEQ ID NOs: 107/110 (v5), SEQ ID NOs: 107/111 (v6), SEQ ID NOs: 109/110 (v7), SEQ ID NOs: 112/110 (v7.1), SEQ ID NOs: 113/110 (v7.2), SEQ ID NOs: 114/110 (v7.3), SEQ ID NOs: 114/115 (v7.4), SEQ ID NOs: 114/116 (v7.5), SEQ ID NOs: 114/117 (v7.6), SEQ ID NOs: 114/118 (v7.7), SEQ ID NOs: 119/110 (v7.8), SEQ ID NOs:120/110 (v7.9), SEQ ID NOs: 121/110 (v7.10), SEQ ID NOs: 122/110 (v7.11), SEQ ID NOs: 123/110 (v7.12), SEQ ID NOs: 124/110 (v7.13), SEQ ID NOs: 125/110 (v7.14), SEQ ID NOs: 126/110 (v7.15), SEQ ID NOs: 127/186 (v7.16), SEQ ID NOs: 128/186 (v7.17), SEQ ID NOs: 129/186 (v7.18), SEQ ID NOs: 130/186 (v7.19), and SEQ ID NOs: 114/111 (v8).

In a further aspect, the invention provides an anti-TGFβ2/3 antibody that binds to the same epitope as an anti-TGFβ2/3 antibody provided herein. For example, in certain embodiments, an antibody is provided that binds to the same epitope as an anti-TGFβ2/3 antibody comprising (a) HVR-H1 comprising the amino acid sequence of SEQ ID NO: 10; (b) HVR-H2 comprising the amino acid sequence of SEQ ID NO: 11; (c) HVR-H3 comprising the amino acid sequence of SEQ ID NO: 12; (d) HVR-L1 comprising the amino acid sequence of SEQ ID NO: 13; (e) HVR-L2 comprising the amino acid sequence of SEQ ID NO: 14; and (f) HVR-L3 comprising an amino acid sequence selected from SEQ ID NO: 15. In certain embodiments, an anti-TGFβ2/3 antibody is provided that binds to an epitope spanning a TGFβ2 homodimer, the TGFβ2 homodimer having a first and a second TGFβ2 monomer, wherein the anti-TGFβ2/3 antibody comprises an antigen-binding domain that directly contacts amino acid residues V313, Q314, D315, R320, L322, Y323, R328, D329, F345, and A347 of the first TGFβ2 monomer, and amino acid residues N368, T369, I370, N371, P372, E373, A374, S375, A376, and S377 of the second TGFβ2 monomer, and wherein, in some embodiments, the anti-TGFβ2/3 antibody binds to the same epitope in TGFβ3. TGFβ2 and TGFβ3 are highly conserved at the region comprising amino acid positions 368-377 (TGFβ2 numbering), thus the anti-TGFβ2/3 antibody binds to same region in both TGFβ2 and TGFβ3.

### Anti-TGFβ3 Antibodies

In one aspect, the invention provides isolated antibodies that bind to TGFβ3. In certain embodiments, an anti-TGFβ3 antibody selectively neutralizes TGFβ3. Further, it is presently discovered that the anti-TGFβ3 antibodies described herein have improved safety profiles (e.g., reduced toxicity) compared to pan-TGFβ inhibitors as well as compared to isoform-selective antibodies specific for TGFβ2 (Example 2) and anti-TGFβ1 selective antibodies. Several inhibitors of TGFβ signaling have been investigated in preclinical toxicology studies, including both small molecule inhibitors of the kinase activity of TGFBR1 (ALK5), e.g., galunisertib, and antibody-based inhibitors of TGFβ-TGFBR interactions, e.g., fresolimumab, a pan-TGFβ antibody. Rat toxicology studies of ALK5 small molecule inhibitors from different chemical series consistently showed hemorrhagic, inflammatory, and degenerative heart valve lesions and physeal dysplasia (Frazier Toxicol Pathol 35, 284-95. 2007; Anderton Toxicol Pathol 39: 916, 2011). In early clinical studies, galunisertib has been dosed at levels that do not completely inhibit ALK5 activity for relatively short periods of time and cardiac findings have not yet emerged in the clinic. Mice treated with pan-TGFβ antibody 1D11 (Lonning et al. (2011) Current Pharmaceutical Biotechnology, 12, 2176-2189) developed histologic lesions, weight loss, nonneoplastic cystic epithelial hyperplasia and inflammation of the tongue and dental dysplasia and epithelial hyperplasia of the gingiva and esophagus. Fresolimumab, a humanized form of GC1008 antibody that binds to and inhibits the activity of all three TGFβ isoforms with comparable affinities for the TGFβ isoforms as 1D11 antibody, was investigated in cynomolgus monkeys and led to dose-dependent bleeding, anemia, and hyperplasia in urinary, nasal, and bladder epithelia. In humans, fresolimumab treatment resulted in anemia and bleeding (gingival, nasal, and subconjunctival), and an increased rate of keratoacanthomas (pre-cancerous squamous skin lesions) that reversed with treatment cessation (Rice, JCI 125:2795 (2015); Lacouture, Cancer Immunol Immunother 64:437 (2015)). CAT-192, an antibody predominantly selective for TGFβ1, had a high serious adverse event rate with multiple gastric hemorrhages observed in a phase 1-2 study in SSc (Denton A&R 56:323 (2007); see, also, the World Wide Web at tripod.nih.gov/ginas/app/substance/4AR67180L0). Taken together, these observations suggest that bleeding, cardiac lesions, and epithelial hyperplasia are significant concerns with long-term chronic pharmacologic TGFβ inhibition.

In contrast to the concerning safety issues that have been observed with past attempts to neutralize TGFβ in vivo, in certain aspects, the present invention provides anti-TGFβ3 antibodies having improved safety profiles (e.g., reduced toxicity). For example, in Example 2, below, mice treated with an anti-TGFβ3 antibody at doses up to 50 mg/kg administered thrice weekly for a total of 4-weeks had no or very minor side effects (physeal dysplasia at the highest dose), and none of the serious side effects caused by small molecule inhibitors, anti-TGFβ1 or pan-TGFβ antibodies, as discussed above, were observed. The anti-TGFβ3 antibodies described herein were also discovered to have improved safety profiles relative to isoform-selective anti-TGFβ2/3 and anti-TGFβ2 antibodies (Example 2, see Table 8).

In certain aspects, an isolated anti-TGFβ3 antibody is provided, wherein the antibody selectively neutralizes TGFβ3, and wherein the antibody comprises one or more of the following features:
(a) the anti-TGFβ3 antibody binds to the beta6/beta7 hairpin region of TGFβ3;
(b) binding of the anti-TGFβ3 antibody sterically blocks the ability of TGFBR2, but not TGFBR1, to bind TGFβ3;
(c) binding of the anti-TGFβ3 antibody to TGFβ3 blocks TGFBR2 binding and inhibits the TGFBR1/TGFBR2 signaling receptors from binding to TGFβ3;
(d) the anti-TGFβ3 antibody binds amino acid residue R394 in the beta6/beta7 hairpin region of human TGFβ3;
(e) the anti-TGFβ3 antibody binds amino acid residue R394 in the beta6/beta7 hairpin region of human TGFβ3 and residue R394 of TGFβ3 makes an ionic salt bridge with the anti-TGFβ3 antibody in the heavy chain CDR2;
(f) isoform selectivity of the anti-TGFβ3 antibody for TGFβ3 over TGFβ1 is achieved by direct contact by the antigen binding domain of the anti-TGFβ3 antibody with amino acid residues T387, L389, and T395 of TGFβ3 (human TGFβ3 numbering);
(g) the isoform selectivity of the anti-TGFβ3 antibody for TGFβ3 over TGFβ2 is achieved by direct contact by the antigen binding domain of the anti-TGFβ3 antibody with amino acid residues R325, R394, and V398 of TGFβ3 (human TGFβ3 numbering);
(h) the anti-TGFβ3 antibody has reduced toxicity relative to the pan-TGFβ antibody 1D11;
(i) the anti-TGFβ3 antibody has reduced toxicity in rodents or cynomolgus monkeys relative to the pan-TGFβ antibody 1D11;
(j) the anti-TGFβ3 antibody has reduced toxicity relative to the pan-TGFβ small molecule inhibitor galunisertib;
(k) the anti-TGFβ3 antibody has reduced toxicity in rodents relative to the pan-TGFβ small molecule inhibitor galunisertib;
(l) the anti-TGFβ3 antibody has reduced toxicity relative to the anti-TGFβ1 antibody CAT-192;
(m) the anti-TGFβ3 antibody has reduced toxicity relative to an isoform selective anti-TGFβ2 antibody and/or anti-TGFβ2/3 antibody.
(n) the anti-TGFβ3 antibody comprises: (i) heavy chain CDRs comprising CDR-H1, -H2 and -H3, wherein CDR-H1 has the amino acid sequence of SEQ ID NO: 4, CDR-H2 has the amino acid sequence of one of SEQ ID NOs: 5, 34, 35, and 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9;
(o) the anti-TGFβ3 antibody comprises an antigen binding domain that directly contacts amino acid residues R325, K331, W332, H334, E335, T387, I388, L389, Y391, V392, G393, R394, P396, K397, and V398 on human TGFβ3; and
(p) the anti-TGFβ3 antibody as in (o), wherein the antigen binding domain is within 15-8, 8, 8-5, 7-5, 6-5, or 5 angstroms of the TGFβ3 amino acid residues.

In certain aspects, an anti-TGFβ3 antibody provided herein binds to TGFβ3 with a K_{D} of less than 10 pM and/or a cell-based IC₅₀ less than 250 pM. In one aspect, an anti-TGFβ3 antibody provided herein binds to TGFβ3 with a K_{D} of less than about 5 pM, about 4 pM, about 3 pM. In one aspect, an anti-TGFβ3 antibody provided herein binds to TGFβ3 with a K_{D} of less than about 2 pM. In one aspect, an anti-TGFβ3 antibody provided herein has a cell-based IC₅₀ less than about 200 pM, about 150 pM, about 100 pM, about 75 pM, or about 50 pM. In one aspect, an anti-TGFβ3 antibody provided herein has a cell-based IC₅₀ for inhibition (neutralization) of TGFβ3 of less than about 20 pM.

In another aspect, an anti-TGFβ3 antibody provided herein selectively neutralizes TGFβ3 and has reduced toxicity in mice relative to the pan-TGFβ antibody 1D11 (see, Lonning et al. (2011)). In another aspect, an anti-TGFβ3 antibody provided herein selectively neutralizes TGFβ3 and has an improved safety profile, e.g., relative to the pan-TGFβ inhibitors such as the ALK5 inhibitors described in Anderton et al. and/or 1D11 antibody described in Lonning et al. (2011). In another aspect, an anti-TGFβ3 antibody provided herein selectively neutralizes TGFβ3 and has reduced toxicity in mice relative to the anti-TGFβ1 antibody CAT-192/metelimumab.

In certain aspects, the CAT-192 antibody has the following heavy and light chain variable amino acid sequences:

VH
(SEQ ID NO: 184)
EVQLVESGGGVVQPGRSLRLSCAASGFTFSSYGMHWVRQAPGKELEWVA
VISYDGSIKYYADSVKGRFTISRDNSKNTLYLQMNSLRAEDTAVYYCAR
TGEYSGYDTDPQYSWGQGTTVTVSSASTKGPSVFPLAPCSRSTSESTAA
LGCLVKDYFPEPVTVSWNSGALTSGVHTFPAVLQSSGLYSLSSVVTVPS
SSLGTKTYTCNVDHKPSNTKVDKRVESKYGPPCPSCPAPEFLGGPSVFL
FPPKPKPTLMISRTPEVTCVVVDVSQEDPEVQFNWYVDGVEVHNAKTKP
REEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPEN
NYKTTPPVLDSDGSFFLYSRLTVDKSRWQEGNVFSCSVMHEALHNHYTQ
KSLSLSLGK.
VL
(SEQ ID NO: 185)
EIVLTQSPSSLSASVGDRVTITCRASQGIGDDLGWYQQKPGKAPILLIY
GTSTLQSGVPSRFSGSGSGTDFTLTINSLQPEDFATYYCLQDSNYPLTF
GGGTRLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSPVTKSFNRGEC.

In any of the above aspects, the anti-TGFβ3 antibody comprising at least one, two, three, four, five, or six HVRs selected from (a) HVR-H1 comprising the amino acid sequence of SEQ ID NO: 4; (b) HVR-H2 comprising the amino acid sequence selected from SEQ ID NOs: 5, 34, 35, and 159; (c) HVR-H3 comprising the amino acid sequence of SEQ ID NO: 6; (d) HVR-L1 comprising the amino acid sequence of SEQ ID NO: 7; (e) HVR-L2 comprising the amino acid sequence of SEQ ID NO: 8; and (f) HVR-L3 comprising the amino acid sequence of SEQ ID NO: 9.

In one aspect, the invention provides an anti-TGFβ3 antibody comprising at least one, at least two, or all three VH HVR sequences selected from (a) HVR-H1 comprising the amino acid sequence of SEQ ID NO: 4; (b) HVR-H2 comprising the amino acid sequence of SEQ ID NO: 5; and (c) HVR-H3 comprising the amino acid sequence of SEQ ID NO: 6. In another aspect, the invention provides an anti-TGFβ3 antibody comprising at least one, at least two, or all three VH HVR sequences selected from (a) HVR-H1 comprising the amino acid sequence of SEQ ID NO: 4; (b) HVR-H2 comprising the amino acid sequence of SEQ ID NO: 34; and (c) HVR-H3 comprising the amino acid sequence of SEQ ID NO: 6. In another aspect, the invention provides an anti-TGFβ3 antibody comprising at least one, at least two, or all three VH HVR sequences selected from (a) HVR-H1 comprising the amino acid sequence of SEQ ID NO: 4; (b) HVR-H2 comprising the amino acid sequence of SEQ ID NO: 35; and (c) HVR-H3 comprising the amino acid sequence of SEQ ID NO: 6. In another aspect, the invention provides an anti-TGFβ3 antibody comprising at least one, at least two, or all three VH HVR sequences selected from (a) HVR-H1 comprising the amino acid sequence of SEQ ID NO: 4; (b) HVR-H2 comprising the amino acid sequence of SEQ ID NO: 159; and (c) HVR-H3 comprising the amino acid sequence of SEQ ID NO: 6.

In one embodiment, the antibody comprises HVR-H3 comprising the amino acid sequence of SEQ ID NO: 6. In another embodiment, the antibody comprises HVR-H3 comprising the amino acid sequence of SEQ ID NO: 6 and HVR-L3 comprising the amino acid sequence of SEQ ID NO: 9. In a further embodiment, the antibody comprises HVR-H3 comprising the amino acid sequence of SEQ ID NO: 6, HVR-L3 comprising the amino acid sequence of SEQ ID NO: 9, and HVR-H2 comprising the amino acid sequence of SEQ ID NO: 5. In a further embodiment, the anti-TGFβ3 antibody comprises HVR-H3 comprising the amino acid sequence of SEQ ID NO: 6, HVR-L3 comprising the amino acid sequence of SEQ ID NO: 9, and HVR-H2 comprising the amino acid sequence of SEQ ID NO: 34. In a further embodiment, the anti-TGFβ3 antibody comprises HVR-H3 comprising the amino acid sequence of SEQ ID NO: 6, HVR-L3 comprising the amino acid sequence of SEQ ID NO: 9, and HVR-H2 comprising the amino acid sequence of SEQ ID NO: 35. In a further embodiment, the anti-TGFβ3 antibody comprises HVR-H3 comprising the amino acid sequence of SEQ ID NO: 6, HVR-L3 comprising the amino acid sequence of SEQ ID NO: 9, and HVR-H2 comprising the amino acid sequence of SEQ ID NO: 159. In a further embodiment, the anti-TGFβ3 antibody comprises (a) HVR-H1 comprising the amino acid sequence of SEQ ID NO: 4; (b) HVR-H2 comprising the amino acid sequence of SEQ ID NO: 5; and (c) HVR-H3 comprising the amino acid sequence of SEQ ID NO:6. In a further embodiment, the anti-TGFβ3 antibody comprises (a) HVR-H1 comprising the amino acid sequence of SEQ ID NO: 4; (b) HVR-H2 comprising the amino acid sequence of SEQ ID NO: 34; and (c) HVR-H3 comprising the amino acid sequence of SEQ ID NO:6. In a further embodiment, the anti-TGFβ3 antibody comprises (a) HVR-H1 comprising the amino acid sequence of SEQ ID NO: 4; (b) HVR-H2 comprising the amino acid sequence of SEQ ID NO: 35; and (c) HVR-H3 comprising the amino acid sequence of SEQ ID NO:6. In a further embodiment, the anti-TGFβ3 antibody comprises (a) HVR-H1 comprising the amino acid sequence of SEQ ID NO: 4; (b) HVR-H2 comprising the amino acid sequence of SEQ ID NO: 159; and (c) HVR-H3 comprising the amino acid sequence of SEQ ID NO:6.

In another aspect, the invention provides an anti-TGFβ3 antibody comprising at least one, at least two, or all three VL HVR sequences selected from (a) HVR-L1 comprising the amino acid sequence of SEQ ID NO: 7; (b) HVR-L2 comprising the amino acid sequence of SEQ ID NO: 8; and (c) HVR-L3 comprising the amino acid sequence of SEQ ID NO: 9. In one embodiment, the antibody comprises (a) HVR-L1 comprising the amino acid sequence of SEQ ID NO: 7; (b) HVR-L2 comprising the amino acid sequence of SEQ ID NO: 8; and (c) HVR-L3 comprising the amino acid sequence of SEQ ID NO: 9.

In another aspect, an anti-TGFβ3 antibody of the invention comprises (a) a VH domain comprising at least one, at least two, or all three VH HVR sequences selected from (i) HVR-H1 comprising the amino acid sequence of SEQ ID NO: 4, (ii) HVR-H2 comprising the amino acid sequence of SEQ ID NO: 5, and (iii) HVR-H3 comprising an amino acid sequence selected from SEQ ID NO: 6; and (b) a VL domain comprising at least one, at least two, or all three VL HVR sequences selected from (i) HVR-L1 comprising the amino acid sequence of SEQ ID NO: 7, (ii) HVR-L2 comprising the amino acid sequence of SEQ ID NO: 8, and (c) HVR-L3 comprising the amino acid sequence of SEQ ID NO: 9. In another aspect, an anti-TGFβ3 antibody of the invention comprises (a) a VH domain comprising at least one, at least two, or all three VH HVR sequences selected from (i) HVR-H1 comprising the amino acid sequence of SEQ ID NO: 4, (ii) HVR-H2 comprising the amino acid sequence of SEQ ID NO: 34, and (iii) HVR-H3 comprising an amino acid sequence selected from SEQ ID NO: 6; and (b) a VL domain comprising at least one, at least two, or all three VL HVR sequences selected from (i) HVR-L1 comprising the amino acid sequence of SEQ ID NO: 7, (ii) HVR-L2 comprising the amino acid sequence of SEQ ID NO: 8, and (c) HVR-L3 comprising the amino acid sequence of SEQ ID NO: 9. In another aspect, an anti-TGFβ3 antibody of the invention comprises (a) a VH domain comprising at least one, at least two, or all three VH HVR sequences selected from (i) HVR-H1 comprising the amino acid sequence of SEQ ID NO: 4, (ii) HVR-H2 comprising the amino acid sequence of SEQ ID NO: 35, and (iii) HVR-H3 comprising an amino acid sequence selected from SEQ ID NO: 6; and (b) a VL domain comprising at least one, at least two, or all three VL HVR sequences selected from (i) HVR-L1 comprising the amino acid sequence of SEQ ID NO: 7, (ii) HVR-L2 comprising the amino acid sequence of SEQ ID NO: 8, and (c) HVR-L3 comprising the amino acid sequence of SEQ ID NO: 9. In another aspect, an anti-TGFβ3 antibody of the invention comprises (a) a VH domain comprising at least one, at least two, or all three VH HVR sequences selected from (i) HVR-H1 comprising the amino acid sequence of SEQ ID NO: 4, (ii) HVR-H2 comprising the amino acid sequence of SEQ ID NO: 159, and (iii) HVR-H3 comprising an amino acid sequence selected from SEQ ID NO: 6; and (b) a VL domain comprising at least one, at least two, or all three VL HVR sequences selected from (i) HVR-L1 comprising the amino acid sequence of SEQ ID NO: 7, (ii) HVR-L2 comprising the amino acid sequence of SEQ ID NO: 8, and (c) HVR-L3 comprising the amino acid sequence of SEQ ID NO: 9.

In another aspect, the invention provides an anti-TGFβ3 antibody comprising (a) HVR-H1 comprising the amino acid sequence of SEQ ID NO: 4; (b) HVR-H2 comprising the amino acid sequence of SEQ ID NO: 5; (c) HVR-H3 comprising the amino acid sequence of SEQ ID NO: 6; (d) HVR-L1 comprising the amino acid sequence of SEQ ID NO: 7; (e) HVR-L2 comprising the amino acid sequence of SEQ ID NO: 8; and (f) HVR-L3 comprising an amino acid sequence selected from SEQ ID NO: 9. In another aspect, the invention provides an anti-TGFβ3 antibody comprising (a) HVR-H1 comprising the amino acid sequence of SEQ ID NO: 4; (b) HVR-H2 comprising the amino acid sequence of SEQ ID NO: 34; (c) HVR-H3 comprising the amino acid sequence of SEQ ID NO: 6; (d) HVR-L1 comprising the amino acid sequence of SEQ ID NO: 7; (e) HVR-L2 comprising the amino acid sequence of SEQ ID NO: 8; and (f) HVR-L3 comprising an amino acid sequence selected from SEQ ID NO: 9. In another aspect, the invention provides an anti-TGFβ3 antibody comprising (a) HVR-H1 comprising the amino acid sequence of SEQ ID NO: 4; (b) HVR-H2 comprising the amino acid sequence of SEQ ID NO: 35; (c) HVR-H3 comprising the amino acid sequence of SEQ ID NO: 6; (d) HVR-L1 comprising the amino acid sequence of SEQ ID NO: 7; (e) HVR-L2 comprising the amino acid sequence of SEQ ID NO: 8; and (f) HVR-L3 comprising an amino acid sequence selected from SEQ ID NO: 9. In another aspect, the invention provides an anti-TGFβ3 antibody comprising (a) HVR-H1 comprising the amino acid sequence of SEQ ID NO: 4; (b) HVR-H2 comprising the amino acid sequence of SEQ ID NO: 159; (c) HVR-H3 comprising the amino acid sequence of SEQ ID NO: 6; (d) HVR-L1 comprising the amino acid sequence of SEQ ID NO: 7; (e) HVR-L2 comprising the amino acid sequence of SEQ ID NO: 8; and (f) HVR-L3 comprising an amino acid sequence selected from SEQ ID NO: 9.

In certain embodiments, any one or more amino acids of an anti-TGFβ3 antibody as provided above are substituted at the following HVR positions:
in HVR-H2 (SEQ ID NO: 5): at position N54 (e.g., N54S, N54Q) or T56 (e.g., T56A). In certain embodiments, the substitutions are conservative substitutions, as provided herein.

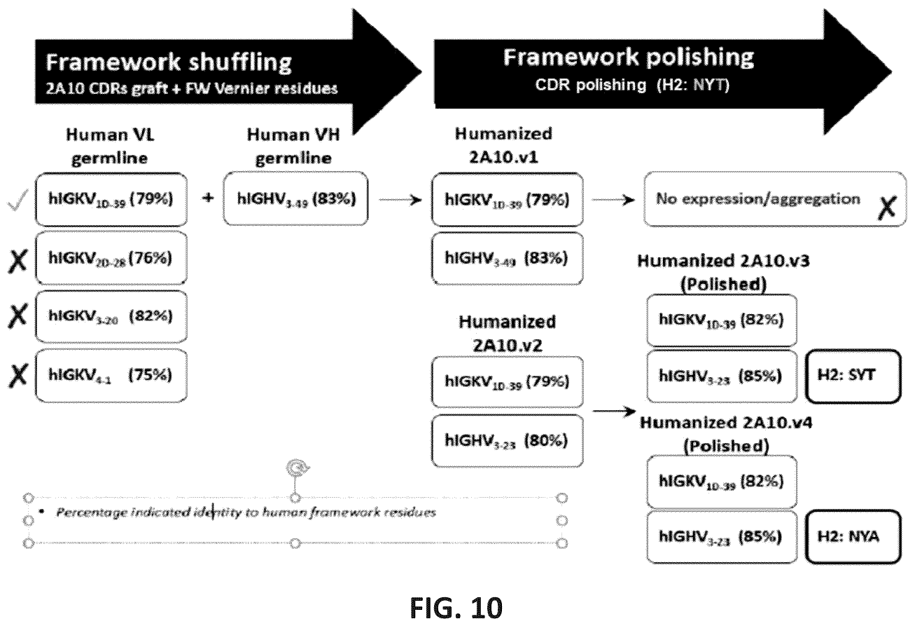

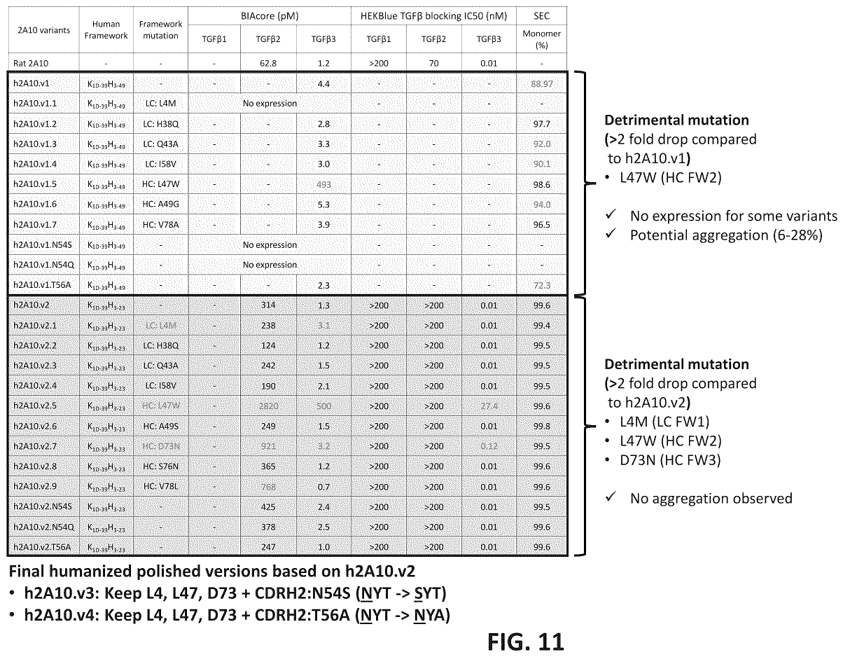

In any of the above embodiments, an anti-TGFβ3 antibody is humanized. In some aspects, an anti-TGFβ3 antibody provided herein that has undergone one or more humanization steps has TGFβ blocking ability similar to the parent antibody, and/or human TGFβ binding similar to the parent antibody, and/or maintains solubility, and/or is able to be expressed, whereas such capabilities may be unknown or not observed in variants potentially or actually derived from other TGFβ3 antibodies (Example 6, Tables 9 and 10; FIGS. 10, 11). In one embodiment, an anti-TGFβ3 antibody comprises HVRs as in any of the above embodiments, and further comprises an acceptor human framework, e.g. a human immunoglobulin framework or a human consensus framework. In another embodiment, an anti-TGFβ3 antibody comprises HVRs as in any of the above embodiments, and further comprises a VH comprising FR modifications selected from the group consisting of 47L or 47W; 49A, 49S, or 49G; 73D or 73N; and 76N, 78D or 78L, 78A, or 78V. In some embodiments, the anti-TGFβ3 antibody comprises a VH, wherein the VH comprises the amino acid sequence of SEQ ID NO: 23, wherein the VH comprises a set of framework modifications selected from the group consisting of: (i) 47L, 49A in FR2, 78V in FR3 (h2A10.v1); (ii) 47L, 49A in FR2, 73D, 76S, 78V in FR3 (h2A10.v2); (iii) 47W in FR2 (h2A10.v1.5); (iv): 49G in FR2 (h2A10.v1.6); (v) 78A in FR3 (h2A10.v1.7); (vi) 47W in FR2 (h2A10.v2.5); (vii) 49S in FR2 (h2A10.v2.6); (viii) 73N in FR3 (h2A10.v2.7); (ix) 76N in FR3 (h2A10.v2.8); (x) 78L in FR3 (h2A10.v2.9); and (xi) 49S in FR2, 76N, 78L in FR3 (h2A10.v3 and h2A10.v4). In another embodiment, an anti-TGFβ3 antibody comprises HVRs as in any of the above embodiments, and further comprises a VL comprising FR modifications selected from the group consisting of: 4L or 4M, 38H or 38Q, 43A or 43Q, and 58V (relative to the VL amino acid sequence of SEQ ID NO: 22). In another embodiment, the antibody comprises the VL of SEQ ID NO: 22, wherein the VL comprises a set of framework modifications selected from the group consisting of: (i) 4L in FR1, 38H and 43Q in FR2, 58I in FR3 (h2A10.v1 and h2A10.v2); (ii) 4M in FR1 (h2A10.v1.1 and h2A10.v2.1); (iii) 38Q in FR2 (h2A10.v1.2 and h2A10.v2.2); (iv) 43A in FR2 (h2A10.v1.3 and h2A10.v2.3); (v) 58V in FR3 (h2A10.v1.4 and h2A10.v2.4); (vi) 38Q, 43A in FR2, 58V in FR3 (h2A10.v3 and h2A10.v4); (vii) 58V in FR3 (h2A10.v1.4 and h2A10.v2.4); and (vi) 38Q, 43A in FR2, 58V in FR3 (h2A10.v3 and h2A10.v4).

In another aspect, an anti-TGFβ3 antibody comprises a heavy chain variable domain (VH) sequence having at least 90%, 91%, 92%, 93%, 94%, 95%, 96%, 97%, 98%, 99%, or 100% sequence identity to the amino acid sequence selected from the group consisting of SEQ ID NOs: 23, 37, 42-50, 55, and 57. In another aspect, an anti-TGFβ3 antibody comprises a heavy chain variable domain (VH) sequence having at least 90%, 91%, 92%, 93%, 94%, 95%, 96%, 97%, 98%, 99%, or 100% sequence identity to the amino acid sequence selected from the group consisting of SEQ ID NOs: 23, 37, and 42-50. In some aspects, an anti-TGFβ3 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 57. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-TGFβ3 antibody comprising that sequence retains the ability to bind to TGFβ3. In certain embodiments, a total of 1 to 10 amino acids have been substituted, inserted and/or deleted in SEQ ID NO: 23. In certain embodiments, substitutions, insertions, or deletions occur in regions outside the HVRs (i.e., in the FRs). Optionally, the anti-TGFβ3 antibody comprises the VH sequence selected from the group consisting of SEQ ID NOs: 23, 37, 42-50, 55, and 57, including post-translational modifications of that sequence. Optionally, the anti-TGFβ3 antibody comprises the VH sequence selected from the group consisting of SEQ ID NOs: 23, 37, and 42-50, including post-translational modifications of that sequence. In a particular embodiment, the VH comprises one, two or three HVRs selected from: (a) HVR-H1 comprising the amino acid sequence of SEQ ID NO: 4, (b) HVR-H2 comprising the amino acid sequence of SEQ ID NO: 5, and (c) HVR-H3 comprising the amino acid sequence of SEQ ID NO: 6. In a particular embodiment, the VH comprises one, two or three HVRs selected from: (a) HVR-H1 comprising the amino acid sequence of SEQ ID NO: 4, (b) HVR-H2 comprising the amino acid sequence of SEQ ID NO: 34, and (c) HVR-H3 comprising the amino acid sequence of SEQ ID NO: 6. In a particular embodiment, the VH comprises one, two or three HVRs selected from: (a) HVR-H1 comprising the amino acid sequence of SEQ ID NO: 4, (b) HVR-H2 comprising the amino acid sequence of SEQ ID NO: 35, and (c) HVR-H3 comprising the amino acid sequence of SEQ ID NO: 6. In a particular embodiment, the VH comprises one, two or three HVRs selected from: (a) HVR-H1 comprising the amino acid sequence of SEQ ID NO: 4, (b) HVR-H2 comprising the amino acid sequence of SEQ ID NO: 159, and (c) HVR-H3 comprising the amino acid sequence of SEQ ID NO: 6.

In another aspect, an anti-TGFβ3 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence selected from the group consisting of SEQ ID NOs: 22, 36, 38-41, 54, and 56. In another aspect, an anti-TGFβ3 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence selected from the group consisting of SEQ ID NOs: 22, 36, and 38-41. In some aspects, an anti-TGFβ3 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence SEQ ID NO: 56. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-TGFβ3 antibody comprising that sequence retains the ability to bind to TGFβ3. In certain embodiments, a total of 1 to 10 amino acids have been substituted, inserted and/or deleted in SEQ ID NO: 22. In certain embodiments, the substitutions, insertions, or deletions occur in regions outside the HVRs (i.e., in the FRs). Optionally, the anti-TGFβ3 antibody comprises the VL sequence selected from the group consisting of SEQ ID NOs: 22, 36, 38-41, 54, and 56, including post-translational modifications of that sequence. Optionally, the anti-TGFβ3 antibody comprises the VL sequence selected from the group consisting of SEQ ID NOs: 22, 36, and 38-41, including post-translational modifications of that sequence. In a particular embodiment, the VL comprises one, two or three HVRs selected from (a) HVR-L1 comprising the amino acid sequence of SEQ ID NO: 7; (b) HVR-L2 comprising the amino acid sequence of SEQ ID NO: 8; and (c) HVR-L3 comprising the amino acid sequence of SEQ ID NO: 9.

In another aspect, an anti-TGFβ3 antibody is provided, wherein the antibody comprises a VH as in any of the embodiments provided above, and a VL as in any of the embodiments provided above. In one embodiment, the anti-TGFβ3 antibody comprises VH/VL sequences (respectively) selected from the group consisting of SEQ ID NOs: 23/22 (rat 2A10), SEQ ID NOs: 37/36 (v1), SEQ ID NOs: 37/38 (v1.1), SEQ ID NOs: 37/39 (v1.2), SEQ ID NOs: 37/40 (v1.3), SEQ ID NOs: 37/41 (v1.4), SEQ ID NOs: 42/36 (v1.5), SEQ ID NOs: 43/36 (v1.6), SEQ ID NOs:44/36 (v1.7), SEQ ID NOs: 45/36 (v2), SEQ ID NOs: 45/38 (v2.1), SEQ ID NOs: 45/39 (v2.2), SEQ ID NOs: 45/40 (v2.3), SEQ ID NOs: 45/41 (v2.4), SEQ ID NOs: 46/36 (v2.5), SEQ ID NOs: 47/36 (v2.6), SEQ ID NOs: 48/36 (v2.7), SEQ ID NOs: 49/36 (v2.8), SEQ ID NOs: 50/36 (v2.9), SEQ ID NOs: 55/54 (v3), and SEQ ID NOs: 57/56 (v4), including post-translational modifications of those sequences. In one embodiment, the anti-TGFβ3 antibody comprises VH/VL sequences (respectively) selected from the group consisting of SEQ ID NOs: 23/22 (rat 2A10), SEQ ID NOs: 37/36 (v1), SEQ ID NOs: 37/38 (v1.1), SEQ ID NOs: 37/39 (v1.2), SEQ ID NOs: 37/40 (v1.3), SEQ ID NOs: 37/41 (v1.4), SEQ ID NOs: 42/36 (v1.5), SEQ ID NOs: 43/36 (v1.6), SEQ ID NOs:44/36 (v1.7), SEQ ID NOs: 45/36 (v2), SEQ ID NOs: 45/38 (v2.1), SEQ ID NOs: 45/39 (v2.2), SEQ ID NOs: 45/40 (v2.3), SEQ ID NOs: 45/41 (v2.4), SEQ ID NOs: 46/36 (v2.5), SEQ ID NOs: 47/36 (v2.6), SEQ ID NOs: 48/36 (v2.7), SEQ ID NOs: 49/36 (v2.8), and SEQ ID NOs: 50/36 (v2.9), including post-translational modifications of those sequences. In some embodiments, the anti-TGFβ3 antibody comprises VH/VL sequences (respectively) of SEQ ID NOs: 57/56.

In another aspect, an anti-TGFβ3 antibody is provided, wherein the antibody comprises a complete H chain amino acid sequence having at least 95% sequence identity to the amino acid sequence selected from the group consisting of SEQ ID NOs: 29, 59, 64, 65-72, 77, and 79 and/or a complete L chain amino acid sequence having an amino acid sequence selected from the group consisting of SEQ ID NOs: 28, 58, 60-63, 76, and 78. In another aspect, an anti-TGFβ3 antibody is provided, wherein the antibody comprises a complete H chain amino acid sequence having at least 95% sequence identity to the amino acid sequence selected from the group consisting of SEQ ID NOs: 29, 59, 64, and 65-72 and/or a complete L chain amino acid sequence having an amino acid sequence selected from the group consisting of SEQ ID NOs: 28, 58, and 60-63. In some embodiments, the antibody comprises a complete H chain amino acid sequence having at least 95% sequence identity to SEQ ID NO: 79, and/or a complete L chain amino acid sequence of SEQ ID NO: 78. In some embodiments, the anti-TGFβ3 antibody comprises a complete H/L chain pair, the complete H/L chain pair (respectively) comprising amino acid sequences selected from the group consisting of SEQ ID NOs: 29/28 (rat 2A10), SEQ ID NOs: 59/58 (v1), SEQ ID NOs: 59/60 (v1.1), SEQ ID NOs: 59/61 (v1.2), SEQ ID NOs: 59/62 (v1.3), SEQ ID NOs: 59/63 (v1.4), SEQ ID NOs: 64/58 (v1.5), SEQ ID NOs: 65/58 (v1.6), SEQ ID NOs: 66/58 (v1.7), SEQ ID NOs: 67/58 (v2), SEQ ID NOs: 67/60 (v2.1), SEQ ID NOs: 67/61 (v2.2), SEQ ID NOs: 67/62 (v2.3), SEQ ID NOs: 67/63 (v2.4), SEQ ID NOs: 68/58 (v2.5), SEQ ID NOs: 69/58 (v2.6), SEQ ID NOs: 70/58 (v2.7), SEQ ID NOs: 71/58 (v2.8), SEQ ID NOs: 72/58 (v2.9), SEQ ID NOs: 77/76 (v3), and SEQ ID NOs: 79/78 (v4). In some embodiments, the anti-TGFβ3 antibody comprises a complete H/L chain pair, the complete H/L chain pair (respectively) comprising amino acid sequences selected from the group consisting of SEQ ID NOs: 29/28 (rat 2A10), SEQ ID NOs: 59/58 (vi), SEQ ID NOs: 59/60 (v1.1), SEQ ID NOs: 59/61 (v1.2), SEQ ID NOs: 59/62 (v1.3), SEQ ID NOs: 59/63 (v1.4), SEQ ID NOs: 64/58 (v1.5), SEQ ID NOs: 65/58 (v1.6), SEQ ID NOs: 66/58 (v1.7), SEQ ID NOs: 67/58 (v2), SEQ ID NOs: 67/60 (v2.1), SEQ ID NOs: 67/61 (v2.2), SEQ ID NOs: 67/62 (v2.3), SEQ ID NOs: 67/63 (v2.4), SEQ ID NOs: 68/58 (v2.5), SEQ ID NOs: 69/58 (v2.6), SEQ ID NOs: 70/58 (v2.7), SEQ ID NOs: 71/58 (v2.8), SEQ ID NOs: 72/58 (v2.9).

In another embodiment, an anti-TGFβ3 antibody is provided, wherein the antibody comprises a VH having at least 90%, 91%, 92%, 93%, 94%, 95%, 96%, 97%, 98%, 99%, or 100% sequence identity to the amino acid sequence of SEQ ID NO: 52. In some aspects, the anti-TGFβ3 antibody comprises a VL amino acid sequence having at least 90%, 91%, 92%, 93%, 94%, 95%, 96%, 97%, 98%, 99%, or 100% sequence identity to an amino acid sequence selected from the group consisting of SEQ ID NOs: 22, 36, and 38-41. In some aspects, the antibody comprises VH/VL sequences (respectively) comprising the amino acid sequences of SEQ ID NOs: 52/36 (h2A10.v2.N54Q). In some aspects, the antibody comprises a complete H chain having at least 90%, 91%, 92%, 93%, 94%, 95%, 96%, 97%, 98%, 99%, or 100% sequence identity to the amino acid sequence of SEQ ID NO: 74. In some aspects, the anti-TGFβ3 antibody comprises a complete L chain amino acid sequence having at least 90%, 91%, 92%, 93%, 94%, 95%, 96%, 97%, 98%, 99%, or 100% sequence identity to an amino acid sequence selected from the group consisting of SEQ ID NOs: 28, 58, and 60-63. In some aspects, the anti-TGFβ3 antibody comprises a complete H chain having at least 90%, 91%, 92%, 93%, 94%, 95%, 96%, 97%, 98%, 99%, or 100% sequence identity to the amino acid sequence of SEQ ID NO: 74 and a complete L chain amino acid sequence having at least 90%, 91%, 92%, 93%, 94%, 95%, 96%, 97%, 98%, 99%, or 100% sequence identity to an amino acid sequence selected from the group consisting of SEQ ID NOs: 28, 58, and 60-63. In some aspects, the antibody comprises complete H/L chain sequences (respectively) comprising the amino acid sequences of SEQ ID NOs: 74/58.

In another embodiment, an anti-TGFβ3 antibody is provided, wherein the antibody comprises a VH having at least 90%, 91%, 92%, 93%, 94%, 95%, 96%, 97%, 98%, 99%, or 100% sequence identity to the amino acid sequence of SEQ ID NO: 51 or 55. In some aspects, the anti-TGFβ3 antibody comprises a VL amino acid sequence having at least 90%, 91%, 92%, 93%, 94%, 95%, 96%, 97%, 98%, 99%, or 100% sequence identity to an amino acid sequence selected from the group consisting of SEQ ID NOs: 22, 36, 38-41, and 54. In some aspects, the anti-TGFβ3 antibody comprises VH/VL sequences (respectively) comprising the amino acid sequences of SEQ ID NO: 51/36 or SEQ ID NO: 55/54 (h2A10.v3). In some aspects, the antibody comprises a complete H chain having at least 95% sequence identity to the amino acid sequence of SEQ ID NO: 73 or 77 and/or the anti-TGFβ3 antibody comprises a complete L chain amino acid sequence having at least 90%, 91%, 92%, 93%, 94%, 95%, 96%, 97%, 98%, 99%, or 100% sequence identity to an amino acid sequence selected from the group consisting of SEQ ID NOs: 28, 58, 60-63, and 76. In some aspects, the antibody comprises complete H/L chain sequences (respectively) comprising the amino acid sequences of SEQ ID NOs: 73/58 or SEQ ID NOs: 77/76.

In another embodiment, an anti-TGFβ3 antibody is provided, wherein the antibody comprises a VH having at least 90%, 91%, 92%, 93%, 94%, 95%, 96%, 97%, 98%, 99%, or 100% sequence identity to the amino acid sequence of SEQ ID NO: 53 or 57. In some aspects, the anti-TGFβ3 antibody comprises a VL amino acid sequence having at least 90%, 91%, 92%, 93%, 94%, 95%, 96%, 97%, 98%, 99%, or 100% sequence identity to an amino acid sequence selected from the group consisting of SEQ ID NOs: 22, 36, 38-41, and 56. In some aspects, the anti-TGFβ3 antibody comprises a VH having at least 90%, 91%, 92%, 93%, 94%, 95%, 96%, 97%, 98%, 99%, or 100% sequence identity to the amino acid sequence of SEQ ID NO: 57. In other aspects, the anti-TGFβ3 antibody comprises a VL having at least 90%, 91%, 92%, 93%, 94%, 95%, 96%, 97%, 98%, 99%, or 100% sequence identity to the amino acid sequence of SEQ ID NO: 56. In some aspects, the anti-TGFβ3 antibody comprises a VH having at least 90%, 91%, 92%, 93%, 94%, 95%, 96%, 97%, 98%, 99%, or 100% sequence identity to the amino acid sequence of SEQ ID NO: 57, and a VL amino acid sequence having at least 90%, 91%, 92%, 93%, 94%, 95%, 96%, 97%, 98%, 99%, or 100% sequence identity to the amino acid sequence of SEQ ID NOs: 56. In some aspects, the anti-TGFβ3 antibody comprises VH/VL sequences (respectively) comprising the amino acid sequences of SEQ ID NOs: 53/36 or SEQ ID NOs: 57/56 (h2A10.v4). In some aspects, the anti-TGFβ3 antibody comprises VH/VL sequences (respectively) comprising the amino acid sequences of SEQ ID NOs: 57/56 (h2A10.v4). In some aspects, the antibody comprises a complete H chain having at least 95% sequence identity to the amino acid sequence of SEQ ID NO: 74 or 79 and/or a complete L chain amino acid sequence having at least 90%, 91%, 92%, 93%, 94%, 95%, 96%, 97%, 98%, 99%, or 100% sequence identity to an amino acid sequence selected from the group consisting of SEQ ID NOs: 28, 58, 60-63, 76, and 78. In some aspects, the anti-TGFβ3 antibody comprises a complete H chain having at least 90%, 91%, 92%, 93%, 94%, 95%, 96%, 97%, 98%, 99%, or 100% sequence identity to an amino acid sequence of SEQ ID NO: 79. In other aspects, the anti-TGFβ3 antibody comprises a complete L chain having at least 90%, 91%, 92%, 93%, 94%, 95%, 96%, 97%, 98%, 99%, or 100% sequence identity to an amino acid sequence of SEQ ID NO: 78. In still further aspects, the anti-TGFβ3 antibody comprises a complete H chain having at least 90%, 91%, 92%, 93%, 94%, 95%, 96%, 97%, 98%, 99%, or 100% sequence identity to an amino acid sequence of SEQ ID NO: 79; and comprises a complete L chain having at least 90%, 91%, 92%, 93%, 94%, 95%, 96%, 97%, 98%, 99%, or 100% sequence identity to an amino acid sequence of SEQ ID NO: 78. In some aspects, the antibody comprises a complete H chain having at least 95% sequence identity to the amino acid sequence of SEQ ID NO: 79 and/or a complete L chain amino acid sequence having at least 90%, 91%, 92%, 93%, 94%, 95%, 96%, 97%, 98%, 99%, or 100% sequence identity to an amino acid sequence of SEQ ID NO: 78. In some aspects, the anti-TGFβ3 antibody comprises complete H/L chain sequences (respectively) comprising the amino acid sequences of SEQ ID NOs: 75/58 or SEQ ID NOs: 79/78. In certain aspects, the anti-TGFβ3 antibody comprises complete H/L chain sequences (respectively) comprising the amino acid sequences of SEQ ID NOs: 79/78.

In a further aspect, the invention provides an anti-TGFβ3 antibody that binds to the same epitope as an anti-TGFβ3 antibody provided herein. For example, in certain embodiments, an antibody is provided that binds to the same epitope as the anti-TGFβ3 antibody comprising (a) HVR-H1 comprising the amino acid sequence of SEQ ID NO: 4; (b) HVR-H2 comprising the amino acid sequence of SEQ ID NO: 5; and (c) HVR-H3 comprising the amino acid sequence of SEQ ID NO:6. In one embodiment, an antibody is provided that binds to the same epitope as the anti-TGFβ3 antibody comprising (a) HVR-H1 comprising the amino acid sequence of SEQ ID NO: 4; (b) HVR-H2 comprising the amino acid sequence of SEQ ID NO: 34; and (c) HVR-H3 comprising the amino acid sequence of SEQ ID NO:6. In another embodiment, an antibody is provided that binds to the same epitope as the anti-TGFβ3 antibody comprising (a) HVR-H1 comprising the amino acid sequence of SEQ ID NO: 4; (b) HVR-H2 comprising the amino acid sequence of SEQ ID NO: 35; and (c) HVR-H3 comprising the amino acid sequence of SEQ ID NO:6. In another embodiment, an antibody is provided that binds to the same epitope as the anti-TGFβ3 antibody comprising (a) HVR-H1 comprising the amino acid sequence of SEQ ID NO: 4; (b) HVR-H2 comprising the amino acid sequence of SEQ ID NO: 159; and (c) HVR-H3 comprising the amino acid sequence of SEQ ID NO:6.

In another aspect, an anti-TGFβ3 antibody is provided that binds to the same epitope as the anti-TGFβ3 antibody comprising VH/VL sequences (respectively) selected from the group consisting of SEQ ID NOs: 53/36 (h2A10.v2.t56A), SEQ ID NOs: 57/56 (h2A10.v4), SEQ ID NOs: 51/36 (h2A10.v2.N54S), SEQ ID NOs: 55/54 (h2A10.v3), SEQ ID NOs: 52/36 (h2A10.v2.N54Q), SEQ ID NOs: 23/22 (rat 2A10), SEQ ID NOs: 37/36 (v1), SEQ ID NOs: 37/38 (v1.1), SEQ ID NOs: 37/39 (v1.2), SEQ ID NOs: 37/40 (v1.3), SEQ ID NOs: 37/41 (v1.4), SEQ ID NOs: 42/36 (v1.5), SEQ ID NOs: 43/36 (v1.6), SEQ ID NOs:44/36 (v1.7), SEQ ID NOs: 45/36 (v2), SEQ ID NOs: 45/38 (v2.1), SEQ ID NOs: 45/39 (v2.2), SEQ ID NOs: 45/40 (v2.3), SEQ ID NOs: 45/41 (v2.4), SEQ ID NOs: 46/36 (v2.5), SEQ ID NOs: 47/36 (v2.6), SEQ ID NOs: 48/36 (v2.7), SEQ ID NOs: 49/36 (v2.8), SEQ ID NOs: 50/36 (v2.9), SEQ ID NOs: 55/54 (v3), and SEQ ID NOs: 57/56 (v4). In another aspect, an anti-TGFβ3 antibody is provided that binds to the same epitope as the anti-TGFβ3 antibody comprising VH/VL sequences (respectively) selected from the group consisting of SEQ ID NOs: 53/36 (h2A10.v2.t56A), SEQ ID NOs: 57/56 (h2A10.v4), SEQ ID NOs: 51/36 (h2A10.v2.N54S), SEQ ID NOs: 55/54 (h2A10.v3), SEQ ID NOs: 52/36 (h2A10.v2.N54Q), SEQ ID NOs: 23/22 (rat 2A10), SEQ ID NOs: 37/36 (v1), SEQ ID NOs: 37/38 (v1.1), SEQ ID NOs: 37/39 (v1.2), SEQ ID NOs: 37/40 (v1.3), SEQ ID NOs: 37/41 (v1.4), SEQ ID NOs: 42/36 (v1.5), SEQ ID NOs: 43/36 (v1.6), SEQ ID NOs:44/36 (v1.7), SEQ ID NOs: 45/36 (v2), SEQ ID NOs: 45/38 (v2.1), SEQ ID NOs: 45/39 (v2.2), SEQ ID NOs: 45/40 (v2.3), SEQ ID NOs: 45/41 (v2.4), SEQ ID NOs: 46/36 (v2.5), SEQ ID NOs: 47/36 (v2.6), SEQ ID NOs: 48/36 (v2.7), SEQ ID NOs: 49/36 (v2.8), and SEQ ID NOs: 50/36 (v2.9).

In one embodiment, an anti-TGFβ3 antibody is provided that binds to the same epitope as the anti-TGFβ3 antibody comprising VH/VL sequences (respectively) of SEQ ID NOs: 57/56 (h2A10.v4).

In certain embodiments, an isolated anti-TGFβ3 antibody is provided that comprises an antigen binding domain that directly contacts amino acid residues R325, K331, W332, H334, E335, T387, I388, L389, Y391, V392, G393, R394, P396, K397, and V398 on human TGFβ3, wherein the anti-TGFβ3 antibody selectively neutralizes TGFβ3.

In some aspects, provided herein is an isolated anti-TGFβ3 antibody that comprises: heavy chain CDRs comprising CDR-H1, -H2 and -H3, wherein CDR-H1 has the amino acid sequence of SEQ ID NO: 4, CDR-H2 has the amino acid sequence of one of SEQ ID NOs: 5, 34, 35, and 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9. In some embodiments, the CDR-H2 has the amino acid sequence of SEQ ID NO: 35. In certain embodiments, the isolated anti-TGFβ3 antibody further comprises a VH having at least 90%, 91%, 92%, 93%, 94%, 95%, 96%, 97%, 98%, 99%, or 100% sequence identity to the amino acid sequence of SEQ ID NO: 57. In some embodiments, the anti-TGFβ3 antibody further comprises a VL having at least 90%, 91%, 92%, 93%, 94%, 95%, 96%, 97%, 98%, 99%, or 100% sequence identity to the amino acid sequence of SEQ ID NO: 56. In certain embodiments, the anti-TGFβ3 antibody further comprises a complete H chain having at least 90%, 91%, 92%, 93%, 94%, 95%, 96%, 97%, 98%, 99%, or 100% sequence identity to an amino acid sequence of SEQ ID NO: 79. In other embodiments, the anti-TGFβ3 antibody further comprises a complete L chain having at least 90%, 91%, 92%, 93%, 94%, 95%, 96%, 97%, 98%, 99%, or 100% sequence identity to an amino acid sequence of SEQ ID NO: 78.

In another embodiment, an isolated anti-TGFβ3 antibody is provided that selectively neutralizes TGFβ3 and has reduced toxicity in mice relative to the pan-TGFβ antibody 1D11. In some embodiments, the anti-TGFβ3 antibody provided herein has reduced toxicity in mice relative to the pan-TGFβ antibody 1D11 at a dose of 50 mg/kg. The 1D11 antibody is described in Lonning et al. (Current Pharmaceutical Biotechnology, 2011, 12, 2176-2189) and has the following amino acid sequences:

VH:
(SEQ ID NO: 160)
QVQLQQSGPELVRPGASVKLSCKASGYIFITYWMNWVKQRPGQGLEWIG
QIFPASGSTNYNEMFEGKATLTVDTSSSTAYMQLSSLTSEDSAVYYCAR
GDGNYALDAMDYWGQGTSVTVSS
VL:
(SEQ ID NO: 161)
DIVLTQSPASLAVSLGQRATISCRASESVDSYGNSFMHWYQQKSGQPPK
LLIYLASNLESGVPARFSGSGSRTDFTLTIDPVEADDAATYYCQQNNED
PLTFGAGTKLEIK

In a further aspect of the invention, an isoform-selective anti-TGFβ antibody according to any of the above embodiments (e.g., monospecific anti-TGFβ2 and anti-TGFβ3 antibodies, and dual-specific, anti-TGFβ2/3 antibodies described herein) is a monoclonal antibody, including a chimeric, humanized or human antibody. In one embodiment, an isoform-selective anti-TGFβ antibody is an antibody fragment, e.g., a Fv, Fab, Fab′, scFv, diabody, or F(ab′)2 fragment. In another embodiment, the antibody is a full-length antibody, e.g., an intact IgG1 or IgG4 antibody, preferably a human IgG1, and still more preferably a human IgG1 comprising an N297 mutation (EU numbering as in Kabat), e.g., N297A or N297G, preferably N297G, or other antibody class or isotype as defined herein.

In a further aspect, an isoform-selective anti-TGFβ antibody according to any of the above embodiments may incorporate any of the features, singly or in combination, as described in Sections 1-7 below:

### 1. Antibody Affinity

In certain embodiments, an antibody provided herein has a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸ M to 10⁻¹³ M, e.g., from 10⁻⁹ M to 10⁻¹³ M).

In one embodiment, K_{D} is measured by a radiolabeled antigen-binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J Mol. Biol. 293:865-881(1999)). To establish conditions for the assay, MICROTITER© multi-well plates (Thermo Scientific) are coated overnight with 5 g/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23 °C). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, K_{D} is measured using a BIACORE® surface plasmon resonance assay. For example, an assay using a BIACORE®-2000 or a BIACORE®-3000 (BIAcore, Inc., Piscataway, NJ) is performed at 25 °C with immobilized antigen CM5 chips at ~10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 g/ml (~0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25 °C at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ. See, e.g., Chen et al., J. Mol. Biol. 293:865-881 (1999). If the on-rate exceeds 106 M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25 °C of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### 2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

### 3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J Immunol., 147: 86 (1991).) Human antibodies generated via human B-cell hybridoma technology are also described in Li et al., Proc. it. Acad. Si. USA, 1033557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, NJ, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, NJ, 2003); Sidhu et al., J. Mol. Biol. 338(2): 299-310 (2004); Lee et al., J Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### 6. Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for a TGFβ isoform (e.g., TGFβ1, TGFβ2, TGFβ3, or TGFβ2/3) and the other is for any other antigen. In certain embodiments, one of the binding specificities is for one TGFβ isoform (e.g., TGFβ1, TGFβ2, or TGFβ3) and the other is for a different TGFβ isoform. In certain embodiments, bispecific antibodies may bind to two different epitopes within a single TGFβ isoform. Bispecific antibodies may also be used to localize cytotoxic agents to cells that express one or more TGFβ isoform(s). Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to one or more TGFβ isoform(s) (e.g., TGFβ1, TGFβ2, and/or TGFβ3) as well as another, different antigen (see, US 2008/0069820, for example).

### 7. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody.

Any combination of deletion, insertion, and substitution can be made to arrive at the final construct, provided that the final construct possesses the desired characteristics, e.g., antigen-binding.

### a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | | Exemplary | Preferred |
|---|---|---|---|
| Residue | | Substitutions | Substitutions |
| Ala | (A) | Val; Leu; Ile | Val |
| Arg | (R) | Lys; Gln; Asn | Lys |
| Asn | (N) | Gln; His; Asp, Lys; Arg | Gln |
| Asp | (D) | Glu; Asn | Glu |
| Cys | (C) | Ser; Ala | Ser |
| Gln | (Q) | Asn; Glu | Asn |
| Glu | (E) | Asp; Gln | Asp |
| Gly | (G) | Ala | Ala |
| His | (H) | Asn; Gln; Lys; Arg | Arg |
| Ile | (I) | Leu; Val; Met; Ala; Phe; Norleucine | Leu |
| Leu | (L) | Norleucine; Ile; Val; Met; Ala; Phe | Ile |
| Lys | (K) | Arg; Gln; Asn | Arg |
| Met | (M) | Leu; Phe; Ile | Leu |
| Phe | (F) | Trp; Leu; Val; Ile; Ala; Tyr | Tyr |
| Pro | (P) | Ala | Ala |
| Ser | (S) | Thr | Thr |
| Thr | (T) | Val; Ser | Ser |
| Trp | (W) | Tyr; Phe | Tyr |
| Tyr | (Y) | Trp; Phe; Thr; Ser | Phe |
| Val | (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

### b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

In certain embodiments, the N-glycosylation site in the CH2 domain of an isoform-selective anti-TGFβ antibody of the invention is mutated to prevent glycosylation. For example, an isoform-selective anti-TGFβ antibody with an aglycosylated Fc region can be made by mutagenizing the amino acid residue at position 297 as in the EU index in the CH2 domain of the Fc region (e.g., N297). In certain embodiments, the glycosylation in the CH2 domain of the Fc region can be eliminated by altering the glycosylation consensus site, i.e., Asn at position 297 followed by any amino acid residue (in the case of human IgG, Ser) and Thr. The glycosylation site can be altered by amino acid insertions, deletions, and/or substitutions. For example, one or more amino acid residues can be inserted between Asn and Ser or between Ser and Thr to alter the original glycosylation site, wherein the insertions do not regenerate an N-glycosylation site. In certain particular embodiments, the amino acid residue at position 297 as in the EU index (e.g., the N-glycosylated site in Fe) within the CH2 domain of human IgG Fc is mutated to abolish the glycosylation site. In certain particular embodiments, the amino acid residue at position 297 as in the EU index (e.g., N297) is changed to Gly, Ala, Gln, Asp, or Glu. In some particular embodiments, the amino acid residue at position 297 as in the EU index (e.g., N297) is changed to Gly or Ala. In other particular embodiments, the amino acid residue at position 297 as in the EU index (e.g., N297) is changed to Gly. In certain other embodiments, the amino acid residue at position 299 as in the EU index can be substituted with another amino acid, for example, Ala, Val, or Gly. In certain particular embodiments, the mutations that result in an aglycosylated Fc do not affect the structure and/or stability of the isoform-selective anti-TGFβ antibody.

In certain embodiments, an isoform-selective anti-TGFβ antibody of the invention comprises an Fc region in which the amino acid residue at position 297 as in the EU index in the CH2 domain is mutated. In certain embodiments, the amino acid residue at position 297 as in the EU index is changed to Gly or Ala, preferably to Gly. In certain other embodiments, the amino acid residue at position 297 as in the EU index is deleted. In other embodiments, the N-glycan attached to the wild type amino acid residue at position 297 as in the EU index (e.g., N297) can be removed enzymatically, e.g., by deglycosylation. Suitable glycolytic enzymes include without limitation, peptide-N-glycosidase (PNGase).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### c) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII only, whereas monocytes express Fc(RI, Fc(RII and Fc(RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).) In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

### d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### e) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### B. Recombinant Methods and Compositions

Antibodies may be produced using recombinant methods and compositions, e.g., as described in U.S. Pat. No. 4,816,567. In one embodiment, isolated nucleic acid encoding an isoform-selective anti-TGFβ antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an isoform-selective anti-TGFβ antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an isoform-selective anti-TGFβ antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, NJ, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J Gen Virol. 36:59 (1977); baby hamster kidney cells (BHK); mouse sertoli cells (TM4 cells as described, e.g., in Mather, Biol. Reprod. 23:243-251 (1980); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, NJ), pp. 255-268 (2003).

### C. Assays

Isoform-selective anti-TGFβ antibodies provided herein may be identified, screened for, or characterized for their physical/chemical properties and/or biological activities by various assays known in the art, and their crystal structures may be solved.

### 1. Binding Assays and Other Assays

In one aspect, an antibody of the invention is tested for its antigen binding activity, e.g., by known methods such as ELISA, Western blot, etc.

In one aspect, antibody binding is detected using enzyme immunoassay. This technique is well-known in the art. See, Schuurs and VanWeemen, Clinica Chimica Acta, 81:1-40 (1977). In this technique, enzymes are applied as labels on antigen or antibodies for identification and localization of the immunoreactants. Any method in which the extent of binding of enzyme-labeled antigen, or enzyme labeled-antibody to its immunoreactant is measured, is included in this invention. The variety of enzymes used, methods of linking enzymes to the immunological components, purification of the conjugates, as well as various assay principles and methods are well described Schuurs and VanWeemen, supra.

In one aspect, antibody binding affinity is determined using Surface Plasmon Resonance (SPR) measurement with a BIAcore™-T200 instrument. In this method, a series S CM5 biosensor chip with protein A is applied to capture the antibody to achieve approximately 100 response units (RU) on each flow cell, followed by injection of 3-fold serial dilutions of the target antigen in appropriate buffer (e.g., HBS-EP buffer) at 25 °C with a flow rate of 50 μl/min for kinetics measurement. Association rates (kₒₙ) and dissociation rates (k_{off}) may be calculated using a simple one-to-one Langmuir binding model (BIAcore T200 evaluation software version 2.0) to determine the equilibrium dissociation constant (K_{D}), calculated as the ratio k_{off}/kₒₙ.

In another aspect, competition assays may be used to identify an antibody that competes with an isoform-specific anti-TGFβ antibody described herein. In certain embodiments, such a competing antibody binds to the same epitope (e.g., a linear or a conformational epitope) that is bound by the 4A11, 6F12, or 2A10 antibodies and humanized variants thereof as described herein. Detailed exemplary methods for mapping an epitope to which an antibody binds are provided in Morris (1996) “Epitope Mapping Protocols,” in Methods in Molecular Biology vol. 66 (Humana Press, Totowa, NJ).

In an exemplary competition assay, an immobilized TGFβ isoform (e.g., TGFβ1, TGFβ2, or TGFβ3) is incubated in a solution comprising a first labeled antibody that binds to the TGFβ isoform (e.g., the 4A11, 6F12, or 2A10 antibody or a humanized variant thereof, e.g., as shown in Tables 5, 6, 7, 12, 13, 14, 19, 20, 23 and 24) and a second unlabeled antibody that is being tested for its ability to compete with the first antibody for binding to the TGFβ isoform. The second antibody may be present in a hybridoma supernatant. As a control, immobilized TGFβ isoform is incubated in a solution comprising the first labeled antibody but not the second unlabeled antibody. After incubation under conditions permissive for binding of the first antibody to the TGFβ isoform, excess unbound antibody is removed, and the amount of label associated with the immobilized TGFβ isoform is measured. If the amount of label associated with the immobilized TGFβ isoform is substantially reduced in the test sample relative to the control sample, then that indicates that the second antibody is competing with the first antibody for binding to the TGFβ isoform. See Harlow and Lane (1988) Antibodies: A Laboratory Manual ch.14 (Cold Spring Harbor Laboratory, Cold Spring Harbor, NY).

In another example, antibody binding and/or binding competition is assayed using a radioimmunoassay (RIA). RIA is a well-known technique. See, e.g., Chard, “An Introduction to Radioimmunoassy and Related Techniques”, North-Holland Publishing Company, 1978. Any of the many variations of RIA can be used, such as homogeneous phase RIA, or heterogeneous or solid phase RIA, single antibody methods or “double” antibody methods, and direct (forward) or reverse sandwich assays. Particularly preferred are solid phase systems wherein the is covalently coupled to an insoluble support so that both the antibody and the bound complex after incubation can be readily separated from the soluble free fraction. A wide variety of solid phase supports have been described, which include particles of dextran, cellulose, continuous surfaces such as polystyrene or polypropylene discs, walls of plastic tubes, glass discs, glass particles, and the like. Particulate solid phases are widely used for a variety of different assays and are included in the present invention. Antibodies are attached to the particles by any of a number of techniques designed to yield a non-reversible covalent or non-covalent link between protein and particle, for example directly or by cyanogen bromide activation. Other alternatives are the use of antibodies entrapped in the interstices of a polyacrylamide gel, or bound to magnetic particles. The assay tube is set up with sample or standard, tracer and the appropriate amount of solid phase bound antibody, plus a detergent to prevent aggregation of the particles and non-specific absorption of the tracer. After an incubation period during which the tubes are continuously mixed, the solid phase is sedimented by centrifugation; the supernatant is removed and the solid phase subject to two or more washes with buffer in order to remove free tracer trapped within and between the particles. The counts on the solid phase (bound fraction) are then measured. Immunoradiometric assays, Chards, supra at 423, can also be used.

### 2. Activity Assays

In one aspect, assays are provided for identifying isoform-selective anti-TGFβ antibodies thereof having biological activity. Biological activity may include, e.g., the ability to bind to and neutralize one or more TGFβ isoforms in vitro and/or in vivo.

In certain embodiments, an antibody of the invention is tested for such biological activity in an in vitro assay. In one embodiment, HEK-Blue™ TGFβ cells (InvivoGen, San Diego, CA) stably expressing human TGFβRI, Smad3, Smad4 proteins and secreted alkaline phosphatase (SEAP) reporter gene under the control of the β-globin minimal promoter fused to three Smad3/4-binding elements (SBE) may be used to evaluate the blocking potency of anti-TGFβ antibodies.

In another embodiment, the biological activity of anti-TGFβ antibodies of the invention may be assessed using the assay described in Abe et al. Analytical Biochemistry (1994); 216 (2): 276-284. Briefly, this assay uses a mink lung epithelial (MILE) cell-based TGFβ blocking assay in which the MILE cells are transfected with a plasmid containing the luciferase gene downstream of plasminogen activator inhibitor 1 promoter. The antibodies are incubated with each TGFβ isoform (PeproTech, Rocky Hill, NJ) at 25 °C for 1 h prior to the addition into 4×10⁴ MLE reporter cells seeded in 96-well plate in 75 μL of assay buffer (DMEM high glucose with 0.5% heat inactivated FBS, 2 mM L-glutamine and 0.5% Penicillin Streptomycin). The final concentration of antibody and TGFβ is in the range of 0.001-200 nM and 20 pM respectively. The plate is incubated at 37 °C and 5% CO2 for 20 h. The luciferase activity in cells is determined by adding Firefly Luciferase substrate according to the manufacturer's recommendations (Dual-Luciferase Reporter Assay System, Promega).

### D. Immunoconjugates

The invention also provides immunoconjugates comprising an isoform-selective anti-TGFβ antibody herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include At²¹¹I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-IBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

### E. Methods and Compositions for Diagnostics and Detection

In certain embodiments, any of the isoform-selective anti-TGFβ antibodies provided herein is useful for detecting the presence of one or more TGFβ isoforms (e.g., TGFβ1, TGFβ2, or TGFβ3) in a biological sample. The term “detecting” as used herein encompasses quantitative or qualitative detection. In certain embodiments, a biological sample comprises a cell or tissue, such as a cell or tissue from the lung, kidney, heart, pancreas, liver, or skin.

In one embodiment, an isoform-selective anti-TGFβ antibody for use in a method of diagnosis or detection is provided. In a further aspect, a method of detecting the presence of one or more TGFβ isoforms (e.g., TGFβ1, TGFβ2, or TGFβ3) in a biological sample is provided. In certain embodiments, the method comprises contacting the biological sample with an isoform-selective anti-TGFβ antibody as described herein under conditions permissive for binding of the isoform-selective anti-TGFβ antibody to one or more TGFβ isoforms (e.g., TGFβ1, TGFβ2, and/or TGFβ3), and detecting whether a complex is formed between the isoform-selective anti-TGFβ antibody and the TGFβ isoform(s). Such method may be an in vitro or in vivo method. In one embodiment, an anti-TGFβ antibody is used to select subjects eligible for therapy with an isoform-selective anti-TGFβ antibody, e.g., where the TGFβ isoform is a biomarker for selection of patients.

For example, it is demonstrated in Example 4, below, that TGFβ3 expression is markedly upregulated in SSc patients as compared to healthy controls, and further, that in qPCR analysis of bulk lung biopsy samples from patients with idiopathic pulmonary fibrosis (IPF), TGFβ3, and to a lesser extent, TGFβ2, were expressed at significantly higher levels compared to control lungs, whereas TGFβ1 expression was slightly lower on average in IPF compared to control. Thus in some embodiments, the expression levels of TGFβ isoforms, e.g., in a particular tissue, can be used to diagnose a patient with a condition of that tissue.

In some embodiments, the expression level of TGFβ2 and/or TGFβ3 can be used to identify patients that are likely to respond to treatment with an isoform-selective anti-TGFβ antibody of the invention, e.g., an anti-TGFβ2, anti-TGFβ2/3, or anti-TGFβ3 antibody as described herein. In some embodiments, a patient is determined to be more likely to respond to treatment with an isoform-selective anti-TGFβ antibody of the invention if the patient has elevated baseline level (prior to treatment with an isoform-selective anti-TGFβ antibody) relative to a reference level (e.g., healthy or control subject) of one or more or all of the biomarkers selected from TGFβ2, TGFβ3, periostin (POSTN) and cartilage oligomeric matrix protein (COMP). In some embodiments, such a patient has previously been administered an IL-6 inhibitor therapy, and has insufficient, minimal, or no response to said therapy. In some such embodiments, the IL-6 inhibitor is tocilizumab.

In some embodiments, the serum levels of the biomarkers (e.g., TGFβ2, TGFβ3, COMP and/or POSTN) are quantified in order to determine whether the patient is likely to respond to treatment with an isoform-selective anti-TGFβ antibody. In some embodiments, the patient is selected for treatment if the serum level of the one or more biomarkers (e.g., TGFβ2, TGFβ3, COMP and/or POSTN) is/are elevated at baseline relative to the reference level (e.g., in a healthy subject). In further embodiments, the patient that is identified as likely to respond to treatment with an isoform-selective anti-TGFβ antibody of the invention and/or that is selected for treatment is administered the isoform-selective anti-TGFβ antibody, e.g., in a therapeutically effective amount.

In certain embodiments, the serum levels of the biomarkers (e.g., TGFβ2, TGFβ3, COMP and/or POSTN) are quantified in order to determine that a patient is less likely to improve if the patient is administered a treatment comprising a therapeutic agent other than an isoform-selective anti-TGFβ antibody of the invention.

It is well within the skill in the art to measure levels of serum biomarkers, such as, e.g., TGFβ2, TGFβ3, POSTN and COMP. TGFβ isoform sequences have been provided, above. The nucleic and amino acid sequences of COMP and POSTN are known in the art and for reference, exemplary amino acid sequences are provided below:

The canonical amino acid sequence of human POSTN is:

(SEQ ID NO: 162)
MIPFLPMFSLLLLLIVNPINANNHYDKILAHSRIRGRDQGPNVCALQQI
LGTKKKYFSTCKNWYKKSICGQKTTVLYECCPGYMRMEGMKGCPAVLPI
DHVYGTLGIVGATTTQRYSDASKLREEIEGKGSFTYFAPSNEAWDNLDS
DIRRGLESNVNVELLNALHSHMINKRMLTKDLKNGMIIPSMYNNLGLFI
NHYPNGVVTVNCARIIHGNQIATNGVVHVIDRVLTQIGTSIQDFIEAED
DLSSFRAAAITSDILEALGRDGHFTLFAPTNEAFEKLPRGVLERIMGDK
VASEALMKYHILNTLQCSESIMGGAVFETLEGNTIEIGCDGDSITVNGI
KMVNKKDIVTNNGVIHLIDQVLIPDSAKQVIELAGKQQTTFTDLVAQLG
LASALRPDGEYTLLAPVNNAFSDDTLSMDQRLLKLILQNHILKVKVGLN
ELYNGQILETIGGKQLRVFVYRTAVCIENSCMEKGSKQGRNGAIHIFRE
IIKPAEKSLHEKLKystQDKRFSTFLSLLEAADLKELLTQPGDWTLFVP
TNDAFKGMTSEEKEILIRDKNALQNIILYHLTPGVFIGKGFEPGVTNIL
KTTQGSKIFLKEVNDTLLVNELKSKESDIMTTNGVIHVVDKLLYPADTP
VGNDQLLEILNKLIKYIQIKFVRGSTFKEIPVTVYTTKIITKVVEPKIK
VIEGSLQPIIKTEGPTLTKVKIEGEPEFRLIKEGETITEVIHGEPIIKK
YTKIIDGVPVEITEKETREERIITGPEIKYTRISTGGGETEETLKKLLQ
EEVTKVTKFIEGGDGHLFEDEEIKRLLQGDTPVRKLQANKKVQGSRRRL
REGRSQ.

The nucleic acid sequences and other variant amino acid sequences encoding human POSTN are known in the art and may be found in the Uniprot database accessible on the World Wide Web at uniprot.org/uniprot/Q15063 for Uniprot ID Q15063.

The canonical amino acid sequence of human COMP is:

(SEQ ID NO: 163)
MVPDTACVLLLTLAALGASGQGQSPLGSDLGPQMLRELQETNAALQDVR
ELLRQQVREITFLKNTVMECDACGMQQSVRTGLPSVRPLLHCAPGFCFP
GVACIQTESGARCGPCPAGFTGNGSHCTDVNECNAHPCFPRVRCINTSP
GFRCEACPPGYSGPTHQGVGLAFAKANKQVCTDINECETGQHNCVPNSV
CINTRGSFQCGPCQPGFVGDQASGCQRRAQRFCPDGSPSECHEHADCVL
ERDGSRSCVCAVGWAGNGILCGRDTDLDGFPDEKLRCPERQCRKDNCVT
VPNSGQEDVDRDGIGDACDPDADGDGVPNEKDNCPLVRNPDQRNTDEDK
WGDACDNCRSQKNDDQKDTDQDGRGDACDDDIDGDRIRNQADNCPRVPN
SDQKDSDGDGIGDACDNCPQKSNPDQADVDHDFVGDACDSDQDQDGDGH
QDSRDNCPTVPNSAQEDSDHDGQGDACDDDDDNDGVPDSRDNCRLVPNP
GQEDADRDGVGDVCQDDFDADKVVDKIDVCPENAEVTLTDFRAFQTVVL
DPEGDAQIDPNWVVLNQGREIVQTMNSDPGLAVGYTAFNGVDFEGTFHV
NTVTDDDYAGFIFGYQDSSSFYVVMWKQMEQTYWQANPFRAVAEPGIQL
KAVKSSTGPGEQLRNALWHTGDTESQVRLLWKDPRNVGWKDKKSYRWFL
QHRPQVGYIRVRFYEGPELVADSNVVLDTTMRGGRLGVFCFSQENIIWA
NLRYRCNDTIPEDYETHQLRQA.

The nucleic acid sequences and other variant amino acid sequences encoding human COMP are known in the art and may be found in the Uniprot database, accessible on the World Wide Web uniprot.org/uniprot/P49747 for Uniprot ID P49747.

Serum levels of POSTN and COMP may be determined by any suitable method known in the art, e.g., ELISA, microarray, or PCR. In another embodiment, protein levels of POSTN and COMP may be detected using IMPACT-based specific immunoassays, e.g., as described below, in Example 4. For example, whole blood may be collected from a subject or patient according to suitable methods in the art, allowed to clot, and then spun down. Serum biomarker levels are determined using IMPACT-based specific immunoassays (Roche Diagnostics, Penzberg, Germany). In brief, samples may be incubated with an IMPACT chip coated with biotinylated F(ab′)2 antibody fragments specific for a target protein of interest (e.g., a biomarker, such as, e.g., TGFβ2, TGFβ3, COMP or POSTN). After wash steps, biomarker-specific detection antibodies labelled with digoxigenin are added and incubated. After washing, fluorescence anti-digoxigenin-latex conjugate was added, and the specifically-bound fluorescence label may be detected with a charge-coupled detector camera. Emitted fluorescence is transformed into signal intensities using a standard curve composed of recombinant protein standards (R&D Systems, Minneapolis, MN, USA) and dedicated software (Roche Diagnostics).

Nucleic acid, according to any of the methods described herein may be RNA transcribed from genomic DNA or cDNA generated from RNA. Nucleic acid may be derived from a vertebrate, e.g., a mammal. A nucleic acid is said to be “derived from” a particular source if it is obtained directly from that source or if it is a copy of a nucleic acid found in that source.

Nucleic acid includes copies of the nucleic acid, e.g., copies that result from amplification. Amplification may be desirable in certain instances, e.g., in order to obtain a desired amount of material for detecting variations. The amplicons may then be subjected to a variation detection method, such as those described below, to determine expression of certain genes.

A microarray is a multiplex technology that typically uses an arrayed series of thousands of nucleic acid probes to hybridize with, e.g, a cDNA or cRNA sample under high-stringency conditions. Probe-target hybridization is typically detected and quantified by detection of fluorophore-, silver-, or chemiluminescence-labeled targets to determine relative abundance of nucleic acid sequences in the target. In typical microarrays, the probes are attached to a solid surface by a covalent bond to a chemical matrix (via epoxy-silane, amino-silane, lysine, polyacrylamide or others). The solid surface is for example, glass, a silicon chip, or microscopic beads. Various microarrays are commercially available, including those manufactured, for example, by Affymetrix, Inc. and Illumina, Inc.

In another embodiment, serum biomarker levels may be measured using the clinical trial version of the Elecsys (Roche Diagnostics) POSTN immunoassay intended for use on the cobas e601 (Roche Diagnostics).

In some embodiments, a method of determining disease burden and/or prognosis in a subject having a fibrotic condition is provided. In some non-limiting embodiments, the fibrotic condition is SSc or IPF. The method can include detecting the peripheral blood serum levels of POSTN and/or COMP in the subject.

Methods for detecting levels of proteins are known in the art. Expression levels of proteins may be detected in samples of whole blood, plasma, or serum. Various methods are known in the art for detecting protein expression levels in such biological samples, including various immunoassay methods. A wide range of immunoassay techniques have been previously described, see, e.g., U.S. Pat. Nos. 4,016,043, 4,424,279 and 4,018,653. These include both single-site and two-site or “sandwich” assays of the non-competitive types, as well as in the traditional competitive binding assays. These assays also include direct binding of a labeled antibody to a target biomarker.

Sandwich assays are among the most useful and commonly used assays. A number of variations of the sandwich assay technique exist, and all are intended to be encompassed by the present invention. Briefly, in a typical forward assay, an unlabeled antibody is immobilized on a solid substrate, and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of biomarker.

In other embodiments, the presence of elevated levels of one or more TGFβ isoforms can be used diagnostically to determine the presence or incipient presence of pathologies deriving from a TGFβ-related disorder (e.g., fibrosis or cancer). For example, immunoassays utilizing anti-TGFβ antibodies provide such a diagnostic test. Various formats of such assays are available and well known to those skilled in the art, including RIA, ELISA and immunofluorescence. See generally, Ruoslahti et al., M. Enz., 82:803-831 (1982). Alternatively, nucleic acid probes can be used to detect and quantitate TGFβ mRNA for the same purpose. Such methods are also well known in the art.

In some embodiments, the subject is monitored over time, and starting serum levels of the biomarkers are compared to the levels at later timepoints during treatment, to assess whether the subject's disease burden is changing and/or whether the subject's prognosis is improving or worsening. In some embodiments, a subject's condition and/or prognosis is less likely to improve if the expression level of one or both of POSTN and COMP is increased relative to a healthy control or reference standard, or relative to the subject's POSTN or COMP levels at the start of or prior to starting a treatment regimen for the TGFβ-related disorder, e.g., IPF or SSc.

In some aspects, a method of identifying a subject as one who has high TGFβ activity and/or as likely to benefit from treatment with an inhibitor of a TGFβ isoform, e.g., with an isoform-selective anti-TGFβ antibody of the invention (e.g., an anti-TGFβ2, anti-TGFβ2/3, or anti-TGFβ3 antibody as described herein), is provided. In some aspects the subject has SSc. In some aspects, the subject has IPF. In some aspects, the method comprises detecting the expression levels of one or more of the genes in an 18-gene signature set with the following genes: PRSS23, PXDN, COL8A1, COL6A3, SERPINE2, TNC, COMP, THBS1, COL11A1, COL1A1, COL5A2, COL1A2, COL4A1, COL4A2, SFRP4, ALPK2, COL5A1, TAGLN. The genes of the 18-gene signature set are described in Table 2, below. In some aspects, the method comprises identifying the subject as one who has high TGFβ activity and/or as likely to benefit from treatment with an inhibitor of a TGFβ isoform if the levels of 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, or all 18 of the genes in the 18-gene signature set are elevated relative to the levels of the gene in a healthy control set (e.g., a reference value). In one aspect, the method comprises identifying the subject as one having high TGFβ activity and/or as likely to benefit from treatment with an inhibitor of a TGFβ isoform if the level of each and all of the 18 genes in the 18-gene signature set is elevated relative to the levels of the respective gene in a healthy control set.

TABLE 2
18-gene signature set

| | | GenBank ® | SEQ |
|---|---|---|---|
| | Gene | Accession No. | ID |
| | Name | (nucleotide) | NOs: |
| 1 | PRSS23 | NM_007173.6 | 164 |
| 2 | PXDN | NM_012293.3 | 165 |
| 3 | COL8A1 | NM_001850.5 | 166 |
| 4 | COL6A3 | NM_004369.4 | 167 |
| 5 | SERPINE2 | NM_006216.3 | 168 |
| 6 | TNC | NM_002160.4 | 169 |
| 7 | COMP | NM_000095.3 | 170 |
| 8 | THBSI | NM_003246.4 | 171 |
| 9 | COL11A1 | NM_001854.4 | 172 |
| 10 | COL1A1 | NM_000088.4 | 173 |
| 11 | COL5A2 | NM_000393.5 | 174 |
| 12 | COL1A2 | NM_000089.4 | 175 |
| 13 | COL4A1 | NM_001845.6 | 176 |
| 14 | COL4A2 | NM_001846.4 | 177 |
| 15 | SFRP4 | NM_003014.4 | 178 |
| 16 | ALPK2 | NM_052947.4 | 179 |
| 17 | COL5A1 | NM_000093.5 | 180 |
| 18 | TAGLN | NM_001001522.2 | 181 |

In a specific embodiment, a subject is identified as one who has high TGFβ3 activity and/or as likely to benefit from treatment with an inhibitor of a TGFβ isoform if the levels of one or more of the genes selected from SERPINE1, COL1A1, COL1A2, and COUA1 are elevated relative to the level of the respective gene in a healthy control set (e.g., a reference value). The genes of this gene set are described in more detail in Table 3, below. In some aspects, the method comprises identifying the subject as one who has high TGFβ3 activity and/or as likely to benefit from treatment with an inhibitor of a TGFβ3 isoform if the levels of 1 or more, 2 or more, 3 or more, or all 4 of the genes SERPINE1, COL1A1, COL1A2, and COUA1 are elevated relative to the level of the respective gene in a healthy control set (e.g., a reference value).

TABLE 3
TGFβ Gene Signature

| | | GenBank ® | SEQ |
|---|---|---|---|
| | Gene | Accession No. | ID |
| | Name | (nucleotide) | NO: |
| 1 | SERPINE1 | NM_000602.5 | 182 |
| 2 | COL1A1 | NM_000088.4 | 173 |
| 3 | COL1A2 | NM_000089.4 | 175 |
| 4 | COL3A1 | NM_000090.4 | 183 |

In some embodiments of the above methods, the gene expression level is considered to be elevated if its increase in expression relative to the control or reference level of the gene is statistically significant. Methods for determining statistical significance are well-known in the art. By way of non-limiting example, statistical significance may be determined using, e.g., the unpaired two-tailed Student's t-test. In other embodiments, the gene expression level is considered to be elevated if its increase in expression relative to the control or reference level of the gene is at least 2-fold, at least 3-fold, or at least 4-fold.

In other aspects, methods of monitoring response to treatment of a subject with a TGFβ inhibitor, such as, e.g., an anti-TGFβ2 antibody and/or an anti-TGFβ3 antibody as described herein, are provided. The method includes determining the expression level of one or more of the TGFβ-inducible genes selected from PRSS23, PXDN, COL8A1, COL6A3, SERPINE2, TNC, COMP, THBS1, COL11A1, COL1A1, COL5A2, COL1A2, COL4A1, COL4A2, SFRP4, ALPK2, COL5A1, TAGLN and/or one or more of the genes selected from SERPINE1, COL1A1, COL1A2, and COL3A1. In some embodiments, the method further comprises stopping treatment with the TGFβ inhibitor if the expression levels of one or more of the genes selected from PRSS23, PXDN, COL8A1, COL6A3, SERPINE2, TNC, COMP, THBS1, COL11A1, COL1A1, COL5A2, COL1A2, COL4A1, COL4A2, SFRP4, ALPK2, COL5A1, TAGLN and/or one or more of the genes selected from SERPINE1, COL1A1, COL1A2, and COL3A1 are not decreased relative to the respective level at an earlier timepoint, e.g., prior to or at the beginning of treatment with the TGFβ inhibitor.

In another embodiment, a method of selecting a subject for treatment with a TGFβ inhibitor, such as, e.g., an anti-TGFβ2 antibody and/or an anti-TGFβ3 antibody as described herein is provided. The method can include detecting the level of one or both of serum POSTN and COMP in the subject, and administering the subject the TGFβ inhibitor if the levels of one or both of serum POSTN and COMP are elevated compared to a healthy control level. In some embodiments, the TGFβ inhibitor is an anti-TGFβ3 antibody, e.g., the antibody 2A10 or a humanized variant thereof, e.g., as shown in FIGS. 12-16 or Tables 5-7 in Example 1, below.

In some embodiments, the method of identifying the subject as one who has high TGFβ activity and/or as likely to benefit from treatment with an inhibitor of a TGFβ isoform, and/or for selecting a subject for treatment with a TGFβ inhibitor, comprises administering a therapy to the subject. In some embodiments, the therapy is for treating a TGFβ-related disorder, e.g., as described herein. In some embodiments, the therapy is for treating a fibrotic condition, e.g., as described herein. In some embodiments, the therapy comprises administering an anti-TGFβ2, anti-TGFβ2/3, or anti-TGFβ3 antibody as described herein. In a preferred embodiment, the therapy comprises administering an anti-TGFβ3 antibody as described herein, e.g., an isolated anti-TGFβ3 antibody, wherein the antibody selectively neutralizes TGFβ3, and wherein the antibody comprises one or more of the following features: (a) the anti-TGFβ3 antibody binds to the beta6/beta7 hairpin region of TGFβ3; (b) binding of the anti-TGFβ3 antibody sterically blocks the ability of TGFBR2, but not TGFBR1, to bind TGFβ3; (c) binding of the anti-TGFβ3 antibody to TGFβ3 blocks TGFBR2 binding and inhibits the TGFBR1/TGFBR2 signaling receptors from binding to TGFβ3; (d) the anti-TGFβ3 antibody binds amino acid residue R394 in the beta6/beta7 hairpin region of human TGFβ3; (e) the anti-TGFβ3 antibody binds amino acid residue R394 in the beta6/beta7 hairpin region of human TGFβ3 and residue R394 of TGFβ3 makes an ionic salt bridge with the anti-TGFβ3 antibody in the heavy chain CDR2; (f) isoform selectivity of the anti-TGFβ3 antibody for TGFβ3 over TGFβ1 is achieved by direct contact by the antigen binding domain of the anti-TGFβ3 antibody with amino acid residues T387, L389, and T395 of TGFβ3 (human TGFβ3 numbering); (g) the isoform selectivity of the anti-TGFβ3 antibody for TGFβ3 over TGFβ2 is achieved by direct contact by the antigen binding domain of the anti-TGFβ3 antibody with amino acid residues R325, R394, and V398 of TGFβ3 (human TGFβ3 numbering); (h) the anti-TGFβ3 antibody has reduced toxicity relative to the pan-TGFβ antibody 1D11; (i) the anti-TGFβ3 antibody has reduced toxicity in rodents or cynomolgus monkeys relative to the pan-TGFβ antibody 1D11; (j) the anti-TGFβ3 antibody has reduced toxicity relative to the pan-TGFβ small molecule inhibitor galunisertib; (k) the anti-TGFβ3 antibody has reduced toxicity in rodents relative to the pan-TGFβ small molecule inhibitor galunisertib; (l) the anti-TGFβ3 antibody has reduced toxicity relative to the anti-TGFβ1 antibody CAT-192; (m) the anti-TGFβ3 antibody has reduced toxicity relative to an isoform selective anti-TGFβ2 antibody and/or anti-TGFβ2/3 antibody; (n) the anti-TGFβ3 antibody comprises: (i) heavy chain CDRs comprising CDR-H1, -H2 and -H3, wherein CDR-H1 has the amino acid sequence of SEQ ID NO: 4, CDR-H2 has the amino acid sequence of one of SEQ ID NOs: 5, 34, 35, and 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9; (o) the anti-TGFβ3 antibody comprises an antigen binding domain that directly contacts amino acid residues R325, K331, W332, H334, E335, T387, I388, L389, Y391, V392, G393, R394, P396, K397, and V398 on human TGFβ3; and (p) the anti-TGFβ3 antibody as in (o), wherein the antigen binding domain is within 15-8, 8, 8-5, 7-5, 6-5, or 5 angstroms of the TGFβ3 amino acid residues.

In some aspects of the above embodiments, the anti-TGFβ3 antibody comprises a VH/VL pair, the VH/VL pair comprising amino acid sequences (respectively) selected from SEQ ID NOs: 23/22 (rat 2A10), SEQ ID NOs: 37/36 (v1), SEQ ID NOs: 37/38 (v1.1), SEQ ID NOs: 37/39 (v1.2), SEQ ID NOs: 37/40 (v1.3), SEQ ID NOs: 37/41 (v1.4), SEQ ID NOs: 42/36 (v1.5), SEQ ID NOs: 43/36 (v1.6), SEQ ID NOs:44/36 (v1.7), SEQ ID NOs: 45/36 (v2), SEQ ID NOs: 45/38 (v2.1), SEQ ID NOs: 45/39 (v2.2), SEQ ID NOs: 45/40 (v2.3), SEQ ID NOs: 45/41 (v2.4), SEQ ID NOs: 46/36 (v2.5), SEQ ID NOs: 47/36 (v2.6), SEQ ID NOs: 48/36 (v2.7), SEQ ID NOs: 49/36 (v2.8), and SEQ ID NOs: 50/36 (v2.9). In some embodiments, the anti-TGFβ3 antibody comprises a VH/VL pair comprising the amino acid sequences (respectively) of SEQ ID NOs: 52/36, or the amino acid sequences (respectively) of SEQ ID NOs: 51/36 or SEQ ID NOs: 55/54, or the amino acid sequences (respectively) of SEQ ID NOs: 53/36 or SEQ ID NOs: 57/56. In some embodiments, the anti-TGFβ3 antibody comprises a VH/VL pair comprising the amino acid sequences (respectively) of SEQ ID NOs: 57/56.

In some aspects of the above embodiments, the anti-TGFβ3 antibody comprises a complete H/L chain pair, the complete H/L chain pair comprising amino acid sequences (respectively) selected from SEQ ID NOs: 29/28 (rat 2A10), SEQ ID NOs: 59/58 (v1), SEQ ID NOs: 59/60 (v1.1), SEQ ID NOs: 59/61 (v1.2), SEQ ID NOs: 59/62 (v1.3), SEQ ID NOs: 59/63 (v1.4), SEQ ID NOs: 64/58 (v1.5), SEQ ID NOs: 65/58 (v1.6), SEQ ID NOs: 66/58 (v1.7), SEQ ID NOs: 67/58 (v2), SEQ ID NOs: 67/60 (v2.1), SEQ ID NOs: 67/61 (v2.2), SEQ ID NOs: 67/62 (v2.3), SEQ ID NOs: 67/63 (v2.4), SEQ ID NOs: 68/58 (v2.5), SEQ ID NOs: 69/58 (v2.6), SEQ ID NOs: 70/58 (v2.7), SEQ ID NOs: 71/58 (v2.8), SEQ ID NOs: 72/58 (v2.9), or the amino acid sequences (respectively) of SEQ ID NOs: 74/58, or the amino acid sequences (respectively) of SEQ ID NOs: 73/58 or SEQ ID NOs: 77/76, or the amino acid sequences (respectively) of SEQ ID NOs: 75/58 or SEQ ID NOs: 79/78. In some aspects, the anti-TGFβ3 antibody comprises a complete H/L chain pair comprising the amino acid sequences (respectively) of SEQ ID NOs: 79/78.

In some aspects of the above embodiments, the therapy comprises administering an anti-TGFβ2/3 antibody as described herein, e.g., an isolated anti-TGFβ2/3 antibody, wherein the antibody selectively neutralizes TGFβ2 and TGFβ3, wherein the antibody comprises one or more of the following features: (a) selectivity of the anti-TGFβ2/3 antibody for TGFβ2 and TGFβ3 over human TGFβ1, with respect to selective neutralization, is achieved by direct contact of the antibody's antigen binding domain with amino acid residue E373 of TGFβ2 or TGFβ3 (human TGFβ2 numbering); (b) the anti-TGFβ2/3 antibody neutralizes TGFβ2 and/or TGFβ3 via an allosteric mechanism; (c) the anti-TGFβ2/3 antibody induces a conformational change in TGFβ2 homodimer; (d) the anti-TGFβ2/3 antibody induces a conformational change in TGFβ2 homodimer, wherein the conformational change comprises the two monomers pinching together by several degrees; (e) the anti-TGFβ2/3 antibody is a divalent antibody or a monovalent antibody; (f) the anti-TGFβ2/3 antibody comprises (i) heavy chain CDRs comprising CDR-H1, -H2 and -H3, wherein CDR-H1 has the amino acid sequence of SEQ ID NO: 10, CDR-H2 has the amino acid sequence of SEQ ID NO: 11, and CDR-H3 has the amino acid sequence of SEQ ID NO: 12; and (ii) light chain CDRs comprising CDR-L1, -L2 and -L3, wherein CDR-L1 has the amino acid sequence of SEQ ID NO: 13; CDR-L2 has the amino acid sequence of SEQ ID NO: 14, and CDR-L3 has the amino acid sequence of SEQ ID NO: 15; (g) the anti-TGFβ2/3 antibody binds to TGFβ2 homodimer, the TGFβ2 homodimer having a first and a second TGFβ2 monomer, and wherein the anti-TGFβ2/3 antibody comprises an antigen binding domain that directly contacts (i) amino acid residues V313, Q314, D315, R320, L322, Y323, R328, D329, F345, and A347 of the first TGFβ2 monomer, and (ii) amino acid residues N368, T369, I370, N371, P372, E373, A374, S375, A376, and S377 of the second TGFβ2 monomer (human TGFβ2 numbering); (h) the anti-TGFβ2/3 antibody as in (g), wherein the antigen-binding domain is within 5 angstroms of the TGFβ2 and/or TGFβ3 amino acid residues; (i) wherein the anti-TGFβ2/3 antibody binds to the same epitope on TGFβ3 as in (g); and (j) the anti-TGFβ2/3 antibody does not neutralize TGFβ2 and/or TGFβ3 in single arm form.

In some aspects of the above embodiments, the anti-TGFβ2/3 antibody comprises a VH/VL pair, the VH/VL pair comprising amino acid sequences (respectively) selected from SEQ ID NOs: 27/26 (rabbit 4A11), SEQ ID NOs: 81/80 (v1), SEQ ID NOs: 81/82 (v2), SEQ ID NOs: 83/80 (v3), SEQ ID NOs: 83/82 (v4), SEQ ID NOs: 81/84 (v5), SEQ ID NOs: 81/85 (v6), SEQ ID NOs: 83/84 (v7), SEQ ID NOs: 86/84 (v7/1), SEQ ID NOs: 87/84 (v7.2), SEQ ID NOs: 88/84 (v7.3), SEQ ID NOs: 83/89 (v7.4), SEQ ID NOs: 83/90 (v7.5), SEQ ID NOs: 83/91 (v7.6), SEQ ID NOs: 83/92 (v7.7), SEQ ID NOs: 93/84 (v7.8), SEQ ID NOs: 94/84 (v7.9), SEQ ID NOs: 95/84 (v7.10), SEQ ID NOs: 96/84 (v7.11), SEQ ID NOs: 97/84 (v7.12), SEQ ID NOs: 98/84 (v7.13), SEQ ID NOs: 99/84 (v7.14), SEQ ID NOs: 100/84 (v7.15), SEQ ID NOs: 102/101 (v7.16), SEQ ID NOs: 103/101 (v7.17), SEQ ID NOs: 104/101 (v7.18), SEQ ID NOs: 105/101 (v7.19), and SEQ ID NOs: 83/85 (v8).

In some aspects of the above embodiments, the anti-TGFβ2/3 antibody comprises a complete H/L chain pair, the complete H/L chain pair comprising amino acid sequences (respectively) selected from SEQ ID NOs: 32/33 (rabbit 4A11), SEQ ID NOs: 107/106 (v1), SEQ ID NOs: 107/108 (v2), SEQ ID NOs: 109/106 (v3), SEQ ID NOs: 109/108 (v4), SEQ ID NOs: 107/110 (v5), SEQ ID NOs: 107/111 (v6), SEQ ID NOs: 109/110 (v7), SEQ ID NOs: 112/110 (v7.1), SEQ ID NOs: 113/110 (v7.2), SEQ ID NOs: 114/110 (v7.3), SEQ ID NOs: 114/115 (v7.4), SEQ ID NOs: 114/116 (v7.5), SEQ ID NOs: 114/117 (v7.6), SEQ ID NOs: 114/118 (v7.7), SEQ ID NOs: 119/110 (v7.8), SEQ ID NOs:120/110 (v7.9), SEQ ID NOs: 121/110 (v7.10), SEQ ID NOs: 122/110 (v7.11), SEQ ID NOs: 123/110 (v7.12), SEQ ID NOs: 124/110 (v7.13), SEQ ID NOs: 125/110 (v7.14), SEQ ID NOs: 126/110 (v7.15), SEQ ID NOs: 127/186 (v7.16), SEQ ID NOs: 128/186 (v7.17), SEQ ID NOs: 129/186 (v7.18), SEQ ID NOs: 130/186 (v7.19), and SEQ ID NOs: 114/111 (v8).

In some aspects of the above embodiments, the therapy comprises administering an anti-TGFβ2 antibody as described herein, e.g., an isolated anti-TGFβ2 antibody, wherein the antibody comprises: (a) heavy chain CDRs comprising CDR-H1, -H2 and -H3, wherein CDR-H1 has the amino acid sequence of SEQ ID NO: 16, CDR-H2 has the amino acid sequence of SEQ ID NO: 17, and CDR-H3 has the amino acid sequence of SEQ ID NO: 18; and (b) light chain CDRs comprising CDR-L1, -L2 and -L3, wherein CDR-L1 has the amino acid sequence of SEQ ID NO: 19; CDR-L2 has the amino acid sequence of SEQ ID NO: 20, and CDR-L3 has the amino acid sequence of SEQ ID NO: 21. In some aspects, the antibody selectively neutralizes TGFβ2. In some aspects, the anti-TGFβ2 antibody has one or more of the following properties: (a) selectively neutralizes TGFβ2; (b) has reduced toxicity relative to the pan-TGFβ antibody 1D11; (c) has reduced toxicity relative to the pan-TGFβ antibody 1D11; (d) has reduced toxicity in rodents relative to the pan-TGFβ antibody 1D11; (e) has reduced toxicity relative to the pan-TGFβ small molecule inhibitor galunisertib; and/or (f) has reduced toxicity in rodents relative to the pan-TGFβ small molecule inhibitor galunisertib.

In some aspects of the above embodiments, the anti-TGFβ2 antibody comprises a VH/VL pair, the VH/VL pair comprising amino acid sequences (respectively) selected from SEQ ID NOs: 25/24 (rabbit 6F12), SEQ ID NOs: 132/131 (v1), SEQ ID NOs: 132/133 (v1.1), SEQ ID NOs: 132/134 (v1.2), SEQ ID NOs: 132/135 (v1.3), SEQ ID NOs: 132/136 (v1.4), SEQ ID NOs: 132/137 (v1.5), SEQ ID NOs: 138/131 (v1.6), SEQ ID NOs: 139/131 (v1.7), SEQ ID NOs: 140/131 (v1.8), SEQ ID NOs: 141/131 (v1.9), SEQ ID NOs: 142/131 (v2), SEQ ID NOs: 132/143 (v3), and SEQ ID NOs: 142/144 (v4).

In some aspects of the above embodiments, the anti-TGFβ2 antibody comprises complete H/L chain pair, the complete H/L chain pair comprising amino acid sequences (respectively) selected from SEQ ID NOs: 31/30 (rabbit 6F12), SEQ ID NOs: 146/145 (v1), SEQ ID NOs: 146/147 (v1.1), SEQ ID NOs: 146/148 (v1.2), SEQ ID NOs: 146/149 (v1.3), SEQ ID NOs: 146/150 (v1.4), SEQ ID NOs: 146/151 (v1.5), SEQ ID NOs: 152/145 (v1.6), SEQ ID NOs: 153/145 (v1.7), SEQ ID NOs: 154/145 (v1.8), SEQ ID NOs: 155/145 (v1.9), SEQ ID NOs: 156/145 (v2), SEQ ID NOs: 146/157 (v3), and SEQ ID NOs: 156/158 (v4).

Exemplary conditions and disorders that may be diagnosed and/or treated according to the present invention, e.g., as described above, include TGFβ-related disorders, e.g., diseases characterized by accumulation of extracellular matrix, diseases caused by circulating TGFβ or TGFβ activated at a local site, including one or more TGFβ isoforms, conditions caused by suppression of the immune system due to endogenous TGFβ production, acute immune deficiencies resulting from severe injuries, burns, and illnesses such as viral or bacterial infections, multi-organ systemic illnesses due to TGFβ production or overproduction, and TGFβ-producing tumors. In some embodiments, disorders that may be diagnosed using an isoform-selective anti-TGFβ antibody of the invention includes fibrotic disease, e.g., as described herein. In some embodiments, the fibrotic disease is a fibrotic disorder of the lung, liver, heart, kidney, pancreas, eye, and/or skin. In some embodiments, the fibrosis is a fibrotic condition or disorder as described in Section G, below.

In some embodiments, a TGFβ-related disorder that may be diagnosed using an antibody of the invention is diagnosed using one or more of the antibodies 4A11, 6F12, and 2A10, or a humanized variant thereof, e.g., as described herein, e.g., in FIGS. 12-16 and 18-24.

In certain embodiments, labeled isoform-selective anti-TGFβ antibodies e.g., the antibodies described above, are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵, ³H, and ¹³¹I, fluorophores such as rare earth chelates or fluorescein and its derivatives, rhodamine and its derivatives, dansyl, umbelliferone, luciferases, e.g., firefly luciferase and bacterial luciferase (U.S. Pat. No. 4,737,456), luciferin, 2,3-dihydrophthalazinediones, horseradish peroxidase (RP), alkaline phosphatase, 0-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### F. Pharmaceutical Formulations

Pharmaceutical formulations of an isoform-selective anti-TGFβ antibody as described herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. For example, it may be desirable to further provide a therapeutic agent selected from pirfenidone, nintedanib, mycophenylate mofetil, an IL-6 inhibitor (e.g., tocilizumab, see, e.g., U.S. Pat. No. 9,539,263, sarilumab), an anti-CTFG antibody (e.g., FG-3019), an autotaxin inhibitor, a JAK inhibitor, an IL-11 inhibitor, and serum amyloid P (PTX2). (See, Raghu et al. European Respiratory Journal 2012 40: 2819) (FG-309, Ninou et al. (2018) Front Med (Lausanne); 5: 180 (autotaxin inhibitors).) Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

### G. Therapeutic Methods and Compositions

It is presently discovered that the isoform-selective anti-TGFβ antibodies described herein are useful in therapeutic methods. TGFβ has long been implicated as a key mediator of pathological fibrosis but its multifarious homeostatic functions have presented challenges to developing therapeutic TGFβ inhibitors with an acceptable therapeutic index. While the three TGFβ isoforms are capable of mediating similar signaling activities on target cells, their divergent LAP domains and expression patterns implicate unique roles for each isoform, supported by genetic loss-of-function evidence in mice and humans. While not intending to bound by any one particular theory or mechanism of action, it is discovered herein that the thresholds for and mechanisms of activation of latent TGFβ2 and TGFβ3 are distinct from those of TGFβ1 (see, e.g., Example 14); TGFβ2 and TGFβ3, unlike TGFβ1, are expressed at elevated levels in human fibrotic tissue and in mouse models of lung and liver; and isoform-selective therapeutic inhibition of TGFβ2 and TGFβ3 with a potent allosteric antibody can ameliorate experimental fibrosis in vivo. These findings support isoform-selective TGFβ2 and/or TGFβ3 inhibition as a therapeutic strategy for patients with chronic fibrotic disorders. For example, in Example 4, below, it is demonstrated that TGFβ2 and/or TGFβ3 expression levels are elevated in human fibrotic tissue and TGFβ3 expression is highly correlated with TGFβR signaling in IPF and SSc, two different fibrotic disorders, and in Example 5, describing a mouse model of bleomycin-induced lung fibrosis, in which neutralization of TGFβ2 with 6F12, neutralization of TGFβ3 with 2A10, and neutralization of both TGFβ2 and TGFβ3 with either the combination of 6F12+2A10 antibodies or monotherapy with dual-specific (anti-TGFβ2/3) 4A11 antibody reduced new collagen synthesis. Further, in skin biopsies taken at baseline in the FaSScinate trial (SSc of the skin, see, Khanna et al. (2016) The Lancet; vol. 387:10038, p. 2630-2640), it is presently demonstrated that TGFβ3 expression, as well as expression of a specific set of genes in a TGFβ signature, are markedly upregulated in SSc patients as compared to healthy controls, and that TGFβ3 expression correlated strongly and positively with the TGFβ signature, supporting inhibition of TGβ3 for the treatment of SSc using an anti-TGFβ3 antibody as described herein. Further, the data in Example 16 show that treatment with neutralizing antibodies to TGFβ2 and TGFβ3 can reduce lung fibrosis in vivo, and the data in Example 17 show that TGFβ2 and TGFβ3 inhibition can reduce hepatic fibrosis in vivo. Thus, these data support the inhibition of TGFβ2 and/or TGFβ3 for the treatment of TGFβ-related disorders, such as but not limited to fibrotic conditions.

Thus, in certain aspects, the isoform-selective anti-TGFβ antibodies are useful for the treatment of TGFβ-related disorders. Non-limiting specific examples of TGFβ-related disorders include neuronal, glial, astrocytal, hypothalamic and other glandular, macrophagal, epithelial, stromal and blastocoelic disorders, fibrosis, scarring, tissue damage such as caused by radiation, and adhesion during wound healing, fibrotic skin disorders such as scleroderma, CNS pathology scar tissue, dermal scarring, keloid scarring, and neural scarring, fibrotic diseases of the peritoneal cavity, lungs, liver, and kidneys such as chronic hepatic fibrosis, acute liver injury, interstitial lung and renal fibrosis, and liver cirrhosis, cystic fibrosis, vascular disorders, e.g., cardiac fibrosis, arterial injury such as atherosclerosis and arteriosclerosis, benign and malignant tumors, certain leukemias not inhibited by TGFβ, and malignancies (e.g., sarcomas, carcinomas, and melanomas), including prostate, fibrotic, ovarian, malignant melanoma, breast, lung, colon, rectal, colorectal, or cervical cancer and metastatic cancer, as well as neuroendocrine tumors of the digestive system and glioblastomas, angiopathy, vasculopathy, nephropathy, systemic sclerosis, infections such as macrophage pathogen infections and viral infections such as hepatitis C and HIV, immunological, angiogenic, and inflammatory disorders and deficiencies such as rheumatoid arthritis, an ocular disorder, especially those involving ocular fibrosis, including proliferative retinopathy, retinal detachment and post-glaucoma drainage surgery such as neural retina, retinal pigment epithelium-choroid and vitreous of the human eye, and cataracts, osteoporosis, adult respiratory distress syndrome, post-myocardial infarction, post-angioplasty restenosis, glomerulonephritis, a diabetes-related condition such as hyperglycemia, diabetes, diabetic kidney disease, diabetic nephropathy, diabetic neuropathy or retinopathy, and macrophage-deficiency diseases.

Preferably, the TGFβ-related disorder is fibrosis, an arterial injury, an infection, rheumatoid arthritis, diabetes or a diabetic condition, or a malignancy, such as cancer that expresses TGFβ, more preferably wherein the cancer is characterized by excessive activation of TGFβ. Such cancer may overexpress TGFβ, or alternatively not be characterized by overexpression of TGFβ.

In further aspects, the isoform-selective anti-TGFβ antibodies are useful for the treatment of fibrotic diseases, e.g., fibrotic diseases of the lung, liver, kidney, skin, heart, pancreas, gastrointestinal organs, and genitourinary organs; vascular tissue; and ocular tissue such as corneal tissue, retinal tissue, and lacrimal gland tissues. In some embodiments, the isoform-selective anti-TGFβ antibodies are useful for the treatment of diseases and disorders associated with aberrant TGFβ regulation and function, for example, and without limitation, pulmonary fibrosis, glomerulonephritis and diabetic kidney disease, congestive heart failure, liver cirrhosis, Marfan syndrome hypertrophic scars and SSc. In certain embodiments, the fibrotic condition is related to an autoimmune condition. In other embodiments, the fibrotic condition is subsequent to injury, including radiation, alkali burn, physical burn, surgery, physical trauma, or a combination thereof. See, Niessen et al., “On the nature of hypertrophic scars and keloids: a review,” Plast. Reconstr. 30 Surg. 104:1435-1458 (1999); Friedlander M., “Fibrosis and diseases of the eye,” J. Clin. Invest. 117:576-586 (2007); Noble et al., “Pulmonary fibrosis: patterns and perpetrators,” J. Clin. Invest. 122:2756-2762 (2012); Bahn R. S., “Graves' ophthalmopathy,” N. Engl. J. Med. 362:726-738 (2010); Hinz B., “Formation and function of the myofibroblast during tissue repair,” J. Invest. Dermatol. 127:526-537 (2007); Gauglitz et al., “Hypertrophic scarring and keloids: pathomechanisms and current and emerging treatment strategies,” Mol. Med. 17:113-125 (2011); Lehmann et al., “Immune mechanisms in thyroid eye disease,” Thyroid: official journal of the American Thyroid Association 18:959-965 (2008); Phan S. H., “The myofibroblast in pulmonary fibrosis,” Chest 122:286S-289S (2002); Kuriyan et al., “The eye and thyroid disease,” Curr. Opin. Ophthalmol. 19:499-506 (2008).

Exemplary fibrotic conditions that may be treated with the isoform-selective anti-TGFβ antibodies described herein are described in more detail below.

### 1. Fibrosis of the Lung

Exemplary fibrotic conditions of the lung (i.e., pulmonary fibrosis) include, but are not limited to, idiopathic pulmonary fibrosis (IPF); idiopathic pulmonary upper lobe fibrosis (Amitani disease); familial pulmonary fibrosis; pulmonary fibrosis secondary to systemic inflammatory diseases such as, rheumatoid arthritis, scleroderma, lupus, cryptogenic fibrosing alveolitis, chronic obstructive pulmonary disease (COPD) or chronic asthma; cystic fibrosis; non-specific interstitial pneumonia (NSIP); cryptogenic organizing pneumonia (COP); progressive massive fibrosis, a complication of coal worker's pneumoconiosis; scleroderma/systemic sclerosis (SSc, including limited cutaneous (IcSSc) and diffuse cutaneous (dcSSc) forms and SSc-associated interstitial lung disease (SSc-ILD)); bronchiolitis obliterans-organizing pneumonia; connective tissue disease-associated ILD (CT-ILD), progressive fibrosing ILD, hypersensitivity pneumonitis, pulmonary hypertension; pulmonary tuberculosis; silicosis; asbestosis; acute lung injury; and acute respiratory distress (ARD; including bacterial pneumonia induced, trauma-induced, and viral pneumonia-induced, ventilator-induced, non-pulmonary sepsis induced).

### 2. Fibrosis of the Liver

Exemplary fibrotic conditions of the liver (i.e., liver fibrosis) include, but are not limited to, liver cirrhosis due to all etiologies; congenital hepatic fibrosis; obesity; fatty liver; alcohol induced liver fibrosis; non-alcoholic steatohepatitis (NASH); biliary duct injury; primary biliary cirrhosis (PBC), primary sclerosing cholangitis (PSC); infection- or viral-induced liver fibrosis (e.g., chronic hepatitis B and C virus infections); cystic fibrosis; autoimmune hepatitis; necrotizing hepatitis; primary sclerosing cholangitis; hemochromatosis; disorders of the biliary tree; hepatic dysfunction attributable to infections.

### 3. Fibrosis of the Heart

Exemplary fibrotic conditions of the heart and/or pericardium (i.e., heart or pericardial fibrosis, or fibrosis of the associate vasculature) include, but are not limited to, endomyocardial fibrosis; cardiac allograft vasculopathy (CAV); myocardial infarction; atrial fibrosis; congestive heart failure; arteriosclerosis; atherosclerosis; vascular stenosis; myocarditis; congestive cardiomyopathy; coronary infarcts; varicose veins; coronary artery stenosis and other post-ischemic conditions; and idiopathic retroperitoneal fibrosis.

### 4. Fibrosis of the Kidney

Exemplary fibrotic conditions of the kidney (i.e., kidney fibrosis) include, but are not limited to, glomerulonephritis (including membranoproliferative, diffuse proliferative, rapidly progressive or sclerosing, post-infectious and chronic forms); diabetic glomerulosclerosis; focal segmental glomerulosclerosis; IgA nephropathy; diabetic nephropathy; ischemic nephropathy, tubulointerstitial kidney fibrosis, HIV-associated nephropathy; membrane nephropathy; glomerulonephritis secondary to systemic inflammatory diseases such as lupus, scleroderma and diabetes glomerulonephritis; idiopathic membranoproliferative glomerular nephritis; mesangial proliferative glomerulonephritis; crescentic glomerulonephritis; amyloidosis (which affects the kidney among other tissues); autoimmune nephritis; renal tubulointerstitial fibrosis; renal arteriosclerosis; Alport's syndrome; nephrotic syndrome; chronic renal failure; chronic kidney disease, periglomerular fibrosis/atubular glomeruli; combined apical emphysema and basal fibrosis syndrome (emphysema/fibrosis syndrome); glomerular hypertension; nephrogenic fibrosing dermatopathy; polycystic kidney disease; Fabry's disease and renal hypertension.

### 5. Fibrosis of the Pancreas

Exemplary fibrotic conditions of the pancreas (i.e., pancreatic fibrosis) include, but are not limited to, stromal remodeling pancreatitis and stromal fibrosis.

Exemplary fibrotic conditions of the gastrointestinal tract (i.e., GI tract fibrosis) include, but are not limited to, Crohn's disease; ulcerative colitis; collagenous colitis; colorectal fibrosis; villous atrophy; crypt hyperplasia; polyp formation; healing gastric ulcer; and microscopic colitis.

### 6. Fibrosis of the Eye

Exemplary fibrotic conditions of the eye include, but are not limited to, ocular fibrosis, ophthalmic fibrosis, proliferative vitreoretinopathy; vitreoretinopathy of any etiology; fibrosis associated with retinal dysfunction; fibrosis associated with wet or dry macular degeneration; scarring in the cornea and conjunctiva; fibrosis in the corneal endothelium; anterior subcapsular cataract and posterior capsule opacification; anterior segment fibrotic diseases of the eye; fibrosis of the corneal stroma (e.g., associated with corneal opacification); fibrosis of the trabecular network (e.g., associated with glaucoma); posterior segment fibrotic diseases of the eye; fibrovascular scarring (e.g., in retinal or choroidal vasculature of the eye); retinal fibrosis; epiretinal fibrosis; retinal gliosis; subretinal fibrosis (e.g., associated with age related macular degeneration); tractional retinal detachment in association with contraction of the tissue in diabetic retinopathy; congenital orbital fibrosis; lacrimal gland fibrosis; corneal subepithelial fibrosis; and Grave's ophthalmopathy.

### 7. Additional Fibrotic Disorders

Additional fibrotic disorders or fibrosis resulting from any one of the aforementioned conditions include, but are not limited to, spinal cord injury/fibrosis or central nervous system fibrosis such as fibrosis after a stroke, Duchenne muscular dystrophy, dupuytren's contracture, fibrosis associated with neurodegenerative disorder such as Alzheimer's disease or multiple sclerosis; vascular restenosis; uterine fibrosis; endometriosis; ovarian fibroids; Peyronie's disease; polycystic ovarian syndrome; disease related pulmonary apical fibrosis in ankylosing spondylitis; post-surgical adhesions, scarring, and fibrosis incident to microbial infections (e.g., bacterial, viral, parasitic, fungal etc.). Fibroses of the skin can include, for example, and without limitation, scleroderma, keloids, and morphea.

### Methods of Treatment

As used herein, treatment of fibrosis or fibrotic conditions, e.g., those described above, is meant to include disruption of the fibrotic processes so as to halt progression of the fibrotic condition, slow progression of the fibrotic condition, or cause regression of the fibrotic condition (i.e., improve the patient's state of health with respect to the degree of fibrosis in the affected tissue or organ). In certain embodiments, where treatment precedes onset of the fibrotic condition, i.e., treatment is performed prior to a known or an otherwise expected onset of fibrosis, then such treatment may include preventing development or onset of the fibrotic condition. Administration of the various active agents can therefore be carried out for a suitable duration to either control or halt progression of the fibrotic condition, or prevent onset thereof.

Thus, in some aspects, the invention provides a method for treating fibrosis, wherein the fibrosis is a fibrotic condition of the lung, liver, heart, kidney, pancreas, eye and/or skin. In one aspect, the fibrosis is a lung fibrosis selected from idiopathic pulmonary fibrosis (IPF); idiopathic pulmonary upper lobe fibrosis (Amitani disease); familial pulmonary fibrosis; pulmonary fibrosis secondary to systemic inflammatory diseases such as, rheumatoid arthritis, scleroderma, lupus, cryptogenic fibrosing alveolitis, chronic obstructive pulmonary disease (COPD) or chronic asthma; cystic fibrosis; non-specific interstitial pneumonia (NSIP); cryptogenic organizing pneumonia (COP); progressive massive fibrosis, a complication of coal worker's pneumoconiosis; scleroderma/systemic sclerosis (SSc, including limited cutaneous (IcSSc) and diffuse cutaneous (dcSSc) forms and SSc-associated interstitial lung disease (SSc-ILD)); bronchiolitis obliterans-organizing pneumonia; connective tissue disease-associated ILD (CT-ILD), progressive fibrosing ILD, hypersensitivity pneumonitis, pulmonary hypertension; pulmonary tuberculosis; silicosis; asbestosis; acute lung injury; and acute respiratory distress (ARD; including bacterial pneumonia induced, trauma-induced, and viral pneumonia-induced, ventilator-induced, non-pulmonary sepsis induced). In another aspect, the fibrosis is a fibrotic condition of the liver selected from liver cirrhosis due to all etiologies; congenital hepatic fibrosis; obesity; fatty liver; alcohol induced liver fibrosis; non-alcoholic steatohepatitis (NASH); biliary duct injury; primary biliary cirrhosis (PBC), primary sclerosing cholangitis (PSC); infection- or viral-induced liver fibrosis (e.g., chronic hepatitis B and C virus infections); cystic fibrosis; autoimmune hepatitis; necrotizing hepatitis; primary sclerosing cholangitis; hemochromatosis; disorders of the biliary tree; hepatic dysfunction attributable to infections. In another aspect, the fibrosis is a fibrotic condition of the heart and/or pericardium (i.e., heart or pericardial fibrosis, or fibrosis of the associate vasculature) selected from endomyocardial fibrosis; cardiac allograft vasculopathy (CAV); myocardial infarction; atrial fibrosis; congestive heart failure; arteriosclerosis; atherosclerosis; vascular stenosis; myocarditis; congestive cardiomyopathy; coronary infarcts; varicose veins; coronary artery stenosis and other post-ischemic conditions; and idiopathic retroperitoneal fibrosis. In another aspect, the fibrosis is a fibrotic condition of the kidney selected from glomerulonephritis (including membranoproliferative, diffuse proliferative, rapidly progressive or sclerosing, post-infectious and chronic forms); diabetic glomerulosclerosis; focal segmental glomerulosclerosis; IgA nephropathy; diabetic nephropathy; ischemic nephropathy, tubulointerstitial kidney fibrosis, HIV-associated nephropathy; membrane nephropathy; glomerulonephritis secondary to systemic inflammatory diseases such as lupus, scleroderma and diabetes glomerulonephritis; idiopathic membranoproliferative glomerular nephritis; mesangial proliferative glomerulonephritis; crescentic glomerulonephritis; amyloidosis (which affects the kidney among other tissues); autoimmune nephritis; renal tubulointerstitial fibrosis; renal arteriosclerosis; Alport's syndrome; nephrotic syndrome; chronic renal failure; chronic kidney disease, periglomerular fibrosis/atubular glomeruli; combined apical emphysema and basal fibrosis syndrome (emphysema/fibrosis syndrome); glomerular hypertension; nephrogenic fibrosing dermatopathy; polycystic kidney disease; Fabry's disease and renal hypertension. In another aspect, the fibrosis is a fibrotic condition of the pancreas selected from stromal remodeling pancreatitis and stromal fibrosis. In another aspect, the fibrosis is a fibrotic condition of the gastrointestinal tract (i.e., GI tract fibrosis) selected from Crohn's disease; ulcerative colitis; collagenous colitis; colorectal fibrosis; villous atrophy; crypt hyperplasia; polyp formation; healing gastric ulcer; and microscopic colitis. In another aspect, the fibrosis is a fibrotic condition of the eye selected from ocular fibrosis, ophthalmic fibrosis, proliferative vitreoretinopathy; vitreoretinopathy of any etiology; fibrosis associated with retinal dysfunction; fibrosis associated with wet or dry macular degeneration; scarring in the cornea and conjunctiva; fibrosis in the corneal endothelium; anterior subcapsular cataract and posterior capsule opacification; anterior segment fibrotic diseases of the eye; fibrosis of the corneal stroma (e.g., associated with corneal opacification); fibrosis of the trabecular network (e.g., associated with glaucoma); posterior segment fibrotic diseases of the eye; fibrovascular scarring (e.g., in retinal or choroidal vasculature of the eye); retinal fibrosis; epiretinal fibrosis; retinal gliosis; subretinal fibrosis (e.g., associated with age related macular degeneration); tractional retinal detachment in association with contraction of the tissue in diabetic retinopathy; congenital orbital fibrosis; lacrimal gland fibrosis; corneal subepithelial fibrosis; and Grave's ophthalmopathy. In another aspect, the fibrosis is a selected from fibrosis resulting from spinal cord injury/fibrosis or central nervous system fibrosis such as fibrosis after a stroke, Duchenne muscular dystrophy, dupuytren's contracture, fibrosis associated with neurodegenerative disorder such as Alzheimer's disease or multiple sclerosis; vascular restenosis; uterine fibrosis; endometriosis; ovarian fibroids; Peyronie's disease; polycystic ovarian syndrome; disease related pulmonary apical fibrosis in ankylosing spondylitis; scarring; multi-organ connective tissue dysfunction involving tissues in skin, lung, heart, kidney, and/or intestine, e.g., systemic sclerosis (SSc), SSc of the skin, and fibrosis incident to microbial infections (e.g., bacterial, viral, parasitic, fungal etc.).

In one embodiment, the invention provides a method for treating a TGFβ-related disorder, wherein the method includes administering to a subject having such TGFβ-related disorder an effective amount of an isoform-selective anti-TGFβ antibody (e.g., anti-TGFβ2, anti-TGFβ2/3, or anti-TGFβ3 antibody). In one such embodiment, the method further comprises administering to the subject an effective amount of at least one additional therapeutic agent, as described below. In another embodiment, the invention provides a method for treating fibrosis (e.g., a fibrotic condition as described above), wherein the method includes administering to a subject having such fibrosis an effective amount of an isoform-selective anti-TGFβ antibody (e.g., anti-TGFβ2, anti-TGFβ2/3, or anti-TGFβ3 antibody). In one such embodiment, the method further comprises administering to the subject an effective amount of at least one additional therapeutic agent, as described below. In a further embodiment, the invention provides a method for inhibiting TGFBR-dependent SMAD signaling, for inhibiting the assembly of TGFβ-TGFBR signaling complexes, for inhibiting TGFβ signaling through the TGFBR1/R2 complex, for inhibiting TGFβ signaling through the TGFBR2/ALK1 complex facilitated by endoglin, and/or for inhibiting new collagen synthesis in a subject. In another embodiment, the method comprises administering to the subject an effective amount of an isoform-selective anti-TGFβ antibody (e.g., anti-TGFβ2, anti-TGFβ2/3, or anti-TGFβ3 antibody) to inhibit TGFBR-dependent SMAD signaling, to inhibit the assembly of TGFβ-TGFBR signaling complexes, to inhibit TGFβ signaling through the TGFBR1/R2 complex, to inhibit TGFβ signaling through the TGFBR2/ALK1 complex facilitated by endoglin and/or for inhibiting new collagen synthesis. Exemplary embodiments using the isoform-selective anti-TGFβ antibodies of the invention are provided, below.

### Methods of Treating Idiopathic Pulmonary Fibrosis

The present invention provides methods of treating idiopathic pulmonary fibrosis (IPF). The methods generally involve administering to an individual having IPF an effective amount of an isoform-selective anti-TGFβ antibody described herein (e.g., anti-TGFβ2, anti-TGFβ2/3, or anti-TGFβ3 antibody). In some embodiments, the dosing and efficacy of the treatment can be monitored by reversal or slowing of progressing of usual interstitial pneumonia (UIP) on histopathological evaluation of lung tissue obtained by surgical biopsy. The criteria for a diagnosis of IPF are known. Ryu et al. (1998) Mayo Clin. Proc. 73:1085-1101. In other embodiments, a diagnosis of IPF is a definite or probable IPF made by high resolution computer tomography (HRCT). In a diagnosis by HRCT, the presence of the following characteristics is noted: (1) presence of reticular abnormality and/or traction bronchiectasis with basal and peripheral predominance; (2) presence of honeycombing with basal and peripheral predominance; and (3) absence of atypical features such as micronodules, peribronchovascular nodules, consolidation, isolated (non-honeycomb) cysts, ground glass attenuation (or, if present, is less extensive than reticular opacity), and mediastinal adenopathy (or, if present, is not extensive enough to be visible on chest x-ray). A diagnosis of definite IPF is made if characteristics (1), (2), and (3) are met. A diagnosis of probable IPF is made if characteristics (1) and (3) are met. In certain preferred embodiments, the treatment results in an increase, such as a statistically significant increase, in pulmonary function. Pulmonary function values are well known in the art. The following is an example of pulmonary function values that may be used. Other pulmonary function values, or combinations thereof, are intended to be within the scope of this invention. The values include, but are not limited to, FEV (forced expiratory volume), FVC (forced vital capacity), FEF (forced expiratory flow), Vmax (maximum flow), PEFR (peak expiratory flow rate), FRC (functional residual capacity), RV (residual volume), TLC (total lung capacity). FEV measures the volume of air exhaled over a pre-determined period of time by a forced expiration immediately after a full inspiration. FVC measures the total volume of air exhaled immediately after a full inspiration. Forced expiratory flow measures the volume of air exhaled during a FVC divided by the time in seconds. Vmax is the maximum flow measured during FVC. PEFR measures the maximum flow rate during a forced exhale starting from full inspiration. RV is the volume of air remaining in the lungs after a full expiration.

### Methods of Treating Liver Fibrosis

The present invention provides methods of treating liver fibrosis, including reducing clinical liver fibrosis, reducing the likelihood that liver fibrosis will occur, and reducing a parameter associated with liver fibrosis. Of particular interest in many embodiments is treatment of humans. Liver fibrosis is a precursor to the complications associated with liver cirrhosis, such as portal hypertension, progressive liver insufficiency, and hepatocellular carcinoma. A reduction in liver fibrosis thus reduces the incidence of such complications. Accordingly, the present invention further provides methods of reducing the likelihood that an individual will develop complications associated with cirrhosis of the liver by conjoint therapy involving the administration of an effective amount of an isoform-selective anti-TGFβ antibody described herein (e.g., anti-TGFβ2, anti-TGFβ2/3, or anti-TGFβ3 antibody). Whether treatment with an effective amount of an isoform-selective anti-TGFβ antibody described herein (e.g., anti-TGFβ2, anti-TGFβ2/3, or anti-TGFβ3 antibody) is effective in reducing liver fibrosis is determined by any of a number of well-established techniques for measuring liver fibrosis and liver function. Whether liver fibrosis is reduced is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems. The METAVIR scoring system is based on an analysis of various features of a liver biopsy, including fibrosis (portal fibrosis, centrilobular fibrosis, and cirrhosis); necrosis (piecemeal and lobular necrosis, acidophilic retraction, and ballooning degeneration); inflammation (portal tract inflammation, portal lymphoid aggregates, and distribution of portal inflammation); bile duct changes; and the Knodell index (scores of periportal necrosis, lobular necrosis, portal inflammation, fibrosis, and overall disease activity). The definitions of each stage in the METAVIR system are as follows: score: 0, no fibrosis; score: 1, stellate enlargement of portal tract but without septa formation; score: 2, enlargement of portal tract with rare septa formation; score: 3, numerous septa without cirrhosis; and score: 4, cirrhosis. Knodell's scoring system, also called the Hepatitis Activity Index, classifies specimens based on scores in four categories of histologic features: I. Periportal and/or bridging necrosis; II. Intralobular degeneration and focal necrosis; III. Portal inflammation; and IV. Fibrosis. In the Knodell staging system, scores are as follows: score: 0, no fibrosis; score: 1, mild fibrosis (fibrous portal expansion); score: 2, moderate fibrosis; score: 3, severe fibrosis (bridging fibrosis); and score: 4, cirrhosis. The higher the score, the more severe the liver tissue damage. Knodell (1981) Hepatol. 1:431. In the Scheuer scoring system scores are as follows: score: 0, no fibrosis; score: 1, enlarged, fibrotic portal tracts; score: 2, periportal or portal-portal septa, but intact architecture; score: 3, fibrosis with architectural distortion, but no obvious cirrhosis; score: 4, probable or definite cirrhosis. Scheuer (1991) J. Hepatol. 13:372. The Ishak scoring system is described in Ishak (1995) J. Hepatol. 22:696-699. Stage 0, No fibrosis; Stage 1, Fibrous expansion of some portal areas, with or without short fibrous septa; stage 2, Fibrous expansion of most portal areas, with or without short fibrous septa; stage 3, Fibrous expansion of most portal areas with occasional portal to portal (P--P) bridging; stage 4, Fibrous expansion of portal areas with marked bridging (P--P) as well as portal-central (P--C); stage 5, Marked bridging (P-- and/or P--C) with occasional nodules (incomplete cirrhosis); stage 6, Cirrhosis, probable or definite. The benefit of anti-fibrotic therapy can also be measured and assessed by using the Child-Pugh scoring system which comprises a multicomponent point system based upon abnormalities in serum bilirubin level, serum albumin level, prothrombin time, the presence and severity of ascites, and the presence and severity of encephalopathy. Based upon the presence and severity of abnormality of these parameters, patients may be placed in one of three categories of increasing severity of clinical disease: A, B, or C.

### Methods of Treating Renal Fibrosis

Renal fibrosis is characterized by the excessive accumulation of extracellular matrix (ECM) components. Overproduction of TGFβ is believed to underly tissue fibrosis caused by excess deposition of ECM, resulting in disease. The fibrogenic action of TGFβ results from simultaneous stimulation of matrix protein synthesis, inhibition of matrix degradation and enhanced integrin expression that facilitates ECM assembly. The present invention provides methods of treating renal fibrosis. The methods generally involve administering to an individual having renal fibrosis an isoform-selective anti-TGFβ antibody described herein (e.g., anti-TGFβ2, anti-TGFβ2/3, or anti-TGFβ3 antibody). As used herein, an “effective amount” of an isoform-selective anti-TGFβ antibody described herein (e.g., anti-TGFβ2, anti-TGFβ2/3, or anti-TGFβ3 antibody) is a dosage that is effective in reducing renal fibrosis; and/or that is effective in reducing the likelihood that an individual will develop renal fibrosis; and/or that is effective in reducing a parameter associated with renal fibrosis; and/or that is effective in reducing a disorder associated with fibrosis of the kidney. In one embodiment, an effective amount of an isoform-selective anti-TGFβ antibody described herein (e.g., anti-TGFβ2, anti-TGFβ2/3, or anti-TGFβ3 antibody) is an amount that is sufficient to reduce renal fibrosis by at least about 10%, at least about 15%, at least about 20%, at least about 25%, at least about 30%, at least 20 about 35%, at least about 40%, at least about 45%, at least about 50%, compared to the degree of renal fibrosis in the individual prior to treatment with the isoform-selective anti-TGFβ antibody. Whether fibrosis is reduced in the kidney is determined using any known method. For example, histochemical analysis of kidney biopsy samples for the extent of ECM deposition and/or fibrosis is performed. Other methods are known in the art. See, e.g., Masseroli et al. (1998) Lab. Invest. 78:511-522; U.S. Pat. No. 6,214,542. In some embodiments, an effective amount of an isoform-selective anti-TGFβ antibody described herein (e.g., anti-TGFβ2, anti-TGFβ2/3, or anti-TGFβ3 antibody) is an amount that is effective to increase kidney function by at least about 10%, at least about 15%, at least about 20%, at least about 25%, at 30 least about 30%, at least about 35%, at least about 40%, at least about 45%, at least about 50%, compared to the basal level of kidney function in the individual prior to treatment with the isoform-selective anti-TGFβ antibody (e.g., anti-TGFβ2, anti-TGFβ2/3, or anti-TGFβ3 antibody) of the present invention. In some embodiments, an effective amount of the isoform-selective anti-TGFβ antibody is the amount that is effective to slow the decline in kidney function by at least about 10%, at least about 15%, at least about 20%, at least about 25%, at least about 30%, at least about 35%, at least about 40%, at least about 45%, at least about 50%, compared to the decline in kidney function that would occur in the absence of treatment with the isoform-selective anti-TGFβ antibody of the present invention. Kidney function can be measured using any known assay, including, but not limited to, plasma creatinine level (where normal levels are generally in a range of from about 0.6 to about 1.2 mg/dL); creatinine clearance (where the normal range for creatinine clearance is from about 97 to about 137 mL/minute in men, and from about 88 to about 128 mL/minute in women); the glomerular filtration rate (either calculated or obtained from inulin clearance or other methods), blood urea nitrogen (where the normal range is from about 7 to about 20 mg/dL); and urine protein levels. The invention also provides a method for treatment of renal fibrosis in an individual comprising administering to the individual an isoform-selective anti-TGFβ antibody in an amount that is effective for prophylaxis or therapy of renal fibrosis in the individual, e.g., increasing the time to doubling of serum creatinine levels, increasing the time to end-stage renal disease requiring renal replacement therapy (e.g., dialysis or transplant), increasing the probability of survival, reducing the risk of death, ameliorating the disease burden or slowing the progression of disease in the individual, while reducing the incidence or severity of one or more side effects that would ordinarily arise without such treatment.

### Anti-TGFβ2 Antibodies

In some aspects of the above methods for treating a TGFβ-related disorder, such as but not limited to fibrosis (e.g., fibrosis of the lung, liver, pancreas, heart, kidney, eye, and/or skin) are provided, the method comprises administering to a subject having such TGFβ-related disorder an effective amount of an anti-TGFβ2 antibody of the invention having one or more of the following properties:
the anti-TGFβ2 antibody:
(a) selectively neutralizes TGFβ2;
(b) has reduced toxicity relative to the pan-TGFβ antibody 1D11;
(c) has reduced toxicity relative to the pan-TGFβ antibody 1D11;
(d) has reduced toxicity in rodents relative to the pan-TGFβ antibody 1D11;
(e) has reduced toxicity relative to the pan-TGFβ small molecule inhibitor galunisertib; and/or
(f) has reduced toxicity in rodents relative to the pan-TGFβ small molecule inhibitor galunisertib.

In another aspect, the invention provides a method for treating a fibrosis condition, e.g., such as a fibrotic condition described above, the method comprising administering to a subject having such fibrosis an effective amount of an isoform-selective anti-TGFβ antibody having one or more of the following properties:
the anti-TGFβ2 antibody selectively neutralizes TGFβ2; the anti-TGFβ2 binds to TGFβ2 with a K_{D} of less than 10 pM and/or has a cell-based IC₅₀ of less than 250 pM; binds to TGFβ2 with a K_{D} of less than or equal to about 5 pM, about 4 pM, about 3 pM, about 2 pM, or about 1 pM; the anti-TGFβ2 antibody provided herein has a cell-based IC₅₀ less than or equal to about 250 pM, about 200 pM, about 150 pM, about 100 pM, about 75 pM, about 50 pM, or about 40 pM.

In one aspect, the invention provides a method for treating fibrosis, the method comprising administering to a subject having such fibrosis an effective amount of an isoform-selective anti-TGFβ antibody selected from the following:
(i) an anti-TGFβ2 antibody comprising at least one, two, three, four, five, or six HVRs selected from (a) HVR-H1 comprising the amino acid sequence of SEQ ID NO: 16; (b) HVR-H2 comprising the amino acid sequence of SEQ ID NO:17; (c) HVR-H3 comprising the amino acid sequence of SEQ ID NO: 18; (d) HVR-L1 comprising the amino acid sequence of SEQ ID NO: 19; (e) HVR-L2 comprising the amino acid sequence of SEQ ID NO: 20; and (f) HVR-L3 comprising the amino acid sequence of SEQ ID NO: 21;
(ii) an anti-TGFβ2 antibody comprising at least one, at least two, or all three VH HVR sequences selected from (a) HVR-H1 comprising the amino acid sequence of SEQ ID NO: 16; (b) HVR-H2 comprising the amino acid sequence of SEQ ID NO: 17; (c) HVR-H3 comprising the amino acid sequence of SEQ ID NO: 18. In another aspect, the invention provides an antibody comprising (a) HVR-H1 comprising the amino acid sequence of SEQ ID NO: 16; (b) HVR-H2 comprising the amino acid sequence of SEQ ID NO:17; ad (c) HVR-H3 comprising the amino acid sequence of SEQ ID NO: 18;
(iii) an anti-TGFβ2 antibody comprising at least one, at least two, or all three VL HVR sequences selected from (a) HVR-L1 comprising the amino acid sequence of SEQ ID NO: 19; (b) HVR-L2 comprising the amino acid sequence of SEQ ID NO: 20; and (c) HVR-L3 comprising the amino acid sequence of SEQ ID NO: 21. In one embodiment, the antibody comprises (a) HVR-L1 comprising the amino acid sequence of SEQ ID NO: 19; (b) HVR-L2 comprising the amino acid sequence of SEQ ID NO: 20; and (c) HVR-L3 comprising the amino acid sequence of SEQ ID NO: 21;
(iv) an anti-TGFβ2 antibody comprising HVR-H3 comprising the amino acid sequence of SEQ ID NO: 18 and HVR-L3 comprising the amino acid sequence of SEQ ID NO: 21. In a further embodiment, the anti-TGFβ2 antibody comprises HVR-H3 comprising the amino acid sequence of SEQ ID NO: 18, HVR-L3 comprising the amino acid sequence of SEQ ID NO: 21, and HVR-H2 comprising the amino acid sequence of SEQ ID NO: 17;
(v) an anti-TGFβ2 antibody comprising (a) a VH domain comprising at least one, at least two, or all three VH HVR sequences selected from (i) HVR-H1 comprising the amino acid sequence of SEQ ID NO: 16; (ii) HVR-H2 comprising the amino acid sequence of SEQ ID NO: 17; (iii) HVR-H3 comprising the amino acid sequence of SEQ ID NO: 18; and (b) a VL domain comprising at least one, at least two, or all three VL HVR sequences selected from (i) HVR-L1 comprising the amino acid sequence of SEQ ID NO: 19, (ii) HVR-L2 comprising the amino acid sequence of SEQ ID NO: 20, and (c) HVR-L3 comprising the amino acid sequence of SEQ ID NO: 21; and
(vi) an anti-TGFβ2 antibody comprising a) HVR-H1 comprising the amino acid sequence of SEQ ID NO: 16; (b) HVR-H2 comprising the amino acid sequence of SEQ ID NO:17; (c) HVR-H3 comprising the amino acid sequence of SEQ ID NO: 18; (d) HVR-L1 comprising the amino acid sequence of SEQ ID NO: 19; (e) HVR-L2 comprising the amino acid sequence of SEQ ID NO: 20; and (f) HVR-L3 comprising the amino acid sequence of SEQ ID NO: 21.

In any of the above embodiments for the methods of treating fibrosis, the anti-TGFβ2 antibody may be humanized. In one embodiment, the isoform-selective anti-TGFβ antibody comprises HVRs as in any of the above embodiments, and further comprises an acceptor human framework, e.g. a human immunoglobulin framework or a human consensus framework. In any of the above embodiments, a humanized anti-TGFβ2 antibody comprises one or more mutations in the VH framework selected from the group consisting of 37V or 37I, 48M or 48L, 49G or 49A, 67L, 71K and 78V, and 105P or 105R. In some embodiments, the anti-TGFβ2 antibody comprises a VH, wherein the VH comprises the amino acid sequence of SEQ ID NO: 25, wherein the VH comprises a set of framework mutations selected from the group consisting of: (i) 37V, 48M and 49G in FR2 and 105P in FR4 (h6F12.v1 and h6F12.v3); (ii) 37V and 48M in FR2, 67L, 71K and 78V in FR3 and 105P in FR4 (h6F12.v2 and h6F12.v4); (iii) 37I in FR2 (h6F12.v1.6); (iv) 48L in FR2 (h6F12.v1.7); (v) 49A in FR2 (h6F12.v1.8); (vi) 105R in FR4 (h6F12.v1.9); (vii) 37V, 48M and 49G in FR2 and 105P in FR4 (h6F12.v1 and h6F12.v3); (viii) 37V and 48M in FR2, 67L, 71K and 78V in FR3 and 105P in FR4 (6F12.v2 and h6F12.v4); (ix) 37I in FR2 (h6F12.v1.6); (x) 48L in FR2 (h6F12.v1.7); (xi): 49A in FR2 (h6F12.v1.8); and (xii) 105R in FR4 (h6F12.v1.9). In any of the above embodiments, a humanized anti-TGFβ2 antibody comprises one or more mutations in the VL framework selected from the group consisting of 43S or 43A, 66G, 69T, 71F, and 87Y. In some embodiments, the anti-TGFβ2 antibody comprises a VL, wherein the VL comprises the amino acid sequence of SEQ ID NO: 24, wherein the VL comprises a set of framework mutations selected from the group consisting of: (i) 43S in FR2 and 66E, 69P, 71Y and 87F in FR3 (h6F12.v1 and h6F12.v2); (ii) 43S in FR2 and 58V, 66E, 69P, 71Y and 87F in FR3 (h6F12.v3 and h6F12.v4); (iii) 43A in FR2 (h6F12.v1.1); (iv) 66G in FR3 (h6F12.v1.2); (v) 69T in FR3 (h6F12.v1.3); (vi) 71F in FR3 (h6F12.v1.4); and (vii) 87Y in FR3 (h6F12.v1.5).

In a further aspect, the invention provides a method for treating fibrosis, the method comprising administering to a subject having such fibrosis an effective amount of an isoform-selective anti-TGFβ antibody selected from the following:
an anti-TGFβ2 antibody comprising a heavy chain variable domain (VH) sequence having at least 90%, 91%, 92%, 93%, 94%, 95%, 96%, 97%, 98%, 99%, or 100% sequence identity to the amino acid sequence selected from the group consisting of SEQ ID NO: 25, 132, and 138-142; or the VH comprises one, two or three HVRs selected from: (a) HVR-H1 comprising the amino acid sequence of SEQ ID NO: 16; (b) HVR-H2 comprising the amino acid sequence of SEQ ID NO:17; (c) HVR-H3 comprising the amino acid sequence of SEQ ID NO: 18; and the antibody comprises a light chain variable domain (VL) having at least 90%, 91%, 92%, 93%, 94%, 95%, 96%, 97%, 98%, 99%, or 100% sequence identity to the amino acid sequence selected from the group consisting of SEQ ID NO: 24, 131, 133-137, 143, and 144, or the VL comprises one, two or three HVRs selected from (a) HVR-L1 comprising the amino acid sequence of SEQ ID NO: 19; (b) HVR-L2 comprising the amino acid sequence of SEQ ID NO: 20; and (c) HVR-L3 comprising the amino acid sequence of SEQ ID NO: 21;
an anti-TGFβ2 antibody comprising the VH/VL sequences (respectively) selected from the group consisting of SEQ ID NOs: 25/24 (rabbit 6F12), SEQ ID NOs: 132/131 (v1), SEQ ID NOs: 132/133 (v1.1), SEQ ID NOs: 132/134 (v1.2), SEQ ID NOs: 132/135 (v1.3), SEQ ID NOs: 132/136 (v1.4), SEQ ID NOs: 132/137 (v1.5), SEQ ID NOs: 138/131 (v1.6), SEQ ID NOs: 139/131 (v1.7), SEQ ID NOs: 140/131 (v1.8), SEQ ID NOs: 141/131 (v1.9), SEQ ID NOs: 142/131 (v2), SEQ ID NOs: 132/143 (v3), and SEQ ID NOs: 142/144 (v4); or
an anti-TGFβ2 antibody comprising a complete H chain amino acid sequence having at least 95% sequence identity to the amino acid sequence selected from the group consisting of SEQ ID NOs: 31, 146, and 152-156 and/or a complete L chain amino acid sequence having an amino acid sequence selected from the group consisting of SEQ ID NOs: 30, 145, 147-151, 157, and 158;
an anti-TGFβ2 antibody comprising a complete H/L chain pair, the complete H/L chain pair (respectively) comprising amino acid sequences selected from the group consisting of SEQ ID NOs: 31/30 (rabbit 6F12), SEQ ID NOs: 146/145 (v1), SEQ ID NOs: 146/147 (v1.1), SEQ ID NOs: 146/148 (v1.2), SEQ ID NOs: 146/149 (v1.3), SEQ ID NOs: 146/150 (v1.4), SEQ ID NOs: 146/151 (v1.5), SEQ ID NOs: 152/145 (v1.6), SEQ ID NOs: 153/145 (v1.7), SEQ ID NOs: 154/145 (v1.8), SEQ ID NOs: 155/145 (v1.9), SEQ ID NOs: 156/145 (v2), SEQ ID NOs: 146/157 (v3), and SEQ ID NOs: 156/158 (v4); or
an antibody that binds to the same epitope as an anti-TGFβ2 antibody comprising a) HVR-H1 comprising the amino acid sequence of SEQ ID NO: 16; (b) HVR-H2 comprising the amino acid sequence of SEQ ID NO:17; (c) HVR-H3 comprising the amino acid sequence of SEQ ID NO: 18; (d) HVR-L1 comprising the amino acid sequence of SEQ ID NO: 19; (e) HVR-L2 comprising the amino acid sequence of SEQ ID NO: 20; and (f) HVR-L3 comprising the amino acid sequence of SEQ ID NO: 21.

### Anti-TGFβ2/3 Antibodies

In some aspects of the above methods for treating a TGFβ-related disorder, such as but not limited to fibrosis (e.g., fibrosis of the lung, liver, pancreas, heart, kidney, eye, and/or skin) are provided, the method comprises administering to a subject having such TGFβ-related disorder (e.g.) fibrosis an effective amount of an isoform-selective anti-TGFβ2/3 antibody of the invention that selectively neutralizes TGFβ2 and TGFβ3 and has one or more of the following properties:
(a) selectivity of the anti-TGFβ2/3 antibody for TGFβ2 and TGFβ3 over human TGFβ1, with respect to selective neutralization, is achieved by direct contact of the antibody's antigen binding domain with amino acid residue E373 of TGFβ2 or TGFβ3 (human TGFβ2 numbering);
(b) the anti-TGFβ2/3 antibody neutralizes TGFβ2 and/or TGFβ3 via an allosteric mechanism;
(c) the anti-TGFβ2/3 antibody induces a conformational change in TGFβ2 homodimer;
(d) the anti-TGFβ2/3 antibody induces a conformational change in TGFβ2 homodimer, wherein the conformational change comprises the two monomers pinching together by several degrees;
(e) the anti-TGFβ2/3 antibody is a divalent antibody or a monovalent antibody; (f) the anti-TGFβ2/3 antibody comprises (i) heavy chain CDRs comprising CDR-H1, -H2 and -H3, wherein CDR-H1 has the amino acid sequence of SEQ ID NO: 10, CDR-H2 has the amino acid sequence of SEQ ID NO: 11, and CDR-H3 has the amino acid sequence of SEQ ID NO: 12; and (ii) light chain CDRs comprising CDR-L1, -L2 and -L3, wherein CDR-L1 has the amino acid sequence of SEQ ID NO: 13; CDR-L2 has the amino acid sequence of SEQ ID NO: 14, and CDR-L3 has the amino acid sequence of SEQ ID NO: 15;
(g) the anti-TGFβ2/3 antibody binds to TGFβ2 homodimer, the TGFβ2 homodimer having a first and a second TGFβ2 monomer, and wherein the anti-TGFβ2/3 antibody comprises an antigen binding domain that directly contacts (i) amino acid residues V313, Q314, D315, R320, L322, Y323, R328, D329, F345, and A347 of the first TGFβ2 monomer, and (ii) amino acid residues N368, T369, I370, N371, P372, E373, A374, S375, A376, and S377 of the second TGFβ2 monomer (human TGFβ2 numbering);
(h) the anti-TGFβ2/3 antibody as in (g), wherein the antigen binding domain is within 5 angstroms of the TGFβ2 and/or TGFβ3 amino acid residues;
(i) wherein the anti-TGFβ2/3 antibody binds to a substantially similar epitope as in (g) in TGFβ3; and
(j) the anti-TGFβ2/3 antibody does not neutralize TGFβ2 and/or TGFβ3 in single arm form.

In a further aspect, the invention provides a method for treating fibrosis, the method comprising administering to a subject having such fibrosis an effective amount of an anti-TGFβ2/3 antibody that has one or more of the following properties:
selectively neutralizes TGFβ2 and TGFβ3; binds to TGFβ2 and/or TGFβ3 with a K_{D} of less than 10 pM and/or has a cell-based IC₅₀ less than 250 pM;
binds to TGFβ2 and/or TGFβ3 with a K_{D} of less than about 10 pM, about 9 pM, about 8 pM, about 7 pM, about 6 pM, about 5 pM, about 4 pM, about 3 pM, about 2 pM, or about 1 pM;
has a cell-based IC₅₀ for inhibition of TGFβ2 of about 250 pM, about 200 pM, about 150 pM, about 100 pM, about 75 pM, or about 50 pM;
has a cell-based IC₅₀ for inhibition of TGFβ3 of about 250 pM, about 200 pM, about 150 pM, about 100 pM, about 75 pM, about 50 pM, about 40 pM, about 30 pM or less than about 30 pM; or
has a cell-based IC₅₀ for inhibition of TGFβ2 of about 250 pM and a cell-based IC₅₀ for inhibition (neutralization) of TGFβ3 of about 30 pM.

In a further aspect, the invention provides a method for treating fibrosis, the method comprising administering to a subject having such fibrosis an effective amount of an anti-TGFβ2/3 antibody selected from the following:
an antibody comprising at least one, two, three, four, five, or six HVRs selected from (a) HVR-H1 comprising the amino acid sequence of SEQ ID NO: 10; (b) HVR-H2 comprising the amino acid anti-TGFβ2/3 antibody sequence of SEQ ID NO: 11; (c) HVR-H3 comprising the amino acid sequence of SEQ ID NO: 12; (d) HVR-L1 comprising the amino acid sequence of SEQ ID NO: 13; (e) HVR-L2 comprising the amino acid sequence of SEQ ID NO: 14; and (f) HVR-L3 comprising the amino acid sequence of SEQ ID NO: 15;
an antibody comprising at least one, at least two, or all three VH HVR sequences selected from (a) HVR-H1 comprising the amino acid sequence of SEQ ID NO: 10; (b) HVR-H2 comprising the amino acid anti-TGFβ2/3 antibody sequence of SEQ ID NO: 11; (c) HVR-H3 comprising the amino acid sequence of SEQ ID NO: 12, and at least one, at least two, or all three VL HVR sequences selected from (a) HVR-L1 comprising the amino acid sequence of SEQ ID NO: 13; (b) HVR-L2 comprising the amino acid sequence of SEQ ID NO: 14; and (c) HVR-L3 comprising the amino acid sequence of SEQ ID NO: 15;
an antibody comprising (a) HVR-H1 comprising the amino acid sequence of SEQ ID NO: 10; (b) HVR-H2 comprising the amino acid sequence of SEQ ID NO: 11; (c) HVR-H3 comprising the amino acid sequence of SEQ ID NO: 12; (d) HVR-L1 comprising the amino acid sequence of SEQ ID NO: 13; (e) HVR-L2 comprising the amino acid sequence of SEQ ID NO: 14; and (f) HVR-L3 comprising an amino acid sequence selected from SEQ ID NO: 15.

In any of the above embodiments for treating fibrosis, the anti-TGFβ2/3 antibody may be humanized. In one embodiment, an anti-TGFβ2/3 antibody comprises HVRs as in any of the above embodiments, and further comprises an acceptor human framework, e.g. a human immunoglobulin framework or a human consensus framework. In some embodiments, the anti-TGFβ2/3 antibody comprises a VH, wherein the VH comprises the amino acid sequence of SEQ ID NO: 27, wherein the VH comprises a set of framework modifications selected from the group consisting of: (i) 2Q and 24V in FR1, 48I and 49G in FR2, 71K, 73S, 78V and 91F in FR3 and 105P in FR4 (h4A11.v, h4A11.v2, h4A11.v5, h4A11.v6); (ii) 2Q in FR1, 37V in FR2, 67F, 71K, 73S, 78V and 91F in FR3 and 105P in FR4 (h4A11.v3, h4A11.v4, 4A11.v7, h4A11.v8); (iii) delete 1E in FR1 (h4A11.v7.1); (iv) delete 75K and 76N in FR3 (h4A11.v7.2); (v) delete 1E in FR1 and 75K76N in FR3 (h4A11.v7.3); (vi) 2V in FR1 (h4A11.v7.8); (vi) 37I in FR2 (h4A11.v7.9); (vii) 67V in FR3 (h4A11.v7.10); (viii) 71V in FR3 (h4A11.v7.11); (ix) 73T in FR3 (h4A11.v7.12); (x) 78F in FR3 (h4A11.v7.13); (xi) 91Y in FR3 (h4A11.v7.14); (xii) 105Q in FR4 (h4A11.v7.15); (xiii) 2V in FR1, 37I in FR2, 67V, 73T, 78F in FR3, 105Q in FR4 ((h4A11.v7.16); (xiv) 2V in FR1, 37I in FR2, 67V, 73T, 91Y in FR3, 105Q in FR4 (h4A11.v7.17); (xv) 2V in FR1, 37I in FR2, 67V, 73T in FR3, 105Q in FR4 (h4A11.v7.18); and (xvi) 2V in FR1, 37I in FR2, 67V, 73T, deletion of 75K and 76N in FR3, 105Q in FR4 (h4A11.v7.19). In another embodiment, an anti-TGFβ2/3 antibody comprises HVRs as in any of the above embodiments, and further comprises a VL comprising FR modifications selected from the group consisting of: 2A or 2I, 4L, 36F or 36Y, 43P or 43A, and 58V or 58I, relative to the VL amino acid sequence of SEQ ID NO: 26. In some embodiments, the anti-TGFβ2/3 antibody comprises a VL, wherein the VL comprises the amino acid sequence of SEQ ID NO: 26, wherein the VL comprises a set of framework modifications selected from the group consisting of: (i) 2A and 4L in FR1 and 36F in FR2 (h4A11.v1 and h4A11.v3); (ii) 2A and 4L in FR1 and 36F and 43P in FR2 (h4A11.v2 and h4A11.v4); (iii) 2A in FR1, 36F and 43P in FR2 and 58V in FR3 (h4A11.v5 and h4A11.v7); (iv) 2A and 4L in FR1 and 36F in FR2 (h4A11.v6 and h4A11.v8); (v) 2I in FR1 (h4A11.v7.4); (vi) 36Y in FR2 (h4A11.v7.5); (vii) 43A in FR2 (h4A11.v7.6); (viii) 58I in FR3 (h4A11.v7.7); and (ix) 2I in FR1, 43A in FR2, 58I in FR3 (h4A11.v7.16-19).

In a further aspect, the invention provides a method for treating fibrosis, the method comprising administering to a subject having such fibrosis an effective amount of an anti-TGFβ2/3 antibody selected from the following:
an antibody comprising a heavy chain variable domain (VH) sequence having at least 90%, 91%, 92%, 93%, 94%, 95%, 96%, 97%, 98%, 99%, or 100% sequence identity to the amino acid sequence selected from the group consisting of SEQ ID NOs: 27, 81, 83, 86-88, 93-100, and 102-105, and a light chain variable domain (VL) having at least 90%, 91%, 92%, 93%, 94%, 95%, 96%, 97%, 98%, 99%, or 100% sequence identity to the amino acid sequence selected from the group consisting of SEQ ID NOs: 26, 80, 82, 84, 85, 89-92, and 101;
an antibody comprising the VH and VL sequences (respectively) selected from the group consisting of SEQ ID NOs: 27/26 (rabbit 4A11), SEQ ID NOs: 81/80 (v1), SEQ ID NOs: 81/82 (v2), SEQ ID NOs: 83/80 (v3), SEQ ID NOs: 83/82 (v4), SEQ ID NOs: 81/84 (v5), SEQ ID NOs: 81/85 (v6), SEQ ID NOs: 83/84 (v7), SEQ ID NOs: 86/84 (v7/1), SEQ ID NOs: 87/84 (v7.2), SEQ ID NOs: 88/84 (v7.3), SEQ ID NOs: 83/89 (v7.4), SEQ ID NOs: 83/90 (v7.5), SEQ ID NOs: 83/91 (v7.6), SEQ ID NOs: 83/92 (v7.7), SEQ ID NOs: 93/84 (v7.8), SEQ ID NOs: 94/84 (v7.9), SEQ ID NOs: 95/84 (v7.10), SEQ ID NOs: 96/84 (v7.11), SEQ ID NOs: 97/84 (v7.12), SEQ ID NOs: 98/84 (v7.13), SEQ ID NOs: 99/84 (v7.14), SEQ ID NOs: 100/84 (v7.15), SEQ ID NOs: 102/101 (v7.16), SEQ ID NOs: 103/101 (v7.17), SEQ ID NOs: 104/101 (v7.18), SEQ ID NOs: 105/101 (v7.19), SEQ ID NOs: 83/85 (v8);
an antibody comprising a complete H chain amino acid sequence having at least 95% sequence identity to the amino acid sequence selected from the group consisting of SEQ ID NOs: 33, 107, 109, 112-114, and 119-130 and/or a complete L chain amino acid sequence having an amino acid sequence selected from the group consisting of SEQ ID NOs: 32, 106, 108, 110, 111, 115-118, and 186;
an antibody comprising a complete H/L chain pair, the complete H/L chain pair (respectively) comprising amino acid sequences selected from the group consisting of SEQ ID NOs: 32/33 (rabbit 4A11), SEQ ID NOs: 107/106 (vi), SEQ ID NOs: 107/108 (v2), SEQ ID NOs: 109/106 (v3), SEQ ID NOs: 109/108 (v4), SEQ ID NOs: 107/110 (v5), SEQ ID NOs: 107/111 (v6), SEQ ID NOs: 109/110 (v7), SEQ ID NOs: 112/110 (v7.1), SEQ ID NOs: 113/110 (v7.2), SEQ ID NOs: 114/110 (v7.3), SEQ ID NOs: 114/115 (v7.4), SEQ ID NOs: 114/116 (v7.5), SEQ ID NOs: 114/117 (v7.6), SEQ ID NOs: 114/118 (v7.7), SEQ ID NOs: 119/110 (v7.8), SEQ ID NOs:120/110 (v7.9), SEQ ID NOs: 121/110 (v7.10), SEQ ID NOs: 122/110 (v7.11), SEQ ID NOs: 123/110 (v7.12), SEQ ID NOs: 124/110 (v7.13), SEQ ID NOs: 125/110 (v7.14), SEQ ID NOs: 126/110 (v7.15), SEQ ID NOs: 127/186 (v7.16), SEQ ID NOs: 128/186 (v7.17), SEQ ID NOs: 129/186 (v7.18), SEQ ID NOs: 130/186 (v7.19), and SEQ ID NOs: 114/111 (v8);
an antibody that binds to the same epitope as an anti-TGFβ2/3 antibody comprising (a) HVR-H1 comprising the amino acid sequence of SEQ ID NO: 10; (b) HVR-H2 comprising the amino acid sequence of SEQ ID NO: 11; (c) HVR-H3 comprising the amino acid sequence of SEQ ID NO: 12; (d) HVR-L1 comprising the amino acid sequence of SEQ ID NO: 13; (e) HVR-L2 comprising the amino acid sequence of SEQ ID NO: 14; and (f) HVR-L3 comprising an amino acid sequence selected from SEQ ID NO: 15;
an antibody that binds to an epitope spanning a TGFβ2 homodimer, the TGFβ2 homodimer having a first and a second TGFβ2 monomer, wherein the anti-TGFβ2/3 antibody comprises an antigen-binding domain that directly contacts amino acid residues V313, Q314, D315, R320, L322, Y323, R328, D329, F345, and A347 of the first TGFβ2 monomer, and amino acid residues N368, T369, I370, N371, P372, E373, A374, S375, A376, and S377 of the second TGFβ2 monomer, and wherein, in some embodiments, the anti-TGFβ2/3 antibody binds to the same epitope in TGFβ3.

### Anti-TGFβ3 Antibodies

In some aspects of the above methods for treating a TGFβ-related disorder, such as but not limited to fibrosis (e.g., fibrosis of the lung, liver, pancreas, heart, kidney, eye, and/or skin), are provided, the method comprises administering to a subject having such TGFβ-related disorder an effective amount of an anti-TGFβ3 antibody that selectively neutralizes TGFβ3 having one or more of the following properties:
(a) the anti-TGFβ3 antibody binds to the beta6/beta7 hairpin region of TGFβ3;
(b) binding of the anti-TGFβ3 antibody sterically blocks the ability of TGFBR2, but not TGFBR1, to bind TGFβ3;
(c) binding of the anti-TGFβ3 antibody to TGFβ3 blocks TGFBR2 binding and inhibits the TGFBR1/TGFBR2 signaling receptors from binding to TGFβ3;
(d) the anti-TGFβ3 antibody binds amino acid residue R394 in the beta6/beta7 hairpin region of human TGFβ3;
(e) the anti-TGFβ3 antibody binds amino acid residue R394 in the beta6/beta7 hairpin region of human TGFβ3 and residue R394 of TGFβ3 makes an ionic salt bridge with the anti-TGFβ3 antibody in the heavy chain CDR2;
(f) isoform selectivity of the anti-TGFβ3 antibody for TGFβ3 over TGFβ1 is achieved by direct contact by the antigen binding domain of the anti-TGFβ3 antibody with amino acid residues T387, L389, and T395 of TGFβ3 (human TGFβ3 numbering);
(g) the isoform selectivity of the anti-TGFβ3 antibody for TGFβ3 over TGFβ2 is achieved by direct contact by the antigen binding domain of the anti-TGFβ3 antibody with amino acid residues R325, R394, and V398 of TGFβ3 (human TGFβ3 numbering);
(h) the anti-TGFβ3 antibody has reduced toxicity relative to the pan-TGFβ antibody 1D11;
(i) the anti-TGFβ3 antibody has reduced toxicity in rodents or cynomolgus monkeys relative to the pan-TGFβ antibody 1D11;
(j) the anti-TGFβ3 antibody has reduced toxicity relative to the pan-TGFβ small molecule inhibitor galunisertib;
(k) the anti-TGFβ3 antibody has reduced toxicity in rodents relative to the pan-TGFβ small molecule inhibitor galunisertib;
(l) the anti-TGFβ3 antibody has reduced toxicity relative to the anti-TGFβ1 antibody CAT-192;
(m) the anti-TGFβ3 antibody has reduced toxicity relative to an isoform selective anti-TGFβ2 antibody and/or anti-TGFβ2/3 antibody;
(n) the anti-TGFβ3 antibody comprises: (i) heavy chain CDRs comprising CDR-H1, -H2 and -H3, wherein CDR-H1 has the amino acid sequence of SEQ ID NO: 4, CDR-H2 has the amino acid sequence of one of SEQ ID NOs: 5, 34, 35, and 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9;
(o) the anti-TGFβ3 antibody comprises an antigen binding domain that directly contacts amino acid residues R325, K331, W332, H334, E335, T387, I388, L389, Y391, V392, G393, R394, P396, K397, and V398 on human TGFβ3; and
(p) the anti-TGFβ3 antibody as in (o), wherein the antigen binding domain is within 15-8, 8, 8-5, 7-5, 6-5, or 5 angstroms of the TGFβ3 amino acid residues.

In a further aspect, the invention provides a method for treating fibrosis, the method comprising administering to a subject having such fibrosis an effective amount of an isoform-selective anti-TGFβ antibody having one or more of the following properties:
an antibody that selectively neutralizes TGFβ3;
an antibody that binds to TGFβ3 with a K_{D} of less than 10 pM and/or has a cell-based IC₅₀ less than 250 pM;
an antibody that binds to TGFβ3 with a K_{D} of less than about 5 pM, about 4 pM, about 3 pM, or less than about 2 pM;
an antibody that has a cell-based IC₅₀ less of than about 200 pM, about 150 pM, about 100 pM, about 75 pM, or about 50 pM;
an antibody provided herein that has a cell-based IC₅₀ for inhibition (neutralization) of TGFβ3 of less than about 20 pM;
an antibody that selectively neutralizes TGFβ3 and has reduced toxicity in mice relative to the pan-TGFβ antibody 1D11;
an antibody that selectively neutralizes TGFβ3 and has an improved safety profile.

In a further aspect, the invention provides a method for treating fibrosis, the method comprising administering to a subject having such fibrosis an effective amount of an anti-TGFβ3 antibody selected from the following:
an antibody comprising at least one, two, three, four, five, or six HVRs selected from (a) HVR-H1 comprising the amino acid sequence of SEQ ID NO: 4; (b) HVR-H2 comprising the amino acid sequence selected from SEQ ID NOs: 5, 34, 35, and 159; (c) HVR-H3 comprising the amino acid sequence of SEQ ID NO: 6; (d) HVR-L1 comprising the amino acid sequence of SEQ ID NO: 7; (e) HVR-L2 comprising the amino acid sequence of SEQ ID NO: 8; and (f) HVR-L3 comprising the amino acid sequence of SEQ ID NO: 9;
an antibody comprising (a) HVR-H1 comprising the amino acid sequence of SEQ ID NO: 4; (b) HVR-H2 comprising the amino acid sequence of SEQ ID NO: 5; (c) HVR-H3 comprising the amino acid sequence of SEQ ID NO: 6; (d) HVR-L1 comprising the amino acid sequence of SEQ ID NO: 7; (e) HVR-L2 comprising the amino acid sequence of SEQ ID NO: 8; and (f) HVR-L3 comprising an amino acid sequence selected from SEQ ID NO: 9;
an antibody comprising (a) HVR-H1 comprising the amino acid sequence of SEQ ID NO: 4; (b) HVR-H2 comprising the amino acid sequence of SEQ ID NO: 34; (c) HVR-H3 comprising the amino acid sequence of SEQ ID NO: 6; (d) HVR-L1 comprising the amino acid sequence of SEQ ID NO: 7; (e) HVR-L2 comprising the amino acid sequence of SEQ ID NO: 8; and (f) HVR-L3 comprising an amino acid sequence selected from SEQ ID NO: 9;
an antibody comprising (a) HVR-H1 comprising the amino acid sequence of SEQ ID NO: 4; (b) HVR-H2 comprising the amino acid sequence of SEQ ID NO: 35; (c) HVR-H3 comprising the amino acid sequence of SEQ ID NO: 6; (d) HVR-L1 comprising the amino acid sequence of SEQ ID NO: 7; (e) HVR-L2 comprising the amino acid sequence of SEQ ID NO: 8; and (f) HVR-L3 comprising an amino acid sequence selected from SEQ ID NO: 9; and
an antibody comprising (a) HVR-H1 comprising the amino acid sequence of SEQ ID NO: 4; (b) HVR-H2 comprising the amino acid sequence of SEQ ID NO: 159; (c) HVR-H3 comprising the amino acid sequence of SEQ ID NO: 6; (d) HVR-L1 comprising the amino acid sequence of SEQ ID NO: 7; (e) HVR-L2 comprising the amino acid sequence of SEQ ID NO: 8; and (f) HVR-L3 comprising an amino acid sequence selected from SEQ ID NO: 9.

In certain embodiments, any one or more amino acids of an anti-TGFβ3 antibody as provided above are substituted at the following HVR positions:
in HVR-H2 (SEQ ID NO: 5): at position N54 (e.g., N54S, N54Q) or T56 (e.g., T56A). In certain embodiments, the substitutions are conservative substitutions, as provided herein.

In any of the above embodiments of methods for treating fibrosis, an anti-TGFβ3 antibody may be humanized. In one embodiment, an anti-TGFβ3 antibody comprises HVRs as in any of the above embodiments, and further comprises an acceptor human framework, e.g. a human immunoglobulin framework or a human consensus framework. In another embodiment, an anti-TGFβ3 antibody comprises HVRs as in any of the above embodiments, and further comprises a VH comprising FR modifications selected from the group consisting of 47L or 47W; 49A, 49S, or 49G; 73D or 73N; and 76N, 78D or 78L, 78A, or 78V, relative to the VH having amino acid sequence of SEQ ID NO: 23. In some embodiments, the anti-TGFβ3 antibody comprises a VH, wherein the VH comprises the amino acid sequence of SEQ ID NO: 23, wherein the VH comprises a set of framework modifications selected from the group consisting of: (i) 47L, 49A in FR2, 78V in FR3 (h2A10.v1); (ii) 47L, 49A in FR2, 73D, 76S, 78V in FR3 (h2A10.v2); (iii) 47W in FR2 (h2A10.v1.5); (iv): 49G in FR2 (h2A10.v1.6); (v) 78A in FR3 (h2A10.v1.7); (vi) 47W in FR2 (h2A10.v2.5); (vii) 49S in FR2 (h2A10.v2.6); (viii) 73N in FR3 (h2A10.v2.7); (ix) 76N in FR3 (h2A10.v2.8); (x) 78L in FR3 (h2A10.v2.9); and (xi) 49S in FR2, 76N, 78L in FR3 (h2A10.v3 and h2A10.v4). In another embodiment, an anti-TGFβ3 antibody comprises HVRs as in any of the above embodiments, and further comprises a VL comprising FR modifications selected from the group consisting of: 4L or 4M, 38H or 38Q, 43A or 43Q, and 58V, relative to the VL having amino acid sequence of SEQ ID NO; 22. In another embodiment, the antibody comprises the VL of SEQ ID NO: 22, wherein the VL comprises a set of framework modifications selected from the group consisting of: (i) 4L in FR1, 38H and 43Q in FR2, 58I in FR3 (h2A10.v1 and h2A10.v2); (ii) 4M in FR1 (h2A10.v1.1 and h2A10.v2.1); (iii) 38Q in FR2 (h2A10.v1.2 and h2A10.v2.2); (iv) 43A in FR2 (h2A10.v1.3 and h2A10.v2.3); (v) 58V in FR3 (h2A10.v1.4 and h2A10.v2.4); (vi) 38Q, 43A in FR2, 58V in FR3 (h2A10.v3 and h2A10.v4); (vii) 58V in FR3 (h2A10.v1.4 and h2A10.v2.4); and (vi) 38Q, 43A in FR2, 58V in FR3 (h2A10.v3 and h2A10.v4).

In a further aspect, the invention provides a method for treating fibrosis, the method comprising administering to a subject having such fibrosis an effective amount of an anti-TGFβ3 antibody selected from the following:
an antibody comprising a heavy chain variable domain (VH) sequence having at least 90%, 91%, 92%, 93%, 94%, 95%, 96%, 97%, 98%, 99%, or 100% sequence identity to the amino acid sequence selected from the group consisting of SEQ ID NOs: 23, 37, and 42-50, and a light chain variable domain (VL) having at least 90%, 91%, 92%, 93%, 94%, 95%, 96%, 97%, 98%, 99%, or 100% sequence identity to the amino acid sequence selected from the group consisting of SEQ ID NOs: 22, 36, and 38-41;
an antibody comprising VH/VL sequences (respectively) selected from the group consisting of SEQ ID NOs: 23/22 (rat 2A10), SEQ ID NOs: 37/36 (v1), SEQ ID NOs: 37/38 (v1.1), SEQ ID NOs: 37/39 (v1.2), SEQ ID NOs: 37/40 (v1.3), SEQ ID NOs: 37/41 (v1.4), SEQ ID NOs: 42/36 (v1.5), SEQ ID NOs: 43/36 (v1.6), SEQ ID NOs:44/36 (v1.7), SEQ ID NOs: 45/36 (v2), SEQ ID NOs: 45/38 (v2.1), SEQ ID NOs: 45/39 (v2.2), SEQ ID NOs: 45/40 (v2.3), SEQ ID NOs: 45/41 (v2.4), SEQ ID NOs: 46/36 (v2.5), SEQ ID NOs: 47/36 (v2.6), SEQ ID NOs: 48/36 (v2.7), SEQ ID NOs: 49/36 (v2.8), and SEQ ID NOs: 50/36 (v2.9);
an antibody comprising a heavy chain variable domain (VH) sequence having at least 90%, 91%, 92%, 93%, 94%, 95%, 96%, 97%, 98%, 99%, or 100% sequence identity to the amino acid sequence selected from the group consisting of SEQ ID NOs: 23, 37, 42-50, 55, and 57, and a light chain variable domain (VL) having at least 90%, 91%, 92%, 93%, 94%, 95%, 96%, 97%, 98%, 99%, or 100% sequence identity to the amino acid sequence selected from the group consisting of SEQ ID NOs: 22, 36, 38-41, 54, and 56;
an antibody comprising VH/VL sequences (respectively) selected from the group consisting of SEQ ID NOs: 23/22 (rat 2A10), SEQ ID NOs: 37/36 (v1), SEQ ID NOs: 37/38 (v1.1), SEQ ID NOs: 37/39 (v1.2), SEQ ID NOs: 37/40 (v1.3), SEQ ID NOs: 37/41 (v1.4), SEQ ID NOs: 42/36 (v1.5), SEQ ID NOs: 43/36 (v1.6), SEQ ID NOs:44/36 (v1.7), SEQ ID NOs: 45/36 (v2), SEQ ID NOs: 45/38 (v2.1), SEQ ID NOs: 45/39 (v2.2), SEQ ID NOs: 45/40 (v2.3), SEQ ID NOs: 45/41 (v2.4), SEQ ID NOs: 46/36 (v2.5), SEQ ID NOs: 47/36 (v2.6), SEQ ID NOs: 48/36 (v2.7), SEQ ID NOs: 49/36 (v2.8), SEQ ID NOs: 50/36 (v2.9), SEQ ID NOs: 55/54 (v3), and SEQ ID NOs: 57/56 (v4);
an antibody comprises a complete H chain amino acid sequence having at least 95% sequence identity to the amino acid sequence selected from the group consisting of SEQ ID NOs: 29, 59, 64, and 65-72 and/or a complete L chain amino acid sequence having an amino acid sequence selected from the group consisting of SEQ ID NOs: 28, 58, and 60-63;
an antibody comprising a complete H/L chain pair, the complete H/L chain pair (respectively) comprising amino acid sequences selected from the group consisting of SEQ ID NOs: 29/28 (rat 2A10), SEQ ID NOs: 59/58 (vi), SEQ ID NOs: 59/60 (v1.1), SEQ ID NOs: 59/61 (v1.2), SEQ ID NOs: 59/62 (v1.3), SEQ ID NOs: 59/63 (v1.4), SEQ ID NOs: 64/58 (v1.5), SEQ ID NOs: 65/58 (v1.6), SEQ ID NOs: 66/58 (v1.7), SEQ ID NOs: 67/58 (v2), SEQ ID NOs: 67/60 (v2.1), SEQ ID NOs: 67/61 (v2.2), SEQ ID NOs: 67/62 (v2.3), SEQ ID NOs: 67/63 (v2.4), SEQ ID NOs: 68/58 (v2.5), SEQ ID NOs: 69/58 (v2.6), SEQ ID NOs: 70/58 (v2.7), SEQ ID NOs: 71/58 (v2.8), SEQ ID NOs: 72/58 (v2.9);
an antibody comprises a complete H chain amino acid sequence having at least 95% sequence identity to the amino acid sequence selected from the group consisting of SEQ ID NOs: 29, 59, 64, 65-72, 77, and 79 and/or a complete L chain amino acid sequence having an amino acid sequence selected from the group consisting of SEQ ID NOs: 28, 58, 60-63, 76, and 78;
an antibody comprising a complete H/L chain pair, the complete H/L chain pair (respectively) comprising amino acid sequences selected from the group consisting of SEQ ID NOs: 29/28 (rat 2A10), SEQ ID NOs: 59/58 (vi), SEQ ID NOs: 59/60 (v1.1), SEQ ID NOs: 59/61 (v1.2), SEQ ID NOs: 59/62 (v1.3), SEQ ID NOs: 59/63 (v1.4), SEQ ID NOs: 64/58 (v1.5), SEQ ID NOs: 65/58 (v1.6), SEQ ID NOs: 66/58 (v1.7), SEQ ID NOs: 67/58 (v2), SEQ ID NOs: 67/60 (v2.1), SEQ ID NOs: 67/61 (v2.2), SEQ ID NOs: 67/62 (v2.3), SEQ ID NOs: 67/63 (v2.4), SEQ ID NOs: 68/58 (v2.5), SEQ ID NOs: 69/58 (v2.6), SEQ ID NOs: 70/58 (v2.7), SEQ ID NOs: 71/58 (v2.8), SEQ ID NOs: 72/58 (v2.9), SEQ ID NOs: 77/76 (v3), and SEQ ID NOs: 79/78;
an antibody comprising a VH having at least 90%, 91%, 92%, 93%, 94%, 95%, 96%, 97%, 98%, 99%, or 100% sequence identity to the amino acid sequence of SEQ ID NO: 52 and a VL amino acid sequence having at least 90%, 91%, 92%, 93%, 94%, 95%, 96%, 97%, 98%, 99%, or 100% sequence identity to an amino acid sequence selected from the group consisting of SEQ ID NOs: 22, 36, and 38-41;
an antibody comprising VH/VL sequences (respectively) comprising the amino acid sequences of SEQ ID NOs: 52/36 (h2A10.v2.N54Q);
an antibody comprising a complete H chain having at least 90%, 91%, 92%, 93%, 94%, 95%, 96%, 97%, 98%, 99%, or 100% sequence identity to the amino acid sequence of SEQ ID NO: 74 and a complete L chain amino acid sequence having at least 90%, 91%, 92%, 93%, 94%, 95%, 96%, 97%, 98%, 99%, or 100% sequence identity to an amino acid sequence selected from the group consisting of SEQ ID NOs: 28, 58, and 60-63;
an antibody comprising complete H/L chain sequences (respectively) comprising the amino acid sequences of SEQ ID NOs: 74/58 (h2A10.v2.N54Q);
an antibody comprising a VH having at least 90%, 91%, 92%, 93%, 94%, 95%, 96%, 97% 98%, 99%, or 100% sequence identity to the amino acid sequence of SEQ ID NO: 51 or 55 and a VL amino acid sequence having at least 90%, 91%, 92%, 93%, 94%, 95%, 96%, 97%, 98%, 99%, or 100% sequence identity to an amino acid sequence selected from the group consisting of SEQ ID NOs: 22, 36, and 38-41;
an antibody comprising a VH having at least 90%, 91%, 92%, 93%, 94%, 95%, 96%, 97% 98%, 99%, or 100% sequence identity to the amino acid sequence of SEQ ID NO: 51 or 55 and a VL amino acid sequence having at least 90%, 91%, 92%, 93%, 94%, 95%, 96%, 97%, 98%, 99%, or 100% sequence identity to an amino acid sequence selected from the group consisting of SEQ ID NOs: 22, 36, 38-41, and 54;
an antibody comprising VH/VL sequences (respectively) comprising the amino acid sequences of SEQ ID NO: 51/36 (h2A10.v2.N54S) or SEQ ID NO: 55/54 (h2A10.v3);
an antibody comprising a complete H chain having at least 95% sequence identity to the amino acid sequence of SEQ ID NO: 73 or 77 and/or the anti-TGFβ3 antibody comprises a complete L chain amino acid sequence having at least 90%, 91%, 92%, 93%, 94%, 95%, 96%, 97%, 98%, 99%, or 100% sequence identity to an amino acid sequence selected from the group consisting of SEQ ID NOs: 28, 58, and 60-63;
an antibody comprising a complete H chain having at least 95% sequence identity to the amino acid sequence of SEQ ID NO: 73 or 77 and/or the anti-TGFβ3 antibody comprises a complete L chain amino acid sequence having at least 90%, 91%, 92%, 93%, 94%, 95%, 96%, 97%, 98%, 99%, or 100% sequence identity to an amino acid sequence selected from the group consisting of SEQ ID NOs: 28, 58, 60-63, and 76;
an antibody comprising complete H/L chain sequences (respectively) comprising the amino acid sequences of SEQ ID NOs: 73/58 (h2A10.v2.N54S) or SEQ ID NOs: 77/76 (h2A10.v3);
an antibody comprising a VH having at least 90%, 91%, 92%, 93%, 94%, 95%, 96%, 97%, 98%, 99%, or 100% sequence identity to the amino acid sequence of SEQ ID NO: 53 or 57, and a VL amino acid sequence having at least 90%, 91%, 92%, 93%, 94%, 95%, 96%, 97%, 98%, 99%, or 100% sequence identity to an amino acid sequence selected from the group consisting of SEQ ID NOs: 22, 36, and 38-41;
an antibody comprising a VH having at least 90%, 91%, 92%, 93%, 94%, 95%, 96%, 97%, 98%, 99%, or 100% sequence identity to the amino acid sequence of SEQ ID NO: 53 or 57, and a VL amino acid sequence having at least 90%, 91%, 92%, 93%, 94%, 95%, 96%, 97%, 98%, 99%, or 100% sequence identity to an amino acid sequence selected from the group consisting of SEQ ID NOs: 22, 36, 38-41, and 56;
an antibody comprising VH/VL sequences (respectively) comprising the amino acid sequences of SEQ ID NOs: 53/36 (h2A10.v2.T56A) or SEQ ID NOs: 57/56 (h2A10.v4);
an antibody comprising a complete H chain having at least 95% sequence identity to the amino acid sequence of SEQ ID NO: 74 or 79 and/or a complete L chain amino acid sequence having at least 90%, 91%, 92%, 93%, 94%, 95%, 96%, 97%, 98%, 99%, or 100% sequence identity to an amino acid sequence selected from the group consisting of SEQ ID NOs: 28, 58, and 60-63;
an antibody comprising a complete H chain having at least 95% sequence identity to the amino acid sequence of SEQ ID NO: 74 or 79 and/or a complete L chain amino acid sequence having at least 90%, 91%, 92%, 93%, 94%, 95%, 96%, 97%, 98%, 99%, or 100% sequence identity to an amino acid sequence selected from the group consisting of SEQ ID NOs: 28, 58, 60-63, and 78;
an antibody comprising complete H/L chain sequences (respectively) comprising the amino acid sequences of SEQ ID NOs: 75/58 (h2A10.v2.T56A) or SEQ ID NOs: 79/78 (h2A10.v4);
an antibody that binds to the same epitope as an anti-TGFβ3 antibody comprising (a) HVR-H1 comprising the amino acid sequence of SEQ ID NO: 4; (b) HVR-H2 comprising the amino acid sequence of SEQ ID NO: 5; and (c) HVR-H3 comprising the amino acid sequence of SEQ ID NO:6;
an antibody that binds to the same epitope as an anti-TGFβ3 antibody comprising (a) HVR-H1 comprising the amino acid sequence of SEQ ID NO: 4; (b) HVR-H2 comprising the amino acid sequence of SEQ ID NO: 34; and (c) HVR-H3 comprising the amino acid sequence of SEQ ID NO:6;
an antibody that binds to the same epitope as an anti-TGFβ3 antibody comprising (a) HVR-H1 comprising the amino acid sequence of SEQ ID NO: 4; (b) HVR-H2 comprising the amino acid sequence of SEQ ID NO: 35; and (c) HVR-H3 comprising the amino acid sequence of SEQ ID NO:6;
an antibody that binds to the same epitope as an anti-TGFβ3 antibody comprising (a) HVR-H1 comprising the amino acid sequence of SEQ ID NO: 4; (b) HVR-H2 comprising the amino acid sequence of SEQ ID NO: 159; and (c) HVR-H3 comprising the amino acid sequence of SEQ ID NO:6;
an antibody that binds to the same epitope as an anti-TGFβ3 antibody comprising VH/VL sequences (respectively) selected from the group consisting of SEQ ID NOs: 53/36 (h2A10.v2.t56A), SEQ ID NOs: 57/56 (h2A10.v4), SEQ ID NOs: 51/36 (h2A10.v2.N54S), SEQ ID NOs: 55/54 (h2A10.v3), SEQ ID NOs: 52/36 (h2A10.v2.N54Q), SEQ ID NOs: 23/22 (rat 2A10), SEQ ID NOs: 37/36 (v1), SEQ ID NOs: 37/38 (v1.1), SEQ ID NOs: 37/39 (v1.2), SEQ ID NOs: 37/40 (v1.3), SEQ ID NOs: 37/41 (v1.4), SEQ ID NOs: 42/36 (v1.5), SEQ ID NOs: 43/36 (v1.6), SEQ ID NOs:44/36 (v1.7), SEQ ID NOs: 45/36 (v2), SEQ ID NOs: 45/38 (v2.1), SEQ ID NOs: 45/39 (v2.2), SEQ ID NOs: 45/40 (v2.3), SEQ ID NOs: 45/41 (v2.4), SEQ ID NOs: 46/36 (v2.5), SEQ ID NOs: 47/36 (v2.6), SEQ ID NOs: 48/36 (v2.7), SEQ ID NOs: 49/36 (v2.8), and SEQ ID NOs: 50/36 (v2.9);
an antibody that binds to the same epitope as an anti-TGFβ3 antibody comprising VH/VL sequences (respectively) selected from the group consisting of SEQ ID NOs: 53/36 (h2A10.v2.t56A), SEQ ID NOs: 57/56 (h2A10.v4), SEQ ID NOs: 51/36 (h2A10.v2.N54S), SEQ ID NOs: 55/54 (h2A10.v3), SEQ ID NOs: 52/36 (h2A10.v2.N54Q), SEQ ID NOs: 23/22 (rat 2A10), SEQ ID NOs: 37/36 (v1), SEQ ID NOs: 37/38 (v1.1), SEQ ID NOs: 37/39 (v1.2), SEQ ID NOs: 37/40 (v1.3), SEQ ID NOs: 37/41 (v1.4), SEQ ID NOs: 42/36 (v1.5), SEQ ID NOs: 43/36 (v1.6), SEQ ID NOs:44/36 (v1.7), SEQ ID NOs: 45/36 (v2), SEQ ID NOs: 45/38 (v2.1), SEQ ID NOs: 45/39 (v2.2), SEQ ID NOs: 45/40 (v2.3), SEQ ID NOs: 45/41 (v2.4), SEQ ID NOs: 46/36 (v2.5), SEQ ID NOs: 47/36 (v2.6), SEQ ID NOs: 48/36 (v2.7), SEQ ID NOs: 49/36 (v2.8), SEQ ID NOs: 50/36 (v2.9), SEQ ID NOs: 55/54 (v3), and SEQ ID NOs: 57/56 (v4);
an antibody that binds to the same epitope as an anti-TGFβ3 antibody comprising VH/VL sequences (respectively) of SEQ ID NOs: 57/56 (h2A10.v4);
an antibody that comprises an antigen binding domain that directly contacts amino acid residues R325, K331, W332, H334, E335, T387, I388, L389, Y391, V392, G393, R394, P396, K397, and V398 on human TGFβ3, wherein the anti-TGFβ3 antibody selectively neutralizes TGFβ3;
an antibody that selectively neutralizes TGFβ3 and has reduced toxicity in mice relative to the pan-TGFβ antibody 1D11;
an antibody that has reduced toxicity in mice relative to the pan-TGFβ antibody 1D11 at a dose of 50 mg/kg.

In some aspects of the above methods for treating a TGFβ-related disorder, the method comprises administering to a subject having systemic sclerosis (SSc) an effective amount of an anti-TGFβ3 antibody that selectively neutralizes TGFβ3 having one or more of the following properties:
(a) the anti-TGFβ3 antibody binds to the beta6/beta7 hairpin region of TGFβ3;
(b) binding of the anti-TGFβ3 antibody sterically blocks the ability of TGFBR2, but not TGFBR1, to bind TGFβ3;
(c) binding of the anti-TGFβ3 antibody to TGFβ3 blocks TGFBR2 binding and inhibits the TGFBR1/TGFBR2 signaling receptors from binding to TGFβ3;
(d) the anti-TGFβ3 antibody binds amino acid residue R394 in the beta6/beta7 hairpin region of human TGFβ3;
(e) the anti-TGFβ3 antibody binds amino acid residue R394 in the beta6/beta7 hairpin region of human TGFβ3 and residue R394 of TGFβ3 makes an ionic salt bridge with the anti-TGFβ3 antibody in the heavy chain CDR2;
(f) isoform selectivity of the anti-TGFβ3 antibody for TGFβ3 over TGFβ1 is achieved by direct contact by the antigen binding domain of the anti-TGFβ3 antibody with amino acid residues T387, L389, and T395 of TGFβ3 (human TGFβ3 numbering);
(g) the isoform selectivity of the anti-TGFβ3 antibody for TGFβ3 over TGFβ2 is achieved by direct contact by the antigen binding domain of the anti-TGFβ3 antibody with amino acid residues R325, R394, and V398 of TGFβ3 (human TGFβ3 numbering);
(h) the anti-TGFβ3 antibody has reduced toxicity relative to the pan-TGFβ antibody 1D11;
(i) the anti-TGFβ3 antibody has reduced toxicity in rodents or cynomolgus monkeys relative to the pan-TGFβ antibody 1D11;
(j) the anti-TGFβ3 antibody has reduced toxicity relative to the pan-TGFβ small molecule inhibitor galunisertib;
(k) the anti-TGFβ3 antibody has reduced toxicity in rodents relative to the pan-TGFβ small molecule inhibitor galunisertib;
(l) the anti-TGFβ3 antibody has reduced toxicity relative to the anti-TGFβ1 antibody CAT-192;
(m) the anti-TGFβ3 antibody has reduced toxicity relative to an isoform selective anti-TGFβ2 antibody and/or anti-TGFβ2/3 antibody;
(n) the anti-TGFβ3 antibody comprises: (i) heavy chain CDRs comprising CDR-H1, -H2 and -H3, wherein CDR-H1 has the amino acid sequence of SEQ ID NO: 4, CDR-H2 has the amino acid sequence of one of SEQ ID NOs: 5, 34, 35, and 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9;
(o) the anti-TGFβ3 antibody comprises an antigen binding domain that directly contacts amino acid residues R325, K331, W332, H334, E335, T387, I388, L389, Y391, V392, G393, R394, P396, K397, and V398 on human TGFβ3; and
(p) the anti-TGFβ3 antibody as in (o), wherein the antigen binding domain is within 15-8, 8, 8-5, 7-5, 6-5, or 5 angstroms of the TGFβ3 amino acid residues.

In a further aspect, the invention provides a method for treating SSc, the method comprising administering to a subject having SSc an effective amount of an isoform-selective anti-TGFβ antibody having one or more of the following properties:
an antibody that selectively neutralizes TGFβ3;
an antibody that binds to TGFβ3 with a K_{D} of less than 10 pM and/or has a cell-based IC₅₀ less than 250 pM;
an antibody that binds to TGFβ3 with a K_{D} of less than about 5 pM, about 4 pM, about 3 pM, or less than about 2 pM;
an antibody that has a cell-based IC₅₀ less of than about 200 pM, about 150 pM, about 100 pM, about 75 pM, or about 50 pM;
an antibody provided herein that has a cell-based IC₅₀ for inhibition (neutralization) of TGFβ3 of less than about 20 pM;
an antibody that selectively neutralizes TGFβ3 and has reduced toxicity in mice relative to the pan-TGFβ antibody 1D11;
an antibody that selectively neutralizes TGFβ3 and has an improved safety profile.

In a further aspect, the invention provides a method for treating SSc, the method comprising administering to a subject having SSc an effective amount of an anti-TGFβ3 antibody selected from the following:
an antibody comprising at least one, two, three, four, five, or six HVRs selected from (a) HVR-H1 comprising the amino acid sequence of SEQ ID NO: 4; (b) HVR-H2 comprising the amino acid sequence selected from SEQ ID NOs: 5, 34, 35, and 159; (c) HVR-H3 comprising the amino acid sequence of SEQ ID NO: 6; (d) HVR-L1 comprising the amino acid sequence of SEQ ID NO: 7; (e) HVR-L2 comprising the amino acid sequence of SEQ ID NO: 8; and (f) HVR-L3 comprising the amino acid sequence of SEQ ID NO: 9;
an antibody comprising (a) HVR-H1 comprising the amino acid sequence of SEQ ID NO: 4; (b) HVR-H2 comprising the amino acid sequence of SEQ ID NO: 5; (c) HVR-H3 comprising the amino acid sequence of SEQ ID NO: 6; (d) HVR-L1 comprising the amino acid sequence of SEQ ID NO: 7; (e) HVR-L2 comprising the amino acid sequence of SEQ ID NO: 8; and (f) HVR-L3 comprising an amino acid sequence selected from SEQ ID NO: 9;
an antibody comprising (a) HVR-H1 comprising the amino acid sequence of SEQ ID NO: 4; (b) HVR-H2 comprising the amino acid sequence of SEQ ID NO: 34; (c) HVR-H3 comprising the amino acid sequence of SEQ ID NO: 6; (d) HVR-L1 comprising the amino acid sequence of SEQ ID NO: 7; (e) HVR-L2 comprising the amino acid sequence of SEQ ID NO: 8; and (f) HVR-L3 comprising an amino acid sequence selected from SEQ ID NO: 9;
an antibody comprising (a) HVR-H1 comprising the amino acid sequence of SEQ ID NO: 4; (b) HVR-H2 comprising the amino acid sequence of SEQ ID NO: 35; (c) HVR-H3 comprising the amino acid sequence of SEQ ID NO: 6; (d) HVR-L1 comprising the amino acid sequence of SEQ ID NO: 7; (e) HVR-L2 comprising the amino acid sequence of SEQ ID NO: 8; and (f) HVR-L3 comprising an amino acid sequence selected from SEQ ID NO: 9; and
an antibody comprising (a) HVR-H1 comprising the amino acid sequence of SEQ ID NO: 4; (b) HVR-H2 comprising the amino acid sequence of SEQ ID NO: 159; (c) HVR-H3 comprising the amino acid sequence of SEQ ID NO: 6; (d) HVR-L1 comprising the amino acid sequence of SEQ ID NO: 7; (e) HVR-L2 comprising the amino acid sequence of SEQ ID NO: 8; and (f) HVR-L3 comprising an amino acid sequence selected from SEQ ID NO: 9.

In certain embodiments, any one or more amino acids of an anti-TGFβ3 antibody as provided above are substituted at the following HVR positions:
in HVR-H2 (SEQ ID NO: 5): at position N54 (e.g., N54S, N54Q) or T56 (e.g., T56A).

In certain embodiments, the substitutions are conservative substitutions, as provided herein.

In any of the above embodiments of methods for treating SSc, an anti-TGFβ3 antibody may be humanized. In one embodiment, an anti-TGFβ3 antibody comprises HVRs as in any of the above embodiments, and further comprises an acceptor human framework, e.g. a human immunoglobulin framework or a human consensus framework. In another embodiment, an anti-TGFβ3 antibody comprises HVRs as in any of the above embodiments, and further comprises a VH comprising FR modifications selected from the group consisting of 47L or 47W; 49A, 49S, or 49G; 73D or 73N; and 76N, 78D or 78L, 78A, or 78V. In some embodiments, the anti-TGFβ3 antibody comprises a VH, wherein the VH comprises the amino acid sequence of SEQ ID NO: 23, wherein the VH comprises a set of framework modifications selected from the group consisting of: (i) 47L, 49A in FR2, 78V in FR3 (h2A10.v1); (ii) 47L, 49A in FR2, 73D, 76S, 78V in FR3 (h2A10.v2); (iii) 47W in FR2 (h2A10.v1.5); (iv): 49G in FR2 (h2A10.v1.6); (v) 78A in FR3 (h2A10.v1.7); (vi) 47W in FR2 (h2A10.v2.5); (vii) 49S in FR2 (h2A10.v2.6); (viii) 73N in FR3 (h2A10.v2.7); (ix) 76N in FR3 (h2A10.v2.8); (x) 78L in FR3 (h2A10.v2.9); and (xi) 49S in FR2, 76N, 78L in FR3 (h2A10.v3 and h2A10.v4). In another embodiment, an anti-TGFβ3 antibody comprises HVRs as in any of the above embodiments, and further comprises a VL comprising FR modifications selected from the group consisting of: 4L or 4M, 38H or 38Q, 43A or 43Q, and 58V. In another embodiment, the antibody comprises the VL of SEQ ID NO: 22, wherein the VL comprises a set of framework modifications selected from the group consisting of: (i) 4L in FR1, 38H and 43Q in FR2, 58I in FR3 (h2A10.v1 and h2A10.v2); (ii) 4M in FR1 (h2A10.v1.1 and h2A10.v2.1); (iii) 38Q in FR2 (h2A10.v1.2 and h2A10.v2.2); (iv) 43A in FR2 (h2A10.v1.3 and h2A10.v2.3); (v) 58V in FR3 (h2A10.v1.4 and h2A10.v2.4); (vi) 38Q, 43A in FR2, 58V in FR3 (h2A10.v3 and h2A10.v4); (vii) 58V in FR3 (h2A10.v1.4 and h2A10.v2.4); and (vi) 38Q, 43A in FR2, 58V in FR3 (h2A10.v3 and h2A10.v4).

In a further aspect, the invention provides a method for treating SSc, the method comprising administering to a subject having SSc an effective amount of an anti-TGFβ3 antibody selected from the following:
an antibody comprising a heavy chain variable domain (VH) sequence having at least 90%, 91%, 92%, 93%, 94%, 95%, 96%, 97%, 98%, 99%, or 100% sequence identity to the amino acid sequence selected from the group consisting of SEQ ID NOs: 23, 37, and 42-50, and a light chain variable domain (VL) having at least 90%, 91%, 92%, 93%, 94%, 95%, 96%, 97%, 98%, 99%, or 100% sequence identity to the amino acid sequence selected from the group consisting of SEQ ID NOs: 22, 36, and 38-41;
an antibody comprising VH/VL sequences (respectively) selected from the group consisting of SEQ ID NOs: 23/22 (rat 2A10), SEQ ID NOs: 37/36 (v1), SEQ ID NOs: 37/38 (v1.1), SEQ ID NOs: 37/39 (v1.2), SEQ ID NOs: 37/40 (v1.3), SEQ ID NOs: 37/41 (v1.4), SEQ ID NOs: 42/36 (v1.5), SEQ ID NOs: 43/36 (v1.6), SEQ ID NOs:44/36 (v1.7), SEQ ID NOs: 45/36 (v2), SEQ ID NOs: 45/38 (v2.1), SEQ ID NOs: 45/39 (v2.2), SEQ ID NOs: 45/40 (v2.3), SEQ ID NOs: 45/41 (v2.4), SEQ ID NOs: 46/36 (v2.5), SEQ ID NOs: 47/36 (v2.6), SEQ ID NOs: 48/36 (v2.7), SEQ ID NOs: 49/36 (v2.8), and SEQ ID NOs: 50/36 (v2.9);
an antibody comprising a heavy chain variable domain (VH) sequence having at least 90%, 91%, 92%, 93%, 94%, 95%, 96%, 97%, 98%, 99%, or 100% sequence identity to the amino acid sequence selected from the group consisting of SEQ ID NOs: 23, 37, 42-50, 55, and 57, and a light chain variable domain (VL) having at least 90%, 91%, 92%, 93%, 94%, 95%, 96%, 97%, 98%, 99%, or 100% sequence identity to the amino acid sequence selected from the group consisting of SEQ ID NOs: 22, 36, 38-41, 54 and 56;
an antibody comprising VH/VL sequences (respectively) selected from the group consisting of SEQ ID NOs: 23/22 (rat 2A10), SEQ ID NOs: 37/36 (vi), SEQ ID NOs: 37/38 (v1.1), SEQ ID NOs: 37/39 (v1.2), SEQ ID NOs: 37/40 (v1.3), SEQ ID NOs: 37/41 (v1.4), SEQ ID NOs: 42/36 (v1.5), SEQ ID NOs: 43/36 (v1.6), SEQ ID NOs:44/36 (v1.7), SEQ ID NOs: 45/36 (v2), SEQ ID NOs: 45/38 (v2.1), SEQ ID NOs: 45/39 (v2.2), SEQ ID NOs: 45/40 (v2.3), SEQ ID NOs: 45/41 (v2.4), SEQ ID NOs: 46/36 (v2.5), SEQ ID NOs: 47/36 (v2.6), SEQ ID NOs: 48/36 (v2.7), SEQ ID NOs: 49/36 (v2.8), SEQ ID NOs: 50/36 (v2.9), SEQ ID NOs: 55/54 (v3), and SEQ ID NOs: 57/56 (v4);
an antibody comprises a complete H chain amino acid sequence having at least 95% sequence identity to the amino acid sequence selected from the group consisting of SEQ ID NOs: 29, 59, 64, and 65-72 and/or a complete L chain amino acid sequence having an amino acid sequence selected from the group consisting of SEQ ID NOs: 28, 58, and 60-63;
an antibody comprising a complete H/L chain pair, the complete H/L chain pair (respectively) comprising amino acid sequences selected from the group consisting of SEQ ID NOs: 29/28 (rat 2A10), SEQ ID NOs: 59/58 (v1), SEQ ID NOs: 59/60 (v1.1), SEQ ID NOs: 59/61 (v1.2), SEQ ID NOs: 59/62 (v1.3), SEQ ID NOs: 59/63 (v1.4), SEQ ID NOs: 64/58 (v1.5), SEQ ID NOs: 65/58 (v1.6), SEQ ID NOs: 66/58 (v1.7), SEQ ID NOs: 67/58 (v2), SEQ ID NOs: 67/60 (v2.1), SEQ ID NOs: 67/61 (v2.2), SEQ ID NOs: 67/62 (v2.3), SEQ ID NOs: 67/63 (v2.4), SEQ ID NOs: 68/58 (v2.5), SEQ ID NOs: 69/58 (v2.6), SEQ ID NOs: 70/58 (v2.7), SEQ ID NOs: 71/58 (v2.8), SEQ ID NOs: 72/58 (v2.9);
an antibody comprises a complete H chain amino acid sequence having at least 95% sequence identity to the amino acid sequence selected from the group consisting of SEQ ID NOs: 29, 59, 64, 65-72, 77 and 79, and/or a complete L chain amino acid sequence having an amino acid sequence selected from the group consisting of SEQ ID NOs: 28, 58, 60-63, 76 and 78;
an antibody comprising a complete H/L chain pair, the complete H/L chain pair (respectively) comprising amino acid sequences selected from the group consisting of SEQ ID NOs: 29/28 (rat 2A10), SEQ ID NOs: 59/58 (v1), SEQ ID NOs: 59/60 (v1.1), SEQ ID NOs: 59/61 (v1.2), SEQ ID NOs: 59/62 (v1.3), SEQ ID NOs: 59/63 (v1.4), SEQ ID NOs: 64/58 (v1.5), SEQ ID NOs: 65/58 (v1.6), SEQ ID NOs: 66/58 (v1.7), SEQ ID NOs: 67/58 (v2), SEQ ID NOs: 67/60 (v2.1), SEQ ID NOs: 67/61 (v2.2), SEQ ID NOs: 67/62 (v2.3), SEQ ID NOs: 67/63 (v2.4), SEQ ID NOs: 68/58 (v2.5), SEQ ID NOs: 69/58 (v2.6), SEQ ID NOs: 70/58 (v2.7), SEQ ID NOs: 71/58 (v2.8), SEQ ID NOs: 72/58 (v2.9), SEQ ID NOs: 77/76 (v3), and SEQ ID NOs: 79/78 (v4);
an antibody comprising a VH having at least 90%, 91%, 92%, 93%, 94%, 95%, 96%, 97%, 98%, 99%, or 100% sequence identity to the amino acid sequence of SEQ ID NO: 52 and a VL amino acid sequence having at least 90%, 91%, 92%, 93%, 94%, 95%, 96%, 97%, 98%, 99%, or 100% sequence identity to an amino acid sequence selected from the group consisting of SEQ ID NOs: 22, 36, and 38-41;
an antibody comprising VH/VL sequences (respectively) comprising the amino acid sequences of SEQ ID NOs: 52/36 (h2A10.v2.N54Q);
an antibody comprising a complete H chain having at least 90%, 91%, 92%, 93%, 94%, 95%, 96%, 97%, 98%, 99%, or 100% sequence identity to the amino acid sequence of SEQ ID NO: 74 and a complete L chain amino acid sequence having at least 90%, 91%, 92%, 93%, 94%, 95%, 96%, 97%, 98%, 99%, or 100% sequence identity to an amino acid sequence selected from the group consisting of SEQ ID NOs: 28, 58, and 60-63;
an antibody comprising complete H/L chain sequences (respectively) comprising the amino acid sequences of SEQ ID NOs: 74/58 (h2A10.v2.N54Q);
an antibody comprising a VH having at least 90%, 91%, 92%, 93%, 94%, 95%, 96%, 97% 98%, 99%, or 100% sequence identity to the amino acid sequence of SEQ ID NO: 51 or 55 and a VL amino acid sequence having at least 90%, 91%, 92%, 93%, 94%, 95%, 96%, 97%, 98%, 99%, or 100% sequence identity to an amino acid sequence selected from the group consisting of SEQ ID NOs: 22, 36, and 38-41;
an antibody comprising a VH having at least 90%, 91%, 92%, 93%, 94%, 95%, 96%, 97%, 98%, 99%, or 100% sequence identity to the amino acid sequence of SEQ ID NO: 51 or 55 and a VL amino acid sequence having at least 90%, 91%, 92%, 93%, 94%, 95%, 96%, 97%, 98%, 99%, or 100% sequence identity to an amino acid sequence selected from the group consisting of SEQ ID NOs: 22, 36, 38-41, and 54;
an antibody comprising VH/VL sequences (respectively) comprising the amino acid sequences of SEQ ID NO: 51/36 (h2A10.v2.N54S) or SEQ ID NO: 55/54 (h2A10.v3);
an antibody comprising a complete H chain having at least 95% sequence identity to the amino acid sequence of SEQ ID NO: 73 or 77 and/or the anti-TGFβ3 antibody comprises a complete L chain amino acid sequence having at least 90%, 91%, 92%, 93%, 94%, 95%, 96%, 97%, 98%, 99%, or 100% sequence identity to an amino acid sequence selected from the group consisting of SEQ ID NOs: 28, 58, and 60-63;
an antibody comprising a complete H chain having at least 95% sequence identity to the amino acid sequence of SEQ ID NO: 73 or 77 and/or the anti-TGFβ3 antibody comprises a complete L chain amino acid sequence having at least 90%, 91%, 92%, 93%, 94%, 95%, 96%, 97%, 98%, 99%, or 100% sequence identity to an amino acid sequence selected from the group consisting of SEQ ID NOs: 28, 58, 60-63, and 76;
an antibody comprising complete H/L chain sequences (respectively) comprising the amino acid sequences of SEQ ID NOs: 73/58 (h2A10.v2.N54S) or SEQ ID NOs: 77/76 (h2A10.v3);
an antibody comprising a VH having at least 90%, 91%, 92%, 93%, 94%, 95%, 96%, 97%, 98%, 99%, or 100% sequence identity to the amino acid sequence of SEQ ID NO: 53 or 57, and a VL amino acid sequence having at least 90%, 91%, 92%, 93%, 94%, 95%, 96%, 97%, 98%, 99%, or 100% sequence identity to an amino acid sequence selected from the group consisting of SEQ ID NOs: 22, 36, and 38-41;
an antibody comprising a VH having at least 90%, 91%, 92%, 93%, 94%, 95%, 96%, 97%, 98%, 99%, or 100% sequence identity to the amino acid sequence of SEQ ID NO: 53 or 57, and a VL amino acid sequence having at least 90%, 91%, 92%, 93%, 94%, 95%, 96%, 97%, 98%, 99%, or 100% sequence identity to an amino acid sequence selected from the group consisting of SEQ ID NOs: 22, 36, 38-41, and 56;
an antibody comprising VH/VL sequences (respectively) comprising the amino acid sequences of SEQ ID NOs: 53/36 (h2A10.v2.T56A) or SEQ ID NOs: 57/56 (h2A10.v4);
an antibody comprising a complete H chain having at least 95% sequence identity to the amino acid sequence of SEQ ID NO: 74 or 79 and/or a complete L chain amino acid sequence having at least 90%, 91%, 92%, 93%, 94%, 95%, 96%, 97%, 98%, 99%, or 100% sequence identity to an amino acid sequence selected from the group consisting of SEQ ID NOs: 28, 58, and 60-63;
an antibody comprising a complete H chain having at least 95% sequence identity to the amino acid sequence of SEQ ID NO: 74 or 79 and/or a complete L chain amino acid sequence having at least 90%, 91%, 92%, 93%, 94%, 95%, 96%, 97%, 98%, 99%, or 100% sequence identity to an amino acid sequence selected from the group consisting of SEQ ID NOs: 28, 58, 60-63, and 78;
an antibody comprising complete H/L chain sequences (respectively) comprising the amino acid sequences of SEQ ID NOs: 75/58 (h2A10.v2.T56A) or SEQ ID NOs: 79/78 (h2A10.v4);
an antibody that binds to the same epitope as an anti-TGFβ3 antibody comprising (a) HVR-H1 comprising the amino acid sequence of SEQ ID NO: 4; (b) HVR-H2 comprising the amino acid sequence of SEQ ID NO: 5; and (c) HVR-H3 comprising the amino acid sequence of SEQ ID NO:6;
an antibody that binds to the same epitope as an anti-TGFβ3 antibody comprising (a) HVR-H1 comprising the amino acid sequence of SEQ ID NO: 4; (b) HVR-H2 comprising the amino acid sequence of SEQ ID NO: 34; and (c) HVR-H3 comprising the amino acid sequence of SEQ ID NO:6;
an antibody that binds to the same epitope as an anti-TGFβ3 antibody comprising (a) HVR-H1 comprising the amino acid sequence of SEQ ID NO: 4; (b) HVR-H2 comprising the amino acid sequence of SEQ ID NO: 35; and (c) HVR-H3 comprising the amino acid sequence of SEQ ID NO:6;
an antibody that binds to the same epitope as an anti-TGFβ3 antibody comprising (a) HVR-H1 comprising the amino acid sequence of SEQ ID NO: 4; (b) HVR-H2 comprising the amino acid sequence of SEQ ID NO: 159; and (c) HVR-H3 comprising the amino acid sequence of SEQ ID NO:6;
an antibody that binds to the same epitope as an anti-TGFβ3 antibody comprising VH/VL sequences (respectively) selected from the group consisting of SEQ ID NOs: 53/36 (h2A10.v2.t56A), SEQ ID NOs: 57/56 (h2A10.v4), SEQ ID NOs: 51/36 (h2A10.v2.N54S), SEQ ID NOs: 55/54 (h2A10.v3), SEQ ID NOs: 52/36 (h2A10.v2.N54Q), SEQ ID NOs: 23/22 (rat 2A10), SEQ ID NOs: 37/36 (v1), SEQ ID NOs: 37/38 (v1.1), SEQ ID NOs: 37/39 (v1.2), SEQ ID NOs: 37/40 (v1.3), SEQ ID NOs: 37/41 (v1.4), SEQ ID NOs: 42/36 (v1.5), SEQ ID NOs: 43/36 (v1.6), SEQ ID NOs:44/36 (v1.7), SEQ ID NOs: 45/36 (v2), SEQ ID NOs: 45/38 (v2.1), SEQ ID NOs: 45/39 (v2.2), SEQ ID NOs: 45/40 (v2.3), SEQ ID NOs: 45/41 (v2.4), SEQ ID NOs: 46/36 (v2.5), SEQ ID NOs: 47/36 (v2.6), SEQ ID NOs: 48/36 (v2.7), SEQ ID NOs: 49/36 (v2.8), and SEQ ID NOs: 50/36 (v2.9);
an antibody that binds to the same epitope as an anti-TGFβ3 antibody comprising VH/VL sequences (respectively) selected from the group consisting of SEQ ID NOs: 53/36 (h2A10.v2.t56A), SEQ ID NOs: 57/56 (h2A10.v4), SEQ ID NOs: 51/36 (h2A10.v2.N54S), SEQ ID NOs: 55/54 (h2A10.v3), SEQ ID NOs: 52/36 (h2A10.v2.N54Q), SEQ ID NOs: 23/22 (rat 2A10), SEQ ID NOs: 37/36 (vi), SEQ ID NOs: 37/38 (v1.1), SEQ ID NOs: 37/39 (v1.2), SEQ ID NOs: 37/40 (v1.3), SEQ ID NOs: 37/41 (v1.4), SEQ ID NOs: 42/36 (v1.5), SEQ ID NOs: 43/36 (v1.6), SEQ ID NOs:44/36 (v1.7), SEQ ID NOs: 45/36 (v2), SEQ ID NOs: 45/38 (v2.1), SEQ ID NOs: 45/39 (v2.2), SEQ ID NOs: 45/40 (v2.3), SEQ ID NOs: 45/41 (v2.4), SEQ ID NOs: 46/36 (v2.5), SEQ ID NOs: 47/36 (v2.6), SEQ ID NOs: 48/36 (v2.7), SEQ ID NOs: 49/36 (v2.8), SEQ ID NOs: 50/36 (v2.9), SEQ ID NOs: 55/54 (v3) and SEQ ID NOs: 57/56 (v4);
an antibody that binds to the same epitope as an anti-TGFβ3 antibody comprising VH/VL sequences (respectively) of SEQ ID NOs: 57/56 (h2A10.v4);
an antibody that comprises an antigen binding domain that directly contacts amino acid residues R325, K331, W332, H334, E335, T387, I388, L389, Y391, V392, G393, R394, P396, K397, and V398 on human TGFβ3, wherein the anti-TGFβ3 antibody selectively neutralizes TGFβ3;
an antibody that selectively neutralizes TGFβ3 and has reduced toxicity in mice relative to the pan-TGFβ antibody 1D11;
an antibody that has reduced toxicity in mice relative to the pan-TGFβ antibody 1D11 at a dose of 50 mg/kg.

In some aspects of the above methods for treating a TGFβ-related disorder, the method comprises administering to a subject having idiopathic pulmonary fibrosis (IPF) an effective amount of an anti-TGFβ3 antibody that selectively neutralizes TGFβ3 having one or more of the following properties:
(a) the anti-TGFβ3 antibody binds to the beta6/beta7 hairpin region of TGFβ3;
(b) binding of the anti-TGFβ3 antibody sterically blocks the ability of TGFBR2, but not TGFBR1, to bind TGFβ3;
(c) binding of the anti-TGFβ3 antibody to TGFβ3 blocks TGFBR2 binding and inhibits the TGFBR1/TGFBR2 signaling receptors from binding to TGFβ3;
(d) the anti-TGFβ3 antibody binds amino acid residue R394 in the beta6/beta7 hairpin region of human TGFβ3;
(e) the anti-TGFβ3 antibody binds amino acid residue R394 in the beta6/beta7 hairpin region of human TGFβ3 and residue R394 of TGFβ3 makes an ionic salt bridge with the anti-TGFβ3 antibody in the heavy chain CDR2;
(f) isoform selectivity of the anti-TGFβ3 antibody for TGFβ3 over TGFβ1 is achieved by direct contact by the antigen binding domain of the anti-TGFβ3 antibody with amino acid residues T387, L389, and T395 of TGFβ3 (human TGFβ3 numbering);
(g) the isoform selectivity of the anti-TGFβ3 antibody for TGFβ3 over TGFβ2 is achieved by direct contact by the antigen binding domain of the anti-TGFβ3 antibody with amino acid residues R325, R394, and V398 of TGFβ3 (human TGFβ3 numbering);
(h) the anti-TGFβ3 antibody has reduced toxicity relative to the pan-TGFβ antibody 1D11;
(i) the anti-TGFβ3 antibody has reduced toxicity in rodents or cynomolgus monkeys relative to the pan-TGFβ antibody 1D11;
(j) the anti-TGFβ3 antibody has reduced toxicity relative to the pan-TGFβ small molecule inhibitor galunisertib;
(k) the anti-TGFβ3 antibody has reduced toxicity in rodents relative to the pan-TGFβ small molecule inhibitor galunisertib;
(l) the anti-TGFβ3 antibody has reduced toxicity relative to the anti-TGFβ1 antibody CAT-192;
(m) the anti-TGFβ3 antibody has reduced toxicity relative to an isoform selective anti-TGFβ2 antibody and/or anti-TGFβ2/3 antibody;
(n) the anti-TGFβ3 antibody comprises: (i) heavy chain CDRs comprising CDR-H1, -H2 and -H3, wherein CDR-H1 has the amino acid sequence of SEQ ID NO: 4, CDR-H2 has the amino acid sequence of one of SEQ ID NOs: 5, 34, 35, and 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9;
(o) the anti-TGFβ3 antibody comprises an antigen binding domain that directly contacts amino acid residues R325, K331, W332, H334, E335, T387, I388, L389, Y391, V392, G393, R394, P396, K397, and V398 on human TGFβ3; and
(p) the anti-TGFβ3 antibody as in (o), wherein the antigen binding domain is within 15-8, 8, 8-5, 7-5, 6-5, or 5 angstroms of the TGFβ3 amino acid residues.

In a further aspect, the invention provides a method for treating IPF, the method comprising administering to a subject having IPF an effective amount of an isoform-selective anti-TGFβ antibody having one or more of the following properties:
an antibody that selectively neutralizes TGFβ3;
an antibody that binds to TGFβ3 with a K_{D} of less than 10 pM and/or has a cell-based IC₅₀ less than 250 pM;
an antibody that binds to TGFβ3 with a K_{D} of less than about 5 pM, about 4 pM, about 3 pM, or less than about 2 pM;
an antibody that has a cell-based IC₅₀ less of than about 200 pM, about 150 pM, about 100 pM, about 75 pM, or about 50 pM;
an antibody provided herein that has a cell-based IC₅₀ for inhibition (neutralization) of TGFβ3 of less than about 20 pM;
an antibody that selectively neutralizes TGFβ3 and has reduced toxicity in mice relative to the pan-TGFβ antibody 1D11;
an antibody that selectively neutralizes TGFβ3 and has an improved safety profile.

In a further aspect, the invention provides a method for treating IPF, the method comprising administering to a subject having IPF an effective amount of an anti-TGFβ3 antibody selected from the following:
an antibody comprising at least one, two, three, four, five, or six HVRs selected from (a) HVR-H1 comprising the amino acid sequence of SEQ ID NO: 4; (b) HVR-H2 comprising the amino acid sequence selected from SEQ ID NOs: 5, 34, 35, and 159; (c) HVR-H3 comprising the amino acid sequence of SEQ ID NO: 6; (d) HVR-L1 comprising the amino acid sequence of SEQ ID NO: 7; (e) HVR-L2 comprising the amino acid sequence of SEQ ID NO: 8; and (f) HVR-L3 comprising the amino acid sequence of SEQ ID NO: 9;
an antibody comprising (a) HVR-H1 comprising the amino acid sequence of SEQ ID NO: 4; (b) HVR-H2 comprising the amino acid sequence of SEQ ID NO: 5; (c) HVR-H3 comprising the amino acid sequence of SEQ ID NO: 6; (d) HVR-L1 comprising the amino acid sequence of SEQ ID NO: 7; (e) HVR-L2 comprising the amino acid sequence of SEQ ID NO: 8; and (f) HVR-L3 comprising an amino acid sequence selected from SEQ ID NO: 9;
an antibody comprising (a) HVR-H1 comprising the amino acid sequence of SEQ ID NO: 4; (b) HVR-H2 comprising the amino acid sequence of SEQ ID NO: 34; (c) HVR-H3 comprising the amino acid sequence of SEQ ID NO: 6; (d) HVR-L1 comprising the amino acid sequence of SEQ ID NO: 7; (e) HVR-L2 comprising the amino acid sequence of SEQ ID NO: 8; and (f) HVR-L3 comprising an amino acid sequence selected from SEQ ID NO: 9;
an antibody comprising (a) HVR-H1 comprising the amino acid sequence of SEQ ID NO: 4; (b) HVR-H2 comprising the amino acid sequence of SEQ ID NO: 35; (c) HVR-H3 comprising the amino acid sequence of SEQ ID NO: 6; (d) HVR-L1 comprising the amino acid sequence of SEQ ID NO: 7; (e) HVR-L2 comprising the amino acid sequence of SEQ ID NO: 8; and (f) HVR-L3 comprising an amino acid sequence selected from SEQ ID NO: 9; and
an antibody comprising (a) HVR-H1 comprising the amino acid sequence of SEQ ID NO: 4; (b) HVR-H2 comprising the amino acid sequence of SEQ ID NO: 159; (c) HVR-H3 comprising the amino acid sequence of SEQ ID NO: 6; (d) HVR-L1 comprising the amino acid sequence of SEQ ID NO: 7; (e) HVR-L2 comprising the amino acid sequence of SEQ ID NO: 8; and (f) HVR-L3 comprising an amino acid sequence selected from SEQ ID NO: 9.

In certain embodiments, any one or more amino acids of an anti-TGFβ3 antibody as provided above are substituted at the following HVR positions:
in HVR-H2 (SEQ ID NO: 5): at position N54 (e.g., N54S, N54Q) or T56 (e.g., T56A).

In certain embodiments, the substitutions are conservative substitutions, as provided herein.

In any of the above embodiments of methods for treating IPF, an anti-TGFβ3 antibody may be humanized. In one embodiment, an anti-TGFβ3 antibody comprises HVRs as in any of the above embodiments, and further comprises an acceptor human framework, e.g. a human immunoglobulin framework or a human consensus framework. In another embodiment, an anti-TGFβ3 antibody comprises HVRs as in any of the above embodiments, and further comprises a VH comprising FR modifications selected from the group consisting of 47L or 47W; 49A, 49S, or 49G; 73D or 73N; and 76N, 78D or 78L, 78A, or 78V. In some embodiments, the anti-TGFβ3 antibody comprises a VH, wherein the VH comprises the amino acid sequence of SEQ ID NO: 23, wherein the VH comprises a set of framework modifications selected from the group consisting of: (i) 47L, 49A in FR2, 78V in FR3 (h2A10.v1); (ii) 47L, 49A in FR2, 73D, 76S, 78V in FR3 (h2A10.v2); (iii) 47W in FR2 (h2A10.v1.5); (iv): 49G in FR2 (h2A10.v1.6); (v) 78A in FR3 (h2A10.v1.7); (vi) 47W in FR2 (h2A10.v2.5); (vii) 49S in FR2 (h2A10.v2.6); (viii) 73N in FR3 (h2A10.v2.7); (ix) 76N in FR3 (h2A10.v2.8); (x) 78L in FR3 (h2A10.v2.9); and (xi) 49S in FR2, 76N, 78L in FR3 (h2A10.v3 and h2A10.v4). In another embodiment, an anti-TGFβ3 antibody comprises HVRs as in any of the above embodiments, and further comprises a VL comprising FR modifications selected from the group consisting of: 4L or 4M, 38H or 38Q, 43A or 43Q, and 58V. In another embodiment, the antibody comprises the VL of SEQ ID NO: 22, wherein the VL comprises a set of framework modifications selected from the group consisting of: (i) 4L in FR1, 38H and 43Q in FR2, 58I in FR3 (h2A10.v1 and h2A10.v2); (ii) 4M in FR1 (h2A10.v1.1 and h2A10.v2.1); (iii) 38Q in FR2 (h2A10.v1.2 and h2A10.v2.2); (iv) 43A in FR2 (h2A10.v1.3 and h2A10.v2.3); (v) 58V in FR3 (h2A10.v1.4 and h2A10.v2.4); (vi) 38Q, 43A in FR2, 58V in FR3 (h2A10.v3 and h2A10.v4); (vii) 58V in FR3 (h2A10.v1.4 and h2A10.v2.4); and (vi) 38Q, 43A in FR2, 58V in FR3 (h2A10.v3 and h2A10.v4).

In a further aspect, the invention provides a method for treating IPF, the method comprising administering to a subject having IPF an effective amount of an anti-TGFβ3 antibody selected from the following:
an antibody comprising a heavy chain variable domain (VH) sequence having at least 90%, 91%, 92%, 93%, 94%, 95%, 96%, 97%, 98%, 99%, or 100% sequence identity to the amino acid sequence selected from the group consisting of SEQ ID NOs: 23, 37, and 42-50, and a light chain variable domain (VL) having at least 90%, 91%, 92%, 93%, 94%, 95%, 96%, 97%, 98%, 99%, or 100% sequence identity to the amino acid sequence selected from the group consisting of SEQ ID NOs: 22, 36, and 38-41;
an antibody comprising VH/VL sequences (respectively) selected from the group consisting of SEQ ID NOs: 23/22 (rat 2A10), SEQ ID NOs: 37/36 (v1), SEQ ID NOs: 37/38 (v1.1), SEQ ID NOs: 37/39 (v1.2), SEQ ID NOs: 37/40 (v1.3), SEQ ID NOs: 37/41 (v1.4), SEQ ID NOs: 42/36 (v1.5), SEQ ID NOs: 43/36 (v1.6), SEQ ID NOs:44/36 (v1.7), SEQ ID NOs: 45/36 (v2), SEQ ID NOs: 45/38 (v2.1), SEQ ID NOs: 45/39 (v2.2), SEQ ID NOs: 45/40 (v2.3), SEQ ID NOs: 45/41 (v2.4), SEQ ID NOs: 46/36 (v2.5), SEQ ID NOs: 47/36 (v2.6), SEQ ID NOs: 48/36 (v2.7), SEQ ID NOs: 49/36 (v2.8), and SEQ ID NOs: 50/36 (v2.9);
an antibody comprising a heavy chain variable domain (VH) sequence having at least 90%, 91%, 92%, 93%, 94%, 95%, 96%, 97%, 98%, 99%, or 100% sequence identity to the amino acid sequence selected from the group consisting of SEQ ID NOs: 23, 37, 42-50, 55, and 57, and a light chain variable domain (VL) having at least 90%, 91%, 92%, 93%, 94%, 95%, 96%, 97%, 98%, 99%, or 100% sequence identity to the amino acid sequence selected from the group consisting of SEQ ID NOs: 22, 36, 38-41, 54, and 56;
an antibody comprising VH/VL sequences (respectively) selected from the group consisting of SEQ ID NOs: 23/22 (rat 2A10), SEQ ID NOs: 37/36 (v1), SEQ ID NOs: 37/38 (v1.1), SEQ ID NOs: 37/39 (v1.2), SEQ ID NOs: 37/40 (v1.3), SEQ ID NOs: 37/41 (v1.4), SEQ ID NOs: 42/36 (v1.5), SEQ ID NOs: 43/36 (v1.6), SEQ ID NOs:44/36 (v1.7), SEQ ID NOs: 45/36 (v2), SEQ ID NOs: 45/38 (v2.1), SEQ ID NOs: 45/39 (v2.2), SEQ ID NOs: 45/40 (v2.3), SEQ ID NOs: 45/41 (v2.4), SEQ ID NOs: 46/36 (v2.5), SEQ ID NOs: 47/36 (v2.6), SEQ ID NOs: 48/36 (v2.7), SEQ ID NOs: 49/36 (v2.8), SEQ ID NOs: 50/36 (v2.9), SEQ ID NOs: 55/54 (v3), and SEQ ID NOs: 57/56 (v4);
an antibody comprises a complete H chain amino acid sequence having at least 95% sequence identity to the amino acid sequence selected from the group consisting of SEQ ID NOs: 29, 59, 64, and 65-72 and/or a complete L chain amino acid sequence having an amino acid sequence selected from the group consisting of SEQ ID NOs: 28, 58, and 60-63;
an antibody comprising a complete H/L chain pair, the complete H/L chain pair (respectively) comprising amino acid sequences selected from the group consisting of SEQ ID NOs: 29/28 (rat 2A10), SEQ ID NOs: 59/58 (v1), SEQ ID NOs: 59/60 (v1.1), SEQ ID NOs: 59/61 (v1.2), SEQ ID NOs: 59/62 (v1.3), SEQ ID NOs: 59/63 (v1.4), SEQ ID NOs: 64/58 (v1.5), SEQ ID NOs: 65/58 (v1.6), SEQ ID NOs: 66/58 (v1.7), SEQ ID NOs: 67/58 (v2), SEQ ID NOs: 67/60 (v2.1), SEQ ID NOs: 67/61 (v2.2), SEQ ID NOs: 67/62 (v2.3), SEQ ID NOs: 67/63 (v2.4), SEQ ID NOs: 68/58 (v2.5), SEQ ID NOs: 69/58 (v2.6), SEQ ID NOs: 70/58 (v2.7), SEQ ID NOs: 71/58 (v2.8), SEQ ID NOs: 72/58 (v2.9);
an antibody comprises a complete H chain amino acid sequence having at least 95% sequence identity to the amino acid sequence selected from the group consisting of SEQ ID NOs: 29, 59, 64, 65-72, 77 and 79, and/or a complete L chain amino acid sequence having an amino acid sequence selected from the group consisting of SEQ ID NOs: 28, 58, 60-63, 76, and 78;
an antibody comprising a complete H/L chain pair, the complete H/L chain pair (respectively) comprising amino acid sequences selected from the group consisting of SEQ ID NOs: 29/28 (rat 2A10), SEQ ID NOs: 59/58 (v1), SEQ ID NOs: 59/60 (v1.1), SEQ ID NOs: 59/61 (v1.2), SEQ ID NOs: 59/62 (v1.3), SEQ ID NOs: 59/63 (v1.4), SEQ ID NOs: 64/58 (v1.5), SEQ ID NOs: 65/58 (v1.6), SEQ ID NOs: 66/58 (v1.7), SEQ ID NOs: 67/58 (v2), SEQ ID NOs: 67/60 (v2.1), SEQ ID NOs: 67/61 (v2.2), SEQ ID NOs: 67/62 (v2.3), SEQ ID NOs: 67/63 (v2.4), SEQ ID NOs: 68/58 (v2.5), SEQ ID NOs: 69/58 (v2.6), SEQ ID NOs: 70/58 (v2.7), SEQ ID NOs: 71/58 (v2.8), SEQ ID NOs: 72/58 (v2.9), SEQ ID NOs: 77/76 (v3), and SEQ ID NOs: 79/78;
an antibody comprising a VH having at least 90%, 91%, 92%, 93%, 94%, 95%, 96%, 97%, 98%, 99%, or 100% sequence identity to the amino acid sequence of SEQ ID NO: 52 and a VL amino acid sequence having at least 90%, 91%, 92%, 93%, 94%, 95%, 96%, 97%, 98%, 99%, or 100% sequence identity to an amino acid sequence selected from the group consisting of SEQ ID NOs: 22, 36, and 38-41;
an antibody comprising VH/VL sequences (respectively) comprising the amino acid sequences of SEQ ID NOs: 52/36 (h2A10.v2.N54Q);
an antibody comprising a complete H chain having at least 90%, 91%, 92%, 93%, 94%, 95%, 96%, 97%, 98%, 99%, or 100% sequence identity to the amino acid sequence of SEQ ID NO: 74 and a complete L chain amino acid sequence having at least 90%, 91%, 92%, 93%, 94%, 95%, 96%, 97%, 98%, 99%, or 100% sequence identity to an amino acid sequence selected from the group consisting of SEQ ID NOs: 28, 58, and 60-63;
an antibody comprising complete H/L chain sequences (respectively) comprising the amino acid sequences of SEQ ID NOs: 74/58 (h2A10.v2.N54Q);
an antibody comprising a VH having at least 90%, 91%, 92%, 93%, 94%, 95%, 96%, 97%, 98%, 99%, or 100% sequence identity to the amino acid sequence of SEQ ID NO: 51 or 55 and a VL amino acid sequence having at least 90%, 91%, 92%, 93%, 94%, 95%, 96%, 97%, 98%, 99%, or 100% sequence identity to an amino acid sequence selected from the group consisting of SEQ ID NOs: 22, 36, and 38-41;
an antibody comprising a VH having at least 90%, 91%, 92%, 93%, 94%, 95%, 96%, 97% 98%, 99%, or 100% sequence identity to the amino acid sequence of SEQ ID NO: 51 or 55 and a VL amino acid sequence having at least 90%, 91%, 92%, 93%, 94%, 95%, 96%, 97%, 98%, 99%, or 100% sequence identity to an amino acid sequence selected from the group consisting of SEQ ID NOs: 22, 36, 38-41, and 54;
an antibody comprising VH/VL sequences (respectively) comprising the amino acid sequences of SEQ ID NO: 51/36 (h2A10.v2.N54S) or SEQ ID NO: 55/54 (h2A10.v3);
an antibody comprising a complete H chain having at least 95% sequence identity to the amino acid sequence of SEQ ID NO: 73 or 77 and/or the anti-TGFβ3 antibody comprises a complete L chain amino acid sequence having at least 90%, 91%, 92%, 93%, 94%, 95%, 96%, 97%, 98%, 99%, or 100% sequence identity to an amino acid sequence selected from the group consisting of SEQ ID NOs: 28, 58, and 60-63;
an antibody comprising a complete H chain having at least 95% sequence identity to the amino acid sequence of SEQ ID NO: 73 or 77 and/or the anti-TGFβ3 antibody comprises a complete L chain amino acid sequence having at least 90%, 91%, 92%, 93%, 94%, 95%, 96%, 97%, 98%, 99%, or 100% sequence identity to an amino acid sequence selected from the group consisting of SEQ ID NOs: 28, 58, 60-63, and 76;
an antibody comprising complete H/L chain sequences (respectively) comprising the amino acid sequences of SEQ ID NOs: 73/58 (h2A10.v2.N54S) or SEQ ID NOs: 77/76 (h2A10.v3);
an antibody comprising a VH having at least 90%, 91%, 92%, 93%, 94%, 95%, 96%, 97% 98%, 99%, or 100% sequence identity to the amino acid sequence of SEQ ID NO: 53 or 57, and a VL amino acid sequence having at least 90%, 91%, 92%, 93%, 94%, 95%, 96%, 97%, 98%, 99%, or 100% sequence identity to an amino acid sequence selected from the group consisting of SEQ ID NOs: 22, 36, and 38-41;
an antibody comprising a VH having at least 90%, 91%, 92%, 93%, 94%, 95%, 96%, 97%, 98%, 99%, or 100% sequence identity to the amino acid sequence of SEQ ID NO: 53 or 57, and a VL amino acid sequence having at least 90%, 91%, 92%, 93%, 94%, 95%, 96%, 97%, 98%, 99%, or 100% sequence identity to an amino acid sequence selected from the group consisting of SEQ ID NOs: 22, 36, 38-41, and 56;
an antibody comprising VH/VL sequences (respectively) comprising the amino acid sequences of SEQ ID NOs: 53/36 (h2A10.v2.T56A) or SEQ ID NOs: 57/56 (h2A10.v4);
an antibody comprising a complete H chain having at least 95% sequence identity to the amino acid sequence of SEQ ID NO: 74 or 79 and/or a complete L chain amino acid sequence having at least 90%, 91%, 92%, 93%, 94%, 95%, 96%, 97%, 98%, 99%, or 100% sequence identity to an amino acid sequence selected from the group consisting of SEQ ID NOs: 28, 58, and 60-63;
an antibody comprising a complete H chain having at least 95% sequence identity to the amino acid sequence of SEQ ID NO: 74 or 79 and/or a complete L chain amino acid sequence having at least 90%, 91%, 92%, 93%, 94%, 95%, 96%, 97%, 98%, 99%, or 100% sequence identity to an amino acid sequence selected from the group consisting of SEQ ID NOs: 28, 58, 60-63, and 78;
an antibody comprising complete H/L chain sequences (respectively) comprising the amino acid sequences of SEQ ID NOs: 75/58 (h2A10.v2.T56A) or SEQ ID NOs: 79/78 (h2A10.v4);
an antibody that binds to the same epitope as an anti-TGFβ3 antibody comprising (a) HVR-H1 comprising the amino acid sequence of SEQ ID NO: 4; (b) HVR-H2 comprising the amino acid sequence of SEQ ID NO: 5; and (c) HVR-H3 comprising the amino acid sequence of SEQ ID NO:6;
an antibody that binds to the same epitope as an anti-TGFβ3 antibody comprising (a) HVR-H1 comprising the amino acid sequence of SEQ ID NO: 4; (b) HVR-H2 comprising the amino acid sequence of SEQ ID NO: 34; and (c) HVR-H3 comprising the amino acid sequence of SEQ ID NO:6;
an antibody that binds to the same epitope as an anti-TGFβ3 antibody comprising (a) HVR-H1 comprising the amino acid sequence of SEQ ID NO: 4; (b) HVR-H2 comprising the amino acid sequence of SEQ ID NO: 35; and (c) HVR-H3 comprising the amino acid sequence of SEQ ID NO:6;
an antibody that binds to the same epitope as an anti-TGFβ3 antibody comprising (a) HVR-H1 comprising the amino acid sequence of SEQ ID NO: 4; (b) HVR-H2 comprising the amino acid sequence of SEQ ID NO: 159; and (c) HVR-H3 comprising the amino acid sequence of SEQ ID NO:6;
an antibody that binds to the same epitope as an anti-TGFβ3 antibody comprising VH/VL sequences (respectively) selected from the group consisting of SEQ ID NOs: 53/36 (h2A10.v2.t56A), SEQ ID NOs: 57/56 (h2A10.v4), SEQ ID NOs: 51/36 (h2A10.v2.N54S), SEQ ID NOs: 55/54 (h2A10.v3), SEQ ID NOs: 52/36 (h2A10.v2.N54Q), SEQ ID NOs: 23/22 (rat 2A10), SEQ ID NOs: 37/36 (v1), SEQ ID NOs: 37/38 (v1.1), SEQ ID NOs: 37/39 (v1.2), SEQ ID NOs: 37/40 (v1.3), SEQ ID NOs: 37/41 (v1.4), SEQ ID NOs: 42/36 (v1.5), SEQ ID NOs: 43/36 (v1.6), SEQ ID NOs:44/36 (v1.7), SEQ ID NOs: 45/36 (v2), SEQ ID NOs: 45/38 (v2.1), SEQ ID NOs: 45/39 (v2.2), SEQ ID NOs: 45/40 (v2.3), SEQ ID NOs: 45/41 (v2.4), SEQ ID NOs: 46/36 (v2.5), SEQ ID NOs: 47/36 (v2.6), SEQ ID NOs: 48/36 (v2.7), SEQ ID NOs: 49/36 (v2.8), and SEQ ID NOs: 50/36 (v2.9);
an antibody that binds to the same epitope as an anti-TGFβ3 antibody comprising VH/VL sequences (respectively) selected from the group consisting of SEQ ID NOs: 53/36 (h2A10.v2.t56A), SEQ ID NOs: 57/56 (h2A10.v4), SEQ ID NOs: 51/36 (h2A10.v2.N54S), SEQ ID NOs: 55/54 (h2A10.v3), SEQ ID NOs: 52/36 (h2A10.v2.N54Q), SEQ ID NOs: 23/22 (rat 2A10), SEQ ID NOs: 37/36 (v1), SEQ ID NOs: 37/38 (v1.1), SEQ ID NOs: 37/39 (v1.2), SEQ ID NOs: 37/40 (v1.3), SEQ ID NOs: 37/41 (v1.4), SEQ ID NOs: 42/36 (v1.5), SEQ ID NOs: 43/36 (v1.6), SEQ ID NOs:44/36 (v1.7), SEQ ID NOs: 45/36 (v2), SEQ ID NOs: 45/38 (v2.1), SEQ ID NOs: 45/39 (v2.2), SEQ ID NOs: 45/40 (v2.3), SEQ ID NOs: 45/41 (v2.4), SEQ ID NOs: 46/36 (v2.5), SEQ ID NOs: 47/36 (v2.6), SEQ ID NOs: 48/36 (v2.7), SEQ ID NOs: 49/36 (v2.8), SEQ ID NOs: 50/36 (v2.9), SEQ ID NOs: 55/54 (v3), and SEQ ID NOs: 57/56 (v4);
an antibody that binds to the same epitope as an anti-TGFβ3 antibody comprising VH/VL sequences (respectively) of SEQ ID NOs: 57/56 (h2A10.v4);
an antibody that comprises an antigen binding domain that directly contacts amino acid residues R325, K331, W332, H334, E335, T387, I388, L389, Y391, V392, G393, R394, P396, K397, and V398 on human TGFβ3, wherein the anti-TGFβ3 antibody selectively neutralizes TGFβ3;
an antibody that selectively neutralizes TGFβ3 and has reduced toxicity in mice relative to the pan-TGFβ antibody 1D11;
an antibody that has reduced toxicity in mice relative to the pan-TGFβ antibody 1D11 at a dose of 50 mg/kg.

An isoform-selective anti-TGFβ antibody according to any of the above aspects of the methods of treating fibrosis may be a monoclonal antibody, including a chimeric, humanized or human antibody. In one embodiment, an isoform-selective anti-TGFβ antibody is an antibody fragment, e.g., a Fv, Fab, Fab′, scFv, diabody, or F(ab′)₂ fragment. In another embodiment, the antibody is a full length antibody, e.g., an intact IgG1 or IgG4 antibody, preferably an IgG1, or other antibody class or isotype as defined herein.

In some aspects of the provided methods for treating a TGFβ-related disorder, the method comprises administering to a subject having a TGFβ-related disorder an effective amount of an anti-TGFβ3 antibody comprising: heavy chain CDRs comprising CDR-H1, -H2 and -H3, wherein CDR-H1 has the amino acid sequence of SEQ ID NO: 4, CDR-H2 has the amino acid sequence of one of SEQ ID NOs: 5, 34, 35, and 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9. In some embodiments, the CDR-H2 has the amino acid sequence of SEQ ID NO: 35. In certain embodiments, the isolated anti-TGFβ3 antibody further comprises a VH having at least 90%, 91%, 92%, 93%, 94%, 95%, 96%, 97%, 98%, 99%, or 100% sequence identity to the amino acid sequence of SEQ ID NO: 57. In some embodiments, the anti-TGFβ3 antibody further comprises a VL having at least 90%, 91%, 92%, 93%, 94%, 95%, 96%, 97%, 98%, 99%, or 100% sequence identity to the amino acid sequence of SEQ ID NO: 56. In certain embodiments, the anti-TGFβ3 antibody further comprises a complete H chain having at least 90%, 91%, 92%, 93%, 94%, 95%, 96%, 97%, 98%, 99%, or 100% sequence identity to an amino acid sequence of SEQ ID NO: 79. In other embodiments, the anti-TGFβ3 antibody further comprises a complete L chain having at least 90%, 91%, 92%, 93%, 94%, 95%, 96%, 97%, 98%, 99%, or 100% sequence identity to an amino acid sequence of SEQ ID NO: 78. In some embodiments, the disorder is fibrosis. In certain embodiments, the disorder is SSc. In some embodiments, the disorder is IPF. In certain embodiments, the anti-TGFβ3 antibody is a monoclonal antibody, such as a chimeric, humanized or human antibody. In some embodiments, the anti-TGFβ3 antibody is an antibody fragment, e.g., a Fv, Fab, Fab′, scFv, diabody, or F(ab′)₂ fragment. In other embodiments, the anti-TGFβ3 antibody is a full length antibody, e.g., an intact IgG1 or IgG4 antibody, such as an IgG1, or other antibody class or isotype as defined herein.

### Medicaments and Uses of the Antibodies

As used herein, medicaments comprising an isoform-selective anti-TGFβ-antibody of the invention, manufacture of such medicaments, and uses of the medicaments and isoform-selective anti-TGFβ-antibodies of the invention for the treatment of a TGFβ-related disorder can include disruption of TGFβ-driven processes so as to halt progression of the TGFβ-related disorder, slow progression of the TGFβ-related disorder, or cause regression of the TGFβ-related disorder (i.e., improve the subject's (patient's) state of health with respect to the degree of the TGFβ-related disorder in the affected tissue or organ). In certain embodiments, where such uses and treatments precede onset of the TGFβ-related disorder, i.e., use or treatment is performed prior to a known or an otherwise expected onset of TGFβ-related disorder, then such use or treatment may include preventing development or onset of the TGFβ-related disorder. Administration of the various active agents can therefore be carried out for a suitable duration to either control or halt progression of the TGFβ-related disorder, or prevent onset thereof.

As used herein, medicaments comprising an isoform-selective anti-TGFβ-antibody of the invention, manufacture of such medicaments, and uses of the medicaments and isoform-selective anti-TGFβ-antibodies of the invention for the treatment of fibrosis or fibrotic conditions, includes disruption of the fibrotic processes so as to halt progression of the fibrotic condition, slow progression of the fibrotic condition, or cause regression of the fibrotic condition (i.e., improve the subject's (patient's) state of health with respect to the degree of fibrosis in the affected tissue or organ). In certain embodiments, where such uses and treatments precede onset of the fibrotic condition, i.e., the use or treatment is performed prior to a known or an otherwise expected onset of fibrosis, then such use or treatment may include preventing development or onset of the fibrotic condition. Administration of the various active agents can therefore be carried out for a suitable duration to either control or halt progression of the fibrotic condition, or prevent onset thereof.

Thus, in some aspects, the invention provides a medicament comprising an isoform-selective anti-TGFβ-antibody of the invention and/or uses of such medicament or antibody for treating fibrosis, wherein the fibrosis is a fibrotic condition of the lung, liver, heart, kidney, pancreas, eye and/or skin. In one aspect, the fibrosis is a lung fibrosis selected from idiopathic pulmonary fibrosis (IPF); idiopathic pulmonary upper lobe fibrosis (Amitani disease); familial pulmonary fibrosis; pulmonary fibrosis secondary to systemic inflammatory diseases such as, rheumatoid arthritis, scleroderma, lupus, cryptogenic fibrosing alveolitis, chronic obstructive pulmonary disease (COPD) or chronic asthma; cystic fibrosis; non-specific interstitial pneumonia (NSIP); cryptogenic organizing pneumonia (COP); progressive massive fibrosis, a complication of coal worker's pneumoconiosis; scleroderma/systemic sclerosis (SSc, including limited cutaneous (IcSSc) and diffuse cutaneous (dcSSc) forms and SSc-associated interstitial lung disease (SSc-ILD)); bronchiolitis obliterans-organizing pneumonia; connective tissue disease-associated ILD (CT-ILD), progressive fibrosing ILD, hypersensitivity pneumonitis, pulmonary hypertension; pulmonary tuberculosis; silicosis; asbestosis; acute lung injury; and acute respiratory distress (ARD; including bacterial pneumonia induced, trauma-induced, and viral pneumonia-induced, ventilator-induced, non-pulmonary sepsis induced). In another aspect, the fibrosis is a fibrotic condition of the liver selected from liver cirrhosis due to all etiologies; congenital hepatic fibrosis; obesity; fatty liver; alcohol induced liver fibrosis; non-alcoholic steatohepatitis (NASH); biliary duct injury; primary biliary cirrhosis (PBC), primary sclerosing cholangitis (PSC); infection- or viral-induced liver fibrosis (e.g., chronic hepatitis B and C virus infections); cystic fibrosis; autoimmune hepatitis; necrotizing hepatitis; primary sclerosing cholangitis; hemochromatosis; disorders of the biliary tree; hepatic dysfunction attributable to infections. In another aspect, the fibrosis is a fibrotic condition of the heart and/or pericardium (i.e., heart or pericardial fibrosis, or fibrosis of the associate vasculature) selected from endomyocardial fibrosis; cardiac allograft vasculopathy (CAV); myocardial infarction; atrial fibrosis; congestive heart failure; arteriosclerosis; atherosclerosis; vascular stenosis; myocarditis; congestive cardiomyopathy; coronary infarcts; varicose veins; coronary artery stenosis and other post-ischemic conditions; and idiopathic retroperitoneal fibrosis. In another aspect, the fibrosis is a fibrotic condition of the kidney selected from glomerulonephritis (including membranoproliferative, diffuse proliferative, rapidly progressive or sclerosing, post-infectious and chronic forms); diabetic glomerulosclerosis; focal segmental glomerulosclerosis; IgA nephropathy; diabetic nephropathy; ischemic nephropathy, tubulointerstitial kidney fibrosis, HIV-associated nephropathy; membrane nephropathy; glomerulonephritis secondary to systemic inflammatory diseases such as lupus, scleroderma and diabetes glomerulonephritis; idiopathic membranoproliferative glomerular nephritis; mesangial proliferative glomerulonephritis; crescentic glomerulonephritis; amyloidosis (which affects the kidney among other tissues); autoimmune nephritis; renal tubulointerstitial fibrosis; renal arteriosclerosis; Alport's syndrome; nephrotic syndrome; chronic renal failure; chronic kidney disease, periglomerular fibrosis/atubular glomeruli; combined apical emphysema and basal fibrosis syndrome (emphysema/fibrosis syndrome); glomerular hypertension; nephrogenic fibrosing dermatopathy; polycystic kidney disease; Fabry's disease and renal hypertension. In another aspect, the fibrosis is a fibrotic condition of the pancreas selected from stromal remodeling pancreatitis and stromal fibrosis. In another aspect, the fibrosis is a fibrotic condition of the gastrointestinal tract (i.e., GI tract fibrosis) selected from Crohn's disease; ulcerative colitis; collagenous colitis; colorectal fibrosis; villous atrophy; crypt hyperplasia; polyp formation; healing gastric ulcer; and microscopic colitis. In another aspect, the fibrosis is a fibrotic condition of the eye selected from ocular fibrosis, ophthalmic fibrosis, proliferative vitreoretinopathy; vitreoretinopathy of any etiology; fibrosis associated with retinal dysfunction; fibrosis associated with wet or dry macular degeneration; scarring in the cornea and conjunctiva; fibrosis in the corneal endothelium; anterior subcapsular cataract and posterior capsule opacification; anterior segment fibrotic diseases of the eye; fibrosis of the corneal stroma (e.g., associated with corneal opacification); fibrosis of the trabecular network (e.g., associated with glaucoma); posterior segment fibrotic diseases of the eye; fibrovascular scarring (e.g., in retinal or choroidal vasculature of the eye); retinal fibrosis; epiretinal fibrosis; retinal gliosis; subretinal fibrosis (e.g., associated with age related macular degeneration); tractional retinal detachment in association with contraction of the tissue in diabetic retinopathy; congenital orbital fibrosis; lacrimal gland fibrosis; corneal subepithelial fibrosis; and Grave's ophthalmopathy. In another aspect, the fibrosis is a selected from fibrosis resulting from spinal cord injury/fibrosis or central nervous system fibrosis such as fibrosis after a stroke, Duchenne muscular dystrophy, dupuytren's contracture, fibrosis associated with neurodegenerative disorder such as Alzheimer's disease or multiple sclerosis; vascular restenosis; uterine fibrosis; endometriosis; ovarian fibroids; Peyronie's disease; polycystic ovarian syndrome; disease related pulmonary apical fibrosis in ankylosing spondylitis; scarring; and fibrosis incident to microbial infections (e.g., bacterial, viral, parasitic, fungal etc.).

In one embodiment, the invention provides a medicament comprising an isoform-selective anti-TGFβ-antibody of the invention and/or uses of such medicament or antibody for treating a TGFβ-related disorder, wherein the use includes administering to a subject having such TGFβ-related disorder an effective amount of an isoform-selective anti-TGFβ antibody (e.g., anti-TGFβ2, anti-TGFβ2/3, or anti-TGFβ3 antibody). In one such embodiment, the use further comprises administering to the subject an effective amount of at least one additional therapeutic agent, as described below.

In one embodiment, the invention provides a medicament comprising an isoform-selective anti-TGFβ-antibody of the invention and/or uses of such medicament or antibody for treating fibrosis (e.g., a fibrotic condition as described above), wherein the use includes administering to a subject having such fibrosis an effective amount of an isoform-selective anti-TGFβ antibody (e.g., anti-TGFβ2, anti-TGFβ2/3, or anti-TGFβ3 antibody). In one such embodiment, the use further comprises administering to the subject an effective amount of at least one additional therapeutic agent, as described below.

In a further aspect, an isoform-selective anti-TGFβ antibody as described herein (e.g., anti-TGFβ2, anti-TGFβ2/3, or anti-TGFβ3 antibody) for use as a medicament is provided. In further aspects, an isoform-selective anti-TGFβ antibody (e.g., anti-TGFβ2, anti-TGFβ2/3, or anti-TGFβ3 antibody) for use in treating a TGFβ-related disorder (e.g., fibrosis, e.g., fibrosis of the lung, e.g., IPF, liver, pancreas, heart, kidney, eye, and/or skin, e.g., SSc) is provided. In certain embodiments, an isoform-selective anti-TGFβ antibody (e.g., anti-TGFβ2, anti-TGFβ2/3, or anti-TGFβ3 antibody) for use in a method of treatment is provided. In certain embodiments, the invention provides an isoform-selective anti-TGFβ antibody (e.g., anti-TGFβ2, anti-TGFβ2/3, or anti-TGFβ3 antibody) for use in a method of treating a subject having fibrosis comprising administering to the individual an effective amount of the isoform-selective anti-TGFβ antibody (e.g., anti-TGFβ2, anti-TGFβ2/3, or anti-TGFβ3 antibody). In one such embodiment, the use further comprises administering to the individual an effective amount of at least one additional therapeutic agent, e.g., as described below. In further embodiments, the invention provides an isoform-selective anti-TGFβ antibody (e.g., anti-TGFβ2, anti-TGFβ2/3, or anti-TGFβ3 antibody) for use in inhibiting TGFBR-dependent SMAD signaling, inhibiting the assembly of TGFβ-TGFBR signaling complexes, inhibiting TGFβ signaling through the TGFBR1/R2 complex, inhibiting TGFβ signaling through the TGFBR2/ALK1 complex facilitated by endoglin and/or for inhibiting new collagen synthesis in a subject. In certain embodiments, the invention provides an isoform-selective anti-TGFβ antibody (e.g., anti-TGFβ2, anti-TGFβ2/3, or anti-TGFβ3 antibody) for use in a method of inhibiting TGFBR-dependent SMAD signaling, inhibiting the assembly of TGFβ-TGFBR signaling complexes, inhibiting TGFβ signaling through the TGFBR1/R2 complex, inhibiting TGFβ signaling through the TGFBR2/ALK1 complex facilitated by endoglin and/or for inhibiting new collagen synthesis in a subject comprising administering to the subject an effective amount of the isoform-selective anti-TGFβ antibody (e.g., anti-TGFβ2, anti-TGFβ2/3, or anti-TGFβ3 antibody) to inhibit TGFBR-dependent SMAD signaling, to inhibit the assembly of TGFβ-TGFBR signaling complexes, to inhibit TGFβ signaling through the TGFBR1/R2 complex, to inhibit TGFβ signaling through the TGFBR2/ALK1 complex facilitated by endoglin, and/or for inhibiting new collagen synthesis in the subject. A subject according to any of the above embodiments is preferably a human.

In a further aspect, the invention provides for the use of an isoform-selective anti-TGFβ antibody (e.g., anti-TGFβ2, anti-TGFβ2/3, or anti-TGFβ3 antibody) in the manufacture or preparation of a medicament. In one embodiment, the medicament is for treatment of a TGFβ-related disorder, e.g., fibrosis, such as but not limited to fibrosis, e.g., fibrosis of the lung, e.g., IPF, liver, pancreas, heart, kidney, eye, and/or skin, e.g., SSc. In a further embodiment, the medicament is for use in a method of treating fibrosis comprising administering to a subject having fibrosis an effective amount of the medicament. In one such embodiment, the use further comprises administering to the subject an effective amount of at least one additional therapeutic agent, e.g., as described below. In a further embodiment, the medicament is for inhibiting TGFBR-dependent SMAD signaling, inhibiting the assembly of TGFβ-TGFBR signaling complexes, inhibiting TGFβ signaling through the TGFBR1/R2 complex, inhibiting TGFβ signaling through the TGFBR2/ALK1 complex facilitated by endoglin, and/or for inhibiting new collagen synthesis in a subject. In a further embodiment, the medicament is for use in a method of inhibiting TGFBR-dependent SMAD signaling, inhibiting the assembly of TGFβ-TGFBR signaling complexes, inhibiting TGFβ signaling through the TGFBR1/R2 complex, inhibiting TGFβ signaling through the TGFBR2/ALK1 complex facilitated by endoglin, and/or for inhibiting new collagen synthesis in a subject, comprising administering to the individual an effective amount of the medicament to inhibit TGFBR-dependent SMAD signaling, to inhibit the assembly of TGFβ-TGFBR signaling complexes, to inhibit TGFβ signaling through the TGFBR1/R2 complex, to inhibit TGFβ signaling through the TGFBR2/ALK1 complex facilitated by endoglin, and/or to inhibit new collagen synthesis in the subject.

### Anti-TGFβ2 Antibodies

In any of the above aspects, with respect to the isoform-selective anti-TGFβ antibodies as described herein (e.g., anti-TGFβ2, anti-TGFβ2/3, or anti-TGFβ3 antibody) as a medicament (and manufacture or preparation or use thereof) and/or for use in a method of treatment, and/or for inhibiting TGFBR-dependent SMAD signaling, for inhibiting the assembly of TGFβ-TGFBR signaling complexes, for inhibiting TGFβ signaling through the TGFBR1/R2 complex, for inhibiting TGFβ signaling through the TGFBR2/ALK1 complex facilitated by endoglin, and/or for inhibiting new collagen synthesis in a subject, e.g., for treating fibrosis (e.g., fibrosis of the lung, e.g., IPF, liver, pancreas, heart, kidney, eye, and/or skin, e.g., SSc), an anti-TGFβ2 antibody of the invention can have one or more of the following properties:
the anti-TGFβ2 antibody:
(a) selectively neutralizes TGFβ2;
(b) has reduced toxicity relative to the pan-TGFβ antibody 1D11;
(c) has reduced toxicity relative to the pan-TGFβ antibody 1D11;
(d) has reduced toxicity in rodents relative to the pan-TGFβ antibody 1D11;
(e) has reduced toxicity relative to the pan-TGFβ small molecule inhibitor galunisertib; and/or
(f) has reduced toxicity in rodents relative to the pan-TGFβ small molecule inhibitor galunisertib; and
wherein in some embodiments, the use, medicament, or method is for treating fibrosis and wherein the fibrosis is SSc or IPF.

In further aspects, with respect to the isoform-selective anti-TGFβ antibodies as described herein (e.g., anti-TGFβ2, anti-TGFβ2/3, or anti-TGFβ3 antibody) as a medicament (and manufacture or preparation or use thereof) and/or for use in a method of treatment, and/or for inhibiting TGFBR-dependent SMAD signaling, for inhibiting the assembly of TGFβ-TGFBR signaling complexes, for inhibiting TGFβ signaling through the TGFBR1/R2 complex, for inhibiting TGFβ signaling through the TGFBR2/ALK1 complex facilitated by endoglin in a subject, and/or for inhibiting new collagen synthesis e.g., for treating fibrosis (e.g., fibrosis of the lung, liver, pancreas, heart, kidney, eye, and/or skin), an anti-TGFβ2 antibody of the invention can have one or more of the following properties:
the anti-TGFβ2 antibody selectively neutralizes TGFβ2; the anti-TGFβ2 binds to TGFβ2 with a K_{D} of less than 10 pM and/or has a cell-based IC₅₀ of less than 250 pM; binds to TGFβ2 with a K_{D} of less than or equal to about 5 pM, about 4 pM, about 3 pM, about 2 pM, or about 1 pM; the anti-TGFβ2 antibody provided herein has a cell-based IC₅₀ less than or equal to about 250 pM, about 200 pM, about 150 pM, about 100 pM, about 75 pM, about 50 pM, or about 40 pM.

In any of the above aspects, with respect to the isoform-selective anti-TGFβ antibodies as described herein (e.g., anti-TGFβ2, anti-TGFβ2/3, or anti-TGFβ3 antibody) as a medicament (and manufacture or preparation or use thereof) and/or for use in a method of treatment, and/or for inhibiting TGFBR-dependent SMAD signaling, for inhibiting the assembly of TGFβ-TGFBR signaling complexes, for inhibiting TGFβ signaling through the TGFBR1/R2 complex, for inhibiting TGFβ signaling through the TGFBR2/ALK1 complex facilitated by endoglin in a subject, and/or for inhibiting new collagen synthesis e.g., for treating fibrosis (e.g., fibrosis of the lung, liver, pancreas, heart, kidney, eye, and/or skin), an anti-TGFβs antibody of the invention can have one or more of the following properties:
an anti-TGFβ2 antibody comprising at least one, two, three, four, five, or six HVRs selected from (a) HVR-H1 comprising the amino acid sequence of SEQ ID NO: 16; (b) HVR-H2 comprising the amino acid sequence of SEQ ID NO:17; (c) HVR-H3 comprising the amino acid sequence of SEQ ID NO: 18; (d) HVR-L1 comprising the amino acid sequence of SEQ ID NO: 19; (e) HVR-L2 comprising the amino acid sequence of SEQ ID NO: 20; and (f) HVR-L3 comprising the amino acid sequence of SEQ ID NO: 21;
an anti-TGFβ2 antibody comprising at least one, at least two, or all three VH HVR sequences selected from (a) HVR-H1 comprising the amino acid sequence of SEQ ID NO: 16; (b) HVR-H2 comprising the amino acid sequence of SEQ ID NO: 17; (c) HVR-H3 comprising the amino acid sequence of SEQ ID NO: 18. In another aspect, the invention provides an antibody comprising (a) HVR-H1 comprising the amino acid sequence of SEQ ID NO: 16; (b) HVR-H2 comprising the amino acid sequence of SEQ ID NO:17; ad (c) HVR-H3 comprising the amino acid sequence of SEQ ID NO: 18;
an anti-TGFβ2 antibody comprising at least one, at least two, or all three VL HVR sequences selected from (a) HVR-L1 comprising the amino acid sequence of SEQ ID NO: 19; (b) HVR-L2 comprising the amino acid sequence of SEQ ID NO: 20; and (c) HVR-L3 comprising the amino acid sequence of SEQ ID NO: 21. In one embodiment, the antibody comprises (a) HVR-L1 comprising the amino acid sequence of SEQ ID NO: 19; (b) HVR-L2 comprising the amino acid sequence of SEQ ID NO: 20; and (c) HVR-L3 comprising the amino acid sequence of SEQ ID NO: 21;
an anti-TGFβ2 antibody comprising HVR-H3 comprising the amino acid sequence of SEQ ID NO: 18 and HVR-L3 comprising the amino acid sequence of SEQ ID NO: 21. In a further embodiment, the anti-TGFβ2 antibody comprises HVR-H3 comprising the amino acid sequence of SEQ ID NO: 18, HVR-L3 comprising the amino acid sequence of SEQ ID NO: 21, and HVR-H2 comprising the amino acid sequence of SEQ ID NO: 17;
an anti-TGFβ2 antibody comprising (a) a VH domain comprising at least one, at least two, or all three VH HVR sequences selected from (i) HVR-H1 comprising the amino acid sequence of SEQ ID NO: 16; (ii) HVR-H2 comprising the amino acid sequence of SEQ ID NO: 17; (iii) HVR-H3 comprising the amino acid sequence of SEQ ID NO: 18; and (b) a VL domain comprising at least one, at least two, or all three VL HVR sequences selected from (i) HVR-L1 comprising the amino acid sequence of SEQ ID NO: 19, (ii) HVR-L2 comprising the amino acid sequence of SEQ ID NO: 20, and (c) HVR-L3 comprising the amino acid sequence of SEQ ID NO: 21; and
an anti-TGFβ2 antibody comprising a) HVR-H1 comprising the amino acid sequence of SEQ ID NO: 16; (b) HVR-H2 comprising the amino acid sequence of SEQ ID NO:17; (c) HVR-H3 comprising the amino acid sequence of SEQ ID NO: 18; (d) HVR-L1 comprising the amino acid sequence of SEQ ID NO: 19; (e) HVR-L2 comprising the amino acid sequence of SEQ ID NO: 20; and (f) HVR-L3 comprising the amino acid sequence of SEQ ID NO: 21.

In any of the above aspects, the isoform-selective anti-TGFβ may be humanized. In one embodiment, the isoform-selective anti-TGFβ antibody comprises HVRs as in any of the above embodiments, and further comprises an acceptor human framework, e.g. a human immunoglobulin framework or a human consensus framework. In any of the above embodiments, a humanized anti-TGFβ2 antibody comprises one or more mutations in the VH framework selected from the group consisting of 37V or 37I, 48M or 48L, 49G or 49A, 67L, 71K and 78V, and 105P or 105R. In some embodiments, the anti-TGFβ2 antibody comprises a VH, wherein the VH comprises the amino acid sequence of SEQ ID NO: 25, wherein the VH comprises a set of framework mutations selected from the group consisting of: (i) 37V, 48M and 49G in FR2 and 105P in FR4 (h6F12.v1 and h6F12.v3); (ii) 37V and 48M in FR2, 67L, 71K and 78V in FR3 and 105P in FR4 (h6F12.v2 and h6F12.v4); (iii) 37I in FR2 (h6F12.v1.6); (iv) 48L in FR2 (h6F12.v1.7); (v) 49A in FR2 (h6F12.v1.8); (vi) 105R in FR4 (h6F12.v1.9); (vii) 37V, 48M and 49G in FR2 and 105P in FR4 (h6F12.v1 and h6F12.v3); (viii) 37V and 48M in FR2, 67L, 71K and 78V in FR3 and 105P in FR4 (6F12.v2 and h6F12.v4); (ix) 37I in FR2 (h6F12.v1.6); (x) 48L in FR2 (h6F12.v1.7); (xi): 49A in FR2 (h6F12.v1.8); and (xii) 105R in FR4 (h6F12.v1.9). In any of the above embodiments, a humanized anti-TGFβ2 antibody comprises one or more mutations in the VL framework selected from the group consisting of 43S or 43A, 66G, 69T, 71F, and 87Y relative to the VL having the amino acid sequence of SEQ ID NO: 24. In some embodiments, the anti-TGFβ2 antibody comprises a VL, wherein the VL comprises the amino acid sequence of SEQ ID NO: 24, wherein the VL comprises a set of framework mutations selected from the group consisting of: (i) 43S in FR2 and 66E, 69P, 71Y and 87F in FR3 (h6F12.v1 and h6F12.v2); (ii) 43S in FR2 and 58V, 66E, 69P, 71Y and 87F in FR3 (h6F12.v3 and h6F12.v4); (iii) 43A in FR2 (h6F12.v1.1); (iv) 66G in FR3 (h6F12.v1.2); (v) 69T in FR3 (h6F12.v1.3); (vi) 71F in FR3 (h6F12.v1.4); and (vii) 87Y in FR3 (h6F12.v1.5).

In any of the above aspects, with respect to the isoform-selective anti-TGFβ antibodies as described herein (e.g., anti-TGFβ2, anti-TGFβ2/3, or anti-TGFβ3 antibody) as a medicament (and manufacture or preparation or use thereof) and/or for use in a method of treatment, and/or for inhibiting TGFBR-dependent SMAD signaling, for inhibiting the assembly of TGFβ-TGFBR signaling complexes, for inhibiting TGFβ signaling through the TGFBR1/R2 complex, for inhibiting TGFβ signaling through the TGFBR2/ALK1 complex facilitated by endoglin in a subject, and/or for inhibiting new collagen synthesis e.g., for treating fibrosis (e.g., fibrosis of the lung, liver, pancreas, heart, kidney, eye, and/or skin), an isoform-selective anti-TGFβ antibody of the invention can have one or more of the following properties:
an anti-TGFβ2 antibody comprising a heavy chain variable domain (VH) sequence having at least 90%, 91%, 92%, 93%, 94%, 95%, 96%, 97%, 98%, 99%, or 100% sequence identity to the amino acid sequence selected from the group consisting of SEQ ID NO: 25, 132, and 138-142; or the VH comprises one, two or three HVRs selected from: (a) HVR-H1 comprising the amino acid sequence of SEQ ID NO: 16; (b) HVR-H2 comprising the amino acid sequence of SEQ ID NO:17; (c) HVR-H3 comprising the amino acid sequence of SEQ ID NO: 18; and the antibody comprises a light chain variable domain (VL) having at least 90%, 91%, 92%, 93%, 94%, 95%, 96%, 97%, 98%, 99%, or 100% sequence identity to the amino acid sequence selected from the group consisting of SEQ ID NO: 24, 131, 133-137, 143, and 144, or the VL comprises one, two or three HVRs selected from (a) HVR-L1 comprising the amino acid sequence of SEQ ID NO: 19; (b) HVR-L2 comprising the amino acid sequence of SEQ ID NO: 20; and (c) HVR-L3 comprising the amino acid sequence of SEQ ID NO: 21;
an anti-TGFβ2 antibody comprising the VH/VL sequences (respectively) selected from the group consisting of SEQ ID NOs: 25/24 (rabbit 6F12), SEQ ID NOs: 132/131 (v1), SEQ ID NOs: 132/133 (v1.1), SEQ ID NOs: 132/134 (v1.2), SEQ ID NOs: 132/135 (v1.3), SEQ ID NOs: 132/136 (v1.4), SEQ ID NOs: 132/137 (v1.5), SEQ ID NOs: 138/131 (v1.6), SEQ ID NOs: 139/131 (v1.7), SEQ ID NOs: 140/131 (v1.8), SEQ ID NOs: 141/131 (v1.9), SEQ ID NOs: 142/131 (v2), SEQ ID NOs: 132/143 (v3), and SEQ ID NOs: 142/144 (v4); or
an anti-TGFβ2 antibody comprising a complete H chain amino acid sequence having at least 95% sequence identity to the amino acid sequence selected from the group consisting of SEQ ID NOs: 31, 146, and 152-156 and/or a complete L chain amino acid sequence having an amino acid sequence selected from the group consisting of SEQ ID NOs: 30, 145, 147-151, 157, and 158;
an anti-TGFβ2 antibody comprising a complete H/L chain pair, the complete H/L chain pair (respectively) comprising amino acid sequences selected from the group consisting of SEQ ID NOs: 31/30 (rabbit 6F12), SEQ ID NOs: 146/145 (v1), SEQ ID NOs: 146/147 (v1.1), SEQ ID NOs: 146/148 (v1.2), SEQ ID NOs: 146/149 (v1.3), SEQ ID NOs: 146/150 (v1.4), SEQ ID NOs: 146/151 (v1.5), SEQ ID NOs: 152/145 (v1.6), SEQ ID NOs: 153/145 (v1.7), SEQ ID NOs: 154/145 (v1.8), SEQ ID NOs: 155/145 (v1.9), SEQ ID NOs: 156/145 (v2), SEQ ID NOs: 146/157 (v3), and SEQ ID NOs: 156/158 (v4);
an antibody that binds to the same epitope as an anti-TGFβ2 antibody comprising a) HVR-H1 comprising the amino acid sequence of SEQ ID NO: 16; (b) HVR-H2 comprising the amino acid sequence of SEQ ID NO:17; (c) HVR-H3 comprising the amino acid sequence of SEQ ID NO: 18; (d) HVR-L1 comprising the amino acid sequence of SEQ ID NO: 19; (e) HVR-L2 comprising the amino acid sequence of SEQ ID NO: 20; and (f) HVR-L3 comprising the amino acid sequence of SEQ ID NO: 21.

### Anti-TGFβ2/3 Antibodies

In any of the above aspects, with respect to the isoform-selective anti-TGFβ antibodies as described herein (e.g., anti-TGFβ2, anti-TGFβ2/3, or anti-TGFβ3 antibody) as a medicament (and manufacture or preparation or use thereof) and/or for use in a method of treatment, and/or for inhibiting TGFBR-dependent SMAD signaling, for inhibiting the assembly of TGFβ-TGFBR signaling complexes, for inhibiting TGFβ signaling through the TGFBR1/R2 complex, for inhibiting TGFβ signaling through the TGFBR2/ALK1 complex facilitated by endoglin in a subject, and/or for inhibiting new collagen synthesis e.g., for treating fibrosis (e.g., fibrosis of the lung, liver, pancreas, heart, kidney, eye, and/or skin), an anti-TGFβ2/3 antibody that selectively neutralizes TGFβ2 and TGFβ3 as described herein can have one or more of the following properties:
(a) selectivity of the anti-TGFβ2/3 antibody for TGFβ2 and TGFβ3 over human TGFβ1, with respect to selective neutralization, is achieved by direct contact of the antibody's antigen binding domain with amino acid residue E373 of TGFβ2 or TGFβ3 (human TGFβ2 numbering);
(b) the anti-TGFβ2/3 antibody neutralizes TGFβ2 and/or TGFβ3 via an allosteric mechanism;
(c) the anti-TGFβ2/3 antibody induces a conformational change in TGFβ2 homodimer;
(d) the anti-TGFβ2/3 antibody induces a conformational change in TGFβ2 homodimer, wherein the conformational change comprises the two monomers pinching together by several degrees;
(e) the anti-TGFβ2/3 antibody is a divalent antibody or a monovalent antibody;
(f) the anti-TGFβ2/3 antibody comprises (i) heavy chain CDRs comprising CDR-H1, -H2 and -H3, wherein CDR-H1 has the amino acid sequence of SEQ ID NO: 10, CDR-H2 has the amino acid sequence of SEQ ID NO: 11, and CDR-H3 has the amino acid sequence of SEQ ID NO: 12; and (ii) light chain CDRs comprising CDR-L1, -L2 and -L3, wherein CDR-L1 has the amino acid sequence of SEQ ID NO: 13; CDR-L2 has the amino acid sequence of SEQ ID NO: 14, and CDR-L3 has the amino acid sequence of SEQ ID NO: 15;
(g) the anti-TGFβ2/3 antibody binds to TGFβ2 homodimer, the TGFβ2 homodimer having a first and a second TGFβ2 monomer, and wherein the anti-TGFβ2/3 antibody comprises an antigen binding domain that directly contacts (i) amino acid residues V313, Q314, D315, R320, L322, Y323, R328, D329, F345, and A347 of the first TGFβ2 monomer, and (ii) amino acid residues N368, T369, I370, N371, P372, E373, A374, S375, A376, and S377 of the second TGFβ2 monomer (human TGFβ2 numbering);
(h) the anti-TGFβ2/3 antibody as in (g), wherein the antigen binding domain is within 5 angstroms of the TGFβ2 and/or TGFβ3 amino acid residues;
(i) wherein the anti-TGFβ2/3 antibody binds to a substantially similar epitope as in (g) in TGFβ3; and
(j) the anti-TGFβ2/3 antibody does not neutralize TGFβ2 and/or TGFβ3 in single arm form; and
wherein in some embodiments, the use, medicament, or method is for treating fibrosis and wherein the fibrosis is SSc or IPF.

In any of the above aspects, with respect to the isoform-selective anti-TGFβ antibodies as described herein (e.g., anti-TGFβ2, anti-TGFβ2/3, or anti-TGFβ3 antibody) as a medicament (and manufacture or preparation or use thereof) and/or for use in a method of treatment, and/or for inhibiting TGFBR-dependent SMAD signaling, for inhibiting the assembly of TGFβ-TGFBR signaling complexes, for inhibiting TGFβ signaling through the TGFBR1/R2 complex, for inhibiting TGFβ signaling through the TGFBR2/ALK1 complex facilitated by endoglin in a subject, and/or for inhibiting new collagen synthesis e.g., for treating fibrosis (e.g., fibrosis of the lung, liver, pancreas, heart, kidney, eye, and/or skin), an anti-TGFβ2/3 antibody of the invention can have one or more of the following properties:
selectively neutralizes TGFβ2 and TGFβ3; binds to TGFβ2 and/or TGFβ3 with a K_{D} of less than 10 pM and/or has a cell-based IC₅₀ less than 250 pM;
binds to TGFβ2 and/or TGFβ3 with a K_{D} of less than about 10 pM, about 9 pM, about 8 pM, about 7 pM, about 6 pM, about 5 pM, about 4 pM, about 3 pM, about 2 pM, or about 1 pM;
has a cell-based IC₅₀ for inhibition of TGFβ2 of about 250 pM, about 200 pM, about 150 pM, about 100 pM, about 75 pM, or about 50 pM;
has a cell-based IC₅₀ for inhibition of TGFβ3 of about 250 pM, about 200 pM, about 150 pM, about 100 pM, about 75 pM, about 50 pM, about 40 pM, about 30 pM or less than about 30 pM; or
has a cell-based IC₅₀ for inhibition of TGFβ2 of about 250 pM and a cell-based IC₅₀ for inhibition (neutralization) of TGFβ3 of about 30 pM.

In any of the above aspects, with respect to the use of an isoform-selective anti-TGFβ antibody as described herein (e.g., anti-TGFβ2, anti-TGFβ2/3, or anti-TGFβ3 antibody) as a medicament (and manufacture or preparation thereof) and/or for use in a method of treatment, e.g., for treating fibrosis (e.g., fibrosis of the lung, liver, pancreas, heart, kidney, eye, and/or skin), the isoform-selective anti-TGFβ antibody can be selected from:
an anti-TGFβ2/3 antibody comprising at least one, two, three, four, five, or six HVRs selected from (a) HVR-H1 comprising the amino acid sequence of SEQ ID NO: 10; (b) HVR-H2 comprising the amino acid anti-TGFβ2/3 antibody sequence of SEQ ID NO: 11; (c) HVR-H3 comprising the amino acid sequence of SEQ ID NO: 12; (d) HVR-L1 comprising the amino acid sequence of SEQ ID NO: 13; (e) HVR-L2 comprising the amino acid sequence of SEQ ID NO: 14; and (f) HVR-L3 comprising the amino acid sequence of SEQ ID NO: 15;
an anti-TGFβ2/3 antibody comprising at least one, at least two, or all three VH HVR sequences selected from (a) HVR-H1 comprising the amino acid sequence of SEQ ID NO: 10; (b) HVR-H2 comprising the amino acid anti-TGFβ2/3 antibody sequence of SEQ ID NO: 11; (c) HVR-H3 comprising the amino acid sequence of SEQ ID NO: 12, and at least one, at least two, or all three VL HVR sequences selected from (a) HVR-L1 comprising the amino acid sequence of SEQ ID NO: 13; (b) HVR-L2 comprising the amino acid sequence of SEQ ID NO: 14; and (c) HVR-L3 comprising the amino acid sequence of SEQ ID NO: 15;
an anti-TGFβ2/3 antibody comprising (a) HVR-H1 comprising the amino acid sequence of SEQ ID NO: 10; (b) HVR-H2 comprising the amino acid sequence of SEQ ID NO: 11; (c) HVR-H3 comprising the amino acid sequence of SEQ ID NO: 12; (d) HVR-L1 comprising the amino acid sequence of SEQ ID NO: 13; (e) HVR-L2 comprising the amino acid sequence of SEQ ID NO: 14; and (f) HVR-L3 comprising an amino acid sequence selected from SEQ ID NO: 15.

In any of the above uses, the anti-TGFβ2/3 antibody may be humanized. In one embodiment, an anti-TGFβ2/3 antibody comprises HVRs as in any of the above embodiments, and further comprises an acceptor human framework, e.g. a human immunoglobulin framework or a human consensus framework. In some embodiments, the anti-TGFβ2/3 antibody comprises a VH, wherein the VH comprises the amino acid sequence of SEQ ID NO: 27, wherein the VH comprises a set of framework modifications selected from the group consisting of: (i) 2Q and 24V in FR1, 48I and 49G in FR2, 71K, 73S, 78V and 91F in FR3 and 105P in FR4 (h4A11.v, h4A11.v2, h4A11.v5, h4A11.v6); (ii) 2Q in FR1, 37V in FR2, 67F, 71K, 73 S, 78V and 91F in FR3 and 105P in FR4 (h4A11.v3, h4A11.v4, 4A11.v7, h4A11.v8); (iii) delete 1E in FR1 (h4A11.v7.1); (iv) delete 75K and 76N in FR3 (h4A11.v7.2); (v) delete 1E in FR1 and 75K76N in FR3 (h4A11.v7.3); (vi) 2V in FR1 (h4A11.v7.8); (vi) 37I in FR2 (h4A11.v7.9); (vii) 67V in FR3 (h4A11.v7.10); (viii) 71V in FR3 (h4A11.v7.11); (ix) 73T in FR3 (h4A11.v7.12); (x) 78F in FR3 (h4A11.v7.13); (xi) 91Y in FR3 (h4A11.v7.14); (xii) 105Q in FR4 (h4A11.v7.15); (xiii) 2V in FR1, 37I in FR2, 67V, 73T, 78F in FR3, 105Q in FR4 ((h4A11.v7.16); (xiv) 2V in FR1, 37I in FR2, 67V, 73T, 91Y in FR3, 105Q in FR4 (h4A11.v7.17); (xv) 2V in FR1, 37I in FR2, 67V, 73T in FR3, 105Q in FR4 (h4A11.v7.18); and (xvi) 2V in FR1, 37I in FR2, 67V, 73T, deletion of 75K and 76N in FR3, 105Q in FR4 (h4A11.v7.19. In another embodiment, an anti-TGFβ2/3 antibody comprises HVRs as in any of the above embodiments, and further comprises a VL comprising FR modifications selected from the group consisting of: 2A or 2I, 4L, 36F or 36Y, 43P or 43A, and 58V or 58I, relative to the VL having the amino acid sequence of SEQ ID NO: 26. In some embodiments, the anti-TGFβ2/3 antibody comprises a VL, wherein the VL comprises the amino acid sequence of SEQ ID NO: 26, wherein the VL comprises a set of framework modifications selected from the group consisting of: (i) 2A and 4L in FR1 and 36F in FR2 (h4A11v1 and h4A11.v3); (ii) 2A and 4L in FR1 and 36F and 43P in FR2 (h4A11.v2 and h4A11.v4); (iii) 2A in FR1, 36F and 43P in FR2 and 58V in FR3 (h4A11.v5 and h4A11.v7); (iv) 2A and 4L in FR1 and 36F in FR2 (h4A11.v6 and h4A11.v8); (v) 2I in FR1 (h4A11.v7.4); (vi) 36Y in FR2 (h4A11.v7.5); (vii) 43A in FR2 (h4A11.v7.6); (viii) 58I in FR3 (h4A11.v7.7); and (ix) 2I in FR1, 43A in FR2, 58I in FR3 (h4A11.v7.16-19).

In any of the above aspects, with respect to the isoform-selective anti-TGFβ antibodies as described herein (e.g., anti-TGFβ2, anti-TGFβ2/3, or anti-TGFβ3 antibody) as a medicament (and manufacture or preparation or use thereof) and/or for use in a method of treatment, and/or for inhibiting TGFBR-dependent SMAD signaling, for inhibiting the assembly of TGFβ-TGFBR signaling complexes, for inhibiting TGFβ signaling through the TGFBR1/R2 complex, for inhibiting TGFβ signaling through the TGFBR2/ALK1 complex facilitated by endoglin in a subject, and/or for inhibiting new collagen synthesis e.g., for treating fibrosis (e.g., fibrosis of the lung, liver, pancreas, heart, kidney, eye, and/or skin), an anti-TGFβ2/3 antibody of the invention can have one or more of the following properties:
an anti-TGFβ2/3 antibody comprising a heavy chain variable domain (VH) sequence having at least 90%, 91%, 92%, 93%, 94%, 95%, 96%, 97%, 98%, 99%, or 100% sequence identity to the amino acid sequence selected from the group consisting of SEQ ID NOs: 27, 81, 83, 86-88, 93-100, and 102-105, and a light chain variable domain (VL) having at least 90%, 91%, 92%, 93%, 94%, 95%, 96%, 97%, 98%, 99%, or 100% sequence identity to the amino acid sequence selected from the group consisting of SEQ ID NOs: 26, 80, 82, 84, 85, 89-92, and 101;
an anti-TGFβ2/3 antibody comprising the VH and VL sequences (respectively) selected from the group consisting of SEQ ID NOs: 27/26 (rabbit 4A11), SEQ ID NOs: 81/80 (v1), SEQ ID NOs: 81/82 (v2), SEQ ID NOs: 83/80 (v3), SEQ ID NOs: 83/82 (v4), SEQ ID NOs: 81/84 (v5), SEQ ID NOs: 81/85 (v6), SEQ ID NOs: 83/84 (v7), SEQ ID NOs: 86/84 (v7/1), SEQ ID NOs: 87/84 (v7.2), SEQ ID NOs: 88/84 (v7.3), SEQ ID NOs: 83/89 (v7.4), SEQ ID NOs: 83/90 (v7.5), SEQ ID NOs: 83/91 (v7.6), SEQ ID NOs: 83/92 (v7.7), SEQ ID NOs: 93/84 (v7.8), SEQ ID NOs: 94/84 (v7.9), SEQ ID NOs: 95/84 (v7.10), SEQ ID NOs: 96/84 (v7.11), SEQ ID NOs: 97/84 (v7.12), SEQ ID NOs: 98/84 (v7.13), SEQ ID NOs: 99/84 (v7.14), SEQ ID NOs: 100/84 (v7.15), SEQ ID NOs: 102/101 (v7.16), SEQ ID NOs: 103/101 (v7.17), SEQ ID NOs: 104/101 (v7.18), SEQ ID NOs: 105/101 (v7.19), SEQ ID NOs: 83/85 (v8);
an anti-TGFβ2/3 antibody comprising a complete H chain amino acid sequence having at least 95% sequence identity to the amino acid sequence selected from the group consisting of SEQ ID NOs: 33, 107, 109, 112-114, and 119-130 and/or a complete L chain amino acid sequence having an amino acid sequence selected from the group consisting of SEQ ID NOs: 32, 106, 108, 110, 111, 115-118, and 186;
an anti-TGFβ2/3 antibody comprising a complete H/L chain pair, the complete H/L chain pair (respectively) comprising amino acid sequences selected from the group consisting of SEQ ID NOs: 32/33 (rabbit 4A11), SEQ ID NOs: 107/106 (v1), SEQ ID NOs: 107/108 (v2), SEQ ID NOs: 109/106 (v3), SEQ ID NOs: 109/108 (v4), SEQ ID NOs: 107/110 (v5), SEQ ID NOs: 107/111 (v6), SEQ ID NOs: 109/110 (v7), SEQ ID NOs: 112/110 (v7.1), SEQ ID NOs: 113/110 (v7.2), SEQ ID NOs: 114/110 (v7.3), SEQ ID NOs: 114/115 (v7.4), SEQ ID NOs: 114/116 (v7.5), SEQ ID NOs: 114/117 (v7.6), SEQ ID NOs: 114/118 (v7.7), SEQ ID NOs: 119/110 (v7.8), SEQ ID NOs:120/110 (v7.9), SEQ ID NOs: 121/110 (v7.10), SEQ ID NOs: 122/110 (v7.11), SEQ ID NOs: 123/110 (v7.12), SEQ ID NOs: 124/110 (v7.13), SEQ ID NOs: 125/110 (v7.14), SEQ ID NOs: 126/110 (v7.15), SEQ ID NOs: 127/186 (v7.16), SEQ ID NOs: 128/186 (v7.17), SEQ ID NOs: 129/186 (v7.18), SEQ ID NOs: 130/186 (v7.19), and SEQ ID NOs: 114/111 (v8);
an antibody that binds to the same epitope as an anti-TGFβ2/3 antibody comprising (a) HVR-H1 comprising the amino acid sequence of SEQ ID NO: 10; (b) HVR-H2 comprising the amino acid sequence of SEQ ID NO: 11; (c) HVR-H3 comprising the amino acid sequence of SEQ ID NO: 12; (d) HVR-L1 comprising the amino acid sequence of SEQ ID NO: 13; (e) HVR-L2 comprising the amino acid sequence of SEQ ID NO: 14; and (f) HVR-L3 comprising an amino acid sequence selected from SEQ ID NO: 15;
an anti-TGFβ2/3 antibody that binds to an epitope within a fragment of TGFβ2 homodimer, the TGFβ2 homodimer having a first and a second TGFβ2 monomer, wherein the anti-TGFβ2/3 antibody comprises an antigen binding domain that directly contacts amino acid residues V313, Q314, D315, R320, L322, Y323, R328, D329, F345, and A347 of the first TGFβ2 monomer, and amino acid residues N368, T369, I370, N371, P372, E373, A374, S375, A376, and S377 of the second TGFβ2 monomer, and wherein, in some embodiments, the anti-TGFβ2/3 antibody binds to the same region and/or epitope in TGFβ3.

### Anti-TGFβ3 Antibodies

In any of the above aspects, with respect to the isoform-selective anti-TGFβ antibodies as described herein (e.g., anti-TGFβ2, anti-TGFβ2/3, or anti-TGFβ3 antibody) as a medicament (and manufacture or preparation or use thereof) and/or for use in a method of treatment, and/or for inhibiting TGFBR-dependent SMAD signaling, for inhibiting the assembly of TGFβ-TGFBR signaling complexes, for inhibiting TGFβ signaling through the TGFBR1/R2 complex, for inhibiting TGFβ signaling through the TGFBR2/ALK1 complex facilitated by endoglin in a subject, and/or for inhibiting new collagen synthesis e.g., for treating fibrosis (e.g., fibrosis of the lung, liver, pancreas, heart, kidney, eye, and/or skin), an anti-TGFβ3 antibody that selectively neutralizes TGFβ3 as described herein can have one or more of the following properties:
(a) the anti-TGFβ3 antibody binds to the beta6/beta7 hairpin region of TGFβ3;
(b) binding of the anti-TGFβ3 antibody sterically blocks the ability of TGFBR2, but not TGFBR1, to bind TGFβ3;
(c) binding of the anti-TGFβ3 antibody to TGFβ3 blocks TGFBR2 binding and inhibits the TGFBR1/TGFBR2 signaling receptors from binding to TGFβ3;
(d) the anti-TGFβ3 antibody binds amino acid residue R394 in the beta6/beta7 hairpin region of human TGFβ3;
(e) the anti-TGFβ3 antibody binds amino acid residue R394 in the beta6/beta7 hairpin region of human TGFβ3 and residue R394 of TGFβ3 makes an ionic salt bridge with the anti-TGFβ3 antibody in the heavy chain CDR2;
(f) isoform selectivity of the anti-TGFβ3 antibody for TGFβ3 over TGFβ1 is achieved by direct contact by the antigen binding domain of the anti-TGFβ3 antibody with amino acid residues T387, L389, and T395 of TGFβ3 (human TGFβ3 numbering);
(g) the isoform selectivity of the anti-TGFβ3 antibody for TGFβ3 over TGFβ2 is achieved by direct contact by the antigen binding domain of the anti-TGFβ3 antibody with amino acid residues R325, R394, and V398 of TGFβ3 (human TGFβ3 numbering);
(h) the anti-TGFβ3 antibody has reduced toxicity relative to the pan-TGFβ antibody 1D11;
(i) the anti-TGFβ3 antibody has reduced toxicity in rodents or cynomolgus monkeys relative to the pan-TGFβ antibody 1D11;
(j) the anti-TGFβ3 antibody has reduced toxicity relative to the pan-TGFβ small molecule inhibitor galunisertib;
(k) the anti-TGFβ3 antibody has reduced toxicity in rodents relative to the pan-TGFβ small molecule inhibitor galunisertib;
(l) the anti-TGFβ3 antibody has reduced toxicity relative to the anti-TGFβ1 antibody CAT-192;
(m) the anti-TGFβ3 antibody has reduced toxicity relative to an isoform selective anti-TGFβ2 antibody and/or anti-TGFβ2/3 antibody;
(n) the anti-TGFβ3 antibody comprises: (i) heavy chain CDRs comprising CDR-H1, -H2 and -H3, wherein CDR-H1 has the amino acid sequence of SEQ ID NO: 4, CDR-H2 has the amino acid sequence of one of SEQ ID NOs: 5, 34, 35, and 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9;
(o) the anti-TGFβ3 antibody comprises an antigen binding domain that directly contacts amino acid residues R325, K331, W332, H334, E335, T387, I388, L389, Y391, V392, G393, R394, P396, K397, and V398 on human TGFβ3; and
(p) the anti-TGFβ3 antibody as in (o), wherein the antigen binding domain is within 15-8, 8, 8-5, 7-5, 6-5, or 5 angstroms of the TGFβ3 amino acid residues; and
wherein in some embodiments, the use, medicament, or method is for treating fibrosis and wherein the fibrosis is SSc or IPF.

In further aspects, with respect to the isoform-selective anti-TGFβ antibodies as described herein (e.g., anti-TGFβ2, anti-TGFβ2/3, or anti-TGFβ3 antibody) as a medicament (and manufacture or preparation or use thereof) and/or for use in a method of treatment, and/or for inhibiting TGFBR-dependent SMAD signaling, for inhibiting the assembly of TGFβ-TGFBR signaling complexes, for inhibiting TGFβ signaling through the TGFBR1/R2 complex, for inhibiting TGFβ signaling through the TGFBR2/ALK1 complex facilitated by endoglin in a subject, and/or for inhibiting new collagen synthesis e.g., for treating fibrosis (e.g., fibrosis of the lung, liver, pancreas, heart, kidney, eye, and/or skin), an isoform-selective anti-TGFβ antibody of the invention can have one or more of the following properties:
an anti-TGFβ3 antibody that selectively neutralizes TGFβ3;
an anti-TGFβ3 antibody that binds to TGFβ3 with a K_{D} of less than 10 pM and/or has a cell-based IC₅₀ less than 250 pM;
an anti-TGFβ3 antibody that binds to TGFβ3 with a K_{D} of less than about 5 pM, about 4 pM, about 3 pM, or less than about 2 pM;
an anti-TGFβ3 antibody that has a cell-based IC₅₀ less of than about 200 pM, about 150 pM, about 100 pM, about 75 pM, or about 50 pM;
an anti-TGFβ3 antibody provided herein that has a cell-based IC₅₀ for inhibition (neutralization) of TGFβ3 of less than about 20 pM;
an anti-TGFβ3 antibody that selectively neutralizes TGFβ3 and has reduced toxicity in mice relative to the pan-TGFβ antibody 1D11; and
an anti-TGFβ3 antibody that selectively neutralizes TGFβ3 and has an improved safety profile; and
wherein in some embodiments, the use, medicament, or method is for treating fibrosis and wherein the fibrosis is SSc or IPF.

In any of the above aspects, with respect to the isoform-selective anti-TGFβ antibodies as described herein (e.g., anti-TGFβ2, anti-TGFβ2/3, or anti-TGFβ3 antibody) as a medicament (and manufacture or preparation or use thereof) and/or for use in a method of treatment, and/or for inhibiting TGFBR-dependent SMAD signaling, for inhibiting the assembly of TGFβ-TGFBR signaling complexes, for inhibiting TGFβ signaling through the TGFBR1/R2 complex, for inhibiting TGFβ signaling through the TGFBR2/ALK1 complex facilitated by endoglin in a subject, and/or for inhibiting new collagen synthesis e.g., for treating fibrosis (e.g., fibrosis of the lung, liver, pancreas, heart, kidney, eye, and/or skin), an anti-TGFβ3 antibody of the invention can have one or more of the following properties:
an anti-TGFβ3 antibody comprising at least one, two, three, four, five, or six HVRs selected from (a) HVR-H1 comprising the amino acid sequence of SEQ ID NO: 4; (b) HVR-H2 comprising the amino acid sequence selected from SEQ ID NOs: 5, 34, 35, and 159; (c) HVR-H3 comprising the amino acid sequence of SEQ ID NO: 6; (d) HVR-L1 comprising the amino acid sequence of SEQ ID NO: 7; (e) HVR-L2 comprising the amino acid sequence of SEQ ID NO: 8; and (f) HVR-L3 comprising the amino acid sequence of SEQ ID NO: 9;
an anti-TGFβ3 antibody comprising (a) HVR-H1 comprising the amino acid sequence of SEQ ID NO: 4; (b) HVR-H2 comprising the amino acid sequence of SEQ ID NO: 5; (c) HVR-H3 comprising the amino acid sequence of SEQ ID NO: 6; (d) HVR-L1 comprising the amino acid sequence of SEQ ID NO: 7; (e) HVR-L2 comprising the amino acid sequence of SEQ ID NO: 8; and (f) HVR-L3 comprising an amino acid sequence selected from SEQ ID NO: 9;
an anti-TGFβ3 antibody comprising (a) HVR-H1 comprising the amino acid sequence of SEQ ID NO: 4; (b) HVR-H2 comprising the amino acid sequence of SEQ ID NO: 34; (c) HVR-H3 comprising the amino acid sequence of SEQ ID NO: 6; (d) HVR-L1 comprising the amino acid sequence of SEQ ID NO: 7; (e) HVR-L2 comprising the amino acid sequence of SEQ ID NO: 8; and (f) HVR-L3 comprising an amino acid sequence selected from SEQ ID NO: 9;
an anti-TGFβ3 antibody comprising (a) HVR-H1 comprising the amino acid sequence of SEQ ID NO: 4; (b) HVR-H2 comprising the amino acid sequence of SEQ ID NO: 35; (c) HVR-H3 comprising the amino acid sequence of SEQ ID NO: 6; (d) HVR-L1 comprising the amino acid sequence of SEQ ID NO: 7; (e) HVR-L2 comprising the amino acid sequence of SEQ ID NO: 8; and (f) HVR-L3 comprising an amino acid sequence selected from SEQ ID NO: 9;
an anti-TGFβ3 antibody comprising (a) HVR-H1 comprising the amino acid sequence of SEQ ID NO: 4; (b) HVR-H2 comprising the amino acid sequence of SEQ ID NO: 159; (c) HVR-H3 comprising the amino acid sequence of SEQ ID NO: 6; (d) HVR-L1 comprising the amino acid sequence of SEQ ID NO: 7; (e) HVR-L2 comprising the amino acid sequence of SEQ ID NO: 8; and (f) HVR-L3 comprising an amino acid sequence selected from SEQ ID NO: 9.

In certain embodiments, any one or more amino acids of an anti-TGFβ3 antibody as provided above are substituted at the following HVR positions:
in HVR-H2 (SEQ ID NO: 5): at position N54 (e.g., N54S, N54Q) or T56 (e.g., T56A). In certain embodiments, the substitutions are conservative substitutions, as provided herein.

In any of the above embodiments, an anti-TGFβ3 antibody may be humanized. In one embodiment, an anti-TGFβ3 antibody comprises HVRs as in any of the above embodiments, and further comprises an acceptor human framework, e.g. a human immunoglobulin framework or a human consensus framework. In another embodiment, an anti-TGFβ3 antibody comprises HVRs as in any of the above embodiments, and further comprises a VH comprising FR modifications selected from the group consisting of 47L or 47W; 49A, 49S, or 49G; 73D or 73N; and 76N, 78D or 78L, 78A, or 78V. In some embodiments, the anti-TGFβ3 antibody comprises a VH, wherein the VH comprises the amino acid sequence of SEQ ID NO: 23, wherein the VH comprises a set of framework modifications selected from the group consisting of: (i) 47L, 49A in FR2, 78V in FR3 (h2A10.v1); (ii) 47L, 49A in FR2, 73D, 76S, 78V in FR3 (h2A10.v2); (iii) 47W in FR2 (h2A10.v1.5); (iv): 49G in FR2 (h2A10.v1.6); (v) 78A in FR3 (h2A10.v1.7); (vi) 47W in FR2 (h2A10.v2.5); (vii) 49S in FR2 (h2A10.v2.6); (viii) 73N in FR3 (h2A10.v2.7); (ix) 76N in FR3 (h2A10.v2.8); (x) 78L in FR3 (h2A10.v2.9); and (xi) 49S in FR2, 76N, 78L in FR3 (h2A10.v3 and h2A10.v4). In another embodiment, an anti-TGFβ3 antibody comprises HVRs as in any of the above embodiments, and further comprises a VL comprising FR modifications selected from the group consisting of: 4L or 4M, 38H or 38Q, 43A or 43Q, and 58V, relative to the VL having the amino acid sequence of SEQ ID NO: 22. In another embodiment, the antibody comprises the VL of SEQ ID NO: 22, wherein the VL comprises a set of framework modifications selected from the group consisting of: (i) 4L in FR1, 38H and 43Q in FR2, 58I in FR3 (h2A10.v1 and h2A10.v2); (ii) 4M in FR1 (h2A10.v1.1 and h2A10.v2.1); (iii) 38Q in FR2 (h2A10.v1.2 and h2A10.v2.2); (iv) 43A in FR2 (h2A10.v1.3 and h2A10.v2.3); (v) 58V in FR3 (h2A10.v1.4 and h2A10.v2.4); (vi) 38Q, 43A in FR2, 58V in FR3 (h2A10.v3 and h2A10.v4); (vii) 58V in FR3 (h2A10.v1.4 and h2A10.v2.4); and (vi) 38Q, 43A in FR2, 58V in FR3 (h2A10.v3 and h2A10.v4).

In any of the above aspects, with respect to the isoform-selective anti-TGFβ antibodies as described herein (e.g., anti-TGFβ2, anti-TGFβ2/3, or anti-TGFβ3 antibody) as a medicament (and manufacture or preparation or use thereof) and/or for use in a method of treatment, and/or for inhibiting TGFBR-dependent SMAD signaling, for inhibiting the assembly of TGFβ-TGFBR signaling complexes, for inhibiting TGFβ signaling through the TGFBR1/R2 complex, for inhibiting TGFβ signaling through the TGFBR2/ALK1 complex facilitated by endoglin in a subject, and/or for inhibiting new collagen synthesis e.g., for treating fibrosis (e.g., fibrosis of the lung, liver, pancreas, heart, kidney, eye, and/or skin), an isoform-selective anti-TGFβ antibody of the invention can have one or more of the following properties:
an anti-TGFβ3 antibody comprising a heavy chain variable domain (VH) sequence having at least 90%, 91%, 92%, 93%, 94%, 95%, 96%, 97%, 98%, 99%, or 100% sequence identity to the amino acid sequence selected from the group consisting of SEQ ID NOs: 23, 37, and 42-50, and a light chain variable domain (VL) having at least 90%, 91%, 92%, 93%, 94%, 95%, 96%, 97%, 98%, 99%, or 100% sequence identity to the amino acid sequence selected from the group consisting of SEQ ID NOs: 22, 36, and 38-41;
an anti-TGFβ3 antibody comprising VH/VL sequences (respectively) selected from the group consisting of SEQ ID NOs: 23/22 (rat 2A10), SEQ ID NOs: 37/36 (v1), SEQ ID NOs: 37/38 (v1.1), SEQ ID NOs: 37/39 (v1.2), SEQ ID NOs: 37/40 (v1.3), SEQ ID NOs: 37/41 (v1.4), SEQ ID NOs: 42/36 (v1.5), SEQ ID NOs: 43/36 (v1.6), SEQ ID NOs:44/36 (v1.7), SEQ ID NOs: 45/36 (v2), SEQ ID NOs: 45/38 (v2.1), SEQ ID NOs: 45/39 (v2.2), SEQ ID NOs: 45/40 (v2.3), SEQ ID NOs: 45/41 (v2.4), SEQ ID NOs: 46/36 (v2.5), SEQ ID NOs: 47/36 (v2.6), SEQ ID NOs: 48/36 (v2.7), SEQ ID NOs: 49/36 (v2.8), and SEQ ID NOs: 50/36 (v2.9);
an anti-TGFβ3 antibody comprising a heavy chain variable domain (VH) sequence having at least 90%, 91%, 92%, 93%, 94%, 95%, 96%, 97%, 98%, 99%, or 100% sequence identity to the amino acid sequence selected from the group consisting of SEQ ID NOs: 23, 37, 42-50 55, and 57, and a light chain variable domain (VL) having at least 90%, 91%, 92%, 93%, 94%, 95%, 96%, 97%, 98%, 99%, or 100% sequence identity to the amino acid sequence selected from the group consisting of SEQ ID NOs: 22, 36, 38-41, 54, and 56;
an anti-TGFβ3 antibody comprising VH/VL sequences (respectively) selected from the group consisting of SEQ ID NOs: 23/22 (rat 2A10), SEQ ID NOs: 37/36 (v1), SEQ ID NOs: 37/38 (v1.1), SEQ ID NOs: 37/39 (v1.2), SEQ ID NOs: 37/40 (v1.3), SEQ ID NOs: 37/41 (v1.4), SEQ ID NOs: 42/36 (v1.5), SEQ ID NOs: 43/36 (v1.6), SEQ ID NOs:44/36 (v1.7), SEQ ID NOs: 45/36 (v2), SEQ ID NOs: 45/38 (v2.1), SEQ ID NOs: 45/39 (v2.2), SEQ ID NOs: 45/40 (v2.3), SEQ ID NOs: 45/41 (v2.4), SEQ ID NOs: 46/36 (v2.5), SEQ ID NOs: 47/36 (v2.6), SEQ ID NOs: 48/36 (v2.7), SEQ ID NOs: 49/36 (v2.8), SEQ ID NOs: 50/36 (v2.9), SEQ ID NOs: 55/54 (v3), and SEQ ID NOs: 57/56 (v4);
an anti-TGFβ3 antibody comprises a complete H chain amino acid sequence having at least 95% sequence identity to the amino acid sequence selected from the group consisting of SEQ ID NOs: 29, 59, 64, and 65-72 and/or a complete L chain amino acid sequence having an amino acid sequence selected from the group consisting of SEQ ID NOs: 28, 58, and 60-63;
an anti-TGFβ3 antibody comprising a complete H/L chain pair, the complete H/L chain pair (respectively) comprising amino acid sequences selected from the group consisting of SEQ ID NOs: 29/28 (rat 2A10), SEQ ID NOs: 59/58 (vi), SEQ ID NOs: 59/60 (v1.1), SEQ ID NOs: 59/61 (v1.2), SEQ ID NOs: 59/62 (v1.3), SEQ ID NOs: 59/63 (v1.4), SEQ ID NOs: 64/58 (v1.5), SEQ ID NOs: 65/58 (v1.6), SEQ ID NOs: 66/58 (v1.7), SEQ ID NOs: 67/58 (v2), SEQ ID NOs: 67/60 (v2.1), SEQ ID NOs: 67/61 (v2.2), SEQ ID NOs: 67/62 (v2.3), SEQ ID NOs: 67/63 (v2.4), SEQ ID NOs: 68/58 (v2.5), SEQ ID NOs: 69/58 (v2.6), SEQ ID NOs: 70/58 (v2.7), SEQ ID NOs: 71/58 (v2.8), SEQ ID NOs: 72/58 (v2.9);
an anti-TGFβ3 antibody comprising a complete H chain amino acid sequence having at least 95% sequence identity to the amino acid sequence selected from the group consisting of SEQ ID NOs: 29, 59, 64, 65-72, 77 and 79 and/or a complete L chain amino acid sequence having an amino acid sequence selected from the group consisting of SEQ ID NOs: 28, 58, 60-63, 76, and 78;
an anti-TGFβ3 antibody comprising a complete H/L chain pair, the complete H/L chain pair (respectively) comprising amino acid sequences selected from the group consisting of SEQ ID NOs: 29/28 (rat 2A10), SEQ ID NOs: 59/58 (v1), SEQ ID NOs: 59/60 (v1.1), SEQ ID NOs: 59/61 (v1.2), SEQ ID NOs: 59/62 (v1.3), SEQ ID NOs: 59/63 (v1.4), SEQ ID NOs: 64/58 (v1.5), SEQ ID NOs: 65/58 (v1.6), SEQ ID NOs: 66/58 (v1.7), SEQ ID NOs: 67/58 (v2), SEQ ID NOs: 67/60 (v2.1), SEQ ID NOs: 67/61 (v2.2), SEQ ID NOs: 67/62 (v2.3), SEQ ID NOs: 67/63 (v2.4), SEQ ID NOs: 68/58 (v2.5), SEQ ID NOs: 69/58 (v2.6), SEQ ID NOs: 70/58 (v2.7), SEQ ID NOs: 71/58 (v2.8), SEQ ID NOs: 72/58 (v2.9), SEQ ID NOs: 77/76 (v3), and SEQ ID NOs: 79/78;
an anti-TGFβ3 antibody comprising a VH having at least 90%, 91%, 92%, 93%, 94%, 95%, 96%, 97%, 98%, 99%, or 100% sequence identity to the amino acid sequence of SEQ ID NO: 52 and a VL amino acid sequence having at least 90%, 91%, 92%, 93%, 94%, 95%, 96%, 97%, 98%, 99%, or 100% sequence identity to an amino acid sequence selected from the group consisting of SEQ ID NOs: 22, 36, and 38-41;
an anti-TGFβ3 antibody comprising VH/VL sequences (respectively) comprising the amino acid sequences of SEQ ID NOs: 52/36 (h2A10.v2.N54Q);
an anti-TGFβ3 antibody comprising a complete H chain having at least 90%, 91%, 92%, 93%, 94%, 95%, 96%, 97%, 98%, 99%, or 100% sequence identity to the amino acid sequence of SEQ ID NO: 74 and a complete L chain amino acid sequence having at least 90%, 91%, 92%, 93%, 94%, 95%, 96%, 97%, 98%, 99%, or 100% sequence identity to an amino acid sequence selected from the group consisting of SEQ ID NOs: 28, 58, and 60-63;
an anti-TGFβ3 antibody comprising complete H/L chain sequences (respectively) comprising the amino acid sequences of SEQ ID NOs: 74/58 (h2A10.v2.N54Q);
an anti-TGFβ3 antibody comprising a VH having at least 90%, 91%, 92%, 93%, 94%, 95%, 96%, 97%, 98%, 99%, or 100% sequence identity to the amino acid sequence of SEQ ID NO: 51 or 55 and a VL amino acid sequence having at least 90%, 91%, 92%, 93%, 94%, 95%, 96%, 97%, 98%, 99%, or 100% sequence identity to an amino acid sequence selected from the group consisting of SEQ ID NOs: 22, 36, and 38-41;
an anti-TGFβ3 antibody comprising a VH having at least 90%, 91%, 92%, 93%, 94%, 95%, 96%, 97%, 98%, 99%, or 100% sequence identity to the amino acid sequence of SEQ ID NO: 51 or 55 and a VL amino acid sequence having at least 90%, 91%, 92%, 93%, 94%, 95%, 96%, 97%, 98%, 99%, or 100% sequence identity to an amino acid sequence selected from the group consisting of SEQ ID NOs: 22, 36, 38-41, and 54;
an anti-TGFβ3 antibody comprising VH/VL sequences (respectively) comprising the amino acid sequences of SEQ ID NO: 51/36 (h2A10.v2.N54S) or SEQ ID NO: 55/54 (h2A10.v3);
an anti-TGFβ3 antibody comprising a complete H chain having at least 95% sequence identity to the amino acid sequence of SEQ ID NO: 73 or 77 and/or the anti-TGFβ3 antibody comprises a complete L chain amino acid sequence having at least 90%, 91%, 92%, 93%, 94%, 95%, 96%, 97%, 98%, 99%, or 100% sequence identity to an amino acid sequence selected from the group consisting of SEQ ID NOs: 28, 58, and 60-63;
an anti-TGFβ3 antibody comprising a complete H chain having at least 95% sequence identity to the amino acid sequence of SEQ ID NO: 73 or 77 and/or the anti-TGFβ3 antibody comprises a complete L chain amino acid sequence having at least 90%, 91%, 92%, 93%, 94%, 95%, 96%, 97%, 98%, 99%, or 100% sequence identity to an amino acid sequence selected from the group consisting of SEQ ID NOs: 28, 58, 60-63, and 76;
an anti-TGFβ3 antibody comprising complete H/L chain sequences (respectively) comprising the amino acid sequences of SEQ ID NOs: 73/58 (h2A10.v2.N54S) or SEQ ID NOs: 77/76 (h2A10.v3);
an anti-TGFβ3 antibody comprising a VH having at least 90%, 91%, 92%, 93%, 94%, 95%, 96%, 97%, 98%, 99%, or 100% sequence identity to the amino acid sequence of SEQ ID NO: 53 or 57, and a VL amino acid sequence having at least 90%, 91%, 92%, 93%, 94%, 95%, 96%, 97%, 98%, 99%, or 100% sequence identity to an amino acid sequence selected from the group consisting of SEQ ID NOs: 22, 36, and 38-41;
an anti-TGFβ3 antibody comprising a VH having at least 90%, 91%, 92%, 93%, 94%, 95%, 96%, 97%, 98%, 99%, or 100% sequence identity to the amino acid sequence of SEQ ID NO: 53 or 57, and a VL amino acid sequence having at least 90%, 91%, 92%, 93%, 94%, 95%, 96%, 97%, 98%, 99%, or 100% sequence identity to an amino acid sequence selected from the group consisting of SEQ ID NOs: 22, 36, 38-41, and 56;
an anti-TGFβ3 antibody comprising VH/VL sequences (respectively) comprising the amino acid sequences of SEQ ID NOs: 53/36 (h2A10.v2.T56A) or SEQ ID NOs: 57/56 (h2A10.v4);
an anti-TGFβ3 antibody comprising a complete H chain having at least 95% sequence identity to the amino acid sequence of SEQ ID NO: 74 or 79 and/or a complete L chain amino acid sequence having at least 90%, 91%, 92%, 93%, 94%, 95%, 96%, 97%, 98%, 99%, or 100% sequence identity to an amino acid sequence selected from the group consisting of SEQ ID NOs: 28, 58, and 60-63;
an anti-TGFβ3 antibody comprising a complete H chain having at least 95% sequence identity to the amino acid sequence of SEQ ID NO: 74 or 79 and/or a complete L chain amino acid sequence having at least 90%, 91%, 92%, 93%, 94%, 95%, 96%, 97%, 98%, 99%, or 100% sequence identity to an amino acid sequence selected from the group consisting of SEQ ID NOs: 28, 58, 60-63, and 78;
an anti-TGFβ3 antibody comprising complete H/L chain sequences (respectively) comprising the amino acid sequences of SEQ ID NOs: 75/58 (h2A10.v2.T56A) or SEQ ID NOs: 79/78 (h2A10.v4);
an anti-TGFβ3 antibody that binds to the same epitope as an anti-TGFβ3 antibody comprising (a) HVR-H1 comprising the amino acid sequence of SEQ ID NO: 4; (b) HVR-H2 comprising the amino acid sequence of SEQ ID NO: 5; and (c) HVR-H3 comprising the amino acid sequence of SEQ ID NO:6;
an anti-TGFβ3 antibody that binds to the same epitope as an anti-TGFβ3 antibody comprising (a) HVR-H1 comprising the amino acid sequence of SEQ ID NO: 4; (b) HVR-H2 comprising the amino acid sequence of SEQ ID NO: 34; and (c) HVR-H3 comprising the amino acid sequence of SEQ ID NO:6;
an anti-TGFβ3 antibody that binds to the same epitope as an anti-TGFβ3 antibody comprising (a) HVR-H1 comprising the amino acid sequence of SEQ ID NO: 4; (b) HVR-H2 comprising the amino acid sequence of SEQ ID NO: 35; and (c) HVR-H3 comprising the amino acid sequence of SEQ ID NO:6;
an anti-TGFβ3 antibody that binds to the same epitope as an anti-TGFβ3 antibody comprising (a) HVR-H1 comprising the amino acid sequence of SEQ ID NO: 4; (b) HVR-H2 comprising the amino acid sequence of SEQ ID NO: 159; and (c) HVR-H3 comprising the amino acid sequence of SEQ ID NO:6;
an anti-TGFβ3 antibody that binds to the same epitope as an anti-TGFβ3 antibody comprising VH/VL sequences (respectively) selected from the group consisting of SEQ ID NOs: 53/36 (h2A10.v2.t56A), SEQ ID NOs: 57/56 (h2A10.v4), SEQ ID NOs: 51/36 (h2A10.v2.N54S), SEQ ID NOs: 55/54 (h2A10.v3), SEQ ID NOs: 52/36 (h2A10.v2.N54Q), SEQ ID NOs: 23/22 (rat 2A10), SEQ ID NOs: 37/36 (v1), SEQ ID NOs: 37/38 (v1.1), SEQ ID NOs: 37/39 (v1.2), SEQ ID NOs: 37/40 (v1.3), SEQ ID NOs: 37/41 (v1.4), SEQ ID NOs: 42/36 (v1.5), SEQ ID NOs: 43/36 (v1.6), SEQ ID NOs:44/36 (v1.7), SEQ ID NOs: 45/36 (v2), SEQ ID NOs: 45/38 (v2.1), SEQ ID NOs: 45/39 (v2.2), SEQ ID NOs: 45/40 (v2.3), SEQ ID NOs: 45/41 (v2.4), SEQ ID NOs: 46/36 (v2.5), SEQ ID NOs: 47/36 (v2.6), SEQ ID NOs: 48/36 (v2.7), SEQ ID NOs: 49/36 (v2.8), and SEQ ID NOs: 50/36 (v2.9);
an anti-TGFβ3 antibody that binds to the same epitope as an anti-TGFβ3 antibody comprising VH/VL sequences (respectively) selected from the group consisting of SEQ ID NOs: 53/36 (h2A10.v2.t56A), SEQ ID NOs: 57/56 (h2A10.v4), SEQ ID NOs: 51/36 (h2A10.v2.N54S), SEQ ID NOs: 55/54 (h2A10.v3), SEQ ID NOs: 52/36 (h2A10.v2.N54Q), SEQ ID NOs: 23/22 (rat 2A10), SEQ ID NOs: 37/36 (v1), SEQ ID NOs: 37/38 (v1.1), SEQ ID NOs: 37/39 (v1.2), SEQ ID NOs: 37/40 (v1.3), SEQ ID NOs: 37/41 (v1.4), SEQ ID NOs: 42/36 (v1.5), SEQ ID NOs: 43/36 (v1.6), SEQ ID NOs:44/36 (v1.7), SEQ ID NOs: 45/36 (v2), SEQ ID NOs: 45/38 (v2.1), SEQ ID NOs: 45/39 (v2.2), SEQ ID NOs: 45/40 (v2.3), SEQ ID NOs: 45/41 (v2.4), SEQ ID NOs: 46/36 (v2.5), SEQ ID NOs: 47/36 (v2.6), SEQ ID NOs: 48/36 (v2.7), SEQ ID NOs: 49/36 (v2.8), and SEQ ID NOs: 50/36 (v2.9), SEQ ID NOs: 55/54 (v3), and SEQ ID NOs: 57/56 (v4);
an anti-TGFβ3 antibody that binds to the same epitope as an anti-TGFβ3 antibody comprising VH/VL sequences (respectively) of SEQ ID NOs: 57/56 (h2A10.v4);
an anti-TGFβ3 antibody that comprises an antigen binding domain that directly contacts amino acid residues R325, K331, W332, H334, E335, T387, I388, L389, Y391, V392, G393, R394, P396, K397, and V398 on human TGFβ3, wherein the anti-TGFβ3 antibody selectively neutralizes TGFβ3;
an anti-TGFβ3 antibody that selectively neutralizes TGFβ3 and has reduced toxicity in mice relative to the pan-TGFβ antibody 1D11;
an anti-TGFβ3 antibody that has reduced toxicity in mice relative to the pan-TGFβ antibody 1D11 at a dose of 50 mg/kg.

An isoform-selective anti-TGFβ antibody according to any of the above uses (e.g. as a medicament for treating fibrosis) may be a monoclonal antibody, including a chimeric, humanized or human antibody. In one embodiment, an isoform-selective anti-TGFβ antibody is an antibody fragment, e.g., a Fv, Fab, Fab′, scFv, diabody, or F(ab′)₂ fragment. In another embodiment, the antibody is a full length antibody, e.g., an intact IgG1 or IgG4, preferably an IgG1, antibody or other antibody class or isotype as defined herein.

In any of the aspects provided herein, for use of an anti-TGFβ3 antibody as a medicament (and the manufacture or preparation or use thereof) and/or for use in a method of treatment, and/or for inhibiting TGFBR-dependent SMAD signaling, for inhibiting the assembly of TGFβ-TGFBR signaling complexes, for inhibiting TGFβ signaling through the TGFBR1/R2 complex, for inhibiting TGFβ signaling through the TGFBR2/ALK1 complex facilitated by endoglin in a subject, and/or for inhibiting new collagen synthesis e.g., for treating fibrosis (e.g., fibrosis of the lung, liver, pancreas, heart, kidney, eye, and/or skin), the anti-TGFβ3 antibody is one that selectively neutralizes TGFβ3 as described herein and comprises: heavy chain CDRs comprising CDR-H1, -H2 and -H3, wherein CDR-H1 has the amino acid sequence of SEQ ID NO: 4, CDR-H2 has the amino acid sequence of one of SEQ ID NOs: 5, 34, 35, and 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9. In some embodiments, the CDR-H2 has the amino acid sequence of SEQ ID NO: 35. In certain embodiments, the anti-TGFβ3 antibody further comprises a VH having at least 90%, 91%, 92%, 93%, 94%, 95%, 96%, 97%, 98%, 99%, or 100% sequence identity to the amino acid sequence of SEQ ID NO: 57. In some embodiments, the anti-TGFβ3 antibody further comprises a VL having at least 90%, 91%, 92%, 93%, 94%, 95%, 96%, 97%, 98%, 99%, or 100% sequence identity to the amino acid sequence of SEQ ID NO: 56. In certain embodiments, the anti-TGFβ3 antibody further comprises a complete H chain having at least 90%, 91%, 92%, 93%, 94%, 95%, 96%, 97%, 98%, 99%, or 100% sequence identity to an amino acid sequence of SEQ ID NO: 79. In other embodiments, the anti-TGFβ3 antibody further comprises a complete L chain having at least 90%, 91%, 92%, 93%, 94%, 95%, 96%, 97%, 98%, 99%, or 100% sequence identity to an amino acid sequence of SEQ ID NO: 78. In some embodiments, the disorder is fibrosis. In certain embodiments, the disorder is SSc. In some embodiments, the disorder is IPF. In certain embodiments, the anti-TGFβ3 antibody is a monoclonal antibody, such as a chimeric, humanized or human antibody. In some embodiments, the anti-TGFβ3 antibody is an antibody fragment, e.g., a Fv, Fab, Fab′, scFv, diabody, or F(ab′)₂ fragment. In other embodiments, the anti-TGFβ3 antibody is a full length antibody, e.g., an intact IgG1 or IgG4 antibody, such as an IgG1, or other antibody class or isotype as defined herein.

### Uses of Pharmaceutical Formulations

In a further aspect, the invention provides pharmaceutical formulations comprising any of the isoform-selective anti-TGFβ antibodies (e.g., anti-TGFβ2, anti-TGFβ2/3, or anti-TGFβ3 antibodies) provided herein, e.g., for use in any of the above therapeutic methods, uses and medicaments. In one embodiment, a pharmaceutical formulation comprises any of the isoform-selective anti-TGFβ antibodies (e.g., anti-TGFβ2, anti-TGFβ2/3, or anti-TGFβ3 antibodies) provided herein and a pharmaceutically acceptable carrier. In another embodiment, a pharmaceutical formulation comprises any of the isoform-selective anti-TGFβ antibodies (e.g., anti-TGFβ2, anti-TGFβ2/3, or anti-TGFβ3 antibodies, e.g., an antibody having VL and VH amino acid sequence as shown in any of FIGS. 12-16 and 18-24 or Tables 5, 6, 7, 12, 13, 14, 19, 20, 23 and 24) provided herein and at least one additional therapeutic agent, e.g., as described below.

### Combination Therapies

Antibodies of the invention can be used either alone or in combination with other agents in a therapy. For instance, an antibody of the invention may be co-administered with at least one additional therapeutic agent. In some embodiments, the therapeutic agent is selected from pirfenidone, nintedanib, mycophenylate mofetil, an IL-6 inhibitor (e.g., tocilizumab, sarilumab), an anti-CTFG antibody (e.g., FG-3019), an autotaxin inhibitor, a JAK inhibitor, an IL-11 inhibitor, and serum amyloid P (PTX2). See, Raghu et al. European Respiratory Journal 2012 40: 2819) (FG-309, Ninou et al. (2018) Front Med (Lausanne); 5: 180 (autotaxin inhibitors). In a preferred embodiment, an anti-TGFβ antibody (e.g., anti-TGFβ2, anti-TGFβ2/3, or anti-TGFβ3 antibody) is administered to a subject in need thereof in combination with tocilizumab. In a preferred embodiment, the subject in need thereof is a patient suffering from SSc. In some embodiments, the anti-TGFβ antibody is an anti-TGFβ3 antibody that selectively neutralizes TGFβ3. Therapeutic dosages of tocilizumab which may be administered to a subject, e.g., in combination with an anti-TGFβ3 antibody (or anti-TGFβ2 or anti-TGFβ2/3 antibody) according to the present invention are described in U.S. Pat. No. 9,539,263, and includes, e.g., a dose of about 162 mg per dose, administered, e.g., every week or ever two weeks, wherein the preferred route of administration is subcutaneous.

In certain embodiments, a subject is administered an anti-TGFβ antibody of the invention in combination with a therapeutic agent for the treatment of IPF. Certain therapeutic agents have been previously described as candidates or agents for the treatment of IPF. These have been described in the published literature and are reviewed, for example, in Rafli et al., J. Thorac. Dis (2013) 5(1):48-73. Such agents include agents that have antioxidant, immunosuppressant and/or anti-inflammatory activities such as N-acetylcysteine; agents that have antifibrotic, anti-inflammatory and/or antioxidant activities such as pirfenidone, an orally administered pyridine which has been approved for clinical use in the treatment of IPF; or an antibody against αvβ6 integrin (e.g., STX-100); agents that inhibit connective tissue growth factor (CTGF), such as an anti-CTGF antibody (e.g., FG-3019); agents that inhibit somatostatin receptors, such as somatostatin analogs (e.g, SOM230, octreotide); agents that inhibit IL-13, IL-4 and CCL2, such as an anti-IL13 antibody (e.g., QAX576, tralokinumab, lebrikizumab), an anti-IL4 antibody, a combination anti-IL13/anti-IL4 agent (e.g., a bispecific anti-IL13/anti-IL4 antibody such as SAR156597), an anti-IL-6 inhibitor (e.g., tocilizumab, sarilumab), an anti-CCL2 antibody (e.g., CNT0888); agents that have anti-angiogenic, immunomodulatory, and/or anti-inflammatory activities such as thalidomide or minocycline; agents that inhibit the enzyme lysyl oxidase-like 2 (LOXL2), such as an anti-LOXL2 antibody (e.g., GS-6624 [simtuzumab]); agents that inhibit angiogenesis such as the tyrosine kinase inhibitor, BIBF 1120, tetrathiomolybdate; agents that inhibit deposition of extracellular matrix and/or disrupt collagen deposition, such as doxycycline; agents that target the renin-angiotensin system such as losartan; and other agents having anti-proliferative and/or anti-fibrotic activities such as carbon monoxide.

Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent or agents. In one embodiment, administration of the isoform-selective anti-TGFβ antibody (e.g., anti-TGFβ2, anti-TGFβ2/3, or anti-TGFβ3 antibody) and administration of an additional therapeutic agent (e.g., an anti-IL-6 inhibitor (e.g., tozilumab, sarilumab) occur within about one month, or within about one, two or three weeks, or within about one, two, three, four, five, or six days, of each other.

In a specific embodiment, a combination therapy comprises administering to a subject in need of such combination therapy, e.g., a patient diagnosed as having a TGFβ-related disorder, e.g., fibrosis, e.g., IPF, COPD, PF-ILD (e.g., SSc), hepatic fibrosis (e.g., liver cirrhosis or chronic hepatic fibrosis, or other fibrotic condition or cancer described herein), a therapeutically effective amount of an anti-TGFβ antibody of the present invention (e.g., an anti-TGFβ2, an anti-TGFβ2/3, or an anti-TGFβ3 antibody) in combination with an agent selected from the group consisting of: N-acetylcysteine; agents that have antifibrotic, anti-inflammatory and/or antioxidant activities such as pirfenidone, an orally administered pyridine which has been approved for clinical use in the treatment of IPF; or an antibody against αvβ6 integrin (e.g., STX-100); agents that inhibit connective tissue growth factor (CTGF), such as an anti-CTGF antibody (e.g., FG-3019); agents that inhibit somatostatin receptors, such as somatostatin analogs (e.g, SOM230, octreotide); agents that inhibit IL-13, IL-4 and CCL2, such as an anti-IL13 antibody (e.g., QAX576, tralokinumab, lebrikizumab), an anti-IL4 antibody, a combination anti-IL13/anti-IL4 agent (e.g., a bispecific anti-IL13/anti-IL4 antibody such as SAR156597), an anti-IL-6 inhibitor (e.g., tozilumab, sarilumab), an anti-CCL2 antibody (e.g., CNT0888); agents that have anti-angiogenic, immunomodulatory, and/or anti-inflammatory activities such as thalidomide or minocycline; agents that inhibit the enzyme lysyl oxidase-like 2 (LOXL2), such as an anti-LOXL2 antibody (e.g., GS-6624 [simtuzumab]); agents that inhibit angiogenesis such as the tyrosine kinase inhibitor, BIBF 1120, tetrathiomolybdate; agents that inhibit deposition of extracellular matrix and/or disrupt collagen deposition, such as doxycycline; agents that target the renin-angiotensin system such as losartan; and other agents having anti-proliferative and/or anti-fibrotic activities such as carbon monoxide.

### Administration and Formulation

An antibody of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. In preferred embodiments, the administration of an anti-TGFβ antibody described herein is subcutaneous or intravenous. Various dosing schedules including but not limited to single or multiple administrations over various time-points, bolus administration, and pulse infusion are contemplated herein.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the use of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g., 0.1 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. However, other dosage regimens may be useful. The progress of this therapy is easily monitored by conventional techniques and assays.

A “subject” or “individual” according to any of the above embodiments may be a human. It is understood that any of the above formulations or therapeutic methods may be carried out using an immunoconjugate of the invention in place of or in addition to an isoform-selective anti-TGFβ antibody (e.g., anti-TGFβ2, anti-TGFβ2/3, or anti-TGFβ3 antibody).

### H. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate of the invention in place of or in addition to an isoform-selective anti-TGFβ antibody (e.g., anti-TGFβ2, anti-TGFβ2/3, or anti-TGFβ3 antibody).

### III. Enumerated Embodiments

Embodiment 1. An isolated anti-tumor necrosis factor beta 3 (TGFβ3) antibody, wherein the antibody selectively neutralizes TGFβ3, and wherein the antibody comprises one or more of the following features:
(a) the anti-TGFβ3 antibody specifically binds to the beta6/beta7 hairpin region of TGFβ3;
(b) binding of the anti-TGFβ3 antibody sterically blocks the ability of TGFBR2, but not TGFBR1, to bind TGFβ3;
(c) binding of the anti-TGFβ3 antibody to TGFβ3 blocks TGFBR2 binding and inhibits the TGFBR1/TGFBR2 signaling receptors from binding to TGFβ3;
(d) the anti-TGFβ3 antibody directly contacts amino acid residue R394 in the beta6/beta7 hairpin region of human TGFβ3;
(e) the anti-TGFβ3 antibody directly contacts amino acid residue R394 in the beta6/beta7 hairpin region of human TGFβ3 and residue R394 of TGFβ3 makes an ionic salt bridge with the anti-TGFβ3 antibody in the heavy chain CDR2;
(f) isoform selectivity of the anti-TGFβ3 antibody for TGFβ3 over TGFβ1 is achieved by direct contact by the antigen binding domain of the anti-TGFβ3 antibody with amino acid residues T387, L389, and T395 of TGFβ3 (human TGFβ3 numbering);
(g) the isoform selectivity of the anti-TGFβ3 antibody for TGFβ3 over TGFβ2 is achieved by direct contact by the antigen binding domain of the anti-TGFβ3 antibody with amino acid residues R325, R394, and V398 of TGFβ3 (human TGFβ3 numbering);
(h) the anti-TGFβ3 antibody has reduced toxicity relative to the pan-TGFβ antibody 1D11;
(i) the anti-TGFβ3 antibody has reduced toxicity in rodents or cynomolgus monkeys relative to the pan-TGFβ antibody 1D11;
(j) the anti-TGFβ3 antibody has reduced toxicity relative to the pan-TGFβ small molecule inhibitor galunisertib;
(k) the anti-TGFβ3 antibody has reduced toxicity in rodents relative to the pan-TGFβ small molecule inhibitor galunisertib;
(l) the anti-TGFβ3 antibody has reduced toxicity relative to the anti-TGFβ1 antibody CAT-192;
(m) the anti-TGFβ3 antibody has reduced toxicity relative to an isoform selective anti-TGFβ2 antibody and/or anti-TGFβ2/3 antibody;
(n) the anti-TGFβ3 antibody comprises: (i) heavy chain CDRs comprising CDR-H1, -H2 and -H3, wherein CDR-H1 has the amino acid sequence of SEQ ID NO: 4, CDR-H2 has the amino acid sequence of one of SEQ ID NOs: 5, 34, 35, and 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9;
(o) the anti-TGFβ3 antibody comprises an antigen binding domain that directly contacts amino acid residues R325, K331, W332, H334, E335, T387, I388, L389, Y391, V392, G393, R394, P396, K397, and V398 on human TGFβ3; and
(p) the anti-TGFβ3 antibody as in (o), wherein the antigen binding domain is within 15-8, 8, 8-5, 7-5, 6-5, or 5 angstroms of the TGFβ3 amino acid residues.

Embodiment 2. An isolated anti-TGFβ3 antibody, wherein the antibody comprises: (i) heavy chain CDRs comprising CDR-H1, -H2 and -H3, wherein CDR-H1 has the amino acid sequence of SEQ ID NO: 4, CDR-H2 has the amino acid sequence of one of SEQ ID NOs: 5, 34, 35, and 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9.

Embodiment 3. The anti-TGFβ3 antibody of Embodiment 1 or Embodiment 2, wherein the antibody comprises a CDR-H2 having the amino acid sequence of SEQ ID NO: 5.

Embodiment 4. The anti-TGFβ3 antibody of any of Embodiments 1 to 3, wherein the antibody comprises a heavy chain variable region (VH) amino acid sequence having at least 95%, at least 96%, at least 97%, at least 98%, at least 99%, or 100% sequence identity to an amino acid sequence selected from the group consisting of SEQ ID NOs: 23, 37, and 42-50.

Embodiment 5. The anti-TGFβ3 antibody of any of Embodiments 1 to 4, wherein the antibody comprises a VH amino acid sequence selected from the group consisting of SEQ ID NOs: 23, 37, and 42-50.

Embodiment 6. The anti-TGFβ3 antibody of any of Embodiments 1 to 5, wherein the antibody comprises complete heavy (H) chain amino acid sequence having at least 95%, at least 96%, at least 97%, at least 98%, at least 99%, or 100% sequence identity to an amino acid sequence selected from the group consisting of SEQ ID NOs: 29, 59, 64, and 65-72. Embodiment 7. The anti-TGFβ3 antibody of any of Embodiments 1 to 6, wherein the antibody comprises complete heavy (H) chain amino acid sequence selected from the group consisting of SEQ ID NOs: 29, 59, 64, and 65-72.

Embodiment 8. The anti-TGFβ3 antibody of any of Embodiments 1 to 7, wherein the antibody comprises a light chain variable region (VL) amino acid sequence having at least 95%, at least 96%, at least 97%, at least 98%, at least 99%, or 100% sequence identity an amino acid sequence selected from the group consisting of SEQ ID NOs: 22, 36, and 38-41.

Embodiment 9. The anti-TGFβ3 antibody of any of Embodiments 1 to 8, wherein the antibody comprises a complete light (L) chain amino acid sequence having at least 95%, at least 96%, at least 97%, at least 98%, at least 99%, or 100% sequence identity to an amino acid sequence selected from the group consisting of SEQ ID NOs: 28, 58, and 60-63.

Embodiment 10. The anti-TGFβ3 antibody of any of Embodiments 1 to 9, wherein the antibody comprises a complete L chain amino acid sequence selected from the group consisting of SEQ ID NOs: 28, 58, and 60-63.

Embodiment 11. The anti-TGFβ3 antibody of any of Embodiments 1 to 10, wherein the antibody comprises a VH/VL pair, the VH/VL pair comprising amino acid sequences (respectively) selected from the group consisting of SEQ ID NOs: 23/22 (rat 2A10), SEQ ID NOs: 37/36 (vi), SEQ ID NOs: 37/38 (v1.1), SEQ ID NOs: 37/39 (v1.2), SEQ ID NOs: 37/40 (v1.3), SEQ ID NOs: 37/41 (v1.4), SEQ ID NOs: 42/36 (v1.5), SEQ ID NOs: 43/36 (v1.6), SEQ ID NOs:44/36 (v1.7), SEQ ID NOs: 45/36 (v2), SEQ ID NOs: 45/38 (v2.1), SEQ ID NOs: 45/39 (v2.2), SEQ ID NOs: 45/40 (v2.3), SEQ ID NOs: 45/41 (v2.4), SEQ ID NOs: 46/36 (v2.5), SEQ ID NOs: 47/36 (v2.6), SEQ ID NOs: 48/36 (v2.7), SEQ ID NOs: 49/36 (v2.8), and SEQ ID NOs: 50/36 (v2.9).

Embodiment 12. The anti-TGFβ3 antibody of any of Embodiments 1 to 11, wherein the antibody comprises a complete H/L chain pair, the complete H/L chain pair comprising amino acid sequences (respectively) selected from the group consisting of SEQ ID NOs: 29/28 (rat 2A10), SEQ ID NOs: 59/58 (v1), SEQ ID NOs: 59/60 (v1.1), SEQ ID NOs: 59/61 (v1.2), SEQ ID NOs: 59/62 (v1.3), SEQ ID NOs: 59/63 (v1.4), SEQ ID NOs: 64/58 (v1.5), SEQ ID NOs: 65/58 (v1.6), SEQ ID NOs: 66/58 (v1.7), SEQ ID NOs: 67/58 (v2), SEQ ID NOs: 67/60 (v2.1), SEQ ID NOs: 67/61 (v2.2), SEQ ID NOs: 67/62 (v2.3), SEQ ID NOs: 67/63 (v2.4), SEQ ID NOs: 68/58 (v2.5), SEQ ID NOs: 69/58 (v2.6), SEQ ID NOs: 70/58 (v2.7), SEQ ID NOs: 71/58 (v2.8), SEQ ID NOs: 72/58 (v2.9).

Embodiment 13. The anti-TGFβ3 antibody of any of Embodiments 1 to 10, wherein the antibody comprises a VL of SEQ ID NO: 22 comprising one or more framework modifications selected from the group consisting of: 4L or 4M, 38H or 38Q, 43A or 43Q, and 58V.

Embodiment 14. The anti-TGFβ3 antibody of any of Embodiments 1 to 10, wherein the VL comprises a set of framework modifications selected from the group consisting of: (i) 4L in FR1, 38H and 43Q in FR2, 58I in FR3 (h2A10.v1 and h2A10.v2);
(ii) 4M in FR1 (h2A10.v1.1 and h2A10.v2.1);
(iii) 38Q in FR2 (h2A10.v1.2 and h2A10.v2.2);
(iv) 43A in FR2 (h2A10.v1.3 and h2A10.v2.3);
(v) 58V in FR3 (h2A10.v1.4 and h2A10.v2.4);
(vi) 38Q, 43A in FR2, 58V in FR3 (h2A10.v3 and h2A10.v4);
(vii) 58V in FR3 (h2A10.v1.4 and h2A10.v2.4); and
(vi) 38Q, 43A in FR2, 58V in FR3 (h2A10.v3 and h2A10.v4).

Embodiment 15. The anti-TGFβ3 antibody of any of Embodiments 1 to 10, 13 and 14, wherein the antibody comprises a VH of SEQ ID NO: 23 comprising one or more framework modifications selected from the group consisting of: 47L or 47W; 49A, 49S, or 49G; 73D or 73N; and 76N, 78D or 78L, 78A, or 78V.

Embodiment 16. The anti-TGFβ3 antibody of Embodiment 15, wherein the VH comprises a set of framework modifications selected from the group consisting of:
(i) 47L, 49A in FR2, 78V in FR3 (h2A10.v1);
(ii) 47L, 49A in FR2, 73D, 76S, 78V in FR3 (h2A10.v2);
(iii) 47W in FR2 (h2A10.v1.5);
(iv): 49G in FR2 (h2A10.v1.6);
(v) 78A in FR3 (h2A10.v1.7);
(vi) 47W in FR2 (h2A10.v2.5);
(vii) 49S in FR2 (h2A10.v2.6);
(viii) 73N in FR3 (h2A10.v2.7);
(ix) 76N in FR3 (h2A10.v2.8);
(x) 78L in FR3 (h2A10.v2.9); and
(xi) 49S in FR2, 76N, 78L in FR3 (h2A10.v3 and h2A10.v4).

Embodiment 17. The antibody of Embodiment 13 or 15 comprising a VL, wherein the VL retains leucine (L) at position 4 in framework I and leucine (L) at position 47 in framework II.

Embodiment 18. The antibody of Embodiment 15 or 17, wherein the VH retains D at position 73 in framework III of VH from r2A10.

Embodiment 19. The anti-TGFβ3 antibody of Embodiment 1 or 2, wherein the anti-TGFβ3 antibody comprises a CDR-H2 having the amino acid sequence of SEQ ID NO: 159.

Embodiment 20. The anti-TGFβ3 antibody of Embodiment 19, wherein the antibody comprises a VH amino acid sequence having at least 95%, at least 96%, at least 97%, at least 98%, at least 99%, or 100% sequence identity to the amino acid sequence of SEQ ID NO: 52.

Embodiment 21. The anti-TGFβ3 antibody of Embodiment 19 or 20, wherein the antibody comprises the VH amino acid sequence of SEQ ID NO: 52.

Embodiment 22. The anti-TGFβ3 antibody of any of Embodiments 19 to 21, wherein the antibody comprises a complete H chain amino acid sequence having at least 95%, at least 96%, at least 97%, at least 98%, at least 99%, or 100% sequence identity to the amino acid sequence of SEQ ID NO: 74.

Embodiment 23. The anti-TGFβ3 antibody of any of Embodiments 19 to 22, wherein the antibody comprises the complete H chain amino acid sequence of SEQ ID NO: 74.

Embodiment 24. The anti-TGFβ3 antibody of any of Embodiments 19 to 23, wherein the antibody comprises a VL amino acid sequence having at least 95%, at least 96%, at least 97%, at least 98%, at least 99%, or 100% sequence identity to an amino acid sequence selected from the group consisting of SEQ ID NOs: 22, 36, and 38-41.

Embodiment 25. The anti-TGFβ3 antibody of any of Embodiments 19 to 24, wherein the antibody comprises a VL amino acid sequence selected from the group consisting of SEQ ID NOs: 22, 36, and 38-41.

Embodiment 26. The anti-TGFβ3 antibody of any of Embodiments 19 to 25, wherein the antibody comprises a complete L chain amino acid sequence having at least 95%, at least 96%, at least 97%, at least 98%, at least 99%, or 100% sequence identity to an amino acid sequence selected from the group consisting of SEQ ID NOs: 28, 58, and 60-63.

Embodiment 27. The anti-TGFβ3 antibody of any of Embodiments 19 to 26, wherein the antibody comprises a complete L chain amino acid sequence selected from the group consisting of SEQ ID NOs: 28, 58, and 60-63.

Embodiment 28. The anti-TGFβ3 antibody of Embodiment 19, wherein the antibody comprises a VH/VL pair comprising the amino acid sequences (respectively) of SEQ ID NOs: 52/36.

Embodiment 29. The anti-TGFβ3 antibody of Embodiment 19, wherein the antibody comprises a complete H/L chain pair, the H/L chain pair comprising the amino acid sequences (respectively) of SEQ ID NOs: 74/58.

Embodiment 30. The anti-TGFβ3 antibody of Embodiment 1 or 2, wherein the antibody comprises a CDR-H2 having the amino acid sequence of SEQ ID NO: 34.

Embodiment 31. The anti-TGFβ3 antibody of Embodiment 30, wherein the antibody comprises a VH amino acid sequence having at least 95%, at least 96%, at least 97%, at least 98%, at least 99%, or 100% sequence identity to the amino acid sequence of SEQ ID NO: 51 or 55.

Embodiment 32. The anti-TGFβ3 antibody of Embodiment 30 or 31, wherein the antibody comprises the VH amino acid sequence of SEQ ID NO: 51 or 55.

Embodiment 33. The anti-TGFβ3 antibody of any of Embodiments 30 to 32, wherein the antibody comprises a complete H chain amino acid sequence having at least 95%, at least 96%, at least 97%, at least 98%, at least 99%, or 100% sequence identity to the amino acid sequence of SEQ ID NO: 73 or 77.

Embodiment 34. The anti-TGFβ3 antibody of any of Embodiments 30 to 33, wherein the antibody comprises the complete H chain amino acid sequence of SEQ ID NO: 73.

Embodiment 35. The anti-TGFβ3 antibody of any of Embodiments 30 to 33, wherein the antibody comprises the complete H chain amino acid sequence of SEQ ID NO: 77.

Embodiment 36. The anti-TGFβ3 antibody of any of Embodiments 30 to 35 wherein the antibody comprises a VL amino acid sequence having at least 95%, at least 96%, at least 97%, at least 98%, at least 99%, or 100% sequence identity to an amino acid sequence selected from the group consisting of SEQ ID NOs: 22, 36, and 38-41.

Embodiment 37. The anti-TGFβ3 antibody of any of Embodiments 30 to 36, wherein the antibody comprises a VL amino acid sequence selected from the group consisting of SEQ ID NOs: 22, 36, and 38-41.

Embodiment 38. The anti-TGFβ3 antibody of any of Embodiments 30 to 37, wherein the antibody comprises a complete L chain amino acid sequence having at least 95%, at least 96%, at least 97%, at least 98%, at least 99%, or 100% sequence identity to an amino acid sequence selected from the group consisting of SEQ ID NOs: 28, 58, and 60-63.

Embodiment 39. The anti-TGFβ3 antibody of any one of Embodiments 30 to 38, wherein the antibody comprises a complete L chain amino acid sequence selected from the group consisting of SEQ ID NOs: 28, 58, and 60-63.

Embodiment 40. The anti-TGFβ3 antibody of Embodiment 30, wherein the antibody comprises a VH/VL pair, the VH/VL pair comprising the amino acid sequences (respectively) of SEQ ID NOs: 51/36 or SEQ ID NOs: 55/54.

Embodiment 41. The anti-TGFβ3 antibody of Embodiment 30, wherein the antibody comprises a complete H/L chain pair, the H/L chain pair comprising the amino acid sequences (respectively) of SEQ ID NOs: 73/58 or SEQ ID NOs: 77/76.

Embodiment 42. The anti-TGFβ3 antibody of Embodiment 1 or 2, wherein the antibody comprises a CDR-H2 having the amino acid sequence of SEQ ID NO: 35.

Embodiment 43. The anti-TGFβ3 antibody of Embodiment 42, wherein the antibody comprises a VH amino acid sequence having at least 95%, at least 96%, at least 97%, at least 98%, at least 99%, or 100% sequence identity to the amino acid sequence of SEQ ID NO: 53 or 57.

Embodiment 44. The anti-TGFβ3 antibody of Embodiment 42 or 43, wherein the antibody comprises the VH amino acid sequence of SEQ ID NO: 53.

Embodiment 45. The anti-TGFβ3 antibody of Embodiment 42 or 43, wherein the antibody comprises the VH amino acid sequence of SEQ ID NO: 57.

Embodiment 46. The anti-TGFβ3 antibody of any of Embodiments 42 to 45, wherein the antibody comprises a complete H chain amino acid sequence having at least 95%, at least 96%, at least 97%, at least 98%, at least 99%, or 100% sequence identity to the amino acid sequence of SEQ ID NO: 75 or 79.

Embodiment 47. The anti-TGFβ3 antibody of any of Embodiments 42 to 46, wherein the antibody comprises the complete H chain amino acid sequence of SEQ ID NO: 75.

Embodiment 48. The anti-TGFβ3 antibody of any of Embodiments 42 to 46, wherein the antibody comprises a complete H chain amino acid sequence of SEQ ID NO: 79.

Embodiment 49. The anti-TGFβ3 antibody of any of Embodiments 42 to 48, wherein the antibody comprises a VL amino acid sequence having at least 95%, at least 96%, at least 97%, at least 98%, at least 99%, or 100% sequence identity to an amino acid sequence selected from the group consisting of SEQ ID NOs: 22, 36, and 38-41.

Embodiment 50. The anti-TGFβ3 antibody of any one of Embodiments 42 to 49, wherein the antibody comprises a VL amino acid sequence selected from the group consisting of SEQ ID NOs: 22, 36, and 38-41.

Embodiment 51. The anti-TGFβ3 antibody of any of Embodiments 42 to 50, wherein the antibody comprises a complete L chain amino acid sequence having at least 95%, at least 96%, at least 97%, at least 98%, at least 99%, or 100% sequence identity to the amino acid sequence selected from the group consisting of SEQ ID NOs: 28, 58, and 60-63.

Embodiment 52. The anti-TGFβ3 antibody of any of Embodiments 42 to 51, wherein the antibody comprises a complete L chain amino acid sequence selected from the group consisting of SEQ ID NOs: 28, 58, and 60-63.

Embodiment 53. The anti-TGFβ3 antibody of Embodiment 42, wherein the antibody comprises a VH/VL pair comprising the amino acid sequences (respectively) of SEQ ID NOs: 53/36 or SEQ ID NOs: 57/56.

Embodiment 54. The anti-TGFβ3 antibody of Embodiment 42, wherein the antibody comprises a complete H/L chain pair, the H/L chain pair comprising the amino acid sequences (respectively) of SEQ ID NOs: 75/58 or SEQ ID NOs: 79/78.

Embodiment 55. An anti-TGFβ3 antibody comprising:
(a) (i) heavy chain CDRs comprising CDR-H1, -H2 and -H3, wherein CDR-H1 has the amino acid sequence of SEQ ID NO: 4, CDR-H2 has the amino acid sequence of one of SEQ ID NO: 35, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9;
(b) a VH/VL pair, the VH of the VH/VL pair comprising the amino acid sequence of SEQ ID NO: 57, and the VL of the VH/VL pair comprising the amino acid sequence of SEQ ID NO: 56; or
(c) a complete H/L chain pair, the H chain of the H/L chain pair comprising the amino acid sequence of SEQ ID NO: 79 and the L chain of the H/L chain pair comprising the amino acid sequence of SEQ ID NO: 78.

Embodiment 56. An isolated anti-TGFβ2/3 antibody, wherein the antibody selectively neutralizes TGFβ2 and TGFβ3, and wherein the antibody comprises one or more of the following features:
(a) selectivity of the anti-TGFβ2/3 antibody for TGFβ2 and TGFβ3 over human TGFβ1, with respect to selective neutralization, is achieved by direct contact of the antibody's antigen binding domain with amino acid residue E373 TGFβ2 or TGFβ3 (human TGFβ2 numbering);
(b) the anti-TGFβ2/3 antibody neutralizes TGFβ2 and/or TGFβ3 via an allosteric mechanism;
(c) the anti-TGFβ2/3 antibody induces a conformational change in TGFβ2 and/or TGFβ3 homodimer;
(d) the anti-TGFβ2/3 antibody induces a conformational change in TGFβ2 and/or TGFβ3 homodimer, wherein the conformational change comprises the two monomers pinching together by several degrees;
(e) the anti-TGFβ2/3 antibody is a divalent antibody or a monovalent antibody;
(f) the anti-TGFβ2/3 antibody comprises (i) heavy chain CDRs comprising CDR-H1, -H2 and -H3, wherein CDR-H1 has the amino acid sequence of SEQ ID NO: 10, CDR-H2 has the amino acid sequence of SEQ ID NO: 11, and CDR-H3 has the amino acid sequence of SEQ ID NO: 12; and (ii) light chain CDRs comprising CDR-L1, -L2 and -L3, wherein CDR-L1 has the amino acid sequence of SEQ ID NO: 13; CDR-L2 has the amino acid sequence of SEQ ID NO: 14, and CDR-L3 has the amino acid sequence of SEQ ID NO: 15;
(g) the anti-TGFβ2/3 antibody specifically binds to TGFβ2 homodimer, the TGFβ2 homodimer having a first and a second TGFβ2 monomer, and wherein the anti-TGFβ2/3 antibody comprises an antigen binding domain that directly contacts (i) amino acid residues V313, Q314, D315, R320, L322, Y323, R328, D329, F345, and A347 of the first TGFβ2 monomer, and (ii) amino acid residues N368, T369, I370, N371, P372, E373, A374, S375, A376, and S377 of the second TGFβ2 monomer (human TGFβ2 numbering);
(h) the anti-TGFβ2/3 antibody as in (g), wherein the antigen binding domain is within 5 angstroms of the TGFβ2 and/or TGFβ3 amino acid residues;
(i) wherein the anti-TGFβ2/3 antibody specifically binds to the same epitope on TGFβ3 as in (g); and
(j) the anti-TGFβ2/3 antibody does not neutralize TGFβ2 and/or TGFβ3 in monovalent form.

Embodiment 57. The anti-TGFβ2/3 antibody of Embodiment 56, wherein the antibody comprises a VH amino acid sequence having at least 95%, at least 96%, at least 97%, at least 98%, at least 99%, or 100% sequence identity to an amino acid sequence selected from the group consisting of SEQ ID NOs: 27, 81, 83, 86-88, 93-100, and 102-105.

Embodiment 58. The anti-TGFβ2/3 antibody of Embodiment 56 or 57, wherein the antibody comprises a VH amino acid sequence selected from the group consisting of SEQ ID NOs: 27, 81, 83, 86-88, 93-100, and 102-105.

Embodiment 59. The anti-TGFβ2/3 antibody of any of Embodiments 56 to 58, wherein the antibody comprises a complete H chain amino acid sequence having at least 95%, at least 96%, at least 97%, at least 98%, at least 99%, or 100% sequence identity to the amino acid sequence selected from the group consisting of SEQ ID NOs: 33, 107, 109, 112-114, and 119-130.

Embodiment 60. The anti-TGFβ2/3 antibody of any of Embodiments 56 to 59, wherein the antibody comprises a VL amino acid sequence having at least 95%, at least 96%, at least 97%, at least 98%, at least 99%, or 100% sequence identity to the amino acid sequence selected from the group consisting of SEQ ID NOs: 26, 80, 82, 84, 85, 89-92, and 101.

Embodiment 61. The anti-TGFβ2/3 antibody of any of Embodiments 56 to 60, wherein the antibody comprises a VL amino acid sequence selected from the group consisting of SEQ ID NOs: 26, 80, 82, 84, 85, 89-92, and 101.

Embodiment 62. The anti-TGFβ2/3 antibody of any of Embodiments 56 to 61, wherein the antibody comprises a complete L chain amino acid sequence having at least 95%, at least 96%, at least 97%, at least 98%, at least 99%, or 100% sequence identity to an amino acid sequence selected from the group consisting of SEQ ID NOs: 32, 106, 108, 110, 111, 115-118, and 186.

Embodiment 63. The anti-TGFβ2/3 antibody of any of Embodiments 56 to 62, wherein the antibody comprises a complete L chain amino acid sequence selected from the group consisting of SEQ ID NOs: 32, 106, 108, 110, 111, 115-118, and 186.

Embodiment 64. The anti-TGFβ2/3 antibody of Embodiment 56, wherein the antibody comprises a VH/VL pair, the VH/VL pair comprising amino acid sequences (respectively) selected from the group consisting of SEQ ID NOs: 27/26 (rabbit 4A11), SEQ ID NOs: 81/80 (v1), SEQ ID NOs: 81/82 (v2), SEQ ID NOs: 83/80 (v3), SEQ ID NOs: 83/82 (v4), SEQ ID NOs: 81/84 (v5), SEQ ID NOs: 81/85 (v6), SEQ ID NOs: 83/84 (v7), SEQ ID NOs: 86/84 (v7.1), SEQ ID NOs: 87/84 (v7.2), SEQ ID NOs: 88/84 (v7.3), SEQ ID NOs: 83/89 (v7.4), SEQ ID NOs: 83/90 (v7.5), SEQ ID NOs: 83/91 (v7.6), SEQ ID NOs: 83/92 (v7.7), SEQ ID NOs: 93/84 (v7.8), SEQ ID NOs: 94/84 (v7.9), SEQ ID NOs: 95/84 (v7.10), SEQ ID NOs: 96/84 (v7.11), SEQ ID NOs: 97/84 (v7.12), SEQ ID NOs: 98/84 (v7.13), SEQ ID NOs: 99/84 (v7.14), SEQ ID NOs: 100/84 (v7.15), SEQ ID NOs: 102/101 (v7.16), SEQ ID NOs: 103/101 (v7.17), SEQ ID NOs: 104/101 (v7.18), SEQ ID NOs: 105/101 (v7.19), and SEQ ID NOs: 83/85 (v8).

Embodiment 65. The anti-TGFβ2/3 antibody of Embodiment 56, wherein the antibody comprises a complete H/L chain pair, the complete H/L chain pair comprising amino acid sequences (respectively) selected from the group consisting of SEQ ID NOs: 32/33 (rabbit 4A11), SEQ ID NOs: 107/106 (v1), SEQ ID NOs: 107/108 (v2), SEQ ID NOs: 109/106 (v3), SEQ ID NOs: 109/108 (v4), SEQ ID NOs: 107/110 (v5), SEQ ID NOs: 107/111 (v6), SEQ ID NOs: 109/110 (v7), SEQ ID NOs: 112/110 (v7.1), SEQ ID NOs: 113/110 (v7.2), SEQ ID NOs: 114/110 (v7.3), SEQ ID NOs: 114/115 (v7.4), SEQ ID NOs: 114/116 (v7.5), SEQ ID NOs: 114/117 (v7.6), SEQ ID NOs: 114/118 (v7.7), SEQ ID NOs: 119/110 (v7.8), SEQ ID NOs:120/110 (v7.9), SEQ ID NOs: 121/110 (v7.10), SEQ ID NOs: 122/110 (v7.11), SEQ ID NOs: 123/110 (v7.12), SEQ ID NOs: 124/110 (v7.13), SEQ ID NOs: 125/110 (v7.14), SEQ ID NOs: 126/110 (v7.15), SEQ ID NOs: 127/186 (v7.16), SEQ ID NOs: 128/186 (v7.17), SEQ ID NOs: 129/186 (v7.18), SEQ ID NOs: 130/186 (v7.19), and SEQ ID NOs: 114/111 (v8).

Embodiment 66. The anti-TGFβ2/3 antibody of Embodiment 56, wherein the antibody comprises a VL comprising the amino acid sequence of SEQ ID NO: 26 comprising one or more framework modifications selected from the group consisting of: 2A or 2I, 4L, 36F or 36Y, 43P or 43A, and 58V or 58I.

Embodiment 67. The anti-TGFβ2/3 antibody of Embodiment 56, wherein the VL comprises a set of framework modifications selected from the group consisting of:
(i) 2A and 4L in FR1 and 36F in FR2 (h4A11.v1 and h4A11.v3);
(ii) 2A and 4L in FR1 and 36F and 43P in FR2 (h4A11.v2 and h4A11.v4);
(iii) 2A in FR1, 36F and 43P in FR2 and 58V in FR3 (h4A11.v5 and h4A11.v7);
(iv) 2A and 4L in FR1 and 36F in FR2 (h4A11.v6 and h4A11.v8);
(v) 2I in FR1 (h4A11.v7.4);
(vi) 36Y in FR2 (h4A11.v7.5);
(vii) 43A in FR2 (h4A11.v7.6);
(viii) 58I in FR3 (h4A11.v7.7); and
(ix) 2I in FR1, 43A in FR2, 58I in FR3 (h4A11.v7.16-19).

Embodiment 68. The anti-TGFβ2/3 antibody of Embodiment 56, wherein the antibody comprises a VH comprising the amino acid sequence of SEQ ID NO: 27 comprising one or more framework modifications selected from the group consisting of: deletion of 1E, 2Q or 2V, 24V, 37V or 37I, 48I, 49G, 67F or 67V, 71K or 71V, 73S or 73T, deletion of 75K and 76N, 78V or 78F, 91F or 91Y, 105P or 105Q.

Embodiment 69. The anti-TGFβ2/3 antibody of Embodiment 56 or 68, wherein the VL comprises a set of framework modifications selected from the group consisting of:
(i) 2Q and 24V in FR1, 48I and 49G in FR2, 71K, 73S, 78V and 91F in FR3 and 105P in FR4 (h4A11.v1, h4A11.v2, h4A11.v5, h4A11.v6);
(ii) 2Q in FR1, 37V in FR2, 67F, 71K, 73S, 78V and 91F in FR3 and 105P in FR4 (h4A11.v3, h4A11.v4, h4A11.v7, h4A11.v8);
(iii) delete 1E in FR1 (h4A11.v7.1);
(iv) delete 75K and 76N in FR3 (h4A11.v7.2);
(v) delete 1E in FR1 and 75K76N in FR3 (h4A11.v7.3);
(vi) 2V in FR1 (h4A11.v7.8);
(vi) 37I in FR2 (h4A11.v7.9);
(vii) 67V in FR3 (h4A11.v7.10);
(viii) 71V in FR3 (h4A11.v7.11);
(ix) 73T in FR3 (h4A11.v7.12);
(x) 78F in FR3 (h4A11.v7.13);
(xi) 91Y in FR3 (h4A11.v7.14);
(xii) 105Q in FR4 (h4A11.v7.15);
(xiii) 2V in FR1, 37I in FR2, 67V, 73T, 78F in FR3, 105Q in FR4 ((h4A11.v7.16); (xiv) 2V in FR1, 37I in FR2, 67V, 73T, 91Y in FR3, 105Q in FR4 (h4A11.v7.17);
(xv) 2V in FR1, 37I in FR2, 67V, 73T in FR3, 105Q in FR4 (h4A11.v7.18); and
(xvi) 2V in FR1, 37I in FR2, 67V, 73T, deletion of 75K and 76N in FR3, 105Q in FR4 (h4A11.v7.19).

Embodiment 70. An isolated anti-TGFβ2 antibody, wherein the antibody comprises:
(a) heavy chain CDRs comprising CDR-H1, -H2 and -H3, wherein CDR-H1 has the amino acid sequence of SEQ ID NO: 16, CDR-H2 has the amino acid sequence of SEQ ID NO: 17, and CDR-H3 has the amino acid sequence of SEQ ID NO: 18; and
(b) light chain CDRs comprising CDR-L1, -L2 and -L3, wherein CDR-L1 has the amino acid sequence of SEQ ID NO: 19; CDR-L2 has the amino acid sequence of SEQ ID NO: 20, and CDR-L3 has the amino acid sequence of SEQ ID NO: 21.

Embodiment 71. The anti-TGFβ2 antibody of Embodiment 70, wherein the antibody selectively neutralizes TGFβ2.

Embodiment 72. The anti-TGFβ2 antibody of Embodiment 71, wherein the antibody:
(a) has reduced toxicity relative to the pan-TGFβ antibody 1D11;
(b) has reduced toxicity in rodents relative to the pan-TGFβ antibody 1D11;
(c) has reduced toxicity relative to the pan-TGFβ small molecule inhibitor galunisertib; and/or
(d) has reduced toxicity in rodents relative to the pan-TGFβ small molecule inhibitor galunisertib.

Embodiment 73. The anti-TGFβ2 antibody of Embodiment 70 or 71, wherein the antibody comprises a VH amino acid sequence having at least 95%, at least 96%, at least 97%, at least 98%, at least 99%, or 100% sequence identity to an amino acid sequence selected from the group consisting of SEQ ID NOs: 25, 132, and 138-142.

Embodiment 74. The anti-TGFβ2 antibody of Embodiment 73, wherein the antibody comprises a VH amino acid sequence selected from the group consisting of SEQ ID NOs: 25, 132, and 138-142.

Embodiment 75. The anti-TGFβ2 antibody of Embodiment 70 or 71, wherein the antibody comprises a complete H chain amino acid sequence having at least 95%, at least 96%, at least 97%, at least 98%, at least 99%, or 100% sequence identity to the amino acid sequence selected from the group consisting of SEQ ID NOs: 31, 146, and 152-156.

Embodiment 76. The anti-TGFβ2 antibody of Embodiment 75, wherein the antibody comprises a complete H chain amino acid sequence selected from the group consisting of SEQ ID NOs: 31, 146, and 152-156.

Embodiment 77. The anti-TGFβ2 antibody of Embodiment 70 or 71, wherein the antibody comprises a VL amino acid sequence having at least 95%, at least 96%, at least 97%, at least 98%, at least 99%, or 100% sequence identity to the amino acid sequence selected from the group consisting of SEQ ID NOs: 24, 131, 133-137, 143, and 144.

Embodiment 78. The anti-TGFβ2 antibody of Embodiment 77, wherein the antibody comprises a VL amino acid sequence selected from the group consisting of SEQ ID NOs: 24, 131, 133-137, 143, and 144.

Embodiment 79. The anti-TGFβ2 antibody of Embodiment 70 or 71, wherein the antibody comprises a complete L chain amino acid sequence having at least 95%, at least 96%, at least 97%, at least 98%, at least 99%, or 100% sequence identity to an amino acid sequence selected from the group consisting of SEQ ID NOs: 30, 145, 147-151, 157, and 158.

Embodiment 80. The anti-TGFβ2 antibody of Embodiment 79, wherein the antibody comprises a complete L chain amino acid sequence selected from the group consisting of SEQ ID NOs: 30, 145, 147-151, 157, and 158.

Embodiment 81. The anti-TGFβ2 antibody of Embodiment 70 or 71, wherein the antibody comprises a VH/VL pair, the VH/VL pair comprising amino acid sequences selected from the group consisting of SEQ ID NOs: 25/24 (rabbit 6F12), SEQ ID NOs: 132/131 (v1), SEQ ID NOs: 132/133 (v1.1), SEQ ID NOs: 132/134 (v1.2), SEQ ID NOs: 132/135 (v1.3), SEQ ID NOs: 132/136 (v1.4), SEQ ID NOs: 132/137 (v1.5), SEQ ID NOs: 138/131 (v1.6), SEQ ID NOs: 139/131 (v1.7), SEQ ID NOs: 140/131 (v1.8), SEQ ID NOs: 141/131 (v1.9), SEQ ID NOs: 142/131 (v2), SEQ ID NOs: 132/143 (v3), and SEQ ID NOs: 142/144 (v4).

Embodiment 82. The anti-TGFβ2 antibody of Embodiment 70 or 71, wherein the antibody comprises a complete H/L chain pair, the complete H/L chain pair comprising amino acid sequences selected from the group consisting of SEQ ID NOs: 31/30 (rabbit 6F12), SEQ ID NOs: 146/145 (v1), SEQ ID NOs: 146/147 (v1.1), SEQ ID NOs: 146/148 (v1.2), SEQ ID NOs: 146/149 (v1.3), SEQ ID NOs: 146/150 (v1.4), SEQ ID NOs: 146/151 (v1.5), SEQ ID NOs: 152/145 (v1.6), SEQ ID NOs: 153/145 (v1.7), SEQ ID NOs: 154/145 (v1.8), SEQ ID NOs: 155/145 (v1.9), SEQ ID NOs: 156/145 (v2), SEQ ID NOs: 146/157 (v3), and SEQ ID NOs: 156/158 (v4).

Embodiment 83. The anti-TGFβ2 antibody of Embodiment 70 or 71, wherein the antibody comprises a VL comprising the amino acid sequence of SEQ ID NO: 24 comprising one or more framework mutations selected from the group consisting of 43S or 43A, 66G, 69T, 71F, and 87Y.

Embodiment 84. The anti-TGFβ2 antibody of Embodiment 83, wherein the VL comprises a set of framework mutations selected from the group consisting of:
(i) 43 S in FR2 and 66E, 69P, 71Y and 87F in FR3 (h6F12.v1 and h6F12.v2);
(ii) 43S in FR2 and 58V, 66E, 69P, 71Y and 87F in FR3 (h6F12.v3 and h6F12.v4); (iii) 43A in FR2 (h6F12.v1.1);
(iv) 66G in FR3 (h6F12.v1.2);
(v) 69T in FR3 (h6F12.v1.3);
(vi) 71F in FR3 (h6F12.v1.4); and
(vii) 87Y in FR3 (h6F12.v1.5).

Embodiment 85. The anti-TGFβ2 antibody of Embodiment 70 or 71, wherein the antibody comprises a VH comprising the amino acid sequence of SEQ ID NO: 25 comprising one or more framework mutations selected from the group consisting of 37V or 37I, 48M or 48L, 49G or 49A, 67L, 71K and 78V, and 105P or 105R.

Embodiment 86. The anti-TGFβ2 antibody of Embodiment 85, wherein the VH comprises a set of framework mutations selected from the group consisting of:
(i) 37V, 48M and 49G in FR2 and 105P in FR4 (h6F12.v1 and h6F12.v3);
(ii) 37V and 48M in FR2, 67L, 71K and 78V in FR3 and 105P in FR4 (h6F12.v2 and h6F12.v4);
(iii) 37I in FR2 (h6F12.v1.6);
(iv) 48L in FR2 (h6F12.v1.7);
(v) 49A in FR2 (h6F12.v1.8);
(vi) 105R in FR4 (h6F12.v1.9);
(vii) 37V, 48M and 49G in FR2 and 105P in FR4 (h6F12.v1 and h6F12.v3);
(viii) 37V and 48M in FR2, 67L, 71K and 78V in FR3 and 105P in FR4 (6F12.v2 and h6F12.v4);
(ix) 37I in FR2 (h6F12.v1.6);
(x) 48L in FR2 (h6F12.v1.7);
(xi): 49A in FR2 (h6F12.v1.8); and
(xii) 105R in FR4 (h6F12.v1.9).

Embodiment 87. The anti-TGFβ3 antibody of any one of Embodiments 1 to 55, or the anti-TGFβ2/3 antibody of any one of Embodiments 56 to 68, wherein the antibody specifically binds to human TGFβ3.

Embodiment 88. The anti-TGFβ3 antibody of any one of Embodiments 1 to 55, wherein the antibody specifically binds to both the immature and mature forms of TGFβ3.

Embodiment 89. The anti-TGFβ2/3 antibody of any one of Embodiments 56 to 68, or the anti-TGFβ2 antibody of any one of Embodiments 70 to 83, wherein the antibody specifically binds to human TGFβ2.

Embodiment I-1. An isolated anti-tumor necrosis factor beta 3 (TGFβ3) antibody, wherein the antibody selectively neutralizes TGFβ3, and wherein the antibody comprises one or more of the following features:
(n) the anti-TGFβ3 antibody comprises: (i) heavy chain CDRs comprising CDR-H1, -H2 and -H3, wherein CDR-H1 has the amino acid sequence of SEQ ID NO: 4, CDR-H2 has the amino acid sequence of one of SEQ ID NOs: 5, 34, 35, and 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9;
(a) the anti-TGFβ3 antibody specifically binds to the beta6/beta7 hairpin region of TGFβ3;
(b) binding of the anti-TGFβ3 antibody sterically blocks the ability of TGFBR2, but not TGFBR1, to bind TGFβ3;
(c) binding of the anti-TGFβ3 antibody to TGFβ3 blocks TGFBR2 binding and inhibits the TGFBR1/TGFBR2 signaling receptors from binding to TGFβ3;
(d) the anti-TGFβ3 antibody directly contacts amino acid residue R394 in the beta6/beta7 hairpin region of human TGFβ3;
(e) the anti-TGFβ3 antibody directly contacts amino acid residue R394 in the beta6/beta7 hairpin region of human TGFβ3 and residue R394 of TGFβ3 makes an ionic salt bridge with the anti-TGFβ3 antibody in the heavy chain CDR2;
(f) isoform selectivity of the anti-TGFβ3 antibody for TGFβ3 over TGFβ1 is achieved by direct contact by the antigen binding domain of the anti-TGFβ3 antibody with amino acid residues T387, L389, and T395 of TGFβ3 (human TGFβ3 numbering);
(g) the isoform selectivity of the anti-TGFβ3 antibody for TGFβ3 over TGFβ2 is achieved by direct contact by the antigen binding domain of the anti-TGFβ3 antibody with amino acid residues R325, R394, and V398 of TGFβ3 (human TGFβ3 numbering);
(h) the anti-TGFβ3 antibody has reduced toxicity relative to the pan-TGFβ antibody 1D11;
(i) the anti-TGFβ3 antibody has reduced toxicity in rodents or cynomolgus monkeys relative to the pan-TGFβ antibody 1D11;
(j) the anti-TGFβ3 antibody has reduced toxicity relative to the pan-TGFβ small molecule inhibitor galunisertib;
(k) the anti-TGFβ3 antibody has reduced toxicity in rodents relative to the pan-TGFβ small molecule inhibitor galunisertib;
(l) the anti-TGFβ3 antibody has reduced toxicity relative to the anti-TGFβ1 antibody CAT-192;
(m) the anti-TGFβ3 antibody has reduced toxicity relative to an isoform selective anti-TGFβ2 antibody and/or anti-TGFβ2/3 antibody;
(o) the anti-TGFβ3 antibody comprises an antigen binding domain that directly contacts amino acid residues R325, K331, W332, H334, E335, T387, I388, L389, Y391, V392, G393, R394, P396, K397, and V398 on human TGFβ3; and
(p) the anti-TGFβ3 antibody as in (o), wherein the antigen binding domain is within 15-8, 8, 8-5, 7-5, 6-5, or 5 angstroms of the TGFβ3 amino acid residues.

Embodiment I-2. An isolated anti-TGFβ3 antibody, wherein the antibody comprises: (i) heavy chain CDRs comprising CDR-H1, -H2 and -H3, wherein CDR-H1 has the amino acid sequence of SEQ ID NO: 4, CDR-H2 has the amino acid sequence of one of SEQ ID NOs: 5, 34, 35, and 159, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9.

Embodiment I-3. The anti-TGFβ3 antibody of Embodiment I-1 or Embodiment I-2, wherein the antibody comprises a heavy chain variable region (VH) amino acid sequence having at least 95%, at least 96%, at least 97%, at least 98%, at least 99%, or 100% sequence identity to an amino acid sequence selected from the group consisting of SEQ ID NOs: 23, 37, 42-53, 55 and 57.

Embodiment I-4. The anti-TGFβ3 antibody of any of Embodiment I-1 to Embodiment I-3, wherein the antibody comprises a VH amino acid sequence selected from the group consisting of SEQ ID NOs: 23, 37, 42-53, 55 and 57.

Embodiment I-5. The anti-TGFβ3 antibody of any of Embodiment I-1 to Embodiment I-4, wherein the antibody comprises complete heavy (H) chain amino acid sequence having at least 95%, at least 96%, at least 97%, at least 98%, at least 99%, or 100% sequence identity to an amino acid sequence selected from the group consisting of SEQ ID NOs: 29, 59, 64, 65-75, 77, and 79.

Embodiment I-6. The anti-TGFβ3 antibody of any of Embodiment I-1 to Embodiment I-5, wherein the antibody comprises complete heavy (H) chain amino acid sequence selected from the group consisting of SEQ ID NOs: 29, 59, 64, 65-75, 77 and 79.

Embodiment I-7. The anti-TGFβ3 antibody of any of Embodiment I-1 to Embodiment I-6, wherein the antibody comprises a light chain variable region (VL) amino acid sequence having at least 95%, at least 96%, at least 97%, at least 98%, at least 99%, or 100% sequence identity an amino acid sequence selected from the group consisting of SEQ ID NOs: 22, 36, 38-41, 54, and 56.

Embodiment I-8. The anti-TGFβ3 antibody of any of Embodiment I-1 to Embodiment I-7, wherein the antibody comprises a VL amino acid sequence selected from the group consisting of SEQ ID NOs: 22, 36, 38-41, 54, and 56.

Embodiment I-9. The anti-TGFβ3 antibody of any of Embodiment I-1 to Embodiment I-8, wherein the antibody comprises a complete light (L) chain amino acid sequence having at least 95%, at least 96%, at least 97%, at least 98%, at least 99%, or 100% sequence identity to an amino acid sequence selected from the group consisting of SEQ ID NOs: 28, 58, 60-63, 76, and 78.

Embodiment I-10. The anti-TGFβ3 antibody of any of Embodiment I-1 to Embodiment I-9, wherein the antibody comprises a complete L chain amino acid sequence selected from the group consisting of SEQ ID NOs: 28, 58, and 60-63, 76, and 78.

Embodiment I-11. The anti-TGFβ3 antibody of any of Embodiment I-1 to Embodiment I-10, wherein the antibody comprises a VH/VL pair, the VH/VL pair comprising amino acid sequences (respectively) selected from the group consisting of SEQ ID NOs: 23/22 (rat 2A10), SEQ ID NOs: 37/36 (v1), SEQ ID NOs: 37/38 (v1.1), SEQ ID NOs: 37/39 (v1.2), SEQ ID NOs: 37/40 (v1.3), SEQ ID NOs: 37/41 (v1.4), SEQ ID NOs: 42/36 (v1.5), SEQ ID NOs: 43/36 (v1.6), SEQ ID NOs:44/36 (v1.7), SEQ ID NOs: 45/36 (v2), SEQ ID NOs: 45/38 (v2.1), SEQ ID NOs: 45/39 (v2.2), SEQ ID NOs: 45/40 (v2.3), SEQ ID NOs: 45/41 (v2.4), SEQ ID NOs: 46/36 (v2.5), SEQ ID NOs: 47/36 (v2.6), SEQ ID NOs: 48/36 (v2.7), SEQ ID NOs: 49/36 (v2.8), SEQ ID NOs: 50/36 (v2.9), SEQ ID NOs: 51/36 (h2A10.v2.N54S), SEQ ID NOs: 52/36 (h2A10.v2.N54Q), SEQ ID NOs: 53/36 (h2A10.v2.T56A), SEQ ID NOs: 55/54 (v3), and SEQ ID NOs: 57/56 (v4).

Embodiment I-12. The anti-TGFβ3 antibody of any of Embodiment I-1 to Embodiment I-11, wherein the antibody comprises a complete H/L chain pair, the complete H/L chain pair comprising amino acid sequences (respectively) selected from the group consisting of SEQ ID NOs: 29/28 (rat 2A10), SEQ ID NOs: 59/58 (v1), SEQ ID NOs: 59/60 (v1.1), SEQ ID NOs: 59/61 (v1.2), SEQ ID NOs: 59/62 (v1.3), SEQ ID NOs: 59/63 (v1.4), SEQ ID NOs: 64/58 (v1.5), SEQ ID NOs: 65/58 (v1.6), SEQ ID NOs: 66/58 (v1.7), SEQ ID NOs: 67/58 (v2), SEQ ID NOs: 67/60 (v2.1), SEQ ID NOs: 67/61 (v2.2), SEQ ID NOs: 67/62 (v2.3), SEQ ID NOs: 67/63 (v2.4), SEQ ID NOs: 68/58 (v2.5), SEQ ID NOs: 69/58 (v2.6), SEQ ID NOs: 70/58 (v2.7), SEQ ID NOs: 71/58 (v2.8), SEQ ID NOs: 72/58 (v2.9), SEQ ID NOs: 73/58 (h2A10.v2.N54S), SEQ ID NOs: 74/58 (h2A10.v2.N54Q), SEQ ID NOs: 75/58 (h2A10.v2.T56A), SEQ ID NOs: 77/76 (v3), and SEQ ID NOs: 79/78 (v4).

Embodiment I-13. The anti-TGFβ3 antibody of any of Embodiment I-1 to Embodiment I-10, wherein the antibody comprises a VL of SEQ ID NO: 22 comprising one or more framework modifications selected from the group consisting of: 4L or 4M, 38H or 38Q, 43A or 43Q, and 58V.

Embodiment I-14. The anti-TGFβ3 antibody of any of Embodiment I-1 to Embodiment I-10, wherein the VL comprises a set of framework modifications selected from the group consisting of:
(i) 4L in FR1, 38H and 43Q in FR2, 58I in FR3 (h2A10.v1 and h2A10.v2);
(ii) 4M in FR1 (h2A10.v1.1 and h2A10.v2.1);
(iii) 38Q in FR2 (h2A10.v1.2 and h2A10.v2.2);
(iv) 43A in FR2 (h2A10.v1.3 and h2A10.v2.3);
(v) 58V in FR3 (h2A10.v1.4 and h2A10.v2.4);
(vi) 38Q, 43A in FR2, 58V in FR3 (h2A10.v3 and h2A10.v4);
(vii) 58V in FR3 (h2A10.v1.4 and h2A10.v2.4); and
(vi) 38Q, 43A in FR2, 58V in FR3 (h2A10.v3 and h2A10.v4).

Embodiment I-15. The anti-TGFβ3 antibody of any of Embodiment I-1 to Embodiment I-10, Embodiment I-13, and Embodiment I-14, wherein the antibody comprises a VH of SEQ ID NO: 23 comprising one or more framework modifications selected from the group consisting of: 47L or 47W; 49A, 49S, or 49G; 73D or 73N; and 76N, 78D or 78L, 78A, or 78V.

Embodiment I-16. The anti-TGFβ3 antibody of Embodiment I-15, wherein the VH comprises a set of framework modifications selected from the group consisting of:
(i) 47L, 49A in FR2, 78V in FR3 (h2A10.v1);
(ii) 47L, 49A in FR2, 73D, 76S, 78V in FR3 (h2A10.v2);
(iii) 47W in FR2 (h2A10.v1.5);
(iv): 49G in FR2 (h2A10.v1.6);
(v) 78A in FR3 (h2A10.v1.7);
(vi) 47W in FR2 (h2A10.v2.5);
(vii) 49S in FR2 (h2A10.v2.6);
(viii) 73N in FR3 (h2A10.v2.7);
(ix) 76N in FR3 (h2A10.v2.8);
(x) 78L in FR3 (h2A10.v2.9); and
(xi) 49S in FR2, 76N, 78L in FR3 (h2A10.v3 and h2A10.v4).

Embodiment I-17. The antibody of Embodiment I-13 or Embodiment I-15 comprising a VL, wherein the VL retains leucine (L) at position 4 in framework I and leucine (L) at position 47 in framework II.

Embodiment I-18. The antibody of Embodiment I-15 or Embodiment I-17, wherein the VH retains D at position 73 in framework III of VH from r2A10.

Embodiment I-19. The anti-TGFβ3 antibody of any one of Embodiment I-1 to Embodiment I-12, wherein the antibody comprises a CDR-H2 having the amino acid sequence of SEQ ID NO: 5.

Embodiment I-20. The anti-TGFβ3 antibody of any one of Embodiment I-1 to Embodiment I-12, wherein the anti-TGFβ3 antibody comprises a CDR-H2 having the amino acid sequence of SEQ ID NO: 159.

Embodiment I-21. The anti-TGFβ3 antibody of Embodiment I-20, wherein the antibody comprises a VH amino acid sequence having at least 95%, at least 96%, at least 97%, at least 98%, at least 99%, or 100% sequence identity to the amino acid sequence of SEQ ID NO: 52.

Embodiment I-22. The anti-TGFβ3 antibody of Embodiment I-20 or Embodiment I-21, wherein the antibody comprises the VH amino acid sequence of SEQ ID NO: 52.

Embodiment I-23. The anti-TGFβ3 antibody of any of Embodiment I-20 to Embodiment I-22, wherein the antibody comprises a complete H chain amino acid sequence having at least 95%, at least 96%, at least 97%, at least 98%, at least 99%, or 100% sequence identity to the amino acid sequence of SEQ ID NO: 74.

Embodiment I-24. The anti-TGFβ3 antibody of any of Embodiment I-20 to Embodiment I-23, wherein the antibody comprises the complete H chain amino acid sequence of SEQ ID NO: 74.

Embodiment I-25. The anti-TGFβ3 antibody of any of Embodiment I-20 to Embodiment I-24, wherein the antibody comprises a VL amino acid sequence having at least 95%, at least 96%, at least 97%, at least 98%, at least 99%, or 100% sequence identity to an amino acid sequence selected from the group consisting of SEQ ID NOs: 22, 36, and 38-41.

Embodiment I-26. The anti-TGFβ3 antibody of any of Embodiment I-20 to Embodiment I-25, wherein the antibody comprises a VL amino acid sequence selected from the group consisting of SEQ ID NOs: 22, 36, and 38-41.

Embodiment I-27. The anti-TGFβ3 antibody of any of Embodiment I-20 to Embodiment I-26, wherein the antibody comprises a complete L chain amino acid sequence having at least 95%, at least 96%, at least 97%, at least 98%, at least 99%, or 100% sequence identity to an amino acid sequence selected from the group consisting of SEQ ID NOs: 28, 58, and 60-63.

Embodiment I-28. The anti-TGFβ3 antibody of any of Embodiment I-20 to Embodiment I-27, wherein the antibody comprises a complete L chain amino acid sequence selected from the group consisting of SEQ ID NOs: 28, 58, and 60-63.

Embodiment I-29. The anti-TGFβ3 antibody of Embodiment I-20, wherein the antibody comprises a VH/VL pair comprising the amino acid sequences (respectively) of SEQ ID NOs: 52/36.

Embodiment I-30. The anti-TGFβ3 antibody of Embodiment I-20, wherein the antibody comprises a complete H/L chain pair, the H/L chain pair comprising the amino acid sequences (respectively) of SEQ ID NOs: 74/58.

Embodiment I-31. The anti-TGFβ3 antibody of any one of Embodiment I-1 to Embodiment I-12, wherein the antibody comprises a CDR-H2 having the amino acid sequence of SEQ ID NO: 34.

Embodiment I-32. The anti-TGFβ3 antibody of Embodiment I-31, wherein the antibody comprises a VH amino acid sequence having at least 95%, at least 96%, at least 97%, at least 98%, at least 99%, or 100% sequence identity to the amino acid sequence of SEQ ID NO: 55.

Embodiment I-33. The anti-TGFβ3 antibody of Embodiment I-31 or Embodiment I-32, wherein the antibody comprises the VH amino acid sequence of SEQ ID NO: 55.

Embodiment I-34. The anti-TGFβ3 antibody of any of Embodiment I-31 to Embodiment I-33, wherein the antibody comprises a complete H chain amino acid sequence having at least 95%, at least 96%, at least 97%, at least 98%, at least 99%, or 100% sequence identity to the amino acid sequence of SEQ ID NO: 77.

Embodiment I-35. The anti-TGFβ3 antibody of any of Embodiment I-31 to Embodiment I-34, wherein the antibody comprises the complete H chain amino acid sequence of SEQ ID NO: 77.

Embodiment I-36. The anti-TGFβ3 antibody of any of Embodiment I-31 to Embodiment I-35 wherein the antibody comprises a VL amino acid sequence having at least 95%, at least 96%, at least 97%, at least 98%, at least 99%, or 100% sequence identity to the amino acid sequence of SEQ ID NO: 54.

Embodiment I-37. The anti-TGFβ3 antibody of any of Embodiment I-31 to Embodiment I-37, wherein the antibody comprises the VL amino acid sequence of SEQ ID NO: 54.

Embodiment I-38. The anti-TGFβ3 antibody of any of Embodiment I-31 to Embodiment I-37, wherein the antibody comprises a complete L chain amino acid sequence having at least 95%, at least 96%, at least 97%, at least 98%, at least 99%, or 100% sequence identity to the amino acid sequence of SEQ ID NO: 76.

Embodiment I-39. The anti-TGFβ3 antibody of any one of Embodiment I-31 to Embodiment I-38, wherein the antibody comprises the complete L chain amino acid sequence of SEQ ID NO: 76.

Embodiment I-40. The anti-TGFβ3 antibody of Embodiment I-31, wherein the antibody comprises a VH/VL pair, the VH/VL pair comprising the amino acid sequences (respectively) of SEQ ID NOs: 55/54.

Embodiment I-41. The anti-TGFβ3 antibody of Embodiment I-31, wherein the antibody comprises a complete H/L chain pair, the H/L chain pair comprising the amino acid sequences (respectively) of SEQ ID NOs: 77/76.

Embodiment I-42. The anti-TGFβ3 antibody of any one of Embodiment I-1 to Embodiment I-12, wherein the antibody comprises a CDR-H2 having the amino acid sequence of SEQ ID NO: 35.

Embodiment I-43. The anti-TGFβ3 antibody of Embodiment I-42, wherein the antibody comprises a VH amino acid sequence having at least 95%, at least 96%, at least 97%, at least 98%, at least 99%, or 100% sequence identity to the amino acid sequence of SEQ ID NO: 57.

Embodiment I-44. The anti-TGFβ3 antibody of Embodiment I-42 or Embodiment I-43, wherein the antibody comprises the VH amino acid sequence of SEQ ID NO: 57.

Embodiment I-45. The anti-TGFβ3 antibody of any of Embodiment I-42 to Embodiment I-44, wherein the antibody comprises a complete H chain amino acid sequence having at least 95%, at least 96%, at least 97%, at least 98%, at least 99%, or 100% sequence identity to the amino acid sequence of SEQ ID NO: 79.

Embodiment I-46. The anti-TGFβ3 antibody of any of Embodiment I-42 to Embodiment I-45, wherein the antibody comprises a complete H chain amino acid sequence of SEQ ID NO: 79.

Embodiment I-47. The anti-TGFβ3 antibody of any of Embodiment I-42 to Embodiment I-46, wherein the antibody comprises a VL amino acid sequence having at least 95%, at least 96%, at least 97%, at least 98%, at least 99%, or 100% sequence identity to the amino acid sequence of SEQ ID NO: 56.

Embodiment I-48. The anti-TGFβ3 antibody of any one of Embodiment I-42 to Embodiment I-47, wherein the antibody comprises the VL amino acid sequence of SEQ ID NO: 56.

Embodiment I-49. The anti-TGFβ3 antibody of any of Embodiment I-42 to Embodiment I-48, wherein the antibody comprises a complete L chain amino acid sequence having at least 95%, at least 96%, at least 97%, at least 98%, at least 99%, or 100% sequence identity to the amino acid sequence of SEQ ID NO: 76 Embodiment I-50. The anti-TGFβ3 antibody of any of Embodiment I-42 to Embodiment I-49, wherein the antibody comprises the complete L chain amino acid sequence of SEQ ID NOs: 76.

Embodiment I-51. The anti-TGFβ3 antibody of Embodiment I-42, wherein the antibody comprises a VH/VL pair comprising the amino acid sequences (respectively) of SEQ ID NOs: 57/56.

Embodiment I-52. The anti-TGFβ3 antibody of Embodiment I-42, wherein the antibody comprises a complete H/L chain pair, the H/L chain pair comprising the amino acid sequences (respectively) of SEQ ID NOs: 79/78.

Embodiment I-53. An anti-TGFβ3 antibody comprising:
(a) (i) heavy chain CDRs comprising CDR-H1, -H2 and -H3, wherein CDR-H1 has the amino acid sequence of SEQ ID NO: 4, CDR-H2 has the amino acid sequence of one of SEQ ID NO: 35, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9;
(b) a VH/VL pair, the VH of the VH/VL pair comprising the amino acid sequence of SEQ ID NO: 57, and the VL of the VH/VL pair comprising the amino acid sequence of SEQ ID NO: 56; or
(c) a complete H/L chain pair, the H chain of the H/L chain pair comprising the amino acid sequence of SEQ ID NO: 79 and the L chain of the H/L chain pair comprising the amino acid sequence of SEQ ID NO: 78.

Embodiment 90. An antibody of any one of Embodiments 1 to 89, or Embodiments I-1 to I-53, which is a monoclonal antibody.

Embodiment 91. An antibody of any one of Embodiments 1 to 89, or Embodiments I-1 to I-53, which is a human, humanized, or chimeric antibody.

Embodiment 92. An antibody of any one of Embodiments 1 to 89, or Embodiments I-1 to I-53, which is an antibody fragment.

Embodiment 93. An antibody of any one of Embodiments 1 to 89, or Embodiments I-1 to I-53, comprising a human Fc region that is an IgG1 or IgG4 isotype.

Embodiment 94. The antibody of Embodiment 93, comprising a human Fc region that is an IgG1 isotype.

Embodiment 95. The antibody of Embodiment 93 or 94, wherein the Fc region comprises a modification to remove the glycosylation site at amino acid residue position N297 (EU numbering as in Kabat).

Embodiment 96. The antibody of Embodiment 95, wherein the modification is a mutation selected from N297G or N297A.

Embodiment 97. The antibody of Embodiment 96, wherein the modification is the mutation N297G.

Embodiment 98. The antibody of any one of Embodiments 93 to 97, wherein the Fc region is modified to remove effector function.

Embodiment 99. An antibody of any one of Embodiments 1 to 98, or Embodiments I-1 to I-53, wherein the antibody has a Cmax of about 230-260 g/ml and/or a half life (t i) of about 15 to 16 days.

Embodiment 100. Isolated nucleic acid encoding the antibody of any one of Embodiments 1 to 99, or Embodiments I-1 to I-53.

Embodiment 101. A host cell comprising the nucleic acid of Embodiment 100.

Embodiment 102. A method of producing an antibody comprising culturing the host cell of Embodiment 101 so that the antibody is produced.

Embodiment 103. The method of Embodiment 102, further comprising recovering the antibody from the host cell.

Embodiment 104. An antibody produced by the method of Embodiment 102 or Embodiment 103.

Embodiment 105. An immunoconjugate comprising the antibody of Embodiment 1 and a cytotoxic agent.

Embodiment I-105. An immunoconjugate comprising the antibody of any one of Embodiments 1 to 89, or Embodiments I-1 to I-53, and a cytotoxic agent.

Embodiment 106. A pharmaceutical formulation comprising the antibody of any one of Embodiments 1 to 99, or Embodiments I-1 to I-53, or the immunoconjugate of Embodiment 105, or the immunoconjugate of Embodiment I-105, and a pharmaceutically acceptable carrier.

Embodiment 107. A pharmaceutical formulation comprising the antibody of Embodiment 106, further comprising an additional therapeutic agent.

Embodiment 108. The pharmaceutical formulation of Embodiment 107, wherein the additional therapeutic agent is selected from the group consisting of pirfenidone, nintedanib, mycophenylate mofetil, an IL-6 inhibitor (e.g., tocilizumab), an anti-CTFG antibody (e.g., FG-3019), an autotaxin inhibitor, a JAK inhibitor, an IL-11 inhibitor, and PTX2.

Embodiment 109. The antibody of any one of Embodiments 1 to 99, or Embodiments I-1 to I-53, for use as a medicament.

Embodiment 110. The antibody of any one of Embodiments 1 to 99, or Embodiments I-1 to I-53, for use in treating a TGFβ-related disorder.

Embodiment 111. The antibody of Embodiment 1 and the antibody of Embodiment 70, for use in combination to treat a TGFβ-related disorder.

Embodiment 112. The antibodies of Embodiment 110 or 111, wherein the TGFβ-related disorder is fibrosis.

Embodiment 113. The antibodies of Embodiment 112, wherein the fibrosis is a fibrotic condition of the lung, liver, heart, kidney, pancreas, eye, and/or skin.

Embodiment 114. The antibody of any one of Embodiments 1 to 99, or Embodiments I-1 to I-53, for use in the manufacture of a medicament for inhibiting TGFBR-dependent SMAD signaling, for inhibiting the assembly of TGFβ-TGFBR signaling complexes, for inhibiting TGFβ signaling through the TGFBR1/R2 complex, for inhibiting TGFβ signaling through the TGFBR2/ALK1 complex facilitated by endoglin, and/or for inhibiting new collagen synthesis.

Embodiment 115. Use of an antibody according to any one of Embodiments 1 to 99, or Embodiments I-1 to I-53, in the manufacture of a medicament for treating a TGFβ-related disorder in a subject.

Embodiment 116. The use of Embodiment 115, wherein the TGFβ-related disorder is fibrosis, wherein, optionally, the fibrosis is a fibrotic condition of the lung, liver, heart, kidney, pancreas, eye, and/or skin.

Embodiment 117. Use of an antibody of any one of Embodiments 1 to 99, or Embodiments I-1 to I-53, in the manufacture of a medicament for inhibiting TGFBR-dependent SMAD signaling, for inhibiting the assembly of TGFβ-TGFBR signaling complexes, for inhibiting TGFβ signaling through the TGFBR1/R2 complex, for inhibiting TGFβ signaling through the TGFBR2/ALK1 complex facilitated by endoglin, and/or for inhibiting new collagen synthesis.

Embodiment 118. A method of treating a subject having a TGFβ-related disorder, the method comprising administering to a subject in need thereof an effective amount of the antibody of any one of Embodiments 1 to 99, or Embodiments I-1 to I-53, or administering to the subject the pharmaceutical formulation of Embodiment 106.

Embodiment 119. A method for inhibiting TGFBR-dependent SMAD signaling, inhibiting the assembly of TGFβ-TGFBR signaling complexes, inhibiting TGFβ signaling through the TGFBR1/R2 complex, inhibiting TGFβ signaling through the TGFBR2/ALK1 complex facilitated by endoglin, and/or for inhibiting new collagen synthesis in a subject, comprising administering to a subject in need thereof an effective amount of the antibody of any one of Embodiments 1 to 99, or Embodiments I-1 to I-53, to inhibit TGFBR-dependent SMAD signaling, inhibiting the assembly of TGFβ-TGFBR signaling complexes, inhibiting TGFβ signaling through the TGFBR1/R2 complex, inhibiting TGFβ signaling through the TGFBR2/ALK1 complex facilitated by endoglin and/or for inhibiting new collagen synthesis in the subject.

Embodiment 120. The method of Embodiment 118 or 119, further comprising administering an additional therapeutic agent to the subject.

Embodiment 121. The method of Embodiment 120, wherein the additional therapeutic agent is selected from the group consisting of pirfenidone, nintedanib, mycophenylate mofetil, an IL-6 inhibitor (e.g., tocilizumab, sarilumab), an anti-CTFG antibody (e.g., FG-3019), an autotaxin inhibitor, and PTX2.

Embodiment 122. The method of any one of Embodiments 118 to 121, comprising administering the subject an effective amount of an anti-TGFβ3 antibody according to claim 1 and an effective amount of an anti-TGFβ2 antibody according to Embodiment 70.

Embodiment 123. The method of any one of Embodiments 118 to 122, wherein the subject has a TGFβ-related disorder that is fibrosis.

Embodiment 124. The method of Embodiment 123, wherein the fibrosis is a lung fibrosis selected from the group consisting of idiopathic pulmonary fibrosis (IPF), idiopathic pulmonary upper lobe fibrosis (Amitani disease), familial pulmonary fibrosis, pulmonary fibrosis (e.g., pulmonary fibrosis secondary to systemic inflammatory diseases such as, rheumatoid arthritis, scleroderma, lupus, cryptogenic fibrosing alveolitis, chronic obstructive pulmonary disease (COPD) or chronic asthma), cystic fibrosis, non-specific interstitial pneumonia (NSIP), cryptogenic organizing pneumonia (COP), progressive massive fibrosis, scleroderma/systemic sclerosis (SSc, including limited cutaneous (IcSSc) and diffuse cutaneous (dcSSc) forms and SSc-associated interstitial lung disease (SSc-ILD)), bronchiolitis obliterans-organizing pneumonia, connective tissue disease-associated ILD (CT-ILD), hypersensitivity pneumonitis, pulmonary hypertension, pulmonary tuberculosis, silicosis, asbestosis, acute lung injury, and acute respiratory distress (ARD, including bacterial pneumonia induced, trauma-induced, and viral pneumonia-induced, ventilator-induced, non-pulmonary sepsis induced).

Embodiment 125. The method of Embodiment 123, wherein the fibrosis is a fibrotic condition of the liver selected from the group consisting of liver cirrhosis, congenital hepatic fibrosis, obesity, fatty liver, alcohol induced liver fibrosis, non-alcoholic steatohepatitis (NASH), biliary duct injury, primary biliary cirrhosis (PBC), primary sclerosing cholangitis (PSC), infection- or viral-induced liver fibrosis (e.g., chronic hepatitis B and C virus infections), cystic fibrosis, autoimmune hepatitis, necrotizing hepatitis, primary sclerosing cholangitis, hemochromatosis, disorders of the biliary tree, and hepatic dysfunction attributable to infections.

Embodiment 126. The method of Embodiment 123, wherein the fibrosis is a fibrotic condition of the heart and/or pericardium selected from the group consisting of endomyocardial fibrosis, cardiac allograft vasculopathy (CAV), myocardial infarction, atrial fibrosis, congestive heart failure, arteriosclerosis, atherosclerosis, vascular stenosis, myocarditis, congestive cardiomyopathy, coronary infarcts, varicose veins, coronary artery stenosis and other post-ischemic conditions, and idiopathic retroperitoneal fibrosis.

Embodiment 127. The method of Embodiment 123, wherein the fibrosis is a fibrotic condition of the kidney selected from the group consisting of glomerulonephritis (including membranoproliferative, diffuse proliferative, rapidly progressive or sclerosing, post-infectious and chronic forms), diabetic glomerulosclerosis, focal segmental glomerulosclerosis, IgA nephropathy, diabetic nephropathy, ischemic nephropathy, tubulointerstitial kidney fibrosis, HIV-associated nephropathy, membrane nephropathy, glomerulonephritis secondary to systemic inflammatory diseases such as lupus, scleroderma and diabetes glomerulonephritis, idiopathic membranoproliferative glomerular nephritis, mesangial proliferative glomerulonephritis, crescentic glomerulonephritis, amyloidosis (which affects the kidney among other tissues), autoimmune nephritis, renal tubulointerstitial fibrosis, renal arteriosclerosis, Alport's syndrome, nephrotic syndrome, chronic renal failure, chronic kidney disease, periglomerular fibrosis/atubular glomeruli, combined apical emphysema and basal fibrosis syndrome (emphysema/fibrosis syndrome), glomerular hypertension, nephrogenic fibrosing dermatopathy, polycystic kidney disease, Fabry's disease, and renal hypertension.

Embodiment 128. The method of Embodiment 123, wherein the fibrosis is a fibrotic condition of the pancreas selected from the group consisting of stromal remodeling pancreatitis and stromal fibrosis.

Embodiment 129. The method of Embodiment 123, wherein the fibrosis is a fibrotic condition of the gastrointestinal tract selected from the group consisting of Crohn's disease, ulcerative colitis, collagenous colitis, colorectal fibrosis, villous atrophy, crypt hyperplasia, polyp formation, healing gastric ulcer, and microscopic colitis.

Embodiment 130. The method of Embodiment 123, wherein the fibrosis is a fibrotic condition of the eye selected from the group consisting of ocular fibrosis, ophthalmic fibrosis, proliferative vitreoretinopathy, vitreoretinopathy of any etiology, fibrosis associated with retinal dysfunction, fibrosis associated with wet or dry macular degeneration, scarring in the cornea and conjunctiva, fibrosis in the corneal endothelium, anterior subcapsular cataract and posterior capsule opacification, anterior segment fibrotic diseases of the eye, fibrosis of the corneal stroma (e.g., associated with corneal opacification), fibrosis of the trabecular network (e.g., associated with glaucoma), posterior segment fibrotic diseases of the eye, fibrovascular scarring (e.g., in retinal or choroidal vasculature of the eye), retinal fibrosis, epiretinal fibrosis, retinal gliosis, subretinal fibrosis (e.g., associated with age related macular degeneration), tractional retinal detachment in association with contraction of the tissue in diabetic retinopathy, congenital orbital fibrosis, lacrimal gland fibrosis, corneal subepithelial fibrosis, and Grave's ophthalmopathy.

Embodiment 131. The method of Embodiment 123, wherein the fibrosis is selected from fibrosis resulting from spinal cord injury/fibrosis or central nervous system fibrosis such as fibrosis after a stroke, Duchenne muscular dystrophy, fibrosis associated with neurodegenerative disorder such as Alzheimer's disease or multiple sclerosis, vascular restenosis, uterine fibrosis, endometriosis, ovarian fibroids, Peyronie's disease, polycystic ovarian syndrome, disease related pulmonary apical fibrosis in ankylosing spondylitis, scarring, and fibrosis incident to microbial infections (e.g., bacterial, viral, parasitic, fungal).

Embodiment 132. The method of Embodiment 123, wherein the fibrosis is SSc.

Embodiment 133. The method of Embodiment 123, wherein the fibrosis is IPF.

Embodiment 134. The method of Embodiment 123, wherein the fibrosis is chronic obstructive pulmonary disease (COPD).

Embodiment 135. The method of Embodiment 123, wherein the fibrosis is progressive-fibrosing interstitial lung disease (PF-ILD).

Embodiment 136. The method of Embodiment 135, wherein the PF-ILD is a disease or condition selected from the group consisting of non-specific interstitial pneumonia (NSIP), cryptogenic organizing pneumonia (COP), progressive massive fibrosis, a complication of coal worker's pneumoconiosis, scleroderma/systemic sclerosis, bronchiolitis obliterans-organizing pneumonia; connective tissue disease-associated ILD (CT-ILD), and hypersensitivity pneumonitis.

Embodiment 137. The method of Embodiment 123, wherein the fibrosis is liver cirrhosis or chronic hepatic fibrosis.

Embodiment 138. The method of Embodiment 123, wherein the fibrosis is GI tract fibrosis, e.g., intestinal fibrosis, optionally, selected from the group consisting of fibrosis associated with Crohn's disease, ulcerative colitis, collagenous colitis, colorectal fibrosis, villous atrophy, crypt hyperplasia, polyp formation, healing gastric ulcer, and microscopic colitis.

Embodiment 139. The method of Embodiment 123, wherein the fibrosis is a fibrotic condition of the eye, fibrosis resulting from spinal cord injury, fibrosis or central nervous system fibrosis, or fibrosis associated with a neurodegenerative disorder.

Embodiment 140. The method of any one of Embodiments 118 to 139, wherein the antibody is an anti-TGFβ2 antibody.

Embodiment 141. The method of Embodiment 140, wherein the anti-TGFβ2 antibody comprises:
(a) heavy chain CDRs comprising CDR-H1, -H2 and -H3, wherein CDR-H1 has the amino acid sequence of SEQ ID NO: 16, CDR-H2 has the amino acid sequence of SEQ ID NO: 17, and CDR-H3 has the amino acid sequence of SEQ ID NO: 18; and (b) light chain CDRs comprising CDR-L1, -L2 and -L3, wherein CDR-L1 has the amino acid sequence of SEQ ID NO: 19; CDR-L2 has the amino acid sequence of SEQ ID NO: 20, and CDR-L3 has the amino acid sequence of SEQ ID NO: 21.

Embodiment 142. The method of any one of Embodiments 118 to 139, wherein the antibody is an anti-TGFβ3 antibody.

Embodiment 143. The method of Embodiment 142, wherein the anti-TGFβ3 antibody comprises:
(a) (i) heavy chain CDRs comprising CDR-H1, -H2 and -H3, wherein CDR-H1 has the amino acid sequence of SEQ ID NO: 4, CDR-H2 has the amino acid sequence of one of SEQ ID NO: 35, and CDR-H3 has the amino acid sequence of SEQ ID NO: 6; and (ii) light chain CDRs comprising CDR-L1, -L2 and -L3, wherein CDR-L1 has the amino acid sequence of SEQ ID NO: 7; CDR-L2 has the amino acid sequence of SEQ ID NO: 8, and CDR-L3 has the amino acid sequence of SEQ ID NO: 9;
(b) a VH/VL pair, the VH of the VH/VL pair comprising the amino acid sequence of SEQ ID NO: 57, and the VL of the VH/VL pair comprising the amino acid sequence of SEQ ID NO: 56; or
(c) a complete H/L chain pair, the H chain of the H/L chain pair comprising the amino acid sequence of SEQ ID NO: 79 and the L chain of the H/L chain pair comprising the amino acid sequence of SEQ ID NO: 78.

Embodiment 144. The method of any one of Embodiments 118 to 139, wherein the antibody is an anti-TGFβ2/3 antibody.

Embodiment 145. The method of Embodiment 144, wherein the anti-TGFβ2/3 antibody comprises: (i) heavy chain CDRs comprising CDR-H1, -H2 and -H3, wherein CDR-H1 has the amino acid sequence of SEQ ID NO: 10, CDR-H2 has the amino acid sequence of SEQ ID NO: 11, and CDR-H3 has the amino acid sequence of SEQ ID NO: 12; and (ii) light chain CDRs comprising CDR-L1, -L2 and -L3, wherein CDR-L1 has the amino acid sequence of SEQ ID NO: 13; CDR-L2 has the amino acid sequence of SEQ ID NO: 14, and CDR-L3 has the amino acid sequence of SEQ ID NO: 15; Embodiment 146. A method of diagnosing a subject as having SSc, the method comprising detecting the expression levels of the genes in an 18-gene signature set consisting of PRSS23, PXDN, COL8A1, COL6A3, SERPINE2, TNC, COMP, THBS1, COL11A1, COL1A1, COL5A2, COL1A2, COL4A1, COL4A2, SFRP4, ALPK2, COL5A1, and TAGLN, and diagnosing the subject with SSc if the levels of the genes are determined to be elevated relative to the gene levels in a healthy control set or reference gene signature.

Embodiment 147. The method of Embodiment 146, wherein a gene level is elevated if the expression increase relative to the healthy control set or reference gene signature is statistically significant, optionally, at least two-fold increased, or at least three-fold increased, or at least four-fold increased, relative to the healthy control set or reference gene signature.

Embodiment 148. A method of monitoring response to treatment of a subject with an anti-TGFβ2 antibody and/or an anti-TGFβ3 antibody, the method comprising determining the expression level of one or more of the TGFβ-inducible genes selected from the group consisting of serpine1, col1a1 col1a2, and col3a1 in a sample from the subject, wherein the subject has received one or more administrations of the anti-TGFβ2 antibody and/or the anti-TGFβ3 antibody.

Embodiment 149. The method of Embodiment 148, wherein the subject is determined to be responding to treatment with the anti-TGFβ2 antibody and/or anti-TGFβ3 antibody, if the expression level of the one or more TGFβ-inducible genes is significantly reduced compared to pre-treatment levels of the one or more TGFβ-inducible genes, wherein optionally the method further comprises administering an additional administration of the anti-TGFβ2 antibody and/or the anti-TGFβ3 antibody if the expression level of the one or more TGFβ-inducible genes is determined to be significantly reduced.

Embodiment 150. The method of Embodiment 148 or 149, wherein the subject is administered the anti-TGFβ2 antibody as a monotherapy.

Embodiment 151. The method of Embodiment 148 or 149, wherein the subject is administered the anti-TGFβ3 antibody as a monotherapy.

Embodiment 152. The method of Embodiment 148, wherein the expression level of the one or more TGFβ-inducible genes is determined by qPCR or microarray analysis.

Embodiment 153. The method or use of any one of Embodiments 100 to 152, wherein the subject is a human.

Embodiment 154. A kit comprising an antibody of any one of Embodiments 1 to 99, or Embodiments I-1 to I-53.

Embodiment I-155. A method for treating a TGFβ-related disorder in a subject in need thereof, the method comprising:
determining that a sample from the subject comprises an elevated level of one or more biomarkers; and
administering to the subject an effective amount of an isoform-selective anti-TGFβ antibody.

Embodiment I-156. A method for selecting a therapy for a subject with an autoimmune or inflammatory disease, comprising:
determining the level of one or more biomarkers of the subject; and selecting a medicament based on the level of the one or more biomarkers, wherein the medicament selected is an isoform-selective anti-TGFβ antibody if the level of one or more of the biomarkers is elevated.

Embodiment I-157. A method for treating a TGFβ-related disorder in a subject in need thereof, comprising administering to the subject an effective amount of an isoform-selective anti-TGFβ antibody, wherein prior to beginning administration, the subject has an elevated level of one or more biomarkers.

Embodiment I-158. The method of any one of Embodiments I-155 to I-157, wherein the one or more biomarkers are selected from the group consisting of TGFβ2, TGFβ3, periostin (POSTN), and cartilage oligomeric matrix protein (COMP).

Embodiment I-159. The method of any one of Embodiments I-155 to I-157, wherein the one or more biomarkers are selected from the group consisting of PRSS23, PXDN, COL8A1, COL6A3, SERPINE2, TNC, COMP, THBS1, COL11A1, COL1A1, COL5A2, COL1A2, COL4A1, COL4A2, SFRP4, ALPK2, COL5A1, and TAGLN.

Embodiment I-160. The method of any one of Embodiments I-155 to I-157, wherein the one or more biomarkers are selected from the group consisting of SERPINE1, COL1A1, COL1A2, and COL3A1.

Embodiment I-161. The method of any one of Embodiments I-155 to I-160, wherein the level of one or more biomarkers are elevated in a sample from the subject, wherein the sample is a skin, lung, or blood sample.

Embodiment I-162. The method of Embodiments I-158, wherein the sample is a serum sample.

Embodiment I-160. The method of any one of Embodiments I-155 to I-159, wherein the isoform-selective anti-TGFβ antibody is an anti-TGFβ2, anti-TGFβ2/3, or anti-TGFβ3 antibody.

Embodiment I-161. The method of any one of Embodiments I-155 to I-160, wherein the elevated biomarkers are one or both of periostin and COMP.

Embodiment I-162. The method of any one of Embodiments I-155 to I-161, wherein the disorder is a fibrotic disorder.

Embodiment I-163. The method of Embodiment I-162, wherein the fibrotic disorder is lung fibrosis, a fibrotic condition of the liver, a fibrotic condition of the heart and/or pericardium, a fibrotic condition of the kidney, a fibrotic condition of the pancreas, a fibrotic condition of the gastrointestinal tract, a fibrotic condition of the eye, or a fibrosis resulting from spinal cord injury/fibrosis or central nervous system fibrosis.

Embodiment I-164. The method of Embodiment I-162, wherein the fibrotic disorder is selected from the group consisting of IPF, SSc, COPD, and. PF-ILD.

Embodiment I-165. The method of any one of Embodiments I-155 to I-164, wherein the elevated level is relative to a healthy individual or control group, and wherein the elevation is statistically significant, optionally, at least two-fold increased, or at least three-fold increased, or at least four-fold increased, relative to the healthy individual or control group.

Embodiment I-166. The method of any one of Embodiments I-155 to I-164, wherein the subject was not responsive to anti-IL-6 inhibitor therapy.

Embodiment I-167. The method of Embodiment I-166, wherein the anti-IL-6 therapy was tocilizumab.

Embodiment I-168. The method of any one of Embodiments I-155 to I-167, wherein the isoform-selective anti-TGFβ is an antibody of any one Embodiments 1 to 99, or Embodiments I-1 to I-53.

### IV. Examples

The following are examples of methods and compositions of the invention. It is understood that various other embodiments may be practiced, given the general description provided above.

### Example 1: Generation and Screening of Isoform-Selective TGFβ Antibodies

### Materials and Methods

### Generation of Rabbit Anti-Human TGFβ Monoclonal Antibodies

New Zealand white rabbits were immunized with human TGFβ2 and TGFβ3 isoforms (Peprotech) and single B-cell was isolated using a modified protocol related to published literature (Offner et al. PLoS ONE 9(2), 2014). Briefly, rabbit peripheral blood mononuclear cells (PBMCs) were isolated by density centrifugation of blood (1:1 dilution with PBS) collected from ear artery using Lympholyte®-M (Cedarlane Labs). After washing with PBS, PBMCs were resuspended in culture medium RPMI with supplement and transferred to 6-well plate to remove macrophages and monocytes through non-specific adhesion onto the plate. The non-adhesion cells were then collected for B cell enrichment by immunofluorescence staining with biotinylated anti-rabbit CD11b antibody, anti-rabbit T-lymphocyte antibody (AbD Serotec, BioRad) and anti-rabbit IgM (BD Bioscience) to further deplete rabbit myeloid cells, T cells and IgM B cells through MACS Column (Miltenyl Biotec) according to the manufacturer's instructions. The following workflow included using FITC-labeled goat anti-rabbit IgG antibody (Serotec, BioRad), APC-labeled human TGFβ1 (Innova Biosciences) and RPE-labeled human TGFβ2/3 (Innova Biosciences) to sort out rabbit IgG+ and human TGFβ2/3+-reactive B cells into single wells. The B-cell culture supernatants were subject to primary ELISA screening for human TGFβ2/3 isoforms binding, and B-cells were lysed and stored at −80 °C. In the molecular cloning, the light chain and heavy chain variable region of human TGFβ2/3 specific B cells were amplified by PCR and cloned into expression vectors as described in the published literature (Offner et al. PLoS ONE 9(2), 2014). Each recombinant rabbit monoclonal antibody was expressed in Expi293 cells (Thermo Fisher Scientific, Waltham, MA) and subsequently purified with protein A. Purified anti-human TGFβ2/3 antibodies were then subjected to functional characterization in blocking, affinity determination.

### Development of Rat Anti-Human TGFβ Hybridoma Antibodies

Sprague Dawley rats (Charles River, Hollister, CA) were immunized every two weeks with 100 g of human TGFβ2 or TGFβ3 proteins (Peprotech) mixed with RIBI adjuvant (Sigmal, St. Louis, MO) and injected at base of tail, or mixed with Incomplete Freund's Adjuvant (BD) and divided among multiple sites: I.P., S.C. at base of tail, s.c. at nape of neck, and s.c. in both hocks. Multiple lymph nodes were harvested three days after the last immunization. IgM negative B-cells from these rats were purified from lymphocytes using magnetic separation (Miltenyi Biotec, San Diego, CA) and were fused with Sp2ab mouse myeloma cells (Enzo Life Sciences, Farmingdale, NY) via electrofusion (Harvard Apparatus, Holliston, MA). Fused cells were incubated at 37 °C, 7% CO2, overnight in Medium C (StemCell Technologies, Vancouver, BC, Canada), before plating into 6-well plates containing Medium E (StemCell Technologies, Vancouver, BC, Canada) supplemented with HAT (Sigma-Aldrich, St. Louis, MO) and incubated at 37 °C, 7% CO2 for three days. TGFβ2 hybridomas were collected and sorted with anti-rat IgG (Sigma-Aldrich) and TGFβ3 hybridomas were sorted with anti-rat IgG (Sigma-Aldrich) and labeled human TGFβ3-PE by Lightning-Link R-PE Antibody Labeling kit (Novus, Centennial, CO).

### HEKBlue Cell-Based TGFβ Blocking Assay

HEK-Blue™ TGFβ cells (InvivoGen, San Diego, CA) stably expressing human TGFβRI, Smad3, Smad4 proteins and secreted alkaline phosphatase (SEAP) reporter gene under the control of the β-globin minimal promoter fused to three Smad3/4-binding elements (SBE) were used to evaluate the blocking potency of monoclonal antibodies generated from rats and rabbits immunized with the mature receptor-binding domains of human TGFβ1, TGFβ2 and TGFβ3. The antibodies were incubated with each TGFβ isoforms (PeproTech, Rocky Hill, NJ) at 25 °C for 1 h prior to the addition into 4.5×104 HEK-Blue™ TGF-β cells seeded in 96-well plate in 100 L of assay buffer (DMEM high glucose with 10% heat inactivated FBS, 2 mM L-glutamine and 0.5% Penicillin Streptomycin). The final concentration of antibody and TGFβ was 0.001-200 nM and 20 pM respectively. The plate was incubated at 37 °C and 5% CO₂ for 18-22 h. The SEAP level in the cell supernatant was determined by QUANTI-Blue™ assay according to the manufacturer's instructions (InvivoGen). Pan-TGFβmAb neutralizes TGFβ1, 2, and 3 at low cellular potencies (IC50) of 0.72, 2.2, and 0.026 nM, respectively.

### ELISA Screening

An enzyme-linked immunosorbent assay (ELISA) was used to screen the binding of candidate clones to TGFβ1, TGFβ2, and TGFβ3. First, each TGFβ isoform (Peprotech) was immobilized to the wells of Grenier Bio-One 384-well ELISA plates with coating buffer (50 mM carbonate, pH 9.6), at 1 μg/ml; 50 μL/well for overnight incubation at 4 °C. Then the coated ELISA plates were washed 3 times with ELISA diluent buffer 100 μl per well (PBS/0.5% Tween20). Next the ELISA plates were blocked with ELISA diluent buffer 100 μl/well for one hour. After blocking, the buffer was discarded and samples and controls were added from 96-well plates to 384-well plates (50 μl/well). Then the ELISA plates with sample in were incubated for 30 minutes at room temperature while the plates were shaking on the shaker. Next the plates were washed three times with wash buffer (PBS/0.05% TWEEN 20, 20X Stock Hyclone SH3A649-01) 100 ul/wee. Then diluted HRP conjugate (Peroxidase AffiniPure Goat Anti-Rat IgG (H+L) Jackson ImmunoResearch Laboratories, INC. 112-035-003 diluted 1:10000, 50 μl/well) was added to each well and the ELISA plates were incubated for 30 minutes at room temperature. After the incubation, the plates were washed 3 times with wash buffer (100 μl/well) and then added substrate (BIOFX TMB Substrate TMBW-1000-01, 50 ul/well). After 5 minutes incubation at room temperature for 5 minutes, the stop solution (BIOFX Stop reagent LBSP-1000-01, 50 μl/well) was added. Finally, the plates were read at 659 nm for absorbance.

### Results:

Monoclonal antibodies generated from rats and rabbits immunized with the mature receptor-binding domains of human TGFβ1, TGFβ2 and TGFβ3 were screened for binding via direct ELISA and surface plasmon resonance (SPR), and for their ability to inhibit mature receptor-binding domain induced TGFβR-dependent signaling in a reporter cell line (HEKBlue SMAD2/3 SEAP reporter), as described above. Out of 96 initial rabbit clones that exhibited binding to TGFβ2 and 85 clones that bound to TGFβ3, 48 clones were selected for further screening. For the rabbit antibodies, 1,995 IgG+ TGFβ2 hybridomas and 2,660 IgG+TGFβ3+ hybridomas were single-cell sorted using a FACSAria Fusion sorter (BD Biosciences, Franklin Lakes, NJ) into 96-well plates. Seven days after plating, supernatants were screened by ELISA against human TGFβ2 or TGFβ3 proteins. Supernatants from 28 TGFβ2 and 48 TGFβ3 hybridomas demonstrating binding to their respective antigens by ELISA were harvested and purified by protein G (GammaBind Plus, GE Healthcare, Pittsburgh, PA). Among the 28 TGFβ2+ clones, 4 showed functional blocking against TGFβ2, including 6F12. Among the 48 TGFβ3+ clones, 28 clones showed functional blocking against human TGFβ3, including 2A10.

Potent inhibitory antibodies (K_{D}<10 pM and cell-based IC₅₀<250 pM) were identified with selectivity for TGFβ2 (mAb 6F12 (rat)), TGFβ3 (mAbs 16C10 (rabbit) and 2A10 (rat)), and a dual-specificity antibody that bound to and inhibited the activity of both TGFβ2 and TGFβ3 (mAb 4A11 (rabbit)), but none of these antibodies bound to or inhibited TGFβ1 (see Table 4, below).

The relative potencies of these antibodies for selected TGFβ isoforms were comparable or superior to the activity of a “pan-TGFβ” antibody, 1D11 (Dasch, J R et al. J. Immunol. 142: 1536-41 (1989)) (see Table 4, below). Notably, the TGFβ2-specific antibody 6F12 was nearly 100-fold more potent than 1D11 in blocking TGFβ2-dependent reporter activity in cells (IC₅₀ of 40 pM vs. 3.6 nM).

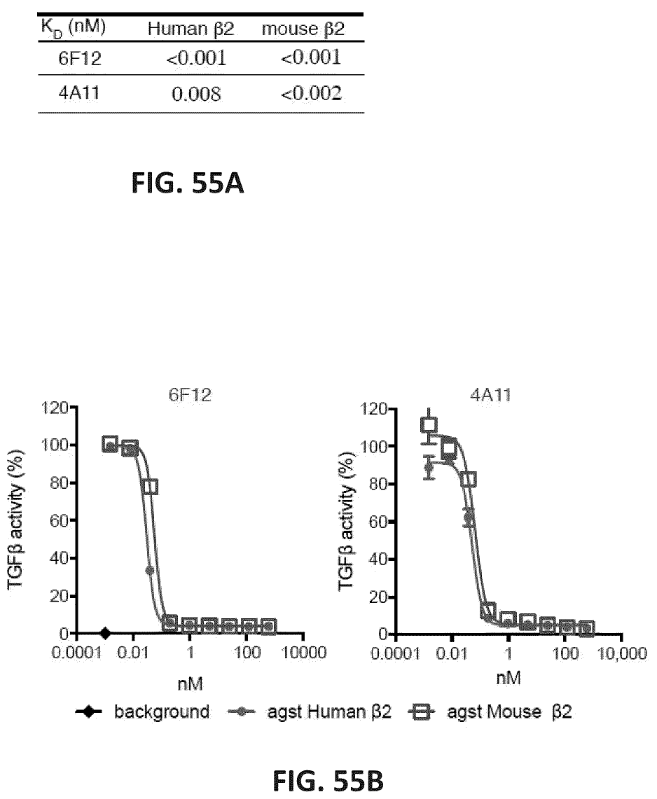

Further, there are three amino acid differences between human and mouse mature TGFβ2, and 6F12 and 4A11 bind to both peptides with similar affinities (FIG. 55A) and have similar inhibitory potencies against both (FIG. 55B).

TABLE 4
Potencies of Isoform-selective TGFβ Antibodies

| | | Affinity @ 25 °C (nM) | | | Blocking @ 37 °C (nM) | | |
|---|---|---|---|---|---|---|---|
| Function | Clone | TGFb1 | TGFb2 | TGFb3 | TGFb1 | TGFb2 | TGFb3 |
| TGFb1 blocker | 21D1 | 0.008 | >5 | >5 | 0.013 | >200 | >200 |
| | 20C8 | 0.001 | >25 | >25 | 0.13 | >70 | >70 |
| | 39A3 | 0.085 | 0.7 | >25 | 0.03 | >70 | >70 |
| | 40F8 | 0.003 | >25 | >25 | 0.02 | >70 | >70 |
| TGFβ1 IHC reagent | Rb3.22.C6 | 0.022 | >5 | >5 | 0.5 | 69.3 | >70 |
| | Rb3.23.B6 | 0.021 | >5 | >5 | 20.4 | 29.5 | >70 |
| | Rb3.32.F8 | >5 | >5 | >5 | 3.58 | >70 | >70 |
| TGFb2 blocker | 6F12 | >15 | <0.001 | >15 | >600 | 0.04 | >600 |
| | 36C5 | >25 | 0.003 | 4.9 | >200 | 0.002 | >200 |
| | 1C3 | 0.25 | <0.004 | >25 | >200 | 0.002 | >200 |
| | 9B10 | >25 | 0.003 | 0.21 | >200 | 0.018 | >200 |
| | 31E12 | >25 | 0.003 | 29 | >200 | 0.015 | >200 |
| | 19D3 | >25 | 0.005 | >25 | >200 | 0.015 | >200 |
| | 1G10 | 3.9 | 0.007 | >25 | >200 | 0.011 | >200 |
| TGFβ2 IHC reagent | R62.14.F11 | >5 | 0.004 | 0.521 | >67 | 0.05 | >67 |
| | Rb2.20.F10 | >5 | 0.007 | >5 | NA | | |
| TGFb3 IHC reagent | Rb1.9A8 | >15 | >15 | 0.014 | >200 | >200 | 3.0 |
| & TGFb3 blocker | Rb2.15A7 | >15 | >15 | 0.006 | >200 | >200 | 0.4 |
| | Rb2.16C10 | >15 | 0.19 | <0.003 | >200 | >200 | 0.1 |
| TGFb3 blocker | 2A10 | 1.6 | 0.046 | <0.002 | >600 | 193 | 0.02 |
| | Rb1.9A10 | 0.025 | 0.723 | 0.026 | >200 | >200 | 0.4 |
| | Rb1.25D11 | >15 | >15 | 0.012 | >200 | >200 | 50 |
| TGFb1&2 blocker | Rb1.22C7 | 0.036 | 0.006 | 6.1 | 3.64 | 0.04 | >200 |
| TGFb2&3 blocker | R61.34E4 | >15 | 0.002 | 0.004 | >200 | 0.37 | 1.8 |
| | Rb2.14D7 | >15 | 0.006 | >15 | >200 | 0.02 | 14 |
| | Rb2.13G11 | >15 | 0.143 | >15 | >200 | 0.42 | 28.3 |
| | Rb1.7H10 | >15 | <0.001 | 0.004 | >200 | 1.1 | 2.1 |
| | Rb2.15C2 | 1.9 | 0.23 | 0.41 | >200 | 2.83 | 4.2 |
| | Rb1.4A11 | >15 | 0.008 | 0.005 | >600 | 0.25 | 0.03 |
| | Rb2.11E6 | >15 | 0.11 | 0.019 | >200 | 5.5 | 0.3 |
| | 32F10 | 0.05 | 0.04 | 0.1 | >200 | 0.04 | 0.96 |
| PanTGFb blocker | 1D11 | 0.038 | 0.009 | 0.007 | 0.5 | 3.6 | 0.04 |
| | Rb1.16D9 | <0.001 | <0.001 | 0.084 | 0.01 | 0.01 | 4.86 |
| | Rb1.14E9 | 0.002 | <0.001 | 0.009 | 1.44 | 0.02 | 1.77 |
| | Rb2.19H8 | 0.005 | 0.003 | 0.007 | 11 | 0.02 | 0.12 |
| | 11A8 | 0.015 | 0.004 | 0.003 | 0.36 | 0.015 | 0.017 |

The sequences of the monoclonal antibodies are provided in Tables 5-7 below.

TABLE 5
CDR Sequences of anti-TGFβ isoform-selective monoclonal antibodies

| | CDR H1 | CDR H2 | CDR H3 | CDR L1 | CDR L2 | CDR L3 |
|---|---|---|---|---|---|---|
| 2A10 | SYGMS | DIVSKTYNYT | APGGSF | RASQSV | RASNLAS | QHSRESP |
| (anti- | (SEQ ID | TYYSDSVKD | DY | SISRFNL | (SEQ ID | WT |
| TGFβ3) | NO: 4) | (SEQ ID NO: 5) | (SEQ ID | MH | NO: 8) | (SEQ ID |
| | | | NO: 6) | (SEQ ID | | NO: 9) |
| | | | | NO: 7) | | |
| 4A11 | SYTVN | YISYGGSAYY | HMQVG | QSSQSV | GASTLTS | AGGYSGS |
| (anti- | (SEQ ID | ASWANG | GAPTGS | YNNNYL | (SEQ ID | SDKYA |
| TGFβ2/ | NO: 10) | (SEQ ID NO: | MAAFD | S (SEQ ID | NO: 14) | (SEQ ID |
| 3) | | 11) | P (SEQ | NO: 13) | | NO: 15) |
| | | | ID NO: | | | |
| | | | 12) | | | |
| 6F12 | TYNVH | LIWNTGGTRY | DPVPN | LASEDIY | DARSLQD | QQHHAYP |
| (anti- | (SEQ ID | NSALKS (SEQ | KWHFD | SNLA | (SEQ ID | FT (SEQ ID |
| TGFβ2) | NO: 16) | ID NO: 17) | F (SEQ | (SEQ ID | NO: 20) | NO: 21) |
| | | | ID NO: | NO: 19) | | |
| | | | 18) | | | |

TABLE 6
Heavy and Light Chain Variable Region Sequences of Isoform-selective
anti-TGFβ Monoclonal Antibodies

| mAb | VH | VL |
|---|---|---|
| 2A10 | EVQLVESGGGLVQPKGSLKLSCAASGF | DIVLTQSPALAVSLGQRATISCRASQSV |
| | DFNSYGMSWVRQAPGKGLDLVADIVS | SISRFNLMHWYQHKPGQQPKLLIYRAS |
| | KTYNYTTYYSDSVKDRFTISRDDSQSM | NLASGIPARFSGSGSGTDFTLTINPVQAD |
| | VYLQMDNLKTEDTALYYCTVAPGGSF | DLATYYCQHSRESPWTFGGGTKLEIK |
| | DYWGQGVMVTVSS (SEQ ID NO: 23) | (SEQ ID NO: 22) |
| 6F12 | QVQLKESGPGLVQPSQTLSLTCTVSGFS | DIQMTQSPASLSASLGETVTIECLASEDI |
| | LTTYNVHWVRQPPGKGLEWMGLIWNT | YSNLAWYQQKPGKSPQLLIYDARSLQD |
| | GGTRYNSALKSRLSISKDTSKSQVFLRM | GVPSRFSGSESGPQYSLEINSLQSEDAVT |
| | NSLQTEDTATYYCARDPVPNKWHFDF | YFCQQHHAYPFTFGSGTKLEIK (SEQ ID |
| | WGPGTMVTVSS (SEQ ID NO: 25) | NO: 24) |
| 4A11 | QSLEESGGRLVTPGTPLTLTCTVSGFSLS | AAVLTQTPSPVSAAVGGTVSISCQSSQS |
| | SYTVNWVRQAPGKGLEWIGYISYGGSA | VYNNNYLSWFQQKPGQPPKLLIYGAST |
| | YYASWANGRFTISKTSATVDLKITSPTT | LTSGVPSRFKGSGSGTQFTLTISDVQCD |
| | EDTATYFCARHMQVGGAPTGSMAAFD | DAATYYCAGGYSGSSDKYAFGGGTEV |
| | PWGPGTLVTVSS (SEQ ID NO: 27) | WK (SEQ ID NO: 26) |

TABLE 7
Complete Heavy and Light Chain Sequences of Isoform-selective anti-TGFβ
monoclonal antibodies

| mAb | Complete H Chain | Complete L Chain |
|---|---|---|
| 2A10 | EVQLVESGGGLVQPKGSLKLSCAASGFDF | DIVLTQSPALAVSLGQRATI |
| | NSYGMSWVRQAPGKGLDLVADIVSKTYN | SCRASQSVSISRFNLMHWY |
| | YTTYYSDSVKDRFTISRDDSQSMVYLQMD | QHKPGQQPKLLIYRASNLA |
| | NLKTEDTALYYCTVAPGGSFDYWGQGVM | SGIPARFSGSGSGTDFTLTIN |
| | VTVSSAKTTAPSVYPLAPVCGDTTGSSVTL | PVQADDLATYYCQHSRESP |
| | GCLVKGYFPEPVTLTWNSGSLSSGVHTFPA | WTFGGGTKLEIKRADAAPT |
| | VLQSDLYTLSSSVTVTSSTWPSQSITCNVA | VSIFPPSSEQLTSGGASVVCF |
| | HPASSTKVDKKIEPRGPTIKPCPPCKCPAPN | LNNFYPKDINVKWKIDGSE |
| | AAGGPSVFIFPPKIKDVLMISLSPIVTCVVV | RQNGVLNSWTDQDSKDST |
| | DVSEDDPDVQISWFVNNVEVHTAQTQTHR | YSMSSTLTLTKDEYERHNS |
| | EDYNSTLRVVSALPIQHQDWMSGKEFKCK | YTCEATHKTSTSPIVKSFNR |
| | VNNKDLGAPIERTISKPKGSVRAPQVYVLP | NEC (SEQ ID NO: 28) |
| | PPEEEMTKKQVTLTCMVTDFMPEDIYVEW | |
| | TNNGKTELNYKNTEPVLDSDGSYFMYSKL | |
| | RVEKKNWVERNSYSCSVVHEGLHNHHTT | |
| | KSFSRTPGK (SEQ ID NO: 29) | |
| 6F12 | QVQLKESGPGLVQPSQTLSLTCTVSGFSLT | DIQMTQSPASLSASLGETVT |
| | TYNVHWVRQPPGKGLEWMGLIWNTGGTR | IECLASEDIYSNLAWYQQKP |
| | YNSALKSRLSISKDTSKSQVFLRMNSLQTE | GKSPQLLIYDARSLQDGVPS |
| | DTATYYCARDPVPNKWHFDFWGPGTMVT | RFSGSESGPQYSLEINSLQSE |
| | VSSAKTTAPSVYPLAPVCGDTTGSSVTLGC | DAVTYFCQQHHAYPFTFGS |
| | LVKGYFPEPVTLTWNSGSLSSGVHTFPAVL | GTKLEIKRADAAPTVSIFPPS |
| | QSDLYTLSSSVTVTSSTWPSQSITCNVAHP | SEQLTSGGASVVCFLNNFYP |
| | ASSTKVDKKIEPRGPTIKPCPPCKCPAPNAA | KDINVKWKIDGSERQNGVL |
| | GGPSVFIFPPKIKDVLMISLSPIVTCVVVDV | NSWTDQDSKDSTYSMSSTL |
| | SEDDPDVQISWFVNNVEVHTAQTQTHRED | TLTKDEYERHNSYTCEATH |
| | YNSTLRVVSALPIQHQDWMSGKEFKCKVN | KTSTSPIVKSFNRNEC (SEQ |
| | NKDLGAPIERTISKPKGSVRAPQVYVLPPPE | ID NO: 30) |
| | EEMTKKQVTLTCMVTDFMPEDIYVEWTN | |
| | NGKTELNYKNTEPVLDSDGSYFMYSKLRV | |
| | EKKNWVERNSYSCSVVHEGLHNHHTTKSF | |
| | SRTPGK (SEQ ID NO: 31) | |
| 4A11 | QSLEESGGRLVTPGTPLTLTCTVSGFSLSSY | AAVLTQTPSPVSAAVGGTV |
| | TVNWVRQAPGKGLEWIGYISYGGSAYYAS | SISCQSSQSVYNNNYLSWFQ |
| | WANGRFTISKTSATVDLKITSPTTEDTATY | QKPGQPPKLLIYGASTLTSG |
| | FCARHMQVGGAPTGSMAAFDPWGPGTLV | VPSRFKGSGSGTQFTLTISD |
| | TVSSGQPKAPSVFPLAPCCGDTPSSTVTLG | VQCDDAATYYCAGGYSGS |
| | CLVKGYLPEPVTVTWNSGTLTNGVRTFPS | SDKYAFGGGTEVVVKGDP |
| | VRQSSGLYSLSSVVSVTSSSQPVTCNVAHP | VAPTVLIFPPAADQVATGTV |
| | ATNTKVDKTVAPSTCSKPTCPPPELLGGPS | TIVCVANKYFPDVTVTWEV |
| | VFIFPPKPKDTLMISRTPEVTCVVVDVSQD | DGTTQTTGIENSKTPQNSAD |
| | DPEVQFTWYINNEQVRTARPPLREQQFNST | CTYNLSSTLTLTSTQYNSHK |
| | IRVVSTLPIAHQDWLRGKEFKCKVHNKAL | EYTCKVTQGTTSVVQSFNR |
| | PAPIEKTISKARGQPLEPKVYTMGPPREELS | GDC (SEQ ID NO: 32) |
| | SRSVSLTCMINGFYPSDISVEWEKNGKAED | |
| | NYKTTPAVLDSDGSYFLYNKLSVPTSEWQ | |
| | RGDVFTCSVMHEALHNHYTQKSISRSPGK | |
| | (SEQ ID NO: 33) | |

### Example 2: Distinct Toxicity Profiles for Inhibition of TGFβ2 and TGFβ3 with Isoform-Selective Anti-TGFβ Monoclonal Antibodies

Constitutive germline knockouts of TGFβ2 and TGFβ3 do not survive the perinatal period due to developmental defects including cleft palate in both and cardiovascular defects in TGFβ2 knockouts, and pan-TGFβ3 inhibitors have substantial toxicities as detailed above. To assess the potential toxicity of inhibiting TGFβ32, TGFβ33, or both, 4-week toxicology studies were conducted with 6F12, 2A10, and 4A11, as follows:

### TGFb3 (2A10):

In the pilot toxicology study, 2A10 mAb was administered thrice weekly (TIW) for 4 weeks (12 total doses) to CD-1 mice at doses of 0 (control), 10 and 50 mg/kg I.P. TIW, and 50 mg/kg I.V. TIW in a vehicle consisting of phosphate buffered saline. Males and females (n=5/sex/group) were assigned to the toxicity groups at all dose levels and were scheduled for necropsy at the end of the dosing period (Day 29). Blood for toxicokinetic evaluation was collected from additional mice in the control (n=6 males/group) and 2A10 mAb treated groups (n=9 males/group). Criteria for evaluations included the following parameters: clinical observations, body weight, clinical pathology (hematology and clinical chemistry), anatomic pathology, and toxicokinetics.

The 2A10 AUC exposures increased approximately dose-proportionally with an increase in dose from 10 to 50 mg/kg.

All animals survived to scheduled necropsy, and there were no 2A10-related clinical observations, effects on body weight, or clinical pathology changes. Findings attributed to 2A10 were limited to physeal dysplasia seen in a subset of mice at the high dose (6 mice at 50 mg/kg).

In conclusion, administration of 2A10 mAb to CD-1 mice at 10 and 50 mg/kg TIW for 4-weeks was well tolerated and 2A10-related finding were limited physeal dysplasia at the high dose.

### TGFb2 (6F12):

In the pilot toxicology study, 6F12 mAb was administered thrice weekly (TIW) for 4 weeks (12 total doses) to CD-1 mice at doses of 0 (control), 10, and 50 mg/kg I.P. in a vehicle consisting of phosphate buffered saline. Males and females (n=5/sex/group) were assigned to the toxicity groups at all dose levels and were scheduled for necropsy at the end of the dosing period (Day 29). Blood for toxicokinetic evaluation was collected from additional mice in the 6F12 mAb treated groups (n=9 males/group). Criteria for evaluations included the following parameters: clinical observations, body weight, clinical pathology (hematology and clinical chemistry), anatomic pathology, and toxicokinetics.

The 6F12 AUC exposures increased approximately dose-proportionally with an increase in dose from 10 to 50 mg/kg.

All animals survived to scheduled necropsy at 10 mg/kg and there were no 6F12-related clinical observations at 10 and 50 mg/kg.

6F12-related findings at 10 and 50 mg/kg included minimal to mild increase in neutrophils and lymphocytes in females with histopathology correlate of peritoneal inflammation indicative of chronic inflammation, and increase in ALT and AST with no histopathology correlate. Additional 6F12-related findings at 50 mg/kg included early euthanasia of 3 mice in the toxicokinetic group due to more than 20% decrease in body weights (observed on Day 8), as well as minimal to mild increases in globulin with corresponding decrease in albumin to globulin ratio in both sexes. All mice in the toxicity groups survived to scheduled necropsy on Day 29, and no changes in body weight were observed.

In conclusion, administration of 6F12 mAb to CD-1 mice at 10 and 50 mg/kg TIW for 4-weeks findings consistent with chronic inflammation were observed at 10 and 50 mg/kg.

### TGFb2/3 (4A11):

In the pilot toxicology study, 4A11 mAb was administered thrice weekly (TIW) for 4 weeks (12 total doses) to CD-1 mice at doses of 0 (control), 10 and 50 mg/kg I.P., and 50 mg/kg I.V. in a vehicle consisting of phosphate buffered saline. Males and females (n=5/sex/group) were assigned to the toxicity groups at all dose levels and were scheduled for necropsy at the end of the dosing period (Day 29). Blood for toxicokinetic evaluation was collected from additional mice in the control (n=6 males/group) and 4A11 mAb treated groups (n=9 males/group). Criteria for evaluations included the following parameters: clinical observations, body weight, clinical pathology (hematology and clinical chemistry), anatomic pathology, and toxicokinetics.

Key toxicology findings at 10 and 50 mg/kg included mortality in 3 mice (1 at 10 mg/kg and 2 at 50 mg/kg) and inguinal/scrotal hernias. Additional key findings at 50 mg/kg included clinical observation of hind limb loss of function pathology findings of perirectal serosal hemorrhage and hemoabdomen, periarteritis/arteritis and mural and interstitial hemorrhages in the cardiovascular (ascending aorta and myocardium) system, and degeneration and necrosis of the skeletal muscle.

In conclusion, administration of 4A11 mAb to CD-1 mice at 10 and 50 mg/kg TIW for 4-weeks led to mortality at 10 and 50 mg/kg and adverse pathology findings of hernias at all doses and in the cardiovascular system at 50 mg/kg.

The tox study findings are summarized in Table 8, below.

TABLE 8
Results of Toxicology Study

| Isoform | |
|---|---|
| Target | |
| (Clone) | Key Toxicology Findings |
| TGFβ2 | 10 mg/kg |
| (6F12) | Peritoneal inflammation with corresponding |
| | clinical pathology changes (increased neutrophils |
| | and lymphocytes, increased globulin) |
| | 50 mg/kg |
| | Peritoneal inflammation with corresponding |
| | clinical pathology changes (increased neutrophils |
| | and lymphocytes, increased globulin) |
| TGFβ3 | 10 mg/kg |
| (2A10) | No tox findings |
| | 50 mg/kg |
| | Physeal dysplasia (minor) |
| TGFβ2/3 | 10 mg/kg |
| (4A11) | Mortality |
| | Hernias - inguinal/scrotal |
| | Physeal dysplasia (minor) |
| | Hair follicle hyperplasia and dysplasia (minor) |
| | 50 mg/kg |
| | Mortality |
| | Hernias - inguinal/scrotal |
| | Bleeding - perirectal serosal hemorrhage, hemoabdomen |
| | Cardiovascular (ascending aorta and myocardium) - |
| | periarteritis/arteritis, mural and interstitial hemorrhages |
| | Skeletal muscle - degeneration and necrosis |
| | Hind limb loss of function |

### Example 3: Murine PK Study with Anti-TGFβ3 Monoclonal Antibodies

### Materials and Methods

Fab regions of the anti-TGFβ3 antibodies were fused to the Fc region of mouse IgG2a with a LALA-PG mutation to attenuate effector function for in vivo mouse studies (see Lo, M. et al. Journal of Biological Chemistry 292, 3900-3908 (2017); Schlothauer, T. et al. Protein Engineering Design and Selection 29, 457-466 (2016)).

### Results:

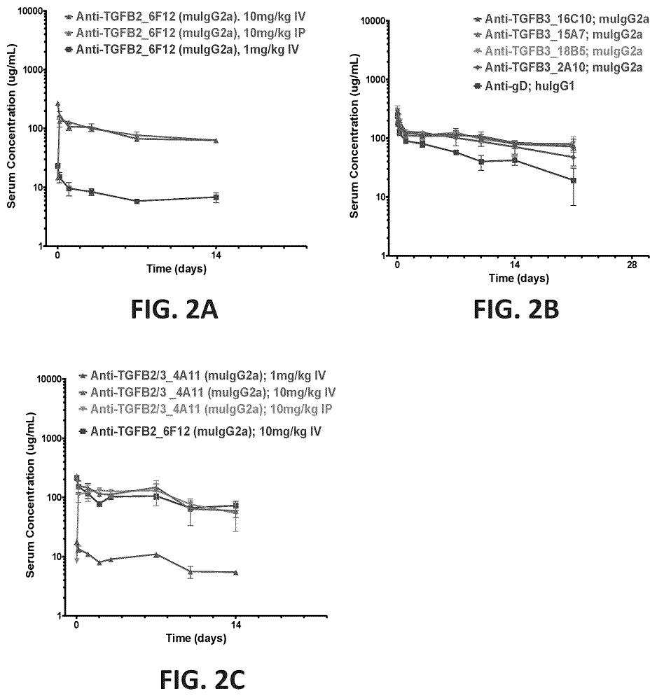

The Fab regions of the anti-TGFβ antibodies were fused to the Fc region of mouse IgG2a with a LALA-PG mutation to attenuate effector function for in vivo mouse studies. In C57/B6 mouse single-dose pharmacokinetic studies, these chimeric antibodies exhibited linear pharmacokinetics with dose dependent accumulation and no evidence of anti-drug antibody formation. The antibodies with the greatest in vitro potency (6F12 (anti-TGFβ2); 2A10 (anti-TGFβ3); and 4A11 (dual-specificity anti-TGFβ2/3) all exhibited in vivo bioavailability of ~95% and clearance of approximately 4 mL/day/kg, whereas 16C10, a slightly less potent anti-TGFβ3 antibody, exhibited clearance of approximately 2 mL/day/kg (FIG. 2).

### Example 4: TGFβ2 and TGFβ3 Expression Levels are Elevated in Human Fibrotic Tissue and TGFβ3 Expression is Highly Correlated with TGFβ R Signaling in IPF and SSc

### Materials and Methods

### Tissue Isolation (IPF) and RNA Analyses

RNA analyses of human tissues were described previously (Depianto, D J, et. al. Thorax. 2015 January; 70(1):48-56; Chandriani, S, et. al. J Immunol. 2014 Jul. 1; 193(1):111-9). Briefly, human tissues were obtained in the University of California, San Francisco Lung Center from patients with IPF at the time of biopsy or lung transplantation. Non-diseased normal lung tissues were procured from lungs not used by the Northern California Transplant Donor Network. RNA was isolated from the snap-frozen lung samples. Microarray analysis was done by following manufacturer's recommendations (Agilent, Santa Clara, CA).

### Derivation of a SSc Skin-Relevant TGFβ Gene Set and Signature Score:

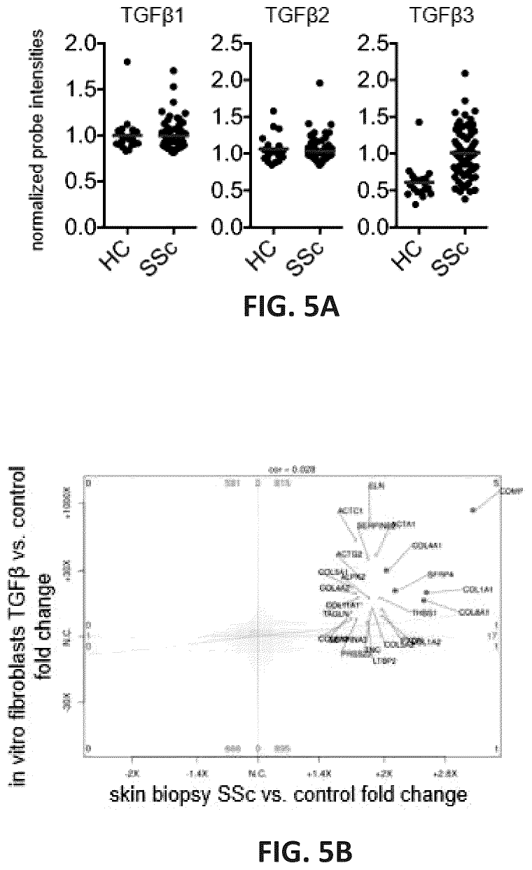

TGFβ-responsive genes were identified by performing RNA Sequencing on primary human lung fibroblasts stimulated with 5 ng/ml of human TGFβ1 (RnD systems) or control media for 24 hours. Sequencing data was processed using the R programming language and packages from the Bioconductor project (see World Wide Web at bioconductor.org) to express it at gene level as normalized reads per kilobase per million reads (nRPKM). A pilot SSc and normal control skin biopsy microarray dataset (GSE58095) was downloaded from NCBI Gene Expression Omnibus. To identify the set of genes that is upregulated in SSc skin and potentially attributable to TGFβ activity in fibroblasts, a 4-way comparison was made, contrasting the genes significantly dysregulated in vitro (≥4 fold increase by TGFβ) and in SSc vs HC (≥50% increase in disease). This candidate gene set is shown in FIG. 5B. The gene set was evaluated in the microarray data set from FaSScinate skin biopsies (see, Khanna et al. (2016) The Lancet; vol. 387:10038, p. 2630-2640 for description of the FaSSCinate study), and pruned to a core signature set of 18 genes as shown in Table 2, above, such that all genes contributed homogeneously to the first principal component (>0.7 for PCl loading). A composite gene expression score was defined for this gene set, using the first principal component loading, to reduce the aggregate expression of the 18-gene signature to a single continuous variable for each subject.

### ScRNAseq

### Preparation of Human Lung Tissue:

Explanted lung tissues were obtained from patients with a pathologic diagnosis of usual interstitial pneumonia and a consensus clinical diagnosis of IPF assigned by multidisciplinary discussion and review of clinical materials. After bronchoalveolar lavage, fresh lung explant tissue was stored in complete media on wet ice overnight. The tissue was washed in HBSS and then thoroughly minced in digestion buffer (HBSS, 2.5 mg/mL Collagenase D, 100 μg/mL DNase). Minced tissue was rocked 45 minutes at 37 °C. Residual tissue material was transferred into fresh digestion buffer and rocked another 45 minutes at 37 °C. Single cells from both rounds of digest were combined and utilized for downstream analyses. Remaining tissue was processed using a gentleMACS Dissociator (Miltenyi Biotec) to liberate additional cells into a single cell suspension. This preparation was then filtered and added to the previously isolated cells.

### FACS Isolation of Cell Populations:

Total number of cells were determined and resuspended in appropriate volume of 1×PBS supplemented with 2 mM EDTA and 2% fetal bovine serum (FBS). FcR blocking was performed using the Miltenyi Biotec FcR blocking reagent according to manufacturer's protocol. Cells were then stained with a cocktail of antibodies including CD45-BUV395, EPCAM-PE, CD31-BV605, and Live/Dead-Efluor 780 on ice for 30 minutes and then washed with supplemented PBS buffer 3 times. 5 populations of cells were collected for downstream scRNA-seq: Unsorted live cells, CD45₊, CD45⁻/EPCAM⁺, CD45⁻/CD31⁺, and CD45⁻/CD31⁻/EPCAM⁻(triple negative).

### Single-Cell RNA-Seq:

Single-cell RNA-seq was performed on the 10× Genomics platform using Chromium Single Cell 3′ Library and Gel bead kit v2 following manufacturer's user guide (10× Genomics). The cell density was used to impute the volume of single cell suspension needed in the reverse transcription (RT) master mix, aiming to achieve ~6,000 cells per sample. cDNAs and libraries were prepared following manufacturer's user guide (10× Genomics). Libraries were profiled by Bioanalyzer High Sensitivity DNA kit (Agilent Technologies) and quantified using Kapa Library Quantification Kit (Kapa Biosystems). Each library was sequenced in one lane of HiSeq4000 (Illumina) following manufacturer's sequencing specification (10× Genomics).

### Processing of Sequencing Data:

Cell Ranger v2.1.0 (10× Genomics, Pleasanton CA) was used to align raw sequencing reads against the GRCh38 human reference, count gene features, filter empty droplets, cluster cells, and identify cluster marker genes. Visualization of the tSNE results from Cell Ranger was performed using the Loupe Cell Browser v3.1 (10× Genomics, Pleasanton CA).

Immunohistochemistry for pSMAD3 and In Situ Hybridization for TGFβ3

Immunohistochemistry for phoshpho-SMAD3 was performed on formalin-fixed, paraffin-embedded sections using a rabbit monoclonal antibody (EP823Y, 0.76 pg/ml; Abcam). Briefly, sections were depariffinized and used for immunohistochemistry on an automated staining platform (Bond RX; Leica Biosystems) using proprietary reagents for antigen retrieval (ER2; Leica Biosystems), detection of bound primary antibody (anti-rabbit PowerVision polymer-HR; Leica Biosystems). Signal was visualized using DAB.

### Quantification of Serum Periostin and COMP Levels

Whole blood was collected from the patients and from 25 age- and gender-matched healthy controls through standard venipuncture technique in serum separator tubes, was allowed to clot for 30 minutes, and was spun down at 4 °C for 10 minutes. Serum was collected and stored at −70 °C until analyzed. Cartilage oligomeric matrix protein (COMP) levels were determined using IMPACT-based specific immunoassays (Roche Diagnostics, Penzberg, Germany). In brief, samples were incubated with an IMPACT chip coated with biotinylated COMP F(ab′)2 antibody fragments. After wash steps, biomarker-specific detection antibodies labelled with digoxigenin were added and incubated. After washing, fluorescence anti-digoxigenin-latex conjugate was added, and the specifically bound fluorescence label was detected with a charge-coupled detector camera. Emitted fluorescence was transformed into signal intensities using a standard curve composed of recombinant protein standards (R&D Systems, Minneapolis, MN, USA) and dedicated software (Roche Diagnostics). Sensitivity was 0·25 ng/mL for COMP. Periostin serum levels were determined using the clinical trial version of the Elecsys (Roche Diagnostics) periostin immunoassay intended for use on the cobas e601 (Roche Diagnostics).

### Results:

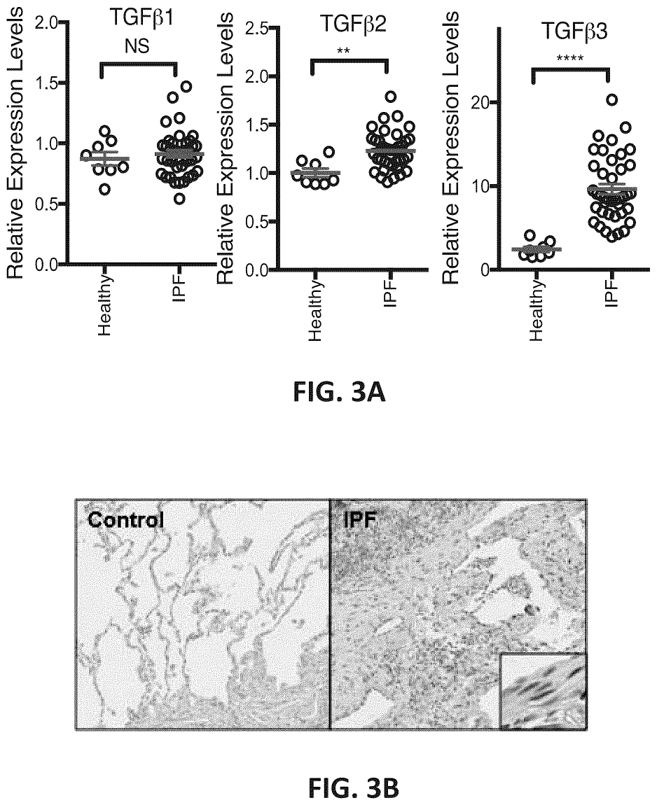

In qPCR analysis of bulk lung biopsy samples from patients with idiopathic pulmonary fibrosis (IPF), TGFβ3, and to a lesser extent, TGFβ2, were expressed at significantly higher levels compared to control lungs, whereas TGFβ1 expression was slightly lower on average in IPF compared to control (FIG. 3A). Immunohistochemistry analysis revealed abundant increases in nuclear phospho-SMAD2/3 staining in IPF tissue as compared to control, particularly in fibroblastic foci, regions of activated fibroblasts and myofibroblasts that are pathognomonic for a histological diagnosis of Usual Interstitial Pneumonia (UIP), the hallmark of IPF (FIG. 3B). As differences in gene expression in bulk IPF lung tissue may reflect cytological composition of the sample, it was next sought to characterize the cellular provenance of TGFβ isoforms on a single cell basis using single cell RNA sequencing (scRNAseq). Global gene expression patterns of scRNAseq data from IPF lungs distinguished multiple cell lineages including hematopoietic (macrophages, lymphocytes, plasmablasts, mast cells), epithelial (AEC2 and basal, ciliated, and secretory bronchial epithelial cells), mesenchymal (fibroblasts, myofibroblasts, smooth muscle), and vascular (endothelial) cell types.

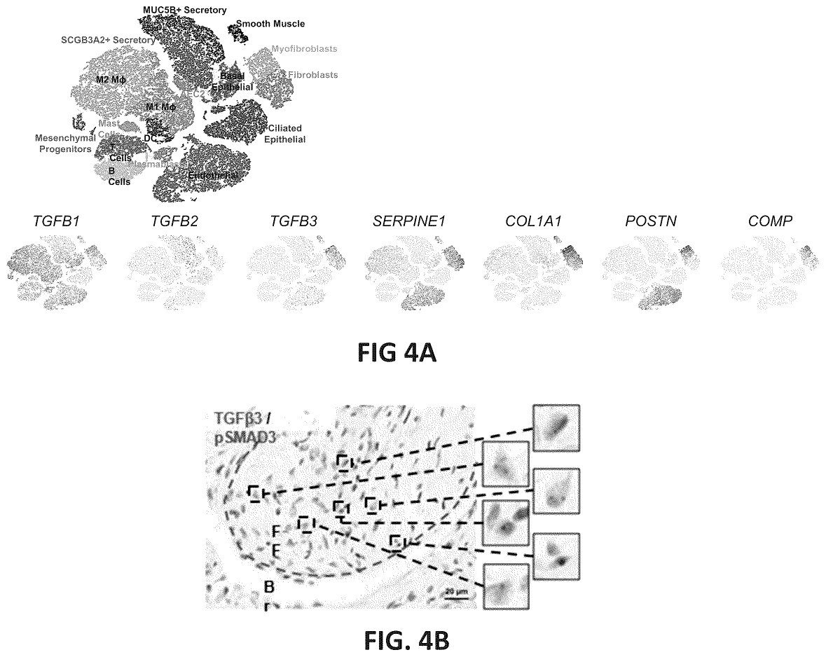

TGFβ1 was broadly expressed, with highest levels in hematopoietic cells, endothelial cells, and myofibroblasts. TGFβ2 was more restricted and primarily detected in epithelial derived cells. TGFβ3 expression was highly restricted to mesenchymal cells (fibroblasts, myofibroblasts, and smooth muscle cells). Expression of TGFβR-dependent target genes revealed distinct patterns, with SERPINE1 expressed in fibroblasts, macrophages, and endothelial cells, periostin (POSTN) in myofibroblasts and endothelial cells, and COL1A1 and COMP in myofibroblasts, largely overlapping with TGFβ3 (FIG. 4A). Dual immunohistochemistry for pSMAD3 and in situ hybridization for TGFβ3 revealed numerous myofibroblasts in fibroblastic foci co-expressing TGFβ3 and nuclear pSMAD3 (FIG. 4B), suggesting that in UIP lesions, TGFβ3 may be driving TGFβR-dependent signaling in an autocrine or paracrine fashion.

In skin biopsies taken at baseline in the FaSScinate trial, TGFβ3 expression is markedly upregulated in SSc patients as compared to healthy controls, whereas TGFβ1 and TGFβ2 levels are comparable (FIG. 5A). To assess the potential contribution of TGFβ signaling to differentially expressed genes in SSc skin, a gene set was derived that is both attributable to fibroblasts responding to TGFβ signaling and dysregulated in SSc skin. Given the striking heterogeneity in gene expression profiles known to exist in skin biopsies from SSc patients, it was hypothesized that at any given time, only a subset of the SSc cohort would manifest a high TGFβ activity profile.

Using the 18-gene TGFβ signature, a k-means clustering of SSc patients was performed and two clusters were identified—a “TGFβ-high” cluster that displays higher expression levels for many of the members of the gene set a “TGFβ-low” cluster, in which most of the members of the gene set registered a lower expression value.

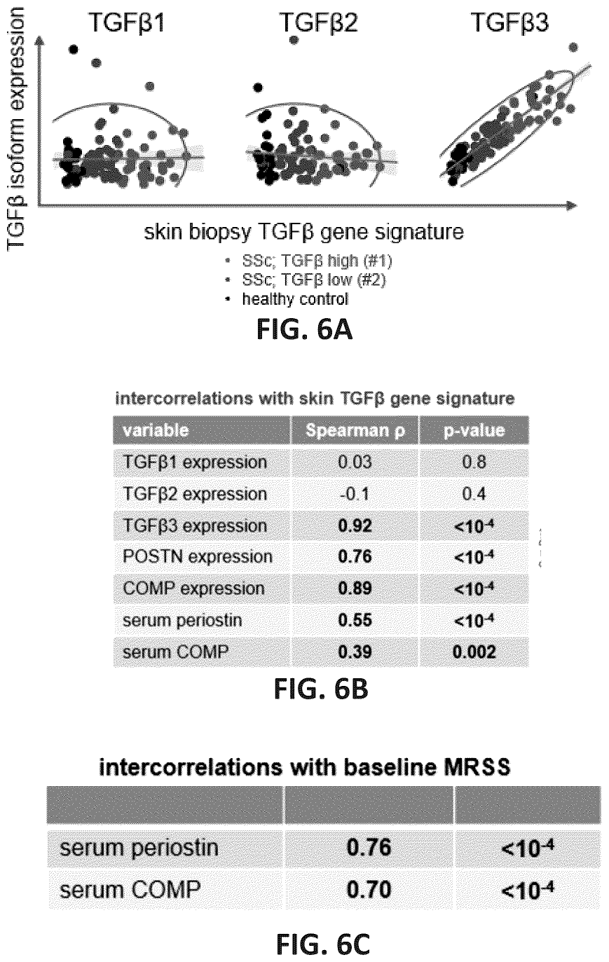

TGFβ3 expression correlated strongly and positively with the TGFβ-inducible signature (Spearman rho=0.92, p<0.0001), while the other two TGFβ isoforms did not correlate with the score (FIG. 6A and FIG. 6B).

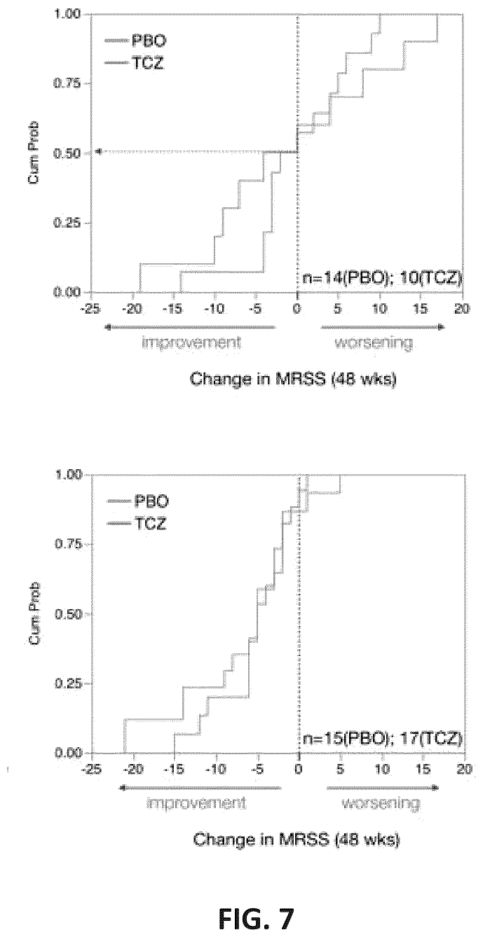

The TGFβ-inducible skin gene signature was also highly correlated with the expression levels of two genes encoding soluble secreted ECM components, POSTN and COMP. POSTN and COMP, the proteins encoded by these genes, are detectable in peripheral blood serum as soluble biomarkers and their levels correlated remarkably well with the skin biopsy TGFβ gene signature in contemporaneously collected blood samples (FIG. 6B). Furthermore, serum periostin and COMP levels were significantly correlated with the Modified Rodnan Skin Score (MRSS), a clinical index of systemic skin fibrosis used in SSc trials (FIG. 6C). To assess the relationship between skin TGFβ activity and disease progression, the change in MRSS over the 48-week course of the study was evaluated and it was found that TGFβ signature “high” patients were less likely to improve compared to TGFβ signature “low” patients regardless of treatment assignment (there was no significant benefit of tocilizumab vs. placebo on the MRSS outcome measure in the study; FIG. 7). Taken together, these data suggest that TGFβ3 expression and signaling activity in an isolated skin biopsy reflect overall disease burden and prognosis in SSc. Thus, these data support TGFβ3 inhibition as a target for the treatment of IPF and SSc, as well as the use of serum periostin and COMP protein levels or tissue TGFβ signature gene expression levels as biomarkers of TGFβ3 activity.

### Example 5: In Vivo Activity of Isoform-Selective Anti-TGFβ2 and TGFβ3 Antibodies in a Lung Fibrosis Model

### Materials and Methods

### Bleomycin Instillation

For the bleomycin study, adult mice (>12 weeks) were randomized based on their weights before the study to minimize variance between experimental and control groups. For intratracheal (I.T.) dosing, all mice were lightly anesthetized with isoflurane in an induction chamber. Once anesthetized, the animals were removed from the chamber, manually restrained, the mouth of the animal was opened and the tongue set aside. A 1 ml syringe with 50 microliters of sterile injectable isotonic saline or bleomycin (0.70 U/kg (DNC #0703-3155-01; TEVA) in 50 μl sterile isotonic saline) was connected to a 24 gauge gavage needle. The gavage needle was inserted into the trachea and a dose of either vehicle or bleomycin was delivered intratracheally. After delivery, animals were monitored continuously until fully awake and ambulatory.

For profibrotic gene expression analysis, animals were taken down 14 days post bleomycin delivery. RNA was harvested from whole mouse lungs and the gene expression was determined by RT-qPCR.

To measure the collagen deposition in the lung, mice were taken down 24 days after bleomycin administration. Deuterated water labeling was used to assess the new collagen synthesis in studies (Blaauboer M E, et. al. Matrix Biol. 2013; 32(7-8):424-31.). In our studies, the labeling was started at 9th day after bleomycin delivery, by I.P. injecting deuterated water (Cat #DLM-4-99.8-1000; Cambridge Isotope Laboratories) 35 ml/kg in 2 divided doses 4 hours apart. Afterward, 8% deuterated water in drinking water was provided ad lib in a water bottle until the end of the study.

Deuterated water incorporation into hydroxyproline was analyzed as described previously (Gardner J L, et.al. Am J Physiol Gastrointest Liver Physiol. 2007; 292(6):G1695-705). Mass spectrometry and analysis were performed by Metabolic Solutions. New hydroxyproline content was expressed as “pg per lung”.

### Gene Expression

Gene expression analyses in the bleomycin induced mouse lung fibrosis model were done by analyzing a published data set, which can be accessed through GEO Series accession number GSE37635 (see World Wide Web @ncbi.nlm.nih.gov/geo/query/acc.cgi?acc=GSE37635) (see, also, Blaauboer, 2013, MatrixBiol 32, 424-431).

### RNA Sequencing

To perform the RNAseq study on different lung cell populations in a mouse bleomycin model, mouse whole lungs were harvested at day 14 after bleomycin administration. There were 5 mice in the saline control group, and 8 mice in the bleomycin group. Single cells were isolated after the protease digestion by following our previously published protocol (Sun T, et al., 2019, JCI Insight; June 18; 5(14):e128674). After washing, cells were blocked by purified rat anti-mouse CD16/CD32 antibody (Cat #553142; BD Biosciences) and stained with antibodies for CD45 (Cat #17-0451-82, eBioscience), EpCAM (Cat #11-5791-82, eBioscience), and CD31 (Cat #25-0311-82, eBioscience). Four cell populations from each mouse were sorted based on their cell surface markers: leukocytes (CD45⁺; EpCAM⁻); epithelial cells (CD45⁻, EpCAM⁺); endothelial cells (CD45⁻; EpCAM⁻; CD31⁺) and fibroblasts (triple negatives). RNA was purified from the sorted cells using the RNeasy Mini Kit (Cat #74106; Qiagen). 0.5 pg of total RNA was used as an input material for library preparation using TruSeq RNA Sample Preparation Kit v2 (Illumina, San Diego CA), which was quality controlled with Fragment Analyzer and a Library quantification kit (KAPA Biosystems, Wilmington MA). The libraries were multiplexed and then sequenced on Illumina HiSeq2500 to generate 30M of single end 50 base pair reads. Sequencing reads were mapped to the reference mouse genome (GRCm38), using the GSNAP short read aligner (Wu and Nacu, 2010), and assigned to RefSeq gene models. Expression was measured in normalized reads per kilobase per million total reads (nRPKM).

### Conditional Knockouts

Two targeting constructs were targeting the exon 6s in Tgfb2 or Tgfb3 locus respectively. Adeno-FLP was used to treat positive ES clones to remove the PGK neomycin selection marker at the ES cell stage. Two loxP sites were created to flank the exon 6 in the TGFβ2 or β loci in their respective cKO mice.

qPCR

Lung genomic DNAs were isolated from tamoxifen treated mice and evaluated by quantitative PCR (vs. Tert). Exon 3 is located outside of the floxed locus and was used as the experimental control.

### Results:

Intratracheal (I.T.) bleomycin challenge is a frequently used in vivo rodent model of lung fibrosis. Bleomycin selectively injures alveolar epithelial cells, triggering an innate immune response followed by fibroblast differentiation into myofibroblasts and ECM deposition. Unlike a chronic, progressive condition such as IPF, the bleomycin model has specific phases: injury, inflammation, fibrosis, and, in young animals, resolution. To determine whether TGFβ isoform expression was relevant in this model and during which phases, lung gene expression in mice was assessed at various time points after i.t. bleomycin instillation.

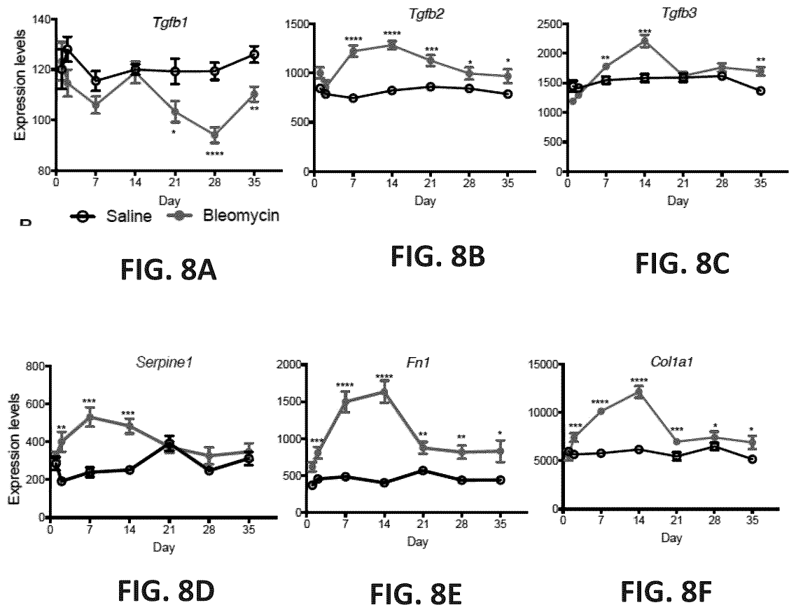

To assess the kinetics of TGFβ isoform expression in this model, lung gene expression was assessed in mice at various timepoints after i.t. bleomycin instillation based on a published transcriptomic dataset (Blaauboer, supra). While Tgfb1 expression was not substantially elevated in bleomycin-challenged mice at any time point, and was in fact slightly decreased at days 21 and 28, Tgfb2 and Tgfb3 expression were substantially increased after bleomycin installation at day 7 and day 14 after challenge (FIG. 8A, FIG. 8B, FIG. 8C). This pattern overlapped well with the induction of genes associated with fibrogenesis and TGFβ signaling, such as Serpine1, Fn1, and Col1a1 (FIG. 8D, FIG. 8E, FIG. 8F).

The significant upregulation of Tgfb2 and Tgfb3 during the fibrotic phase of the model concurred with expression pattern observed in IPF lung (FIG. 3A). To further characterize the specific cell types responsible for the expression of each TGFβ isoform in this model, RNAseq was performed on sorted lung cell populations day 14 after bleomycin administration when multiple fibrosis-associated genes peaked. Consistent with scRNAseq data from human IPF lung tissue (FIG. 4A), leukocytes (CD45⁺) had the highest Tgfb1 expression levels. Tgfb2 and Tgfb3 were predominantly expressed in fibroblasts (CD45⁻, EpCAM⁻ CD31⁻); Tgfb2 was also induced in epithelial cells and TGFβR-inducible genes Col1a1 and Serpine1 were induced mainly in fibroblasts after bleomycin treatment. While substantial upregulation of Tgfb3 was not observed in any individual cell populations after bleomycin treatment, the significant upregulation in Tgfb3 in bulk lung tissue (FIG. 8A, FIG. 8B, FIG. 8C) was attributed to increased proportions of Tgfb3-expressing fibroblasts at those timepoints.

As constitutive deletion of Tgfb2 or Tgfb3 results in embryonic or perinatal lethality, inducible knockouts of these genes were generated to investigate their roles in bleomycin-induced lung fibrosis in adult animals, by introducing loxP sites in Tgfb2 (Tgfb2^{fl/fl}) and Tgfb3 (Tgfb3^{fl/fl}), respectively. Tgfb2^{fl/fl}, Tgfb3^{fl/fl}, and Tgfb2^{fl}/Tgfb3^{fl/fl} were crossed to ROSA-CreER^{T2}, and 12-16 week old mice were dosed with tamoxifen to delete the respective TGFβ alleles (single conditional KO mice will be referred to as TGFβ2.cKO and TGFβ3.cKO, and double targeted mice will be referred to as TGFβ2/3.cDKO). No gross abnormalities or morbidities were observed in cKO animals up to 6 weeks after tamoxifen treatment. To assess the roles of TGFβ2 and TGFβ3 in lung fibrosis, adult cKO animals were treated with tamoxifen starting 2 weeks before bleomycin administration. From days 9-24 after bleomycin administration, deuterated drinking water was administered to enable determination of new collagen synthesis in whole lungs by measuring deuterated hydroxyproline. In both TGFβ2.cKO and TGFβ3.cKO mice, new collagen synthesis was significantly reduced, as well as in TGFβ2/3.cDKO mice, suggesting that both TGFβ2 and TGFβ3 contribute to fibrogenesis in this model (FIG. 52A and FIG. 52B).

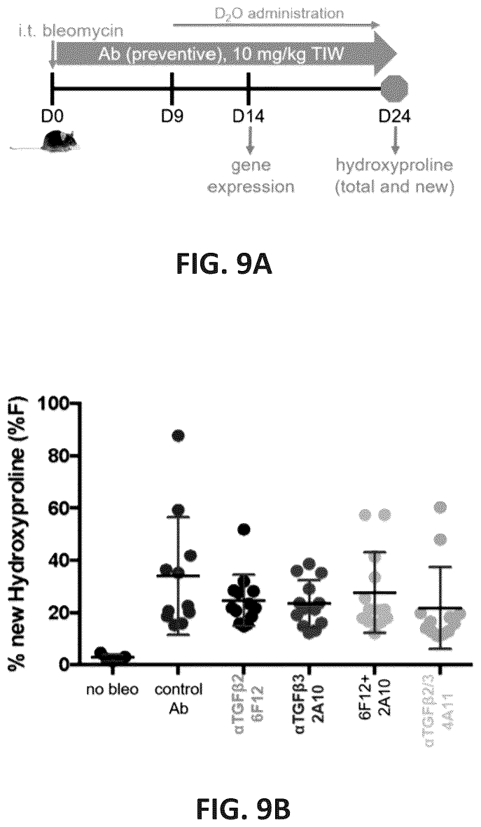

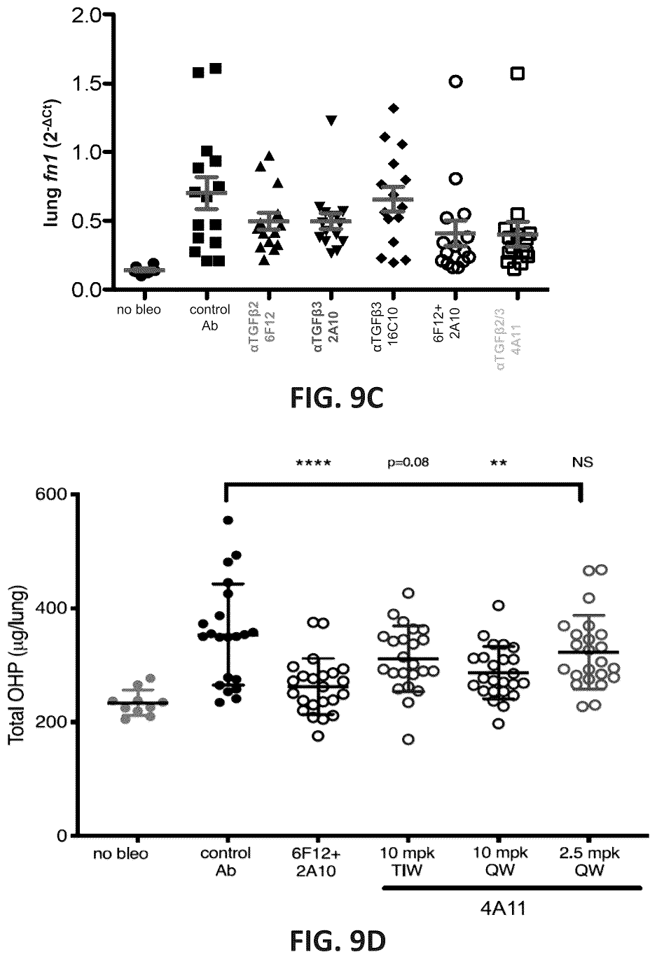

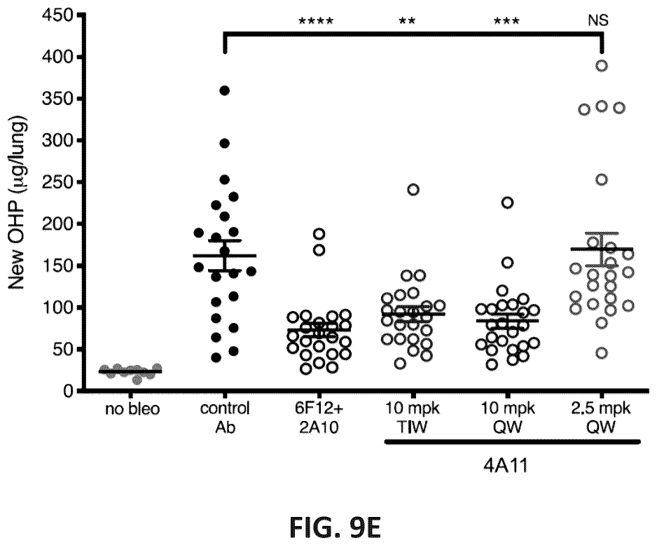

The significant upregulation of TGFβ2 and TGFβ3 during the “fibrotic” phase of the model mirrored the isoform distribution that was observed in IPF lung. Therefore, TGFβR-induced target gene expression at day 14 and collagen accumulation at day 24 were prioritized as key readouts in the context of preventive treatment with anti-TGFβ antibodies over the course of the study to assess their in vivo activity and potency (schematic, FIG. 9A).

When dosed at 10 mg/kg three times per week, single inhibition of TGFβ2 with 6F12, TGFβ3 with 2A10, or both with either the combination of 6F12+2A10 or monotherapy with 4A11 reduced new collagen synthesis reflected in the incorporation of deuterium into lung hydroxyproline in animals administered D₂O in drinking water from day 9-23 after bleomycin (FIG. 9B). As a more proximal pharmacodynamic readout, in a separate cohort of animals, lung gene expression of fibronectin-1 (fn1) at day 14 after bleomycin was significantly reduced by 6F12, 2A10, the combination of 6F12+2A10, or 4A11. However, 16C10, a less potent TGFβ3 antibody, had no effect on lung fn1 expression (FIG. 9C). Similar effects were observed on other TGFβ-inducible genes including serpine1, col1a1, col1a2, and col3a1. While single administration of either anti-TGFβ2 antibody or anti-TGFβ3 antibody reduced gene expression and collagen synthesis, the combination of both via co-administration of 6F12+2A10 or single administration of 4A11 was numerically superior across multiple experiments, suggesting that both TGFβ2 and TGFβ3 contribute to fibrogenesis in this model, and that the 6F12, 2A10, and 4A11 antibodies can inhibit TGFβ isoforms in vivo (FIG. 9D and FIG. 9E).

### Example 6: Humanization of anti-TGFβ3 mAb 2A10

### Materials and Methods

### Antibody Affinity Measurement by BIAcore

For binding affinity determinations of all humanized 2A10 antibodies, Surface Plasmon Resonance (SPR) measurement with a BIAcore™-T200 instrument was used. Briefly, series S CM5 biosensor chip with protein A was first applied to capture each humanized 2A10 variant to achieve approximately 100 response units (RU) on each flow cell, following by injection of 3-fold serial dilutions of human TGFβ1, TGFβ2 or TGFβ3 (0.02 nM to 5 nM) in HBS-EP buffer (0.01M HEPES pH 7.4, 0.15M NaCl, 3 mM EDTA and 0.05% v/v surfactant P20) at 25 °C with a flow rate of 50 μl/min for kinetics measurement. Association rates (kₒₙ) and dissociation rates (k_{off}) were calculated using a simple one-to-one Langmuir binding model (BIAcore T200 evaluation software version 2.0) to determine the equilibrium dissociation constant (KD), which was calculated as the ratio k_{off}/kₒₙ.

### HEKBlue Cell-Based TGFβ Blocking Assay

HEK-Blue™ TGFβ cells (InvivoGen, San Diego, CA) stably expressing human TGFβRI, Smad3, Smad4 proteins and secreted alkaline phosphatase (SEAP) reporter gene under the control of the β-globin minimal promoter fused to three Smad3/4-binding elements (SBE) were used to evaluate the blocking potency of 2A10. 2A10 was incubated with each of the TGFβ isoforms (PeproTech, Rocky Hill, NJ) at 25 °C for 1 h prior to the addition into 4.5×104 HEK-Blue™ TGF-β cells seeded in 96-well plate in 100 μL of assay buffer (DMEM high glucose with 10% heat inactivated FBS, 2 mM L-glutamine and 0.5% Penicillin Streptomycin). The final concentration of antibody and TGFβ was 0.001-200 nM and 20 pM respectively. The plate was incubated at 37 °C and 5% CO₂ for 18-22 h. The SEAP level in the cell supernatant was determined by QUANTI-Blue™ assay according to the manufacturer's instructions (InvivoGen). Pan-TGFβmAb neutralizes TGFβ1, 2, and 3 at low cellular potencies (IC50) of 0.72, 2.2, and 0.026 nM, respectively.

### Results:

### Humanization of 2A10:

2A10 Humanization Step 1: Rat 2A10 CDRs Grafting into Human Frameworks:

Monoclonal antibody 2A10 was humanized as described below. Residue numbers are according to Kabat et al. (Sequences of proteins of immunological interest, 5th Ed., Public Health Service, National Institutes of Health, Bethesda, MD (1991)). For 2A10 antibody humanization, hypervariable regions from the rat 2A10 (r2A10) antibody were engineered into human germline IGKV1D-39 and IGHV3-49 acceptor frameworks to generate humanized 2A10 version 1 (h2A10.v1) and human germline IGKV1D-39 and IGHV3-23 acceptor frameworks to generate humanized 2A10 version 2 (h2A10.v2). Specifically, from the r2A10 light chain variable domain (VL), positions 24-34 (L1), 50-56 (L2) and 89-97 (L3) were grafted into IGKV1D-39. From the r2A10 heavy chain variable domain (VH), positions 26-35 (H1), 50-65 (H2) and 95-102 (H3) were grafted into IGHV3-49 and IGHV3-23 respectively.

In addition, position 4 in framework I, position 38 and 43 in framework II, position 58 in framework III of VL, position 47 and 49 in framework II and position 78 in framework III of VH were retained from the rat sequence in h2A10.v1. Like h2A10.v1, two additional positions (73 and 76) in framework III of VH were also retained from the rat sequence in h2A10.v2. Those residues were found to be part of the framework residues acting as “Vernier” zone, which may adjust CDR structure and fine-tune the antigen fit. See, e.g., Foote and Winter, J. Mol. Biol. 224: 487-499 (1992). These CDR definitions include positions defined by their sequence hypervariability (Wu, T. T. & Kabat, E. A. (1970)), their structural location (Chothia, C. & Lesk, A. M. (1987)) and their involvement in antigen-antibody contacts (MacCallum et al. J. Mol. Biol. 262: 732-745 (1996)).

Both primary humanized versions were subject to human TGFβ isoforms binding in BIAcore and the results indicated h2A10.v2 is more comparable to r2A10 than h2A10.v1 in retaining human TGFβ3 binding (Table 9). Interestingly, after humanization, both humanized versions showed human TGFβ2 binding drop in comparison with r2A10.

### 2A10 Humanization Step 2: Framework Polishing:

In humanized 2A10 framework polishing, more humanized 2A10 variants were generated by substituting each rat residue of humanized 2A10 at Vernier zone with corresponding human residue from germline sequence. Each variant was then evaluated by human TGFβ isoforms binding in BIAcore and blocking potency in HEKBlue to elucidate its importance.

In h2A10.v1, these framework polishing variants are h2A10.v1.1 (VL: L4M), h2A10.v1.2 (VL: H38Q), h2A10.v1.3 (VL: Q43A), h2A10.v1.4 (VL: I58V), h2A10.v1.5 (VH: L47W), h2A10.v1.6 (VH: A49G), h2A10.v1.7 (VH: V78A) (Table 9). In purification, some of the framework polishing variants in h2A10.v1 tend to have expression (h2A10.v1.1) and aggregation (Monomer <95% for h2A10.v1.3, h2A10.v1.4, h2A10.v1.6) issues. Therefore, it was decided to move on with h2A10.v2 variants.

In h2A10.v2, these framework polishing variants are h2A10.v2.1 (VL: L4M), h2A10.v2.2 (VL: H38Q), h2A10.v2.3 (VL: Q43A), h2A10.v2.4 (VL: I58V), h2A10.v2.5 (VH: L47W), h2A10.v2.6 (VH: A49S), h2A10.v2.7 (VH: D73N), h2A10.v2.8 (VH: S76N), h2A10.v2.9 (VH: V78L). Unlike h2A10.v1 variants, all h2A10.v2 variants were expressed and behaved well in purification (Monomer >99%). In BIAcore binding study, most of variants appeared to be the same as parental h2A10.v2, except for h2A10.v2.1 (VL: L4M) and h2A10.v2.7 (VH: D73N) variants with slightly drop in TGFβ3 binding (2-3 fold) and h2A10.v2.5 (VH: L47W) variant with more drastically drop in TGFβ3 binding (385 fold) (Table 9). Surprisingly, in the cellular HEKBlue TGFβ blocking study, both h2A10.v2.5 (VH: L47W) and h2A10.v2.7 (VH: D73N) showed significant loss of TGFβ3 blocking (>10-fold), but not for h2A10.v2.1 (VL: L4M) (Table 9). Overall, it was determined that all rat residues at Vernier zone of h2A10.v2 can be replaced with human germline residues, except for position 4 in framework I of VL, position 47 in framework II and position 73 in framework III of VH.

TABLE 9
h2A10.v1 and h2A10.v2 framework polishing variants characterization summary

| | | | | | | HEKBIueTGFp | | | SEC |
|---|---|---|---|---|---|---|---|---|---|
| 2A10 | Human | Framework | BIAcore (pM) | | | blocking IC50 (nM) | | | Monomer |
| variants | Framework | mutation | TGFβ1 | TGFβ2 | TGFβ3 | TGFβ1 | TGFβ2 | TGFβ3 | (%) |
| Rat 2A10 | — | — | >5000 | 62.8 | 1.2 | >200 | 70 | 0.01 | — |
| h2A10.v1 | K_{1D-39}H₃₋₄₉ | — | — | — | — | — | — | — | 88.9 |
| h2A10.v1.1 | K_{1D-39}H₃₋₄₉ | LC: L4M | No expression | | | | — | — | — |
| h2A10.v1.2 | K_{1D-39}H₃₋₄₉ | LC: H38Q | — | — | — | — | — | — | 97.7 |
| h2A10.v1.3 | K_{1D-39}H₃₋₄₉ | LC: Q43A | — | — | — | — | — | — | 92.0 |
| h2A10.v1.4 | K_{1D-39}H₃₋₄₉ | LC: I58V | — | — | — | — | — | — | 90.1 |
| h2A10.v1.5 | K_{1D-39}H₃₋₄₉ | HC: L47W | — | — | — | — | — | — | 98.6 |
| h2A10.vl.6 | K_{1D-39}H₃₋₄₉ | HC: A49G | — | — | — | — | — | — | 94.0 |
| h2A10.v1.7 | K_{1D-39}H₃₋₄₉ | HC: V78A | — | — | — | — | — | — | 96.5 |
| h2A10.v2 | K_{1D-39}H₃₋₂₃ | — | >5000 | 314 | 1.3 | >200 | >200 | 0.01 | 99.6 |
| h2A10.v2.1 | K_{1D-39}H₃₋₂₃ | LC: L4M | >5000 | 238 | 3.1 | >200 | >200 | 0.01 | 99.4 |
| h2A10.v2.2 | K_{1D-39}H₃₋₂₃ | LC: H38Q | >5000 | 124 | 1.2 | >200 | >200 | 0.01 | 99.5 |
| h2A10.v2.3 | K_{1D-39}H₃₋₂₃ | LC: Q43A | >5000 | 242 | 1.5 | >200 | >200 | 0.01 | 99.5 |
| h2A10.v2.4 | K_{1D-39}H₃₋₂₃ | LC: I58V | >5000 | 190 | 2.1 | >200 | >200 | 0.01 | 99.5 |
| h2A10.v2.5 | K_{1D-39}H₃₋₂₃ | HC: L47W | >5000 | 2820 | 500 | >200 | >200 | 27.4 | 99.6 |
| h2A10.v2.6 | K_{1D-39}H₃₋₂₃ | HC: A49S | >5000 | 249 | 1.5 | >200 | >200 | 0.01 | 99.8 |
| h2A10.v2.7 | K_{1D-39}H₃₋₂₃ | HC: D73N | >5000 | 921 | 3.2 | >200 | >200 | 0.12 | 99.5 |
| h2A10.v2.8 | K_{1D-39}H₃₋₂₃ | HC: S76N | >5000 | 365 | 1.2 | >200 | >200 | 0.01 | 99.6 |
| h2A10.v2.9 | K_{1D-39}H₃₋₂₃ | HC: V78L | >5000 | 768 | 0.7 | >200 | >200 | 0.01 | 99.6 |

2A10 Humanization Step 3: Fix CDR-H2 N-Linked Glycosylation Site (N⁵⁴Y⁵⁵P⁵⁶)

In h2A10.v2, a potential N-linked glycosylation site (N⁵⁴Y⁵⁵T⁵⁶) in CDR-H2 was identified as a potential liability. Two approaches were applied to address this issue, one of which was to substitute Asn at position 54 to nucleophilic Ser (h2A10.v2.N54S) or its homolog Gln (h2A10.v2.N54Q), the other of which was to keep Asn at position 54, but substitute Thr at position 56 to a smaller residue Ala (h2A10.v2.T56A). All three variants were expressed and behaved well in purification (Monomer >990%) (Table 10). In BIAcore analysis, however, only the h2A10.v2.T56A variant retained similar human TGFβ3 binding while the other two variants showed a slight drop (<2-fold) by comparison with parental h2A10.v2. Despite the differences in binding, however, no differences were observed with respect to human TGFβ3 blocking for all three variants.

TABLE 10
h2A10.v2 CDR-H2 N⁵⁴Y⁵⁵T⁵⁶ variants characterization summary

| | | | | | | HEKBlue TGFβ | | | SEC |
|---|---|---|---|---|---|---|---|---|---|
| 2A10 | Human | Framework | BIAcore (pM) | | | blocking IC50 (nM) | | | Monomer |
| variants | Framework | mutation | TGFβ1 | TGFβ2 | TGFβ3 | TGFβ1 | TGFβ2 | TGFβ3 | (%) |
| h2A10.v2 | K_{1D-39}H₃₋₂₃ | — | >5000 | 314 | 1.3 | >200 | >200 | 0.01 | 99.6 |
| h2A10.v2.N54S | K_{1D-39}H₃₋₂₃ | — | >5000 | 425 | 2.4 | >200 | >200 | 0.01 | 99.5 |
| h2A10.v2.N54Q | K_{1D-39}H₃₋₂₃ | — | >5000 | 378 | 2.5 | >200 | >200 | 0.01 | 99.6 |
| h2A10.v2.T56A | K_{1D-39}H₃₋₂3 | — | >5000 | 247 | 1.0 | >200 | >200 | 0.01 | 99.6 |

### 2A10 Humanization Step 4: Final Version Generation

By combining the results from framework polishing and CDR-H2 N-linked glycosylation site repair, the final two humanized 2A10 versions, h2A10.v3 and h2A10.v4, were generated based on further engineering of h2A10.v2. Both versions retained L at position 4 in framework I of VL and L at position 47 in framework II, D at position 73 in framework III of VH from r2A10. The other six framework residues derived from r2A10 at Vernier zone were changed to corresponding human germline residues, which were Q at position 38 and A at position 43 in framework II, V at position 58 in framework III of VL and S at position 49 in framework II, N at position 76 and L at position 78 in framework III of VH. The only difference between the two versions were at CDR-H2 N-linked glycosylation site, which were S⁵⁴Y⁵⁵T⁵⁶ in h2A10.v3 and N⁵⁴Y⁵⁵T⁵⁶ in h2A10.v4. This difference was significant functionally, however, because in BIAcore TGFβ3 binding and HEKBlue TGFβ3 blocking assays, h2A10.v4 appeared to be better than h2A10.v3 and comparable to parental r2A10 (Table 11).

Overall, the humanization process for 2A10 was quite challenging. The challenges in rat-derived 2A10 humanization included unexpected difficulty with antibody expression at the 1^{st} step (grafting 2A10 CDRs onto the closest human VH/VL frameworks pairing) and with stability issues of the variants in the 2^{nd} step of framework polishing (see FIG. 10, FIG. 11).

TABLE 11
h2A10.v3 and h2A10.v4 characterization summary

| | | | | | | HEKBlue TGFβ | | | SEC |
|---|---|---|---|---|---|---|---|---|---|
| 2A10 | Human | Framework | BIAcore (pM) | | | blocking IC50 (nM) | | | Monomer |
| variants | Framework | mutation | TGFβ1 | TGFβ2 | TGFβ3 | TGFβ1 | TGFβ2 | TGFβ3 | (%) |
| Rat2A10 | — | — | >5000 | 62.8 | 1.2 | >200 | 70 | 0.01 | — |
| h2A10.v3 | K_{1D-39}H₃₋₂₃ | LC: H38Q, Q43A | >5000 | 482 | 2.6 | >200 | >200 | 0.05 | 99.7 |
| | | I58V | | | | | | | |
| | | HC: A49S, S76N | | | | | | | |
| | | V78L | | | | | | | |
| h2A10.v4 | K_{1D-39}H₃₋₂₃ | LC: H38Q, Q43A | >5000 | 300 | 1.9 | >200 | >200 | 0.01 | 100 |
| | | I58V | | | | | | | |
| | | HC: A49S, S76N | | | | | | | |
| | | V78L | | | | | | | |

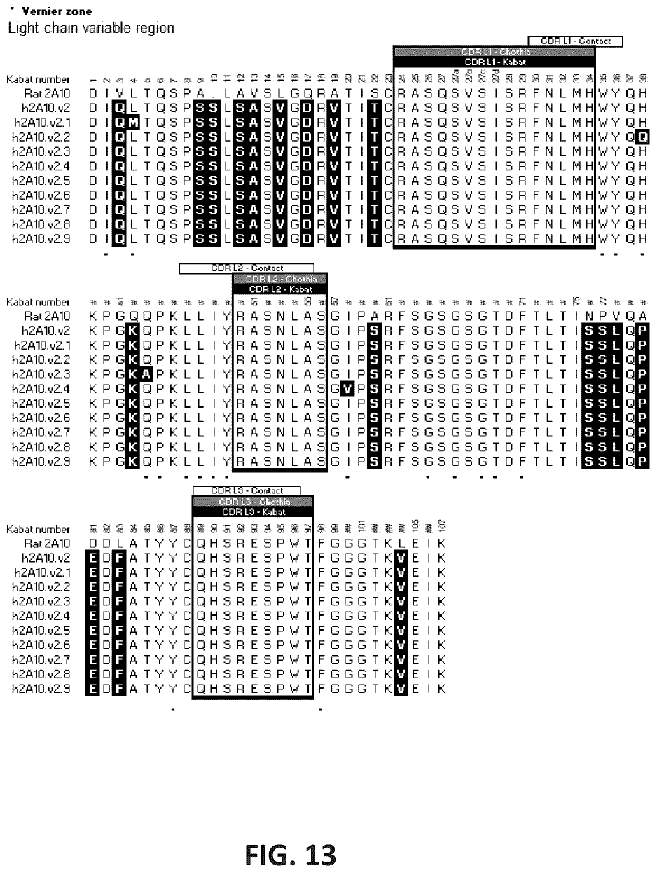

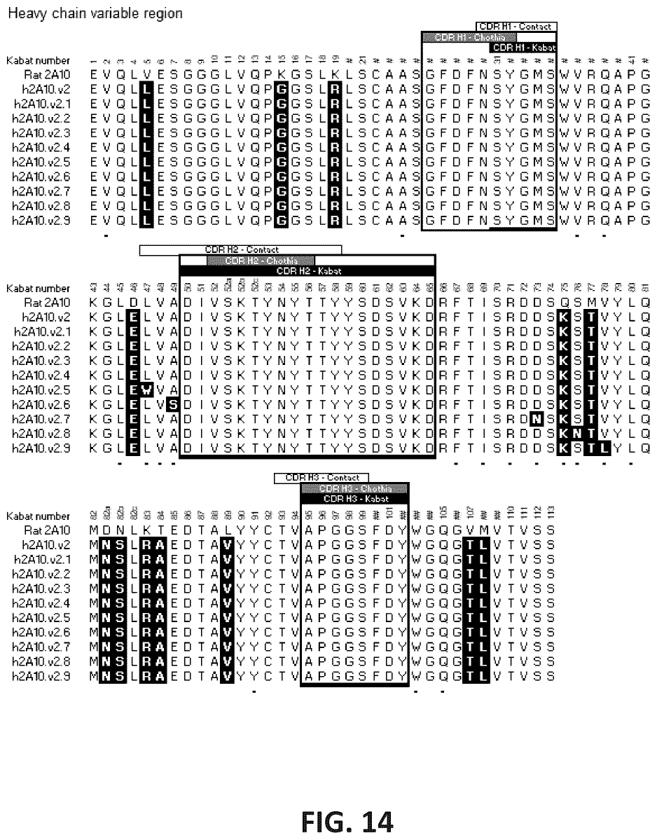

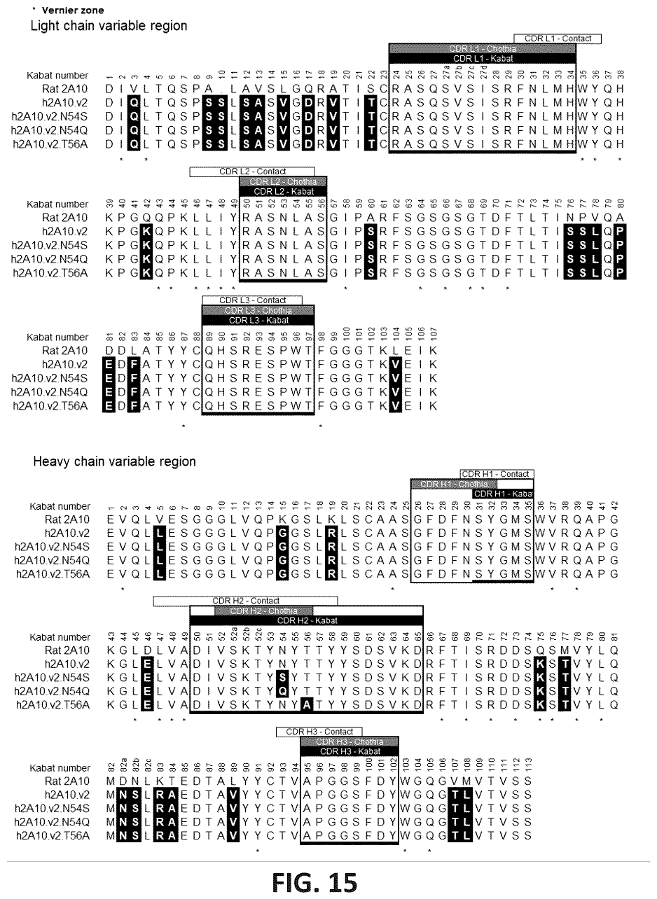

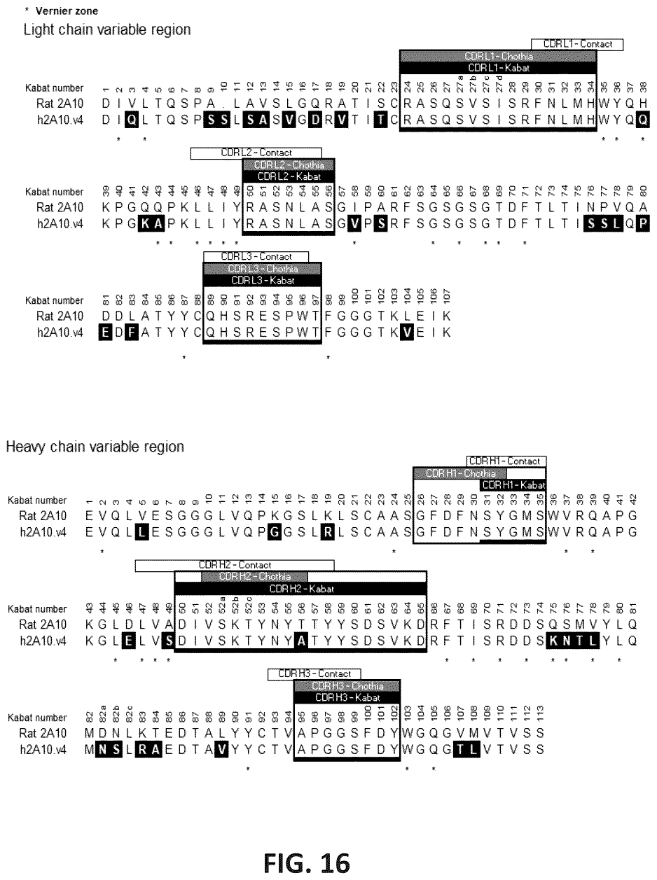

The sequences for humanized 2A10 antibody variants (CDRs of select variants, variable regions, and complete sequences) are shown in Table 12, Table 13, and Table 14, below, and sequence alignments are shown in FIG. 12, FIG. 13, FIG. 14, FIG. 15, and FIG. 16.

TABLE 12
Heavy and Light Chain CDR Amino Acid Sequences of Humanized 2A10
Variants

| | CDR-H1 | CDR-H2 | CDR-H3 | CDR-L1 | CDR-L2 | CDR-L3 |
|---|---|---|---|---|---|---|
| H2A10.v2.N54Q | SYGMS | DIVSKTYQ | APGGSFD | RASQSVS | RASNLAS | QHSRES |
| | (SEQ ID | YTTYYSDS | Y | ISRFNLM | (SEQ ID | PWT |
| | NO: 4) | VKD | (SEQ ID | H | NO: 8) | (SEQ ID |
| | | (SEQ ID | NO: 6) | (SEQ ID | | NO: 9) |
| | | NO: 159) | | NO: 7) | | |
| h2A10.v3 | SYGMS | DIVSKTYS | APGGSFD | RASQSVS | RASNLAS | QHSRES |
| | (SEQ ID | YTTYYSDS | Y | ISRFNLM | (SEQ ID | PWT |
| | NO: 4) | VKD | (SEQ ID | H | NO: 8) | (SEQ ID |
| | | (SEQ ID | NO: 6) | (SEQ ID | | NO: 9) |
| | | NO: 34) | | NO: 7) | | |
| h2A10.v4 | SYGMS | DIVSKTYN | APGGSFD | RASQSVS | RASNLAS | QHSRES |
| | (SEQ ID | YATYYSDS | Y | ISRFNLM | (SEQ ID | PWT |
| | NO: 4) | VKD | (SEQ ID | H | NO: 8) | (SEQ ID |
| | | (SEQ ID | NO: 6) | (SEQ ID | | NO: 9) |
| | | NO: 35) | | NO: 7) | | |

TABLE 13
Heavy and Light Chain Variable Region Amino Acid Sequences
of Humanized 2A10 Variants

| | VH | VL |
|---|---|---|
| h2A10.v1 | EVQLVESGGGLVQPGPSLRLSCTAS | DIQLTQSPSSLSASVGDRVTITCRAS |
| | GFDFNSYGMSWVRQAPGKGLELV | QSVSISRFNLMHWYQHKPGKQPKL |
| | ADIVSKTYNYTTYYSDSVKDRFTIS | LIYRASNLASGIPSRFSGSGSGTDFT |
| | RDDSKSIVYLQMNSLKTEDTAVYY | LTISSLQPEDFATYYCQHSRESPWT |
| | CTVAPGGSFDYWGQGTLVTVSS | FGGGTKVEIK (SEQ ID NO: 36) |
| | (SEQ ID NO: 37) | |
| h2A10.v1.1 | Same as v1 (SEQ ID NO: 37) | DIQMTQSPSSLSASVGDRVTITCRA |
| | | SQSVSISRFNLMHWYQHKPGKQPK |
| | | LLIYRASNLASGIPSRFSGSGSGTDF |
| | | TLTISSLQPEDFATYYCQHSRESPW |
| | | TFGGGTKVEIK (SEQ ID NO: 38) |
| h2A10.v1.2 | Same as v1 (SEQ ID NO: 37) | DIQLTQSPSSLSASVGDRVTITCRAS |
| | | QSVSISRFNLMHWYQQKPGKQPKL |
| | | LIYRASNLASGIPSRFSGSGSGTDFT |
| | | LTISSLQPEDFATYYCQHSRESPWT |
| | | FGGGTKVEIK (SEQ ID NO: 39) |
| h2A10.v1.3 | Same as v1 (SEQ ID NO: 37) | DIQLTQSPSSLSASVGDRVTITCRAS |
| | | QSVSISRFNLMHWYQHKPGKAPKL |
| | | LIYRASNLASGIPSRFSGSGSGTDFT |
| | | LTISSLQPEDFATYYCQHSRESPWT |
| | | FGGGTKVEIK (SEQ ID NO: 40) |
| h2A10.v1.4 | Same as v1 (SEQ ID NO: 37) | DIQLTQSPSSLSASVGDRVTITCRAS |
| | | QSVSISRFNLMHWYQHKPGKQPKL |
| | | LIYRASNLASGVPSRFSGSGSGTDFT |
| | | LTISSLQPEDFATYYCQHSRESPWT |
| | | FGGGTKVEIK (SEQ ID NO: 41) |
| h2A10.v1.5 | EVQLVESGGGLVQPGPSLRLSCTAS | Same as v1 (SEQ ID NO: 36) |
| | GFDFNSYGMSWVRQAPGKGLEWV | |
| | ADIVSKTYNYTTYYSDSVKDRFTIS | |
| | RDDSKSIVYLQMNSLKTEDTAVYY | |
| | CTVAPGGSFDYWGQGTLVTVSS | |
| | (SEQ ID NO: 42) | |
| h2A10.v1.6 | EVQLVESGGGLVQPGPSLRLSCTAS | Same as v1 (SEQ ID NO: 36) |
| | GFDFNSYGMSWVRQAPGKGLELV | |
| | GDIVSKTYNYTTYYSDSVKDRFTIS | |
| | RDDSKSIVYLQMNSLKTEDTAVYY | |
| | CTVAPGGSFDYWGQGTLVTVSS | |
| | (SEQ ID NO: 43) | |
| h2A10.v1.7 | EVQLVESGGGLVQPGPSLRLSCTAS | Same as v1 (SEQ ID NO: 36) |
| | GFDFNSYGMSWVRQAPGKGLELV | |
| | ADIVSKTYNYTTYYSDSVKDRFTIS | |
| | RDDSKSIAYLQMNSLKTEDTAVYY | |
| | CTVAPGGSFDYWGQGTLVTVSS | |
| | (SEQ ID NO: 44) | |
| h2A10.v2 | EVQLLESGGGLVQPGGSLRLSCAAS | Same as v1 (SEQ ID NO: 36) |
| | GFDFNSYGMSWVRQAPGKGLELV | |
| | ADIVSKTYNYTTYYSDSVKDRFTIS | |
| | RDDSKSTVYLQMNSLRAEDTAVYY | |
| | CTVAPGGSFDYWGQGTLVTVSS | |
| | (SEQ ID NO: 45) | |
| h2A10.v2.1 | Same as v2 (SEQ ID NO: 45) | Same as v1.1 (SEQ ID NO: 38) |
| h2A10.v2.2 | Same as v2 (SEQ ID NO: 45) | Same as v1.2 (SEQ ID NO: 39) |
| h2A10.v2.3 | Same as v2 (SEQ ID NO: 45) | Same as v1.3 (SEQ ID NO: 40) |
| h2A10.v2.4 | Same as v2 (SEQ ID NO: 45) | Same as v1.4 (SEQ ID NO: 41) |
| h2A10.v2.5 | EVQLLESGGGLVQPGGSLRLSCAAS | Same as v1 (SEQ ID NO: 36) |
| | GFDFNSYGMSWVRQAPGKGLEWV | |
| | ADIVSKTYNYTTYYSDSVKDRFTIS | |
| | RDDSKSTVYLQMNSLRAEDTAVYY | |
| | CTVAPGGSFDYWGQGTLVTVSS | |
| | (SEQ ID NO: 46) | |
| h2A10.v2.6 | EVQLLESGGGLVQPGGSLRLSCAAS | Same as v1 (SEQ ID NO: 36) |
| | GFDFNSYGMSWVRQAPGKGLELVS | |
| | DIVSKTYNYTTYYSDSVKDRFTISR | |
| | DDSKSTVYLQMNSLRAEDTAVYYC | |
| | TVAPGGSFDYWGQGTLVTVSS | |
| | (SEQ ID NO: 47) | |
| h2A10.v2.7 | EVQLLESGGGLVQPGGSLRLSCAAS | Same as v1 (SEQ ID NO: 36) |
| | GFDFNSYGMSWVRQAPGKGLELV | |
| | ADIVSKTYNYTTYYSDSVKDRFTIS | |
| | RDNSKSTVYLQMNSLRAEDTAVYY | |
| | CTVAPGGSFDYWGQGTLVTVSS | |
| | (SEQ ID NO: 48) | |
| h2A10.v2.8 | EVQLLESGGGLVQPGGSLRLSCAAS | Same as v1 (SEQ ID NO: 36) |
| | GFDFNSYGMSWVRQAPGKGLELV | |
| | ADIVSKTYNYTTYYSDSVKDRFTIS | |
| | RDDSKNTVYLQMNSLRAEDTAVY | |
| | YCTVAPGGSFDYWGQGTLVTVSS | |
| | (SEQ ID NO: 49) | |
| h2A10.v2.9 | EVQLLESGGGLVQPGGSLRLSCAAS | Same as v1 (SEQ ID NO: 36) |
| | GFDFNSYGMSWVRQAPGKGLELV | |
| | ADIVSKTYNYTTYYSDSVKDRFTIS | |
| | RDDSKSTLYLQMNSLRAEDTAVYY | |
| | CTVAPGGSFDYWGQGTLVTVSS | |
| | (SEQ ID NO: 50) | |
| h2A10.v2.N54S | EVQLLESGGGLVQPGGSLRLSCAAS | Same as v1 (SEQ ID NO: 36) |
| | GFDFNSYGMSWVRQAPGKGLELV | |
| | ADIVSKTYSYTTYYSDSVKDRFTIS | |
| | RDDSKSTVYLQMNSLRAEDTAVYY | |
| | CTVAPGGSFDYWGQGTLVTVSS | |
| | (SEQ ID NO: 51) | |
| h2A10.v2.N54Q | EVQLLESGGGLVQPGGSLRLSCAAS | Same as v1 (SEQ ID NO: 36) |
| | GFDFNSYGMSWVRQAPGKGLELV | |
| | ADIVSKTYQYTTYYSDSVKDRFTIS | |
| | RDDSKSTVYLQMNSLRAEDTAVYY | |
| | CTVAPGGSFDYWGQGTLVTVSS | |
| | (SEQ ID NO: 52) | |
| h2A10.v2.T56A | EVQLLESGGGLVQPGGSLRLSCAAS | Same as v1 (SEQ ID NO: 36) |
| | GFDFNSYGMSWVRQAPGKGLELV | |
| | ADIVSKTYNYATYYSDSVKDRFTIS | |
| | RDDSKSTVYLQMNSLRAEDTAVYY | |
| | CTVAPGGSFDYWGQGTLVTVSS | |
| | (SEQ ID NO: 53) | |
| h2A10.v3 | EVQLLESGGGLVQPGGSLRLSCAAS | DIQLTQSPSSLSASVGDRVTITCRAS |
| | GFDFNSYGMSWVRQAPGKGLELVS | QSVSISRFNLMHWYQQKPGKAPKL |
| | DIVSKTYSYTTYYSDSVKDRFTISR | LIYRASNLASGVPSRFSGSGSGTDFT |
| | DDSKNTLYLQMNSLRAEDTAVYY | LTISSLQPEDFATYYCQHSRESPWT |
| | CTVAPGGSFDYWGQGTLVTVSS | FGGGTKVEIK (SEQ ID NO: 54) |
| | (SEQ ID NO: 55) | |
| h2A10.v4 | EVQLLESGGGLVQPGGSLRLSCAAS | DIQLTQSPSSLSASVGDRVTITCRAS |
| | GFDFNSYGMSWVRQAPGKGLELVS | QSVSISRFNLMHWYQQKPGKAPKL |
| | DIVSKTYNYATYYSDSVKDRFTISR | LIYRASNLASGVPSRFSGSGSGTDFT |
| | DDSKNTLYLQMNSLRAEDTAVYY | LTISSLQPEDFATYYCQHSRESPWT |
| | CTVAPGGSFDYWGQGTLVTVSS | FGGGTKVEIK (SEQ ID NO: 56) |
| | (SEQ ID NO: 57) | |

TABLE 14
Complete Heavy and Light Chain Sequences for Humanized 2A10 Variants

| | Complete H Chain | Complete L Chain |
|---|---|---|
| h2A10.v1 | EVQLVESGGGLVQPGPSLRLSCTASGFDF | DIQLTQSPSSLSASVGDRVTI |
| | NSYGMSWVRQAPGKGLELVADIVSKTYN | TCRASQSVSISRFNLMHWYQ |
| | YTTYYSDSVKDRFTISRDDSKSIVYLQMNS | HKPGKQPKLLIYRASNLASG |
| | LKTEDTAVYYCTVAPGGSFDYWGQGTLV | IPSRFSGSGSGTDFTLTISSLQ |
| | TVSSASTKGPSVFPLAPSSKSTSGGTAALG | PEDFATYYCQHSRESPWTFG |
| | CLVKDYFPEPVTVSWNSGALTSGVHTFPA | GGTKVEIKRTVAAPSVFIFPP |
| | VLQSSGLYSLSSVVTVPSSSLGTQTYICNV | SDEQLKSGTASVVCLLNNFY |
| | NHKPSNTKVDKKVEPKSCDKTHTCPPCPA | PREAKVQWKVDNALQSGNS |
| | PELLGGPSVFLFPPKPKDTLMISRTPEVTC | QESVTEQDSKDSTYSLSSTL |
| | VVVDVSHEDPEVKFNWYVDGVEVHNAK | TLSKADYEKHKVYACEVTH |
| | TKPREEQYGSTYRVVSVLTVLHQDWLNG | QGLSSPVTKSFNRGEC (SEQ |
| | KEYKCKVSNKALPAPIEKTISKAKGQPREP | ID NO: 58) |
| | QVYTLPPSREEMTKNQVSLTCLVKGFYPS | |
| | DIAVEWESNGQPENNYKTTPPVLDSDGSF | |
| | FLYSKLTVDKSRWQQGNVFSCSVMHEAL | |
| | HNHYTQKSLSLSPGK (SEQ ID NO: 59) | |
| h2A10.v1.1 | Same as v1 (SEQ ID NO: 59) | DIQMTQSPSSLSASVGDRVTI |
| | | TCRASQSVSISRFNLMHWYQ |
| | | HKPGKQPKLLIYRASNLASG |
| | | IPSRFSGSGSGTDFTLTISSLQ |
| | | PEDFATYYCQHSRESPWTFG |
| | | GGTKVEIKRTVAAPSVFIFPP |
| | | SDEQLKSGTASVVCLLNNFY |
| | | PREAKVQWKVDNALQSGNS |
| | | QESVTEQDSKDSTYSLSSTL |
| | | TLSKADYEKHKVYACEVTH |
| | | QGLSSPVTKSFNRGEC (SEQ |
| | | ID NO: 60) |
| h2A10.v1.2 | Same as v1 (SEQ ID NO: 59) | DIQLTQSPSSLSASVGDRVTI |
| | | TCRASQSVSISRFNLMHWYQ |
| | | QKPGKQPKLLIYRASNLASG |
| | | IPSRFSGSGSGTDFTLTISSLQ |
| | | PEDFATYYCQHSRESPWTFG |
| | | GGTKVEIKRTVAAPSVFIFPP |
| | | SDEQLKSGTASVVCLLNNFY |
| | | PREAKVQWKVDNALQSGNS |
| | | QESVTEQDSKDSTYSLSSTL |
| | | TLSKADYEKHKVYACEVTH |
| | | QGLSSPVTKSFNRGEC (SEQ |
| | | ID NO: 61) |
| h2A10.v1.3 | Same as v1 (SEQ ID NO: 59) | DIQLTQSPSSLSASVGDRVTI |
| | | TCRASQSVSISRFNLMHWYQ |
| | | HKPGKAPKLLIYRASNLASG |
| | | IPSRFSGSGSGTDFTLTISSLQ |
| | | PEDFATYYCQHSRESPWTFG |
| | | GGTKVEIKRTVAAPSVFIFPP |
| | | SDEQLKSGTASVVCLLNNFY |
| | | PREAKVQWKVDNALQSGNS |
| | | QESVTEQDSKDSTYSLSSTL |
| | | TLSKADYEKHKVYACEVTH |
| | | QGLSSPVTKSFNRGEC (SEQ |
| | | ID NO: 62) |
| h2A10.v1.4 | Same as v1 (SEQ ID NO: 59) | DIQLTQSPSSLSASVGDRVTI |
| | | TCRASQSVSISRFNLMHWYQ |
| | | HKPGKQPKLLIYRASNLASG |
| | | VPSRFSGSGSGTDFTLTISSL |
| | | QPEDFATYYCQHSRESPWTF |
| | | GGGTKVEIKRTVAAPSVFIFP |
| | | PSDEQLKSGTASVVCLLNNF |
| | | YPREAKVQWKVDNALQSG |
| | | NSQESVTEQDSKDSTYSLSS |
| | | TLTLSKADYEKHKVYACEV |
| | | THQGLSSPVTKSFNRGEC |
| | | (SEQ ID NO: 63) |
| h2A10.v1.5 | EVQLVESGGGLVQPGPSLRLSCTASGFDF | Same as v1 (SEQ ID NO: 58) |
| | NSYGMSWVRQAPGKGLEWVADIVSKTY | |
| | NYTTYYSDSVKDRFTISRDDSKSIVYLQM | |
| | NSLKTEDTAVYYCTVAPGGSFDYWGQGT | |
| | LVTVSSASTKGPSVFPLAPSSKSTSGGTAA | |
| | LGCLVKDYFPEPVTVSWNSGALTSGVHTF | |
| | PAVLQSSGLYSLSSVVTVPSSSLGTQTYIC | |
| | NVNHKPSNTKVDKKVEPKSCDKTHTCPPC | |
| | PAPELLGGPSVFLFPPKPKDTLMISRTPEVT | |
| | CWVDVSHEDPEVKFNWYVDGVEVHNA | |
| | KTKPREEQYGSTYRVVSVLTVLHQDWLN | |
| | GKEYKCKVSNKALPAPIEKTISKAKGQPR | |
| | EPQVYTLPPSREEMTKNQVSLTCLVKGFY | |
| | PSDIAVEWESNGQPENNYKTTPPVLDSDG | |
| | SFFLYSKLTVDKSRWQQGNVFSCSVMHE | |
| | ALHNHYTQKSLSLSPGK (SEQ ID NO: 64) | |
| h2A10.v1.6 | EVQLVESGGGLVQPGPSLRLSCTASGFDF | Same as v1 (SEQ ID NO: 58) |
| | NSYGMSWVRQAPGKGLELVGDIVSKTYN | |
| | YTTYYSDSVKDRFTISRDDSKSIVYLQMNS | |
| | LKTEDTAVYYCTVAPGGSFDYWGQGTLV | |
| | TVSSASTKGPSVFPLAPSSKSTSGGTAALG | |
| | CLVKDYFPEPVTVSWNSGALTSGVHTFPA | |
| | VLQSSGLYSLSSVVTVPSSSLGTQTYICNV | |
| | NHKPSNTKVDKKVEPKSCDKTHTCPPCPA | |
| | PELLGGPSVFLFPPKPKDTLMISRTPEVTC | |
| | VVVDVSHEDPEVKFNWYVDGVEVHNAK | |
| | TKPREEQYGSTYRVVSVLTVLHQDWLNG | |
| | KEYKCKVSNKALPAPIEKTISKAKGQPREP | |
| | QVYTLPPSREEMTKNQVSLTCLVKGFYPS | |
| | DIAVEWESNGQPENNYKTTPPVLDSDGSF | |
| | FLYSKLTVDKSRWQQGNVFSCSVMHEAL | |
| | HNHYTQKSLSLSPGK (SEQ ID NO: 65) | |
| h2A10.v1.7 | EVQLVESGGGLVQPGPSLRLSCTASGFDF | Same as v1 (SEQ ID NO: 58) |
| | NSYGMSWVRQAPGKGLELVADIVSKTYN | |
| | YTTYYSDSVKDRFTISRDDSKSIAYLQMNS | |
| | LKTEDTAVYYCTVAPGGSFDYWGQGTLV | |
| | TVSSASTKGPSVFPLAPSSKSTSGGTAALG | |
| | CLVKDYFPEPVTVSWNSGALTSGVHTFPA | |
| | VLQSSGLYSLSSVVTVPSSSLGTQTYICNV | |
| | NHKPSNTKVDKKVEPKSCDKTHTCPPCPA | |
| | PELLGGPSVFLFPPKPKDTLMISRTPEVTC | |
| | VVVDVSHEDPEVKFNWYVDGVEVHNAK | |
| | TKPREEQYGSTYRVVSVLTVLHQDWLNG | |
| | KEYKCKVSNKALPAPIEKTISKAKGQPREP | |
| | QVYTLPPSREEMTKNQVSLTCLVKGFYPS | |
| | DIAVEWESNGQPENNYKTTPPVLDSDGSF | |
| | FLYSKLTVDKSRWQQGNVFSCSVMHEAL | |
| | HNHYTQKSLSLSPGK (SEQ ID NO: 66) | |
| h2A10.v2 | EVQLLESGGGLVQPGGSLRLSCAASGFDF | Same as v1 (SEQ ID NO: 58) |
| | NSYGMSWVRQAPGKGLELVADIVSKTYN | |
| | YTTYYSDSVKDRFTISRDDSKSTVYLQMN | |
| | SLRAEDTAVYYCTVAPGGSFDYWGQGTL | |
| | VTVSSASTKGPSVFPLAPSSKSTSGGTAAL | |
| | GCLVKDYFPEPVTVSWNSGALTSGVHTFP | |
| | AVLQSSGLYSLSSVVTVPSSSLGTQTYICN | |
| | VNHKPSNTKVDKKVEPKSCDKTHTCPPCP | |
| | APELLGGPSVFLFPPKPKDTLMISRTPEVT | |
| | CVVVDVSHEDPEVKFNWYVDGVEVHNA | |
| | KTKPREEQYGSTYRVVSVLTVLHQDWLN | |
| | GKEYKCKVSNKALPAPIEKTISKAKGQPR | |
| | EPQVYTLPPSREEMTKNQVSLTCLVKGFY | |
| | PSDIAVEWESNGQPENNYKTTPPVLDSDG | |
| | SFFLYSKLTVDKSRWQQGNVFSCSVMHE | |
| | ALHNHYTQKSLSLSPGK (SEQ ID NO: 67) | |
| h2A10.v2.1 | Same as v2 (SEQ ID NO: 67) | Same as v1.1 (SEQ ID NO: 60) |
| h2A10.v2.2 | Same as v2 (SEQ ID NO: 67) | Same as v1.2 (SEQ ID NO: 61) |
| h2A10.v2.3 | Same as v2 (SEQ ID NO: 67) | Same as v1.3 (SEQ ID NO: 62) |
| h2A10.v2.4 | Same as v2 (SEQ ID NO: 67) | Same as v1.4 (SEQ ID NO: 63) |
| h2A10.v2.5 | EVQLLESGGGLVQPGGSLRLSCAASGFDF | Same as v1 (SEQ ID NO: 58) |
| | NSYGMSWVRQAPGKGLEWVADIVSKTY | |
| | NYTTYYSDSVKDRFTISRDDSKSTVYLQM | |
| | NSLRAEDTAVYYCTVAPGGSFDYWGQGT | |
| | LVTVSSASTKGPSVFPLAPSSKSTSGGTAA | |
| | LGCLVKDYFPEPVTVSWNSGALTSGVHTF | |
| | PAVLQSSGLYSLSSVVTVPSSSLGTQTYIC | |
| | NVNHKPSNTKVDKKVEPKSCDKTHTCPPC | |
| | PAPELLGGPSVFLFPPKPKDTLMISRTPEVT | |
| | CWVDVSHEDPEVKFNWYVDGVEVHNA | |
| | KTKPREEQYGSTYRVVSVLTVLHQDWLN | |
| | GKEYKCKVSNKALPAPIEKTISKAKGQPR | |
| | EPQVYTLPPSREEMTKNQVSLTCLVKGFY | |
| | PSDIAVEWESNGQPENNYKTTPPVLDSDG | |
| | SFFLYSKLTVDKSRWQQGNVFSCSVMHE | |
| | ALHNHYTQKSLSLSPGK (SEQ ID NO: 68) | |
| h2A10.v2.6 | EVQLLESGGGLVQPGGSLRLSCAASGFDF | Same as v1 (SEQ ID NO: 58) |
| | NSYGMSWVRQAPGKGLELVSDIVSKTYN | |
| | YTTYYSDSVKDRFTISRDDSKSTVYLQMN | |
| | SLRAEDTAVYYCTVAPGGSFDYWGQGTL | |
| | VTVSSASTKGPSVFPLAPSSKSTSGGTAAL | |
| | GCLVKDYFPEPVTVSWNSGALTSGVHTFP | |
| | AVLQSSGLYSLSSVVTVPSSSLGTQTYICN | |
| | VNHKPSNTKVDKKVEPKSCDKTHTCPPCP | |
| | APELLGGPSVFLFPPKPKDTLMISRTPEVT | |
| | CWVDVSHEDPEVKFNWYVDGVEVHNA | |
| | KTKPREEQYGSTYRVVSVLTVLHQDWLN | |
| | GKEYKCKVSNKALPAPIEKTISKAKGQPR | |
| | EPQVYTLPPSREEMTKNQVSLTCLVKGFY | |
| | PSDIAVEWESNGQPENNYKTTPPVLDSDG | |
| | SFFLYSKLTVDKSRWQQGNVFSCSVMHE | |
| | ALHNHYTQKSLSLSPGK (SEQ ID NO: 69) | |
| h2A10.v2.7 | EVQLLESGGGLVQPGGSLRLSCAASGFDF | Same as v1 (SEQ ID NO: 58) |
| | NSYGMSWVRQAPGKGLELVADIVSKTYN | |
| | YTTYYSDSVKDRFTISRDNSKSTVYLQMN | |
| | SLRAEDTAVYYCTVAPGGSFDYWGQGTL | |
| | VTVSSASTKGPSVFPLAPSSKSTSGGTAAL | |
| | GCLVKDYFPEPVTVSWNSGALTSGVHTFP | |
| | AVLQSSGLYSLSSVVTVPSSSLGTQTYICN | |
| | VNHKPSNTKVDKKVEPKSCDKTHTCPPCP | |
| | APELLGGPSVFLFPPKPKDTLMISRTPEVT | |
| | CWVDVSHEDPEVKFNWYVDGVEVHNA | |
| | KTKPREEQYGSTYRVVSVLTVLHQDWLN | |
| | GKEYKCKVSNKALPAPIEKTISKAKGQPR | |
| | EPQVYTLPPSREEMTKNQVSLTCLVKGFY | |
| | PSDIAVEWESNGQPENNYKTTPPVLDSDG | |
| | SFFLYSKLTVDKSRWQQGNVFSCSVMHE | |
| | ALHNHYTQKSLSLSPGK (SEQ ID NO: 70) | |
| h2A10.v2.8 | EVQLLESGGGLVQPGGSLRLSCAASGFDF | Same as v1 (SEQ ID NO: 58) |
| | NSYGMSWVRQAPGKGLELVADIVSKTYN | |
| | YTTYYSDSVKDRFTISRDDSKNTVYLQMN | |
| | SLRAEDTAVYYCTVAPGGSFDYWGQGTL | |
| | VTVSSASTKGPSVFPLAPSSKSTSGGTAAL | |
| | GCLVKDYFPEPVTVSWNSGALTSGVHTFP | |
| | AVLQSSGLYSLSSVVTVPSSSLGTQTYICN | |
| | VNHKPSNTKVDKKVEPKSCDKTHTCPPCP | |
| | APELLGGPSVFLFPPKPKDTLMISRTPEVT | |
| | CWVDVSHEDPEVKFNWYVDGVEVHNA | |
| | KTKPREEQYGSTYRVVSVLTVLHQDWLN | |
| | GKEYKCKVSNKALPAPIEKTISKAKGQPR | |
| | EPQVYTLPPSREEMTKNQVSLTCLVKGFY | |
| | PSDIAVEWESNGQPENNYKTTPPVLDSDG | |
| | SFFLYSKLTVDKSRWQQGNVFSCSVMHE | |
| | ALHNHYTQKSLSLSPGK (SEQ ID NO: 71) | |
| h2A10.v2.9 | EVQLLESGGGLVQPGGSLRLSCAASGFDF | Same as v1 (SEQ ID NO: 58) |
| | NSYGMSWVRQAPGKGLELVADIVSKTYN | |
| | YTTYYSDSVKDRFTISRDDSKSTLYLQMN | |
| | SLRAEDTAVYYCTVAPGGSFDYWGQGTL | |
| | VTVSSASTKGPSVFPLAPSSKSTSGGTAAL | |
| | GCLVKDYFPEPVTVSWNSGALTSGVHTFP | |
| | AVLQSSGLYSLSSVVTVPSSSLGTQTYICN | |
| | VNHKPSNTKVDKKVEPKSCDKTHTCPPCP | |
| | APELLGGPSVFLFPPKPKDTLMISRTPEVT | |
| | CWVDVSHEDPEVKFNWYVDGVEVHNA | |
| | KTKPREEQYGSTYRVVSVLTVLHQDWLN | |
| | GKEYKCKVSNKALPAPIEKTISKAKGQPR | |
| | EPQVYTLPPSREEMTKNQVSLTCLVKGFY | |
| | PSDIAVEWESNGQPENNYKTTPPVLDSDG | |
| | SFFLYSKLTVDKSRWQQGNVFSCSVMHE | |
| | ALHNHYTQKSLSLSPGK (SEQ ID NO: 72) | |
| h2A10.v2.N54S | EVQLLESGGGLVQPGGSLRLSCAASGFDF | Same as v1 (SEQ ID NO: 58) |
| | NSYGMSWVRQAPGKGLELVADIVSKTYS | |
| | YTTYYSDSVKDRFTISRDDSKSTVYLQMN | |
| | SLRAEDTAVYYCTVAPGGSFDYWGQGTL | |
| | VTVSSASTKGPSVFPLAPSSKSTSGGTAAL | |
| | GCLVKDYFPEPVTVSWNSGALTSGVHTFP | |
| | AVLQSSGLYSLSSVVTVPSSSLGTQTYICN | |
| | VNHKPSNTKVDKKVEPKSCDKTHTCPPCP | |
| | APELLGGPSVFLFPPKPKDTLMISRTPEVT | |
| | CWVDVSHEDPEVKFNWYVDGVEVHNA | |
| | KTKPREEQYGSTYRVVSVLTVLHQDWLN | |
| | GKEYKCKVSNKALPAPIEKTISKAKGQPR | |
| | EPQVYTLPPSREEMTKNQVSLTCLVKGFY | |
| | PSDIAVEWESNGQPENNYKTTPPVLDSDG | |
| | SFFLYSKLTVDKSRWQQGNVFSCSVMHE | |
| | ALHNHYTQKSLSLSPGK (SEQ ID NO: 73) | |
| h2A10.v2.N54Q | EVQLLESGGGLVQPGGSLRLSCAASGFDF | Same as v1 (SEQ ID NO: 58) |
| | NSYGMSWVRQAPGKGLELVADIVSKTYQ | |
| | YTTYYSDSVKDRFTISRDDSKSTVYLQMN | |
| | SLRAEDTAVYYCTVAPGGSFDYWGQGTL | |
| | VTVSSASTKGPSVFPLAPSSKSTSGGTAAL | |
| | GCLVKDYFPEPVTVSWNSGALTSGVHTFP | |
| | AVLQSSGLYSLSSVVTVPSSSLGTQTYICN | |
| | VNHKPSNTKVDKKVEPKSCDKTHTCPPCP | |
| | APELLGGPSVFLFPPKPKDTLMISRTPEVT | |
| | CWVDVSHEDPEVKFNWYVDGVEVHNA | |
| | KTKPREEQYGSTYRVVSVLTVLHQDWLN | |
| | GKEYKCKVSNKALPAPIEKTISKAKGQPR | |
| | EPQVYTLPPSREEMTKNQVSLTCLVKGFY | |
| | PSDIAVEWESNGQPENNYKTTPPVLDSDG | |
| | SFFLYSKLTVDKSRWQQGNVFSCSVMHE | |
| | ALHNHYTQKSLSLSPGK (SEQ ID NO: 74) | |
| h2A10.v2.T56A | EVQLLESGGGLVQPGGSLRLSCAASGFDF | Same as v1 (SEQ ID NO: 58) |
| | NSYGMSWVRQAPGKGLELVADIVSKTYN | |
| | YATYYSDSVKDRFTISRDDSKSTVYLQMN | |
| | SLRAEDTAVYYCTVAPGGSFDYWGQGTL | |
| | VTVSSASTKGPSVFPLAPSSKSTSGGTAAL | |
| | GCLVKDYFPEPVTVSWNSGALTSGVHTFP | |
| | AVLQSSGLYSLSSVVTVPSSSLGTQTYICN | |
| | VNHKPSNTKVDKKVEPKSCDKTHTCPPCP | |
| | APELLGGPSVFLFPPKPKDTLMISRTPEVT | |
| | CWVDVSHEDPEVKFNWYVDGVEVHNA | |
| | KTKPREEQYGSTYRVVSVLTVLHQDWLN | |
| | GKEYKCKVSNKALPAPIEKTISKAKGQPR | |
| | EPQVYTLPPSREEMTKNQVSLTCLVKGFY | |
| | PSDIAVEWESNGQPENNYKTTPPVLDSDG | |
| | SFFLYSKLTVDKSRWQQGNVFSCSVMHE | |
| | ALHNHYTQKSLSLSPGK (SEQ ID NO: 75) | |
| h2A10.v3 | EVQLLESGGGLVQPGGSLRLSCAASGFDF | DIQLTQSPSSLSASVGDRVTI |
| | NSYGMSWVRQAPGKGLELVSDIVSKTYS | TCRASQSVSISRFNLMHWYQ |
| | YTTYYSDSVKDRFTISRDDSKNTLYLQMN | QKPGKAPKLLIYRASNLASG |
| | SLRAEDTAVYYCTVAPGGSFDYWGQGTL | VPSRFSGSGSGTDFTLTISSL |
| | VTVSSASTKGPSVFPLAPSSKSTSGGTAAL | QPEDFATYYCQHSRESPWTF |
| | GCLVKDYFPEPVTVSWNSGALTSGVHTFP | GGGTKVEIKRTVAAPSVFIFP |
| | AVLQSSGLYSLSSVVTVPSSSLGTQTYICN | PSDEQLKSGTASVVCLLNNF |
| | VNHKPSNTKVDKKVEPKSCDKTHTCPPCP | YPREAKVQWKVDNALQSG |
| | APELLGGPSVFLFPPKPKDTLMISRTPEVT | NSQESVTEQDSKDSTYSLSS |
| | CWVDVSHEDPEVKFNWYVDGVEVHNA | TLTLSKADYEKHKVYACEV |
| | KTKPREEQYGSTYRVVSVLTVLHQDWLN | THQGLSSPVTKSFNRGEC |
| | GKEYKCKVSNKALPAPIEKTISKAKGQPR | (SEQ ID NO: 76) |
| | EPQVYTLPPSREEMTKNQVSLTCLVKGFY | |
| | PSDIAVEWESNGQPENNYKTTPPVLDSDG | |
| | SFFLYSKLTVDKSRWQQGNVFSCSVMHE | |
| | ALHNHYTQKSLSLSPGK (SEQ ID NO: 77) | |
| h2A10.v4 | EVQLLESGGGLVQPGGSLRLSCAASGFDF | DIQLTQSPSSLSASVGDRVTI |
| | NSYGMSWVRQAPGKGLELVSDIVSKTYN | TCRASQSVSISRFNLMHWYQ |
| | YATYYSDSVKDRFTISRDDSKNTLYLQMN | QKPGKAPKLLIYRASNLASG |
| | SLRAEDTAVYYCTVAPGGSFDYWGQGTL | VPSRFSGSGSGTDFTLTISSL |
| | VTVSSASTKGPSVFPLAPSSKSTSGGTAAL | QPEDFATYYCQHSRESPWTF |
| | GCLVKDYFPEPVTVSWNSGALTSGVHTFP | GGGTKVEIKRTVAAPSVFIFP |
| | AVLQSSGLYSLSSVVTVPSSSLGTQTYICN | PSDEQLKSGTASVVCLLNNF |
| | VNHKPSNTKVDKKVEPKSCDKTHTCPPCP | YPREAKVQWKVDNALQSG |
| | APELLGGPSVFLFPPKPKDTLMISRTPEVT | NSQESVTEQDSKDSTYSLSS |
| | CVVVDVSHEDPEVKFNWYVDGVEVHNA | TLTLSKADYEKHKVYACEV |
| | KTKPREEQYGSTYRVVSVLTVLHQDWLN | THQGLSSPVTKSFNRGEC |
| | GKEYKCKVSNKALPAPIEKTISKAKGQPR | (SEQ ID NO: 78) |
| | EPQVYTLPPSREEMTKNQVSLTCLVKGFY | |
| | PSDIAVEWESNGQPENNYKTTPPVLDSDG | |
| | SFFLYSKLTVDKSRWQQGNVFSCSVMHE | |
| | ALHNHYTQKSLSLSPGK (SEQ ID NO: 79) | |

### Example 7: Cynomolgus Monkey PK Study of 2A10 Humanized Variants

### Materials and Methods

Eight experimentally naïve female cynomolgus monkeys (Macaca fascicularis, Cambodian origin) were assigned to one of two groups (n=4/group). At the outset (Day −1) of the study, animals were approximately 4 years old and weighed approximately 2.5-4 kg, and only animals that appeared to be healthy and that were free of obvious abnormalities were used for the study. Animals in Groups 1 or 2 were given a single 10 mg/kg I.V. bolus dose of anti-TGFβ3 h2A10.v3 or h2A10.v4, respectively. Doses were administered via the cephalic vein in the forearm. At various timepoints up to 43 days post-dose, serum samples (n=4/timepoint) were collected and analyzed for serum test article concentrations by ELISA methods.

The serum concentrations of animals dosed with 10 mg/kg of anti-TGFβ3 variants h2A10.v3 and h2A10.v4 were determined by an ELISA method. 384-well plates were coated with 0.5 g/mL sheep anti-human IgG antibody (Binding Site, San Diego, CA, USA) diluted in coat buffer (0.05 M carbonate/bicarbonate buffer pH 9.6) and incubated overnight at 4 °C. The plates were washed 3 times with wash buffer (0.5% Tween-20 in PBS buffer, pH 7.4) and treated with block buffer (PBS/0.5% BSA/15 ppm Proclin, pH 7.4) for 1 to 2 hours at room temperature (RT). The plates were again washed 3 times with wash buffer and then samples diluted in sample diluent (PBS/0.5% BSA/0.05% Tween 20/5 mM EDTA/0.25% CHAPS/0.35M NaCl/15 ppm Proclin, pH 7.4) were added to the wells and incubated for 2 hours at RT. The next day, the plates were washed 6 times with wash buffer. A detection antibody, goat anti-human IgG (H+L)-horseradish peroxidase (HRP) (Bethyl Laboratories, Inc., Montgomery, TX, USA), diluted to 100 ng/mL in assay buffer (PBS/0.5% BSA/15 ppm Proclin/0.05% Tween 20, pH7.4) was added to the wells and incubated on a shaker for 1 hour at RT. The plates were washed 6 times with wash buffer and developed using TMB peroxidase substrate (Moss Inc., Pasadena, Maryland) for 20 minutes followed by 1 M Phosphoric acid to stop the reaction. Absorbance was measured at 450 nm against a reference wavelength of 620 nm. The concentration of the samples was extrapolated from a 4-parameter fit of the standard curve. The reportable assay range was 0.156-10 ng/mL. The minimum required dilution was 1:100 with the Minimum Quantifiable Concentration at 16 ng/mL.

### Results:

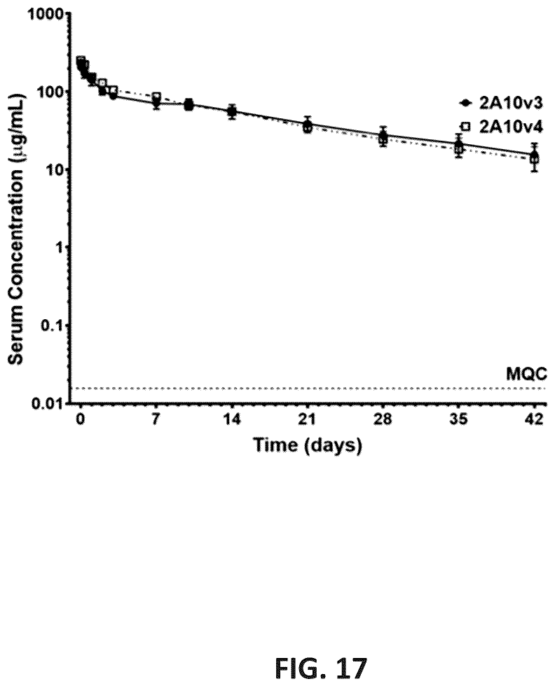

Following a single I.V. bolus dose of anti-TGFβ3 h2A10.v3 at 10 mg/kg, mean clearance (CL) was 4.35 mL/day/kg, Cₘₐₓ was 238 g/mL, exposure (AUCinf) was 2400 day·μg/mL, terminal half-life (t_{1/2}, λz) was 15.1 days, volume of distribution at steady state (Vₛₛ) was 88.2 mL/kg. h2A10.v3 demonstrated apparent biphasic distribution characterized by a rapid initial distribution phase followed by a slower elimination phase (FIG. 17).

Following a single I.V. bolus dose of anti-TGFβ3 h2A10.v4 at 10 mg/kg, mean clearance (CL) was 4.21 mL/day/kg, Cₘₐₓ was 252 μg/mL, exposure (AUCinf) was 2470 day·μg/mL, terminal half-life (t_{1/2}, λz) was 16.0 days, volume of distribution at steady state (Vₛₛ) was 79.1 mL/kg, respectively. 2A10.v4 demonstrated apparent biphasic distribution characterized by a rapid initial distribution phase followed by a slower elimination phase.

The pharmacokinetics of anti-TGFβ3 variants h2A10.v3 and h2A10.v4 following a single I.V. bolus administration at 10 mg/kg was as expected for a human IgG1 monoclonal antibody in cynomolgus monkeys and similar to that of typical IgG1 and IgG4 monoclonal antibodies. The PK data are summarized in Table 15, below.

TABLE 15
Results of cyno PK study

| PK Parameter | 2A10.v3; | 2A10.v4 |
|---|---|---|
| | 10 mg/kg IV; (n = 4) | 10 mg/kg IV; (n = 4) |
| Cₘₐₓ (μg/mL) | 238 ± 2.26 | 252 ± 6.96 |
| AUCinf (day · μg/mL) | 2400 ± 539 | 2470 ± 559 |
| CL (mL/kg/day) | 4.35 ± 1.05 | 4.21 ± 0.952 |
| t_{1/2}, λz (day) | 15.1 ± 2.26 | 16.0 ± 5.63 |
| V_{SS} (mL/kg) | 88.2 ± 6.40 | 79.1 ± 9.63 |

### Example 8: Humanization of 4A11 (Dual-Specific Anti-TGFβ2/3 Antibody)

### Materials and Methods

### Antibody Affinity Measurement by BIAcore

Binding affinity determinations of all humanized 4A11 antibodies were performed using SPR measurement with a BIAcore™-T200 instrument, as described above in Example 6 (2A11 Ab example).

### HEKBlue Cell-Based TGFβ Blocking Assay

HEKBlue cell-based TGFβ3 blocking assay was performed as described above in Example 6.

### Results:

### Humanization of 4A11:

4A11 Humanization Step 1: Grafting Rabbit 4A11 CDRs onto Human Frameworks:

Rabbit B-cell derived monoclonal antibody 4A11 was humanized as described below. Residue numbers are according to Kabat et al., Sequences of proteins of immunological interest, 5th Ed., Public Health Service, National Institutes of Health, Bethesda, MD (1991).

The variable regions of the rabbit 4A11 (Rbt 4A11) antibody were first aligned with all human germlines. Four light chain human germlines (IGKV4-1, IGKV1D-39, IGKV3-20, IGKV2-24) and two heavy chain human germlines (IGHV3-48, IGHV4-59) were identified as the closest acceptor frameworks for grafting Rbt 4A11 light chain and heavy chain hypervariable regions (CDRs) respectively. Specifically, from Rbt 4A11 light chain variable domain (VL), positions 24-34 (L1), 50-56 (L2) and 89-97 (L3) were grafted into IGKV4-1, IGKV1D-39, IGKV3-20 and IGKV2-24. From Rbt 4A11 heavy chain variable domain (VH), positions 26-35 (H1), 50-65 (H2) and 95-102 (H3) were grafted into IGHV3-48 and IGHV4-59 respectively.

Eight humanized variants were generated by shuffling different grafting acceptor frameworks: Humanized 4A11 version 1 (h4A11.v1) is by IGKV4-1 and IGHV3-48; humanized 4A11 version 2 (h4A11.v2) is by IGKV1D-39 and IGHV3-48; humanized 4A11 version 3 (h4A11.v3) is by IGKV4-1 and IGHV4-59; humanized 4A11 version 4 (h4A11.v4) is by IGKV1D-39 and IGHV4-59; humanized 4A11 version 5 (h4A11.v5) is by IGKV3-20 and IGHV3-48; humanized 4A11 version 6 (h4A11.v6) is by IGKV2-24 and IGHV3-48; humanized 4A11 version 7 (h4A11.v7) is by IGKV3-20 and IGHV4-59; humanized 4A11 version 8 (h4A11.v8) is by IGKV2-24 and IGHV4-59 (Table 16).

In addition to CDRs grafted onto the closest acceptor frameworks described above, further modifications were made as follows:

### Light Chain Framework:

h4A11.v1 and h4A11.v3 used the same IGKV4-1 acceptor framework, retained rabbit residues at position 2 and 4 in framework I and position 36 in framework II of VL from Rbt 4A11.
h4A11.v2 and h4A11.v4 used the same IGKV1D-39 acceptor framework, retained rabbit residues at position 2 and 4 in framework I and position 36 and 43 in framework II of VL from Rbt 4A11.
h4A11.v5 and h4A11.v7 used the same IGKV3-20 acceptor framework, retained rabbit residues at position 2 in framework I, position 36 and 43 in framework II and position 58 in framework III of VL from Rbt 4A11.
h4A11.v6 and h4A11.v8, used the same IGKV2-24 acceptor framework, retained rabbit residues at position 2 and 4 in framework I and position 36 in framework II of VL from Rbt 4A11.

### Heavy Chain Framework:

h4A11.v1, h4A11.v2, h4A11.v5, h4A11.v6, used the same IGHV3-48 acceptor framework, retained rabbit residues at position 2 and 24 in framework I, position 48 and 49 in framework II, position 71, 73, 78 and 91 in framework III and position 105 in framework IV of VH from Rbt 4A11.
h4A11.v3, h4A11.v4, h4A11.v7, h4A11.v8, used the same IGHV4-59 acceptor framework, retained rabbit residues at position 2 in framework I, position 37 in framework II, position 67, 71, 73, 78 and 91 in framework III and position 105 in framework IV of VH from Rbt 4A11.

All of these residues were found to be the part of framework residues acting as “Vernier” zone, which may adjust CDR structure and fine-tune the antigen fit. See, e.g., Foote and Winter, J. Mol. Biol. 224: 487-499 (1992). These CDR definitions include positions defined by their sequence hypervariability (Wu, T. T. & Kabat, E. A. (1970)), their structural location (Chothia, C. & Lesk, A. M. (1987)) and their involvement in antigen-antibody contacts (MacCallum et al. J. Mol. Biol. 262: 732-745 (1996)).

All eight humanized 4A11 variants were further subjected to BIAcore for human TGFβ isoforms binding affinity determination and HEKBlue cell-based assay for human TGFβ isoforms blocking potency assessment. Although there was some drop (3-10 fold) in human TGFβ2&3 binding for most of the variants, the degree of drop in human TGFβ2&3 blocking were more pronounced and significant (Table 16). In all of the eight humanized versions, only h4A11.v7 retained comparable human TGFβ2&3 blocking compared to Rbt 4A11 despite a few fold drop in human TGFβ2&3 binding. Therefore, this clone was moved forward for the next step of humanization, framework polishing.

TABLE 16
4A11 humanization step 1 variants characterization summary

| 4A11 | | | | | | | |
|---|---|---|---|---|---|---|---|
| variants | Human | BIAcore (pM) | | | HEKBlue TGFP blocking IC50 (nM) | | |
| humanized | Framework | TGFβ1 | TGFβ2 | TGFβ3 | TGFβ1 | TGFβ2 | TGFβ3 |
| Rbt 4A11 | — | >5000 | 1.6 | ≤1.2 | >200 | 0.09 | 0.06 |
| h4A11.v1 | K₄₋₁H₃₋₄₈ | >5000 | 4.6 | 14.4 | >200 | >200 | >200 |
| h4A11.v2 | K_{1D-39}H₃₋₄₈ | >5000 | 1.4 | 13.3 | >200 | >200 | >200 |
| h4A11.v3 | K₄₋₁H₄₋₅₉ | >5000 | 9.6 | 12.1 | >200 | 3.9 | 0.23 |
| h4A11.v4 | K_{1D-39}H₄₋₅₉ | >5000 | 10.0 | 10.1 | >200 | 1.9 | 0.08 |
| h4A11.v5 | K₃₋₂₀H₃₋₄₈ | >5000 | 12.1 | 13.9 | >200 | >200 | 1.4 |
| h4A11.v6 | K₂₋₂₄H₃₋₄₈ | >5000 | 8.4 | 12.5 | >200 | >200 | 6.7 |
| h4A11.v7 | K₃₋₂₀H₄₋₅₉ | >5000 | 6.3 | 9.7 | >200 | 0.09 | 0.03 |
| h4A11.v8 | K₂₋₂₄H₄₋₅₉ | >5000 | 8.2 | 14.1 | >200 | >200 | 0.66 |

### 4A11 Humanization Step 2: H4A11.v7 Framework Polishing:

In framework polishing of h4A11.v7, three variants (v7.1-v7.3) were generated to delete the amino acid insertion being introduced from the step 1 humanization. Next, other humanized variants were generated by substituting each rabbit residue of h4A11.v7 at Vernier zone with the corresponding human germline residues (v7.4-v7.15). To determine which residues were necessary to be retained, binding to TGFβ isoforms of each variant was then evaluated by BIAcore and blocking potency was assessed in HEKBlue cell-based assay.

In h4A11.v7, these framework polishing variants are h4A11.v7.1 (VH: Delete E1), h4A11.v7.2 (VH: Delete K75 and N76), h4A11.v7.3 (VH: Delete E1, K75 and N76), h4A11.v7.4 (VL: A2I), h4A11.v7.5 (VL: F36Y), h4A11.v7.6 (VL: P43A), h4A11.v7.7 (VL: V58I), h4A11.v7.8 (VH: Q2V), h4A11.v7.9 (VH: V37I), h4A11.v7.10 (VH: F67V), h4A11.v7.11 (VH: K71V), h4A11.v7.12 (VH: S73T), h4A11.v7.13 (VH: V78F), h4A11.v7.14 (VH: F91Y), h4A11.v7.15 (VH: P105Q) (Table 17).

Most of the variants appeared to be similar or slightly improved in comparison with parental h4A11.v7 in binding and potency assay, except for the major impact on h4A11.v7.11 (VH: K71V) with some drop in TGFβ2 binding (6-fold) and TGFβ3 binding (2-fold), and significantly, more loss in TGFβ2 blocking (>2000-fold) and TGFβ3 blocking (118-fold). There was one variant with minor impact: h4A11.v7.5 (VL: F36Y) with near 2-fold drop in both TGFβ2 binding and blocking, and two variants with sub-minor impact: h4A11.v7.13 (VH: V78F) with 2.5-fold drop in TGFβ2 binding; h4A11.v7.14 (VH: F91Y) with 4-fold drop in TGFβ2 blocking (Table 17). In summary, it was determined that all rabbit residues at Vernier zone of h4A11.v7 could be replaced with human germline residues, except for major position 71 in framework III of VH, minor position 36 in framework II of VL and/or sub-minor position 78 and 91 in framework III of VH.

TABLE 17
4A11 humanization step 2 variants characterization summary

| | | | | | | HEKBlue TGFβ3 | | |
|---|---|---|---|---|---|---|---|---|
| h4A11.v7 | Human | Framework | BIAcore (pM) | | | blocking IC50 (nM) | | |
| variants | Framework | mutation | TGFβ1 | TGFβ2 | TGFβ3 | TGFβ1 | TGFβ2 | TGFβ3 |
| Rbt 4A11 | | — | >5000 | 1.6 | ≤1.2 | >200 | 0.09 | 0.06 |
| h4A11.v7 | K₃₋₂₀H₄₋₅₉ | — | >5000 | 6.3 | 9.7 | >200 | 0.09 | 0.03 |
| h4A11.v7.1 | K₃₋₂₀H₄₋₅₉ | HC: Delete E1 | na | na | na | >33 | 0.09 | 0.03 |
| h4A11.v7.2 | K₃₋₂₀H₄₋₅₉ | HC: Delete | na | na | na | >33 | 0.03 | 0.01 |
| | | K75 & N76 | | | | | | |
| h4A11.v7.3 | K₃₋₂0H₄₋₅₉ | HC: Delete | na | na | na | >33 | 0.03 | 0.03 |
| | | E1, K75 & N76 | | | | | | |
| h4A11.v7.4 | K₃₋₂₀H₄₋₅₉ | LC: A2I | >5000 | 6.4 | 4.3 | >200 | 0.01 | 0.05 |
| h4A11.v7.5 | K₃₋₂₀H₄₋₅₉ | LC: F36Y | >5000 | 9.2 | 9.3 | >200 | 0.16 | 0.04 |
| h4A11.v7.6 | K₃₋₂₀H₄₋₅₉ | LC: P43A | >5000 | 5.8 | 6.5 | >200 | 0.01 | 0.01 |
| h4A11.v7.7 | K₃₋₂₀H₄₋₅₉ | LC: V58I | >5000 | 6.3 | 6.1 | >200 | 0.04 | 0.04 |
| h4A11.v7.8 | K₃₋₂₀H₄₋₅₉ | HC: Q2V | >5000 | 4.5 | 5.3 | >200 | 0.06 | 0.03 |
| h4A11.v7.9 | K₃₋₂₀H₄₋₅₉ | HC: V37I | >5000 | 4.9 | 5.7 | >200 | 0.08 | 0.02 |
| h4A11.v7.10 | K₃₋₂₀H₄₋₅₉ | HC: F67V | >5000 | 5.6 | 6.8 | >200 | 0.02 | 0.02 |
| h4A11.v7.11 | K₃₋₂₀H₄₋₅₉ | HC: K71V | >5000 | 40.1 | 21.4 | >200 | >200 | 3.53 |
| h4A11.v7.12 | K₃₋₂₀H₄₋₅₉ | HC: S73T | >5000 | 6.0 | 6.5 | >200 | 0.01 | 0.03 |
| h4A11.v7.13 | K₃₋₂₀H₄₋₅₉ | HC: V78F | >5000 | 15.9 | 8.9 | >200 | 0.03 | 0.05 |
| h4A11.v7.14 | K₃₋₂₀H₄₋₅₉ | HC: F91Y | >5000 | 4.9 | 5.5 | >200 | 0.35 | 0.04 |
| h4A11.v7.15 | K₃₋₂₀H₄₋₅₉ | HC: P105Q | >5000 | 4.7 | 4.8 | >200 | 0.09 | 0.02 |

4A11 humanization step 3: Final version generation and selection According to the results from h4A11.v7 framework polishing, h4A11.v7.18 was first generated by replacing h4A11.v7 non-crucial framework residues at Vernier zone with the corresponding human germline residues, which were A to I at position 2 in framework, P to A at position 43 in framework II and V to I at position 58 in framework III of VL, and Q to V at position 2 in framework I, V to I at position 37 in framework II, F to V at position 67 in framework III, S to T at position 73 in framework III, P to Q at position 105 in framework IV of VH (Table 18). Together with all the above substitutions, two other sub-minor framework residues in h4A11.v7, V78 or F91 in framework III of VH, were also replaced with the corresponding human germline residues to generate the other 2 humanized versions, h4A11.v7.16 (VH: V78F) and h4A11.v7.17 (VH: 1F91Y). One more humanized version, h4A11.v7.19, was generated by incorporating the deletion of K75 and N76 in framework III of VH, which was beneficial in improving TGFβ2 & 3 blocking in the framework polishing of (h4A11.v7 (Table 17, above: h4A11.v7.2 and h4A11.v7.3).

By comparison of all 4 final humanized versions, interestingly, only h4A11.v7.19 appeared to be superior than h4A11.v7 and comparable to parental Rbt 4A11 not only in human TGFβ32 and TGFβ33 binding, but also in human TGFβ32 and TGFβ33 blocking.

TABLE 18
4A11 humanization step 3 variants characterization summary

| | | | | | | HEKBlue TGFβ3 | | |
|---|---|---|---|---|---|---|---|---|
| h4A11.v7 | Human | Framework | BIAcore (pM) | | | blocking IC50 (nM) | | |
| variants | Framework | mutation | TGFβ1 | TGFβ2 | TGFβ3 | TGFβ1 | TGFβ2 | TGFβ3 |
| Rbt 4A11 | | — | >5000 | 1.6 | ≤1.2 | >200 | 0.09 | 0.06 |
| h4A11.v7 | K₃₋₂₀H₄₋₅₉ | — | >5000 | 6.3 | 9.7 | >200 | 0.09 | 0.03 |
| h4A11.v7.16 | K₃₋₂₀H₄₋₅₉ | LC: A2I, P43A, V58I | >5000 | 4.9 | 6.6 | >200 | 0.09 | 0.13 |
| | | HC: Q2V, V37I, F67V, | | | | | | |
| | | S73T, V78F, P105Q | | | | | | |
| h4A11.v7.17 | K₃₋₂₀H₄₋₅₉ | LC:A2I, P43A, V58I | >5000 | ≤1.3 | 3.2 | >200 | 0.16 | 0.17 |
| | | HC: Q2V, V371, F67V, | | | | | | |
| | | S73T, F91Y, P105Q | | | | | | |
| h4A11.v7.18 | K₃₋₂₀H₄₋₅₉ | LC:A2I, P43A, V58I | >5000 | ≤1.1 | 4.6 | >200 | 0.09 | 0.11 |
| | | HC: Q2V, V37I, F67V, | | | | | | |
| | | S73T, P105Q | | | | | | |
| h4A11.v7.19 | K₃₋₂₀H₄₋₅₉ | LC: A2I, P43A, V58I | >5000 | ≤1.0 | ≤1.3 | >200 | 0.05 | 0.09 |
| | | HC: Q2V, V37I, F67V, | | | | | | |
| | | S73T, Delete K⁷⁵N⁷⁶, | | | | | | |
| | | P105Q | | | | | | |

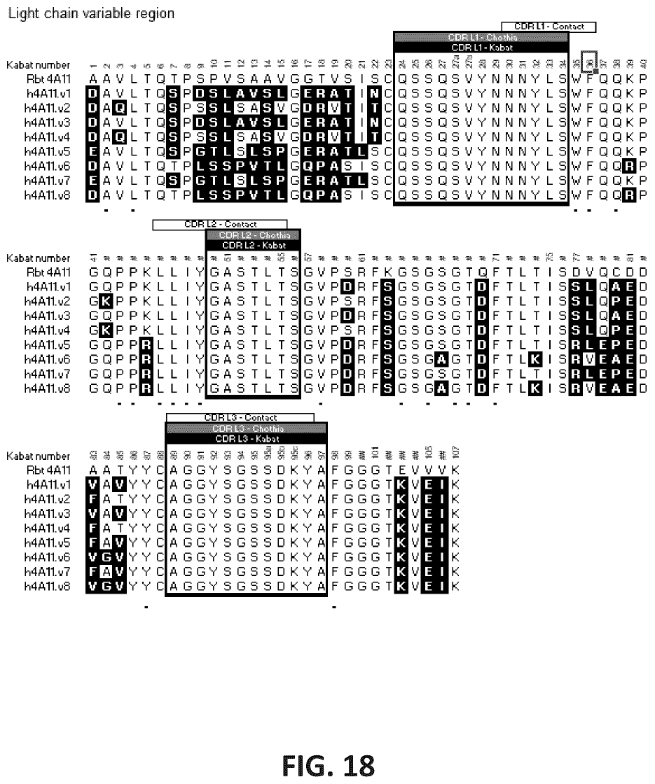

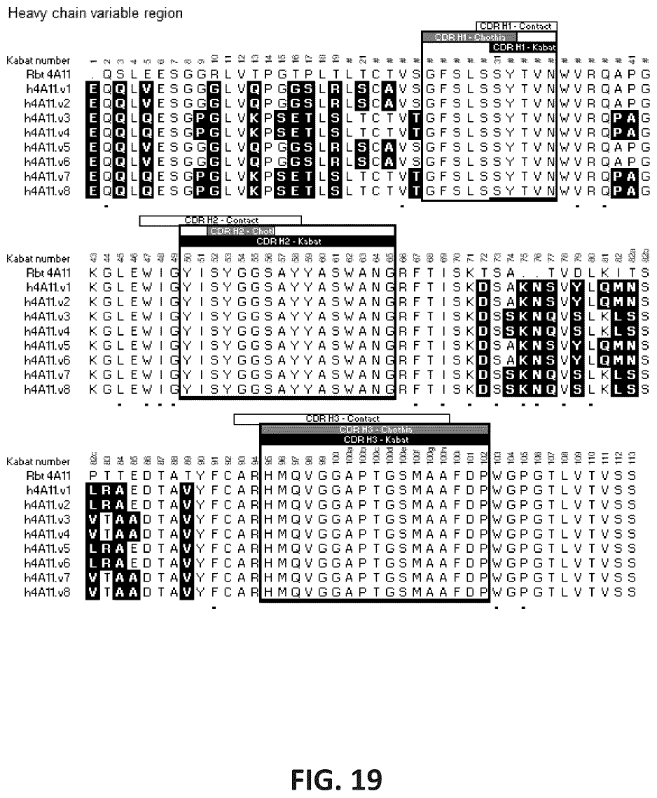

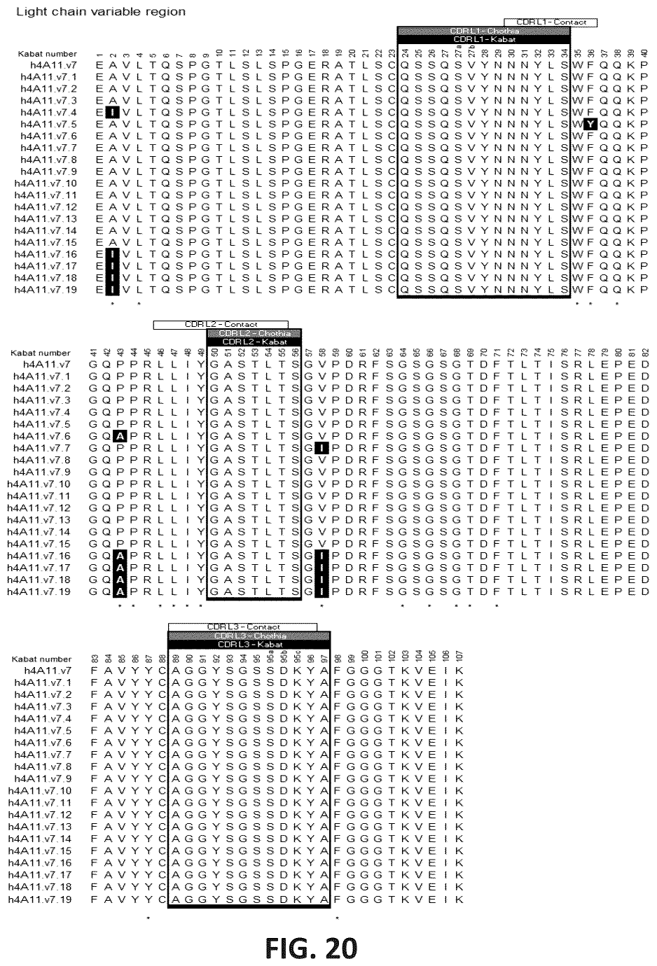

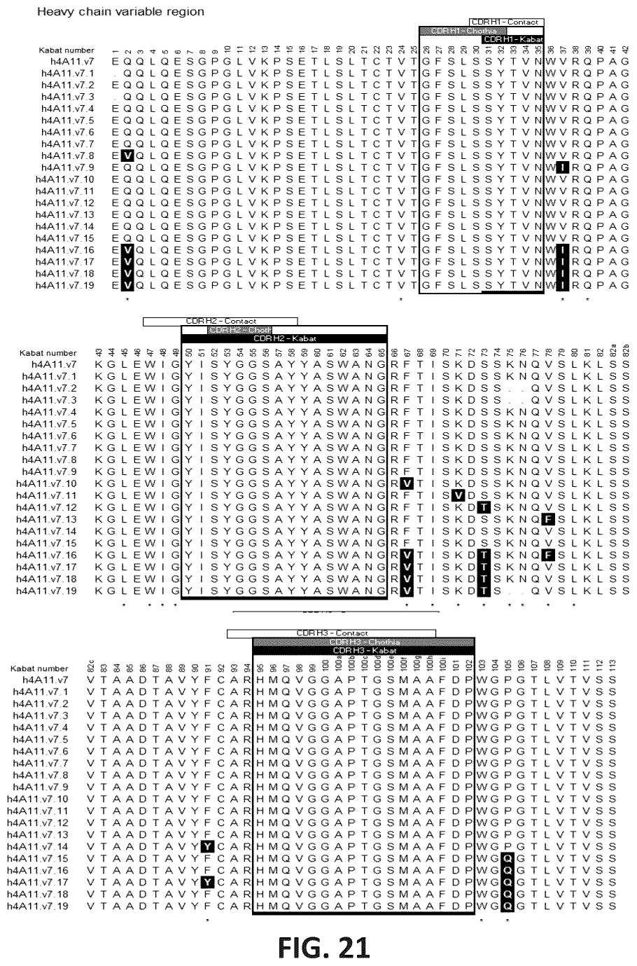

The amino acids sequences of the 4A11 variants are provided in Table 19 and Table 20, below, and sequence alignments are shown in FIG. 18, FIG. 19, FIG. 20, and FIG. 21.

TABLE 19
Heavy and Light Chain Variable Region Amino Acid Sequences of
Humanized 4A11 Variants

| | VH | VL |
|---|---|---|
| h4A11.v1 | EQQLVESGGGLVQPGGSLRLSCAVS | DAVLTQSPDSLAVSLGERATINCQ |
| | GFSLSSYTVNWVRQAPGKGLEWIG | SSQSVYNNNYLSWFQQKPGQPPK |
| | YISYGGSAYYASWANGRFTISKDSA | LLIYGASTLTSGVPDRFSGSGSGT |
| | KNSVYLQMNSLRAEDTAVYFCARH | DFTLTISSLQAEDVAVYYCAGGY |
| | MQVGGAPTGSMAAFDPWGPGTLVT | SGSSDKYAFGGGTKVEIK (SEQ ID |
| | VSS (SEQ ID NO: 81) | NO: 80) |
| h4A11.v2 | Same as v1 (SEQ ID NO: 81) | DAQLTQSPSSLSASVGDRVTITCQ |
| | | SSQSVYNNNYLSWFQQKPGKPPK |
| | | LLIYGASTLTSGVPSRFSGSGSGT |
| | | DFTLTISSLQPEDFATYYCAGGYS |
| | | GSSDKYAFGGGTKVEIK (SEQ ID |
| | | NO: 82) |
| h4A11.v3 | EQQLQESGPGLVKPSETLSLTCTVTG | Same as v1 (SEQ ID NO: 80) |
| | FSLSSYTVNWVRQPAGKGLEWIGYI | |
| | SYGGSAYYASWANGRFTISKDSSKN | |
| | QVSLKLSSVTAADTAVYFCARHMQ | |
| | VGGAPTGSMAAFDPWGPGTLVTVS | |
| | S (SEQ ID NO: 83) | |
| h4A11.v4 | Same as v3 (SEQ ID NO: 83) | Same as v2 (SEQ ID NO: 82) |
| h4A11.v5 | Same as v1 (SEQ ID NO: 81) | EAVLTQSPGTLSLSPGERATLSCQ |
| | | SSQSVYNNNYLSWFQQKPGQPPR |
| | | LLIYGASTLTSGVPDRFSGSGSGT |
| | | DFTLTISRLEPEDFAVYYCAGGYS |
| | | GSSDKYAFGGGTKVEIK (SEQ ID |
| | | NO: 84) |
| h4A11.v6 | Same as v1 (SEQ ID NO: 81) | DAVLTQTPLSSPVTLGQPASISCQ |
| | | SSQSVYNNNYLSWFQQKPGQPPR |
| | | LLIYGASTLTSGVPDRFSGSGAGT |
| | | DFTLKISRVEAEDVGVYYCAGGY |
| | | SGSSDKYAFGGGTKVEIK (SEQ ID |
| | | NO: 85) |
| h4A11.v7 | Same as V3 (SEQ ID NO: 83) | Same as v5 (SEQ ID NO: 84) |
| h4A11.v7. | QQLQESGPGLVKPSETLSLTCTVTGF | Same as v5 (SEQ ID NO: 84) |
| 1 | SLSSYTVNWVRQPAGKGLEWIGYIS | |
| | YGGSAYYASWANGRFTISKDSSKNQ | |
| | VSLKLSSVTAADTAVYFCARHMQV | |
| | GGAPTGSMAAFDPWGPGTLVTVSS | |
| | (SEQ ID NO: 86) | |
| h4A11.v7. | EQQLQESGPGLVKPSETLSLTCTVTG | Same as v5 (SEQ ID NO: 84) |
| 2 | FSLSSYTVNWVRQPAGKGLEWIGYI | |
| | SYGGSAYYASWANGRFTISKDSSQV | |
| | SLKLSSVTAADTAVYFCARHMQVG | |
| | GAPTGSMAAFDPWGPGTLVTVSS | |
| | (SEQ ID NO: 87) | |
| h4A11.v7. | QQLQESGPGLVKPSETLSLTCTVTGF | Same as v5 (SEQ ID NO: 84) |
| 3 | SLSSYTVNWVRQPAGKGLEWIGYIS | |
| | YGGSAYYASWANGRFTISKDSSQVS | |
| | LKLSSVTAADTAVYFCARHMQVGG | |
| | APTGSMAAFDPWGPGTLVTVSS | |
| | (SEQ ID NO: 88) | |
| h4A11.v7. | Same as v3 (SEQ ID NO: 83) | EIVLTQSPGTLSLSPGERATLSCQS |
| 4 | | SQSVYNNNYLSWFQQKPGQPPRL |
| | | LIYGASTLTSGVPDRFSGSGSGTD |
| | | FTLTISRLEPEDFAVYYCAGGYSG |
| | | SSDKYAFGGGTKVEIK (SEQ ID |
| | | NO: 89) |
| h4A11.v7. | Same as v3 (SEQ ID NO: 83) | EAVLTQSPGTLSLSPGERATLSCQ |
| 5 | | SSQSVYNNNYLSWYQQKPGQPPR |
| | | LLIYGASTLTSGVPDRFSGSGSGT |
| | | DFTLTISRLEPEDFAVYYCAGGYS |
| | | GSSDKYAFGGGTKVEIK (SEQ ID |
| | | NO: 90) |
| h4A11.v7. | Same as v3 (SEQ ID NO: 83) | EAVLTQSPGTLSLSPGERATLSCQ |
| 6 | | SSQSVYNNNYLSWFQQKPGQAPR |
| | | LLIYGASTLTSGVPDRFSGSGSGT |
| | | DFTLTISRLEPEDFAVYYCAGGYS |
| | | GSSDKYAFGGGTKVEIK (SEQ ID |
| | | NO: 91) |
| h4A11.v7. | Same as v3 (SEQ ID NO: 83) | EAVLTQSPGTLSLSPGERATLSCQ |
| 7 | | SSQSVYNNNYLSWFQQKPGQPPR |
| | | LLIYGASTLTSGIPDRFSGSGSGTD |
| | | FTLTISRLEPEDFAVYYCAGGYSG |
| | | SSDKYAFGGGTKVEIK (SEQ ID |
| | | NO: 92) |
| h4A11.v7. | EVQLQESGPGLVKPSETLSLTCTVTG | Same as v5 (SEQ ID NO: 84) |
| 8 | FSLSSYTVNWVRQPAGKGLEWIGYI | |
| | SYGGSAYYASWANGRFTISKDSSKN | |
| | QVSLKLSSVTAADTAVYFCARHMQ | |
| | VGGAPTGSMAAFDPWGPGTLVTVS | |
| | S (SEQ ID NO: 93) | |
| h4A11.v7. | EQQLQESGPGLVKPSETLSLTCTVTG | Same as v5 (SEQ ID NO: 84) |
| 9 | FSLSSYTVNWIRQPAGKGLEWIGYIS | |
| | YGGSAYYASWANGRFTISKDSSKNQ | |
| | VSLKLSSVTAADTAVYFCARHMQV | |
| | GGAPTGSMAAFDPWGPGTLVTVSS | |
| | (SEQ ID NO: 94) | |
| h4A11.v7. | EQQLQESGPGLVKPSETLSLTCTVTG | Same as v5 (SEQ ID NO: 84) |
| 10 | FSLSSYTVNWVRQPAGKGLEWIGYI | |
| | SYGGSAYYASWANGRVTISKDSSKN | |
| | QVSLKLSSVTAADTAVYFCARHMQ | |
| | VGGAPTGSMAAFDPWGPGTLVTVS | |
| | S (SEQ ID NO: 95) | |
| h4A11.v7. | EQQLQESGPGLVKPSETLSLTCTVTG | Same as v5 (SEQ ID NO: 84) |
| 11 | FSLSSYTVNWVRQPAGKGLEWIGYI | |
| | SYGGSAYYASWANGRFTISVDSSKN | |
| | QVSLKLSSVTAADTAVYFCARHMQ | |
| | VGGAPTGSMAAFDPWGPGTLVTVS | |
| | S (SEQ ID NO: 96) | |
| h4A11.v7. | EQQLQESGPGLVKPSETLSLTCTVTG | Same as v5 (SEQ ID NO: 84) |
| 12 | FSLSSYTVNWVRQPAGKGLEWIGYI | |
| | SYGGSAYYASWANGRFTISKDTSKN | |
| | QVSLKLSSVTAADTAVYFCARHMQ | |
| | VGGAPTGSMAAFDPWGPGTLVTVS | |
| | S (SEQ ID NO: 97) | |
| h4A11.v7. | EQQLQESGPGLVKPSETLSLTCTVTG | Same as v5 (SEQ ID NO: 84) |
| 13 | FSLSSYTVNWVRQPAGKGLEWIGYI | |
| | SYGGSAYYASWANGRFTISKDSSKN | |
| | QFSLKLSSVTAADTAVYFCARHMQ | |
| | VGGAPTGSMAAFDPWGPGTLVTVS | |
| | S (SEQ ID NO: 98) | |
| h4A11.v7. | EQQLQESGPGLVKPSETLSLTCTVTG | Same as v5 (SEQ ID NO: 84) |
| 14 | FSLSSYTVNWVRQPAGKGLEWIGYI | |
| | SYGGSAYYASWANGRFTISKDSSKN | |
| | QVSLKLSSVTAADTAVYYCARHMQ | |
| | VGGAPTGSMAAFDPWGPGTLVTVS | |
| | S (SEQ ID NO: 99) | |
| h4A11.v7. | EQQLQESGPGLVKPSETLSLTCTVTG | Same as v5 (SEQ ID NO: 84) |
| 15 | FSLSSYTVNWVRQPAGKGLEWIGYI | |
| | SYGGSAYYASWANGRFTISKDSSKN | |
| | QVSLKLSSVTAADTAVYFCARHMQ | |
| | VGGAPTGSMAAFDPWGQGTLVTVS | |
| | S (SEQ ID NO: 100) | |
| h4A11.v7. | EVQLQESGPGLVKPSETLSLTCTVTG | EIVLTQSPGTLSLSPGERATLSCQS |
| 16 | FSLSSYTVNWIRQPAGKGLEWIGYIS | SQSVYNNNYLSWFQQKPGQAPRL |
| | YGGSAYYASWANGRVTISKDTSKN | LIYGASTLTSGIPDRFSGSGSGTDF |
| | QFSLKLSSVTAADTAVYFCARHMQ | TLTISRLEPEDFAVYYCAGGYSGS |
| | VGGAPTGSMAAFDPWGQGTLVTVS | SDKYAFGGGTKVEIK (SEQ ID |
| | S (SEQ ID NO: 102) | NO: 101) |
| h4A11.v7. | EVQLQESGPGLVKPSETLSLTCTVTG | Same as v7.16 |
| 17 | FSLSSYTVNWIRQPAGKGLEWIGYIS | (SEQ ID NO: 101) |
| | YGGSAYYASWANGRVTISKDTSKN | |
| | QVSLKLSSVTAADTAVYYCARHMQ | |
| | VGGAPTGSMAAFDPWGQGTLVTVS | |
| | S (SEQ ID NO: 103) | |
| h4A11.v7. | EVQLQESGPGLVKPSETLSLTCTVTG | Same as v7.16 |
| 18 | FSLSSYTVNWIRQPAGKGLEWIGYIS | (SEQ ID NO: 101) |
| | YGGSAYYASWANGRVTISKDTSKN | |
| | QVSLKLSSVTAADTAVYFCARHMQ | |
| | VGGAPTGSMAAFDPWGQGTLVTVS | |
| | S (SEQ ID NO: 104) | |
| h4A11.v7. | EVQLQESGPGLVKPSETLSLTCTVTG | Same as v7.16 |
| 19 | FSLSSYTVNWIRQPAGKGLEWIGYIS | (SEQ ID NO: 101) |
| | YGGSAYYASWANGRVTISKDTSQV | |
| | SLKLSSVTAADTAVYFCARHMQVG | |
| | GAPTGSMAAFDPWGQGTLVTVSS | |
| | (SEQ ID NO: 105) | |
| h4A11.v8 | Same as v3 (SEQ ID NO: 83) | Same as v6 (SEQ ID NO: 85) |

TABLE 20
Complete Heavy and Light Chain Sequences of Humanized 4A11 Variants

| | Complete H Chain | Complete L Chain |
|---|---|---|
| h4A11. | EQQLVESGGGLVQPGGSLRLSCAVSGFSLSS | DAVLTQSPDSLAVSLGERAT |
| v1 | YTVNWVRQAPGKGLEWIGYISYGGSAYYAS | INCQSSQSVYNNNYLSWFQQ |
| | WANGRFTISKDSAKNSVYLQMNSLRAEDTA | KPGQPPKLLIYGASTLTSGVP |
| | VYFCARHMQVGGAPTGSMAAFDPWGPGTL | DRFSGSGSGTDFTLTISSLQA |
| | VTVSSASTKGPSVFPLAPSSKSTSGGTAALGC | EDVAVYYCAGGYSGSSDKY |
| | LVKDYFPEPVTVSWNSGALTSGVHTFPAVL | AFGGGTKVEIKRTVAAPSVFI |
| | QSSGLYSLSSVVTVPSSSLGTQTYICNVNHKP | FPPSDEQLKSGTASVVCLLN |
| | SNTKVDKKVEPKSCDKTHTCPPCPAPELLGG | NFYPREAKVQWKVDNALQS |
| | PSVFLFPPKPKDTLMISRTPEVTCVVVDVSHE | GNSQESVTEQDSKDSTYSLS |
| | DPEVKFNWYVDGVEVHNAKTKPREEQYGS | STLTLSKADYEKHKVYACE |
| | TYRVVSVLTVLHQDWLNGKEYKCKVSNKA | VTHQGLSSPVTKSFNRGEC |
| | LPAPIEKTISKAKGQPREPQVYTLPPSREEMT | (SEQ ID NO: 106) |
| | KNQVSLTCLVKGFYPSDIAVEWESNGQPEN | |
| | NYKTTPPVLDSDGSFFLYSKLTVDKSRWQQ | |
| | GNVFSCSVMHEALHNHYTQKSLSLSPGK | |
| | (SEQ ID NO: 107) | |
| h4A11. | Same as v1 (SEQ ID NO: 107) | DAQLTQSPSSLSASVGDRVTI |
| v2 | | TCQSSQSVYNNNYLSWFQQ |
| | | KPGKPPKLLIYGASTLTSGVP |
| | | SRFSGSGSGTDFTLTISSLQPE |
| | | DFATYYCAGGYSGSSDKYA |
| | | FGGGTKVEIKRTVAAPSVFIF |
| | | PPSDEQLKSGTASVVCLLNN |
| | | FYPREAKVQWKVDNALQSG |
| | | NSQESVTEQDSKDSTYSLSST |
| | | LTLSKADYEKHKVYACEVT |
| | | HQGLSSPVTKSFNRGEC |
| | | (SEQ ID NO: 108) |
| h4A11. | EQQLQESGPGLVKPSETLSLTCTVTGFSLSSY | Same as v1 (SEQ ID NO: 106) |
| v3 | TVNWVRQPAGKGLEWIGYISYGGSAYYAS | |
| | WANGRFTISKDSSKNQVSLKLSSVTAADTAV | |
| | YFCARHMQVGGAPTGSMAAFDPWGPGTLV | |
| | TVSSASTKGPSVFPLAPSSKSTSGGTAALGCL | |
| | VKDYFPEPVTVSWNSGALTSGVHTFPAVLQS | |
| | SGLYSLSSVVTVPSSSLGTQTYICNVNHKPSN | |
| | TKVDKKVEPKSCDKTHTCPPCPAPELLGGPS | |
| | VFLFPPKPKDTLMISRTPEVTCVVVDVSHED | |
| | PEVKFNWYVDGVEVHNAKTKPREEQYGST | |
| | YRVVSVLTVLHQDWLNGKEYKCKVSNKAL | |
| | PAPIEKTISKAKGQPREPQVYTLPPSREEMTK | |
| | NQVSLTCLVKGFYPSDIAVEWESNGQPENN | |
| | YKTTPPVLDSDGSFFLYSKLTVDKSRWQQG | |
| | NVFSCSVMHEALHNHYTQKSLSLSPGK (SEQ | |
| | ID NO: 109) | |
| h4A11. | Same as v3 (SEQ ID NO: 109) | Same as v2 (SEQ ID NO: 108) |
| v4 | | |
| h4A11. | Same as v1 (SEQ ID NO: 107) | EAVLTQSPGTLSLSPGERATL |
| v5 | | SCQSSQSVYNNNYLSWFQQ |
| | | KPGQPPRLLIYGASTLTSGVP |
| | | DRFSGSGSGTDFTLTISRLEP |
| | | EDFAVYYCAGGYSGSSDKY |
| | | AFGGGTKVEIKRTVAAPSVFI |
| | | FPPSDEQLKSGTASVVCLLN |
| | | NFYPREAKVQWKVDNALQS |
| | | GNSQESVTEQDSKDSTYSLS |
| | | STLTLSKADYEKHKVYACE |
| | | VTHQGLSSPVTKSFNRGEC |
| | | (SEQ ID NO: 110) |
| h4A11. | Same as v1 (SEQ ID NO: 107) | DAVLTQTPLSSPVTLGQPASI |
| v6 | | SCQSSQSVYNNNYLSWFQQ |
| | | RPGQPPRLLIYGASTLTSGVP |
| | | DRFSGSGAGTDFTLKISRVE |
| | | AEDVGVYYCAGGYSGSSDK |
| | | YAFGGGTKVEIKRTVAAPSV |
| | | FIFPPSDEQLKSGTASVVCLL |
| | | NNFYPREAKVQWKVDNALQ |
| | | SGNSQESVTEQDSKDSTYSL |
| | | SSTLTLSKADYEKHKVYACE |
| | | VTHQGLSSPVTKSFNRGEC |
| | | (SEQ ID NO: 111) |
| h4A11. | Same as v3 (SEQ ID NO: 109) | Same as v5 (SEQ ID NO: 110) |
| v7 | | |
| h4A11. | QQLQESGPGLVKPSETLSLTCTVTGFSLSSYT | Same as v5 (SEQ ID NO: 110) |
| v7.1 | VNWVRQPAGKGLEWIGYISYGGSAYYASW | |
| | ANGRFTISKDSSKNQVSLKLSSVTAADTAVY | |
| | FCARHMQVGGAPTGSMAAFDPWGPGTLVT | |
| | VSSASTKGPSVFPLAPSSKSTSGGTAALGCLV | |
| | KDYFPEPVTVSWNSGALTSGVHTFPAVLQSS | |
| | GLYSLSSVVTVPSSSLGTQTYICNVNHKPSNT | |
| | KVDKKVEPKSCDKTHTCPPCPAPELLGGPSV | |
| | FLFPPKPKDTLMISRTPEVTCVVVDVSHEDPE | |
| | VKFNWYVDGVEVHNAKTKPREEQYGSTYR | |
| | VVSVLTVLHQDWLNGKEYKCKVSNKALPA | |
| | PIEKTISKAKGQPREPQVYTLPPSREEMTKNQ | |
| | VSLTCLVKGFYPSDIAVEWESNGQPENNYKT | |
| | TPPVLDSDGSFFLYSKLTVDKSRWQQGNVFS | |
| | CSVMHEALHNHYTQKSLSLSPGK (SEQ ID | |
| | NO: 112) | |
| h4A11. | EQQLQESGPGLVKPSETLSLTCTVTGFSLSSY | Same as v5 (SEQ ID NO: 110) |
| v7.2 | TVNWVRQPAGKGLEWIGYISYGGSAYYAS | |
| | WANGRFTISKDSSQVSLKLSSVTAADTAVYF | |
| | CARHMQVGGAPTGSMAAFDPWGPGTLVTV | |
| | SSASTKGPSVFPLAPSSKSTSGGTAALGCLVK | |
| | DYFPEPVTVSWNSGALTSGVHTFPAVLQSSG | |
| | LYSLSSVVTVPSSSLGTQTYICNVNHKPSNTK | |
| | VDKKVEPKSCDKTHTCPPCPAPELLGGPSVF | |
| | LFPPKPKDTLMISRTPEVTCVVVDVSHEDPE | |
| | VKFNWYVDGVEVHNAKTKPREEQYGSTYR | |
| | VVSVLTVLHQDWLNGKEYKCKVSNKALPA | |
| | PIEKTISKAKGQPREPQVYTLPPSREEMTKNQ | |
| | VSLTCLVKGFYPSDIAVEWESNGQPENNYKT | |
| | TPPVLDSDGSFFLYSKLTVDKSRWQQGNVFS | |
| | CSVMHEALHNHYTQKSLSLSPGK (SEQ ID | |
| | NO: 113) | |
| h4A11. | QQLQESGPGLVKPSETLSLTCTVTGFSLSSYT | Same as v5 (SEQ ID NO: 110) |
| v7.3 | VNWVRQPAGKGLEWIGYISYGGSAYYASW | |
| | ANGRFTISKDSSQVSLKLSSVTAADTAVYFC | |
| | ARHMQVGGAPTGSMAAFDPWGPGTLVTVS | |
| | SASTKGPSVFPLAPSSKSTSGGTAALGCLVK | |
| | DYFPEPVTVSWNSGALTSGVHTFPAVLQSSG | |
| | LYSLSSVVTVPSSSLGTQTYICNVNHKPSNTK | |
| | VDKKVEPKSCDKTHTCPPCPAPELLGGPSVF | |
| | LFPPKPKDTLMISRTPEVTCVVVDVSHEDPE | |
| | VKFNWYVDGVEVHNAKTKPREEQYGSTYR | |
| | VVSVLTVLHQDWLNGKEYKCKVSNKALPA | |
| | PIEKTISKAKGQPREPQVYTLPPSREEMTKNQ | |
| | VSLTCLVKGFYPSDIAVEWESNGQPENNYKT | |
| | TPPVLDSDGSFFLYSKLTVDKSRWQQGNVFS | |
| | CSVMHEALHNHYTQKSLSLSPGK (SEQ ID | |
| | NO: 114) | |
| h4A11. | Same as v7.3 (SEQ ID NO: 114) | EIVLTQSPGTLSLSPGERATL |
| v7.4 | | SCQSSQSVYNNNYLSWFQQ |
| | | KPGQPPRLLIYGASTLTSGVP |
| | | DRFSGSGSGTDFTLTISRLEP |
| | | EDFAVYYCAGGYSGSSDKY |
| | | AFGGGTKVEIKRTVAAPSVFI |
| | | FPPSDEQLKSGTASVVCLLN |
| | | NFYPREAKVQWKVDNALQS |
| | | GNSQESVTEQDSKDSTYSLS |
| | | STLTLSKADYEKHKVYACE |
| | | VTHQGLSSPVTKSFNRGEC |
| | | (SEQ ID NO: 115) |
| h4A11. | Same as v7.3 (SEQ ID NO: 114) | EAVLTQSPGTLSLSPGERATL |
| v7.5 | | SCQSSQSVYNNNYLSWYQQ |
| | | KPGQPPRLLIYGASTLTSGVP |
| | | DRFSGSGSGTDFTLTISRLEP |
| | | EDFAVYYCAGGYSGSSDKY |
| | | AFGGGTKVEIKRTVAAPSVFI |
| | | FPPSDEQLKSGTASVVCLLN |
| | | NFYPREAKVQWKVDNALQS |
| | | GNSQESVTEQDSKDSTYSLS |
| | | STLTLSKADYEKHKVYACE |
| | | VTHQGLSSPVTKSFNRGEC |
| | | (SEQ ID NO: 116) |
| h4A11. | Same as v7.3 (SEQ ID NO: 114) | EAVLTQSPGTLSLSPGERATL |
| v7.6 | | SCQSSQSVYNNNYLSWFQQ |
| | | KPGQAPRLLIYGASTLTSGVP |
| | | DRFSGSGSGTDFTLTISRLEP |
| | | EDFAVYYCAGGYSGSSDKY |
| | | AFGGGTKVEIKRTVAAPSVFI |
| | | FPPSDEQLKSGTASVVCLLN |
| | | NFYPREAKVQWKVDNALQS |
| | | GNSQESVTEQDSKDSTYSLS |
| | | STLTLSKADYEKHKVYACE |
| | | VTHQGLSSPVTKSFNRGEC |
| | | (SEQ ID NO: 117) |
| h4A11. | Same as v7.3 (SEQ ID NO: 114) | EAVLTQSPGTLSLSPGERATL |
| v7.7 | | SCQSSQSVYNNNYLSWFQQ |
| | | KPGQPPRLLIYGASTLTSGIP |
| | | DRFSGSGSGTDFTLTISRLEP |
| | | EDFAVYYCAGGYSGSSDKY |
| | | AFGGGTKVEIKRTVAAPSVFI |
| | | FPPSDEQLKSGTASVVCLLN |
| | | NFYPREAKVQWKVDNALQS |
| | | GNSQESVTEQDSKDSTYSLS |
| | | STLTLSKADYEKHKVYACE |
| | | VTHQGLSSPVTKSFNRGEC |
| | | (SEQ ID NO: 118) |
| h4A11. | EVQLQESGPGLVKPSETLSLTCTVTGFSLSSY | Same as v5 (SEQ ID NO: 110) |
| v7.8 | TVNWVRQPAGKGLEWIGYISYGGSAYYAS | |
| | WANGRFTISKDSSKNQVSLKLSSVTAADTAV | |
| | YFCARHMQVGGAPTGSMAAFDPWGPGTLV | |
| | TVSSASTKGPSVFPLAPSSKSTSGGTAALGCL | |
| | VKDYFPEPVTVSWNSGALTSGVHTFPAVLQS | |
| | SGLYSLSSVVTVPSSSLGTQTYICNVNHKPSN | |
| | TKVDKKVEPKSCDKTHTCPPCPAPELLGGPS | |
| | VFLFPPKPKDTLMISRTPEVTCVVVDVSHED | |
| | PEVKFNWYVDGVEVHNAKTKPREEQYGST | |
| | YRVVSVLTVLHQDWLNGKEYKCKVSNKAL | |
| | PAPIEKTISKAKGQPREPQVYTLPPSREEMTK | |
| | NQVSLTCLVKGFYPSDIAVEWESNGQPENN | |
| | YKTTPPVLDSDGSFFLYSKLTVDKSRWQQG | |
| | NVFSCSVMHEALHNHYTQKSLSLSPGK (SEQ | |
| | ID NO: 119) | |
| h4A11. | EQQLQESGPGLVKPSETLSLTCTVTGFSLSSY | Same as v5 (SEQ ID NO: 110) |
| v7.9 | TVNWIRQPAGKGLEWIGYISYGGSAYYASW | |
| | ANGRFTISKDSSKNQVSLKLSSVTAADTAVY | |
| | FCARHMQVGGAPTGSMAAFDPWGPGTLVT | |
| | VSSASTKGPSVFPLAPSSKSTSGGTAALGCLV | |
| | KDYFPEPVTVSWNSGALTSGVHTFPAVLQSS | |
| | GLYSLSSVVTVPSSSLGTQTYICNVNHKPSNT | |
| | KVDKKVEPKSCDKTHTCPPCPAPELLGGPSV | |
| | FLFPPKPKDTLMISRTPEVTCVVVDVSHEDPE | |
| | VKFNWYVDGVEVHNAKTKPREEQYGSTYR | |
| | VVSVLTVLHQDWLNGKEYKCKVSNKALPA | |
| | PIEKTISKAKGQPREPQVYTLPPSREEMTKNQ | |
| | VSLTCLVKGFYPSDIAVEWESNGQPENNYKT | |
| | TPPVLDSDGSFFLYSKLTVDKSRWQQGNVFS | |
| | CSVMHEALHNHYTQKSLSLSPGK (SEQ ID | |
| | NO: 120) | |
| h4A11. | EQQLQESGPGLVKPSETLSLTCTVTGFSLSSY | Same as v5 (SEQ ID NO: 110) |
| v7.10 | TVNWVRQPAGKGLEWIGYISYGGSAYYAS | |
| | WANGRVTISKDSSKNQVSLKLSSVTAADTA | |
| | VYFCARHMQVGGAPTGSMAAFDPWGPGTL | |
| | VTVSSASTKGPSVFPLAPSSKSTSGGTAALGC | |
| | LVKDYFPEPVTVSWNSGALTSGVHTFPAVL | |
| | QSSGLYSLSSVVTVPSSSLGTQTYICNVNHKP | |
| | SNTKVDKKVEPKSCDKTHTCPPCPAPELLGG | |
| | PSVFLFPPKPKDTLMISRTPEVTCVVVDVSHE | |
| | DPEVKFNWYVDGVEVHNAKTKPREEQYGS | |
| | TYRVVSVLTVLHQDWLNGKEYKCKVSNKA | |
| | LPAPIEKTISKAKGQPREPQVYTLPPSREEMT | |
| | KNQVSLTCLVKGFYPSDIAVEWESNGQPEN | |
| | NYKTTPPVLDSDGSFFLYSKLTVDKSRWQQ | |
| | GNVFSCSVMHEALHNHYTQKSLSLSPGK | |
| | (SEQ ID NO: 121) | |
| h4A11. | EQQLQESGPGLVKPSETLSLTCTVTGFSLSSY | Same as v5 (SEQ ID NO: 110) |
| v7.11 | TVNWVRQPAGKGLEWIGYISYGGSAYYAS | |
| | WANGRFTISVDSSKNQVSLKLSSVTAADTAV | |
| | YFCARHMQVGGAPTGSMAAFDPWGPGTLV | |
| | TVSSASTKGPSVFPLAPSSKSTSGGTAALGCL | |
| | VKDYFPEPVTVSWNSGALTSGVHTFPAVLQS | |
| | SGLYSLSSVVTVPSSSLGTQTYICNVNHKPSN | |
| | TKVDKKVEPKSCDKTHTCPPCPAPELLGGPS | |
| | VFLFPPKPKDTLMISRTPEVTCVVVDVSHED | |
| | PEVKFNWYVDGVEVHNAKTKPREEQYGST | |
| | YRVVSVLTVLHQDWLNGKEYKCKVSNKAL | |
| | PAPIEKTISKAKGQPREPQVYTLPPSREEMTK | |
| | NQVSLTCLVKGFYPSDIAVEWESNGQPENN | |
| | YKTTPPVLDSDGSFFLYSKLTVDKSRWQQG | |
| | NVFSCSVMHEALHNHYTQKSLSLSPGK (SEQ | |
| | ID NO: 122) | |
| h4A11. | EQQLQESGPGLVKPSETLSLTCTVTGFSLSSY | Same as v5 (SEQ ID NO: 110) |
| v7.12 | TVNWVRQPAGKGLEWIGYISYGGSAYYAS | |
| | WANGRFTISKDTSKNQVSLKLSSVTAADTA | |
| | VYFCARHMQVGGAPTGSMAAFDPWGPGTL | |
| | VTVSSASTKGPSVFPLAPSSKSTSGGTAALGC | |
| | LVKDYFPEPVTVSWNSGALTSGVHTFPAVL | |
| | QSSGLYSLSSVVTVPSSSLGTQTYICNVNHKP | |
| | SNTKVDKKVEPKSCDKTHTCPPCPAPELLGG | |
| | PSVFLFPPKPKDTLMISRTPEVTCVVVDVSHE | |
| | DPEVKFNWYVDGVEVHNAKTKPREEQYGS | |
| | TYRVVSVLTVLHQDWLNGKEYKCKVSNKA | |
| | LPAPIEKTISKAKGQPREPQVYTLPPSREEMT | |
| | KNQVSLTCLVKGFYPSDIAVEWESNGQPEN | |
| | NYKTTPPVLDSDGSFFLYSKLTVDKSRWQQ | |
| | GNVFSCSVMHEALHNHYTQKSLSLSPGK | |
| | (SEQ ID NO: 123) | |
| h4A11. | EQQLQESGPGLVKPSETLSLTCTVTGFSLSSY | Same as v5 (SEQ ID NO: 110) |
| v7.13 | TVNWVRQPAGKGLEWIGYISYGGSAYYAS | |
| | WANGRFTISKDSSKNQFSLKLSSVTAADTAV | |
| | YFCARHMQVGGAPTGSMAAFDPWGPGTLV | |
| | TVSSASTKGPSVFPLAPSSKSTSGGTAALGCL | |
| | VKDYFPEPVTVSWNSGALTSGVHTFPAVLQS | |
| | SGLYSLSSVVTVPSSSLGTQTYICNVNHKPSN | |
| | TKVDKKVEPKSCDKTHTCPPCPAPELLGGPS | |
| | VFLFPPKPKDTLMISRTPEVTCVVVDVSHED | |
| | PEVKFNWYVDGVEVHNAKTKPREEQYGST | |
| | YRVVSVLTVLHQDWLNGKEYKCKVSNKAL | |
| | PAPIEKTISKAKGQPREPQVYTLPPSREEMTK | |
| | NQVSLTCLVKGFYPSDIAVEWESNGQPENN | |
| | YKTTPPVLDSDGSFFLYSKLTVDKSRWQQG | |
| | NVFSCSVMHEALHNHYTQKSLSLSPGK (SEQ | |
| | ID NO: 124) | |
| h4A11. | EQQLQESGPGLVKPSETLSLTCTVTGFSLSSY | Same as v5 (SEQ ID NO: 110) |
| v7.14 | TVNWVRQPAGKGLEWIGYISYGGSAYYAS | |
| | WANGRFTISKDSSKNQVSLKLSSVTAADTAV | |
| | YYCARHMQVGGAPTGSMAAFDPWGPGTLV | |
| | TVSSASTKGPSVFPLAPSSKSTSGGTAALGCL | |
| | VKDYFPEPVTVSWNSGALTSGVHTFPAVLQS | |
| | SGLYSLSSVVTVPSSSLGTQTYICNVNHKPSN | |
| | TKVDKKVEPKSCDKTHTCPPCPAPELLGGPS | |
| | VFLFPPKPKDTLMISRTPEVTCVVVDVSHED | |
| | PEVKFNWYVDGVEVHNAKTKPREEQYGST | |
| | YRVVSVLTVLHQDWLNGKEYKCKVSNKAL | |
| | PAPIEKTISKAKGQPREPQVYTLPPSREEMTK | |
| | NQVSLTCLVKGFYPSDIAVEWESNGQPENN | |
| | YKTTPPVLDSDGSFFLYSKLTVDKSRWQQG | |
| | NVFSCSVMHEALHNHYTQKSLSLSPGK (SEQ | |
| | ID NO: 125) | |
| h4A11. | EQQLQESGPGLVKPSETLSLTCTVTGFSLSSY | Same as v5 (SEQ ID NO: 110) |
| v7.15 | TVNWVRQPAGKGLEWIGYISYGGSAYYAS | |
| | WANGRFTISKDSSKNQVSLKLSSVTAADTAV | |
| | YFCARHMQVGGAPTGSMAAFDPWGQGTLV | |
| | TVSSASTKGPSVFPLAPSSKSTSGGTAALGCL | |
| | VKDYFPEPVTVSWNSGALTSGVHTFPAVLQS | |
| | SGLYSLSSVVTVPSSSLGTQTYICNVNHKPSN | |
| | TKVDKKVEPKSCDKTHTCPPCPAPELLGGPS | |
| | VFLFPPKPKDTLMISRTPEVTCVVVDVSHED | |
| | PEVKFNWYVDGVEVHNAKTKPREEQYGST | |
| | YRVVSVLTVLHQDWLNGKEYKCKVSNKAL | |
| | PAPIEKTISKAKGQPREPQVYTLPPSREEMTK | |
| | NQVSLTCLVKGFYPSDIAVEWESNGQPENN | |
| | YKTTPPVLDSDGSFFLYSKLTVDKSRWQQG | |
| | NVFSCSVMHEALHNHYTQKSLSLSPGK (SEQ | |
| | ID NO: 126) | |
| h4A11. | EVQLQESGPGLVKPSETLSLTCTVTGFSLSSY | EIVLTQSPGTLSLSPGERATL |
| v7.16 | TVNWIRQPAGKGLEWIGYISYGGSAYYASW | SCQSSQSVYNNNYLSWFQQ |
| | ANGRVTISKDTSKNQFSLKLSSVTAADTAVY | KPGQAPRLLIYGASTLTSGIP |
| | FCARHMQVGGAPTGSMAAFDPWGQGTLVT | DRFSGSGSGTDFTLTISRLEP |
| | VSSASTKGPSVFPLAPSSKSTSGGTAALGCLV | EDFAVYYCAGGYSGSSDKY |
| | KDYFPEPVTVSWNSGALTSGVHTFPAVLQSS | AFGGGTKVEIKRTVAAPSVFI |
| | GLYSLSSVVTVPSSSLGTQTYICNVNHKPSNT | FPPSDEQLKSGTASVVCLLN |
| | KVDKKVEPKSCDKTHTCPPCPAPELLGGPSV | NFYPREAKVQWKVDNALQS |
| | FLFPPKPKDTLMISRTPEVTCVVVDVSHEDPE | GNSQESVTEQDSKDSTYSLS |
| | VKFNWYVDGVEVHNAKTKPREEQYGSTYR | STLTLSKADYEKHKVYACE |
| | VVSVLTVLHQDWLNGKEYKCKVSNKALPA | VTHQGLSSPVTKSFNRGEC |
| | PIEKTISKAKGQPREPQVYTLPPSREEMTKNQ | (SEQ ID NO: 186) |
| | VSLTCLVKGFYPSDIAVEWESNGQPENNYKT | |
| | TPPVLDSDGSFFLYSKLTVDKSRWQQGNVFS | |
| | CSVMHEALHNHYTQKSLSLSPGK (SEQ ID | |
| | NO: 127) | |
| h4A11. | EVQLQESGPGLVKPSETLSLTCTVTGFSLSSY | Same as v7.16 (SEQ ID NO: |
| v7.17 | TVNWIRQPAGKGLEWIGYISYGGSAYYASW | 186) |
| | ANGRVTISKDTSKNQVSLKLSSVTAADTAVY | |
| | YCARHMQVGGAPTGSMAAFDPWGQGTLVT | |
| | VSSASTKGPSVFPLAPSSKSTSGGTAALGCLV | |
| | KDYFPEPVTVSWNSGALTSGVHTFPAVLQSS | |
| | GLYSLSSVVTVPSSSLGTQTYICNVNHKPSNT | |
| | KVDKKVEPKSCDKTHTCPPCPAPELLGGPSV | |
| | FLFPPKPKDTLMISRTPEVTCVVVDVSHEDPE | |
| | VKFNWYVDGVEVHNAKTKPREEQYGSTYR | |
| | VVSVLTVLHQDWLNGKEYKCKVSNKALPA | |
| | PIEKTISKAKGQPREPQVYTLPPSREEMTKNQ | |
| | VSLTCLVKGFYPSDIAVEWESNGQPENNYKT | |
| | TPPVLDSDGSFFLYSKLTVDKSRWQQGNVFS | |
| | CSVMHEALHNHYTQKSLSLSPGK (SEQ ID | |
| | NO: 128) | |
| h4A11. | EVQLQESGPGLVKPSETLSLTCTVTGFSLSSY | Same as v7.16 (SEQ ID NO: |
| v7.18 | TVNWIRQPAGKGLEWIGYISYGGSAYYASW | 186) |
| | ANGRVTISKDTSKNQVSLKLSSVTAADTAVY | |
| | FCARHMQVGGAPTGSMAAFDPWGQGTLVT | |
| | VSSASTKGPSVFPLAPSSKSTSGGTAALGCLV | |
| | KDYFPEPVTVSWNSGALTSGVHTFPAVLQSS | |
| | GLYSLSSVVTVPSSSLGTQTYICNVNHKPSNT | |
| | KVDKKVEPKSCDKTHTCPPCPAPELLGGPSV | |
| | FLFPPKPKDTLMISRTPEVTCVVVDVSHEDPE | |
| | VKFNWYVDGVEVHNAKTKPREEQYGSTYR | |
| | VVSVLTVLHQDWLNGKEYKCKVSNKALPA | |
| | PIEKTISKAKGQPREPQVYTLPPSREEMTKNQ | |
| | VSLTCLVKGFYPSDIAVEWESNGQPENNYKT | |
| | TPPVLDSDGSFFLYSKLTVDKSRWQQGNVFS | |
| | CSVMHEALHNHYTQKSLSLSPGK (SEQ ID | |
| | NO: 129) | |
| h4A11. | EVQLQESGPGLVKPSETLSLTCTVTGFSLSSY | Same as v7.16 (SEQ ID NO: |
| v7.19 | TVNWIRQPAGKGLEWIGYISYGGSAYYASW | 186) |
| | ANGRVTISKDTSQVSLKLSSVTAADTAVYFC | |
| | ARHMQVGGAPTGSMAAFDPWGQGTLVTVS | |
| | SASTKGPSVFPLAPSSKSTSGGTAALGCLVK | |
| | DYFPEPVTVSWNSGALTSGVHTFPAVLQSSG | |
| | LYSLSSVVTVPSSSLGTQTYICNVNHKPSNTK | |
| | VDKKVEPKSCDKTHTCPPCPAPELLGGPSVF | |
| | LFPPKPKDTLMISRTPEVTCVVVDVSHEDPE | |
| | VKFNWYVDGVEVHNAKTKPREEQYGSTYR | |
| | VVSVLTVLHQDWLNGKEYKCKVSNKALPA | |
| | PIEKTISKAKGQPREPQVYTLPPSREEMTKNQ | |
| | VSLTCLVKGFYPSDIAVEWESNGQPENNYKT | |
| | TPPVLDSDGSFFLYSKLTVDKSRWQQGNVFS | |
| | CSVMHEALHNHYTQKSLSLSPGK (SEQ ID | |
| | NO: 130) | |
| h4A11. | Same as v7.3 (SEQ ID NO: 114) | Same as v6 (SEQ ID NO: 111) |
| v8 | | |

### Example 9: Humanization of 6F12 (anti-TGFβ2)

### Materials and Methods

### Antibody Affinity Measurement by BIAcore

Binding affinity determinations of all humanized 4A11 antibodies were performed using SPR measurement with a BIAcore™-T200 instrument, as described above in Example 6 (2A11 Ab example).

### HEKBlue Cell-Based TGFβ Blocking Assay

HEKBlue cell-based TGFβ3 blocking assay was performed as described above in Example 6.

### Results:

6F12 humanization step 1: Rat 6F12 CDRs grafting into human frameworks Monoclonal antibody 6F12 was humanized as described below. Residue numbers are according to Kabat et al. supra.

The variable regions of the rat 6F12 (r6F12) antibody were first aligned with all human germlines. Two light chain human germlines (IGKV1-5, IGKV3-15) and two heavy chain human germlines (IGHV2-26, IGHV4-59) were identified as the closest acceptor frameworks for grafting r6F12 light chain and heavy chain hypervariable regions (CDRs) respectively. Specifically, from r6F12 light chain variable domain (VL), positions 24-34 (L1), 50-56 (L2) and 89-97 (L3) were grafted into IGKV1-5 and IGKV3-15. From r6F12 heavy chain variable domain (VH), positions 26-35 (H1), 50-65 (H2) and 95-102 (H3) were grafted into IGHV2-26 and IGHV4-59 respectively.

Furthermore, total four humanized variants were generated by shuffling different grafting acceptor frameworks: Humanized 6F12 version 1 (h6F12.v1) is by IGKV1-5 and IGHV2-26; humanized 6F12 version 2 (h6F12.v2) is by IGKV1-5 and IGHV4-59; humanized 6F12 version 3 (h6F12.v3) is by IGKV3-15 and IGHV2-26; humanized 6F12 version 4 (h6F12.v4) is by IGKV3-15 and IGHV4-59. In addition to CDRs graft onto the closest acceptor frameworks described above, for light chain framework, h6F12.v1 and h6F12.v2, used the same IGKV1-5 acceptor framework, retained rat residues at position 43 in framework II and position 66, 69, 71 and 87 in framework III of VL from r6F12; h6F12.v3 and h6F12.v4, used the same IGKV3-15 acceptor framework, retained rat residues at position 43 in framework II and position 58, 66, 69, 71 and 87 in framework III of VL from r6F12. For heavy chain framework, h6F12.v1 and h6F12.v3, used the same IGHV2-26 acceptor framework, retained rat residues at position 37, 48 and 49 in framework II and position 105 in framework IV of VH from r6F12; h6F12.v2 and h6F12.v4, used the same IGHV4-59 acceptor framework, retained rat residues at position 37 and 48 in framework II, position 67, 71 and 78 in framework III and position 105 in framework IV of VH from r6F12. All of these residues were found to be the part of framework residues acting as “Vernier” zone, which may adjust CDR structure and fine-tune the antigen fit. See, e.g., Foote and Winter, J. Mol. Biol. 224: 487-499 (1992). These CDR definitions include positions defined by their sequence hypervariability (Wu, T. T. & Kabat, E. A. (1970)), their structural location (Chothia, C. & Lesk, A. M. (1987)) and their involvement in antigen-antibody contacts (MacCallum et al. J. Mol. Biol. 262: 732-745 (1996)).

All 4 humanized 6F12 variants were further subject to BIAcore for human TGFβ isoforms binding affinity determination and HEKBlue cell-based assay for human TGFβ isoforms blocking potency assessment. In BIAcore analysis, although all 4 humanized 6F12 variants showed selective and similar binding to human TGFβ2 comparable to parental r6F12, the binding affinities were beyond the instrument detection limit, and therefore the results could not be used to determine which variant to move forward. Interestingly, in HEKBlue cell-based potency assay, the results indicated there were about 2.5-5 fold drop in blocking human TGFβ2 for all variants, except for h6F12.v3 (Table 21).

TABLE 21
6F12 humanization step 1 variants characterization summary

| 6F12 | | | | | | | |
|---|---|---|---|---|---|---|---|
| humanized | Human | BIAcore (pM) | | | HEKBlue TGFP blocking IC50 (nM) | | |
| variants | Framework | TGFβ1 | TGFβ2 | TGFβ3 | TGFβ1 | TGFβ2 | TGFβ3 |
| r6F12 | — | >5000 | <0.5 | >5000 | >200 | 0.004 | >200 |
| h6F12.v1 | K₁₋₅H₂₋₂₆ | >5000 | <0.5 | >5000 | >200 | 0.02 | >200 |
| h6F12.v2 | K₁₋₅H₄₋₅₉ | >5000 | <0.5 | >5000 | >200 | 0.01 | >200 |
| h6F12.v3 | K₃₋₁₅H₂₋₂₆ | >5000 | <0.5 | >5000 | >200 | 0.005 | >200 |
| h6F12.v4 | K₃₋₁₅H₄₋₅₉ | >5000 | <0.5 | >5000 | >200 | 0.02 | >200 |

### 6F12 Humanization Step 2: H6F12.v1 Framework Polishing

In humanized 6F12 framework polishing, more humanized 6F12 variants were generated by substituting each rat residue of h6F12.v1 at Vernier zone with corresponding human residue from germline sequence. Each variant was not evaluated by BIAcore due to instrument limitations, but by HEKBlue cell-based assay to determine human TGFβ isoform blocking potency.

These framework polishing variants are h6F12.v1.1 (VL: S43A), h6F12.v1.2 (VL: E66G), h6F12.v1.3 (VL: P69T), h6F12.v1.4 (VL: Y71F), h6F12.v1.5 (VL: F87Y), h6F12.v1.6 (VH: V37I), h6F12.v1.7 (VH: M48L), h6F12.v1.8 (VH: G49A) and h6F12.v1.9 (VH: P105R).

Most of the variants appeared to be similar or slightly improved in comparison with parental h6F12.v1 in the potency assay, except for the major impact on h6F12.v1.6 (VH: V37I) and h6F12.v1.9 (VH: P105R) with some drop in TGFβ2 blocking about 5-8 fold. There were two variants with minor impact: h6F12.v1.5 (VL: F87Y) with 2-fold drop and h6F12.v1.8 (VH: G49A) with 4-fold drop in TGFβ2 blocking respectively (Table 22). In summary, we determined that all rat residues at Vernier zone of h6F12.v1 can be replaced with human germline residues, except for major position 37 in framework II and position 105 in framework IV of VH, and minor position 87 in framework III of VL and position 49 in framework II of VH.

TABLE 22
6F12 humanization step 2 variants characterization summary

| h6F12.v1 | Human | Framework | HEKBIue TGFβ blocking IC50 (nM) | | |
|---|---|---|---|---|---|
| variants | Framework | mutation | TGFβ1 | TGFβ2 | TGFβ3 |
| Rat 6F12 | | | >200 | 0.004 | >200 |
| h6F12.v1 | K₁₋₅ H₂₋₂₆ | | >200 | 0.02 | >200 |
| h6F12.v1.1 | K₁₋₅ H₂₋₂₆ | LC:S43A | >200 | 0.02 | >200 |
| h6F12.v1.2 | K₁₋₅ H₂₋₂₆ | LC:E66G | >200 | 0.02 | >200 |
| h6F12.v1.3 | K₁₋₅ H₂₋₂₆ | LC:P69T | >200 | 0.01 | >200 |
| h6F12.v1.4 | K₁₋₅ H₂₋₂₆ | LC:Y71F | >200 | 0.02 | >200 |
| h6F12.v1.5 | K₁₋₅ H₂₋₂₆ | LC:F87Y | >200 | 0.04 | >200 |
| h6F12.v1.6 | K₁₋₅ H₂₋₂₆ | HC:V37I | >200 | 0.11 | >200 |
| h6F12.v1.7 | K₁₋₅ H₂₋₂₆ | HC:M48L | >200 | 0.03 | >200 |
| h6F12.v1.8 | K₁₋₅ H₂₋₂₆ | HC:G49A | >200 | 0.08 | >200 |
| h6F12.v1.9 | K₁₋₅ H₂₋₂₆ | HC:P105R | >200 | 0.16 | >200 |

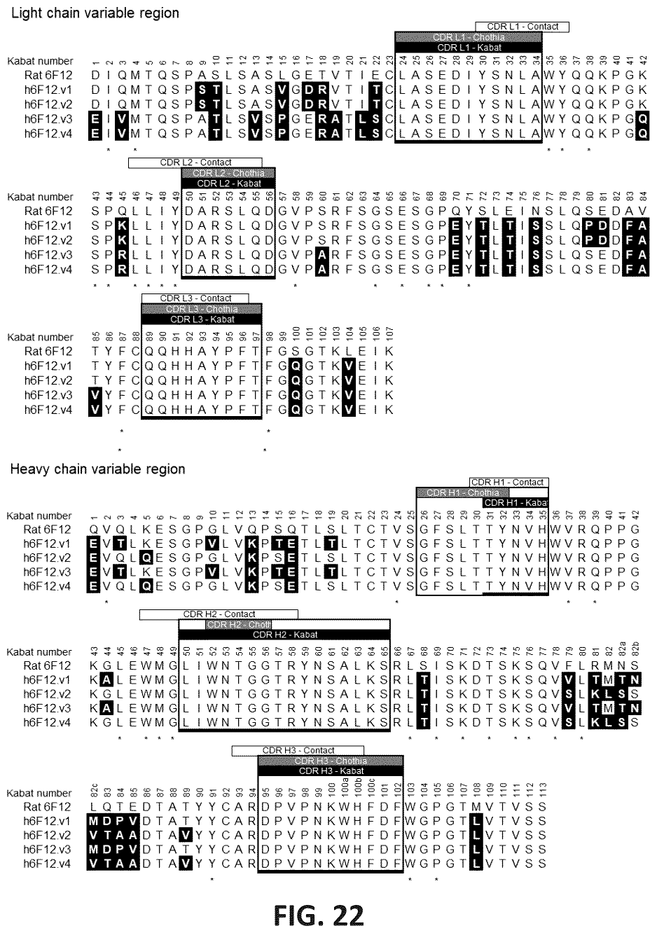

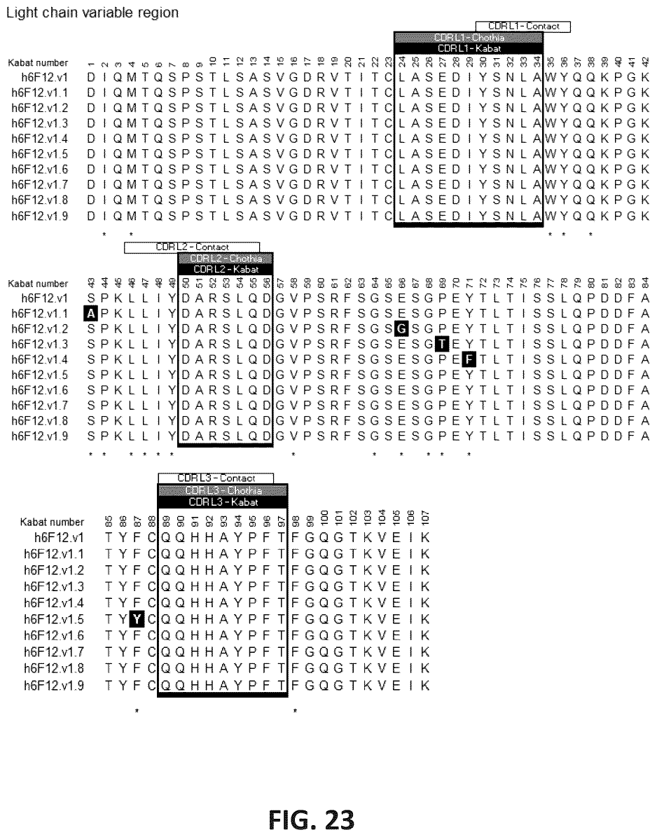

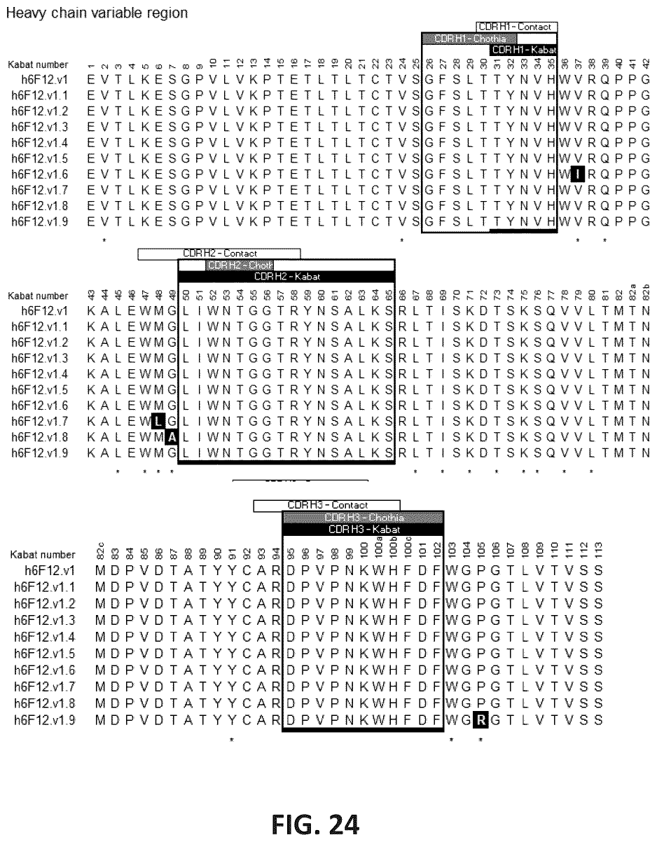

The sequences of the 6F12 variants are provided in Table 23 and Table 24, below, and sequence alignments are shown in FIG. 22, FIG. 23, and FIG. 24.

TABLE 23
Heavy and Light Chain Variable Region Amino Acid Sequences
of Humanized 6F12 Variants

| | VH | VL |
|---|---|---|
| h6F12. | EVTLKESGPVLVKPTETLTLTCTV | DIQMTQSPSTLSASVGDRVTITCL |
| v1 | SGFSLTTYNVHWVRQPPGKALE | ASEDIYSNLAWYQQKPGKSPKLLI |
| | WMGLIWNTGGTRYNSALKSRLTI | YDARSLQDGVPSRFSGSESGPEYT |
| | SKDTSKSQVVLTMTNMDPVDTA | LTISSLQPDDFATYFCQQHHAYPF |
| | TYYCARDPVPNKWHFDFWGPGT | TFGQGTKVEIK (SEQ ID NO: 131) |
| | LVTVSS (SEQ ID NO: 132) | |
| h6F12. | Same as v1 (SEQ ID NO: 132) | DIQMTQSPSTLSASVGDRVTITCL |
| v1.1 | | ASEDIYSNLAWYQQKPGKAPKLL |
| | | IYDARSLQDGVPSRFSGSESGPEY |
| | | TLTISSLQPDDFATYFCQQHHAYP |
| | | FTFGQGTKVEIK (SEQ ID NO: 133) |
| h6F12. | Same as v1 (SEQ ID NO: 132) | DIQMTQSPSTLSASVGDRVTITCL |
| v1.2 | | ASEDIYSNLAWYQQKPGKSPKLLI |
| | | YDARSLQDGVPSRFSGSGSGPEYT |
| | | LTISSLQPDDFATYFCQQHHAYPF |
| | | TFGQGTKVEIK (SEQ ID NO: 134) |
| h6F12. | Same as v1 (SEQ ID NO: 132) | DIQMTQSPSTLSASVGDRVTITCL |
| v1.3 | | ASEDIYSNLAWYQQKPGKSPKLLI |
| | | YDARSLQDGVPSRFSGSESGTEYT |
| | | LTISSLQPDDFATYFCQQHHAYPF |
| | | TFGQGTKVEIK (SEQ ID NO: 135) |
| h6F12. | Same as v1 (SEQ ID NO: 132) | DIQMTQSPSTLSASVGDRVTITCL |
| v1.4 | | ASEDIYSNLAWYQQKPGKSPKLLI |
| | | YDARSLQDGVPSRFSGSESGPEFT |
| | | LTISSLQPDDFATYFCQQHHAYPF |
| | | TFGQGTKVEIK (SEQ ID NO: 136) |
| h6F12. | Same as v1 (SEQ ID NO: 132) | DIQMTQSPSTLSASVGDRVTITCL |
| v1.5 | | ASEDIYSNLAWYQQKPGKSPKLLI |
| | | YDARSLQDGVPSRFSGSESGPEYT |
| | | LTISSLQPDDFATYYCQQHHAYPF |
| | | TFGQGTKVEIK (SEQ ID NO: 137) |
| h6F12. | EVTLKESGPVLVKPTETLTLTCTV | Same as v1 (SEQ ID NO: 131) |
| v1.6 | SGFSLTTYNVHWIRQPPGKALEW | |
| | MGLIWNTGGTRYNSALKSRLTIS | |
| | KDTSKSQVVLTMTNMDPVDTAT | |
| | YYCARDPVPNKWHFDFWGPGTL | |
| | VTVSS (SEQ ID NO: 138) | |
| h6F12. | EVTLKESGPVLVKPTETLTLTCTV | Same as v1 (SEQ ID NO: 131) |
| v1.7 | SGFSLTTYNVHWVRQPPGKALE | |
| | WLGLIWNTGGTRYNSALKSRLTI | |
| | SKDTSKSQVVLTMTNMDPVDTA | |
| | TYYCARDPVPNKWHFDFWGPGT | |
| | LVTVSS (SEQ ID NO: 139) | |
| h6F12. | EVTLKESGPVLVKPTETLTLTCTV | Same as v1 (SEQ ID NO: 131) |
| v1.8 | SGFSLTTYNVHWVRQPPGKALE | |
| | WMALIWNTGGTRYNSALKSRLTI | |
| | SKDTSKSQVVLTMTNMDPVDTA | |
| | TYYCARDPVPNKWHFDFWGPGT | |
| | LVTVSS (SEQ ID NO: 140) | |
| h6F12. | EVTLKESGPVLVKPTETLTLTCTV | Same as v1 (SEQ ID NO: 131) |
| v1.9 | SGFSLTTYNVHWVRQPPGKALE | |
| | WMGLIWNTGGTRYNSALKSRLTI | |
| | SKDTSKSQVVLTMTNMDPVDTA | |
| | TYYCARDPVPNKWHFDFWGRGT | |
| | LVTVSS (SEQ ID NO: 141) | |
| h6F12. | EVQLQESGPGLVKPSETLSLTCTV | Same as v1 (SEQ ID NO: 131) |
| v2 | SGFSLTTYNVHWVRQPPGKGLE | |
| | WMGLIWNTGGTRYNSALKSRLTI | |
| | SKDTSKSQVSLKLSSVTAADTAV | |
| | YYCARDPVPNKWHFDFWGPGTL | |
| | VTVSS (SEQ ID NO: 142) | |
| h6F12. | Same as v1 (SEQ ID NO: 132) | EIVMTQSPATLSVSPGERATLSCL |
| v3 | | ASEDIYSNLAWYQQKPGQSPRLLI |
| | | YDARSLQDGVPARFSGSESGPEY |
| | | TLTISSLQSEDFAVYFCQQHHAYP |
| | | FTFGQGTKVEIK (SEQ ID NO: 143) |
| h6F12. | Same as v2 (SEQ ID NO: 142) | EIVMTQSPATLSVSPGERATLSCL |
| v4 | | ASEDIYSNLAWYQQKPGQSPRLLI |
| | | YDARSLQDGVPARFSGSESGPEY |
| | | TLTISSLQSEDFAVYFCQQHHAYP |
| | | FTFGQGTKVEIK (SEQ ID NO: 144) |

TABLE 24
Complete Heavy and Light Chain Sequences for Humanized 6F12 Variants

| | Complete H Chain | Complete L Chain |
|---|---|---|
| h6F12. | EVTLKESGPVLVKPTETLTLTC | DIQMTQSPSTLSASVGDRVTITCLA |
| v1 | TVSGFSLTTYNVHWVRQPPGK | SEDIYSNLAWYQQKPGKSPKLLIYD |
| | ALEWMGLIWNTGGTRYNSAL | ARSLQDGVPSRFSGSESGPEYTLTIS |
| | KSRLTISKDTSKSQVVLTMTNM | SLQPDDFATYFCQQHHAYPFTFGQ |
| | DPVDTATYYCARDPVPNKWHF | GTKVEIKRTVAAPSVFIFPPSDEQLK |
| | DFWGPGTLVTVSSASTKGPSVF | SGTASVVCLLNNFYPREAKVQWKV |
| | PLAPSSKSTSGGTAALGCLVKD | DNALQSGNSQESVTEQDSKDSTYS |
| | YFPEPVTVSWNSGALTSGVHTF | LSSTLTLSKADYEKHKVYACEVTH |
| | PAVLQSSGLYSLSSVVTVPSSSL | QGLSSPVTKSFNRGEC (SEQ ID NO: |
| | GTQTYICNVNHKPSNTKVDKK | 145) |
| | VEPKSCDKTHTCPPCPAPELLG | |
| | GPSVFLFPPKPKDTLMISRTPEV | |
| | TCVVVDVSHEDPEVKFNWYV | |
| | DGVEVHNAKTKPREEQYGSTY | |
| | RVVSVLTVLHQDWLNGKEYK | |
| | CKVSNKALPAPIEKTISKAKGQ | |
| | PREPQVYTLPPSREEMTKNQVS | |
| | LTCLVKGFYPSDIAVEWESNGQ | |
| | PENNYKTTPPVLDSDGSFFLYS | |
| | KLTVDKSRWQQGNVFSCSVM | |
| | HEALHNHYTQKSLSLSPGK | |
| | (SEQ ID NO: 146) | |
| h6F12. | Same as v1 (SEQ ID NO: 146) | DIQMTQSPSTLSASVGDRVTITCLA |
| v1.1 | | SEDIYSNLAWYQQKPGKAPKLLIY |
| | | DARSLQDGVPSRFSGSESGPEYTLTI |
| | | SSLQPDDFATYFCQQHHAYPFTFGQ |
| | | GTKVEIKRTVAAPSVFIFPPSDEQLK |
| | | SGTASVVCLLNNFYPREAKVQWKV |
| | | DNALQSGNSQESVTEQDSKDSTYS |
| | | LSSTLTLSKADYEKHKVYACEVTH |
| | | QGLSSPVTKSFNRGEC (SEQ ID NO: |
| | | 147) |
| h6F12. | Same as v1 (SEQ ID NO: 146) | DIQMTQSPSTLSASVGDRVTITCLA |
| v1.2 | | SEDIYSNLAWYQQKPGKSPKLLIYD |
| | | ARSLQDGVPSRFSGSGSGPEYTLTIS |
| | | SLQPDDFATYFCQQHHAYPFTFGQ |
| | | GTKVEIKRTVAAPSVFIFPPSDEQLK |
| | | SGTASVVCLLNNFYPREAKVQWKV |
| | | DNALQSGNSQESVTEQDSKDSTYS |
| | | LSSTLTLSKADYEKHKVYACEVTH |
| | | QGLSSPVTKSFNRGEC (SEQ ID NO: |
| | | 148) |
| h6F12. | Same as v1 (SEQ ID NO: 146) | DIQMTQSPSTLSASVGDRVTITCLA |
| v1.3 | | SEDIYSNLAWYQQKPGKSPKLLIYD |
| | | ARSLQDGVPSRFSGSESGTEYTLTIS |
| | | SLQPDDFATYFCQQHHAYPFTFGQ |
| | | GTKVEIKRTVAAPSVFIFPPSDEQLK |
| | | SGTASVVCLLNNFYPREAKVQWKV |
| | | DNALQSGNSQESVTEQDSKDSTYS |
| | | LSSTLTLSKADYEKHKVYACEVTH |
| | | QGLSSPVTKSFNRGEC (SEQ ID NO: |
| | | 149) |
| h6F12. | Same as v1 (SEQ ID NO: 146) | DIQMTQSPSTLSASVGDRVTITCLA |
| v1.4 | | SEDIYSNLAWYQQKPGKSPKLLIYD |
| | | ARSLQDGVPSRFSGSESGPEFTLTIS |
| | | SLQPDDFATYFCQQHHAYPFTFGQ |
| | | GTKVEIKRTVAAPSVFIFPPSDEQLK |
| | | SGTASVVCLLNNFYPREAKVQWKV |
| | | DNALQSGNSQESVTEQDSKDSTYS |
| | | LSSTLTLSKADYEKHKVYACEVTH |
| | | QGLSSPVTKSFNRGEC (SEQ ID NO: |
| | | 150) |
| h6F12. | Same as v1 (SEQ ID NO: 146) | DIQMTQSPSTLSASVGDRVTITCLA |
| v1.5 | | SEDIYSNLAWYQQKPGKSPKLLIYD |
| | | ARSLQDGVPSRFSGSESGPEYTLTIS |
| | | SLQPDDFATYYCQQHHAYPFTFGQ |
| | | GTKVEIKRTVAAPSVFIFPPSDEQLK |
| | | SGTASVVCLLNNFYPREAKVQWKV |
| | | DNALQSGNSQESVTEQDSKDSTYS |
| | | LSSTLTLSKADYEKHKVYACEVTH |
| | | QGLSSPVTKSFNRGEC (SEQ ID NO: |
| | | 151) |
| h6F12. | EVTLKESGPVLVKPTETLTLTC | Same as v1 (SEQ ID NO: 145) |
| v1.6 | TVSGFSLTTYNVHWIRQPPGKA | |
| | LEWMGLIWNTGGTRYNSALKS | |
| | RLTISKDTSKSQVVLTMTNMDP | |
| | VDTATYYCARDPVPNKWHFDF | |
| | WGPGTLVTVSSASTKGPSVFPL | |
| | APSSKSTSGGTAALGCLVKDYF | |
| | PEPVTVSWNSGALTSGVHTFPA | |
| | VLQSSGLYSLSSVVTVPSSSLGT | |
| | QTYICNVNHKPSNTKVDKKVE | |
| | PKSCDKTHTCPPCPAPELLGGP | |
| | SVFLFPPKPKDTLMISRTPEVTC | |
| | VVVDVSHEDPEVKFNWYVDG | |
| | VEVHNAKTKPREEQYGSTYRV | |
| | VSVLTVLHQDWLNGKEYKCK | |
| | VSNKALPAPIEKTISKAKGQPR | |
| | EPQVYTLPPSREEMTKNQVSLT | |
| | CLVKGFYPSDIAVEWESNGQPE | |
| | NNYKTTPPVLDSDGSFFLYSKL | |
| | TVDKSRWQQGNVFSCSVMHE | |
| | ALHNHYTQKSLSLSPGK (SEQ | |
| | ID NO: 152) | |
| h6F12. | EVTLKESGPVLVKPTETLTLTC | Same as v1 (SEQ ID NO: 145) |
| v1.7 | TVSGFSLTTYNVHWVRQPPGK | |
| | ALEWLGLIWNTGGTRYNSALK | |
| | SRLTISKDTSKSQVVLTMTNMD | |
| | PVDTATYYCARDPVPNKWHFD | |
| | FWGPGTLVTVSSASTKGPSVFP | |
| | LAPSSKSTSGGTAALGCLVKDY | |
| | FPEPVTVSWNSGALTSGVHTFP | |
| | AVLQSSGLYSLSSVVTVPSSSL | |
| | GTQTYICNVNHKPSNTKVDKK | |
| | VEPKSCDKTHTCPPCPAPELLG | |
| | GPSVFLFPPKPKDTLMISRTPEV | |
| | TCVVVDVSHEDPEVKFNWYV | |
| | DGVEVHNAKTKPREEQYGSTY | |
| | RVVSVLTVLHQDWLNGKEYK | |
| | CKVSNKALPAPIEKTISKAKGQ | |
| | PREPQVYTLPPSREEMTKNQVS | |
| | LTCLVKGFYPSDIAVEWESNGQ | |
| | PENNYKTTPPVLDSDGSFFLYS | |
| | KLTVDKSRWQQGNVFSCSVM | |
| | HEALHNHYTQKSLSLSPGK | |
| | (SEQ ID NO: 153) | |
| h6F12. | EVTLKESGPVLVKPTETLTLTC | Same as v1 (SEQ ID NO: 145) |
| v1.8 | TVSGFSLTTYNVHWVRQPPGK | |
| | ALEWMALIWNTGGTRYNSAL | |
| | KSRLTISKDTSKSQVVLTMTNM | |
| | DPVDTATYYCARDPVPNKWHF | |
| | DFWGPGTLVTVSSASTKGPSVF | |
| | PLAPSSKSTSGGTAALGCLVKD | |
| | YFPEPVTVSWNSGALTSGVHTF | |
| | PAVLQSSGLYSLSSVVTVPSSSL | |
| | GTQTYICNVNHKPSNTKVDKK | |
| | VEPKSCDKTHTCPPCPAPELLG | |
| | GPSVFLFPPKPKDTLMISRTPEV | |
| | TCVVVDVSHEDPEVKFNWYV | |
| | DGVEVHNAKTKPREEQYGSTY | |
| | RVVSVLTVLHQDWLNGKEYK | |
| | CKVSNKALPAPIEKTISKAKGQ | |
| | PREPQVYTLPPSREEMTKNQVS | |
| | LTCLVKGFYPSDIAVEWESNGQ | |
| | PENNYKTTPPVLDSDGSFFLYS | |
| | KLTVDKSRWQQGNVFSCSVM | |
| | HEALHNHYTQKSLSLSPGK | |
| | (SEQ ID NO: 154) | |
| h6F12. | DIQMTQSPSTLSASVGDRVTIT | Same as v1 (SEQ ID NO: 145) |
| v1.9 | CLASEDIYSNLAWYQQKPGKS | |
| | PKLLIYDARSLQDGVPSRFSGS | |
| | ESGPEYTLTISSLQPDDFATYFC | |
| | QQHHAYPFTFGQGTKVEIKRT | |
| | VAAPSVFIFPPSDEQLKSGTASV | |
| | VCLLNNFYPREAKVQWKVDN | |
| | ALQSGNSQESVTEQDSKDSTYS | |
| | LSSTLTLSKADYEKHKVYACE | |
| | VTHQGLSSPVTKSFNRGEC | |
| | (SEQ ID NO: 155) | |
| h6F12. | EVQLQESGPGLVKPSETLSLTC | Same as v1 (SEQ ID NO: 145) |
| v2 | TVSGFSLTTYNVHWVRQPPGK | |
| | GLEWMGLIWNTGGTRYNSAL | |
| | KSRLTISKDTSKSQVSLKLSSVT | |
| | AADTAVYYCARDPVPNKWHF | |
| | DFWGPGTLVTVSSASTKGPSVF | |
| | PLAPSSKSTSGGTAALGCLVKD | |
| | YFPEPVTVSWNSGALTSGVHTF | |
| | PAVLQSSGLYSLSSVVTVPSSSL | |
| | GTQTYICNVNHKPSNTKVDKK | |
| | VEPKSCDKTHTCPPCPAPELLG | |
| | GPSVFLFPPKPKDTLMISRTPEV | |
| | TCVVVDVSHEDPEVKFNWYV | |
| | DGVEVHNAKTKPREEQYGSTY | |
| | RVVSVLTVLHQDWLNGKEYK | |
| | CKVSNKALPAPIEKTISKAKGQ | |
| | PREPQVYTLPPSREEMTKNQVS | |
| | LTCLVKGFYPSDIAVEWESNGQ | |
| | PENNYKTTPPVLDSDGSFFLYS | |
| | KLTVDKSRWQQGNVFSCSVM | |
| | HEALHNHYTQKSLSLSPGK | |
| | (SEQ ID NO: 156) | |
| h6F12. | Same as v1 (SEQ ID NO: 146) | EIVMTQSPATLSVSPGERATLSCLA |
| v3 | | SEDIYSNLAWYQQKPGQSPRLLIYD |
| | | ARSLQDGVPARFSGSESGPEYTLTIS |
| | | SLQSEDFAVYFCQQHHAYPFTFGQ |
| | | GTKVEIKRTVAAPSVFIFPPSDEQLK |
| | | SGTASVVCLLNNFYPREAKVQWKV |
| | | DNALQSGNSQESVTEQDSKDSTYS |
| | | LSSTLTLSKADYEKHKVYACEVTH |
| | | QGLSSPVTKSFNRGEC (SEQ ID NO: |
| | | 157) |
| h6F12. | Same as v2 (SEQ ID NO: 156) | EIVMTQSPATLSVSPGERATLSCLA |
| v4 | | SEDIYSNLAWYQQKPGQSPRLLIYD |
| | | ARSLQDGVPARFSGSESGPEYTLTIS |
| | | SLQSEDFAVYFCQQHHAYPFTFGQ |
| | | GTKVEIKRTVAAPSVFIFPPSDEQLK |
| | | SGTASVVCLLNNFYPREAKVQWKV |
| | | DNALQSGNSQESVTEQDSKDSTYS |
| | | LSSTLTLSKADYEKHKVYACEVTH |
| | | QGLSSPVTKSFNRGEC (SEQ ID NO: |
| | | 158) |

### Example 10: Structural Characterization of Isoform-Selective Anti-TGFβ Antibodies

### Materials and Methods

Fabs were expressed from CHO cells. Human TGFβ2 and TGFβ3 were purchased from Peprotech (Rocky Hill, NJ). Fabs and TGFβ2/3 were mixed at 1:1.5 ratio on ice for 30 minutes, complexes were isolated from HiLoad Superdex S200 16/600 column (GE healthcare life science) in 25 mM Tris pH7.5, and 50 mM NaCl. Samples were concentrated to 10 mg/ml for crystallization. Vapor diffusion method was used for growing crystals. 1 μl of protein was mixed with 1 μl mother liquid over 500 μl well solution. 2A10 TGFβ3 was crystallized in 25% PEG 1000, and 0.1M Hepes pH 7.5. The 4A11v2/TGFβ2 complex was crystallized in 15% PEG 4000, and 0.1 M sodium cacodylate pH 6. 4A11v7 and TGFβ2 complex was crystallized in 30% PEG 1000, and 0.1M Tris pH8.5. Crystals were cryo-protected with 20% glycerol and frozen in liquid nitrogen before data collection.

### Results:

### 2A10/β3

The crystal structure of the 2A10v4 Fab (expressed in CHO) and mature dimeric form of human TGFβ3 (A301-S412, expressed in E. coli, purchased from Peprotech (Rocky Hill, NJ)) was solved at 2.5 Å with R/Rfree refinement statistics of 22.6% and 27.1% respectively (Table 25).

TABLE 25
Data collection and refinement statistics
for TGFβ3/2A10v4 structure

| | TGFβ3/2A10v4 |
|---|---|
| Data collection | APS 24-IDC |
| Space group | I2 |
| Cell dimensions | |
| a, b, c (Å) | 133.94, 47.20, 200.83 |
| α, β γ (°) | 90, 101.68, 90 |
| Resolution (Å) | 49.17-2.50 (2.59-2.50) |
| R_{sym} or R_{merge} | 0.109 (1.367) |
| I/σI | 11.0 (1.1) |
| Completeness (%) | 99.3 (98.9) |
| Redundancy | 5.6 (5.4) |
| CC½ | 0.996 (0.514) |
| Refinement | |
| Resolution (Å) | 49.17-2.50 |
| No. reflections (total/test) | 42938/2096 |
| R_{work}/R_{free} | 22.3/26.9% |
| No. atoms | |
| Protein | 7869 |
| Water | 161 |
| B-factors | |
| Protein | 62.6 |
| Water | 47.8 |
| R.m.s. deviations | |
| Bond lengths (Å) | 0.010 |
| Bond angles (°) | 1.22 |

*Values in parentheses are for highest-resolution shell.

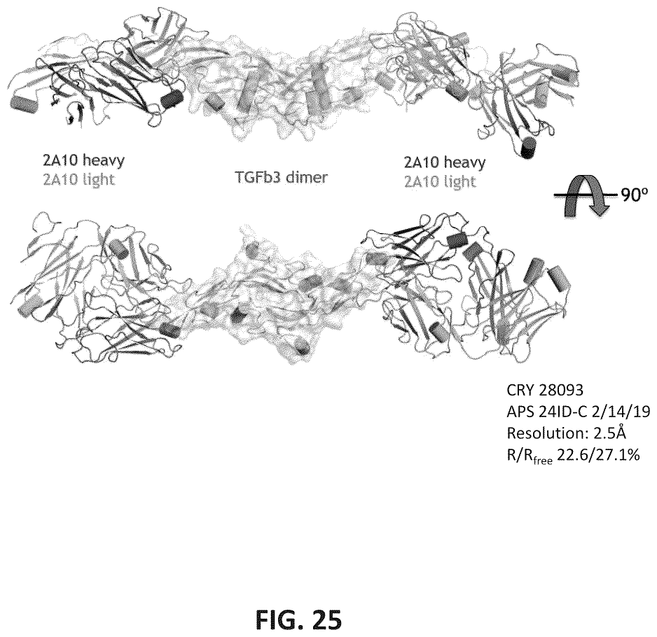

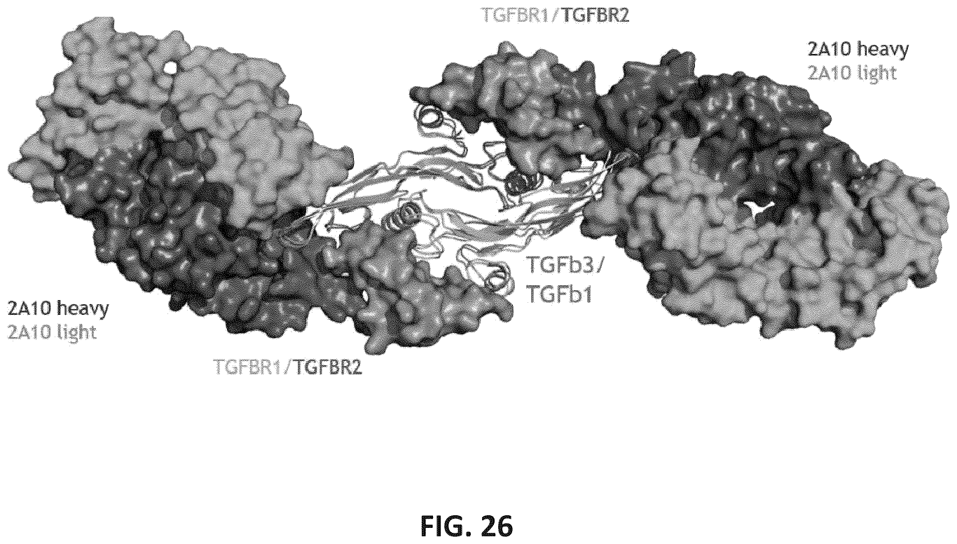

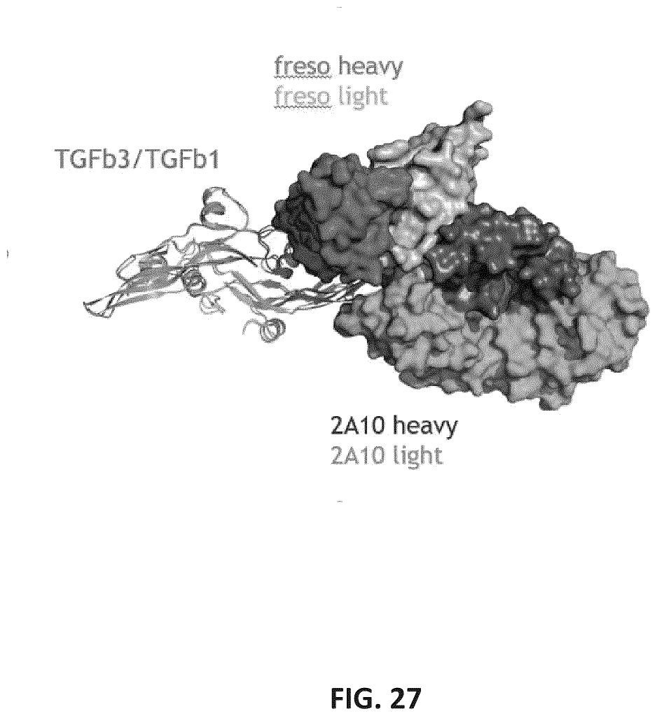

The structure in FIG. 25 shows that each dimer of TGFβ33 is bound by two 2A10 Fabs. 2A10 binds to the beta6/beta7 hairpin region of TGFβ33. Comparison of the 2A10/TGFβ33 structure with the structure of TGFβ1 bound to TGFBR1 and TGFBR2 (FIG. 26) (see also, Radaev JBC 2011, PDB 3KFD) indicates that the 2A10 Fab would sterically block the ability of TGFBR2, but not TGFBR1, to bind TGFβ33. Since the signaling complex of TGFβ3 with TGFBR1 and TGFBR2 is known to cooperatively assemble, first by TGFβ3 binding to TGFBR2 followed by recruitment of TGFBR1, it is expected that blocking TGFBR2 binding is sufficient to completely prevent the R1/R2 signaling receptors from binding to TGFβ33. The binding mode of 2A10 is different from the pan-TGFβ antibody fresolimumab, which binds to all TGFβ isoforms via an epitope that blocks both TGFBR1 and TGFBR2 binding (PDB ID 4KV5, 4KXZ, 3E01, 3KFD).

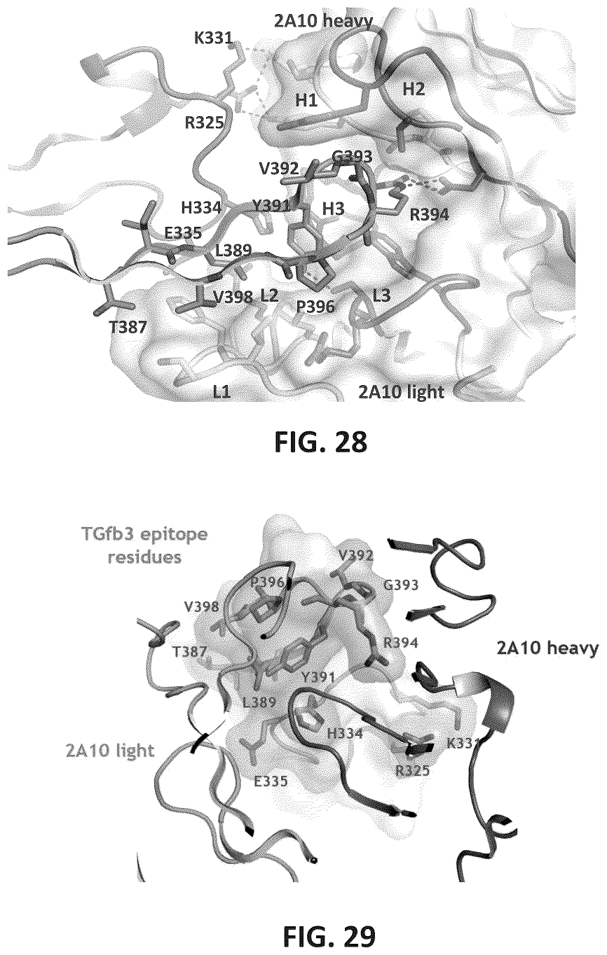

The epitope on TGFβ3 bound by 2A10 is shown in FIG. 28 and FIG. 29, and summarized in a list of contacts found in Table 26. TGFB3 residues contacted by 2A10 (within 5.0 Å) are R325, K331, W332, H334, E335, T387, I388, L389, Y391, V392, G393, R394, P396, K397, and V398. As one can see from FIG. 28, the bulk of the epitope is contributed by the beta6 and beta7 strands of TGFβ3, including the beta hairpin region. This beta hairpin contains R394, which makes the largest number of contacts with 2A10 (see Table 26) and also makes an ionic salt bridge with Asp50 in the 2A10 heavy chain CDR2. The TGFβ3 epitope is shown from another angle in FIG. 29, which better highlights the contacts between 2A10 and the 325-335 loop in TGFβ3.

TABLE 26
Contact Residues on TGFβ3 and
anti-TGFβ3 mAb 2A10

| TGFβ3 | | |
|---|---|---|
| Amino Acid | Light | Heavy |
| Residue | Chain | Chain |
| R325 | | Y32 |
| K331 | | D28 |
| | | S31 |
| W332 | | Y53 |
| H334 | R50 | P96 |
| | | G97 |
| | | G98 |
| E335 | F30 | |
| | R50 | |
| | N53 | |
| T387 | S28 | |
| | R29 | |
| | F30 | |
| I388 | F30 | |
| L389 | 127d | |
| | F30 | |
| | L32 | |
| Y391 | L32 | Y53 |
| | H34 | P96 |
| | S91 | G97 |
| | W96 | G98 |
| V392 | | Y53 |
| G393 | | V52 |
| | | Y53 |
| R394 | S91 | G33 |
| | S94 | M34 |
| | W96 | S35 |
| | | D50 |
| | | I51 |
| | | V52 |
| | | Y58 |
| | | A95 |
| | | G97 |
| P396 | I27d | |
| | L32 | |
| | S91 | |
| | R92 | |
| | E93 | |
| K397 | I27d | |
| V398 | I27d | |
| | S28 | |
| | F30 | |

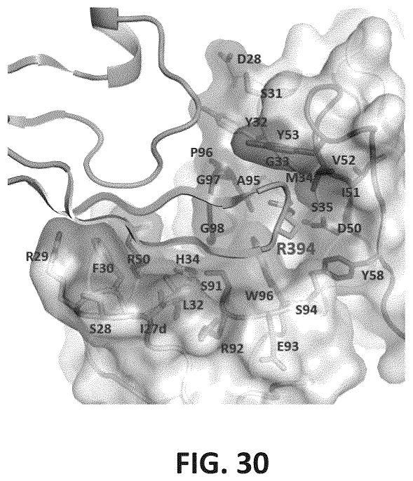

The 2A10 paratope is shown in FIG. 30. Both the heavy and light chains contribute relatively equal surface area to binding of TGFβ3. ~885 Å2 of buried surface area (BSA) is contributed by the light chain, and ~805 Å2 of BSA is contributed by the heavy chain, for a total of ~1690 Å2 in the interface between 2A10 and TGFβ3 (an average of the two Fabs bound to the dimer in the structure). Given the concave surface of the antibody paratope combined with extended beta6/beta7 epitope on TGFβ3, all six CDRs contact TGFβ3 (FIG. 30 and Table 26). TGFβ3 Arg394, which makes a key contact with Asp50 in the 2A10 heavy chain, is almost completely buried in the center of the antibody paratope, contacting 2A10 light chain CDR H3, and all three CDRs from the heavy chain. An extended 15 residue light chain CDR L1 is a key contributor to interactions with the beta6/beta7 strands, and short glycine rich CDR H3 helps create the space for the beta6/beta7 hairpin to approach and interact with CDR H2 D50. These unique interactions help explain the fact that the light chain BSA is larger than the heavy chain BSA, which is somewhat unusual for antibody antigen interactions.

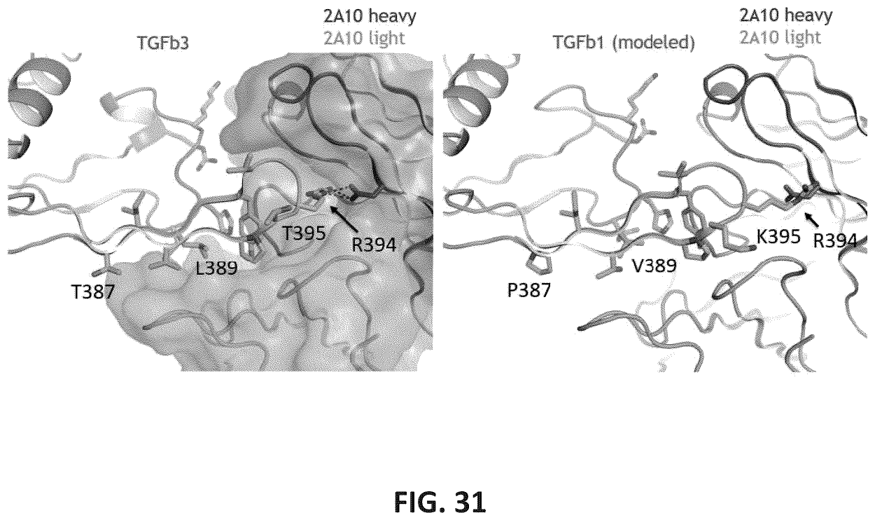

Selectivity for TGFβ3 over TGFβ1 and TGFβ2 is likely achieved in different ways for each of these isoforms. For human TGFβ1, there are three amino acid differences between TGFβ1 and TGFβ3 near or within the 2A10 epitope. These residues, T387P (meaning the residue is a “T” in TGFβ3 (human TGFβ3 numbering) and a P in TGFβ1 (P365 in TGFβ1)), L389V, and T395K suggest that residues along the beta6 strands (T387P, L389V) play a large role in 2A10 binding (FIG. 31). In TGFβ3, T395 does not make a direct interaction with 2A10 in the crystal structure, so the effect of the change to lysine in this residue is unclear.

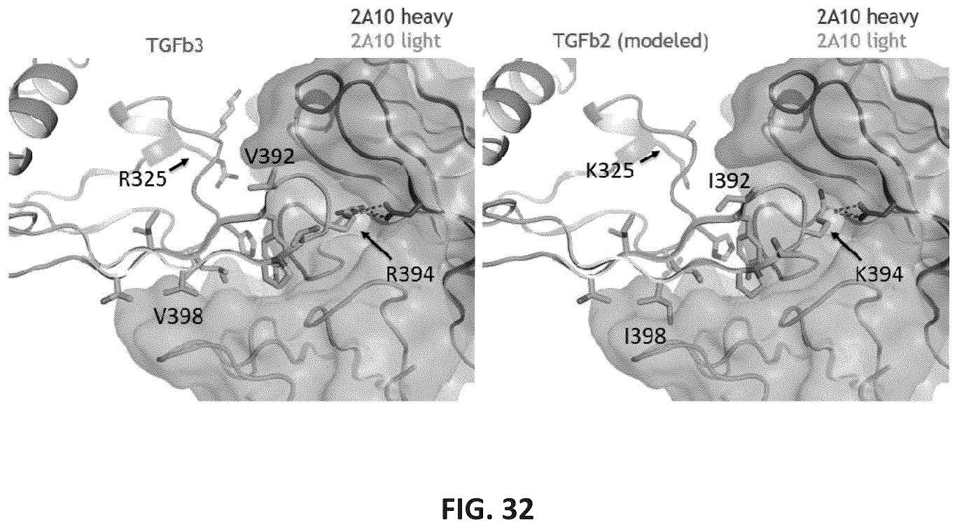

For TGFβ2, there are four amino acid changes between TGFβ2 and TGFβ3 that lie within or near the 2A10 epitope. Of these four residues (R325K (meaning the residue is a “R” in TGFβ3 and a K in TGFβ2 (human TGFβ3 numbering), V392I, R394K, V398I), three could disrupt productive contact with 2A10 (FIG. 32). TGFβ3 R325 makes a hydrogen bond with heavy chain residue Y32, which would be altered when changed to a lysine. A change to isoleucine at position V398 could introduce a steric clash with 2A10. Most importantly, a change in the highly contacted R394 residue (to lysine in TGFβ2) would also likely change the chemistry of the interaction with 2A10 in an unfavorable manner. The substitution of isoleucine for valine at position 392 would not be expected to have a large effect on binding, given its position in the epitope and the conservative nature of the change. Overall, the structure of 2A10 bound to TGFβ3 provides a strong indication of how 2A10 achieves high selectivity between TGFβ isoforms.

### 4A11/TGFβ2

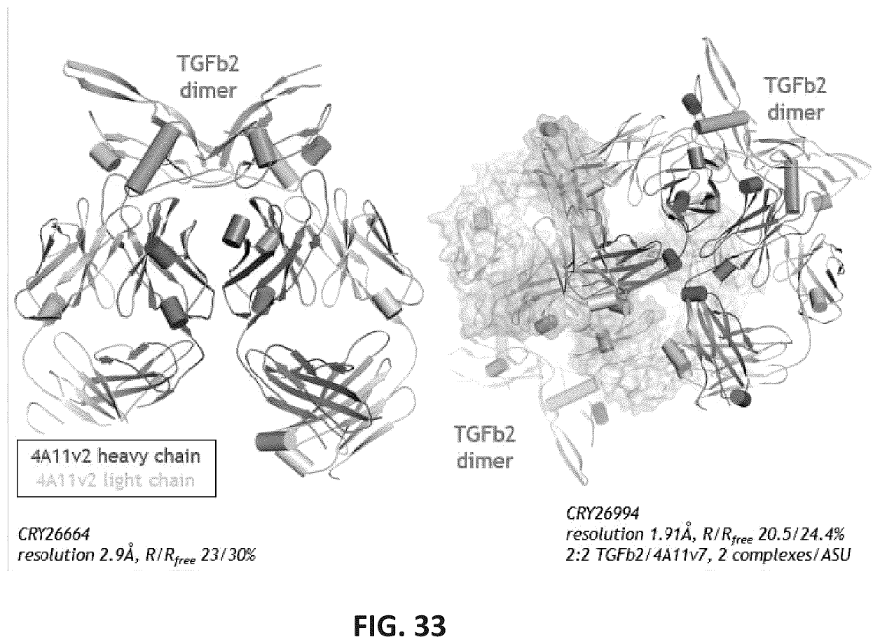

In order to better understand the reasons for functional differences between the humanized 4A11 clones, the structures of two versions of 4A11, v2 and v7, bound to the mature form of human TGFβ2 were solved using methods as described above with respect to 2A10. V2 and v7 were humanized to the VL1VH3 and VL3VH4 frameworks respectively. FIG. 33 shows the two structures. 4A11v2/TGFβ2 was solved at 2.9 Å resolution, to R/Rfree statistics of 23/30%, while 4A11v7/TGFβ2 was solved at 1.91 Å with R/Rfree statistics of 20.5/24.4% (Table 27). Each TGFβ2 dimer is bound by two 4A11 Fabs that share a nearly identical epitope. While the overall architecture of the complexes was very similar, 4A11v7 crystallized in a different spacegroup and there were some small differences in elbow angle for the second Fab bound to TGFβ2.

TABLE 27
Data collection and refinement statistics for TGFβ2/4A11 structures

| | TGFβ2/4A11v2 | TGFβ2/4A11v7 |
|---|---|---|
| Data collection | ALS 5.0.2 | APS 22ID |
| Space group | P2 21 21 | P1 |
| Cell dimensions | | |
| a, b, c (Å) | 60.15, 103.48, | 64.42, 85.11, 112.73 |
| | 196.25 | |
| a, β, γ (°) | 90, 90, 90 | 112.73, 99.15, 97.32 |
| Resolution (Å) | 49.06-2.90 | 82.97-1.91 |
| | (30.8-2.90) | (20.1-1.91) |
| R_{sym} or R_{merge} | 0.075 (0.640) | 0.073 (0.494) |
| I/σI | 11.6 (1.9) | 8.5 (2.5) |
| Completeness (%) | 98.8 (99.7) | 90.5 (88.7) |
| Redundancy | 4.0 (4.3) | 2.8 (2.9) |
| CC½ | 0.998 (0.663) | 0.997 (0.877) |
| Refinement | | |
| Resolution (Å) | 45.95-2.90 | 82.97-1.91 |
| No. reflections (total/test) | 27587/1335 | 161613/8036 |
| R_{work}/R_{free} | 25.6/29.5% | 20.5/24.4% |
| No. atoms | | |
| Protein | 7597 | 16444 |
| Water | — | 708 |
| B-factors | | |
| Protein | 79.0 | 41.7 |
| Water | — | 42.4 |
| R.m.s. deviations | | |
| Bond lengths (Å) | 0.008 | 0.10 |
| Bond angles (°) | 1.05 | 1.19 |

*Values in parentheses are for highest-resolution shell.

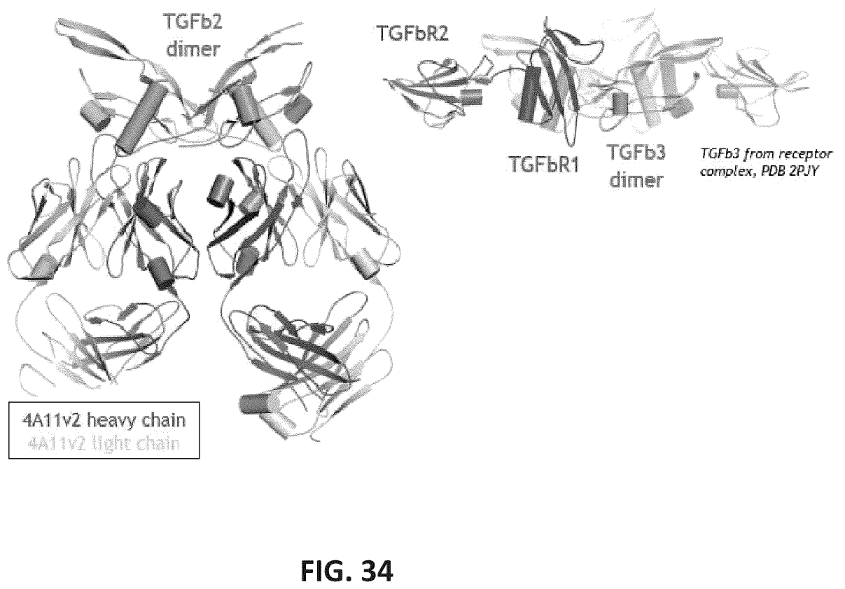

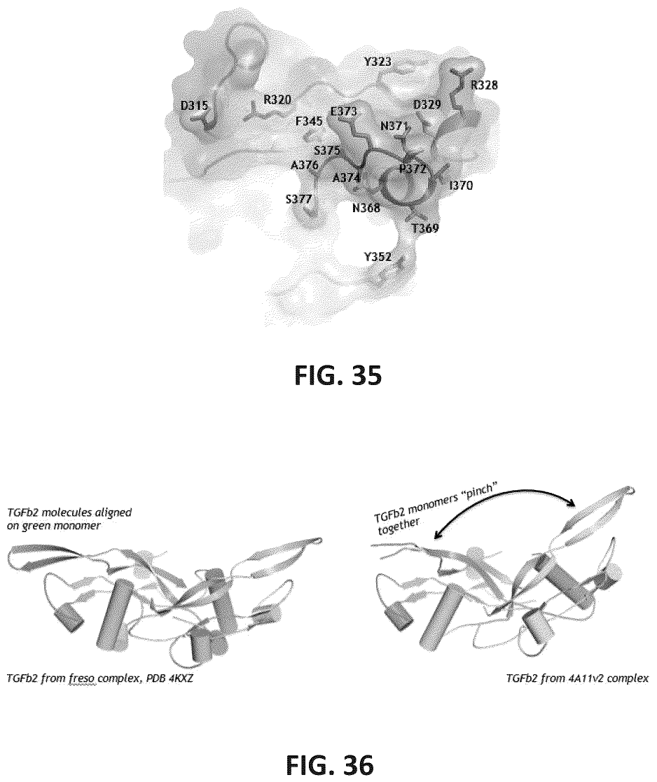

The epitope for 4A11 is found on the underside of the TGFβ32 dimer, with the two Fabs bound to two identical epitopes as expected for a symmetric homodimer (FIG. 34). The epitope for 4A11 encompasses TGFβ32 residues V313, Q314, D315, R320, L322, Y323, R328, D329, F345, and A347 in one TGFβ32 monomer, and N368, T369, I370, N37I, P372, E373, A374, S375, A376, and S377 in the second TGFβ32 monomer (FIG. 35 and Tables 28 and 29). For the 4A11v2/TGFβ32 structure, ~1760 Å2 of surface area is buried, and for 4A11v7/TGFβ32 structure, ~1950 Å2 of surface area is buried. The 4A11 epitope is centered on the cap of an alpha helix (residue 359-372). Within the epitope, there is significant conservation of sequence between TGFβ1/2/3, but one major difference found in TGFβ1 but not TGFβ32/3 is a change from glutamic acid to glycine at position 373 (human TGFβ32 numbering) (FIG. 35). This glutamic acid residue lies at the center of the 4A11 epitope, and makes a polar contact with CDR-H1 T33. A change from glutamic acid to glycine would disrupt this polar interaction as well as remove a large amount of sidechain introduced surface area.

TABLE 28
Contact Residues on TGFβ2 chain A and 4A11 Mab

| TGFβ2 | 4A11 | 4A11 |
|---|---|---|
| chain A | Light Chain | Heavy Chain |
| V313 | | S28 |
| | | S30 |
| | | S31 |
| Q314 | | S28 |
| | | S30 |
| D315 | | S28 |
| | | L29 |
| | | S30 |
| | | S73 |
| R320 | | S30 |
| P321 | | Y53 |
| | | G102 |
| | | G103 |
| L322 | | Y53 |
| | | G103 |
| Y323 | | G103 |
| | | A104 |
| | | P105 |
| R328 | Y30 | P105 |
| | N31 | |
| D329 | Y30 | |
| F345 | | Y53 |
| | | G54 |
| A347 | | G54 |

TABLE 29
Contact Residues on TGFβ2 chain B and 4A11 Mab

| TGFβ2 chain B | Light Chain | Heavy Chain |
|---|---|---|
| N368 | G96 | S56 |
| | K100 | Y58 |
| T369 | S95 | Y58 |
| | G96 | |
| | S97 | |
| | K100 | |
| I370 | Y30 | |
| | Y94 | |
| | S95 | |
| | G96 | |
| | K100 | |
| N371 | Y30 | Y50 |
| | Y94 | |
| | S95 | |
| | K100 | |
| P372 | A30 | Y50 |
| | A34 | |
| | G93 | |
| | Y94 | |
| | S95 | |
| | K100 | |
| | Y101 | |
| E373 | Y101 | T33 |
| | | Y50 |
| | | Y53 |
| | | H98 |
| | | Q100 |
| | | V101 |
| | | G102 |
| | | G103 |
| | | G107 |
| | | S108 |
| A374 | K100 | S52 |
| | | Y53 |
| | | G54 |
| | | S56 |
| | | Y58 |
| | | S52 |
| S375 | | Y53 |
| | | G54 |
| | | G55 |
| | | S56 |
| | | G54 |
| A376 | | G55 |
| | | S56 |
| | | G55 |
| S377 | | S56 |

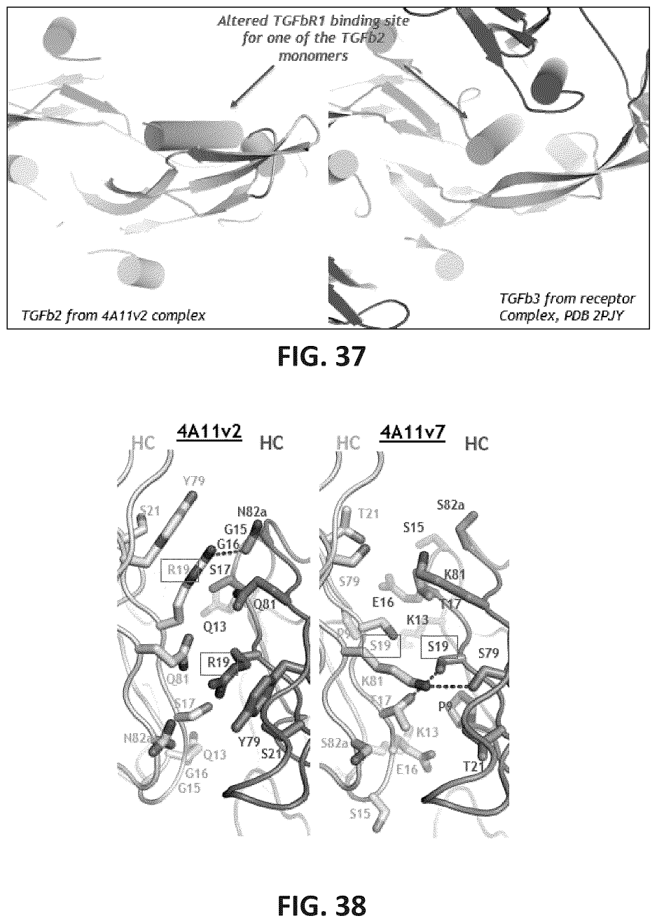

As shown in FIG. 34, the 4A11 epitope is away from the TGFBR1/TGFBR2 binding sites, and neither 4A11 molecule sterically competes for receptor binding. Instead, the 4A11 Fabs appear to induce a conformational change in TGFβ2, causing the two monomers to “pinch” together by several degrees (FIG. 36). Comparison of the 4A11 bound TGFβ2 to TGFβ3 bound to TGFBR1 and TGFBR2 suggests that the receptor binding site for at least one of the TGFβ2 monomers is compromised by this conformational change (FIG. 37). These changes apparent in the crystal structure indicate that 4A11 uses an allosteric mechanism to inhibit TGFβ2/3 signaling, by conformational disruption of one or both of the receptor binding sites. Importantly, bivalent binding of a 4A11 IgG molecule would be required to induce this conformational change in TGFβ2/3, and therefore inhibit TGFβ2/3 signaling. Unlike a receptor blocking antibody, where blocking one R1/R2 receptor binding site on the TGFβ dimer can block signaling, single arm binding of 4A11 is unlikely to be sufficient to inhibit signaling. Given the location of the epitopes, divalent binding of a full 4A11 IgG molecule may also sterically prevent an IgG bound TGFβ2/3 molecule from getting close enough to the membrane to any of the TGFB receptors.

During the humanization process for 4A11, it was noted that a number of antibody framework sequences caused a disconnect between antibody affinity and IC₅₀ in a cell based assay. For example, 4A11v2 had picomolar affinity for TGFβ2, but lost all activity in cell based assays, while 4A11v7 had similar in vitro affinity to v2, but retained very potent activity in cell based assays. As previously mentioned, V2 and v7 were humanized to the VL1VH3 and VL3VH4 frameworks respectively, which have significant sequence differences. It was therefore investigated more closely how framework residues may influence the ability of 4A11 to form a divalent complex with the TGFβ2 dimer (FIG. 38).

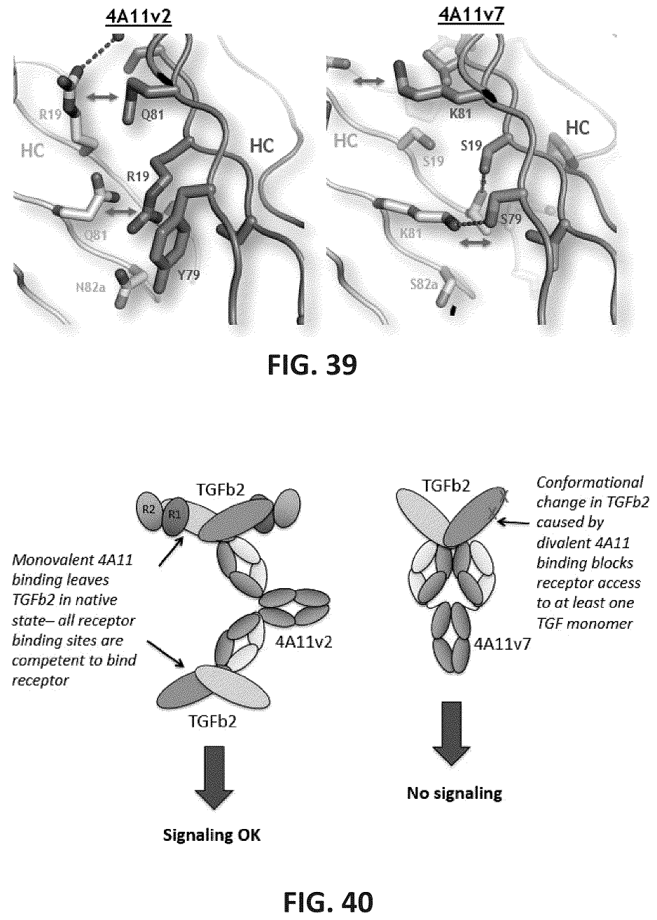

The structures of 4A11v2 and v7 with TGFβ2 show that the heavy chain frameworks come close together in the structure, and actually do interact to form an interface. The residues within those framework regions could therefore have an effect on the ability of a 4A11 IgG molecule to form a divalent complex. A close examination of this HC/HC framework interface suggests that the v2 framework residues are a less permissive interface than that seen for v7 (FIG. 39). For example, HC position 19 is an Arginine in v2 and a serine in v7, and HC position 81 is a Glutamine in v2 and a Lysine in v7. In v2, the R19 and Q81 push close together, forming what looks to be an entropically unfavorable contact, with no stabilizing polar interaction. In v7, the S19 residue makes space for K81 to reach out and form a H-bond with Ser79. In addition, v2 has two large bulky residues (Y79 and N82a) that do not form obvious productive contacts. Importantly, these positions are smaller residues in v7 (S79 and S82a), which likely allows the HC framework interface to come together in a more permissive fashion. While these HC framework contacts in v7 do not appear to be particularly complementary, they do seem to be more permissive (less unfavorable) than the framework contacts between the two v2 heavy chains. These interactions appear to be unfavorable, due to both steric bulkiness and potential entropic penalty of burying large flexible sidechains.

Further corroborating the above analysis, calculation of the buried surface area for the HC/HC framework interactions in the v2 and v7 structures suggest the v7 HC/HC contact is more complementary. In the 4A11v2/TGFβ2 structure, the HC/HC interface (chain D and chain F) buries approximately 660 Å2 of surface area. For the 4A11v7/TGFβ2 structure, there are two of the 2:2 complexes in the asymmetric unit of the crystal, and between the two sets of HC/HC framework interactions, 1050 Å2 (chain D/F) and 730 Å2 (chain B/H) are buried. The large difference here between the two 2:2 complexes is likely caused by crystal packing artifacts, but the overall trend suggests the v7 HC/HC interface is larger, suggesting a more complementary contact area. It is also important to mention that in the context of a crystallization experiment, the protein concentrations are very high (>10 mg/mL) and at those concentrations repulsive effects of non-complementary HC/HC framework interactions can be overcome and the divalent complex formed. In the context of a lower-concentration solution-based experiment (such as cell based assay, or in vivo) repulsive effects induced by the framework could cause monovalent binding to be preferred over divalent binding.

The implications of these framework differences are important to explain the differences in cell-based activity of 4A11v2 vs v7 (FIG. 40). If at a low concentration, v2 cannot divalently bind to the TGFβ2/3 dimer, this antibody will not induce the necessary conformational change in TGFβ to block the second receptor site and inhibit signaling. For v7, the framework contacts are permissive for divalent binding to TGFβ2/3 dimer, and therefore this antibody can induce the conformational change that blocks receptor tetramer formation, and therefore can inhibit signaling. This data all points to a novel mechanism of allosteric inhibition of TGFβ2/3 by the 4A11 antibody, that does not rely on steric block of (interference with) the receptor, but instead requires divalent binding to the TGFβ dimer to block signaling.

### Example 11: Safety Comparison to Pan TGFβ 1D11 Antibody

In this example, 2A10 mAb, an antibody having the 2A10.v4 variable region with a murine IgG1 Fc region, or the pan-TGFβ antibody 1D11, is administered thrice weekly (TIW) for 4 weeks (12 total doses) to CD-1 mice at doses of 0 (control), 10 and 50 mg/kg I.P. TIW, and 50 mg/kg I.V. TIW in a vehicle consisting of phosphate buffered saline. Males and females are assigned to the toxicity groups at all dose levels and are scheduled for necropsy at the end of the dosing period (Day 29). Blood for toxicokinetic evaluation is collected from additional mice in the control and 2A10/2A10v4 mAb treated groups (n=9 males/group). Criteria for evaluations include the following parameters: clinical observations, body weight, clinical pathology (hematology and clinical chemistry), anatomic pathology, and toxicokinetics.

The AUC exposures are determined, and expected to increase approximately dose-proportionally with an increase in dose from 10 to 50 mg/kg.

No 2A10- or 2A10.v4-related clinical observations, effects on body weight, or clinical pathology changes are expected, whereas such related clinical observations, effects on body weight, or clinical pathology changes indicating increased toxicity are expected for mice treated with 1D11 antibody, e.g., histologic lesions, weight loss, nonneoplastic cystic epithelial hyperplasia and inflammation of the tongue and dental dysplasia and epithelial hyperplasia of the gingiva and esophagus, as described in Lonning et al. (2011) Current Pharmaceutical Biotechnology, 12, 2176-2189.

### Example 12: Safety Comparison to Anti-TGFβ 1 Selective Antibodies

In this example, 2A10 mAb, an antibody having the 2A10.v4 variable region fused to a murine IgG1 Fc region, the variable region of the anti-TGFβ1 antibody CAT-192 (metelimumab) fused to murine IgG1 Fc, are each administered thrice weekly (TIW) for 4 weeks (12 total doses) to CD-1 mice at doses of 0 (control), 10 and 50 mg/kg I.P. TIW, and 50 mg/kg I.V. TIW in a vehicle consisting of phosphate buffered saline. Males and females are assigned to the toxicity groups at all dose levels and are scheduled for necropsy at the end of the dosing period (Day 29). Blood for toxicokinetic evaluation is collected from additional mice in the control and 2A10/2A10v4 mAb treated groups (n=9 males/group). Criteria for evaluations include the following parameters: clinical observations, body weight, clinical pathology (hematology and clinical chemistry), anatomic pathology, and toxicokinetics.

The AUC exposures are determined, and expected to increase approximately dose-proportionally with an increase in dose from 10 to 50 mg/kg.

No 2A10- or 2A10.v4-related clinical observations, effects on body weight, or clinical pathology changes are expected, whereas such related clinical observations, effects on body weight, or clinical pathology changes indicating increased toxicity are expected for mice treated with CAT-192, e.g., as described in Denton A&R 56:323 (2007).

### Example 13: Full-Length TGFβ3 Conditioned Media Inhibits Alveolosphere Formation In Vitro

### Materials and Methods

### Lung Cell Isolation

Lung cells were isolated following a previously published protocol (see, Rock et al., 2011 Proceedings of the National Academy of Sciences of the United States of America 108, E1475-1483). Tissues were disrupted and single cells were collected after lungs were inflated and digested with a protease solution cocktail (5 U/ml Dispase, 450 U/ml Collagenase Type I, 4 U/ml Elastase and 0.33 U/ml DNaseI in DMEM/F12) for 45 minutes at 37 °C with frequent agitation. Cells were then washed by DMEM with 10% Fetal Bovine Serum (FBS, Invitrogen) and resuspended in ACK lysis buffer for 3 minutes to lyse red blood cells. After washing, cells were blocked by purified rat anti-mouse CD16/CD32 antibody (Cat #553142; BD Biosciences) and stained with antibodies for CD45 (Cat #17-0451-82, eBioscience), EpCAM (Cat #11-5791-82, eBioscience). AEC2 cells were sorted on a BD FACSAria Fusion cell sorter (BD Biosciences), based on the following markers: CD45⁻; EpCAM⁺; and tdTomato⁺. Data was analyzed by Flowjo software.

### AEC 3-D Organoid Culture

AEC organoid culture was performed by following a previously published protocol with minor modifications (see, Barkauskas, 2013 JCI 123, 3025-3036; Sun T, et al, 2019, JCI Insight; June 18; 5(14):e128674). Sorted AEC2 cells were mixed with human stromal cell NHLF (Cat #CC-2512; Lonza Walkersville Inc. USA) at a 1:10 ratio. The cells were resuspended in pre-chilled 1:1 mixed media of MTEC/Plus and growth factor-reduced Matrigel (Cat #356231; BD Biosciences). 10⁴ AEC2 cells and 10⁵ NHLF cells were placed in 90 μl mixed media in a 24-well 0.4-μm transwell insert (Cat #3470; Costar). 500 μl MTEC/Plus media was added to the lower chamber. Media were changed every other day. Organoids were assessed after 12-14 days of culture by inverted fluorescence microscope.

### Microscopy

The whole organoid culture images were captured on a Nikon Ti-E Perfect Focus inverted microscope. Large images were generated by automatically stitching multiple adjacent frames from a multipoint acquisition using a motorized stage. Images were analyzed by ImageJ software.

### Secretome Library Screening

The Secretome library is a collection of plasmids expressing individual human cDNA on mammalian expression vectors, containing ~1,700 individual full-length genes that encode secreted proteins. To identify factors that may be involved in AEC biology in the alveolar organoid system, the library was first divided into 96 sub-libraries, with each containing around 10-20 individual plasmids. Each sub-library plasmid pool was transiently transfected into 293T cells using Lipofectamine 2000 (Cat #11668019, ThermoFisher) by following the manufacturer's recommendations. Conditioned media were collected two days later, kept at 4 °C until used in the alveolosphere culture. Positive sub-libraries were identified at the end of the culture. During the second round of screening, conditioned media from individual plasmids in each positive sub-library were generated and tested. Positive single genes responsible for the organoid culture phenotypes were eventually identified.

### Results

The lung alveolar epithelium is maintained and repaired by proliferation and hyperplasia of type 2 alveolar epithelial cells (AEC2), followed by differentiation into type 1 alveolar epithelial cells (AEC1). To investigate AEC biology, a 3-D alveolosphere organoid culture system was employed, in which fate-mapped AEC2s were isolated from Sftpc-CreER^{T2}; Rosa26R-tdTm mice after tamoxifen induction and co-cultured with lung fibroblasts in Matrigel. After ~14 days, spheroids derived from individual AEC2s form, with an outer layer of AEC2s and an inner lumen with AEC1s that have differentiated from AEC2 progenitors. This system was previously employed to identify TAZ as a regulator of AEC2-AEC1 differentiation during alveolar repair (see, Sun, T. et al. (2019). JCI Insight; June 18; 5(14):e128674). To identify potential secreted factors that affect alveolar epithelial cell proliferation, a screen was performed using an expression plasmid library carrying approximately 1,700 individual human genes encoding secreted proteins. This plasmid library was transiently transfected into 293T cells, first in pools (10-20 individual plasmids/pool) and subsequently individually, harvested the conditioned media two days later, and added conditioned media to alveolosphere cultures. Conditioned media from cells transfected with a construct encoding full-length TGFβ3 completely inhibited alveolosphere formation. In contrast, conditioned media from cells transfected with full-length TGFβ1 had no effect, despite using the same transfection conditions and expression vectors. Prior studies have shown that the receptor-binding domain (“mature” form) of TGFβ1 can promote apoptosis of primary type 2 alveolar epithelial cells (AEC2) in vitro and in vivo. Indeed, recombinant mature forms of TGFβ1 or TGFβ3 inhibited alveolosphere formation in these experiments. Therefore, under the culture conditions used, full length LAP-associated TGFβ1 is inactive, but full length LAP-associated TGFβ3 is active, suggesting that “latent” TGFβ3 has intrinsic activity and/or it can be activated by a unique mechanism presented in this culture system, distinct from those described for TGFβ1.

### Example 14: Full-Length TGFβ2 and TGFβ3 can Activate TGFβR-Dependent Signaling Independent of Integrin Binding In Vitro

As integrin binding is an established mechanism of TGFβ1 activation, and differential activity of TGFβ1 and TGFβ3 was observed in the alveolosphere model, it was next sought to determine the integrin dependence of TGFβ3 activation. A 2-cell in vitro reporter system was used, in which expression vectors encoding full-length TGFβ1 or TGFβ3 were cotransfected into 293T cells with expression vectors encoding av and β6 integrins, and co-cultured with a reporter cell line driving luciferase expression downstream of a SMAD-dependent promoter element (“MLEC”) (see, Abe et al., 1994; Anal Biochem, 216, 276-284). While full-length TGFβ1 required co-expression of αvβ6 integrin to induce reporter activity, it was found that full-length TGFβ3 robustly induced reporter activity without co-transfection of αv and β6 integrin constructs. Co-expression of αvβ6 further increased full-length TGFβ3 dependent reporter activity, and this further increase was attenuated by mutation of the RGD integrin-binding motif in the LAP of TGFβ3 to RGE (FIG. 41).

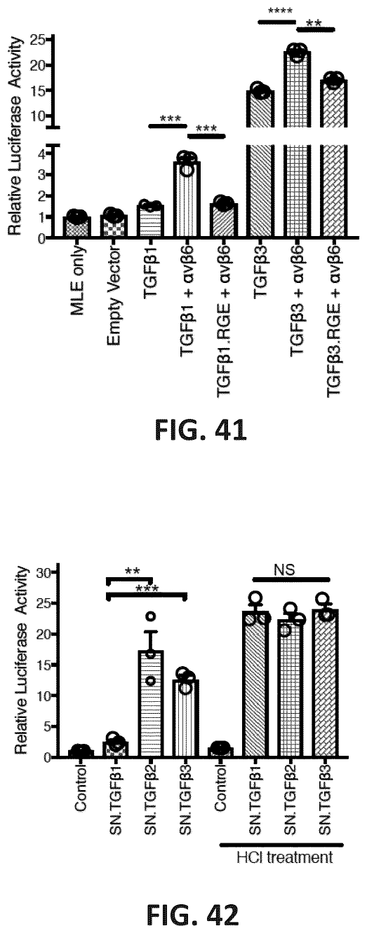

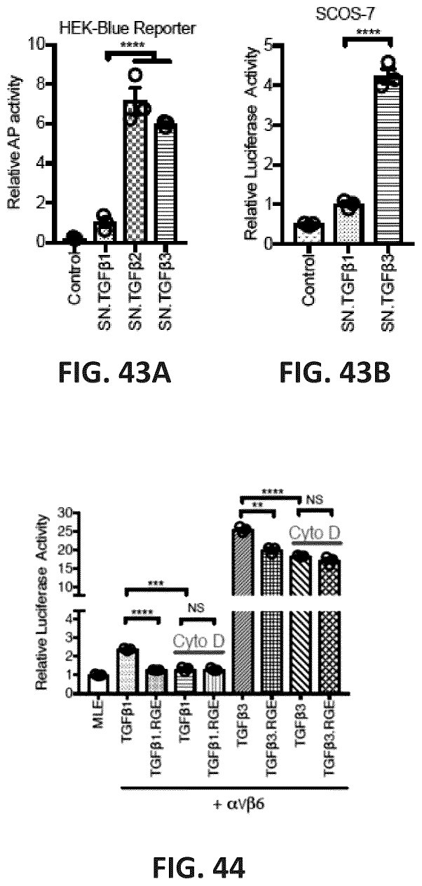

Consistent with the observed effects from the alveolosphere experiment described above, supernatants from cells transfected with full-length TGFβ3, but not TGFβ1, robustly induced reporter activity (FIG. 42). Similar reporter induction was observed with supernatants from full-length TGFβ2-transfected cells; as TGFβ2 lacks an RGD motif (FIG. 1), its activation is likely integrin-independent (FIG. 42). Total TGFβ activities from these three supernatants were comparable after an acidification step, which releases the receptor-binding domain of TGFβ from its latent complex (FIG. 42). To rule out the possibility that these effects were specific to the reporter cell lines we used, HEK-Blue (InvivoGen) and SCOS-7 cells were assessed as alternative TGFβ reporter and transfection recipient cell lines, respectively. High “intrinsic” activities of TGFβ2 and/or TGFβ3 were observed in both cases (FIG. 43A and FIG. 43B).

Cytoskeletal tension is required to transduce forces during integrin αvβ6-mediated activation of latent TGFβ1. To discount the possible involvement of other cytoskeleton dependent integrin activities, this culture system was treated with cytochalasin D to inhibit actin polymerization, which reduced the integrin αvβ6-mediated activation of TGFβ1 and TGFβ3. However, the “intrinsic” activity of TGFβ3 remained unaffected (FIG. 44), suggesting that this activity is integrin independent.

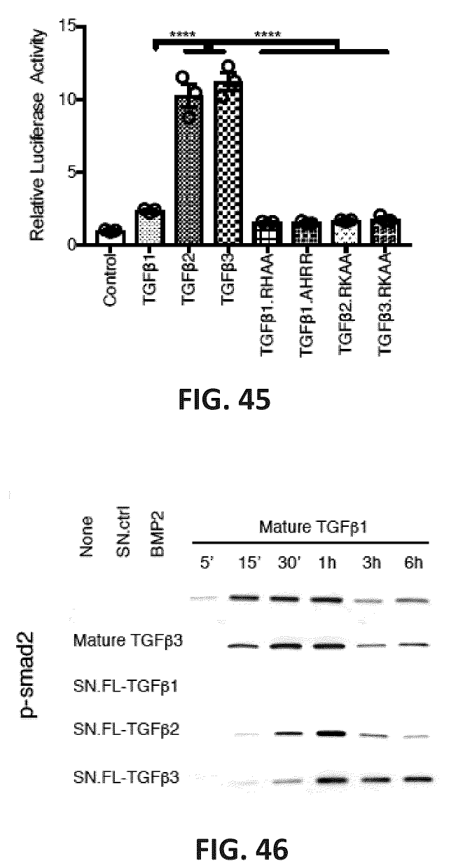

In the Golgi apparatus, the pro-peptide of TGFβ1 (pro-TGFβ1) is proteolytically cleaved by furin, leading to the formation of the SLC. This proteolytic digestion is essential to generate functional TGFβ1. All three TGFβ isoforms have conserved furin cleavage sites (RKKR or RHRR, FIG. 1). To assess the requirement for this protease cleavage for TGFβ2/3 activity in our system, the furin cleavage sites in each TGFβ isoform were mutated. Mutated full-length TGFβ2 and β completely lost their intrinsic activities (FIG. 45). Thus, like TGFβ1, furin cleavage is required for TGFβ2/3 activity, despite distinct extracellular activation mechanisms of the SLCs. To evaluate the kinetics of “intrinsic” TGFβ2 and TGFβ3 activity, phospho-SMAD2 was assessed in normal human lung fibroblasts (NHLF) after treatment with transfection supernatants and observed similar kinetics to cells treated with recombinant TGFβ receptor-binding domains, with pSMAD2 peaking about 1 h after stimulation (FIG. 46).

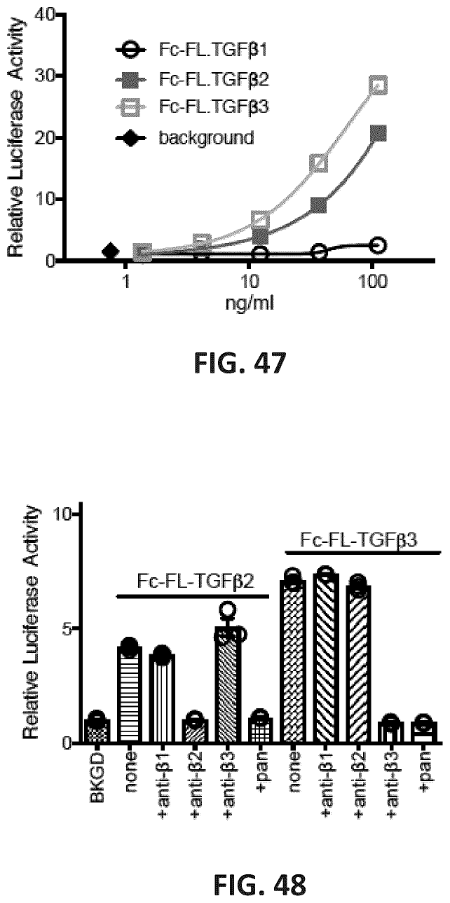

To discount the possibility that other factors present in the conditioned media of transfected cells could contribute to TGFβ2 and TGFβ3 activation, recombinant full-length TGFβ isoforms were generated. To improve expression and stability and to facilitate purification, in-frame N-terminal fusions were made of the constant domain of human IgG1 (“Fc”) to each isoform (Fc-FL-TGFβ1, 2, and 3). Consistent with the effects observed in conditioned media, purified recombinant Fc-FL-TGFβ1 did not activate reporter activity in MLEC. However, Fc-FL-TGFβ2 and Fc-FL-TGFβ3 robustly induced reporter activities (FIG. 47), which were inhibited by their respective isoform-specific antibodies (FIG. 48).

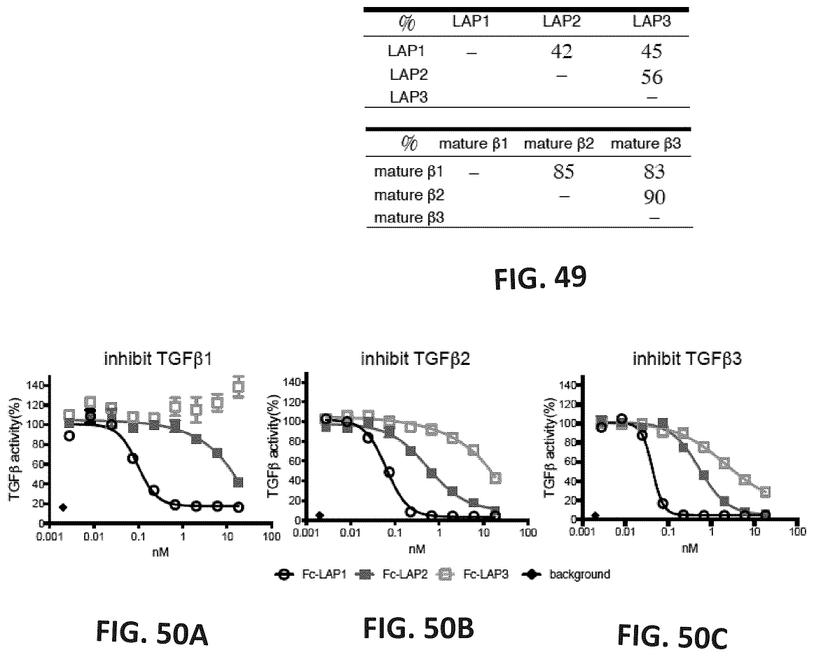

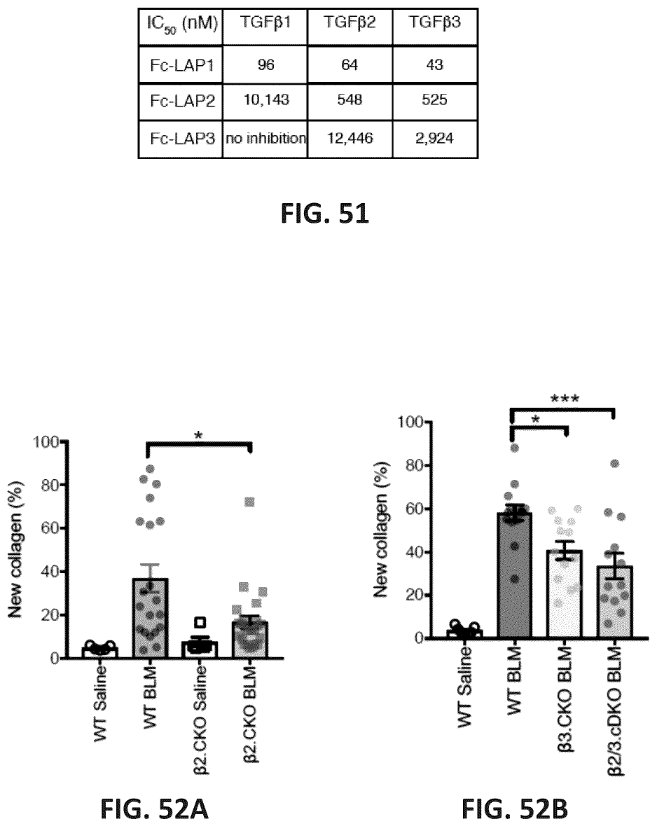

Previous studies showed that recombinant LAP from TGFβ1 (LAP1) can strongly inhibit the receptor-binding domains of TGFβ1, 2, and 3 in vitro and in vivo, presumably by reconstituting them into SLCs. While the receptor-binding domains of TGFβ isoforms are highly homologous, the LAP domains are more diverse (FIG. 49). Thus, it was postulated that the LAPs from TGFβ1, 2, and 3 (LAP1, 2 and 3, respectively) might have differential inhibitory capacities. Recombinant N-terminal fusion proteins of human IgG1 with human LAP1, 2, and 3 (Fc-LAP1, 2, and 3) were generated. Substantially different neutralization capabilities were observed for each Fc-LAP. Consistent with observations with conditioned media and recombinant full-length TGFβ proteins, Fc-LAP1 more potently inhibited the receptor-binding domains of all three TGFβ isoforms than Fc-LAP2 or Fc-LAP3 (FIGS. 50A, 50B and 50C and FIG. 51). Taken together, these data show that the relative potencies of LAP1, 2, and 3 to inhibit TGFβ activity are divergent and may explain the “intrinsic” activities of full-length TGFβ2 and TGFβ3 not observed for TGFβ1.

Taken together, these data show that FL-TGFβ2 and TGFβ3 may have distinct activation mechanisms and thresholds from those of TGFβ1. Although FL-TGFβ1 can be activated by ectopic expression of αvβ6 integrin, and the activity of FL-TGFβ3 can be increased by αvβ6 integrin binding, both FL-TGFβ2 and FL-TGFβ3 appear to have “intrinsic” activities, and/or are activated by as-yet unidentified mechanisms. The biochemical inhibitory potencies of LAP1, 2, and 3 are divergent, with the biochemical IC50 of LAP1 against each receptor-binding isoform being several orders of magnitude lower than those of LAP2 and LAP3. Given that latent TGFβ1 has a much higher threshold for activation than latent TGFβ2 or TGFβ3, these mechanistic observations suggest that TGFβ2 and TGFβ3 gene expression are more likely than TGFβ1 gene expression to reflect their activities in tissue.

The receptor-binding domains of TGFβ1, 2, and 3 are highly homologous, with 83-90% amino acid similarity. Dimeric receptor-binding domains of all three isoforms bind to pairs of heterodimeric receptor complexes of TGFβR1 and TGFβR2; the tetrameric receptor complex then activates intracellular signaling via receptor tyrosine kinase (RTK) activity of TGFβR1, also known as ALK5. In canonical TGFβR signaling, ALK5 phosphorylates SMAD2 and SMAD3, which then associate with SMAD4, translocate to the nucleus, and direct gene transcription. Additional non-SMAD dependent signaling pathways such as MAP kinases, AKT, JAK-STAT, and NFκB may be activated downstream of TGFβRs in certain contexts (Derynck, 2019 Science signaling 12). Biochemical and structural studies have shown that the assembly of TGFβ-TGFβR signaling complexes have subtle isoform-specific differences: TGFβ1 and TGFβ3 bind more strongly to TGFβR2 and only form strong interactions with TGFβR1 when in complex with TGFβR2, whereas TGFβ2 binds weakly to both TGFβR1 and TGFβR2 alone, and avidity may drive full complex formation (Radaev, 2010, JBC 285, 14806-14814). TGFβ1 and TGFβ2 crystallize in “closed” structures that facilitate binding to TGFβR1 and TGFβR2, while TGFβ3 can adopt a similar “closed” or less ordered “open” structure in crystal form, which may lead to differences in the avidity of ligand-receptor complex assembly. In addition, TGFβR3 (betaglycan), a non-signaling receptor, can facilitate TGFβ2 binding to TGFβR1/2 complexes, but does not appear to play a similar role in TGFβ1 or TGFβ3 receptor binding (del Re, 2004 JBC 279, 22765-22772). The structural analyses of the allosteric inhibitory mechanism of 4A11 demonstrated herein suggest that bivalent antibody binding to a TGFβ2 or TGFβ3 dimer constrains TGFβ in a form that precludes simultaneous binding to two sets of receptor heterodimers (FIG. 7). Ultimately, despite these differences in signaling complex assembly, recombinant receptor-binding domains of TGFβ1, 2, and 3 are all able to induce TGF PR-dependent SMAD signaling to a similar degree in cell-based in vitro assays. Hence, any differences in the in vivo activity of endogenous TGFβ isoforms are more likely due to differences in their patterns of expression and mechanisms of release from SLC or LLC than to differences in their receptor-binding domains.

### DISCUSSION

The LAP domains of TGFβ1, 2, and 3 are substantially less homologous than the receptor-binding domains, with only 42-56% amino acid similarity. All three isoforms have conserved furin cleavage sites (RKKR or RHRR) between the LAP and receptor-binding domains, and a conserved “fastener” sequence (YYAKE). TGFβ1 and TGFβ3 have an RGD integrin-binding sequence but TGFβ2 has an R-S substitution at that site and has not been shown to be activated via integrin binding. Structures of the SLC including the LAP and receptor-binding domain have been solved for TGFβ1 but not for TGFβ2 or 3. The “finger” region of the receptor-binding domain of TGFβ1 that interacts with TGFβR2 is shielded by a “latency lasso”, a loop between the α1 and α2 helices of the LAP that is associated with the fastener sequence. In all three isoforms, the C-terminal sequence of the LAP α1 helix is highly conserved, with a lysine residue (GQILSKL) that interacts with two conserved tyrosines (YYAKE) in the fastener of TGFβ1; in addition, the alanine (YYAKE) interacts with an arginine in the α5 helix near the C-terminus of the LAP that is conserved across the three isoforms (Shi et al. 2011; Nature 474, 343-349).

The latency lassos of the three TGFβ LAP isoforms show some sequence diversity, with 6 prolines in TGFβ1, 5 in TGFβ2, and 4 in TGFβ3, and the length of the lasso is 1 and 2 residues shorter in TGFβ2 and TGFβ3, respectively, than in TGFβ1. There are substantial sequence differences between the three isoforms around the RGD integrin binding site: TGFβ2 has an SGD sequence, does not likely bind integrins, and is 9 residues longer than TGFβ1 between the β9 and β10 strands; TGFβ3 has an RGD and is 3 residues longer than TGFβ1 between the β9 and β10 strands; and the loops between the β4 and β5 strands and β7 and β8 strands are substantially longer in TGFβ2 and TGFβ3 than they are in TGFβ1. While structure-function relationships have not yet been elucidated for these regions, it is possible that they may contribute to different modes of activation and/or inhibitory potency of the LAP domains of TGFβ2 and TGFβ3 as compared to TGFβ1. Heterozygous loss-of-function mutations have been described in TGFB2 and TGFB3 genes associated with Loeys-Dietz syndrome (see, Lindsay, 2012 Nature Genetics; 44, 922-927; Bertoli-Avella, 2015 J Am Coll Cardiol 65, 1324-1336; Schepers, 2018 Human Mutation 39, 621-634) in key residues in the LAP domains, implicating the furin cleavage site in both, the fastener in TGFβ2, and the RGD integrin-binding site in TGFβ3, suggesting that these conserved motifs are important for at least some functions of TGFβ2 and TGFβ3. While it is shown herein that full-length TGFβ2 and TGFβ3 carry “intrinsic” activity, this activity remains dependent on furin cleavage, and in the case of TGFβ3, can be augmented by integrin binding. Consistent with the notion that the LAP, rather than the receptor-binding domain, confers activation specificity, it is shown herein that the LAP of TGFβ1 can potently inhibit all three isoforms, whereas the LAPs of TGFβ2 and TGFβ3 are significantly less inhibitory.

### Example 15: Pharmacokinetics of Anti-TGFβ Isoform-Specific Antibodies in C57BL/6 Mice

Pharmacokinetics (PK) of three chimeric antibodies (6F2, 2A10, and 4A11) were characterized following a single intravenous (IV) dose of 1 or 10 mg/kg, or intraperitoneal (IP) dose of 10 mg/kg in female C57BL/6 mice. All three antibodies demonstrated favorable PK profiles, with apparent biphasic distribution characterized by a short distribution phase followed by a longer elimination phase. Linear and dose-proportional (Cₘₐₓ and AUC; area under the concentration-time curve) PK were observed between 1 and 10 mg/kg, with clearance ranging from 3.5 to 4.9 mL/day/kg in all cohorts. Following single IP administration of 6F2 and 4A11 at 10 mg/kg, the AUCₗₐₛₜ was ≥97% and Cₘₐₓ was ~50-60% when comparing the AUCₗₐₛₜ and Cₘₐₓ after IV administration at 10 mg/kg. The data are shown in the Table 30, below.

TABLE 30
PK of anti-TGFβ isoform-specific antibodies in C57BL/6 mice

| | | | Dose | Cₘₐₓ | AUCₗₐₛₜ | CL | T_{1/2,λz} |
|---|---|---|---|---|---|---|---|
| Antibodies | Specificity | Route | (mg/kg) | (μg/mL) | (day · μg/mL) | (mL/day/kg) | (day) |
| 6F12 | TGFβ2 | IV | 1 | 23.3 | 104 | 4.89 | 10.2 |
| | | IV | 10 | 273 | 1163 | 3.88 | 15.5 |
| | | IP | 10 | 134 | 1197 | 3.58 | 17.5 |
| 2A10 | TGFβ3 | IV | 10 | 305 | 1910 | 3.56 | 13.1 |
| 4A11 | TGFβ2/3 | IV | 1 | 18.1 | 118 | 4.58 | 10.6 |
| | | IV | 10 | 233 | 1486 | 4.16 | 9.2 |
| | | IP | 10 | 133 | 1456 | 4.39 | 5.5 |

Cₘₐₓ = maximum observed concentration; AUCₗₐₛₜ = area under the serum concentration versus time curve from time = 0 to time of the last measurable concentration; CL = clearance; T_{1/2,λz} = terminal half-life

### Example 16: In Vivo Activity of Isoform-Selective Anti-TGFβ2 and TGFβ3 Antibodies in a Lung Fibrosis Model

### Materials and Methods

### Lung Genomic DNA Isolation and Quantitative PCR

Genomic DNA was isolated from mouse whole lungs using the DNeasy Blood & Tissue Kit (Cat #69506; Qiagen). TGFβ DNA levels were quantified by qPCR using Taqman Copy Number Assays (Applied Biosystems). Threshold cycle values (Ct) were normalized to an internal reference, Tert (ΔCT). The genomic DNA levels were calculated by the 2-ΔΔCT method. The following Taqman assays from Applied Biosystems were used:
Tgfb2 Exon3 (Mm00035180_cn)
Tgfb2 Exon6 (Mm00035822_cn)
Tgfb3 Exon3 (Mm00381025_cn)
Tgfb3 Exon6 (Mm00381036_cn)
and Tert (4458373).
   Histological Analysis of Mouse Lung Fibrosis in the Bleomycin Induced Model Formalin fixed samples of mouse lungs (5 lobes) were embedded as a whole and processed to 1 slide per animal, stained with H&E or Masson's Trichrome. Alternatively, fibrosis was evaluated by immunohistochemistry using anti-collagen III antibody (Cat #ab7778, Abcam). The extent of pulmonary fibrosis was scored according to the following criteria:
(A) Interstitial fibrosis pattern—number of foci: 0, none detected; 1, ≤10; 2, ≤15; 3, >15/all sections, but distinct; 4, multifocally coalescent or locally extensive; 5, diffused
(B) Interstitial fibrosis—size of foci: 0, none detected; 1, largest focus ≤area of ~2 alveolar spaces; 2, largest focus ≤area of ~4 alveolar spaces; 3, coalescent (>4 patent alveolar spaces); 4, locally extensive (60-90% of an entire lobe); 5, diffuse (>90% of an entire lobe);
(C) Total scores: number of foci×size of foci

### Results

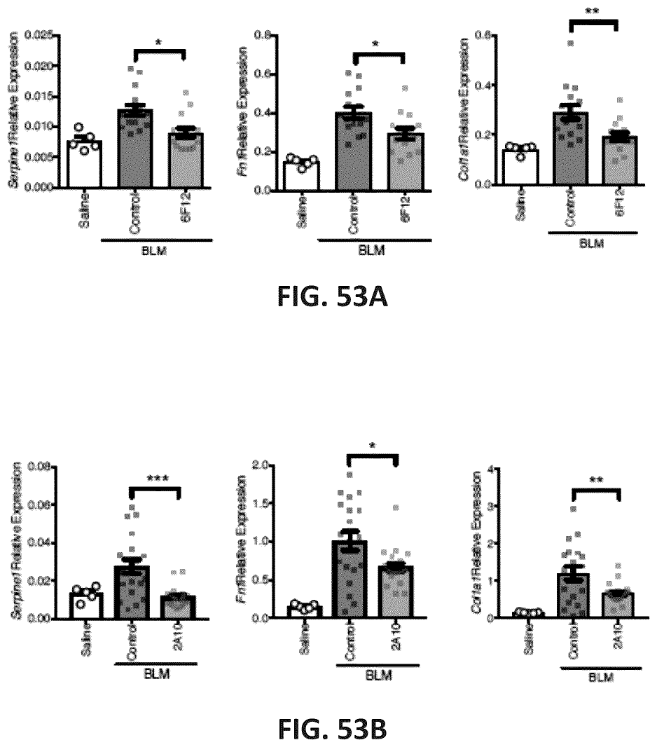

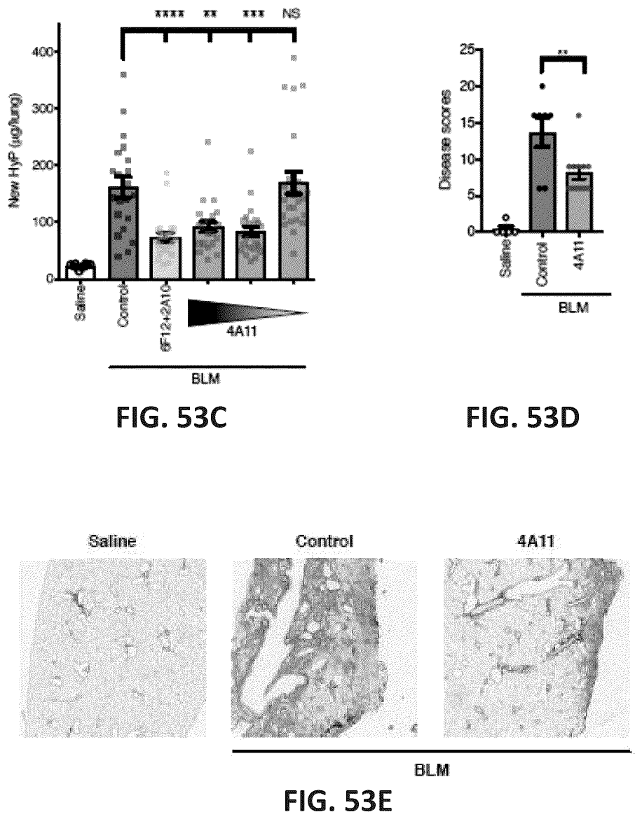

To assess the in vivo anti-fibrogenic effects of anti-TGFβ2 (6F12) and anti-TGFβ3(2A10) antibodies, mice were treated preventively and examined for TGFβ induced gene expression in lungs at day 14 after bleomycin administration, when the expression of those genes peaked. Serpine1, Fn1, and Col1a1 expression were all significantly reduced in mice treated with either antibody (FIGS. 53A and 53B). The antibody 4A11 potently inhibited the activities of both TGFβ2 and TGFβ3 isoforms in vitro (Table 30, above). In vivo, 4A11 treatment reduced new collagen accumulation at day 24 comparably to the combination of both 6F12 and 2A10 (FIG. 53C). A dose titration of 4A11 was performed and comparable efficacy at doses of 10 mg/kg thrice or once weekly, but not at 3 mg/kg/week were observed (FIG. 53C). Pathologic examination of lungs at day 24 showed significantly attenuated disease scores and collagen accumulation in the high-dose 4A11 treated group compared to the control group (FIGS. 53D and 53E). Taken together, these data show that treatment with neutralizing antibodies to TGFβ2 and TGFβ3 can reduce lung fibrosis in vivo.

### Example 17: TGFβ2 and TGFβ3 Contribute to Hepatic Fibrosis

### Materials and Methods

### CCl4 Model

8-10 week-old male C57BL/6J mice (Jackson West) were IP injected with 0.5 ml/kg body weight carbon tetrachloride (Sigma-Aldrich, #319961, 1:6 v/v diluted in corn oil) or corn oil (Sigma-Aldrich) three times per week for 6 weeks. Mice received anti-TGFb2/3 (4A11)-mIgG2a-LALAPG antibody or control (anti-gp120-mIgG2a-DANA) three times per week at 10 mg/kg for 6 weeks. The animals were terminated 72 hours after the final CCl₄ injection and whole livers and serum were collected for histological and molecular analysis.

RNA Isolation and Quantitative RT-PCR from Mouse Tissues Whole mouse lungs (bleomycin studies) or mouse liver lobes (CCl₄ studies) were isolated.

Tissues were processed using a gentleMACS Dissociator (Miltenyi Biotec) for tissue homogenization. RNA was extracted from the tissue homogenates using RNeasy Mini Kit (Cat #74106; Qiagen). Gene expression levels were quantified by RT-qPCR using Taqman RNA-to-C_{T} 1-Step Kit (Cat #4392938; Applied Biosystems). The reactions were run on a QuantStudio 6 Flex Real-Time PCR System (ThermoFisher). Threshold cycle values (Ct) were normalized to a house keeping gene, GAPDH (ΔCT). The relative gene expression levels were calculated by the 2-ΔΔCT method.

The following Taqman gene expression assay kits from Applied Biosystems were used:
Serpine (Mm00435858_m1)
Fn1 (Mm01256744_m1)
Col1a1 (Mm00801666_g1)
Col3a1 (Mm01254476_m1)
Tgfb1 (Mm01178820_m1)
Tgfb2 (Mm00436955_m1)
Tgfb3 (Mm00436960_m1)
Gapdh mouse (Cat #4352661).

### Histological Analysis of Mouse Liver Fibrosis in the CCl4 Model

Formalin fixed samples of mouse livers were processed. For each animal, one section was stained with H&E and one section was stained with Trichorme. Lesions are scored blinded to group identities for degrees of architectural disruption and extent of fibrosis based on a previous published method (Zhao et al, 2008 Pathol Int 58, 580-588).

### Results

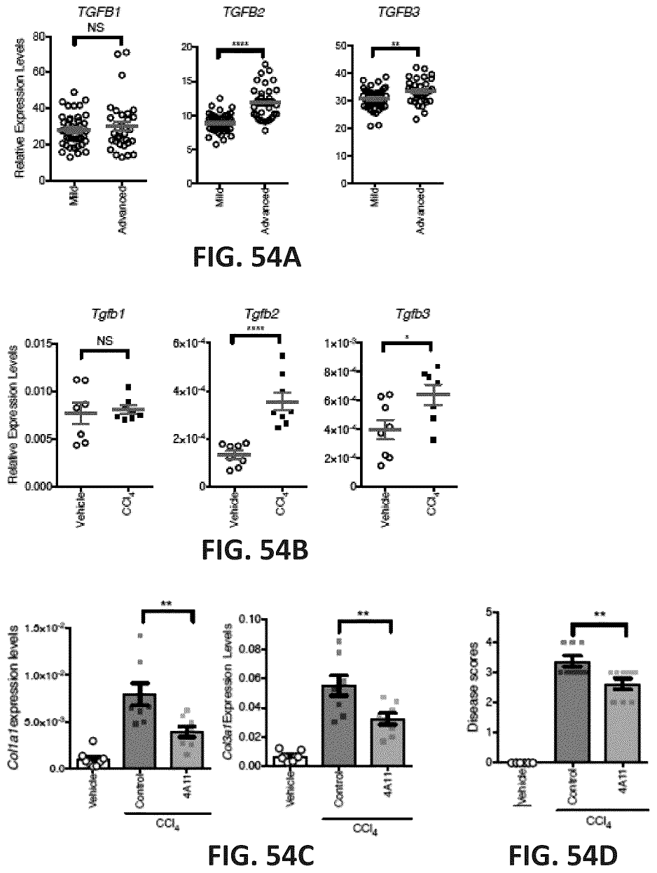

To extend the observations of Example 16, above, beyond pulmonary fibrosis, TGFβ isoform gene expression in human NAFLD/NASH patient samples were assessed from a published dataset comparing mild to advanced fibrosis (GSE49541, (Murphy et al., 2013 Gastroenterology 145, 1076-1087; Moylan et al., 2014 Hepatology 59, 471-482)). Similar to IPF lung tissue, the expression levels of TGFB1 were comparable across disease severity, while both TGFB2 and TGFB3 were significantly increased in livers from patients with more advanced fibrosis disease (FIG. 54A). In the carbon tetrachloride (CCl₄)-induced liver injury and fibrosis model in mice, upregulation of Tgfb2 and Tgfb3, but not Tgfb1 expression was observed after 6 weeks of CCl₄ challenge (FIG. 54B). Anti-TGFβ2/3 antibody (4A11) treatment significantly attenuated collagen gene expression and histological disease scores in CCl₄-challenged mice (FIGS. 54C and 54D). Histological examination showed a modest reduction of fibrosis in the 4A11 treated group when compared to controls. Taken together, these data suggest that TGFβ2 and TGFβ3 inhibition can reduce hepatic fibrosis in vivo.

### Example 18: TGFβ 2 and TGFβ3 Blockade does not Promote the Enhanced Immune Responses Associated with Pan Anti-TGFβ Blockade in a Colitis Model

### Materials and Methods

8-10-week-old C57BL/6 mice (Envigo) were inoculated orally with 0.2 ml 1×10⁹ cfu/ml Helicobacter Hepaticus suspension for two consecutive days. Mice were treated with anti-IL10R antibody (Cat #BE0050, BioXCell) weekly at 1 mg/mouse to induce colitis. To test the effects of anti-TGFβ antibodies, mice were received pan anti-TGFβ (1D11), anti-TGFβ2/3 (4A11) or control antibody three times per week at 10 mg/kg until the end of the study. The animals were euthanized at day 24, and mouse intestines were isolated, weighed and proceeded as indicated.

Mouse intestine lamina propria were isolated from cecums and proximal colons by following to the manufacturer's recommendation (Cat #130-097-410, Miltenyi). Total cell numbers were determined and cells were stained with antibodies for CD3e (Cat #553064, BD Biosciences), CD4 (Cat #553729, BD Biosciences), CD8 (Cat #553036, BD Biosciences), CD11 (Cat #553312, BD Biosciences), Ly6G (Cat #551460, BD Biosciences), Ly6C (Cat #1760-09, Southern Biotech). Data was analyzed by Flowjo software.

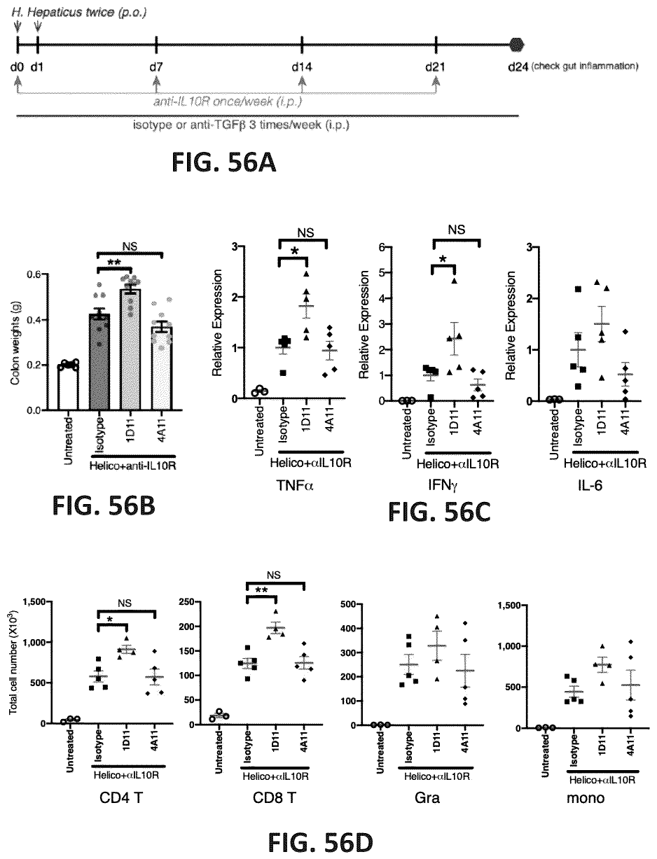

FIG. 56D provides immune cell numbers in lamina propria from the remaining mice in each group (3-5/group) as determined by flow cytometry using their surface markers: T cells (CD3+; CD4+ or CD8+); Granulocytes (“Gra,” CD11b+; Ly6G+); monocytes (“mono,” CD11b+; Ly6C+). *P<0.05, **P<0.01, NS, P>0.05 by One-way ANOVA with Dunnett's test. Data represent means±SEM. For clarity, stats bars are not shown in graphs lacking nominally significant differences between experimental groups.

### Results

TGFβ signaling plays pivotal roles in the regulation of immune responses. Previous studies with genetic or pharmacological blockade of TGFβ signaling showed excessive inflammation and leukocyte infiltration in multiple organs, resulting in serious adverse toxicities (M. J. Anderton et al., Toxicol Pathol 39, 916-924 (2011); M. S. Mitra et al., Tox Sci, 175, 24-34 (2020); J. C. Marie, et al., Immunity 25, 441-454 (2006); M. O. Li, et al., Immunity 25, 455-471 (2006). To assess the contribution of TGFβ2 and TGFβ3 blockade to systemic immune responses, Helicobacter hepaticus infected IL-10R blockade colitis model was employed (FIG. 56A). This model is often used to assess the involvement of dysregulated immune responses and intestinal microbiota, two major components of human inflammatory bowel diseases (IBD) (M. C. Kullberg et al., Infection and immunity 66, 5157-5166 (1998); M. C. Kullberg et al., Infection and immunity 69, 4232-4241 (2001); M. C. Kullberg et al., JEM 203, 2485-2494 (2006)). In agreement with previous findings, H. hepaticus infected mice treated with the anti-IL10R antibody developed colitis. Treatment with a pan-anti-TGFβ antibody (1D11) promoted increased inflammation, as evidenced by increased colon weights, inflammatory cytokine expression, and immune cell infiltration (FIGS. 56B-56D). Mice treated with the TGFβ1-sparing anti-TGFβ2/3 antibody (4A11) exhibited no increases in inflammation on those parameters. These results suggest that selective TGFβ2/3 blockade will not induce toxic inflammatory responses as observed with pan-TGβR blockade.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1.-152. (canceled)

153. An isolated anti-TGFβ2 antibody, wherein the antibody comprises:
(a) heavy chain CDRs comprising CDR-H1, CDR-H2 and CDR-H3, wherein CDR-H1 has the amino acid sequence of SEQ ID NO: 16, CDR-H2 has the amino acid sequence of SEQ ID NO: 17, and CDR-H3 has the amino acid sequence of SEQ ID NO: 18; and
(b) light chain CDRs comprising CDR-L1, -L2 and -L3, wherein CDR-L1 has the amino acid sequence of SEQ ID NO: 19; CDR-L2 has the amino acid sequence of SEQ ID NO: 20, and CDR-L3 has the amino acid sequence of SEQ ID NO: 21.

154. The anti-TGFβ2 antibody of claim 153, wherein the anti-TGFβ2 antibody selectively neutralizes TGFβ2.

155. The anti-TGFβ2 antibody of claim 154, wherein the anti-TGFβ2 antibody:
(a) has reduced toxicity relative to the pan-TGFβ antibody 1D11;
(b) has reduced toxicity in rodents relative to the pan-TGFβ antibody 1D11;
(c) has reduced toxicity relative to the pan-TGFβ small molecule inhibitor galunisertib; and/or
(d) has reduced toxicity in rodents relative to the pan-TGFβ small molecule inhibitor galunisertib.

156. The anti-TGFβ2 antibody of claim 153, wherein the anti-TGFβ2 antibody comprises a heavy chain variable region (VH) amino acid sequence having at least 95%, at least 96%, at least 97%, at least 98%, at least 99%, or 100% sequence identity to an amino acid sequence selected from the group consisting of SEQ ID NOs: 25, 132, and 138-142.

157. The anti-TGFβ2 antibody of claim 156, wherein the anti-TGFβ2 antibody comprises a heavy chain variable region (VH) amino acid sequence selected from the group consisting of SEQ ID NOs: 25, 132, and 138-142.

158. The anti-TGFβ2 antibody of claim 153, wherein the anti-TGFβ2 antibody comprises a complete heavy (H) chain amino acid sequence having at least 95%, at least 96%, at least 97%, at least 98%, at least 99%, or 100% sequence identity to the amino acid sequence selected from the group consisting of SEQ ID NOs: 31, 146, and 152-156.

159. The anti-TGFβ2 antibody of claim 158, wherein the anti-TGFβ2 antibody comprises a complete heavy (H) chain amino acid sequence selected from the group consisting of SEQ ID NOs: 31, 146, and 152-156.

160. The anti-TGFβ2 antibody of claim 153, wherein the anti-TGFβ2 antibody comprises a light chain variable region (VL) amino acid sequence having at least 95%, at least 96%, at least 97%, at least 98%, at least 99%, or 100% sequence identity to the amino acid sequence selected from the group consisting of SEQ ID NOs: 24, 131, 133-137, 143, and 144.

161. The anti-TGFβ2 antibody of claim 160, wherein the anti-TGFβ2 antibody comprises a light chain variable region (VL) amino acid sequence selected from the group consisting of SEQ ID NOs: 24, 131, 133-137, 143, and 144.

162. The anti-TGFβ2 antibody of claim 153, wherein the anti-TGFβ2 antibody comprises: (a) a heavy chain variable region (VH) comprising the amino acid sequence of SEQ ID NO: 132; and (b) a light chain variable region (VL) comprising the amino acid sequence of SEQ ID NO: 143.

163. The anti-TGFβ2 antibody of claim 153, wherein the anti-TGFβ2 antibody comprises a complete light (L) chain amino acid sequence having at least 95%, at least 96%, at least 97%, at least 98%, at least 99%, or 100% sequence identity to an amino acid sequence selected from the group consisting of SEQ ID NOs: 30, 145, 147-151, 157, and 158.

164. The anti-TGFβ2 antibody of claim 163, wherein the anti-TGFβ2 antibody comprises a complete light (L) chain amino acid sequence selected from the group consisting of SEQ ID NOs: 30, 145, 147-151, 157, and 158.

165. The anti-TGFβ2 antibody of claim 153, wherein the anti-TGFβ2 antibody comprises a heavy chain variable region (VH) and a light chain variable region (VL), wherein the heavy chain variable region and the light chain variable region comprise amino acid sequences (respectively) selected from the group consisting of SEQ ID NOs: 25/24 (rabbit 6F12), SEQ ID NOs: 132/131 (v1), SEQ ID NOs: 132/133 (v1.1), SEQ ID NOs: 132/134 (v1.2), SEQ ID NOs: 132/135 (v1.3), SEQ ID NOs: 132/136 (v1.4), SEQ ID NOs: 132/137 (v1.5), SEQ ID NOs: 138/131 (v1.6), SEQ ID NOs: 139/131 (v1.7), SEQ ID NOs: 140/131 (v1.8), SEQ ID NOs: 141/131 (v1.9), SEQ ID NOs: 142/131 (v2), SEQ ID NOs: 132/143 (v3), and SEQ ID NOs: 142/144 (v4).

166. The anti-TGFβ2 antibody of claim 153, wherein the anti-TGFβ2 antibody comprises a complete heavy (H) chain and complete light (L), chain wherein the complete heavy chain and the complete light chain comprise amino acid sequences (respectively) selected from the group consisting of SEQ ID NOs: 31/30 (rabbit 6F12), SEQ ID NOs: 146/145 (v1), SEQ ID NOs: 146/147 (v1.1), SEQ ID NOs: 146/148 (v1.2), SEQ ID NOs: 146/149 (v1.3), SEQ ID NOs: 146/150 (v1.4), SEQ ID NOs: 146/151 (v1.5), SEQ ID NOs: 152/145 (v1.6), SEQ ID NOs: 153/145 (v1.7), SEQ ID NOs: 154/145 (v1.8), SEQ ID NOs: 155/145 (v1.9), SEQ ID NOs: 156/145 (v2), SEQ ID NOs: 146/157 (v3), and SEQ ID NOs: 156/158 (v4).

167. The anti-TGFβ2 antibody of claim 153, wherein the anti-TGFβ2 antibody comprises a light chain variable region (VL) comprising the amino acid sequence of SEQ ID NO: 24 comprising one or more framework mutations selected from the group consisting of 43S or 43A, 66G, 69T, 71F, and 87Y.

168. The anti-TGFβ2 antibody of claim 167, wherein the light chain variable region (VL) comprises a set of framework mutations selected from the group consisting of:
(i) 43S in FR2 and 66E, 69P, 71Y and 87F in FR3 (h6F12.v1 and h6F12.v2);
(ii) 43S in FR2 and 58V, 66E, 69P, 71Y and 87F in FR3 (h6F12.v3 and h6F12.v4);
(iii) 43A in FR2 (h6F12.v1.1);
(iv) 66G in FR3 (h6F12.v1.2);
(v) 69T in FR3 (h6F12.v1.3);
(vi) 71F in FR3 (h6F12.v1.4); and
(vii) 87Y in FR3 (h6F12.v1.5).

169. The anti-TGFβ2 antibody of claim 153, wherein the anti-TGFβ2 antibody comprises a heavy chain variable region (VH) comprising the amino acid sequence of SEQ ID NO: 25 comprising one or more framework mutations selected from the group consisting of 37V or 37I, 48M or 48L, 49G or 49A, 67L, 71K and 78V, and 105P or 105R.

170. The anti-TGFβ2 antibody of claim 169, wherein the heavy chain variable region (VH) comprises a set of framework mutations selected from the group consisting of:
(i) 37V, 48M and 49G in FR2 and 105P in FR4 (h6F12.v1 and h6F12.v3);
(ii) 37V and 48M in FR2, 67L, 71K and 78V in FR3 and 105P in FR4 (h6F12.v2 and h6F12.v4);
(iii) 37I in FR2 (h6F12.v1.6);
(iv) 48L in FR2 (h6F12.v1.7);
(v) 49A in FR2 (h6F12.v1.8);
(vi) 105R in FR4 (h6F12.v1.9);
(vii) 37V, 48M and 49G in FR2 and 105P in FR4 (h6F12.v1 and h6F12.v3);
(viii) 37V and 48M in FR2, 67L, 71K and 78V in FR3 and 105P in FR4 (6F12.v2 and h6F12.v4);
(ix) 37I in FR2 (h6F12.v1.6);
(x) 48L in FR2 (h6F12.v1.7);
(xi) 49A in FR2 (h6F12.v1.8); and
(xii) 105R in FR4 (h6F12.v1.9).

171. The isolated anti-TGFβ2 antibody of claim 153, wherein the antibody comprises a heavy (H) chain comprising the amino acid sequence of SEQ ID NO: 146 and a light (L) chain comprising the amino acid sequence of SEQ ID NO: 157.

172. The anti-TGFβ2 antibody of claim 153, wherein the anti-TGFβ2 antibody specifically binds to human TGFβ2.

173. The anti-TGFβ2 antibody of claim 153, wherein the anti-TGFβ2 antibody is a monoclonal antibody.

174. The anti-TGFβ2 antibody of claim 153, wherein the anti-TGFβ2 antibody is a human antibody, a humanized antibody, or a chimeric antibody.

175. The anti-TGFβ2 antibody of claim 153, wherein the anti-TGFβ2 antibody is an antibody fragment.

176. The anti-TGFβ2 antibody of claim 153, wherein the anti-TGFβ2 antibody comprises a human Fc region that is an IgG1 or an IgG4 isotype.

177. The anti-TGFβ2 antibody of claim 176, wherein the anti-TGFβ2 antibody comprises a human Fc region that is an IgG1 isotype.

178. The anti-TGFβ2 antibody of claim 176, wherein the human Fc region comprises a modification to remove the glycosylation site at amino acid residue position N297 (EU numbering as in Kabat).

179. The anti-TGFβ2 antibody of claim 178, wherein the modification is a mutation selected from N297G or N297A.

180. The anti-TGFβ2 antibody of claim 179, wherein the modification is the mutation N297G.

181. The anti-TGFβ2 antibody of claim 176, wherein the human Fc region is modified to remove effector function.

182. The anti-TGFβ2 antibody of claim 153, wherein the anti-TGFβ2 antibody has a Cₘₐₓ of about 230-260 g/mL or a half life (t½) of about 15 to 16 days.

183. An immunoconjugate comprising the anti-TGFβ2 antibody of claim 153 and a cytotoxic agent.

184. A pharmaceutical formulation comprising the anti-TGFβ2 antibody of claim 153, and a pharmaceutically acceptable carrier.

185. A pharmaceutical formulation comprising the anti-TGFβ2 antibody of claim 153, wherein the pharmaceutical formulation further comprises an additional therapeutic agent.

186. The pharmaceutical formulation of claim 185, wherein the additional therapeutic agent is selected from the group consisting of pirfenidone, nintedanib, mycophenylate mofetil, an IL-6 inhibitor, an anti-CTFG antibody, an autotaxin inhibitor, a JAK inhibitor, an IL-11 inhibitor, and PTX2.

187. The pharmaceutical formulation of claim 186, wherein the IL-6 inhibitor comprises tocilizumab and the anti-CTFG antibody comprises FG-3019.

188. A kit comprising the anti-TGFβ2 antibody of claim 153.


SEQUENCE LISTING (188 sequences)

SEQ ID NO: 1 - protein, 390 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 2 - protein, 414 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 3 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 4 - protein, 5 aa, synthetic construct
  REGION 1..5: note = Description of Artificial Sequence: Synthetic peptide
SYGMS

SEQ ID NO: 5 - protein, 19 aa, synthetic construct
  REGION 1..19: note = Description of Artificial Sequence: Synthetic peptide
DIVSKTYNYTTYYSDSVKD

SEQ ID NO: 6 - protein, 8 aa, synthetic construct
  REGION 1..8: note = Description of Artificial Sequence: Synthetic peptide
APGGSFDY

SEQ ID NO: 7 - protein, 15 aa, synthetic construct
  REGION 1..15: note = Description of Artificial Sequence: Synthetic peptide
RASQSVSISRFNLMH

SEQ ID NO: 8 - protein, 7 aa, synthetic construct
  REGION 1..7: note = Description of Artificial Sequence: Synthetic peptide
RASNLAS

SEQ ID NO: 9 - protein, 9 aa, synthetic construct
  REGION 1..9: note = Description of Artificial Sequence: Synthetic peptide
QHSRESPWT

SEQ ID NO: 10 - protein, 5 aa, synthetic construct
  REGION 1..5: note = Description of Artificial Sequence: Synthetic peptide
SYTVN

SEQ ID NO: 11 - protein, 16 aa, synthetic construct
  REGION 1..16: note = Description of Artificial Sequence: Synthetic peptide
YISYGGSAYYASWANG

SEQ ID NO: 12 - protein, 17 aa, synthetic construct
  REGION 1..17: note = Description of Artificial Sequence: Synthetic peptide
HMQVGGAPTGSMAAFDP

SEQ ID NO: 13 - protein, 13 aa, synthetic construct
  REGION 1..13: note = Description of Artificial Sequence: Synthetic peptide
QSSQSVYNNNYLS

SEQ ID NO: 14 - protein, 7 aa, synthetic construct
  REGION 1..7: note = Description of Artificial Sequence: Synthetic peptide
GASTLTS

SEQ ID NO: 15 - protein, 12 aa, synthetic construct
  REGION 1..12: note = Description of Artificial Sequence: Synthetic peptide
AGGYSGSSDKYA

SEQ ID NO: 16 - protein, 5 aa, synthetic construct
  REGION 1..5: note = Description of Artificial Sequence: Synthetic peptide
TYNVH

SEQ ID NO: 17 - protein, 16 aa, synthetic construct
  REGION 1..16: note = Description of Artificial Sequence: Synthetic peptide
LIWNTGGTRYNSALKS

SEQ ID NO: 18 - protein, 11 aa, synthetic construct
  REGION 1..11: note = Description of Artificial Sequence: Synthetic peptide
DPVPNKWHFDF

SEQ ID NO: 19 - protein, 11 aa, synthetic construct
  REGION 1..11: note = Description of Artificial Sequence: Synthetic peptide
LASEDIYSNLA

SEQ ID NO: 20 - protein, 7 aa, synthetic construct
  REGION 1..7: note = Description of Artificial Sequence: Synthetic peptide
DARSLQD

SEQ ID NO: 21 - protein, 9 aa, synthetic construct
  REGION 1..9: note = Description of Artificial Sequence: Synthetic peptide
QQHHAYPFT

SEQ ID NO: 22 - protein, 110 aa, synthetic construct
  REGION 1..110: note = Description of Artificial Sequence: Synthetic polypeptide
DIVLTQSPALAVSLGQRATISCRASQSVSISRFNLMHWYQHKPGQQPKLLIYRASNLASGIPARFSGSGSGTDFTLTINPVQADDLATYYCQHSRESPWTFGGGTKLEIK

SEQ ID NO: 23 - protein, 119 aa, synthetic construct
  REGION 1..119: note = Description of Artificial Sequence: Synthetic polypeptide
EVQLVESGGGLVQPKGSLKLSCAASGFDFNSYGMSWVRQAPGKGLDLVADIVSKTYNYTTYYSDSVKDRFTISRDDSQSMVYLQMDNLKTEDTALYYCTVAPGGSFDYWGQGVMVTVSS

SEQ ID NO: 24 - protein, 107 aa, synthetic construct
  REGION 1..107: note = Description of Artificial Sequence: Synthetic polypeptide
DIQMTQSPASLSASLGETVTIECLASEDIYSNLAWYQQKPGKSPQLLIYDARSLQDGVPSRFSGSESGPQYSLEINSLQSEDAVTYFCQQHHAYPFTFGSGTKLEIK

SEQ ID NO: 25 - protein, 119 aa, synthetic construct
  REGION 1..119: note = Description of Artificial Sequence: Synthetic polypeptide
QVQLKESGPGLVQPSQTLSLTCTVSGFSLTTYNVHWVRQPPGKGLEWMGLIWNTGGTRYNSALKSRLSISKDTSKSQVFLRMNSLQTEDTATYYCARDPVPNKWHFDFWGPGTMVTVSS

SEQ ID NO: 26 - protein, 112 aa, synthetic construct
  REGION 1..112: note = Description of Artificial Sequence: Synthetic polypeptide
AAVLTQTPSPVSAAVGGTVSISCQSSQSVYNNNYLSWFQQKPGQPPKLLIYGASTLTSGVPSRFKGSGSGTQFTLTISDVQCDDAATYYCAGGYSGSSDKYAFGGGTEVVVK

SEQ ID NO: 27 - protein, 122 aa, synthetic construct
  REGION 1..122: note = Description of Artificial Sequence: Synthetic polypeptide
QSLEESGGRLVTPGTPLTLTCTVSGFSLSSYTVNWVRQAPGKGLEWIGYISYGGSAYYASWANGRFTISKTSATVDLKITSPTTEDTATYFCARHMQVGGAPTGSMAAFDPWGPGTLVTVSS

SEQ ID NO: 28 - protein, 217 aa, synthetic construct
  REGION 1..217: note = Description of Artificial Sequence: Synthetic polypeptide
DIVLTQSPALAVSLGQRATISCRASQSVSISRFNLMHWYQHKPGQQPKLLIYRASNLASGIPARFSGSGSGTDFTLTINPVQADDLATYYCQHSRESPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 29 - protein, 449 aa, synthetic construct
  REGION 1..449: note = Description of Artificial Sequence: Synthetic polypeptide
EVQLVESGGGLVQPKGSLKLSCAASGFDFNSYGMSWVRQAPGKGLDLVADIVSKTYNYTTYYSDSVKDRFTISRDDSQSMVYLQMDNLKTEDTALYYCTVAPGGSFDYWGQGVMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 30 - protein, 214 aa, synthetic construct
  REGION 1..214: note = Description of Artificial Sequence: Synthetic polypeptide
DIQMTQSPASLSASLGETVTIECLASEDIYSNLAWYQQKPGKSPQLLIYDARSLQDGVPSRFSGSESGPQYSLEINSLQSEDAVTYFCQQHHAYPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 31 - protein, 449 aa, synthetic construct
  REGION 1..449: note = Description of Artificial Sequence: Synthetic polypeptide
QVQLKESGPGLVQPSQTLSLTCTVSGFSLTTYNVHWVRQPPGKGLEWMGLIWNTGGTRYNSALKSRLSISKDTSKSQVFLRMNSLQTEDTATYYCARDPVPNKWHFDFWGPGTMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 32 - protein, 216 aa, synthetic construct
  REGION 1..216: note = Description of Artificial Sequence: Synthetic polypeptide
AAVLTQTPSPVSAAVGGTVSISCQSSQSVYNNNYLSWFQQKPGQPPKLLIYGASTLTSGVPSRFKGSGSGTQFTLTISDVQCDDAATYYCAGGYSGSSDKYAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 33 - protein, 445 aa, synthetic construct
  REGION 1..445: note = Description of Artificial Sequence: Synthetic polypeptide
QSLEESGGRLVTPGTPLTLTCTVSGFSLSSYTVNWVRQAPGKGLEWIGYISYGGSAYYASWANGRFTISKTSATVDLKITSPTTEDTATYFCARHMQVGGAPTGSMAAFDPWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 34 - protein, 19 aa, synthetic construct
  REGION 1..19: note = Description of Artificial Sequence: Synthetic peptide
DIVSKTYSYTTYYSDSVKD

SEQ ID NO: 35 - protein, 19 aa, synthetic construct
  REGION 1..19: note = Description of Artificial Sequence: Synthetic peptide
DIVSKTYNYATYYSDSVKD

SEQ ID NO: 36 - protein, 111 aa, synthetic construct
  REGION 1..111: note = Description of Artificial Sequence: Synthetic polypeptide
DIQLTQSPSSLSASVGDRVTITCRASQSVSISRFNLMHWYQHKPGKQPKLLIYRASNLASGIPSRFSGSGSGTDFTLTISSLQPEDFATYYCQHSRESPWTFGGGTKVEIK

SEQ ID NO: 37 - protein, 119 aa, synthetic construct
  REGION 1..119: note = Description of Artificial Sequence: Synthetic polypeptide
EVQLVESGGGLVQPGPSLRLSCTASGFDFNSYGMSWVRQAPGKGLELVADIVSKTYNYTTYYSDSVKDRFTISRDDSKSIVYLQMNSLKTEDTAVYYCTVAPGGSFDYWGQGTLVTVSS

SEQ ID NO: 38 - protein, 111 aa, synthetic construct
  REGION 1..111: note = Description of Artificial Sequence: Synthetic polypeptide
DIQMTQSPSSLSASVGDRVTITCRASQSVSISRFNLMHWYQHKPGKQPKLLIYRASNLASGIPSRFSGSGSGTDFTLTISSLQPEDFATYYCQHSRESPWTFGGGTKVEIK

SEQ ID NO: 39 - protein, 111 aa, synthetic construct
  REGION 1..111: note = Description of Artificial Sequence: Synthetic polypeptide
DIQLTQSPSSLSASVGDRVTITCRASQSVSISRFNLMHWYQQKPGKQPKLLIYRASNLASGIPSRFSGSGSGTDFTLTISSLQPEDFATYYCQHSRESPWTFGGGTKVEIK

SEQ ID NO: 40 - protein, 111 aa, synthetic construct
  REGION 1..111: note = Description of Artificial Sequence: Synthetic polypeptide
DIQLTQSPSSLSASVGDRVTITCRASQSVSISRFNLMHWYQHKPGKAPKLLIYRASNLASGIPSRFSGSGSGTDFTLTISSLQPEDFATYYCQHSRESPWTFGGGTKVEIK

SEQ ID NO: 41 - protein, 111 aa, synthetic construct
  REGION 1..111: note = Description of Artificial Sequence: Synthetic polypeptide
DIQLTQSPSSLSASVGDRVTITCRASQSVSISRFNLMHWYQHKPGKQPKLLIYRASNLASGVPSRFSGSGSGTDFTLTISSLQPEDFATYYCQHSRESPWTFGGGTKVEIK

SEQ ID NO: 42 - protein, 119 aa, synthetic construct
  REGION 1..119: note = Description of Artificial Sequence: Synthetic polypeptide
EVQLVESGGGLVQPGPSLRLSCTASGFDFNSYGMSWVRQAPGKGLEWVADIVSKTYNYTTYYSDSVKDRFTISRDDSKSIVYLQMNSLKTEDTAVYYCTVAPGGSFDYWGQGTLVTVSS

SEQ ID NO: 43 - protein, 119 aa, synthetic construct
  REGION 1..119: note = Description of Artificial Sequence: Synthetic polypeptide
EVQLVESGGGLVQPGPSLRLSCTASGFDFNSYGMSWVRQAPGKGLELVGDIVSKTYNYTTYYSDSVKDRFTISRDDSKSIVYLQMNSLKTEDTAVYYCTVAPGGSFDYWGQGTLVTVSS

SEQ ID NO: 44 - protein, 119 aa, synthetic construct
  REGION 1..119: note = Description of Artificial Sequence: Synthetic polypeptide
EVQLVESGGGLVQPGPSLRLSCTASGFDFNSYGMSWVRQAPGKGLELVADIVSKTYNYTTYYSDSVKDRFTISRDDSKSIAYLQMNSLKTEDTAVYYCTVAPGGSFDYWGQGTLVTVSS

SEQ ID NO: 45 - protein, 119 aa, synthetic construct
  REGION 1..119: note = Description of Artificial Sequence: Synthetic polypeptide
EVQLLESGGGLVQPGGSLRLSCAASGFDFNSYGMSWVRQAPGKGLELVADIVSKTYNYTTYYSDSVKDRFTISRDDSKSTVYLQMNSLRAEDTAVYYCTVAPGGSFDYWGQGTLVTVSS

SEQ ID NO: 46 - protein, 119 aa, synthetic construct
  REGION 1..119: note = Description of Artificial Sequence: Synthetic polypeptide
EVQLLESGGGLVQPGGSLRLSCAASGFDFNSYGMSWVRQAPGKGLEWVADIVSKTYNYTTYYSDSVKDRFTISRDDSKSTVYLQMNSLRAEDTAVYYCTVAPGGSFDYWGQGTLVTVSS

SEQ ID NO: 47 - protein, 119 aa, synthetic construct
  REGION 1..119: note = Description of Artificial Sequence: Synthetic polypeptide
EVQLLESGGGLVQPGGSLRLSCAASGFDFNSYGMSWVRQAPGKGLELVSDIVSKTYNYTTYYSDSVKDRFTISRDDSKSTVYLQMNSLRAEDTAVYYCTVAPGGSFDYWGQGTLVTVSS

SEQ ID NO: 48 - protein, 119 aa, synthetic construct
  REGION 1..119: note = Description of Artificial Sequence: Synthetic polypeptide
EVQLLESGGGLVQPGGSLRLSCAASGFDFNSYGMSWVRQAPGKGLELVADIVSKTYNYTTYYSDSVKDRFTISRDNSKSTVYLQMNSLRAEDTAVYYCTVAPGGSFDYWGQGTLVTVSS

SEQ ID NO: 49 - protein, 119 aa, synthetic construct
  REGION 1..119: note = Description of Artificial Sequence: Synthetic polypeptide
EVQLLESGGGLVQPGGSLRLSCAASGFDFNSYGMSWVRQAPGKGLELVADIVSKTYNYTTYYSDSVKDRFTISRDDSKNTVYLQMNSLRAEDTAVYYCTVAPGGSFDYWGQGTLVTVSS

SEQ ID NO: 50 - protein, 119 aa, synthetic construct
  REGION 1..119: note = Description of Artificial Sequence: Synthetic polypeptide
EVQLLESGGGLVQPGGSLRLSCAASGFDFNSYGMSWVRQAPGKGLELVADIVSKTYNYTTYYSDSVKDRFTISRDDSKSTLYLQMNSLRAEDTAVYYCTVAPGGSFDYWGQGTLVTVSS

SEQ ID NO: 51 - protein, 119 aa, synthetic construct
  REGION 1..119: note = Description of Artificial Sequence: Synthetic polypeptide
EVQLLESGGGLVQPGGSLRLSCAASGFDFNSYGMSWVRQAPGKGLELVADIVSKTYSYTTYYSDSVKDRFTISRDDSKSTVYLQMNSLRAEDTAVYYCTVAPGGSFDYWGQGTLVTVSS

SEQ ID NO: 52 - protein, 119 aa, synthetic construct
  REGION 1..119: note = Description of Artificial Sequence: Synthetic polypeptide
EVQLLESGGGLVQPGGSLRLSCAASGFDFNSYGMSWVRQAPGKGLELVADIVSKTYQYTTYYSDSVKDRFTISRDDSKSTVYLQMNSLRAEDTAVYYCTVAPGGSFDYWGQGTLVTVSS

SEQ ID NO: 53 - protein, 119 aa, synthetic construct
  REGION 1..119: note = Description of Artificial Sequence: Synthetic polypeptide
EVQLLESGGGLVQPGGSLRLSCAASGFDFNSYGMSWVRQAPGKGLELVADIVSKTYNYATYYSDSVKDRFTISRDDSKSTVYLQMNSLRAEDTAVYYCTVAPGGSFDYWGQGTLVTVSS

SEQ ID NO: 54 - protein, 111 aa, synthetic construct
  REGION 1..111: note = Description of Artificial Sequence: Synthetic polypeptide
DIQLTQSPSSLSASVGDRVTITCRASQSVSISRFNLMHWYQQKPGKAPKLLIYRASNLASGVPSRFSGSGSGTDFTLTISSLQPEDFATYYCQHSRESPWTFGGGTKVEIK

SEQ ID NO: 55 - protein, 119 aa, synthetic construct
  REGION 1..119: note = Description of Artificial Sequence: Synthetic polypeptide
EVQLLESGGGLVQPGGSLRLSCAASGFDFNSYGMSWVRQAPGKGLELVSDIVSKTYSYTTYYSDSVKDRFTISRDDSKNTLYLQMNSLRAEDTAVYYCTVAPGGSFDYWGQGTLVTVSS

SEQ ID NO: 56 - protein, 111 aa, synthetic construct; identical to SEQ ID NO: 54
  REGION 1..111: note = Description of Artificial Sequence: Synthetic polypeptide

SEQ ID NO: 57 - protein, 119 aa, synthetic construct
  REGION 1..119: note = Description of Artificial Sequence: Synthetic polypeptide
EVQLLESGGGLVQPGGSLRLSCAASGFDFNSYGMSWVRQAPGKGLELVSDIVSKTYNYATYYSDSVKDRFTISRDDSKNTLYLQMNSLRAEDTAVYYCTVAPGGSFDYWGQGTLVTVSS

SEQ ID NO: 58 - protein, 218 aa, synthetic construct
  REGION 1..218: note = Description of Artificial Sequence: Synthetic polypeptide
DIQLTQSPSSLSASVGDRVTITCRASQSVSISRFNLMHWYQHKPGKQPKLLIYRASNLASGIPSRFSGSGSGTDFTLTISSLQPEDFATYYCQHSRESPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 449 aa, synthetic construct
  REGION 1..449: note = Description of Artificial Sequence: Synthetic polypeptide
EVQLVESGGGLVQPGPSLRLSCTASGFDFNSYGMSWVRQAPGKGLELVADIVSKTYNYTTYYSDSVKDRFTISRDDSKSIVYLQMNSLKTEDTAVYYCTVAPGGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 218 aa, synthetic construct
  REGION 1..218: note = Description of Artificial Sequence: Synthetic polypeptide
DIQMTQSPSSLSASVGDRVTITCRASQSVSISRFNLMHWYQHKPGKQPKLLIYRASNLASGIPSRFSGSGSGTDFTLTISSLQPEDFATYYCQHSRESPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 218 aa, synthetic construct
  REGION 1..218: note = Description of Artificial Sequence: Synthetic polypeptide
DIQLTQSPSSLSASVGDRVTITCRASQSVSISRFNLMHWYQQKPGKQPKLLIYRASNLASGIPSRFSGSGSGTDFTLTISSLQPEDFATYYCQHSRESPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 218 aa, synthetic construct
  REGION 1..218: note = Description of Artificial Sequence: Synthetic polypeptide
DIQLTQSPSSLSASVGDRVTITCRASQSVSISRFNLMHWYQHKPGKAPKLLIYRASNLASGIPSRFSGSGSGTDFTLTISSLQPEDFATYYCQHSRESPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 218 aa, synthetic construct
  REGION 1..218: note = Description of Artificial Sequence: Synthetic polypeptide
DIQLTQSPSSLSASVGDRVTITCRASQSVSISRFNLMHWYQHKPGKQPKLLIYRASNLASGVPSRFSGSGSGTDFTLTISSLQPEDFATYYCQHSRESPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 449 aa, synthetic construct
  REGION 1..449: note = Description of Artificial Sequence: Synthetic polypeptide
EVQLVESGGGLVQPGPSLRLSCTASGFDFNSYGMSWVRQAPGKGLEWVADIVSKTYNYTTYYSDSVKDRFTISRDDSKSIVYLQMNSLKTEDTAVYYCTVAPGGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 449 aa, synthetic construct
  REGION 1..449: note = Description of Artificial Sequence: Synthetic polypeptide
EVQLVESGGGLVQPGPSLRLSCTASGFDFNSYGMSWVRQAPGKGLELVGDIVSKTYNYTTYYSDSVKDRFTISRDDSKSIVYLQMNSLKTEDTAVYYCTVAPGGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 449 aa, synthetic construct
  REGION 1..449: note = Description of Artificial Sequence: Synthetic polypeptide
EVQLVESGGGLVQPGPSLRLSCTASGFDFNSYGMSWVRQAPGKGLELVADIVSKTYNYTTYYSDSVKDRFTISRDDSKSIAYLQMNSLKTEDTAVYYCTVAPGGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 449 aa, synthetic construct
  REGION 1..449: note = Description of Artificial Sequence: Synthetic polypeptide
EVQLLESGGGLVQPGGSLRLSCAASGFDFNSYGMSWVRQAPGKGLELVADIVSKTYNYTTYYSDSVKDRFTISRDDSKSTVYLQMNSLRAEDTAVYYCTVAPGGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 449 aa, synthetic construct
  REGION 1..449: note = Description of Artificial Sequence: Synthetic polypeptide
EVQLLESGGGLVQPGGSLRLSCAASGFDFNSYGMSWVRQAPGKGLEWVADIVSKTYNYTTYYSDSVKDRFTISRDDSKSTVYLQMNSLRAEDTAVYYCTVAPGGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 449 aa, synthetic construct
  REGION 1..449: note = Description of Artificial Sequence: Synthetic polypeptide
EVQLLESGGGLVQPGGSLRLSCAASGFDFNSYGMSWVRQAPGKGLELVSDIVSKTYNYTTYYSDSVKDRFTISRDDSKSTVYLQMNSLRAEDTAVYYCTVAPGGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 449 aa, synthetic construct
  REGION 1..449: note = Description of Artificial Sequence: Synthetic polypeptide
EVQLLESGGGLVQPGGSLRLSCAASGFDFNSYGMSWVRQAPGKGLELVADIVSKTYNYTTYYSDSVKDRFTISRDNSKSTVYLQMNSLRAEDTAVYYCTVAPGGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 449 aa, synthetic construct
  REGION 1..449: note = Description of Artificial Sequence: Synthetic polypeptide
EVQLLESGGGLVQPGGSLRLSCAASGFDFNSYGMSWVRQAPGKGLELVADIVSKTYNYTTYYSDSVKDRFTISRDDSKNTVYLQMNSLRAEDTAVYYCTVAPGGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 449 aa, synthetic construct
  REGION 1..449: note = Description of Artificial Sequence: Synthetic polypeptide
EVQLLESGGGLVQPGGSLRLSCAASGFDFNSYGMSWVRQAPGKGLELVADIVSKTYNYTTYYSDSVKDRFTISRDDSKSTLYLQMNSLRAEDTAVYYCTVAPGGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 449 aa, synthetic construct
  REGION 1..449: note = Description of Artificial Sequence: Synthetic polypeptide
EVQLLESGGGLVQPGGSLRLSCAASGFDFNSYGMSWVRQAPGKGLELVADIVSKTYSYTTYYSDSVKDRFTISRDDSKSTVYLQMNSLRAEDTAVYYCTVAPGGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 449 aa, synthetic construct
  REGION 1..449: note = Description of Artificial Sequence: Synthetic polypeptide
EVQLLESGGGLVQPGGSLRLSCAASGFDFNSYGMSWVRQAPGKGLELVADIVSKTYQYTTYYSDSVKDRFTISRDDSKSTVYLQMNSLRAEDTAVYYCTVAPGGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 449 aa, synthetic construct
  REGION 1..449: note = Description of Artificial Sequence: Synthetic polypeptide
EVQLLESGGGLVQPGGSLRLSCAASGFDFNSYGMSWVRQAPGKGLELVADIVSKTYNYATYYSDSVKDRFTISRDDSKSTVYLQMNSLRAEDTAVYYCTVAPGGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 218 aa, synthetic construct
  REGION 1..218: note = Description of Artificial Sequence: Synthetic polypeptide
DIQLTQSPSSLSASVGDRVTITCRASQSVSISRFNLMHWYQQKPGKAPKLLIYRASNLASGVPSRFSGSGSGTDFTLTISSLQPEDFATYYCQHSRESPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 449 aa, synthetic construct
  REGION 1..449: note = Description of Artificial Sequence: Synthetic polypeptide
EVQLLESGGGLVQPGGSLRLSCAASGFDFNSYGMSWVRQAPGKGLELVSDIVSKTYSYTTYYSDSVKDRFTISRDDSKNTLYLQMNSLRAEDTAVYYCTVAPGGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 218 aa, synthetic construct; identical to SEQ ID NO: 76
  REGION 1..218: note = Description of Artificial Sequence: Synthetic polypeptide

SEQ ID NO: 79 - protein, 449 aa, synthetic construct
  REGION 1..449: note = Description of Artificial Sequence: Synthetic polypeptide
EVQLLESGGGLVQPGGSLRLSCAASGFDFNSYGMSWVRQAPGKGLELVSDIVSKTYNYATYYSDSVKDRFTISRDDSKNTLYLQMNSLRAEDTAVYYCTVAPGGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 112 aa, synthetic construct
  REGION 1..112: note = Description of Artificial Sequence: Synthetic polypeptide
DAVLTQSPDSLAVSLGERATINCQSSQSVYNNNYLSWFQQKPGQPPKLLIYGASTLTSGVPDRFSGSGSGTDFTLTISSLQAEDVAVYYCAGGYSGSSDKYAFGGGTKVEIK

SEQ ID NO: 81 - protein, 125 aa, synthetic construct
  REGION 1..125: note = Description of Artificial Sequence: Synthetic polypeptide
EQQLVESGGGLVQPGGSLRLSCAVSGFSLSSYTVNWVRQAPGKGLEWIGYISYGGSAYYASWANGRFTISKDSAKNSVYLQMNSLRAEDTAVYFCARHMQVGGAPTGSMAAFDPWGPGTLVTVSS

SEQ ID NO: 82 - protein, 112 aa, synthetic construct
  REGION 1..112: note = Description of Artificial Sequence: Synthetic polypeptide
DAQLTQSPSSLSASVGDRVTITCQSSQSVYNNNYLSWFQQKPGKPPKLLIYGASTLTSGVPSRFSGSGSGTDFTLTISSLQPEDFATYYCAGGYSGSSDKYAFGGGTKVEIK

SEQ ID NO: 83 - protein, 125 aa, synthetic construct
  REGION 1..125: note = Description of Artificial Sequence: Synthetic polypeptide
EQQLQESGPGLVKPSETLSLTCTVTGFSLSSYTVNWVRQPAGKGLEWIGYISYGGSAYYASWANGRFTISKDSSKNQVSLKLSSVTAADTAVYFCARHMQVGGAPTGSMAAFDPWGPGTLVTVSS

SEQ ID NO: 84 - protein, 112 aa, synthetic construct
  REGION 1..112: note = Description of Artificial Sequence: Synthetic polypeptide
EAVLTQSPGTLSLSPGERATLSCQSSQSVYNNNYLSWFQQKPGQPPRLLIYGASTLTSGVPDRFSGSGSGTDFTLTISRLEPEDFAVYYCAGGYSGSSDKYAFGGGTKVEIK

SEQ ID NO: 85 - protein, 112 aa, synthetic construct
  REGION 1..112: note = Description of Artificial Sequence: Synthetic polypeptide
DAVLTQTPLSSPVTLGQPASISCQSSQSVYNNNYLSWFQQRPGQPPRLLIYGASTLTSGVPDRFSGSGAGTDFTLKISRVEAEDVGVYYCAGGYSGSSDKYAFGGGTKVEIK

SEQ ID NO: 86 - protein, 124 aa, synthetic construct
  REGION 1..124: note = Description of Artificial Sequence: Synthetic polypeptide
QQLQESGPGLVKPSETLSLTCTVTGFSLSSYTVNWVRQPAGKGLEWIGYISYGGSAYYASWANGRFTISKDSSKNQVSLKLSSVTAADTAVYFCARHMQVGGAPTGSMAAFDPWGPGTLVTVSS

SEQ ID NO: 87 - protein, 123 aa, synthetic construct
  REGION 1..123: note = Description of Artificial Sequence: Synthetic polypeptide
EQQLQESGPGLVKPSETLSLTCTVTGFSLSSYTVNWVRQPAGKGLEWIGYISYGGSAYYASWANGRFTISKDSSQVSLKLSSVTAADTAVYFCARHMQVGGAPTGSMAAFDPWGPGTLVTVSS

SEQ ID NO: 88 - protein, 122 aa, synthetic construct
  REGION 1..122: note = Description of Artificial Sequence: Synthetic polypeptide
QQLQESGPGLVKPSETLSLTCTVTGFSLSSYTVNWVRQPAGKGLEWIGYISYGGSAYYASWANGRFTISKDSSQVSLKLSSVTAADTAVYFCARHMQVGGAPTGSMAAFDPWGPGTLVTVSS

SEQ ID NO: 89 - protein, 112 aa, synthetic construct
  REGION 1..112: note = Description of Artificial Sequence: Synthetic polypeptide
EIVLTQSPGTLSLSPGERATLSCQSSQSVYNNNYLSWFQQKPGQPPRLLIYGASTLTSGVPDRFSGSGSGTDFTLTISRLEPEDFAVYYCAGGYSGSSDKYAFGGGTKVEIK

SEQ ID NO: 90 - protein, 112 aa, synthetic construct
  REGION 1..112: note = Description of Artificial Sequence: Synthetic polypeptide
EAVLTQSPGTLSLSPGERATLSCQSSQSVYNNNYLSWYQQKPGQPPRLLIYGASTLTSGVPDRFSGSGSGTDFTLTISRLEPEDFAVYYCAGGYSGSSDKYAFGGGTKVEIK

SEQ ID NO: 91 - protein, 112 aa, synthetic construct
  REGION 1..112: note = Description of Artificial Sequence: Synthetic polypeptide
EAVLTQSPGTLSLSPGERATLSCQSSQSVYNNNYLSWFQQKPGQAPRLLIYGASTLTSGVPDRFSGSGSGTDFTLTISRLEPEDFAVYYCAGGYSGSSDKYAFGGGTKVEIK

SEQ ID NO: 92 - protein, 112 aa, synthetic construct
  REGION 1..112: note = Description of Artificial Sequence: Synthetic polypeptide
EAVLTQSPGTLSLSPGERATLSCQSSQSVYNNNYLSWFQQKPGQPPRLLIYGASTLTSGIPDRFSGSGSGTDFTLTISRLEPEDFAVYYCAGGYSGSSDKYAFGGGTKVEIK

SEQ ID NO: 93 - protein, 125 aa, synthetic construct
  REGION 1..125: note = Description of Artificial Sequence: Synthetic polypeptide
EVQLQESGPGLVKPSETLSLTCTVTGFSLSSYTVNWVRQPAGKGLEWIGYISYGGSAYYASWANGRFTISKDSSKNQVSLKLSSVTAADTAVYFCARHMQVGGAPTGSMAAFDPWGPGTLVTVSS

SEQ ID NO: 94 - protein, 125 aa, synthetic construct
  REGION 1..125: note = Description of Artificial Sequence: Synthetic polypeptide
EQQLQESGPGLVKPSETLSLTCTVTGFSLSSYTVNWIRQPAGKGLEWIGYISYGGSAYYASWANGRFTISKDSSKNQVSLKLSSVTAADTAVYFCARHMQVGGAPTGSMAAFDPWGPGTLVTVSS

SEQ ID NO: 95 - protein, 125 aa, synthetic construct
  REGION 1..125: note = Description of Artificial Sequence: Synthetic polypeptide
EQQLQESGPGLVKPSETLSLTCTVTGFSLSSYTVNWVRQPAGKGLEWIGYISYGGSAYYASWANGRVTISKDSSKNQVSLKLSSVTAADTAVYFCARHMQVGGAPTGSMAAFDPWGPGTLVTVSS

SEQ ID NO: 96 - protein, 125 aa, synthetic construct
  REGION 1..125: note = Description of Artificial Sequence: Synthetic polypeptide
EQQLQESGPGLVKPSETLSLTCTVTGFSLSSYTVNWVRQPAGKGLEWIGYISYGGSAYYASWANGRFTISVDSSKNQVSLKLSSVTAADTAVYFCARHMQVGGAPTGSMAAFDPWGPGTLVTVSS

SEQ ID NO: 97 - protein, 125 aa, synthetic construct
  REGION 1..125: note = Description of Artificial Sequence: Synthetic polypeptide
EQQLQESGPGLVKPSETLSLTCTVTGFSLSSYTVNWVRQPAGKGLEWIGYISYGGSAYYASWANGRFTISKDTSKNQVSLKLSSVTAADTAVYFCARHMQVGGAPTGSMAAFDPWGPGTLVTVSS

SEQ ID NO: 98 - protein, 125 aa, synthetic construct
  REGION 1..125: note = Description of Artificial Sequence: Synthetic polypeptide
EQQLQESGPGLVKPSETLSLTCTVTGFSLSSYTVNWVRQPAGKGLEWIGYISYGGSAYYASWANGRFTISKDSSKNQFSLKLSSVTAADTAVYFCARHMQVGGAPTGSMAAFDPWGPGTLVTVSS

SEQ ID NO: 99 - protein, 125 aa, synthetic construct
  REGION 1..125: note = Description of Artificial Sequence: Synthetic polypeptide
EQQLQESGPGLVKPSETLSLTCTVTGFSLSSYTVNWVRQPAGKGLEWIGYISYGGSAYYASWANGRFTISKDSSKNQVSLKLSSVTAADTAVYYCARHMQVGGAPTGSMAAFDPWGPGTLVTVSS

SEQ ID NO: 100 - protein, 125 aa, synthetic construct
  REGION 1..125: note = Description of Artificial Sequence: Synthetic polypeptide
EQQLQESGPGLVKPSETLSLTCTVTGFSLSSYTVNWVRQPAGKGLEWIGYISYGGSAYYASWANGRFTISKDSSKNQVSLKLSSVTAADTAVYFCARHMQVGGAPTGSMAAFDPWGQGTLVTVSS

SEQ ID NO: 101 - protein, 112 aa, synthetic construct
  REGION 1..112: note = Description of Artificial Sequence: Synthetic polypeptide
EIVLTQSPGTLSLSPGERATLSCQSSQSVYNNNYLSWFQQKPGQAPRLLIYGASTLTSGIPDRFSGSGSGTDFTLTISRLEPEDFAVYYCAGGYSGSSDKYAFGGGTKVEIK

SEQ ID NO: 102 - protein, 125 aa, synthetic construct
  REGION 1..125: note = Description of Artificial Sequence: Synthetic polypeptide
EVQLQESGPGLVKPSETLSLTCTVTGFSLSSYTVNWIRQPAGKGLEWIGYISYGGSAYYASWANGRVTISKDTSKNQFSLKLSSVTAADTAVYFCARHMQVGGAPTGSMAAFDPWGQGTLVTVSS

SEQ ID NO: 103 - protein, 125 aa, synthetic construct
  REGION 1..125: note = Description of Artificial Sequence: Synthetic polypeptide
EVQLQESGPGLVKPSETLSLTCTVTGFSLSSYTVNWIRQPAGKGLEWIGYISYGGSAYYASWANGRVTISKDTSKNQVSLKLSSVTAADTAVYYCARHMQVGGAPTGSMAAFDPWGQGTLVTVSS

SEQ ID NO: 104 - protein, 125 aa, synthetic construct
  REGION 1..125: note = Description of Artificial Sequence: Synthetic polypeptide
EVQLQESGPGLVKPSETLSLTCTVTGFSLSSYTVNWIRQPAGKGLEWIGYISYGGSAYYASWANGRVTISKDTSKNQVSLKLSSVTAADTAVYFCARHMQVGGAPTGSMAAFDPWGQGTLVTVSS

SEQ ID NO: 105 - protein, 123 aa, synthetic construct
  REGION 1..123: note = Description of Artificial Sequence: Synthetic polypeptide
EVQLQESGPGLVKPSETLSLTCTVTGFSLSSYTVNWIRQPAGKGLEWIGYISYGGSAYYASWANGRVTISKDTSQVSLKLSSVTAADTAVYFCARHMQVGGAPTGSMAAFDPWGQGTLVTVSS

SEQ ID NO: 106 - protein, 219 aa, synthetic construct
  REGION 1..219: note = Description of Artificial Sequence: Synthetic polypeptide
DAVLTQSPDSLAVSLGERATINCQSSQSVYNNNYLSWFQQKPGQPPKLLIYGASTLTSGVPDRFSGSGSGTDFTLTISSLQAEDVAVYYCAGGYSGSSDKYAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 455 aa, synthetic construct
  REGION 1..455: note = Description of Artificial Sequence: Synthetic polypeptide
EQQLVESGGGLVQPGGSLRLSCAVSGFSLSSYTVNWVRQAPGKGLEWIGYISYGGSAYYASWANGRFTISKDSAKNSVYLQMNSLRAEDTAVYFCARHMQVGGAPTGSMAAFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 219 aa, synthetic construct
  REGION 1..219: note = Description of Artificial Sequence: Synthetic polypeptide
DAQLTQSPSSLSASVGDRVTITCQSSQSVYNNNYLSWFQQKPGKPPKLLIYGASTLTSGVPSRFSGSGSGTDFTLTISSLQPEDFATYYCAGGYSGSSDKYAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 455 aa, synthetic construct
  REGION 1..455: note = Description of Artificial Sequence: Synthetic polypeptide
EQQLQESGPGLVKPSETLSLTCTVTGFSLSSYTVNWVRQPAGKGLEWIGYISYGGSAYYASWANGRFTISKDSSKNQVSLKLSSVTAADTAVYFCARHMQVGGAPTGSMAAFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 219 aa, synthetic construct
  REGION 1..219: note = Description of Artificial Sequence: Synthetic polypeptide
EAVLTQSPGTLSLSPGERATLSCQSSQSVYNNNYLSWFQQKPGQPPRLLIYGASTLTSGVPDRFSGSGSGTDFTLTISRLEPEDFAVYYCAGGYSGSSDKYAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 219 aa, synthetic construct
  REGION 1..219: note = Description of Artificial Sequence: Synthetic polypeptide
DAVLTQTPLSSPVTLGQPASISCQSSQSVYNNNYLSWFQQRPGQPPRLLIYGASTLTSGVPDRFSGSGAGTDFTLKISRVEAEDVGVYYCAGGYSGSSDKYAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 454 aa, synthetic construct
  REGION 1..454: note = Description of Artificial Sequence: Synthetic polypeptide
QQLQESGPGLVKPSETLSLTCTVTGFSLSSYTVNWVRQPAGKGLEWIGYISYGGSAYYASWANGRFTISKDSSKNQVSLKLSSVTAADTAVYFCARHMQVGGAPTGSMAAFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 453 aa, synthetic construct
  REGION 1..453: note = Description of Artificial Sequence: Synthetic polypeptide
EQQLQESGPGLVKPSETLSLTCTVTGFSLSSYTVNWVRQPAGKGLEWIGYISYGGSAYYASWANGRFTISKDSSQVSLKLSSVTAADTAVYFCARHMQVGGAPTGSMAAFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 452 aa, synthetic construct
  REGION 1..452: note = Description of Artificial Sequence: Synthetic polypeptide
QQLQESGPGLVKPSETLSLTCTVTGFSLSSYTVNWVRQPAGKGLEWIGYISYGGSAYYASWANGRFTISKDSSQVSLKLSSVTAADTAVYFCARHMQVGGAPTGSMAAFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 219 aa, synthetic construct
  REGION 1..219: note = Description of Artificial Sequence: Synthetic polypeptide
EIVLTQSPGTLSLSPGERATLSCQSSQSVYNNNYLSWFQQKPGQPPRLLIYGASTLTSGVPDRFSGSGSGTDFTLTISRLEPEDFAVYYCAGGYSGSSDKYAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 219 aa, synthetic construct
  REGION 1..219: note = Description of Artificial Sequence: Synthetic polypeptide
EAVLTQSPGTLSLSPGERATLSCQSSQSVYNNNYLSWYQQKPGQPPRLLIYGASTLTSGVPDRFSGSGSGTDFTLTISRLEPEDFAVYYCAGGYSGSSDKYAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 219 aa, synthetic construct
  REGION 1..219: note = Description of Artificial Sequence: Synthetic polypeptide
EAVLTQSPGTLSLSPGERATLSCQSSQSVYNNNYLSWFQQKPGQAPRLLIYGASTLTSGVPDRFSGSGSGTDFTLTISRLEPEDFAVYYCAGGYSGSSDKYAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 219 aa, synthetic construct
  REGION 1..219: note = Description of Artificial Sequence: Synthetic polypeptide
EAVLTQSPGTLSLSPGERATLSCQSSQSVYNNNYLSWFQQKPGQPPRLLIYGASTLTSGIPDRFSGSGSGTDFTLTISRLEPEDFAVYYCAGGYSGSSDKYAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 455 aa, synthetic construct
  REGION 1..455: note = Description of Artificial Sequence: Synthetic polypeptide
EVQLQESGPGLVKPSETLSLTCTVTGFSLSSYTVNWVRQPAGKGLEWIGYISYGGSAYYASWANGRFTISKDSSKNQVSLKLSSVTAADTAVYFCARHMQVGGAPTGSMAAFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 455 aa, synthetic construct
  REGION 1..455: note = Description of Artificial Sequence: Synthetic polypeptide
EQQLQESGPGLVKPSETLSLTCTVTGFSLSSYTVNWIRQPAGKGLEWIGYISYGGSAYYASWANGRFTISKDSSKNQVSLKLSSVTAADTAVYFCARHMQVGGAPTGSMAAFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 455 aa, synthetic construct
  REGION 1..455: note = Description of Artificial Sequence: Synthetic polypeptide
EQQLQESGPGLVKPSETLSLTCTVTGFSLSSYTVNWVRQPAGKGLEWIGYISYGGSAYYASWANGRVTISKDSSKNQVSLKLSSVTAADTAVYFCARHMQVGGAPTGSMAAFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 455 aa, synthetic construct
  REGION 1..455: note = Description of Artificial Sequence: Synthetic polypeptide
EQQLQESGPGLVKPSETLSLTCTVTGFSLSSYTVNWVRQPAGKGLEWIGYISYGGSAYYASWANGRFTISVDSSKNQVSLKLSSVTAADTAVYFCARHMQVGGAPTGSMAAFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 455 aa, synthetic construct
  REGION 1..455: note = Description of Artificial Sequence: Synthetic polypeptide
EQQLQESGPGLVKPSETLSLTCTVTGFSLSSYTVNWVRQPAGKGLEWIGYISYGGSAYYASWANGRFTISKDTSKNQVSLKLSSVTAADTAVYFCARHMQVGGAPTGSMAAFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 455 aa, synthetic construct
  REGION 1..455: note = Description of Artificial Sequence: Synthetic polypeptide
EQQLQESGPGLVKPSETLSLTCTVTGFSLSSYTVNWVRQPAGKGLEWIGYISYGGSAYYASWANGRFTISKDSSKNQFSLKLSSVTAADTAVYFCARHMQVGGAPTGSMAAFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 455 aa, synthetic construct
  REGION 1..455: note = Description of Artificial Sequence: Synthetic polypeptide
EQQLQESGPGLVKPSETLSLTCTVTGFSLSSYTVNWVRQPAGKGLEWIGYISYGGSAYYASWANGRFTISKDSSKNQVSLKLSSVTAADTAVYYCARHMQVGGAPTGSMAAFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 455 aa, synthetic construct
  REGION 1..455: note = Description of Artificial Sequence: Synthetic polypeptide
EQQLQESGPGLVKPSETLSLTCTVTGFSLSSYTVNWVRQPAGKGLEWIGYISYGGSAYYASWANGRFTISKDSSKNQVSLKLSSVTAADTAVYFCARHMQVGGAPTGSMAAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 455 aa, synthetic construct
  REGION 1..455: note = Description of Artificial Sequence: Synthetic polypeptide
EVQLQESGPGLVKPSETLSLTCTVTGFSLSSYTVNWIRQPAGKGLEWIGYISYGGSAYYASWANGRVTISKDTSKNQFSLKLSSVTAADTAVYFCARHMQVGGAPTGSMAAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 455 aa, synthetic construct
  REGION 1..455: note = Description of Artificial Sequence: Synthetic polypeptide
EVQLQESGPGLVKPSETLSLTCTVTGFSLSSYTVNWIRQPAGKGLEWIGYISYGGSAYYASWANGRVTISKDTSKNQVSLKLSSVTAADTAVYYCARHMQVGGAPTGSMAAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 455 aa, synthetic construct
  REGION 1..455: note = Description of Artificial Sequence: Synthetic polypeptide
EVQLQESGPGLVKPSETLSLTCTVTGFSLSSYTVNWIRQPAGKGLEWIGYISYGGSAYYASWANGRVTISKDTSKNQVSLKLSSVTAADTAVYFCARHMQVGGAPTGSMAAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 453 aa, synthetic construct
  REGION 1..453: note = Description of Artificial Sequence: Synthetic polypeptide
EVQLQESGPGLVKPSETLSLTCTVTGFSLSSYTVNWIRQPAGKGLEWIGYISYGGSAYYASWANGRVTISKDTSQVSLKLSSVTAADTAVYFCARHMQVGGAPTGSMAAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 107 aa, synthetic construct
  REGION 1..107: note = Description of Artificial Sequence: Synthetic polypeptide
DIQMTQSPSTLSASVGDRVTITCLASEDIYSNLAWYQQKPGKSPKLLIYDARSLQDGVPSRFSGSESGPEYTLTISSLQPDDFATYFCQQHHAYPFTFGQGTKVEIK

SEQ ID NO: 132 - protein, 119 aa, synthetic construct
  REGION 1..119: note = Description of Artificial Sequence: Synthetic polypeptide
EVTLKESGPVLVKPTETLTLTCTVSGFSLTTYNVHWVRQPPGKALEWMGLIWNTGGTRYNSALKSRLTISKDTSKSQVVLTMTNMDPVDTATYYCARDPVPNKWHFDFWGPGTLVTVSS

SEQ ID NO: 133 - protein, 107 aa, synthetic construct
  REGION 1..107: note = Description of Artificial Sequence: Synthetic polypeptide
DIQMTQSPSTLSASVGDRVTITCLASEDIYSNLAWYQQKPGKAPKLLIYDARSLQDGVPSRFSGSESGPEYTLTISSLQPDDFATYFCQQHHAYPFTFGQGTKVEIK

SEQ ID NO: 134 - protein, 107 aa, synthetic construct
  REGION 1..107: note = Description of Artificial Sequence: Synthetic polypeptide
DIQMTQSPSTLSASVGDRVTITCLASEDIYSNLAWYQQKPGKSPKLLIYDARSLQDGVPSRFSGSGSGPEYTLTISSLQPDDFATYFCQQHHAYPFTFGQGTKVEIK

SEQ ID NO: 135 - protein, 107 aa, synthetic construct
  REGION 1..107: note = Description of Artificial Sequence: Synthetic polypeptide
DIQMTQSPSTLSASVGDRVTITCLASEDIYSNLAWYQQKPGKSPKLLIYDARSLQDGVPSRFSGSESGTEYTLTISSLQPDDFATYFCQQHHAYPFTFGQGTKVEIK

SEQ ID NO: 136 - protein, 107 aa, synthetic construct
  REGION 1..107: note = Description of Artificial Sequence: Synthetic polypeptide
DIQMTQSPSTLSASVGDRVTITCLASEDIYSNLAWYQQKPGKSPKLLIYDARSLQDGVPSRFSGSESGPEFTLTISSLQPDDFATYFCQQHHAYPFTFGQGTKVEIK

SEQ ID NO: 137 - protein, 107 aa, synthetic construct
  REGION 1..107: note = Description of Artificial Sequence: Synthetic polypeptide
DIQMTQSPSTLSASVGDRVTITCLASEDIYSNLAWYQQKPGKSPKLLIYDARSLQDGVPSRFSGSESGPEYTLTISSLQPDDFATYYCQQHHAYPFTFGQGTKVEIK

SEQ ID NO: 138 - protein, 119 aa, synthetic construct
  REGION 1..119: note = Description of Artificial Sequence: Synthetic polypeptide
EVTLKESGPVLVKPTETLTLTCTVSGFSLTTYNVHWIRQPPGKALEWMGLIWNTGGTRYNSALKSRLTISKDTSKSQVVLTMTNMDPVDTATYYCARDPVPNKWHFDFWGPGTLVTVSS

SEQ ID NO: 139 - protein, 119 aa, synthetic construct
  REGION 1..119: note = Description of Artificial Sequence: Synthetic polypeptide
EVTLKESGPVLVKPTETLTLTCTVSGFSLTTYNVHWVRQPPGKALEWLGLIWNTGGTRYNSALKSRLTISKDTSKSQVVLTMTNMDPVDTATYYCARDPVPNKWHFDFWGPGTLVTVSS

SEQ ID NO: 140 - protein, 119 aa, synthetic construct
  REGION 1..119: note = Description of Artificial Sequence: Synthetic polypeptide
EVTLKESGPVLVKPTETLTLTCTVSGFSLTTYNVHWVRQPPGKALEWMALIWNTGGTRYNSALKSRLTISKDTSKSQVVLTMTNMDPVDTATYYCARDPVPNKWHFDFWGPGTLVTVSS

SEQ ID NO: 141 - protein, 119 aa, synthetic construct
  REGION 1..119: note = Description of Artificial Sequence: Synthetic polypeptide
EVTLKESGPVLVKPTETLTLTCTVSGFSLTTYNVHWVRQPPGKALEWMGLIWNTGGTRYNSALKSRLTISKDTSKSQVVLTMTNMDPVDTATYYCARDPVPNKWHFDFWGRGTLVTVSS

SEQ ID NO: 142 - protein, 119 aa, synthetic construct
  REGION 1..119: note = Description of Artificial Sequence: Synthetic polypeptide
EVQLQESGPGLVKPSETLSLTCTVSGFSLTTYNVHWVRQPPGKGLEWMGLIWNTGGTRYNSALKSRLTISKDTSKSQVSLKLSSVTAADTAVYYCARDPVPNKWHFDFWGPGTLVTVSS

SEQ ID NO: 143 - protein, 107 aa, synthetic construct
  REGION 1..107: note = Description of Artificial Sequence: Synthetic polypeptide
EIVMTQSPATLSVSPGERATLSCLASEDIYSNLAWYQQKPGQSPRLLIYDARSLQDGVPARFSGSESGPEYTLTISSLQSEDFAVYFCQQHHAYPFTFGQGTKVEIK

SEQ ID NO: 144 - protein, 107 aa, synthetic construct; identical to SEQ ID NO: 143
  REGION 1..107: note = Description of Artificial Sequence: Synthetic polypeptide

SEQ ID NO: 145 - protein, 214 aa, synthetic construct
  REGION 1..214: note = Description of Artificial Sequence: Synthetic polypeptide
DIQMTQSPSTLSASVGDRVTITCLASEDIYSNLAWYQQKPGKSPKLLIYDARSLQDGVPSRFSGSESGPEYTLTISSLQPDDFATYFCQQHHAYPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 449 aa, synthetic construct
  REGION 1..449: note = Description of Artificial Sequence: Synthetic polypeptide
EVTLKESGPVLVKPTETLTLTCTVSGFSLTTYNVHWVRQPPGKALEWMGLIWNTGGTRYNSALKSRLTISKDTSKSQVVLTMTNMDPVDTATYYCARDPVPNKWHFDFWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 214 aa, synthetic construct
  REGION 1..214: note = Description of Artificial Sequence: Synthetic polypeptide
DIQMTQSPSTLSASVGDRVTITCLASEDIYSNLAWYQQKPGKAPKLLIYDARSLQDGVPSRFSGSESGPEYTLTISSLQPDDFATYFCQQHHAYPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - protein, 214 aa, synthetic construct
  REGION 1..214: note = Description of Artificial Sequence: Synthetic polypeptide
DIQMTQSPSTLSASVGDRVTITCLASEDIYSNLAWYQQKPGKSPKLLIYDARSLQDGVPSRFSGSGSGPEYTLTISSLQPDDFATYFCQQHHAYPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 149 - protein, 214 aa, synthetic construct
  REGION 1..214: note = Description of Artificial Sequence: Synthetic polypeptide
DIQMTQSPSTLSASVGDRVTITCLASEDIYSNLAWYQQKPGKSPKLLIYDARSLQDGVPSRFSGSESGTEYTLTISSLQPDDFATYFCQQHHAYPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 214 aa, synthetic construct
  REGION 1..214: note = Description of Artificial Sequence: Synthetic polypeptide
DIQMTQSPSTLSASVGDRVTITCLASEDIYSNLAWYQQKPGKSPKLLIYDARSLQDGVPSRFSGSESGPEFTLTISSLQPDDFATYFCQQHHAYPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - protein, 214 aa, synthetic construct
  REGION 1..214: note = Description of Artificial Sequence: Synthetic polypeptide
DIQMTQSPSTLSASVGDRVTITCLASEDIYSNLAWYQQKPGKSPKLLIYDARSLQDGVPSRFSGSESGPEYTLTISSLQPDDFATYYCQQHHAYPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 449 aa, synthetic construct
  REGION 1..449: note = Description of Artificial Sequence: Synthetic polypeptide
EVTLKESGPVLVKPTETLTLTCTVSGFSLTTYNVHWIRQPPGKALEWMGLIWNTGGTRYNSALKSRLTISKDTSKSQVVLTMTNMDPVDTATYYCARDPVPNKWHFDFWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 449 aa, synthetic construct
  REGION 1..449: note = Description of Artificial Sequence: Synthetic polypeptide
EVTLKESGPVLVKPTETLTLTCTVSGFSLTTYNVHWVRQPPGKALEWLGLIWNTGGTRYNSALKSRLTISKDTSKSQVVLTMTNMDPVDTATYYCARDPVPNKWHFDFWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 449 aa, synthetic construct
  REGION 1..449: note = Description of Artificial Sequence: Synthetic polypeptide
EVTLKESGPVLVKPTETLTLTCTVSGFSLTTYNVHWVRQPPGKALEWMALIWNTGGTRYNSALKSRLTISKDTSKSQVVLTMTNMDPVDTATYYCARDPVPNKWHFDFWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 214 aa, synthetic construct; identical to SEQ ID NO: 145
  REGION 1..214: note = Description of Artificial Sequence: Synthetic polypeptide

SEQ ID NO: 156 - protein, 449 aa, synthetic construct
  REGION 1..449: note = Description of Artificial Sequence: Synthetic polypeptide
EVQLQESGPGLVKPSETLSLTCTVSGFSLTTYNVHWVRQPPGKGLEWMGLIWNTGGTRYNSALKSRLTISKDTSKSQVSLKLSSVTAADTAVYYCARDPVPNKWHFDFWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 214 aa, synthetic construct
  REGION 1..214: note = Description of Artificial Sequence: Synthetic polypeptide
EIVMTQSPATLSVSPGERATLSCLASEDIYSNLAWYQQKPGQSPRLLIYDARSLQDGVPARFSGSESGPEYTLTISSLQSEDFAVYFCQQHHAYPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 214 aa, synthetic construct; identical to SEQ ID NO: 157
  REGION 1..214: note = Description of Artificial Sequence: Synthetic polypeptide

SEQ ID NO: 159 - protein, 19 aa, synthetic construct
  REGION 1..19: note = Description of Artificial Sequence: Synthetic peptide
DIVSKTYQYTTYYSDSVKD

SEQ ID NO: 160 - protein, 121 aa, synthetic construct
  REGION 1..121: note = Description of Artificial Sequence: Synthetic polypeptide
QVQLQQSGPELVRPGASVKLSCKASGYIFITYWMNWVKQRPGQGLEWIGQIFPASGSTNYNEMFEGKATLTVDTSSSTAYMQLSSLTSEDSAVYYCARGDGNYALDAMDYWGQGTSVTVSS

SEQ ID NO: 161 - protein, 111 aa, synthetic construct
  REGION 1..111: note = Description of Artificial Sequence: Synthetic polypeptide
DIVLTQSPASLAVSLGQRATISCRASESVDSYGNSFMHWYQQKSGQPPKLLIYLASNLESGVPARFSGSGSRTDFTLTIDPVEADDAATYYCQQNNEDPLTFGAGTKLEIK

SEQ ID NO: 162 - protein, 836 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYTTKIITKVVEPKIKVIEGSLQPIIKTEGPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 163 - protein, 757 aa, Homo sapiens
MVPDTACVLLLTLAALGASGQGQSPLGSDLGPQMLRELQETNAALQDVRELLRQQVREITFLKNTVMECDACGMQQSVRTGLPSVRPLLHCAPGFCFPGVACIQTESGARCGPCPAGFTGNGSHCTDVNECNAHPCFPRVRCINTSPGFRCEACPPGYSGPTHQGVGLAFAKANKQVCTDINECETGQHNCVPNSVCINTRGSFQCGPCQPGFVGDQASGCQRRAQRFCPDGSPSECHEHADCVLERDGSRSCVCAVGWAGNGILCGRDTDLDGFPDEKLRCPERQCRKDNCVTVPNSGQEDVDRDGIGDACDPDADGDGVPNEKDNCPLVRNPDQRNTDEDKWGDACDNCRSQKNDDQKDTDQDGRGDACDDDIDGDRIRNQADNCPRVPNSDQKDSDGDGIGDACDNCPQKSNPDQADVDHDFVGDACDSDQDQDGDGHQDSRDNCPTVPNSAQEDSDHDGQGDACDDDDDNDGVPDSRDNCRLVPNPGQEDADRDGVGDVCQDDFDADKVVDKIDVCPENAEVTLTDFRAFQTVVLDPEGDAQIDPNWVVLNQGREIVQTMNSDPGLAVGYTAFNGVDFEGTFHVNTVTDDDYAGFIFGYQDSSSFYVVMWKQMEQTYWQANPFRAVAEPGIQLKAVKSSTGPGEQLRNALWHTGDTESQVRLLWKDPRNVGWKDKKSYRWFLQHRPQVGYIRVRFYEGPELVADSNVVLDTTMRGGRLGVFCFSQENIIWANLRYRCNDTIPEDYETHQLRQA

SEQ ID NO: 164 - DNA, 3713 bp, Homo sapiens
  source 1..3713: mol_type = other DNA
gcactcggctgtgcggcggggcaggcatgggagccgcgcgctctctcccggcgcccacacctgtctgagcggcgcagcgagccgcggcccgggcgggctgctcggcgcggaacagtgctcggcatggcagggattccagggctcctcttccttctcttctttctgctctgtgctgttgggcaagtgagcccttacagtgccccctggaaacccacttggcctgcataccgcctccctgtcgtcttgccccagtctaccctcaatttagccaagccagactttggagccgaagccaaattagaagtatcttcttcatgtggaccccagtgtcataagggaactccactgcccacttacgaagaggccaagcaatatctgtcttatgaaacgctctatgccaatggcagccgcacagagacgcaggtgggcatctacatcctcagcagtagtggagatggggcccaacaccgagactcagggtcttcaggaaagtctcgaaggaagcggcagatttatggctatgacagcaggttcagcatttttgggaaggacttcctgctcaactaccctttctcaacatcagtgaagttatccacgggctgcaccggcaccctggtggcagagaagcatgtcctcacagctgcccactgcatacacgatggaaaaacctatgtgaaaggaacccagaagcttcgagtgggcttcctaaagcccaagtttaaagatggtggtcgaggggccaacgactccacttcagccatgcccgagcagatgaaatttcagtggatccgggtgaaacgcacccatgtgcccaagggttggatcaagggcaatgccaatgacatcggcatggattatgattatgccctcctggaactcaaaaagccccacaagagaaaatttatgaagattggggtgagccctcctgctaagcagctgccagggggcagaattcacttctctggttatgacaatgaccgaccaggcaatttggtgtatcgcttctgtgacgtcaaagacgagacctatgacttgctctaccagcaatgcgatgcccagccaggggccagcgggtctggggtctatgtgaggatgtggaagagacagcagcagaagtgggagcgaaaaattattggcattttttcagggcaccagtgggtggacatgaatggttccccacaggatttcaacgtggctgtcagaatcactcctctcaaatatgcccagatttgctattggattaaaggaaactacctggattgtagggaggggtgacacagtgttccctcctggcagcaattaagggtcttcatgttcttattttaggagaggccaaattgttttttgtcattggcgtgcacacgtgtgtgtgtgtgtgtgtgtgtgtgtaaggtgtcttataatcttttacctatttcttacaattgcaagatgactggctttactatttgaaaactggtttgtgtatcatatcatatatcatttaagcagtttgaaggcatacttttgcatagaaataaaaaaaatactgatttggggcaatgaggaatatttgacaattaagttaatcttcacgtttttgcaaactttgatttttatttcatctgaacttgtttcaaagatttatattaaatatttggcatacaagagatatgaattcttatatgtgtgcatgtgtgttttcttctgagattcatcttggtggtgggtttttttgtttttttaattcagtgcctgatctttaatgcttccataaggcagtgttcccatttaggaactttgacagcatttgttaggcagaatattttggatttggaggcatttgcatggtagtctttgaacagtaaaatgatgtgttgactatactgatacacatattaaactataccttatagtaaaccagtatcccaagctgcttttagttccaaaaatagtttcttttccaaaggttgttgctctactttgtaggaagtctttgcatatggccctcccaactttaaagtcataccagagtggccaagagtgtttatcccaacccttccatttaacaggatttcactcacatttctggaactagctatttttcagaagacaataatcagggcttaattagaacaggctgtatttcctcccagcaaacagttgtggccacactaaaaacaatcatagcattttacccctggattatagcacatctcatgttttatcatttggatggagtaatttaaaatgaattaaattccagagaacaatggaagcattgcctggcagatgtcacaacagaataaccacttgtttggagcctggcacagtcctccagcctgatcaaaaattattctgcatagttttcagtgtgctttctgggagctatgtacttcttcaatttggaaacttttctctctcatttatagtgaaaatacttggaagttactttaagaaaaccagtgtggcctttttccctctagctttaaaagggccgcttttgctggaatgctctaggttatagataaacaattaggtataatagcaaaaatgaaaattggaagaatgcaaaatggatcagaatcatgccttccaataaaggcctttacacatgttttatcaatatgattatcaaatcacagcatatacagaaaagacttggacttattgtatgtttttattttatggctctcggcctaagcacttctttctaaatgtatcggagaaaaaatcaaatggactacaagcacgtgtttgctgtgcttgcaccccaggtaaacctgcattgtagcaatttgtaaggatattcagatggagcactgtcacttagacattctctgggggattttctgcttgtctttcttgagctttttggaaggataattctgataaggcactcaagaaacgtacaaccacagtgctttcttcaaatcatatgagaaatactatgcatagcaaggagatgcagagccgccaggaaaattctgagttccagcacaattttctttggaatctaacaggaatctagcctgaggaagaagggaggtctccatttctatgtctggtatttgggggttttgtttgtttttgctttagcttggtgaaaaaaagttcactgaacaccaagaccagaatggatttttttaaaaaaatagatgttccttttgtgaagcaccttgattccttgattttgattttttgcaaagttagacaatggcacaaagtcaaaatgaaatcaatgtttagttcacaagtagatgtaatttactaaagaatgatacacccatatgctatatacagcttaactcacagaactgtaaaagaaaattataaaataattcaacatgtccatctttttagtgataataaaagaaagcatggtattaaactatcatagaagtagacagaaaaagaaaaaaggactcatggcattattaatataattagtgctttacatgtgttagttatacatattagaagcatatttgcctagtaaggctagtagaaccacatttcccaaagtgtgctccttaaacactcatgccttatgattttctaccaaaagtaaaaagggttgtattaagtcagaggaagatgcctctccattttccctctctttatcagaggttcacatgcctgtctgcacattaaaagctctgggaagacctgttgtaaagggacaagttgaggttgtaaaatctgcatttaaataaacatctttgatcacaaaa

SEQ ID NO: 165 - DNA, 6817 bp, Homo sapiens
  source 1..6817: mol_type = other DNA
gcagtcgggagccggccgtggtggctccgtgcgtccgagcgtccgtccgcgccgtcggccatggccaagcgctccaggggccccgggcgccgctgcctgttggcgctcgtgctgttctgcgcctgggggacgctggccgtggtggcccagaagccgggcgcagggtgtccgagccgctgcctgtgcttccgcaccaccgtgcgctgcatgcatctgctgctggaggccgtgcccgccgtggcgccgcagacctccatcctagatcttcgctttaacagaatcagagagatccaacctggggcattcaggcggctgaggaacttgaacacattgcttctcaataataatcagatcaagaggatacctagtggagcatttgaagacttggaaaatttaaaatatctctatctgtacaagaatgagatccagtcaattgacaggcaagcatttaagggacttgcctctctagagcaactatacctgcactttaatcagatagaaactttggacccagattcgttccagcatctcccgaagctcgagaggctatttttgcataacaaccggattacacatttagttccagggacatttaatcacttggaatctatgaagagattgcgactggactcaaacacacttcactgcgactgtgaaatcctgtggttggcggatttgctgaaaacctacgcggagtcggggaacgcgcaggcagcggccatctgtgaatatcccagacgcatccagggacgctcagtggcaaccatcaccccggaagagctgaactgtgaaaggccccggatcacctccgagccccaggacgcagatgtgacctcggggaacaccgtgtacttcacctgcagagccgaaggcaaccccaagcctgagatcatctggctgcgaaacaataatgagctgagcatgaagacagattcccgcctaaacttgctggacgatgggaccctgatgatccagaacacacaggagacagaccagggtatctaccagtgcatggcaaagaacgtggccggagaggtgaagacgcaagaggtgaccctcaggtacttcgggtctccagctcgacccacttttgtaatccagccacagaatacagaggtgctggttggggagagcgtcacgctggagtgcagcgccacaggccaccccccgccgcggatctcctggacgagaggtgaccgcacacccttgccagttgacccgcgggtgaacatcacgccttctggcgggctttacatacagaacgtcgtacagggggacagcggagagtatgcgtgctctgcgaccaacaacattgacagcgtccatgccaccgctttcatcatcgtccaggctcttcctcagttcactgtgacgcctcaggacagagtcgttattgagggccagaccgtggatttccagtgtgaagccaagggcaacccgccgcccgtcatcgcctggaccaagggagggagccagctctccgtggaccggcggcacctggtcctgtcatcgggaacacttagaatctctggtgttgccctccacgaccagggccagtacgaatgccaggctgtcaacatcatcggctcccagaaggtcgtggcccacctgactgtgcagcccagagtcaccccagtgtttgccagcattcccagcgacacaacagtggaggtgggcgccaatgtgcagctcccgtgcagctcccagggcgagcccgagccagccatcacctggaacaaggatggggttcaggtgacagaaagtggaaaatttcacatcagccctgaaggattcttgaccatcaatgacgttggccctgcagacgcaggtcgctatgagtgtgtggcccggaacaccattgggtcggcctcggtgagcatggtgctcagtgtgaatgttcctgacgtcagtcgaaatggagatccgtttgtagctacctccatcgtggaagcgattgcgactgttgacagagctataaactcaacccgaacacatttgtttgacagccgtcctcgttctccaaatgatttgctggccttgttccggtatccgagggatccttacacagttgaacaggcacgggcgggagaaatctttgaacggacattgcagctcattcaggagcatgtacagcatggcttgatggtcgacctcaacggaacaagttaccactacaacgacctggtgtctccacagtacctgaacctcatcgcaaacctgtcgggctgtaccgcccaccggcgcgtgaacaactgctcggacatgtgcttccaccagaagtaccggacgcacgacggcacctgtaacaacctgcagcaccccatgtggggcgcctcgctgaccgccttcgagcgcctgctgaaatccgtgtacgagaatggcttcaacacccctcggggcatcaacccccaccgactgtacaacgggcacgcccttcccatgccgcgcctggtgtccaccaccctgatcgggacggagaccgtcacacccgacgagcagttcacccacatgctgatgcagtggggccagttcctggaccacgacctcgactccacggtggtggccctgagccaggcacgcttctccgacggacagcactgcagcaacgtgtgcagcaacgaccccccctgcttctctgtcatgatcccccccaatgactcccgggccaggagcggggcccgctgcatgttcttcgtgcgctccagccctgtgtgcggcagcggcatgacttcgctgctcatgaactccgtgtacccgcgggagcagatcaaccagctcacctcctacatagacgcatccaacgtgtacgggagcacggagcatgaggcccgcagcatccgcgacctggccagccaccgcggcctgctgcggcagggcatcgtgcagcggtccgggaagccgctgctccccttcgccaccgggccgcccacggagtgcatgcgggacgagaacgagagccccatcccctgcttcctggccggggaccaccgcgccaacgagcagctgggcctgaccagcatgcacacgctgtggttccgcgagcacaaccgcattgccacggagctgctcaagctgaacccgcactgggacggcgacaccatctactatgagaccaggaagatcgtgggtgcggagatccagcacatcacctaccagcactggctcccgaagatcctgggggaggtgggcatgaggacgctgggagagtaccacggctacgaccccggcatcaatgctggcatcttcaacgccttcgccaccgcggccttcaggtttggccacacgcttgtcaacccactgctttaccggctggacgagaacttccagcccattgcacaagatcacctcccccttcacaaagctttcttctctcccttccggattgtgaatgagggcggcatcgatccgcttctcagggggctgttcggggtggcggggaaaatgcgtgtgccctcgcagctgctgaacacggagctcacggagcggctgttctccatggcacacacggtggctctggacctggcggccatcaacatccagcggggccgggaccacgggatcccaccctaccacgactacagggtctactgcaatctatcggcggcacacacgttcgaggacctgaaaaatgagattaaaaaccctgagatccgggagaaactgaaaaggttgtatggctcgacactcaacatcgacctgtttccggcgctcgtggtggaggacctggtgcctggcagccggctgggccccaccctgatgtgtcttctcagcacacagttcaagcgcctgcgagatggggacaggttgtggtatgagaaccctggggtgttctccccggcccagctgactcagatcaagcagacgtcgctggccaggatcctatgcgacaacgcggacaacatcacccgggtgcagagcgacgtgttcagggtggcggagttccctcacggctacggcagctgtgacgagatccccagggtagacctccgggtgtggcaggactgctgtgaagactgtaggaccagggggcagttcaatgccttttcctatcatttccgaggcagacggtctcttgagttcagctaccaggaggacaagccgaccaagaaaacaagaccacggaaaatacccagtgttgggagacagggggaacatctcagcaacagcacctcagccttcagcacacgctcagatgcatctgggacaaatgacttcagagagtttgttctggaaatgcagaagaccatcacagacctcagaacacagataaagaaacttgaatcacggctcagtaccacagagtgcgtggatgccgggggcgaatctcacgccaacaacaccaagtggaaaaaagatgcatgcaccatttgtgaatgcaaagacgggcaggtcacctgcttcgtggaagcttgcccccctgccacctgtgctgtccccgtgaacatcccaggggcctgctgtccagtctgcttacagaagagggcggaggaaaagccctaggctcctgggaggctcctcagagtttgtctgctgtgccatcgtgagatcgggtggccgatggcagggagctgcggactgcagaccaggaaacacccagaactcgtgacatttcatgacaacgtccagctggtgctgttacagaaggcagtgcaggaggcttccaaccagagcatctgcggagaaggaggcacagcaggtgcctgaagggaagcaggcaggagtcctagcttcacgttagacttctcaggtttttatttaattcttttaaaatgaaaaattggtgctactattaaattgcacagttgaatcatttaggcgcctaaattgattttgcctcccaacaccatttctttttaaataaagcaggatacctctatatgtcagccttgccttgttcagatgccaggagccggcagacctgtcacccgcaggtggggtgagtcttggagctgccagaggggctcaccgaaatcggggttccatcacaagctatgtttaaaaagaaaattggtgtttggcaaacggaacagaacctttgatgagagcgttcacagggacactgtctgggggtgcagtgcaagcccccggcctcttccctgggaacctctgaactcctccttcctctgggctctctgtaacatttcaccacacgtcagcatctaatcccaagacaaacattcccgctgctcgaagcagctgtatagcctgtgactctccgtgtgtcagctccttccacacctgattagaacattcataagccacatttagaaacaggtttgctttcagctgtcacttgcacacatactgcctagttgtgaaccaaatgtgaaaaaacctccttcatcccattgtgtatctgatacctgccgagggccaagggtgtgtgttgacaacgccgctcccagccggccctggttgcgtccacgtcctgaacaagagccgcttccggatggctcttcccaagggaggaggagctcaagtgtcgggaactgtctaacttcaggttgtgtgagtgcgttaaaaaaaaaaaaaaaaaagaatccctatacctcatttgtatttttaaaatgcgtgatgttttatgaaattgtgtccattttttaggtattagatatggcagaaaaaccatttccactatgcaaagttcttttagacgtcagtgaaaatcaactctcatacctcatggtctctctttaattgaccaaaaccttccatttttctctaaatacaaagcgatctgtgttctgagcaacctttccccgaacacacagcttcagtgcagcacgctgacctgagtatccaccatgtgccaggcacagtgctgggcacacgaggcaccaaggtccgggccacctgcccgcagcaaggcccagctgaggtggtggagggagcccctgaggtcaggggccgtttcggttcagggtggcaggtgtccagcactggggtatggcgtcgaggcttccatggggtgggggaggccagcttccttctgacaggatgggcgcatacagtgcctggtgtgatttgtgcacaacccgtgttccaggtgcacatcctcccaaggagacacccagacccttccagcacgggccggccaagttgctgcggcggaggcagcatttcagctgtgaggaaggtcattggattcatgtgttttatctgtaaaaatggttgtcttaacttcttaacctcatattggtaagtgattgataaaaattggttggtgtttcatgacatgtggacttcttttgaaatagcaagtcaaatgtagtgaccaaattgtggaagagatttctgtcaaataggaaatgtgtaagttcgtctaaaagctgatggttatgtaagttgctcaggcactcagatgacagcagattctgggttctgggagtgttctgtgcctcttacatgccctggaggcctcatggtctcagtgctgaggcggcacacctgtagcacacctgcgtaatgtgcggtctgggccagtcacaaggaattgtgttgtctaagccaaagggggaagctgactgtgatttaccaaaaaaaattctgtaattcaaaccaaaatgtctgcggaatcaccagtttgatactctctgtaatcagaacagtgggcagtgcctgggtgaacgtgtctagcagccactgtgcgggatcgctgtaacaggagtggaatgtacatatttatttacttttctaactgctccaacagccaaatgccttttttatgaccattgtattcagttcattaccaaagaaatgtttgcactttgtaatgatgcctttcagttcaaataaatgggtcacattttcaaatgga

SEQ ID NO: 166 - DNA, 5577 bp, Homo sapiens
  source 1..5577: mol_type = other DNA
ggatcacagcccttccccgatcctctccgtgggagccagcgagcctctctccctgatcttacgtgctcaagggagctcacacgttcaccaactcacccttgaagtcatctcaagaacaaaagacaactgaaagaagctgttgtgaaggcagagcagcatctgctgaagagacagaaaccagccccagaggtgtcacaggaaggcaccagcaaggacattggtctttgatttgattcagcagtcctgtcaagtataaatgtgatggctgtgctgcctggccctctgcagctgctgggagtgctgcttaccatttccctgagttccatcaggctcattcaggctggtgcctactatgggatcaagccgctgccacctcaaattcctcctcagatgccaccacaaattccacaataccagcccctgggtcagcaagtacctcacatgcctttggccaaagatggccttgccatgggcaaggagatgccccacttgcagtatggcaaagagtatccacacctaccccaatatatgaaggaaattcaaccggcgccaagaatgggcaaggaagccgtacccaagaaaggcaaagaaataccattagccagtttacgaggggaacaaggtccccgtggagagcctggcccaagaggaccacctgggccccctggtttgccaggtcatgggatacctggaattaaaggaaaaccagggccacagggatatccaggagttggaaagccaggtatgcctggaatgccagggaagccaggagccatgggcatgcctggggcaaaaggagaaattggacagaaaggggaaattgggcctatggggatcccaggaccacaaggacctccagggcctcatggacttcctggcattgggaagccaggtgggccagggttaccagggcaaccaggaccaaagggtgatcgaggacccaaaggactaccaggacctcaaggccttcggggtcctaaaggagacaagggcttcgggatgccaggtgcgccaggtgtaaaggggcctccagggatgcacggccctcccggccctgttggactgccaggagtgggcaaaccaggagtgacaggcttccctgggccccagggccccctgggaaagccaggggctccaggagaacctgggccacaaggccctattggggtaccgggggttcaaggacctcctgggatacccggaattggaaagccaggccaggatgggatcccaggccagccaggatttccaggtggcaaaggggagcaaggactgccagggctaccaggacccccaggccttccagggattgggaaaccaggcttcccaggacccaaaggtgaccggggcatgggaggtgttcctggggctcttggaccaagaggggagaaaggaccaataggtgccccaggaatagggggtcctccaggagagccaggcctgcctggaatcccaggtcctatgggccctccaggtgctattggttttcctggacccaaaggagaaggtgggattgtagggccacaggggccaccaggtcccaagggtgagccagggcttcaaggcttcccaggaaagccaggtttccttggtgaagtagggcctcctggcatgaggggtttgccaggtcccatagggcccaagggggaagctgggcaaaaaggtgtaccaggactccctggtgttccagggcttctcggacctaagggagagccaggaatcccaggggatcagggtttacagggccccccaggtatcccagggattgggggccctagtggccccattggaccacctgggattccaggccccaaaggggagccgggcctcccagggccccctgggttccctggtatagggaaacccggagtggcaggacttcatggccccccagggaagcctggtgcccttggtcctcaaggccagcctggccttccaggacccccaggccctccaggacctccaggacccccagctgtgatgccccctacaccaccaccccagggagagtatctgccagatatggggctgggaattgatggcgtgaaacccccccatgcctacggggctaagaaaggcaagaatggagggccagcctatgagatgcctgcatttaccgccgagctaaccgcacctttcccaccggtgggggccccagtgaagtttaacaaactgctgtataacggcagacagaactacaacccgcagacaggcatcttcacctgtgaggtccctggtgtctactactttgcataccacgttcactgcaaggggggcaacgtgtgggttgctctattcaagaacaacgagcccgtgatgtacacgtacgacgagtacaaaaagggcttcctggaccaggcatctgggagtgcagtgctgctgctcaggcccggagaccgggtgttcctccagatgccctcagaacaggctgcaggactgtatgccgggcagtatgtccactcctccttttcaggatatttattgtatcccatgtaaaaacaaaaaaacaaaaaacaaagaaaagaaagagattttatagaagaaaatgacacaccaaaaaatccaaatgaaaaacataattgcttcaaaacacttacacagttggaaagttatatgtaagtgaaaatttggaccattgtgtacaaataaaaactaagatgcatgtttaatactccacacagcagcctgtaattgcgaatgatgggatagagttatgtatcaagtactgacacttggttgtacccactggaatcatattagctgttttatgttatatgcttccacagtaacctgcttattcagatcagtcaaaatatatcagtatgaaagatcatagctaatgaaaggcactcactcatattgtttactttaaaatatttataaatatgccttaaagaaatacaaatgataacaattacataccgtatttacttgcttaatttcctctgtatttgtgtagatactttgacatggaatatatggtggggagacccgtagtgttaccgccccagtgggagggggccctggggaccctggtaatgctttagtcaaagggatatctctcttgtatcagaggctgtgtcttttagtaacaggagtcctcgtcagaattgcgtgtctgttgtctctaaaagaatgggtgaaccaatcggcctttgtgaatttattcagtgccttctctgtaccaagcactgggtaaggcacttttgtggagcattagacagtaaccctcaaggagctagagaaccggatgggagacatgagcagtaattaactcacttgttccccagagtttctatttgttttgattttctttttctgtgacttattttcctattttctttcctccatgtaattttcactatggcccaactaatataaacacctggaaattacaaggaaaaaaaattcttcctctaataactttccaaatttgtggaatatttatttgtaatagcagttatcagttatgcttatatagcattaaaaattctcctcctttgactacacacacaaccacagtgtggttctaatcatggagatatcagtaatttttagtaactgaattttgaggacatttctctgtttagcatgtatgcaaactgatatgtaatctgaggttccaaagtcaatttttttcttttttttttgagatggagtcttactctgtcacccaggctggagtgcagtagcacgatcttggcttactgcaacctctacctcctaggttcaagcaattgtcctgtctcagcctcccgagtactgggactactgtcttgcgccaccatgcctggctaatttttgtatatttagtaaagatgggttttcgccatgttggccaggctggtctcaaactcctggcctcaagtgatgcacctgcctcagccttccaaagtgctgggattacaggcatgagccaccgtgcccggccaaagtcagctttcaaaatccaagccataattggtgaggggggagtttcagaattacatagaaaaattaatatttgaaaaaataattctgaaatttcgaatttaaaaacagatgtgctgcttctgggtgtaggtagtaaaagtataggaaaagaactgtttccttagaagcggactgtggaagggctatgtagaatgtcaaagggcaacaagagcctgtgtttttaatgtcatcctgtactcggcacaaatcaaaggccaatacaagtctgaaaagcagaaataaatatttttccaggtttttgcttgggcacatactaactgctttgggcattctaatctggtctccaaacaccaaagacccatttcgagcctgctattagcctgctgctgactctatcacttggagcaataatgtggggttatggtggtggaatcttgtatatttttgtcaaaaataaaaccatgagttaaggggatagatatagatggaaaaatacacaaataaatacggtatgaaaacacatggaaatgtgtctttgtcaaatctgaatcattattaccatcacaaaaattcttctcttggccaatatctcatttccctatatagtatacaagcaccatttcttctcaatttttaagaagagaaattagtccattaccacaggggttcttgtcactactaattatacaacaatcttttcccaacaaaaagatgtcctccacaacctttgttttcaaagcagacagcatctatgtggccaaatatactttgggttgttcttgaggatactggttttgggctgatgactatggtgggcagcatggatccattgggctccttctgctaaacagccacattgaaatggtttaaaagcaagtcagatcaggtgatttgtaaaattgtatttatctgtacatgtatgggcttttaattcccaccaagaaagagagaaattatctttttagttaaaaccaaatttcacttttcaaaatatcttccaacttatttattggttgtcactcaattgcctatatatatatatatatgtgtgtgtgtgtgtgtgtgcgcgtgagcgcacgtgtgtgtatgcgtgcgcatgtgtgtgtatgtgtattatcagacataggtttctaacttttagatagaagaggagcaacatctatgccaaatactgtgcattctacaatggtgctaatctcagacctaaatgatactccatttaatttaaaaaagagttttaaataattatctatgtgcctgtatttcccttttgagtgctgcacaacatgttaacatattagtgtaaaagcagatgaaacaaccacgtgttctaaagtctagggattgtgctataatccctatttagttcaaaattaaccagaattcttccatgtgaaatggaccaaactcatattattgttatgtaaatacagagttttaatgcagtatgacatcccacaggggaaaagaatgtctgtagtgggtgactgttatcaaatattttatagaatacaatgaacggtgaacagactggtaacttgtttgagttcccatgacagatttgagacttgtcaatagcaaatcatttttgtatttaaatttttgtactgatttgaaaaacatcattaaatatctttaaaagta

SEQ ID NO: 167 - DNA, 10532 bp, Homo sapiens
  source 1..10532: mol_type = other DNA
agtcttccaccaaaggccgttcagttctcctgggctccagcctcctgcaaggactgcaagagttttcctccgcagctctgagtctccacttttttggtggagaaaggctgcaaaaagaaaaagagacgcagtgagtgggaaaagtatgcatcctattcaaacctaattgaatcgaggagcccagggacacacgccttcaggtttgctcaggggttcatatttggtgcttagacaaattcaaaatgaggaaacatcggcacttgcccttagtggccgtcttttgcctctttctctcaggctttcctacaactcatgcccagcagcagcaagcagatgtcaaaaatggtgcggctgctgatataatatttctagtggattcctcttggaccattggagaggaacatttccaacttgttcgagagtttctatatgatgttgtaaaatccttagctgtgggagaaaatgatttccattttgctctggtccagttcaacggaaacccacataccgagttcctgttaaatacgtatcgtactaaacaagaagtcctttctcatatttccaacatgtcttatattgggggaaccaatcagactggaaaaggattagaatacataatgcaaagccacctcaccaaggctgctggaagccgggccggtgacggagtccctcaggttatcgtagtgttaactgatggacactcgaaggatggccttgctctgccctcagcggaacttaagtctgctgatgttaacgtgtttgcaattggagttgaggatgcagatgaaggagcgttaaaagaaatagcaagtgaaccgctcaatatgcatatgttcaacctagagaattttacctcacttcatgacatagtaggaaacttagtgtcctgtgtgcattcatccgtgagtccagaaagggctggggacacggaaacccttaaagacatcacagcacaagactctgctgacattattttccttattgatggatcaaacaacaccggaagtgtcaatttcgcagtcattctcgacttccttgtaaatctccttgagaaactcccaattggaactcagcagatccgagtgggggtggtccagtttagcgatgagcccagaaccatgttctccttggacacctactccaccaaggcccaggttctgggtgcagtgaaagccctcgggtttgctggtggggagttggccaatatcggcctcgcccttgatttcgtggtggagaaccacttcacccgggcagggggcagccgcgtggaggaaggggttccccaggtgctggtcctcataagtgccgggccttctagtgacgagattcgctacggggtggtagcactgaagcaggctagcgtgttctcattcggccttggagcccaggccgcctccagggcagagcttcagcacatagctaccgatgacaacttggtgtttactgtcccggaattccgtagctttggggacctccaggagaaattactgccgtacattgttggcgtggcccaaaggcacattgtcttgaaaccgccaaccattgtcacacaagtcattgaagtcaacaagagagacatagtcttcctggtggatggctcatctgcactgggactggccaacttcaatgccatccgagacttcattgctaaagtcatccagaggctggaaatcggacaggatcttatccaggtggcagtggcccagtatgcagacactgtgaggcctgaattttatttcaatacccatccaacaaaaagggaagtcataaccgctgtgcggaaaatgaagcccctggacggctcggccctgtacacgggctctgctctagactttgttcgtaacaacctattcacgagttcagccggctaccgggctgccgaggggattcctaagcttttggtgctgatcacaggtggtaagtccctagatgaaatcagccagcctgcccaggagctgaagagaagcagcataatggcctttgccattgggaacaagggtgccgatcaggctgagctggaagagatcgctttcgactcctccctggtgttcatcccagctgagttccgagccgccccattgcaaggcatgctgcctggcttgctggcacctctcaggaccctctctggaacccctgaagttcactcaaacaaaagggatatcatctttcttttggatggatcagccaacgttggaaaaaccaatttcccttatgtgcgcgactttgtaatgaacctagttaacagccttgatattggaaatgacaatattcgtgttggtttagtgcaatttagtgacactcctgtaacggagttctctttaaacacataccagaccaagtcagatatccttggtcatctgaggcagctgcagctccagggaggttcgggcctgaacacaggctcagccctaagctatgtctatgccaaccacttcacggaagctggcggcagcaggatccgtgaacacgtgccgcagctcctgcttctgctcacagctgggcagtctgaggactcctatttgcaagctgccaacgccttgacacgcgcgggcatcctgactttttgtgtgggagctagccaggcgaataaggcagagcttgagcagattgcttttaacccaagcctggtgtatctcatggatgatttcagctccctgccagctttgcctcagcagctgattcagcccctaaccacatatgttagtggaggtgtggaggaagtaccactcgctcagccagagagcaagcgagacattctgttcctctttgacggctcagccaatcttgtgggccagttccctgttgtccgtgactttctctacaagattatcgatgagctcaatgtgaagccagaggggacccgaattgcggtggctcagtacagcgatgatgtcaaggtggagtcccgttttgatgagcaccagagtaagcctgagatcctgaatcttgtgaagagaatgaagatcaagacgggcaaagccctcaacctgggctacgcgctggactatgcacagaggtacatttttgtgaagtctgctggcagccggatcgaggatggagtgcttcagttcctggtgctgctggtcgcaggaaggtcatctgaccgtgtggatgggccagcaagtaacctgaagcagagtggggttgtgcctttcatcttccaagccaagaacgcagaccctgctgagttagagcagatcgtgctgtctccagcgtttatcctggctgcagagtcgcttcccaagattggagatcttcatccacagatagtgaatctcttaaaatcagtgcacaacggagcaccagcaccagtttcaggtgaaaaggacgtggtgtttctgcttgatggctctgagggcgtcaggagcggcttccctctgttgaaagagtttgtccagagagtggtggaaagcctggatgtgggccaggaccgggtccgcgtggccgtggtgcagtacagcgaccggaccaggcccgagttctacctgaattcatacatgaacaagcaggacgtcgtcaacgctgtccgccagctgaccctgctgggagggccgacccccaacaccggggccgccctggagtttgtcctgaggaacatcctggtcagctctgcgggaagcaggataacagaaggtgtgccccagctgctgatcgtcctcacggccgacaggtctggggatgatgtgcggaacccctccgtggtcgtgaagaggggtggggctgtgcccattggcattggcatcgggaacgctgacatcacagagatgcagaccatctccttcatcccggactttgccgtggccattcccacctttcgccagctggggaccgtccaacaggtcatctctgagagggtgacccagctcacccgcgaggagctgagcaggctgcagccggtgttgcagcctctaccgagcccaggtgttggtggcaagagggacgtggtctttctcatcgatgggtcccaaagtgccgggcctgagttccagtacgttcgcaccctcatagagaggctggttgactacctggacgtgggctttgacaccacccgggtggctgtcatccagttcagcgatgaccccaaggtggagttcctgctgaacgcccattccagcaaggatgaagtgcagaacgcggtgcagcggctgaggcccaagggagggcggcagatcaacgtgggcaatgccctggagtacgtgtccaggaacatcttcaagaggcccctggggagccgcattgaagagggcgtcccgcagttcctggtcctcatctcgtctggaaagtctgacgatgaggtggacgacccggcggtggagctcaagcagtttggcgtggcccctttcacgatcgccaggaacgcagaccaggaggagctggtgaagatctcgctgagccccgaatatgtgttctcggtgagcaccttccgggagctgcccagcctggagcagaaactgctgacgcccatcacgaccctgacctcagagcagatccagaagctcttagccagcactcgctatccacctccagcagttgagagtgatgctgcagacattgtctttctgatcgacagctctgagggagttaggccagatggctttgcacatattcgagattttgttagcaggattgttcgaagactcaacatcggccccagtaaagtgagagttggggtcgtgcagttcagcaatgatgtcttcccagaattctatctgaaaacctacagatcccaggccccggtgctggacgccatacggcgcctgaggctcagaggggggtccccactgaacactggcaaggctctcgaatttgtggcaagaaacctctttgttaagtctgcggggagtcgcatagaagacggggtgccccaacacctggtcctggtcctgggtggaaaatcccaggacgatgtgtccaggttcgcccaggtgatccgttcctcgggcattgtgagtttaggggtaggagaccggaacatcgacagaacagagctgcagaccatcaccaatgaccccagactggtcttcacagtgcgagagttcagagagcttcccaacatagaagaaagaatcatgaactcgtttggaccctccgcagccactcctgcacctccaggggtggacacccctcctccttcacggccagagaagaagaaagcagacattgtgttcctgttggatggttccatcaacttcaggagggacagtttccaggaagtgcttcgttttgtgtctgaaatagtggacacagtttatgaagatggcgactccatccaagtggggcttgtccagtacaactctgaccccactgacgaattcttcctgaaggacttctctaccaagaggcagattattgacgccatcaacaaagtggtctacaaagggggaagacacgccaacactaaggtgggccttgagcacctgcgggtaaaccactttgtgcctgaggcaggcagccgcctggaccagcgggtccctcagattgcctttgtgatcacgggaggaaagtcggtggaagatgcacaggatgtgagcctggccctcacccagaggggggtcaaagtgtttgctgttggagtgaggaatatcgactcggaggaggttggaaagatagcgtccaacagcgccacagcgttccgcgtgggcaacgtccaggagctgtccgaactgagcgagcaagttttggaaactttgcatgatgcgatgcatgaaaccctttgccctggtgtaactgatgctgccaaagcttgtaatctggatgtgattctggggtttgatggttctagagaccagaatgtttttgtggcccagaagggcttcgagtccaaggtggacgccatcttgaacagaatcagccagatgcacagggtcagctgcagcggtggccgctcgcccaccgtgcgtgtgtcagtggtggccaacacgccctcgggcccggtggaggcctttgactttgacgagtaccagccagagatgctcgagaagttccggaacatgcgcagccagcacccctacgtcctcacggaggacaccctgaaggtctacctgaacaagttcagacagtcctcgccggacagcgtgaaggtggtcattcattttactgatggagcagacggagatctggctgatttacacagagcatctgagaacctccgccaagaaggagtccgtgccttgatcctggtgggccttgaacgagtggtcaacttggagcggctaatgcatctggagtttgggcgagggtttatgtatgacaggcccctgaggcttaacttgctggacttggattatgaactagcggagcagcttgacaacattgccgagaaagcttgctgtggggttccctgcaagtgctctgggcagaggggagaccgcgggcccatcggcagcatcgggccaaagggtattcctggagaagacggctaccgaggctatcctggtgatgagggtggacccggtgagcgtggtccgcctggtgtgaacggcactcaaggtttccagggctgcccgggccagagaggagtaaagggctctcggggattcccaggagagaagggcgaagtaggagaaattggactggatggtctggatggtgaagatggagacaaaggattgcctggttcttctggagagaaagggaatcctggaagaaggggtgataaaggacctcgaggagagaaaggagaaagaggagatgttgggattcgaggggacccgggtaacccaggacaagacagccaggagagaggacccaaaggagaaaccggtgacctcggccccatgggtgtcccagggagagatggagtacctggaggacctggagaaactgggaagaatggtggctttggccgaaggggaccccccggagctaagggcaacaagggcggtcctggccagccgggctttgagggagagcaggggaccagaggtgcacagggcccagctggtcctgctggtcctccagggctgataggagaacaaggcatttctggacctcggggaagcggaggtgccgctggtgctcctggagaacgaggcagaaccggtccactgggaagaaagggtgagcccggagagccaggaccaaaaggaggaatcgggaaccggggccctcgtggggagacgggagatgacgggagagacggagttggcagtgaaggacgcagaggcaaaaaaggagaaagaggattccctggatacccaggaccaaagggtaacccaggtgaacctgggctaaatggaacaacaggacccaaaggcatcagaggccgaaggggaaattcgggacctccagggatagttggacagaagggagaccctggctacccaggaccagctggtcccaagggcaacaggggcgactccatcgatcaatgtgccctcatccaaagcatcaaagataaatgcccttgctgttacgggcccctggagtgccccgtcttcccaacagaactagcctttgctttagacacctctgagggagtcaaccaagacactttcggccggatgcgagatgtggtcttgagtattgtgaatgacctgaccattgctgagagcaactgcccacggggggcccgggtggctgtggtcacctacaacaacgaggtgaccacggagatccggtttgctgactccaagaggaagtcggtcctcctggacaagattaagaaccttcaggtggctctgacatccaaacagcagagtctggagactgccatgtcgtttgtggccaggaacacatttaagcgtgtgaggaacggattcctaatgaggaaagtggctgttttcttcagcaacacacccacaagagcatccccacagctcagagaggctgtgctcaagctctcagatgcggggatcacccccttgttccttacaaggcaggaagaccggcagctcatcaacgctttgcagatcaataacacagcagtggggcatgcgcttgtcctgcctgcagggagagacctcacagacttcctggagaatgtcctcacgtgtcatgtttgcttggacatctgcaacatcgacccatcctgtggatttggcagttggaggccttccttcagggacaggagagcggcagggagcgatgtggacatcgacatggctttcatcttagacagcgctgagaccaccaccctgttccagttcaatgagatgaagaagtacatagcgtacctggtcagacaactggacatgagcccagatcccaaggcctcccagcacttcgccagagtggcagttgtgcagcacgcgccctctgagtccgtggacaatgccagcatgccacctgtgaaggtggaattctccctgactgactatggctccaaggagaagctggtggacttcctcagcaggggaatgacacagttgcagggaaccagggccttaggcagtgccattgaatacaccatagagaatgtctttgaaagtgccccaaacccacgggacctgaaaattgtggtcctgatgctgacgggcgaggtgccggagcagcagctggaggaggcccagagagtcatcctgcaggccaaatgcaagggctacttcttcgtggtcctgggcattggcaggaaggtgaacatcaaggaggtatacaccttcgccagtgagccaaacgacgtcttcttcaaattagtggacaagtccaccgagctcaacgaggagcctttgatgcgcttcgggaggctgttgccatccttcgtcagcagtgaaaatgctttttacttgtccccagatatcaggaaacagtgtgattggttccaaggggaccaacccacaaagaaccttgtgaagtttggtcacaaacaagtaaatgttccgaataacgttacttcaagtcctacatccaacccagtgacgacaacgaagccggtgactacgacgaagccggtgaccaccacaacaaagcctgtaaccaccacaacaaagcctgtgactattataaatcagccatctgtgaagccagccgctgcaaagccggcccctgcgaaacctgtggctgccaagcctgtggccacaaagatggccactgttagacccccagtggcggtgaagccagcaacggcagcgaagcctgtagcagcaaagccagcagctgtaagaccccccgctgctgctgctgcaaaaccagtggcgaccaagcctgaggtccctaggccacaggcagccaaaccagctgccaccaagccagccaccactaagcccatggttaagatgtcccgtgaagtccaggtgtttgagataacagagaacagcgccaaactccactgggagagggctgagccccccggtccttatttttatgacctcaccgtcacctcagcccatgatcagtccctggttctgaagcagaacctcacggtcacggaccgcgtcattggaggcctgctcgctgggcagacataccatgtggctgtggtctgctacctgaggtctcaggtcagagccacctaccacggaagtttcagtacaaagaaatctcagcccccacctccacagccagcaaggtcagcttctagttcaaccatcaatctaatggtgagcacagaaccattggctctcactgaaacagatatatgcaagttgccgaaagacgaaggaacttgcagggatttcatattaaaatggtactatgatccaaacaccaaaagctgtgcaagattctggtatggaggttgtggtggaaacgaaaacaaatttggatcacagaaagaatgtgaaaaggtttgcgctcctgtgctcgccaaacccggagtcatcagtgtgatgggaacctaagcgtgggtggccaacatcatatacctcttgaagaagaaggagtcagccatcgccaacttgtctctgtagaagctccgggtgtagattcccttgcactgtatcatttcatgctttgatttacactcgaactcgggagggaacatcctgctgcatgacctatcagtatggtgctaatgtgtctgtggaccctcgctctctgtctccaggcagttctctcgaatactttgaatgttgtgtaacagttagccactgctggtgtttatgtgaacattcctatcaatccaaattccctctggagtttcatgttatgcctgttgcaggcaaatgtaaagtctagaaaataatgcaaatgtcacggctactctatatacttttgcttggttcattttttttcccttttagttaagcatgactttagatgggaagcctgtgtatcgtggagaaacaagagaccaactttttcattccctgcccccaatttcccagactagatttcaagctaattttctttttctgaagcctctaacaaatgatctagttcagaaggaagcaaaatcccttaatctatgtgcaccgttgggaccaatgccttaattaaagaatttaaaaaagttgtaatagagaatatttttggcattcctctaatgttgtgtgttttttttttgtgtgtgctggagggaggggatttaattttaattttaaaatgtttaggaaatttatacaaagaaactttttaataaagtatattgaaagttt

SEQ ID NO: 168 - DNA, 2259 bp, Homo sapiens
  source 1..2259: mol_type = other DNA
cgataaagcccccgccgccgcggcagccagcttgcgctgtggggctgcccgggctgcgcggcgtctgcaggcgccaccgctgcctctttccggctgtgaccctcctcgccgccgccgcttggctgcgtcctccgactccccgcgccgccgagaccaggctcccgctccggttgcggccgcaccgccctccgcggccgccccctggggatccagcgagcgcggtcgtccttggtggaaggaaccatgaactggcatctccccctcttcctcttggcctctgtgacgctgccttccatctgctcccacttcaatcctctgtctctcgaggaactaggctccaacacggggatccaggttttcaatcagattgtgaagtcgaggcctcatgacaacatcgtgatctctccccatgggattgcgtcggtcctggggatgcttcagctgggggcggacggcaggaccaagaagcagctcgccatggtgatgagatacggcgtaaatggagttggtaaaatattaaagaagatcaacaaggccatcgtctccaagaagaataaagacattgtgacagtggctaacgccgtgtttgttaagaatgcctctgaaattgaagtgccttttgttacaaggaacaaagatgtgttccagtgtgaggtccggaatgtgaactttgaggatccagcctctgcctgtgattccatcaatgcatgggttaaaaatgaaaccagggatatgattgacaatctgctgtccccagatcttattgatggtgtgctcaccagactggtcctcgtcaacgcagtgtatttcaagggtctgtggaaatcacggttccaacccgagaacacaaagaaacgcactttcgtggcagccgacgggaaatcctatcaagtgccaatgctggcccagctctccgtgttccggtgtgggtcgacaagtgcccccaatgatttatggtacaacttcattgaactgccctaccacggggaaagcatcagcatgctgattgcactgccgactgagagctccactccgctgtctgccatcatcccacacatcagcaccaagaccatagacagctggatgagcatcatggtgcccaagagggtgcaggtgatcctgcccaagttcacagctgtagcacaaacagatttgaaggagccgctgaaagttcttggcattactgacatgtttgattcatcaaaggcaaattttgcaaaaataacaacagggtcagaaaacctccatgtttctcatatcttgcaaaaagcaaaaattgaagtcagtgaagatggaaccaaagcttcagcagcaacaactgcaattctcattgcaagatcatcgcctccctggtttatagtagacagaccttttctgtttttcatccgacataatcctacaggtgctgtgttattcatggggcagataaacaaaccctgaagagtatacaaaagaaaccatgcaaagcaacgactactttgctacgaagaaagactcctttcctgcatctttcatagttctgttaaatatttttgtacatcgcttctttttcaaaactagttcttaggaacagactcgatgcaagtgtttctgttctgggaggtattggagggaaaaaacaagcaggatggctggaacactgtactgaggaatgaatagaaaggcttccagatgtctaaaagattctttaaactactgaactgttacctaggttaacaaccctgttgagtatttgctgtttgtccagttcaggaatttttgttttgttttgtctatatgtgcggcttttcagaagaaatttaatcagtgtgacagaaaaaaaaatgttttatggtagcttttactttttatgaaaaaaaaattatttgccttttaaattcttttcccccatccccctccaaagtcttgatagcaagcgttattttgggggtagaaacggtgaaatctctagcctctttgtgtttttgttgttgttgttgttgttgttttatataatgcatgtattcactaaaataaaatttaaaaaactcctgtcttgctagacaaggttgctgttgtgcagtgtgcctgtcactactggtctgtactccttggatttgcatttttgtattttgtacaaagtaaaaataaactgttatgagtagtaaaaataaagctatttctctgctatttgaaaatacaatagaagaaactgagccttttagacattcgtcagcctcttctaataaacctttgtactatgtaaacatcaggaaattc

SEQ ID NO: 169 - DNA, 8500 bp, Homo sapiens
  source 1..8500: mol_type = other DNA
ggccacagcctgcctactgtcacccgcctctcccgcgcgcagatacacgcccccgcctccgtgggcacaaaggcagcgctgctggggaactcgggggaacgcgcacgtgggaaccgccgcagctccacactccaggtacttcttccaaggacctaggtctctcgcccatcggaaagaaaataattctttcaagaagatcagggacaactgatttgaagtctactctgtgcttctaaatccccaattctgctgaaagtgagataccctagagccctagagccccagcagcacccagccaaacccacctccaccatgggggccatgactcagctgttggcaggtgtctttcttgctttccttgccctcgctaccgaaggtggggtcctcaagaaagtcatccggcacaagcgacagagtggggtgaacgccaccctgccagaagagaaccagccagtggtgtttaaccacgtttacaacatcaagctgccagtgggatcccagtgttcggtggatctggagtcagccagtggggagaaagacctggcaccgccttcagagcccagcgaaagctttcaggagcacacagtggatggggaaaaccagattgtcttcacacatcgcatcaacatcccccgccgggcctgtggctgtgccgcagcccctgatgttaaggagctgctgagcagactggaggagctggagaacctggtgtcttccctgagggagcaatgtactgcaggagcaggctgctgtctccagcctgccacaggccgcttggacaccaggcccttctgtagcggtcggggcaacttcagcactgaaggatgtggctgtgtctgcgaacctggctggaaaggccccaactgctctgagcccgaatgtccaggcaactgtcaccttcgaggccggtgcattgatgggcagtgcatctgtgacgacggcttcacgggcgaggactgcagccagctggcttgccccagcgactgcaatgaccagggcaagtgcgtaaatggagtctgcatctgtttcgaaggctacgccggggctgactgcagccgtgaaatctgcccagtgccctgcagtgaggagcacggcacatgtgtagatggcttgtgtgtgtgccacgatggctttgcaggcgatgactgcaacaagcctctgtgtctcaacaattgctacaaccgtggacgatgcgtggagaatgagtgcgtgtgtgatgagggtttcacgggcgaagactgcagtgagctcatctgccccaatgactgcttcgaccggggccgctgcatcaatggcacctgctactgcgaagaaggcttcacaggtgaagactgcgggaaacccacctgcccacatgcctgccacacccagggccggtgtgaggaggggcagtgtgtatgtgatgagggctttgccggtgtggactgcagcgagaagaggtgtcctgctgactgtcacaatcgtggccgctgtgtagacgggcggtgtgagtgtgatgatggtttcactggagctgactgtggggagctcaagtgtcccaatggctgcagtggccatggccgctgtgtcaatgggcagtgtgtgtgtgatgagggctatactggggaggactgcagccagctacggtgccccaatgactgtcacagtcggggccgctgtgtcgagggcaaatgtgtatgtgagcaaggcttcaagggctatgactgcagtgacatgagctgccctaatgactgtcaccagcacggccgctgtgtgaatggcatgtgtgtttgtgatgacggctacacaggggaagactgccgggatcgccaatgccccagggactgcagcaacaggggcctctgtgtggacggacagtgcgtctgtgaggacggcttcaccggccctgactgtgcagaactctcctgtccaaatgactgccatggccagggtcgctgtgtgaatgggcagtgcgtgtgccatgaaggatttatgggcaaagactgcaaggagcaaagatgtcccagtgactgtcatggccagggccgctgcgtggacggccagtgcatctgccacgagggcttcacaggcctggactgtggccagcactcctgccccagtgactgcaacaacttaggacaatgcgtctcgggccgctgcatctgcaacgagggctacagcggagaagactgctcagaggtgtctcctcccaaagacctcgttgtgacagaagtgacggaagagacggtcaacctggcctgggacaatgagatgcgggtcacagagtaccttgtcgtgtacacgcccacccacgagggtggtctggaaatgcagttccgtgtgcctggggaccagacgtccaccatcatccaggagctggagcctggtgtggagtactttatccgtgtatttgccatcctggagaacaagaagagcattcctgtcagcgccagggtggccacgtacttacctgcacctgaaggcctgaaattcaagtccatcaaggagacatctgtggaagtggagtgggatcctctagacattgcttttgaaacctgggagatcatcttccggaatatgaataaagaagatgagggagagatcaccaaaagcctgaggaggccagagacctcttaccggcaaactggtctagctcctgggcaagagtatgagatatctctgcacatagtgaaaaacaatacccggggccctggcctgaagagggtgaccaccacacgcttggatgcccccagccagatcgaggtgaaagatgtcacagacaccactgccttgatcacctggttcaagcccctggctgagatcgatggcattgagctgacctacggcatcaaagacgtgccaggagaccgtaccaccatcgatctcacagaggacgagaaccagtactccatcgggaacctgaagcctgacactgagtacgaggtgtccctcatctcccgcagaggtgacatgtcaagcaacccagccaaagagaccttcacaacaggcctcgatgctcccaggaatcttcgacgtgtttcccagacagataacagcatcaccctggaatggaggaatggcaaggcagctattgacagttacagaattaagtatgcccccatctctggaggggaccacgctgaggttgatgttccaaagagccaacaagccacaaccaaaaccacactcacaggtctgaggccgggaactgaatatgggattggagtttctgctgtgaaggaagacaaggagagcaatccagcgaccatcaacgcagccacagagttggacacgcccaaggaccttcaggtttctgaaactgcagagaccagcctgaccctgctctggaagacaccgttggccaaatttgaccgctaccgcctcaattacagtctccccacaggccagtgggtgggagtgcagcttccaagaaacaccacttcctatgtcctgagaggcctggaaccaggacaggagtacaatgtcctcctgacagccgagaaaggcagacacaagagcaagcccgcacgtgtgaaggcatccactgaacaagcccctgagctggaaaacctcaccgtgactgaggttggctgggatggcctcagactcaactggaccgcagctgaccaggcctatgagcactttatcattcaggtgcaggaggccaacaaggtggaggcagctcggaacctcaccgtgcctggcagccttcgggctgtggacataccgggcctcaaggctgctacgccttatacagtctccatctatggggtgatccagggctatagaacaccagtgctctctgctgaggcctccacaggggaaactcccaatttgggagaggtcgtggtggccgaggtgggctgggatgccctcaaactcaactggactgctccagaaggggcctatgagtactttttcattcaggtgcaggaggctgacacagtagaggcagcccagaacctcaccgtcccaggaggactgaggtccacagacctgcctgggctcaaagcagccactcattataccatcaccatccgcggggtcactcaggacttcagcacaacccctctctctgttgaagtcttgacagaggaggttccagatatgggaaacctcacagtgaccgaggttagctgggatgctctcagactgaactggaccacgccagatggaacctatgaccagtttactattcaggtccaggaggctgaccaggtggaagaggctcacaatctcacggttcctggcagcctgcgttccatggaaatcccaggcctcagggctggcactccttacacagtcaccctgcacggcgaggtcaggggccacagcactcgaccccttgctgtagaggtcgtcacagaggatctcccacagctgggagatttagccgtgtctgaggttggctgggatggcctcagactcaactggaccgcagctgacaatgcctatgagcactttgtcattcaggtgcaggaggtcaacaaagtggaggcagcccagaacctcacgttgcctggcagcctcagggctgtggacatcccgggcctcgaggctgccacgccttatagagtctccatctatggggtgatccggggctatagaacaccagtactctctgctgaggcctccacagccaaagaacctgaaattggaaacttaaatgtttctgacataactcccgagagcttcaatctctcctggatggctaccgatgggatcttcgagacctttaccattgaaattattgattccaataggttgctggagactgtggaatataatatctctggtgctgaacgaactgcccatatctcagggctaccccctagtactgattttattgtctacctctctggacttgctcccagcatccggaccaaaaccatcagtgccacagccacgacagaggccctgccccttctggaaaacctaaccatttccgacattaatccctacgggttcacagtttcctggatggcatcggagaatgcctttgacagctttctagtaacggtggtggattctgggaagctgctggacccccaggaattcacactttcaggaacccagaggaagctggagcttagaggcctcataactggcattggctatgaggttatggtctctggcttcacccaagggcatcaaaccaagcccttgagggctgagattgttacagaagccgaaccggaagttgacaaccttctggtttcagatgccaccccagacggtttccgtctgtcctggacagctgatgaaggggtcttcgacaattttgttctcaaaatcagagataccaaaaagcagtctgagccactggaaataaccctacttgcccccgaacgtaccagggacataacaggtctcagagaggctactgaatacgaaattgaactctatggaataagcaaaggaaggcgatcccagacagtcagtgctatagcaacaacagccatgggctccccaaaggaagtcattttctcagacatcactgaaaattcggctactgtcagctggagggcacccacagcccaagtggagagcttccggattacctatgtgcccattacaggaggtacaccctccatggtaactgtggacggaaccaagactcagaccaggctggtgaaactcatacctggcgtggagtaccttgtcagcatcatcgccatgaagggctttgaggaaagtgaacctgtctcagggtcattcaccacagctctggatggcccatctggcctggtgacagccaacatcactgactcagaagccttggccaggtggcagccagccattgccactgtggacagttatgtcatctcctacacaggcgagaaagtgccagaaattacacgcacggtgtccgggaacacagtggagtatgctctgaccgacctcgagcctgccacggaatacacactgagaatctttgcagagaaagggccccagaagagctcaaccatcactgccaagttcacaacagacctcgattctccaagagacttgactgctactgaggttcagtcggaaactgccctccttacctggcgacccccccgggcatcagtcaccggttacctgctggtctatgaatcagtggatggcacagtcaaggaagtcattgtgggtccagataccacctcctacagcctggcagacctgagcccatccacccactacacagccaagatccaggcactcaatgggcccctgaggagcaatatgatccagaccatcttcaccacaattggactcctgtaccccttccccaaggactgctcccaagcaatgctgaatggagacacgacctctggcctctacaccatttatctgaatggtgataaggctgaggcgctggaagtcttctgtgacatgacctctgatgggggtggatggattgtgttcctgagacgcaaaaacggacgcgagaacttctaccaaaactggaaggcatatgctgctggatttggggaccgcagagaagaattctggcttgggctggacaacctgaacaaaatcacagcccaggggcagtacgagctccgggtggacctgcgggaccatggggagacagcctttgctgtctatgacaagttcagcgtgggagatgccaagactcgctacaagctgaaggtggaggggtacagtgggacagcaggtgactccatggcctaccacaatggcagatccttctccacctttgacaaggacacagattcagccatcaccaactgtgctctgtcctacaaaggggctttctggtacaggaactgtcaccgtgtcaacctgatggggagatatggggacaataaccacagtcagggcgttaactggttccactggaagggccacgaacactcaatccagtttgctgagatgaagctgagaccaagcaacttcagaaatcttgaaggcaggcgcaaacgggcataaattccagggaccactgggtgagagaggaataaggcccagagcgaggaaaggattttaccaaagcatcaatacaaccagcccaaccatcggtccacacctgggcatttggtgagagtcaaagctgaccatggatccctggggccaacggcaacagcatgggcctcacctcctctgtgatttctttctttgcaccaaagacatcagtctccaacatgtttctgttttgttgtttgattcagcaaaaatctcccagtgacaacatcgcaatagttttttacttctcttaggtggctctgggaatgggagaggggtaggatgtacaggggtagtttgttttagaaccagccgtattttacatgaagctgtataattaattgtcattatttttgttagcaaagattaaatgtgtcattggaagccatccctttttttacatttcatacaacagaaaccagaaaagcaatactgtttccattttaaggatatgattaatattattaatataataatgatgatgatgatgatgaaaactaaggatttttcaagagatctttctttccaaaacatttctggacagtacctgattgtattttttttttaaataaaagcacaagtacttttgagtttgttattttgctttgaattgttgagtctgaatttcaccaaagccaatcatttgaacaaagcggggaatgttgggataggaaaggtaagtagggatagtggtcaagtgggaggggtggaaaggagactaaagactgggagagagggaagcactttttttaaataaagttgaacacacttgggaaaagcttacaggccaggcctgtaatcccaacactttgggaggccaaggtgggaggatagcttaaccccaggagtttgagaccagcctgagcaacatagtgagaacttgtctctacagaaaaaaaaaaaaaaaaaaatttaattaggcaagcgtggtagtgcgcacctgtcgtcccagctactcaggaggctgaggtaggaaaatcactggagcccaggagttagaggttacagtgagctatgatcacactactgcactccagcctgggcaacagagggagaccctgtctctaaataaaaaaagaaaagaaaaaaaaagcttacaacttgagattcagcatcttgctcagtatttccaagactaatagattatggtttaaaagatgcttttatactcattttctaatgcaactcctagaaactctatgatatagttgaggtaagtattgttaccacacatgggctaagatccccagaggcagactgcctgagttcaattcttggctccaccattcccaagttccctaacctctctatgcctcagtttcctcttctgtaaagtagggacactcatacttctcatttcagaacatttttgtgaagaataaattatgttatccatttgaggcccttagaatggtacccggtgtatattaagtgctagtacatgttagctatcatcattatcactttatatgagatggactggggttcatagaaacccaatgacttgattgtggctactactcaataaataatagaatttggatttaaa

SEQ ID NO: 170 - DNA, 2452 bp, Homo sapiens
  source 1..2452: mol_type = other DNA
agaaagcgagcagccacccagctccccgccaccgccatggtccccgacaccgcctgcgttcttctgctcaccctggctgccctcggcgcgtccggacagggccagagcccgttgggctcagacctgggcccgcagatgcttcgggaactgcaggaaaccaacgcggcgctgcaggacgtgcgggagctgctgcggcagcaggtcagggagatcacgttcctgaaaaacacggtgatggagtgtgacgcgtgcgggatgcagcagtcagtacgcaccggcctacccagcgtgcggcccctgctccactgcgcgcccggcttctgcttccccggcgtggcctgcatccagacggagagcggcgcgcgctgcggcccctgccccgcgggcttcacgggcaacggctcgcactgcaccgacgtcaacgagtgcaacgcccacccctgcttcccccgagtccgctgtatcaacaccagcccggggttccgctgcgaggcttgcccgccggggtacagcggccccacccaccagggcgtggggctggctttcgccaaggccaacaagcaggtttgcacggacatcaacgagtgtgagaccgggcaacataactgcgtccccaactccgtgtgcatcaacacccggggctccttccagtgcggcccgtgccagcccggcttcgtgggcgaccaggcgtccggctgccagcggcgcgcacagcgcttctgccccgacggctcgcccagcgagtgccacgagcatgcagactgcgtcctagagcgcgatggctcgcggtcgtgcgtgtgtgccgttggctgggccggcaacgggatcctctgtggtcgcgacactgacctagacggcttcccggacgagaagctgcgctgcccggagcgccagtgccgtaaggacaactgcgtgactgtgcccaactcagggcaggaggatgtggaccgcgatggcatcggagacgcctgcgatccggatgccgacggggacggggtccccaatgaaaaggacaactgcccgctggtgcggaacccagaccagcgcaacacggacgaggacaagtggggcgatgcgtgcgacaactgccggtcccagaagaacgacgaccaaaaggacacagaccaggacggccggggcgatgcgtgcgacgacgacatcgacggcgaccggatccgcaaccaggccgacaactgccctagggtacccaactcagaccagaaggacagtgatggcgatggtataggggatgcctgtgacaactgtccccagaagagcaacccggatcaggcggatgtggaccacgactttgtgggagatgcttgtgacagcgatcaagaccaggatggagacggacatcaggactctcgggacaactgtcccacggtgcctaacagtgcccaggaggactcagaccacgatggccagggtgatgcctgcgacgacgacgacgacaatgacggagtccctgacagtcgggacaactgccgcctggtgcctaaccccggccaggaggacgcggacagggacggcgtgggcgacgtgtgccaggacgactttgatgcagacaaggtggtagacaagatcgacgtgtgtccggagaacgctgaagtcacgctcaccgacttcagggccttccagacagtcgtgctggacccggagggtgacgcgcagattgaccccaactgggtggtgctcaaccagggaagggagatcgtgcagacaatgaacagcgacccaggcctggctgtgggttacactgccttcaatggcgtggacttcgagggcacgttccatgtgaacacggtcacggatgacgactatgcgggcttcatctttggctaccaggacagctccagcttctacgtggtcatgtggaagcagatggagcaaacgtattggcaggcgaaccccttccgtgctgtggccgagcctggcatccaactcaaggctgtgaagtcttccacaggccccggggaacagctgcggaacgctctgtggcatacaggagacacagagtcccaggtgcggctgctgtggaaggacccgcgaaacgtgggttggaaggacaagaagtcctatcgttggttcctgcagcaccggccccaagtgggctacatcagggtgcgattctatgagggccctgagctggtggccgacagcaacgtggtcttggacacaaccatgcggggtggccgcctgggggtcttctgcttctcccaggagaacatcatctgggccaacctgcgttaccgctgcaatgacaccatcccagaggactatgagacccatcagctgcggcaagcctagggaccagggtgaggacccgccggatgacagccaccctcaccgcggctggatgggggctctgcacccagccccaaggggtggccgtcctgagggggaagtgagaagggctcagagaggacaaaataaagtgtgtgtgcaggga

SEQ ID NO: 171 - DNA, 7789 bp, Homo sapiens
  source 1..7789: mol_type = other DNA
agccgctgcgcccgagctggcctgcgagttcagggctcctgtcgctctccaggagcaacctctactccggacgcacaggcattccccgcgcccctccagccctcgccgccctcgccaccgctcccggccgccgcgctccggtacacacaggatccctgctgggcaccaacagctccaccatggggctggcctggggactaggcgtcctgttcctgatgcatgtgtgtggcaccaaccgcattccagagtctggcggagacaacagcgtgtttgacatctttgaactcaccggggccgcccgcaaggggtctgggcgccgactggtgaagggccccgacccttccagcccagctttccgcatcgaggatgccaacctgatcccccctgtgcctgatgacaagttccaagacctggtggatgctgtgcgggcagaaaagggtttcctccttctggcatccctgaggcagatgaagaagacccggggcacgctgctggccctggagcggaaagaccactctggccaggtcttcagcgtggtgtccaatggcaaggcgggcaccctggacctcagcctgaccgtccaaggaaagcagcacgtggtgtctgtggaagaagctctcctggcaaccggccagtggaagagcatcaccctgtttgtgcaggaagacagggcccagctgtacatcgactgtgaaaagatggagaatgctgagttggacgtccccatccaaagcgtcttcaccagagacctggccagcatcgccagactccgcatcgcaaaggggggcgtcaatgacaatttccagggggtgctgcagaatgtgaggtttgtctttggaaccacaccagaagacatcctcaggaacaaaggctgctccagctctaccagtgtcctcctcacccttgacaacaacgtggtgaatggttccagccctgccatccgcactaactacattggccacaagacaaaggacttgcaagccatctgcggcatctcctgtgatgagctgtccagcatggtcctggaactcaggggcctgcgcaccattgtgaccacgctgcaggacagcatccgcaaagtgactgaagagaacaaagagttggccaatgagctgaggcggcctcccctatgctatcacaacggagttcagtacagaaataacgaggaatggactgttgatagctgcactgagtgtcactgtcagaactcagttaccatctgcaaaaaggtgtcctgccccatcatgccctgctccaatgccacagttcctgatggagaatgctgtcctcgctgttggcccagcgactctgcggacgatggctggtctccatggtccgagtggacctcctgttctacgagctgtggcaatggaattcagcagcgcggccgctcctgcgatagcctcaacaaccgatgtgagggctcctcggtccagacacggacctgccacattcaggagtgtgacaagagatttaaacaggatggtggctggagccactggtccccgtggtcatcttgttctgtgacatgtggtgatggtgtgatcacaaggatccggctctgcaactctcccagcccccagatgaacgggaaaccctgtgaaggcgaagcgcgggagaccaaagcctgcaagaaagacgcctgccccatcaatggaggctggggtccttggtcaccatgggacatctgttctgtcacctgtggaggaggggtacagaaacgtagtcgtctctgcaacaaccccacaccccagtttggaggcaaggactgcgttggtgatgtaacagaaaaccagatctgcaacaagcaggactgtccaattgatggatgcctgtccaatccctgctttgccggcgtgaagtgtactagctaccctgatggcagctggaaatgtggtgcttgtccccctggttacagtggaaatggcatccagtgcacagatgttgatgagtgcaaagaagtgcctgatgcctgcttcaaccacaatggagagcaccggtgtgagaacacggaccccggctacaactgcctgccctgccccccacgcttcaccggctcacagcccttcggccagggtgtcgaacatgccacggccaacaaacaggtgtgcaagccccgtaacccctgcacggatgggacccacgactgcaacaagaacgccaagtgcaactacctgggccactatagcgaccccatgtaccgctgcgagtgcaagcctggctacgctggcaatggcatcatctgcggggaggacacagacctggatggctggcccaatgagaacctggtgtgcgtggccaatgcgacttaccactgcaaaaaggataattgccccaaccttcccaactcagggcaggaagactatgacaaggatggaattggtgatgcctgtgatgatgacgatgacaatgataaaattccagatgacagggacaactgtccattccattacaacccagctcagtatgactatgacagagatgatgtgggagaccgctgtgacaactgtccctacaaccacaacccagatcaggcagacacagacaacaatggggaaggagacgcctgtgctgcagacattgatggagacggtatcctcaatgaacgggacaactgccagtacgtctacaatgtggaccagagagacactgatatggatggggttggagatcagtgtgacaattgccccttggaacacaatccggatcagctggactctgactcagaccgcattggagatacctgtgacaacaatcaggatattgatgaagatggccaccagaacaatctggacaactgtccctatgtgcccaatgccaaccaggctgaccatgacaaagatggcaagggagatgcctgtgaccacgatgatgacaacgatggcattcctgatgacaaggacaactgcagactcgtgcccaatcccgaccagaaggactctgacggcgatggtcgaggtgatgcctgcaaagatgattttgaccatgacagtgtgccagacatcgatgacatctgtcctgagaatgttgacatcagtgagaccgatttccgccgattccagatgattcctctggaccccaaagggacatcccaaaatgaccctaactgggttgtacgccatcagggtaaagaactcgtccagactgtcaactgtgatcctggactcgctgtaggttatgatgagtttaatgctgtggacttcagtggcaccttcttcatcaacaccgaaagggacgatgactatgctggatttgtctttggctaccagtccagcagccgcttttatgttgtgatgtggaagcaagtcacccagtcctactgggacaccaaccccacgagggctcagggatactcgggcctttctgtgaaagttgtaaactccaccacagggcctggcgagcacctgcggaacgccctgtggcacacaggaaacacccctggccaggtgcgcaccctgtggcatgaccctcgtcacataggctggaaagatttcaccgcctacagatggcgtctcagccacaggccaaagacgggtttcattagagtggtgatgtatgaagggaagaaaatcatggctgactcaggacccatctatgataaaacctatgctggtggtagactagggttgtttgtcttctctcaagaaatggtgttcttctctgacctgaaatacgaatgtagagatccctaatcatcaaattgttgattgaaagactgatcataaaccaatgctggtattgcaccttctggaactatgggcttgagaaaacccccaggatcacttctccttggcttccttcttttctgtgcttgcatcagtgtggactcctagaacgtgcgacctgcctcaagaaaatgcagttttcaaaaacagactcagcattcagcctccaatgaataagacatcttccaagcatataaacaattgctttggtttccttttgaaaaagcatctacttgcttcagttgggaaggtgcccattccactctgcctttgtcacagagcagggtgctattgtgaggccatctctgagcagtggactcaaaagcattttcaggcatgtcagagaagggaggactcactagaattagcaaacaaaaccaccctgacatcctccttcaggaacacggggagcagaggccaaagcactaaggggagggcgcatacccgagacgattgtatgaagaaaatatggaggaactgttacatgttcggtactaagtcattttcaggggattgaaagactattgctggatttcatgatgctgactggcgttagctgattaacccatgtaaataggcacttaaatagaagcaggaaagggagacaaagactggcttctggacttcctccctgatccccacccttactcatcacctgcagtggccagaattagggaatcagaatcaaaccagtgtaaggcagtgctggctgccattgcctggtcacattgaaattggtggcttcattctagatgtagcttgtgcagatgtagcaggaaaataggaaaacctaccatctcagtgagcaccagctgcctcccaaaggaggggcagccgtgcttatatttttatggttacaatggcacaaaattattatcaacctaactaaaacattccttttctcttttttcctgaattatcatggagttttctaattctctcttttggaatgtagattttttttaaatgctttacgatgtaaaatatttattttttacttattctggaagatctggctgaaggattattcatggaacaggaagaagcgtaaagactatccatgtcatctttgttgagagtcttcgtgactgtaagattgtaaatacagattatttattaactctgttctgcctggaaatttaggcttcatacggaaagtgtttgagagcaagtagttgacatttatcagcaaatctcttgcaagaacagcacaaggaaaatcagtctaataagctgctctgccccttgtgctcagagtggatgttatgggattctttttttctctgttttatcttttcaagtggaattagttggttatccatttgcaaatgttttaaattgcaaagaaagccatgaggtcttcaatactgttttaccccatcccttgtgcatatttccagggagaaggaaagcatatacacttttttctttcatttttccaaaagagaaaaaaatgacaaaaggtgaaacttacatacaaatattacctcatttgttgtgtgactgagtaaagaatttttggatcaagcggaaagagtttaagtgtctaacaaacttaaagctactgtagtacctaaaaagtcagtgttgtacatagcataaaaactctgcagagaagtattcccaataaggaaatagcattgaaatgttaaatacaatttctgaaagttatgttttttttctatcatctggtataccattgctttatttttataaattattttctcattgccattggaatagatatctcagattgtgtagatatgctatttaaataatttatcaggaaatactgcctgtagagttagtatttctatttttatataatgtttgcacactgaattgaagaattgttggttttttcttttttttgttttgtttttttttttttttttttttgcttttgacctcccatttttactatttgccaatacctttttctaggaatgtgcttttttttgtacacatttttatccattttacattctaaagcagtgtaagttgtatattactgtttcttatgtacaaggaacaacaataaatcatatggaaatttatatttatacttactgtatccatgcttatttgttctctactggctttatgtcatgaagtatatgcgtaaataccattcataaatcaatatagcatatacaaaaataaattacagtaagtcatagcaacattcacagtttgtatgtgattgagaaagactgagttgctcaggcctaggcttagaatttgctgcgtttgtggaataaaagaacaaaatgatacattagcctgccatatcaaaaacatataaaagagaaattatccctaagtcaagggcccccataagaataaaatttcttattaaggtcattagatgtcattgaatccttttcaaagtgcagtatgaaaacaaagggaaaaacactgaagcacacgcaactctcacagcgacattttctgacccacgaatgatgccttgggtgggcaacacgattgcatgttgtggagacacttcggaagtaaatgtggatgagggaggagctgtccttgcaatgttgagccaagcattacagatacctcctcttgaagaaggaataataagtttaatcaaaaaagaagactaaaaaatgtaaaatttggaaggaatccataaatgcgtgtgtgtctaaatacaaattatcatgtgaagaaaaggcccaagtgtaccaataagcagaccttgatttttggatgggctaattatgaatgtggaatactgaccagttaatttccagttttaatgaaaacagatcaaagaagaaattttatgagtaggttaaaggtctggctttgaggtctattaaacactagaaaggactggctgggtgagataaaatcttccttgttgattttcactctcattctataaatactcatctttctgagtagccatgatcacatacaaatgtaaattgccaaatcattttatagtaccaaggtgaagaagcaggaactagaaagtgttgataatagctgtggagttaggaaaactgatgtgaaggaaataattctttgaaatggcaaagaattaaataccatcattcattatcagaagagttcaacgtttgaagtgctgggagataattctaattcattcttggatagtgaagcaaaactgattgaaaataccaagataagacagaaaaagtgactggaaagaggagcttttcttccaggcatgttccagtttcaccctaagactgaccttcaaataatcaggttgtactgaaataaaggacttgttaaaaattaaaattatgtcatcgagatgatagcttttttcctcctccaacagtttattgtcatgtgttgtgggagagctcgagtgaagagcaataaactccaggtcttataagaatgtacatacaataaaggtggtgccagcagtttttttttttctaaagagtcacatgtagaaaagcctccagtattaagctcctgaattcattccttaaataaattggctctctctctcttctataatttctttttctttttatttttgagatgaagtcttgctctgtcgcccaggctggagtgcagtgacacaatctcggctcactgcaacctctgcctccccggttcaagcaattctccctcctgcctcagcctcccaagtagctgggactacaagcgcccgccaccaagcctggctaattctgtatttttagtaaagacggggtttcaccttgttccggacaaacactaagccctaaagggaaatccaaaataaaaacatctatttttaataacactttctatctaaatcagggtgactttttaaaaaaaatccggaagctttttgttgaattacgttacagacttagttaccagtccttgttagagttaccttcagttgacatgctgtgaatggtcccacctcttttatggcagaattcattacttaaaataactctattttcttcccccttacctaaataacagaaaggctcactatgtcccaaatatcattggcagaagcaaactataaagtcataagccctttgcagtgcaagtctagaaataatttt

SEQ ID NO: 172 - DNA, 7311 bp, Homo sapiens
  source 1..7311: mol_type = other DNA
actgacggcatgaagcctttaggggcacacagtactctcagcttgttggtggaagcccctcatctgccttcattctgaaggcagggcccggcagaggaaggatcagagggtcgcggccggagggtcccggccggtggggccaactcagagggagaggaaagggctagagacacgaagaacgcaaaccatcaaatttagaagaaaaagccctttgactttttccccctctccctccccaatggctgtgtagcaaacatccctggcgataccttggaaaggacgaagttggtctgcagtcgcaatttcgtgggttgagttcacagttgtgagtgcggggctcggagatggagccgtggtcctctaggtggaaaacgaaacggtggctctgggatttcaccgtaacaaccctcgcattgaccttcctcttccaagctagagaggtcagaggagctgctccagttgatgtactaaaagcactagattttcacaattctccagagggaatatcaaaaacaacgggattttgcacaaacagaaagaattctaaaggctcagatactgcttacagagtttcaaagcaagcacaactcagtgccccaacaaaacagttatttccaggtggaactttcccagaagacttttcaatactatttacagtaaaaccaaaaaaaggaattcagtctttccttttatctatatataatgagcatggtattcagcaaattggtgttgaggttgggagatcacctgtttttctgtttgaagaccacactggaaaacctgccccagaagactatcccctcttcagaactgttaacatcgctgacgggaagtggcatcgggtagcaatcagcgtggagaagaaaactgtgacaatgattgttgattgtaagaagaaaaccacgaaaccacttgatagaagtgagagagcaattgttgataccaatggaatcacggtttttggaacaaggattttggatgaagaagtttttgagggggacattcagcagtttttgatcacaggtgatcccaaggcagcatatgactactgtgagcattatagtccagactgtgactcttcagcacccaaggctgctcaagctcaggaacctcagatagatgagtatgcaccagaggatataatcgaatatgactatgagtatggggaagcagagtataaagaggctgaaagtgtaacagagggacccactgtaactgaggagacaatagcacagacggaggcaaacatcgttgatgattttcaagaatacaactatggaacaatggaaagttaccagacagaagctcctaggcatgtttctgggacaaatgagccaaatccagttgaagaaatatttactgaagaatatctaacgggagaggattatgattcccagaggaaaaattctgaggatacactatatgaaaacaaagaaatagacggcagggattctgatcttctggtagatggagatttaggcgaatatgatttttatgaatataaagaatatgaagataaaccaacaagcccccctaatgaagaatttggtccaggtgtaccagcagaaactgatattacagaaacaagcataaatggccatggtgcatatggagagaaaggacagaaaggagaaccagcagtggttgagcctggtatgcttgtcgaaggaccaccaggaccagcaggacctgcaggtattatgggtcctccaggtctacaaggccccactggaccccctggtgaccctggcgataggggccccccaggacgtcctggcttaccaggggctgatggtctacctggtcctcctggtactatgttgatgttaccgttccgttatggtggtgatggttccaaaggaccaaccatctctgctcaggaagctcaggctcaagctattcttcagcaggctcggattgctctgagaggcccacctggcccaatgggtctaactggaagaccaggtcctgtgggggggcctggttcatctggggccaaaggtgagagtggtgatccaggtcctcagggccctcgaggcgtccagggtccccctggtccaacgggaaaacctggaaaaaggggtcgtccaggtgcagatggaggaagaggaatgccaggagaacctggggcaaagggagatcgagggtttgatggacttccgggtctgccaggtgacaaaggtcacaggggtgaacgaggtcctcaaggtcctccaggtcctcctggtgatgatggaatgaggggagaagatggagaaattggaccaagaggtcttccaggtgaagctggcccacgaggtttgctgggtccaaggggaactccaggagctccagggcagcctggtatggcaggtgtagatggccccccaggaccaaaagggaacatgggtccccaaggggagcctgggcctccaggtcaacaagggaatccaggacctcagggtcttcctggtccacaaggtccaattggtcctcctggtgaaaaaggaccacaaggaaaaccaggacttgctggacttcctggtgctgatgggcctcctggtcatcctgggaaagaaggccagtctggagaaaagggggctctgggtccccctggtccacaaggtcctattggatacccgggcccccggggagtaaagggagcagatggtgtcagaggtctcaagggatctaaaggtgaaaagggtgaagatggttttccaggattcaaaggtgacatgggtctaaaaggtgacagaggagaagttggtcaaattggcccaagaggggaagatggccctgaaggacccaaaggtcgagcaggcccaactggagacccaggtccttcaggtcaagcaggagaaaagggaaaacttggagttccaggattaccaggatatccaggaagacaaggtccaaagggttccactggattccctgggtttccaggtgccaatggagagaaaggtgcacggggagtagctggcaaaccaggccctcggggtcagcgtggtccaacgggtcctcgaggttcaagaggtgcaagaggtcccactgggaaacctgggccaaagggcacttcaggtggcgatggccctcctggccctccaggtgaaagaggtcctcaaggacctcagggtccagttggattccctggaccaaaaggccctcctggaccacctgggaaggatgggctgccaggacaccctgggcaacgtggggagactggatttcaaggcaagaccggccctcctgggccagggggagtggttggaccacagggaccaaccggtgagactggtccaataggggaacgtgggcatcctggccctcctggccctcctggtgagcaaggtcttcctggtgctgcaggaaaagaaggtgcaaagggtgatccaggtcctcaaggtatctcagggaaagatggaccagcaggattacgtggtttcccaggggaaagaggtcttcctggagctcagggtgcacctggactgaaaggaggggaaggtccccagggcccaccaggtccagttggctcaccaggagaacgtgggtcagcaggtacagctggcccaattggtttaccagggcgcccgggacctcagggtcctcctggtccagctggagagaaaggtgctcctggagaaaaaggtccccaagggcctgcagggagagatggagttcaaggtcctgttggtctcccagggccagctggtcctgccggctcccctggggaagacggagacaagggtgaaattggtgagccgggacaaaaaggcagcaagggtgacaagggagaaaatggccctcccggtcccccaggtcttcaaggaccagttggtgcccctggaattgctggaggtgatggtgaaccaggtcctagaggacagcaggggatgtttgggcaaaaaggtgatgagggtgccagaggcttccctggacctcctggtccaataggtcttcagggtctgccaggcccacctggtgaaaaaggtgaaaatggggatgttggtcccatggggccacctggtcctccaggcccaagaggccctcaaggtcccaatggagctgatggaccacaaggacccccagggtctgttggttcagttggtggtgttggagaaaagggtgaacctggagaagcagggaacccagggcctcctggggaagcaggtgtaggcggtcccaaaggagaaagaggagagaaaggggaagctggtccacctggagctgctggacctccaggtgccaaggggccaccaggtgatgatggccctaagggtaacccgggtcctgttggttttcctggagatcctggtcctcctggggaacctggccctgcaggtcaagatggtgttggtggtgacaagggtgaagatggagatcctggtcaaccgggtcctcctggcccatctggtgaggctggcccaccaggtcctcctggaaaacgaggtcctcctggagctgcaggtgcagagggaagacaaggtgaaaaaggtgctaagggggaagcaggtgcagaaggtcctcctggaaaaaccggcccagtcggtcctcagggacctgcaggaaagcctggtccagaaggtcttcggggcatccctggtcctgtgggagaacaaggtctccctggagctgcaggccaagatggaccacctggtcctatgggacctcctggcttacctggtctcaaaggtgaccctggctccaagggtgaaaagggacatcctggtttaattggcctgattggtcctccaggagaacaaggggaaaaaggtgaccgagggctccctggaactcaaggatctccaggagcaaaaggggatgggggaattcctggtcctgctggtcccttaggtccacctggtcctccaggtttaccaggtcctcaaggcccaaagggtaacaaaggctctactggacccgctggccagaaaggtgacagtggtcttccagggcctcctgggtctccaggtccacctggtgaagtcattcagcctttaccaatcttgtcctccaaaaaaacgagaagacatactgaaggcatgcaagcagatgcagatgataatattcttgattactcggatggaatggaagaaatatttggttccctcaattccctgaaacaagacattgagcatatgaaatttccaatgggtactcagaccaatccagcccgaacttgtaaagacctgcaactcagccatcctgacttcccagatggtgaatattggattgatcctaaccaaggttgctcaggagattccttcaaagtttactgtaatttcacatctggtggtgagacttgcatttatccagacaaaaaatctgagggagtaagaatttcatcatggccaaaggagaaaccaggaagttggtttagtgaatttaagaggggaaaactgctttcatacttagatgttgaaggaaattccatcaatatggtgcaaatgacattcctgaaacttctgactgcctctgctcggcaaaatttcacctaccactgtcatcagtcagcagcctggtatgatgtgtcatcaggaagttatgacaaagcacttcgcttcctgggatcaaatgatgaggagatgtcctatgacaataatccttttatcaaaacactgtatgatggttgtgcgtccagaaaaggctatgaaaagactgtcattgaaatcaatacaccaaaaattgatcaagtacctattgttgatgtcatgatcaatgactttggtgatcagaatcagaagttcggatttgaagttggtcctgtttgttttcttggctaagattaagacaaagaacatatcaaatcaacagaaaatataccttggtgccaccaacccattttgtgccacatgcaagttttgaataaggatggtatagaaaacaacgctgcatatacaggtaccatttaggaaataccgatgcctttgtgggggcagaatcacatggcaaaagctttgaaaatcataaagatataagttggtgtggctaagatggaaacagggctgattcttgattcccaattctcaactctccttttcctatttgaatttctttggtgctgtagaaaacaaaaaaagaaaaatatatattcataaaaaatatggtgctcattctcatccatccaggatgtactaaaacagtgtgtttaataaattgtaattattttgtgtacagttctatactgttatctgtgtccatttccaaaacttgcacgtgtccctgaattccatctgactctaattttatgagaattgcagaactctgatggcaataaatatatgtattatgaaaaaataaagttgtaatttctgatgactctaagtccctttctttggttaataataaaatgcctttgtatatattgatgttgaagagttcaattatttgatgtcgccaacaaaattctcagagggcaaaaatctggaagacttttggaagcacactctgatcaactcttctctgccgacagtcattttgctgaatttcagccaaaaatattatgcattttgatgctttattcaaggctatacctcaaactttttcttctcagaatccaggatttcacaggatacttgtatatatggaaaacaagcaagtttatatttttggacagggaaatgtgtgtaagaaagtatattaacaaatcaatgcctccgtcaagcaaacaatcatatgtatactttttttctacgttatctcatctccttgttttcagtgtgcttcaataatgcaggttaatattaaagatggaaattaagcaattatttatgaatttgtgcaatgttagattttcttatcaatcaagttcttgaatttgattctaagttgcatattataacagtctcgaaaattattttacttgcccaacaaatattacttttttcctttcaagataattttataaatcatttgacctacctaattgctaaatgaataacatatggtggactgttattaagagtatttgttttaagtcattcaggaaaatctaaacttttttttccactaaggtatttactttaaggtagcttgaaatagcaatacaatttaaaaattaaaaactgaattttgtatctattttaagtaatatatgtaagacttgaaaataaatgttttatttcttatataaagtgttaaattaattgataccagatttcactggaacagtttcaactgataatttatgacaaaagaacatacctgtaatattgaaattaaaaagtgaaatttgtcataaagaatttcttttatttttgaaatcgagtttgtaaatgtccttttaagaagggagatatgaatccaataaataaactcaagtcttggcta

SEQ ID NO: 173 - DNA, 5914 bp, Homo sapiens
  source 1..5914: mol_type = other DNA
gcagacgggagtttctcctcggggtcggagcaggaggcacgcggagtgtgaggccacgcatgagcggacgctaaccccctccccagccacaaagagtctacatgtctagggtctagacatgttcagctttgtggacctccggctcctgctcctcttagcggccaccgccctcctgacgcacggccaagaggaaggccaagtcgagggccaagacgaagacatcccaccaatcacctgcgtacagaacggcctcaggtaccatgaccgagacgtgtggaaacccgagccctgccggatctgcgtctgcgacaacggcaaggtgttgtgcgatgacgtgatctgtgacgagaccaagaactgccccggcgccgaagtccccgagggcgagtgctgtcccgtctgccccgacggctcagagtcacccaccgaccaagaaaccaccggcgtcgagggacccaagggagacactggcccccgaggcccaaggggacccgcaggcccccctggccgagatggcatccctggacagcctggacttcccggaccccccggaccccccggacctcccggaccccctggcctcggaggaaactttgctccccagctgtcttatggctatgatgagaaatcaaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggtgctcgtggaaatgatggtgctactggtgctgccgggccccctggtcccaccggccccgctggtcctcctggcttccctggtgctgttggtgctaagggtgaagctggtccccaagggccccgaggctctgaaggtccccagggtgtgcgtggtgagcctggcccccctggccctgctggtgctgctggccctgctggaaaccctggtgctgatggacagcctggtgctaaaggtgccaatggtgctcctggtattgctggtgctcctggcttccctggtgcccgaggcccctctggaccccagggccccggcggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccctgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggtccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgactggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgctaaaggcgatgctggtccccctggccctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggtcctgctggtgctcctggtactcccgggcctcaaggtattgctggacagcgtggtgtggtcggcctgcctggtcagagaggagagagaggcttccctggtcttcctggcccctctggtgaacctggcaaacaaggtccctctggagcaagtggtgaacgtggtccccctggtcccatgggcccccctggattggctggaccccctggtgaatctggacgtgagggggctcctggtgccgaaggttcccctggacgagacggttctcctggcgccaagggtgaccgtggtgagaccggccccgctggaccccctggtgctcctggtgctcctggtgcccctggccccgttggccctgctggcaagagtggtgatcgtggtgagactggtcctgctggtcccgccggtcctgtcggccctgttggcgcccgtggccccgccggaccccaaggcccccgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggcagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggccatccagctgaccttcctgcgcctgatgtccaccgaggcctcccagaacatcacctaccactgcaagaacagcgtggcctacatggaccagcagactggcaacctcaagaaggccctgctcctccagggctccaacgagatcgagatccgcgccgagggcaacagccgcttcacctacagcgtcactgtcgatggctgcacgagtcacaccggagcctggggcaagacagtgattgaatacaaaaccaccaagacctcccgcctgcccatcatcgatgtggcccccttggacgttggtgccccagaccaggaattcggcttcgacgttggccctgtctgcttcctgtaaactccctccatcccaacctggctccctcccacccaaccaactttccccccaacccggaaacagacaagcaacccaaactgaaccccctcaaaagccaaaaaatgggagacaatttcacatggactttggaaaatatttttttcctttgcattcatctctcaaacttagtttttatctttgaccaaccgaacatgaccaaaaaccaaaagtgcattcaaccttaccaaaaaaaaaaaaaaaaaaagaataaataaataactttttaaaaaaggaagcttggtccacttgcttgaagacccatgcgggggtaagtccctttctgcccgttgggcttatgaaaccccaatgctgccctttctgctcctttctccacaccccccttggggcctcccctccactccttcccaaatctgtctccccagaagacacaggaaacaatgtattgtctgcccagcaatcaaaggcaatgctcaaacacccaagtggcccccaccctcagcccgctcctgcccgcccagcacccccaggccctgggggacctggggttctcagactgccaaagaagccttgccatctggcgctcccatggctcttgcaacatctccccttcgtttttgagggggtcatgccgggggagccaccagcccctcactgggttcggaggagagtcaggaagggccacgacaaagcagaaacatcggatttggggaacgcgtgtcaatcccttgtgccgcagggctgggcgggagagactgttctgttccttgtgtaactgtgttgctgaaagactacctcgttcttgtcttgatgtgtcaccggggcaactgcctgggggcggggatgggggcagggtggaagcggctccccattttataccaaaggtgctacatctatgtgatgggtggggtggggagggaatcactggtgctatagaaattgagatgcccccccaggccagcaaatgttcctttttgttcaaagtctatttttattccttgatatttttctttttttttttttttttttgtggatggggacttgtgaatttttctaaaggtgctatttaacatgggaggagagcgtgtgcggctccagcccagcccgctgctcactttccaccctctctccacctgcctctggcttctcaggcctctgctctccgacctctctcctctgaaaccctcctccacagctgcagcccatcctcccggctccctcctagtctgtcctgcgtcctctgtccccgggtttcagagacaacttcccaaagcacaaagcagtttttccccctaggggtgggaggaagcaaaagactctgtacctattttgtatgtgtataataatttgagatgtttttaattattttgattgctggaataaagcatgtggaaatgacccaaa

SEQ ID NO: 174 - DNA, 6829 bp, Homo sapiens
  source 1..6829: mol_type = other DNA
gcagactgtgctggagctggtgctgaaaaagggggtttgcagaggctgccctggggctggtgctgaaagaagagcccacagctgacttcatggtgctacaataacctcagaatctacttttcactctcaggagaacccacatgtctaatatttagacatgatggcaaactgggcggaagcaagacctctcctcattcttattgttttattagggcaatttgtctcaataaaagcccaggaagaagacgaggatgaaggatatggtgaagaaatagcctgcactcagaatggccagatgtacttaaacagggacatttggaaacctgccccttgtcagatctgtgtctgtgacaatggagccattctctgtgacaagatagaatgccaggatgtgctggactgtgccgaccctgtaacgccccctggggaatgctgtcctgtctgttcacaaacacctggaggtggcaataccaattttggtagaggaagaaagggacaaaagggagaaccaggattagtgcctgttgtaacaggcatacgtggtcgtccaggaccggcaggacctccaggatcacagggaccaagaggagagcgagggccaaaaggaagacctggccctcgtggacctcagggaattgatggagaaccaggtgttcctggtcaacctggtgctccaggacctcctggacatccgtcccacccaggacccgatggcttgagcaggccgttttcagctcaaatggctgggttggatgaaaaatctggacttgggagtcaagtaggactaatgcctggctctgtgggtcctgttggcccaaggggaccacagggtttacaaggacagcaaggtggtgcaggacctacaggacctcctggtgaacctggtgatcctggaccaatgggtccgattggttcacgtggaccagagggccctcctggtaaacctggggaagatggtgaacctggcagaaatggaaatcctggtgaagtgggatttgcaggatctccgggagctcgtggatttcctggggctcctggtcttccaggtctgaagggtcaccgaggacacaaaggtcttgaaggccctaaaggtgaagttggagcacctggttccaagggtgaagctggccccactggtccaatgggtgccatgggtcctctgggtccgaggggaatgccaggagagagagggagacttgggccacagggtgctcctggacaacgaggtgcacatggtatgcctggaaaacctggaccaatgggtcctcttgggataccaggctcttctggttttccaggaaatcctggaatgaagggagaagcaggtcctacaggggcgcgaggccctgaaggtcctcaggggcagagaggtgaaactgggcccccaggtccagttggctctccaggtcttcctggtgcaataggaactgatggtactcctggtgccaaaggcccaacgggctctccaggtacctctggtcctcctggctcagcagggcctcctggatctccaggacctcagggtagcactggtcctcagggaattcgaggccaaccgggtgatccaggagttccaggtttcaaaggagaagctggcccaaaaggggaaccagggccacatggtattcagggtccgataggcccacccggtgaagaaggcaaaagaggtcccagaggtgacccaggaacagttggtcctccagggccagtgggagaaaggggtgctcctggcaatcgtggttttccaggctctgatggtttacctgggccaaagggtgctcaaggagaacggggtcctgtaggttcttcaggacccaaaggaagccagggggatccaggacgtccaggggaacctgggcttccaggtgctcggggtttgacaggaaatcctggtgttcaaggtcctgaaggaaaacttggacctttgggtgcgccaggggaagatggccgtccaggtcctccaggctccataggaatcagagggcagcccgggagcatgggccttccaggccccaaaggtagcagtggtgaccctgggaaacctggagaagcaggaaatgctggagttcctgggcagaggggagctcctggaaaagatggtgaagttggtccttctggtcctgtgggcccgccgggtctagctggtgaaagaggagaacaaggacctccaggccccacaggttttcaggggcttcctggtcctccagggcctcctggagaaggtggaaaaccaggtgatcaaggtgttcctggagatcccggagcagttggcccgttaggacctagaggagaacgaggaaatcctggggaaagaggagaacctgggataactggactccctggtgagaagggaatggctggaggacatggtcctgatggcccaaaaggcagtccaggtccatctgggacccctggagatacaggcccaccaggtcttcaaggtatgccgggagaaagaggaattgcaggaactcctggccccaagggtgacagaggtggcataggagaaaaaggtgctgaaggcacagctggaaatgatggtgcaagaggtcttccaggtcctttgggccctccaggtccggcaggtcctactggagaaaagggtgaacctggtcctcgaggtttagttggccctcctggctcccggggcaatcctggttctcgaggtgaaaatgggccaactggagctgttggttttgccggaccccagggtcctgacggacagcctggagtaaaaggtgaacctggagagccaggacagaagggagatgctggttctcctggaccacaaggtttagcaggatcccctggccctcatggtcctaatggtgttcctggactaaaaggtggtcgaggaacccaaggtccgcctggtgctacaggatttcctggttctgcgggcagagttggacctccaggccctgctggagctccaggacctgcgggacccctaggggaacccgggaaggagggacctccaggtcttcgtggggaccctggctctcatgggcgtgtgggagatcgaggaccagctggcccccctggtggcccaggagacaaaggggacccaggagaagatgggcaacctggtccagatggcccccctggtccagctggaacgaccgggcagagaggaattgttggcatgcctgggcaacgtggagagagaggcatgcccggcctaccaggcccagcgggaacaccaggaaaagtaggaccaactggtgcaacaggagataaaggtccacctggacctgtggggcccccaggctccaatggtcctgtaggggaacctggaccagaaggtccagctggcaatgatggtaccccaggacgggatggtgctgttggagaacgtggtgatcgtggagaccctgggcctgcaggtctgccaggctctcagggtgcccctggaactcctggccctgtgggtgctccaggagatgcaggacaaagaggagatccgggttctcggggtcctataggaccacctggtcgagctgggaaacgtggattacctggaccccaaggacctcgtggtgacaaaggtgatcatggagaccgaggtgacagaggtcagaagggccacagaggctttactggtcttcagggtcttcctggccctcctggtccaaatggtgaacaaggaagtgctggaatccctggaccatttggcccaagaggtcctccaggcccagttggtccttcaggtaaagaaggaaaccctgggccacttgggccaattggacctccaggtgtacgaggcagtgtaggagaagcaggacctgagggccctcctggtgagcctggcccacctggccctccgggtccccctggccaccttacagctgctcttggggatatcatggggcactatgatgaaagcatgccagatccacttcctgagtttactgaagatcaggcggctcctgatgacaaaaacaaaacggacccaggggttcatgctaccctgaagtcactcagtagtcagattgaaaccatgcgcagccccgatggctcgaaaaagcacccagcccgcacgtgtgatgacctaaagctttgccattccgcaaagcagagtggtgaatactggattgatcctaaccaaggatctgttgaagatgcaatcaaagtttactgcaacatggaaacaggagaaacatgtatttcagcaaacccatccagtgtaccacgtaaaacctggtgggccagtaaatctcctgacaataaacctgtttggtatggtcttgatatgaacagagggtctcagttcgcttatggagaccaccaatcacctaatacagccattactcagatgacttttttgcgccttttatcaaaagaagcctcccagaacatcacttacatctgtaaaaacagtgtaggatacatggacgatcaagctaagaacctcaaaaaagctgtggttctcaaaggggcaaatgacttagatatcaaagcagagggaaatattagattccggtatatcgttcttcaagacacttgctctaagcggaatggaaatgtgggcaagactgtctttgaatatagaacacagaatgtggcacgcttgcccatcatagatcttgctcctgtggatgttggcggcacagaccaggaattcggcgttgaaattgggccagtttgttttgtgtaaagtaagccaagacacatcgacaatgagcaccaccatcaatgaccaccgccattcacaagaactttgactgtttgaagttgatcctgagactcttgaagtaatggctgatcctgcatcagcattgtatatatggtcttaagtgcctggcctccttatccttcagaatatttattttacttacaatcctcaagttttaattgattttaaatatttttcaatacaacagtttaggtttaagatgaccaatgacaatgaccacctttgcagaaagtaaactgattgaataaataaatctccgttttcttcaatttatttcagtgtaatgaaaaagttgcttagtatttatgaggaaattcttcttcctggcaggtagcttaaagagtggggtatatagagccacaacacatgtttattttgcttggctgcagttgaaaaatagaaattagtgcccttttgtgacctctcattccaagattgtcaattaaaaatgagtttaaaatgtttaacttgtgatcgagacctacatgcatgtcttgatattgtgtaactataatagagactctttaaggagaatcttaaaaaaaaaaaaacgtttctcactgtcttaaatagaatttttaaatagtatatattcagtggcattttggagaacaaagtgaatttacttcgacttcttaaatttttgtaaaagactataagtttagacatctttctcattcaaatttaaagatatctttctcctcttgatcaatctatcaatattgatagaagtcacactagtatataccatttaatacatttacactttcttatttaagaagatattgaatgcaaaataattgacatatagaactttacaaacatatgtccaaggactctaaattgagactcttccacatgtacaatctcatcatcctgaagcctataatgaagaaaaagatctagaaactgagttgtggagctgactctaatcaaatgtgatgattggaattagaccatttggcctttgaactttcataggaaaaatgacccaacatttcttagcatgagctacctcatctctagaagctgggatggacttactattcttgtttatattttagatactgaaaggtgctatgcttctgttattattccaagactggagataggcagggctaaaaaggtattattatttttcctttaatgatggtgctaaaattcttcctataaaattccttaaaaataaagatggtttaatcactaccattgtgaaaacataactgttagacttcccgtttctgaaagaaagagcatcgttccaatgcttgttcactgttcctctgtcatactgtatctggaatgctttgtaatacttgcatgcttcttagaccagaacatgtaggtccccttgtgtctcaatactttttttttcttaattgcatttgttggctctattttaatttttttcttttaaaataaacagctgggaccatcccaaaagacaagccatgcatacaactttggtcatgtatctctgcaaagcatcaaattaaatgcacgcttttgtcatgtcagtggtttttgttttgtgaaattcctttgaccatattagatctatttcatttccaatagtgaaaaggagatgtggtggtatactttgtttgccatttgtttaaaagatacaacggataccttctatcatgtatgtactggcttataaatgaaaatctatctacaacattacccacaaaggcaacatgacaccaattatcactgcctctgcccttaaaaatgtcagagtagtattattgataaaaagggcaagcaatagatttttcatgactgaataaactgtaataataaaacatatgtctcaaagtgtatcacatatgaatttagcctaattgttttcagtttcattctcaatatttagtttacaacatcattttcccctaaactggttatattttgacctgtatatcttaaatttgagtatttatatgcctaaatacatgtgtgagttttgtttgacttccaagtccaaactataagattatataagttcatatagatgaatcagaaatatgtggtaatactattaagtcacaaacactaacaatttccaactatagaaataacagttcttatttggattttgggaatgctaccaataaaagcctgcccagacca

SEQ ID NO: 175 - DNA, 5072 bp, Homo sapiens
  source 1..5072: mol_type = other DNA
agcaccacggcagcaggaggtttcggctaagttggaggtactggccacgactgcatgcccgcgcccgccaggtgatacctccgccggtgacccaggggctctgcgacacaaggagtctgcatgtctaagtgctagacatgctcagctttgtggatacgcggactttgttgctgcttgcagtaaccttatgcctagcaacatgccaatctttacaagaggaaactgtaagaaagggcccagccggagatagaggaccacgtggagaaaggggtccaccaggccccccaggcagagatggtgaagatggtcccacaggccctcctggtccacctggtcctcctggcccccctggtctcggtgggaactttgctgctcagtatgatggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgtgttggtgcccctggcccagctggtgcccgtggcagtgatggaagtgtgggtcccgtgggtcctgctggtcccattgggtctgctggccctccaggcttcccaggtgcccctggccccaagggtgaaattggagctgttggtaacgctggtcctgctggtcccgccggtccccgtggtgaagtgggtcttccaggcctctccggccccgttggacctcctggtaatcctggagcaaacggccttactggtgccaagggtgctgctggccttcccggcgttgctggggctcccggcctccctggaccccgcggtattcctggccctgttggtgctgccggtgctactggtgccagaggacttgttggtgagcctggtccagctggctccaaaggagagagcggtaacaagggtgagcccggctctgctgggccccaaggtcctcctggtcccagtggtgaagaaggaaagagaggccctaatggggaagctggatctgccggccctccaggacctcctgggctgagaggtagtcctggttctcgtggtcttcctggagctgatggcagagctggcgtcatgggccctcctggtagtcgtggtgcaagtggccctgctggagtccgaggacctaatggagatgctggtcgccctggggagcctggtctcatgggacccagaggtcttcctggttcccctggaaatatcggccccgctggaaaagaaggtcctgtcggcctccctggcatcgacggcaggcctggcccaattggcccagctggagcaagaggagagcctggcaacattggattccctggacccaaaggccccactggtgatcctggcaaaaacggtgataaaggtcatgctggtcttgctggtgctcggggtgctccaggtcctgatggaaacaatggtgctcagggacctcctggaccacagggtgttcaaggtggaaaaggtgaacagggtccccctggtcctccaggcttccagggtctgcctggcccctcaggtcccgctggtgaagttggcaaaccaggagaaaggggtctccatggtgagtttggtctccctggtcctgctggtccaagaggggaacgcggtcccccaggtgagagtggtgctgccggtcctactggtcctattggaagccgaggtccttctggacccccagggcctgatggaaacaagggtgaacctggtgtggttggtgctgtgggcactgctggtccatctggtcctagtggactcccaggagagaggggtgctgctggcatacctggaggcaagggagaaaagggtgaacctggtctcagaggtgaaattggtaaccctggcagagatggtgctcgtggtgctcctggtgctgtaggtgcccctggtcctgctggagccacaggtgaccggggcgaagctggggctgctggtcctgctggtcctgctggtcctcggggaagccctggtgaacgtggtgaggtcggtcctgctggccccaatggatttgctggtcctgctggtgctgctggtcaacctggtgctaaaggagaaagaggagccaaagggcctaagggtgaaaacggtgttgttggtcccacaggccccgttggagctgctggcccagctggtccaaatggtccccccggtcctgctggaagtcgtggtgatggaggcccccctggtatgactggtttccctggtgctgctggacggactggtcccccaggaccctctggtatttctggccctcctggtccccctggtcctgctgggaaagaagggcttcgtggtcctcgtggtgaccaaggtccagttggccgaactggagaagtaggtgcagttggtccccctggcttcgctggtgagaagggtccctctggagaggctggtactgctggacctcctggcactccaggtcctcagggtcttcttggtgctcctggtattctgggtctccctggctcgagaggtgaacgtggtctaccaggtgttgctggtgctgtgggtgaacctggtcctcttggcattgccggccctcctggggcccgtggtcctcctggtgctgtgggtagtcctggagtcaacggtgctcctggtgaagctggtcgtgatggcaaccctgggaacgatggtcccccaggtcgcgatggtcaacccggacacaagggagagcgcggttaccctggcaatattggtcccgttggtgctgcaggtgcacctggtcctcatggccccgtgggtcctgctggcaaacatggaaaccgtggtgaaactggtccttctggtcctgttggtcctgctggtgctgttggcccaagaggtcctagtggcccacaaggcattcgtggcgataagggagagcccggtgaaaaggggcccagaggtcttcctggcttaaagggacacaatggattgcaaggtctgcctggtatcgctggtcaccatggtgatcaaggtgctcctggctccgtgggtcctgctggtcctaggggccctgctggtccttctggccctgctggaaaagatggtcgcactggacatcctggtacagttggacctgctggcattcgaggccctcagggtcaccaaggccctgctggcccccctggtccccctggccctcctggacctccaggtgtaagcggtggtggttatgactttggttacgatggagacttctacagggctgaccagcctcgctcagcaccttctctcagacccaaggactatgaagttgatgctactctgaagtctctcaacaaccagattgagacccttcttactcctgaaggctctagaaagaacccagctcgcacatgccgtgacttgagactcagccacccagagtggagcagtggttactactggattgaccctaaccaaggatgcactatggatgctatcaaagtatactgtgatttctctactggcgaaacctgtatccgggcccaacctgaaaacatcccagccaagaactggtataggagctccaaggacaagaaacacgtctggctaggagaaactatcaatgctggcagccagtttgaatataatgtagaaggagtgacttccaaggaaatggctacccaacttgccttcatgcgcctgctggccaactatgcctctcagaacatcacctaccactgcaagaacagcattgcatacatggatgaggagactggcaacctgaaaaaggctgtcattctacagggctctaatgatgttgaacttgttgctgagggcaacagcaggttcacttacactgttcttgtagatggctgctctaaaaagacaaatgaatggggaaagacaatcattgaatacaaaacaaataagccatcacgcctgcccttccttgatattgcacctttggacatcggtggtgctgaccaggaattctttgtggacattggcccagtctgtttcaaataaatgaactcaatctaaattaaaaaagaaagaaatttgaaaaaactttctctttgccatttcttcttcttcttttttaactgaaagctgaatccttccatttcttctgcacatctacttgcttaaattgtgggcaaaagagaaaaagaaggattgatcagagcattgtgcaatacagtttcattaactccttcccccgctcccccaaaaatttgaatttttttttcaacactcttacacctgttatggaaaatgtcaacctttgtaagaaaaccaaaataaaaattgaaaaataaaaaccataaacatttgcaccacttgtggcttttgaatatcttccacagagggaagtttaaaacccaaacttccaaaggtttaaactacctcaaaacactttcccatgagtgtgatccacattgttaggtgctgacctagacagagatgaactgaggtccttgttttgttttgttcataatacaaaggtgctaattaatagtatttcagatacttgaagaatgttgatggtgctagaagaatttgagaagaaatactcctgtattgagttgtatcgtgtggtgtattttttaaaaaatttgatttagcattcatattttccatcttattcccaattaaaagtatgcagattatttgcccaaatcttcttcagattcagcatttgttctttgccagtctcattttcatcttcttccatggttccacagaagctttgtttcttgggcaagcagaaaaattaaattgtacctattttgtatatgtgagatgtttaaataaattgtgaaaaaaatgaaataaagcatgtttggttttccaaaagaa

SEQ ID NO: 176 - DNA, 6540 bp, Homo sapiens
  source 1..6540: mol_type = other DNA
aggtctccgcttggagccgccgcacccgggacggtgcgtagcgctggaagtccggccttccgagagctagctgtccgccgcggcccccgcacgccgggcagccgtccctcgccgcctcgggcgcgccaccatggggccccggctcagcgtctggctgctgctgctgcccgccgcccttctgctccacgaggagcacagccgggccgctgcgaagggtggctgtgctggctctggctgtggcaaatgtgactgccatggagtgaagggacaaaagggtgaaagaggcctcccggggttacaaggtgtcattgggtttcctggaatgcaaggacctgaggggccacagggaccaccaggacaaaagggtgatactggagaaccaggactacctggaacaaaagggacaagaggacctccgggagcatctggctaccctggaaacccaggacttcccggaattcctggccaagacggcccgccaggccccccaggtattccaggatgcaatggcacaaagggggagagagggccgctcgggcctcctggcttgcctggtttcgctggaaatcccggaccaccaggcttaccagggatgaagggtgatccaggtgagatacttggccatgtgcccgggatgctgttgaaaggtgaaagaggatttcccggaatcccagggactccaggcccaccaggactgccagggcttcaaggtcctgttgggcctccaggatttaccggaccaccaggtcccccaggccctcccggccctccaggtgaaaagggacaaatgggcttaagttttcaaggaccaaaaggtgacaagggtgaccaaggggtcagtgggcctccaggagtaccaggacaagctcaagttcaagaaaaaggagacttcgccaccaagggagaaaagggccaaaaaggtgaacctggatttcaggggatgccaggggtcggagagaaaggtgaacccggaaaaccaggacccagaggcaaacccggaaaagatggtgacaaaggggaaaaagggagtcccggttttcctggtgaacccgggtacccaggactcataggccgccagggcccgcagggagaaaagggtgaagcaggtcctcctggcccacctggaattgttataggcacaggacctttgggagaaaaaggagagaggggctaccctggaactccggggccaagaggagagccaggcccaaaaggtttcccaggactaccaggccaacccggacctccaggcctccctgtacctgggcaggctggtgcccctggcttccctggtgaaagaggagaaaaaggtgaccgaggatttcctggtacatctctgccaggaccaagtggaagagatgggctcccgggtcctcctggttcccctgggccccctgggcagcctggctacacaaatggaattgtggaatgtcagcccggacctccaggtgaccagggtcctcctggaattccagggcagccaggatttataggcgaaattggagagaaaggtcaaaaaggagagagttgcctcatctgtgatatagacggatatcgggggcctcccgggccacagggacccccgggagaaataggtttcccagggcagccaggggccaagggcgacagaggtttgcctggcagagatggtgttgcaggagtgccaggccctcaaggtacaccagggctgataggccagccaggagccaagggggagcctggtgagttttatttcgacttgcggctcaaaggtgacaaaggagacccaggctttccaggacagcccggcatgccagggagagcgggttctcctggaagagatggccatccgggtcttcctggccccaagggctcgccgggttctgtaggattgaaaggagagcgtggcccccctggaggagttggattcccaggcagtcgtggtgacaccggcccccctgggcctccaggatatggtcctgctggtcccattggtgacaaaggacaagcaggctttcctggaggccctggatccccaggcctgccaggtccaaagggtgaaccaggaaaaattgttcctttaccaggcccccctggagcagaaggactgccggggtccccaggcttcccaggtccccaaggagaccgaggctttcccggaaccccaggaaggccaggcctgccaggagagaagggcgctgtgggccagccaggcattggatttccagggccccccggccccaaaggtgttgacggcttacctggagacatggggccaccggggactccaggtcgcccgggatttaatggcttacctgggaacccaggtgtgcagggccagaagggagagcctggagttggtctaccgggactcaaaggtttgccaggtcttcccggcattcctggcacacccggggagaaggggagcattggggtaccaggcgttcctggagaacatggagcgatcggaccccctgggcttcaggggatcagaggtgaaccgggacctcctggattgccaggctccgtggggtctccaggagttccaggaataggcccccctggagctaggggtccccctggaggacagggaccaccggggttgtcaggccctcctggaataaaaggagagaagggtttccccggattccctggactggacatgccgggccctaaaggagataaaggggctcaaggactccctggcataacgggacagtcggggctccctggccttcctggacagcagggggctcctgggattcctgggtttccaggttccaagggagaaatgggcgtcatggggacccccgggcagccgggctcaccaggaccagtgggtgctcctggattaccgggtgaaaaaggggaccatggctttccgggctcctcaggacccaggggagaccctggcttgaaaggtgataagggggatgtcggtctccctggcaagcctggctccatggataaggtggacatgggcagcatgaagggccagaaaggagaccaaggagagaaaggacaaattggaccaattggtgagaagggatcccgaggagaccctgggaccccaggagtgcctggaaaggacgggcaggcaggacagcctgggcagccaggacctaaaggtgatccaggtataagtggaaccccaggtgctccaggacttccgggaccaaaaggatctgttggtggaatgggcttgccaggaacacctggagagaaaggtgtgcctggcatccctggcccacaaggttcacctggcttacctggagacaaaggtgcaaaaggagagaaagggcaggcaggcccacctggcataggcatcccagggctgcgaggtgaaaagggagatcaagggatagcgggtttcccaggaagccctggagagaagggagaaaaaggaagcattgggatcccaggaatgccagggtccccaggccttaaagggtctcccgggagtgttggctatccaggaagtcctgggctacctggagaaaaaggtgacaaaggcctcccaggattggatggcatccctggtgtcaaaggagaagcaggtcttcctgggactcctggccccacaggcccagctggccagaaaggggagccaggcagtgatggaatcccggggtcagcaggagagaagggtgaaccaggtctaccaggaagaggattcccagggtttccaggggccaaaggagacaaaggttcaaagggtgaggtgggtttcccaggattagccgggagcccaggaattcctggatccaaaggagagcaaggattcatgggtcctccggggccccagggacagccggggttaccgggatccccaggccatgccacggaggggcccaaaggagaccgcggacctcagggccagcctggcctgccaggacttccgggacccatggggcctccagggcttcctgggattgatggagttaaaggtgacaaaggaaatccaggctggccaggagcacccggtgtcccagggcccaagggagaccctggattccagggcatgcctggtattggtggctctccaggaatcacaggctctaagggtgatatggggcctccaggagttccaggatttcaaggtccaaaaggtcttcctggcctccagggaattaaaggtgatcaaggcgatcaaggcgtcccgggagctaaaggtctcccgggtcctcctggccccccaggtccttacgacatcatcaaaggggagcccgggctccctggtcctgagggccccccagggctgaaagggcttcagggactgccaggcccgaaaggccagcaaggtgttacaggattggtgggtatacctggacctccaggtattcctgggtttgacggtgcccctggccagaaaggagagatgggacctgccgggcctactggtccaagaggatttccaggtccaccaggccccgatgggttgccaggatccatggggcccccaggcaccccatctgttgatcacggcttccttgtgaccaggcatagtcaaacaatagatgacccacagtgtccttctgggaccaaaattctttaccacgggtactctttgctctacgtgcaaggcaatgaacgggcccatggccaggacttgggcacggccggcagctgcctgcgcaagttcagcacaatgcccttcctgttctgcaatattaacaacgtgtgcaactttgcatcacgaaatgactactcgtactggctgtccacccctgagcccatgcccatgtcaatggcacccatcacgggggaaaacataagaccatttattagtaggtgtgctgtgtgtgaggcgcctgccatggtgatggccgtgcacagccagaccattcagatcccaccgtgccccagcgggtggtcctcgctgtggatcggctactcttttgtgatgcacaccagcgctggtgcagaaggctctggccaagccctggcgtcccccggctcctgcctggaggagtttagaagtgcgccattcatcgagtgtcacggccgtgggacctgcaattactacgcaaacgcttacagcttttggctcgccaccatagagaggagcgagatgttcaagaagcctacgccgtccaccttgaaggcaggggagctgcgcacgcacgtcagccgctgccaagtctgtatgagaagaacataatgaagcctgactcagctaatgtcacaacatggtgctacttcttcttctttttgttaacagcaacgaaccctagaaatatatcctgtgtacctcactgtccaatatgaaaaccgtaaagtgccttataggaatttgcgtaactaacacaccctgcttcattgacctctacttgctgaaggagaaaaagacagcgataagctttcaatagtggcataccaaatggcacttttgatgaaataaaatatcaatattttctgcaatccaatgcactgatgtgtgaagtgagaactccatcagaaaaccaaagggtgctaggaggtgtgggtgccttccatactgtttgcccattttcattcttgtattataattaattttctacccccagagataaatgtttgtttatatcactgtctagctgtttcaaaatttaggtcccttggtctgtacaaataatagcaatgtaaaaatggttttttgaacctccaaatggaattacagactcagtagccatatcttccaaccccccagtataaatttctgtctttctgctatgtgtggtactttgcagctgcttttgcagaaatcacaattttcctgtggaataaagatggtccaaaaatagtcaaaaattaaatatatatatatattagtaatttatatagatgtcagcaattaggcagatcaaggtttagtttaacttccactgttaaaataaagcttacatagttttcttcctttgaaagactgtgctgtcctttaacataggtttttaaagactaggatattgaatgtgaaacatccgttttcattgttcacttctaaaccaaaaattatgtgttgccaaaaccaaacccaggttcatgaatatggtgtctattatagtgaaacatgtactttgagcttattgtttttattctgtattaaatattttcagggttttaaacactaatcacaaactgaatgacttgacttcaaaagcaacaaccttaaaggccgtcatttcattagtattcctcattctgcatcctggcttgaaaaacagctctgttgaatcacagtatcagtattttcacacgtaagcacattcgggccatttccgtggtttctcatgagctgtgttcacagacctcagcagggcatcgcatggaccgcaggagggcagattcggaccactaggcctgaaatgacatttcactaaaagtctccaaaacatttctaagactactaaggccttttatgtaatttctttaaatgtgtatttcttaagaattcaaatttgtaataaaactatttgtataaaaattaagcttttattaatttgttgctagtattgccacagacgcattaaaagaaacttactgcacaagctgctaataaatttgtaagctttgcatacctta

SEQ ID NO: 177 - DNA, 6446 bp, Homo sapiens
  source 1..6446: mol_type = other DNA
gagtgtggctgcagtgcgccgggacaccagggctccgcgctccgcactcaagaggctcccgcgtcccaacccctcgcgcccgcgcgttcgcggatccaggccgaggaccgaaaggggccgcccgagcccccggggccggcgcccagagagcccagcaaggccggccgccctgccggtgtgccgccggcgggtgcttctggaagggccaatgcgttcgggcagcagcccctgaagccgagcccgaggctaagtgggactgaccggggcccagagtggacgaaccgccagcatggggagagaccagcgcgcggtggccggccctgccctacggcggtggctgctgctggggacagtgaccgtggggttcctcgcccagagcgtcttggcgggtgtgaagaagtttgatgtgccgtgtggaggaagagattgcagtgggggctgccagtgctaccctgagaaaggtggacgtggtcagcctgggccagtgggcccccaggggtacaatgggccaccaggattacaaggattcccgggactgcagggacgtaaaggagacaagggtgaaaggggagcccccggagtaacgggacccaagggcgacgtgggagcaagaggcgtttctggattccctggtgccgatggaattcctggacacccggggcaaggtgggcccaggggaaggccgggctacgatggctgcaacggaacccagggagactcaggtccacaggggccccccggctctgaggggttcaccgggcctcccgggccccaaggaccaaaagggcagaaaggtgagccttatgcactgcctaaagaggagcgcgacagatatcggggtgaacctggagagcctggattggtcggtttccagggacctcccggccgccctgggcatgtgggacagatgggtccagttggagctccagggagaccaggaccacctggaccccctggaccaaaaggacagcaaggcaacagaggacttggtttctacggagttaagggtgaaaagggtgacgtagggcagccgggacccaacgggattccatcagacaccctccaccccatcatcgcgcccacaggagtcaccttccacccagatcagtacaagggtgaaaaaggcagtgagggggaaccaggaataagaggcatttccttgaagggagaagaaggaatcatgggctttcctggactgaggggttaccctggcttgagtggtgaaaaaggatcaccaggacagaagggaagccgaggcctggatggctatcaagggcctgatggaccccggggacccaagggagaagccggagacccagggccccctggactacctgcctactcccctcacccttccctagcaaaaggtgccagaggtgacccgggattcccaggggcccaaggggagccaggaagccagggtgagccaggagacccgggcctcccaggtccccctggcctctccatcggagatggagatcagaggagaggcctgccgggtgagatgggacccaagggcttcatcggagaccccggcatccctgcgctctacgggggcccacctggacctgatggaaagcgagggcctccaggaccccccgggctccctggaccacctggacctgatggcttcctgtttgggctgaaaggagcaaaaggaagagcaggcttccctgggcttcccggctcccctggagcccgcggaccaaaggggtggaaaggtgacgctggggaatgcagatgtacagaaggcgacgaagctatcaaaggtcttccgggactgccaggacccaagggcttcgcaggcatcaacggggagccggggaggaaaggggacagaggagaccccggccaacacggcctccctgggttcccagggctcaagggagtgcctggcaacattggtgctcccggacccaaaggagcaaaaggagattccagaacaatcacaaccaaaggtgagcggggacagcccggcgtcccaggtgtgcccgggatgaaaggtgacgatggcagcccaggccgcgatgggctcgatggattccccggcctcccaggccctcccggtgatggcatcaagggccctccaggggacccaggctatccaggaatacctggaacgaagggtactccaggagaaatgggccccccaggactgggccttcccggcctcaaaggccaacgtggtttccctggagacgccggcttacctggaccaccaggcttcctgggccctcctggccccgcagggaccccaggacaaatagattgtgacacagatgtgaaaagggccgttggaggtgacagacaggaggccatccagccaggttgcataggagggcccaagggattgccaggcctgccaggacccccaggccccacaggtgccaaaggcctccgaggaatcccaggcttcgcaggagctgatggaggaccagggcccaggggcttgccaggagacgcaggtcgtgaagggttcccaggacccccagggttcataggaccccgaggatccaaaggtgcagtgggcctccctggcccagatggatccccaggtcccatcggcctgccagggccagatgggccccctggggaaaggggcctccctggagaagtcctgggagctcagcccgggccacggggagatgctggtgtgcctggacagcctgggcttaaaggccttcccggagacagaggcccccctggattcagaggaagccaagggatgcctgggatgccagggctgaagggccagccaggcctcccaggaccttccggccagccaggcctgtatgggcctccaggactgcatggattcccaggagctcctggccaagaggggcccttggggctgccaggaatcccaggccgtgaaggtctgcctggtgatagaggggaccctggggacacaggcgctcctggccctgtgggcatgaaaggtctctctggtgacagaggagatgctggcttcacaggggagcaaggccatccaggaagccctggatttaaaggaattgatggaatgcctgggacccccgggctaaaaggagatagaggctcacctgggatggatggtttccaaggcatgcctggactcaaagggagacccgggtttccagggagcaaaggcgaggctggatttttcggaatacccggtctgaagggtctggctggtgagccaggttttaaaggcagccgaggggaccctgggcccccaggaccacctcctgtcatcctgccaggaatgaaagacattaaaggagagaaaggagatgaagggcctatggggctgaaaggatacctgggcgcaaaaggtatccaaggaatgccaggcatcccagggctgtcaggaatccctgggctgcctgggaggcccggccacatcaaaggagtcaagggagacatcggagtccccggcatccccggtttgccaggattccctggggtggctggcccccctggaattacgggattcccaggattcataggaagccggggtgacaaaggtgccccagggagagcaggcctgtatggcgagattggcgcgactggtgatttcggtgacatcggggacactataaatttaccaggaagaccaggcctgaagggggagcggggcaccactggaataccaggtctgaagggattctttggagagaagggaacagaaggtgacatcggcttccctgggataacaggcgtgactggagtccaaggccctcctggacttaaaggacaaacaggctttccagggctgactgggcctccagggtcgcagggagagctggggcggattggactgcctggtggcaaaggagatgatggctggccgggagctccgggcttaccaggttttccgggactccgtgggatccgcggcttacacggcttgccaggcaccaagggctttccaggatccccaggttctgacatccacggagacccaggcttcccaggccctcctggggaaagaggtgacccaggagaggccaacacccttccaggccctgtgggagtcccaggacagaaaggagaccaaggagctccaggggaacgaggcccacctgggagcccaggacttcaggggttccctggtatcacacccccttccaacatctctggggcacctggtgacaaaggggcgccagggatatttggcctgaaaggttatcggggcccaccagggccaccaggttctgctgctcttcctggaagcaaaggtgacacagggaacccaggagctccaggaaccccagggaccaaaggatgggccggggactccgggccccagggcaggcctggtgtgtttggtctcccaggagaaaaagggcccaggggtgaacaaggcttcatggggaacactggacccactggggcggtgggcgacagaggccccaagggacccaagggagacccaggattccctggtgcccccgggactgtgggagcccccgggattgcaggaatcccccagaagattgccgtccaaccagggacagtgggtccccaggggaggcgaggcccccctggggcaccgggggagatggggccccagggcccccccggagaaccaggtttccgtggggctccagggaaagctgggccccaaggaagaggtggtgtgtctgctgttcccggcttccggggagatgaaggacccataggccaccaggggccgattggccaagaaggtgcaccaggccgtccagggagcccgggcctgccgggtatgccaggccgcagcgtcagcatcggctacctcctggtgaagcacagccagacggaccaggagcccatgtgcccagtgggcatgaacaaactctggagtggatacagcctgctgtacttcgagggccaggagaaggcgcacaaccaggacctggggctggcgggctcctgcctggcgcggttcagcaccatgcccttcctgtactgcaaccctggtgatgtctgctactatgccagccggaacgacaagtcctactggctctctaccactgcgccgctgcccatgatgcccgtggccgaggacgagatcaagccctacatcagccgctgttctgtgtgtgaggccccggccatcgccatcgcggtccacagtcaggatgtctccatcccacactgcccagctgggtggcggagtttgtggatcggatattccttcctcatgcacacggcggcgggagacgaaggcggtggccaatcactggtgtcaccgggcagctgtctagaggacttccgcgccacaccattcatcgaatgcaatggaggccgcggcacctgccactactacgccaacaagtacagcttctggctgaccaccattcccgagcagagcttccagggctcgccctccgccgacacgctcaaggccggcctcatccgcacacacatcagccgctgccaggtgtgcatgaagaacctgtgagccggcgcgtgccaggaagggccattttggtgcttattcttaacttattacctcaggtgccaacccaaaaattggttttatttttttcttaaaaaaaaaaaagtctaccaaaggaatttgcatccagcagcagcacttagacctgccagccactgtcaccgagcgggtgcaagcactcggggtccctggagggcaagccctgcccacagaaagccaggagcagccctggcccccatcagccctgctagacgcaccgcctgaaggcacagctaaccacttcgcacacacccatgtaaccactgcactttccaatgccacagacaactcacattgttcaactcccttctcggggtgggacagacgagacaacagcacacaggcagccagccgtggccagaggctcgaggggctcagggcctcaggcacccgtccccacacgagggccccgtgggtgggcctggccctgctttctacgccaatgttatgccagctccatgttctcccaaataccgttgatgtgaattattttaaaggcaaaaccgtgctctttattttaaaaaacactgataatcacactgcggtaggtcattcttttgccacatccctatagaccactgggtttggcaaaactcaggcagaagtggagacctttctagacatcattgtcagccttgctacttgaaggtacaccccatagggtcggaggtgctgtccccactgccccacgttgtccctgagatttaacccctccactgctgggggtgagctgtactcttctgactgccccctcctgtgtaacgactacaaaataaaacttggttctgaatatttttaaaccccgagttgttgaccgccttaatctcgtgtccatagagcaaaacgtctgctcagatggatgcgaggcacagcgtccgcccacgctgctgtttttaatccatctcagtagagttgaacccattcgtggtattacagccatttctcggggaatgtgtttgtttataactcactaatgcttaca

SEQ ID NO: 178 - DNA, 2868 bp, Homo sapiens
  source 1..2868: mol_type = other DNA
ggctgcgctccgagctgcggagtccgggactggagctgcccgggcgggttcgcgccccgaaggctgagagctggcgctgctcgtgccctgtgtgccagacggcggagctccgcggccggaccccgcggccccgctttgctgccgactggagtttgggggaagaaactctcctgcgccccagaggatttcttcctcggcgaagggacagcgaaagatgagggtggcaggaagagaagggcgctttctgtctgccggggtcgcagcgcgagagggcagtgccatgttcctctccatcctagtggcgctgtgcctgtggctgcacctggcgctgggcgtgcgcggcgcgccctgcgaggcggtgcgcatccctatgtgccggcacatgccctggaacatcacgcggatgcccaaccacctgcaccacagcacgcaggagaacgccatcctggccatcgagcagtacgaggagctggtggacgtgaactgcagcgccgtgctgcgcttcttcctctgtgccatgtacgcgcccatttgcaccctggagttcctgcacgaccctatcaagccgtgcaagtcggtgtgccaacgcgcgcgcgacgactgcgagcccctcatgaagatgtacaaccacagctggcccgaaagcctggcctgcgacgagctgcctgtctatgaccgtggcgtgtgcatctcgcctgaagccatcgtcacggacctcccggaggatgttaagtggatagacatcacaccagacatgatggtacaggaaaggcctcttgatgttgactgtaaacgcctaagccccgatcggtgcaagtgtaaaaaggtgaagccaactttggcaacgtatctcagcaaaaactacagctatgttattcatgccaaaataaaagctgtgcagaggagtggctgcaatgaggtcacaacggtggtggatgtaaaagagatcttcaagtcctcatcacccatccctcgaactcaagtcccgctcattacaaattcttcttgccagtgtccacacatcctgccccatcaagatgttctcatcatgtgttacgagtggcgctcaaggatgatgcttcttgaaaattgcttagttgaaaaatggagagatcagcttagtaaaagatccatacagtgggaagagaggctgcaggaacagcggagaacagttcaggacaagaagaaaacagccgggcgcaccagtcgtagtaatccccccaaaccaaagggaaagcctcctgctcccaaaccagccagtcccaagaagaacattaaaactaggagtgcccagaagagaacaaacccgaaaagagtgtgagctaactagtttccaaagcggagacttccgacttccttacaggatgaggctgggcattgcctgggacagcctatgtaaggccatgtgccccttgccctaacaactcactgcagtgctcttcatagacacatcttgcagcatttttcttaaggctatgcttcagtttttctttgtaagccatcacaagccatagtggtaggtttgccctttggtacagaaggtgagttaaagctggtggaaaaggcttattgcattgcattcagagtaacctgtgtgcatactctagaagagtagggaaaataatgcttgttacaattcgacctaatatgtgcattgtaaaataaatgccatatttcaaacaaaacacgtaatttttttacagtatgttttattaccttttgatatctgttgttgcaatgttagtgatgttttaaaatgtgatcgaaaatataatgcttctaagaaggaacagtagtggaatgaatgtctaaaagatctttatgtgtttatggtctgcagaaggatttttgtgatgaaaggggattttttgaaaaatctagagaagtagcatatggaaaactataatgtgtcttttttacaatgacttcagctctgtttttagctagaaactctaaaaacaaaaataataataaagaaaaataaataaaaaggagaggcagacaatgtctggattcctgttttttggttacctgatttcatgatcatgatgcttcttgtcaacaccctcttaagcagcaccagaaacagtgagtttgtctgtaccattaggagttaggtactaattagttggctaatgctcaagtattttatacccacaagagaggtatgtcactcatcttacttcccaggacatccaccctgagaataatttgacaagcttaaaaatggccttcatgtgagtgccaaattttgttttcttcatttaaatattttctttgcctaaatacatgtgagaggagttaaatataaatgtacagagaggaaagttgaggttccacctctgaaatgagaattacttgacagttgggatactttaatcagaaaaaaagaacttatcttgcagcattttatcaacaaatttcataattgtggacaattggaggcatttattttaaaaaacaattttattggccttttgctaacacagtaagcatgtattctctataaggcattcaataaatgcacaacgcccaaaggaaataaaatcctatctaatcctactctccactacacagaggtaatcactattagtattttggcatattattctccaggtgtttcttatgcacttataaaatgatttgaacaaataaaactaggaacctgctatacatgtgtttcataacctgcctcctttgcttggccctttattgagataagttttcctgtcaagaaagcagaaaccatctcatttctaacagctgtgttatattccatagtatgcattactcaacaaactgttgtgctattggatacttaggtggtttcttcactgacaatactgaataaacatctcaatagtcaaa

SEQ ID NO: 179 - DNA, 7437 bp, Homo sapiens
  source 1..7437: mol_type = other DNA
agaaacagccctcaagtttcatcacagggatgctaaaatatattctccagcttcagcaatgacctagttcactgaagggcggggaaaaaatgctcttcttccagaacttccccgggcatggaatttcccctcttaggaagagatgaactttctggaagcagacccatctatgctttgacctgatcatggaagaccctgagccctataaactcctttgaatccctgtcccgggaacagggacacccaatcaatgcctcacacacagcctggggaatccacagctgagccagccctcctacaacatttcttggacaaagagtactgaaaaatttggtgcggcatgaaaggatgaaagactccgaagggccccagaggcccccgctgtgttttttatctacattgctttcccagaaggttcctgagaagtcagacgctgtgcttcgctgcataatatctggtcagcccaagccagaggtaacttggtataagaatggtcaggccatcgatgggagtggcattatttccaactatgaattctttgagaatcagtatattcatgtgttacatctctcttgctgtaccaaaaatgatgctgctgtctatcaaatctcggctaaaaactcttttggaatgatctgttgttctgcttccgttgaggttgagtgctcatcagagaacccacaattgtctcctaacctggaagatgacagggacaggggttggaaacatgaaacagggacacatgaagaagaaagggcaaatcagattgatgagaaggaacatccttataaggaagaagaaagcatctccccgggcactcccaggtcagctgactcctccccctccaaatccaaccattcactctccctccagtcattgggcaatcttgacattagtgtgtccagttctgaaaatcctttgggtgttaaaggaacaaggcacactggagaggcttatgatccaagtaacacagaagaaattgcaaatgggttgctttttcttaattcaagtcatatttatgaaaaacaagacagatgttgccacaagacagtgcattccatggcatcaaagttcacggatggtgacctgaacaatgatggtcctcatgatgaaggcttacgctctagtcagcaaaatcccaaagtacagaaatacattagcttcagcctcccgctatctgaggcaactgcacacatttacccaggtgacagtgccgtggccaacaaacaacccagcccacagctttccagtgaagactctgacagtgactatgaactttgcccagagataaccctaacctacaccgaggagttttcagatgatgacctggagtatctggaatgttctgatgttatgacggattactctaatgcagtttggcaaaggaacctgctggggactgagcatgtttttttattagaaagcgatgacgaagagatggaattcggtgagcattgcctgggtgggtgtgagcatttcctcagtggaatgggttgtgggtctcgggtgtcgggtgacgctgggcctatggttgccactgctggcttctgtggtcatcactcacaaccccaagaagttggggtgaggagcagcagagtctccaagcacggtccctcatccccacaaacagggatgactctcattttgggacctcaccaggatggaacgtcttcagtgacagaacaggggagatataaactccccactgctcccgaggctgctgaaaatgattatccaggaattcaaggagaaaccagagacagccaccaagcaagagaggaatttgccagtgacaatctgctcaacatggatgaatcagtaagagagacagagatgaagctcttgtctggtgagtcagaaaactcagggatgagccagtgttgggagacggcagctgacaagagagtggggggaaaggacttatggagcaagaggggttcaaggaaatctgccagggtgaggcagccgggaatgaagggaaatcccaagaagccgaatgccaacctgagagaaagtacaacagaaggtacccttcatctctgctctgccaaagaatctgctgagcccccactaacccagagtgataaaagagagacttctcacaccacagcagcagcgactggtcggagttcccatgctgatgcaagagaatgtgctatttcaacccaggcagagcaagaagcaaaaacccttcaaacttcaacagactcagtctccaaagaaggcaacacaaattgcaagggagaaggcatgcaagttaatactctatttgaaacaagccaggttccagactggagtgatcctcctcaggtacaagttcaggaaacagtcagagagacaatctcttgcagccagatgccagctttctcagagcctgctggggaggagtccccattcactgggaccacaacaatttccttctcaaacttaggaggggtccacaaggaaaatgcatcattagctcaacactcggaggtcaaaccctgtacctgtggtccacagcatgaagaaaaacaagacagagatggcaacatacctgacaatttcagggaagacctaaaatatgagcagagcatctcagaagccaatgatgagactatgtccccaggtgtgttctcaaggcatctccccaaggatgctcgtgctgacttcagggagcctgtggctgtctctgttgcttcccctgaacccacagatactgccctcaccctggaaaatgtgtgtgatgagccaagggacagagaagcagtgtgtgcaatggagtgttttgaggctggtgaccaaggaacgtgttttgataccatagattctcttgttgggagaccagttgataaatattcgcctcaagaaatttgctctgtagatacggaactggcagaaggtcaaaacaaagtatctgatttatgttcttctaatgacaagacactggaagtcttttttcagacacaagtgtctgagacttcagtgtctacgtgcaaaagcagcaaggacggcaactcagtcatgtcccctctttttaccagtactttcaccttgaacatttcacacacagctagtgaaggtgccacaggagaaaatctagccaaggtggagaattccacctacccactggcctccacagtacatgctggccaggagcagccaagccccagcaactcaggagggcttgatgaaacacagctcctttcttctgagaacaatcctttagtgcaatttaaagaaggaggtgacaagagccccagtcctagtgccgcagacaccacagccacaccagccagttatagttcaattgtgagttttccttgggagaagccaacaacattaactgctaataatgagtgctttcaagcgaccagggagactgaggacacatcaactgttaccattgccaccgaagtccacccagccaaataccttgctgtgtcaattcctgaggacaagcatgcaggtggcactgaggagaggttccctcgtgcatcccatgaaaaggtttcccaatttccttcccaagtgcagttggatcatattttaagtggtgctaccatcaaatctacaaaagagctactttgcagggcacccagtgtgccaggagtcccacaccatgtcctgcagctcccagagggagagggtttctgcagtaattcccctcttcaggttgataacctgtctggagataagagccagactgtggacagagcagactttaggagctatgaagagaatttccaagaaagaggaagtgaaacaaagcagggggtccagcagcagagcctgtcccagcagggttctctttctgcacctgatttccaacaaagtttgcctacgacatctgctgcacaagaggaaagaaacttggtgcccacggcccactcacccgcaagctctagggaaggagcagggcagcgctcaggttgggggacgagggtctccgtggtggctgaaactgctggggaagaagacagtcaggctctgagcaacgttccatctctctctgatatccttttggaagagtctaaagaatatagacctggaaattgggaggcaggcaacaagctgaagattataactctagaggcttccgcttctgaaatctggccaccacgacaactgacaaattctgagagcaaggcatcagacggtggtctcataattcctgacaaggtctgggctgtacctgatagtctaaaggcagatgctgttgtgcctgaattggccccctctgaaatagcagcattggctcacagtccagaggatgctgagtcagcccttgctgatagcagagaaagccataaaggcgaagagcccaccatcagtgtacattggagaagtctttcttcccggggtttcagccaacccagactcctggagtcatccgtggaccctgtagatgaaaaggagttatctgtcacagattcactgtcagcggcttctgaaactggagggaaggaaaatgttaacaatgtgagtcaagaccaggaggaaaaacaactcaagatggatcacactgccttctttaaaaagtttctgacctgccctaaaatcctagagtcctctgtagatcccattgatgagataagtgtgatagagtacaccagggctggaaaaccagagccctctgaaaccacaccacagggcgccagagaagggggtcaatcaaatgacggaaacatgggccacgaagcggaaatccagccggccattttgcaagttccatgtctccagggaaccattctgagtgaaaatagaatcagcagaagccaagaaggcagtatgaagcaggaggctgaacaaattcaacctgaggaggcaaaaactgccatttggcaagtcctgcaacccagcgaaggcggtgaaagaattccaagtggatgtagcataggccaaatacaagaaagcagtgatgggagcttaggggaggctgagcaaagcaaaaaggacaaagcagaattgatttcccccacttcacctctttctagttgtcttccaataatgactcacgcttctcttggggttgacacgcacaactccacaggccaaattcatgacgtccctgaaaatgacatagttgagcccagaaagcgtcagtatgtgtttcctgtttcacagaaaaggggaactattgagaatgagcgtgggaaacctttgccctcttctcctgatcttaccaggttcccttgtacttcatctcctgaaggaaatgtcacagactttttgataagccacaaaatggaggaacctaaaatagaggtgcttcaaattggggaaaccaaacccccaagctcatctagctcctcagcgaagaccttggcatttatttcaggagaacgtgagttagagaaagcccctaaattactgcaggatccatgtcaaaagggcaccctgggctgtgcgaaaaagtccagggagagagagaagtccctggaagcccgagcaggcaaatcgccagggaccctcacagcagtgacggggtcagaggaggtcaagaggaagccagaagccccaggcagtggacatttagctgagggagtaaagaagaaaattttgtccagggtggcagcactgaggctgaaactggaagaaaaggaaaatatcagaaagaactcagcctttcttaaaaagatgcccaaactcgaaacatcattatcacacacagaagagaaacaagacccaaaaaagccatcttgcaaaagagaaggaagagctccagtattactgaaaaagatccaagctgagatgttccctgaacactctggaaatgtaaaattaagctgccaatttgcagaaattcatgaagattctactatctgctggacaaaagattcaaagtccatagcccaagtgcagagaagtgcaggggacaactccactgtttcctttgccatcgtgcaagccagtccgaaggaccagggactctattactgctgcatcaagaacagctacggaaaagtgactgctgaatttaacctcacagctgaagttctcaaacagctgtcaagtcgccaggatactaaaggatgtgaagagattgaattcagccaactcatcttcaaagaagacttcctccatgacagctactttgggggccgcctgcgtggtcagatcgccacggaggagctgcactttggagaaggggttcaccgcaaagccttccgcagcacagtgatgcacggcctcatgcctgtcttcaaacctggccatgcctgtgtgcttaaggtgcacaatgccattgcctatgggaccagaaataatgatgagctcatccaaaggaactacaaactcgctgcccaggaatgctatgttcaaaatactgccaggtattatgccaagatctacgctgctgaagcacagcctctggaaggctttggagaagtacctgagatcattcctatttttcttatccatcggcctgagaacaatatcccgtatgctacagtggaggaggagctgattggagaatttgtgaagtattccatcagggatgggaaagaaataaacttcttgagaagagaatcagaagctggtcagaaatgttgcaccttccagcactgggtgtaccagaaaacaagtggctgcctcctggtgacggacatgcaaggtgtaggaatgaagctaactgacgttggcatagcaacgctggctaaagggtacaagggatttaaaggcaactgttccatgaccttcattgatcagtttaaagcactacaccagtgtaacaagtattgcaaaatgctgggactgaaatcccttcaaaacaacaaccagaaacagaagcagccgagcattgggaaaagcaaagttcaaacaaactctatgacaataaagaaggcagggcctgagaccccaggcgaaaagaaaacctaacgtccctgggtaacctaatggccactggctagcagcacacaatctcgccagggaaaatctgaggccacacaggagagaatatacagcctgcagagagtgcgtggcaatccttactcccagccgactgtgcgccaagatgcttctaaacccatcacctgctgtcttcactcaaatgatttcagaacaggatttgcgaccaggtttatggggagattgaatcaacgattggtctcaaagacagtccattctttatatacatgtttagcatttttaccaacctcacatcatgtgtatatttgtgtatttgcacatggttgtgctgtcgaggacctggtgctgagaagagtctgttcacagccaaaattcttcccactgtcattcctaacctgggatttctagacacatcctgctgtgatgtaaacagaaatcacgaattcgctcactggatcaagttgttccactggtgtctaatacgctattgttgccggaggtgggttctgtgacgtgaagccatttcccatcattcaacagccagttacaattttctgtttaattaaattcatatttaaacaaaaa

SEQ ID NO: 180 - DNA, 8442 bp, Homo sapiens
  source 1..8442: mol_type = other DNA
gtccgcactctccgtccccgcggctggcgcaggacctcactcgagcggagcgcccacggggagcgggtcgcggggcggcggcggcgaggaggaggcgagaaggagttggaggaggaggaggaggaggcgagggcgagctagcccagcggggtcccggccgccccgcgggccaaagtcgagccctcccgcccgtgggcgagcgcgccagccgccccttccagaacagccgccgccacaaagaagaacggggggtgccgaggtccccatgacctcctaaagtggtgcggtccctgctgagtgcgctgcccgggccgtgacccgcgcccctgtgcgtccccgcgcgcctccgagcgcccctgtgcgccccggcccgcgccccgccggcatggacgtccatacccgctggaaagcgcgcagcgcgctccgcccgggcgccccgctgctgcccccgctgctgctgctgctgctgtgggcgccgcctccgagccgcgcagctcagccagcagatctcctgaaggttctagattttcacaacttgcctgatggaataacaaagacaacaggcttttgcgccacgcggcgatcttccaaaggcccggatgtcgcttacagagtcaccaaagacgcgcagctcagcgcacccaccaagcagctgtaccctgcgtctgcatttcccgaggacttctccatcctaacaactgtgaaagccaagaaaggcagccaggccttcctggtctccatctacaacgagcagggtatccagcagattgggctggagctgggccgctctcccgtcttcctctacgaggaccacacggggaagcctggcccggaagactaccccctcttccggggcatcaacctgtcagatggcaagtggcacagaattgctctcagcgtccacaagaaaaatgtcaccttgatcctcgactgtaaaaagaagaccaccaaattcctcgaccgcagcgaccaccccatgatcgacatcaatggcatcatcgtgtttggcacccggatcctggatgaggaggtgtttgagggtgacatccagcagctgctctttgtctcggaccaccgggcagcttatgattactgtgagcactacagccctgactgtgacaccgcagtacctgacaccccacagtcgcaggaccccaatccagatgaatattacacggaaggagacggcgagggtgagacctattactacgaatacccctactacgaagaccccgaagacctagggaaggagcccacccccagcaagaagcccgtggaagctgccaaagaaaccacagaggtccccgaggagctgaccccgacccccacggaagctgctcccatgcctgaaaccagtgaaggggctgggaaggaagaggacgtcggcatcggggactatgactacgtgcccagtgaggactactacacgccctcaccgtatgatgacctcacctatggcgagggggaggagaaccccgaccagcccacagacccaggcgctggggccgaaattcccaccagcaccgccgacacctccaactcctccaatccagctccgcctccaggggaaggtgcggatgacttggagggggagttcactgaggaaacgatccggaaccttgacgagaactactacgacccctactacgaccccaccagctccccgtcggagatcgggccgggaatgccggcgaaccaggataccatctatgaagggattggaggacctcggggcgagaaaggccaaaagggagaaccagcgattatcgagccgggcatgctcatcgagggcccgcctggcccagaaggccccgcgggtcttcccggacctccaggaaccatgggtcccactggccaagtcggggaccctggagaaaggggcccccctggacgcccaggccttcctggggccgatggcctgcccggtcctccaggaaccatgctcatgctgcccttccggtttggaggtggcggcgatgcgggctccaaaggccccatggtctcagcccaggagtcccaggcgcaagccattctccagcaggccaggttggcactgaggggaccagctggcccgatgggtctcacagggagacctggccctgtgggtccccctgggagcggaggtttgaagggcgagccgggagacgtggggcctcagggtcctcgaggtgtgcaaggcccgcctggtccggccgggaagcccggaagacggggtcgggctgggagtgatggagccagaggaatgcctggacaaactggccccaagggtgaccggggtttcgacggcctggctgggttgccaggcgagaagggccacaggggtgaccctggtccttccggcccaccaggacctccgggagacgatggagaaaggggtgacgacggagaagttgggcccagggggctgcctggggagcccgggccacgtggtctgcttgggccgaaggggcccccaggtcctcccggacctcccggtgtcacgggtatggacggccagccggggccaaaaggaaatgtgggtccccagggagagcctggccccccaggacagcagggtaatccaggcgcccagggtcttccaggcccccagggtgcaattggtcctccaggagaaaagggtcccttggggaaaccaggccttccaggaatgcccggtgctgacggacccccgggacaccctggcaaagaaggccctccaggagagaaaggaggtcagggtccacctggcccccagggtccgattggctacccaggtcctcgaggagtcaagggggccgatggcatccgtggtctgaagggcacaaagggcgagaagggtgaagacggctttcctgggtttaaaggagacatgggcatcaagggtgatcggggggagatcggcccacccggtcccaggggagaagatggccctgaaggcccaaagggtcgcggaggtcccaatggtgaccccggtcctctgggaccccctggggagaagggaaaactcggagtcccagggttaccagggtatccaggaagacaaggaccaaagggctctattggattccctggatttcctggcgccaatggagagaagggcggcagggggacccctggaaagccaggaccgcgggggcagcgaggcccaacgggtccgaggggtgaaagaggcccccggggcatcactgggaagcctggccccaagggcaactccggaggtgacggcccagctggccctcctggtgaacggggacccaatggaccccaaggacccacaggatttcctggaccaaagggcccccctggccctccaggcaaggatggactcccaggacaccctggacagagaggcgagactggtttccaaggcaagaccggccctccaggcccccccggcgtggtcggccctcagggtcccacgggagaaacgggcccaatgggtgagcgtggccaccctgggccccctggaccccccggtgaacaggggcttccgggccttgctggaaaagaagggacgaagggtgacccaggccctgcaggcctccctgggaaagatggccctccaggattacgtggtttccctggggaccgagggcttcctggtccagtgggagctcttggactgaaaggcaatgaagggccccctggcccaccaggccctgcgggatctccaggggagagaggtccagctggagccgctgggcccatcggaattccagggagacctgggccccagggacccccagggccggcaggagagaaaggggctcctggcgagaaaggcccacaaggcccagctggccgagacggtctccaggggcctgtggggctcccgggtccagctggccctgtgggtccccctggagaagacggagataagggagagatcggggagccggggcagaaaggaagcaagggggacaaaggagaacagggtcctcctgggcctacaggtcctcaaggccccatcggacagccaggcccctctggagctgacggcgagccggggcctcggggccagcagggccttttcgggcagaaaggtgatgaaggtcccagaggctttcctggaccccctgggccagtggggctgcagggtttgccaggacctccaggcgagaagggtgagacaggagacgtgggccagatgggccccccgggtccccctggcccccgaggaccctccggagctccaggtgctgatggcccacaaggtcccccaggtggaataggaaaccctggtgcagtgggagagaagggcgagcctggcgaagcaggtgagcctggccttccgggagaaggcggccccccgggacccaaaggagaaaggggagagaagggcgagtcaggcccttcaggtgctgccggaccccctggacccaaaggccctcccggagatgatggtcccaaaggcagccctggcccagtgggttttcctggagatcctggcccccccggagagcctggccccgcgggtcaagatggtccccctggtgacaaaggagatgatggtgaacccgggcagacgggatcccccggccctactggtgaaccaggtccatcggggcctccaggaaaaaggggtcccccaggccccgcaggccccgaaggcagacagggagagaaaggggccaagggagaagccggcttggaaggccctcctgggaagactggccccatcggcccccagggggcccctgggaagcccggaccggatggccttcgagggatccctggccctgtgggagaacaaggtctcccaggatccccaggcccggacggtccccccggccccatgggtcccccaggacttcccggcctcaaaggagattctggtcccaaaggtgaaaagggtcatccaggcctgatcgggctcatcggtcctccgggtgaacagggtgagaagggcgaccgtggtctccctggcccccagggctcctccggtcctaagggagaacagggtatcactggtccttctggcccgattgggcctcctgggccccctggcctgccgggtccgcctggtccaaaaggtgctaagggctcctcgggtccaactggcccgaagggtgaggcaggccacccaggacccccaggccccccgggccccccgggagaggtcatccagcccctgccaatccaggcatccaggacgcggcggaacatcgacgccagccagctgctggacgacgggaatggcgagaactacgtggactacgcggacggcatggaagagatcttcggctctctcaactctctgaagctggagattgagcagatgaaacggcccctgggcacgcagcagaaccccgcccgcacctgcaaggacctgcagctctgccaccccgacttcccagatggtgaatactgggtcgatcctaaccaaggatgctccagggattccttcaaggtttactgcaacttcacagccggggggtcgacatgcgtcttccctgacaagaagtccgaaggggccagaatcacttcttggcccaaagaaaacccgggctcctggttcagtgaattcaagcgtgggaaactgctctcctatgtggacgccgagggcaaccctgtgggtgtggtacagatgaccttcctgcggctgctgagcgcctctgcccaccagaacgtcacctaccactgctaccagtcagtggcctggcaggacgcagccacgggcagctacgacaaggccctccgcttcctgggctccaacgacgaggagatgtcctatgacaacaacccctacatccgcgccctggtggacggctgtgctaccaagaaaggctaccagaagacggttctggagatcgacacccccaaagtggagcaggtgcccatcgtggacatcatgttcaatgacttcggtgaagcgtcacagaaatttggatttgaagtggggccggcttgcttcatgggctaggagccgccgagcccgggctcccgagagcaacctcgtgacctcagcatgccattcgttcgtgagtgtcccgtgcacgtcctgaccctggacagtgaaggcttctccctcccctcccacctgacttcatctacgcctcggcaccacggggtgtgggaccccagcccggagagaacagagggaaggagccgcgcccccacctggagctgaatcacatgacctagctgcaccccagcgcctgggcccgccccacgctctgtccacacccacgcgccccgggagcggggccatgcctccagccccccagctcgcccgacccatcctgttcgtgaataggtctcaggggttgggggagggactgccagatttggacactatatttttttctaaattcaacttgaagatgtgtatttcccctgaccttcaaaaaatgttccaaggtaagcctcgtaaaggtcatcccaccatcaccaaagcctccgtttttaacaacctccaacacgatccatttagaggccaaatgtcattctgcaggtgccttcccgatggattaaaggtgcttatgtttttgtgagttttaagtaaatatttgtattgtattgttataaatgttaagtgtgcctggctttcaatcatgcacggaaacccagtctcagtcccacggacagaatgggcgaggcatggattctgggttgcagtaccgttctgattagaaataggaagtctccccacccccgccctggccaagaacgtgcaataaattggaagtttgccccggggcagcaagaatttatgctgccattgaaaagcaggtaccagtgccccttttcagacagtttttgattcgctctagactttttttttttttaatagggaaaaaatttgataattttcttttttctacatgcacttaagactaaaacacaggtttggattaattttatttgcttcctttttccgcttttcttcccgcagagcctgatgggagaatgtccagggcagggaaaccacattttttgtaggtgataactcaatgaaaattggtgcttattttttacacttctctcttgtggctctcttgtggtgctatctatctgttttaaggtctccttgaaggcgcactggggaccctggccatgcctcgttctccctgctttctttatcctgttattgcctccacagtctgttgccaaggactctaagatcaatgcacgtcactttcctttccactgggcaggatagccaagcacactccctcctgcgctctcccgccccggtgcgtccactcccgagggctgttatgaggactgggttgtgcctacttgatttgaaaacacacacaagcaataaaaagcctcttcctgcattgtctgtggtgtgaccatagcagattatatttggttcctgaatgtttgtggtgctaatttctgtgtttgttccaagccgttcagtcatgccatgcgctgcctcggtagatggagtaatgtacaatgaactccatgagtctctccagggctgcctgcagcacgtcttttccaagtagcctatttggattcccatctcaaatgtcctggatgcgagcgtcagcggctccagagctcggggcgggtgaggtcccctttggggaaccctttcctggccatcgaggtcggggggctgccgtctgtgggcaggaggacccgaggggcagccaggaaaggcgatctcttcactgtgaaaagttgcccgggtgcagcgccttttccttctaccatgggaaatgcaggctgggcccttggggtgagcctgcggggctctggtgctgtccccgacccccaccaccaccagaatgcagttccagcttaggaagccacaaacaagccacccaggaggaacaaaacaccgccagcgtggattttccaaatttccctggaaagtaagtctcgctcttgccaaagaaaagtctggcttggagagtctctggagcccaggatgccagcatgtgccaatgactgtcaccttcatctcttcaaaagaaaagccatagccgaggactgtcccgcgacccccgtggactgcgtctaggtcatgtgattctgttttcatttctcatcccatccaatttgtccttttctcctgtcattttcttcctctgtggtcccttcaaagttgttataatttgtactgaacttcaaaatgtgtcccgttctccccagaccactctagccacagtatattgcaataaaattacttcttatatttgcagaaattcttttggtgtaattttattttttcctctcaatatatataattggacaaacgctggcaaaaagaaaaaaatggtaagcaaaaaacccaagataaagtttcgaggacatcaggccttttgaaatacaatgtcaaatgacacattgtacggtttcaaaaaatccgctagacatgtcataagttttaactgtaatgcccaggaaaggatatcttaaaatattctaaacttgtgtaacaaaggaataattaactgtaatagtttttcaataaatcgagttgggtgtttccaccgtaaa

SEQ ID NO: 181 - DNA, 4260 bp, Homo sapiens
  source 1..4260: mol_type = other DNA
cgaggagcgagccagtgggggaggctgacatcaccacggcggcagccctttaaacccctcacccagccagcgccccatcctgtctgtccgaacccagacacaagtcttcactccttcctgcgagccctgaggaagccttgtgagtgcattggctggggcttggagggaagttgggctggagctggacaggagcagtgggtgcatttcaggcaggctctcctgaggtcccaggcgccagctccagctccctggctagggaaacccaccctctcagtcagcatgggggcccaagctccaggcagggtgggctggatcactagcgtcctggatctctctcagactgggcagccccgggctcattgaaatgccccggatgacttggctagtgcagaggaattgatggaaaccaccggggtgagagggaggctccccatctcagccagccacatccacaaggtgtgtgtaagggtgcaggcgccggccggttaggccaaggctctactgtctgttgcccctccaggagaacttccaaggagctttccccagacatggccaacaagggtccttcctatggcatgagccgcgaagtgcagtccaaaatcgagaagaagtatgacgaggagctggaggagcggctggtggagtggatcatagtgcagtgtggccctgatgtgggccgcccagaccgtgggcgcttgggcttccaggtctggctgaagaatggcgtgattctgagcaagctggtgaacagcctgtaccctgatggctccaagccggtgaaggtgcccgagaacccaccctccatggtcttcaagcagatggagcaggtggctcagttcctgaaggcggctgaggactatggggtcatcaagactgacatgttccagactgttgacctctttgaaggcaaagacatggcagcagtgcagaggaccctgatggctttgggcagcttggcagtgaccaagaatgatgggcactaccgtggagatcccaactggtttatgaagaaagcgcaggagcataagagggaattcacagagagccagctgcaggagggaaagcatgtcattggccttcagatgggcagcaacagaggggcctcccaggccggcatgacaggctacggacgacctcggcagatcatcagttagagcggagagggctagccctgagcccggccctcccccagctccttggctgcagccatcccgcttagcctgcctcacccacacccgtgtggtaccttcagccctggccaagctttgaggctctgtcactgagcaatggtaactgcacctgggcagctcctccctgtgcccccagcctcagcccaacttcttacccgaaagcatcactgccttggcccctccctcccggctgcccccatcacctctactgtctcctccctgggctaagcaggggagaagcgggctgggggtagcctggatgtgggccaagtccactgtcctccttggcggcaaaagcccattgaagaagaaccagcccagcctgccccctatcttgtcctggaatatttttggggttggaactcaaaaaaaaaaaaaaaaaatcaatcttttctcaggcctggctggcagagtttgatttgccctggtcacttttgttatggtttcagatctgcgcaggggacaggcaggcatctcgggcttgtatgtgttcctggctgccgtcctctgaaggacccttcatgtgccaagattccaggacagtcagattctttcagaggaaacaggccatagaacaggagagtgaatcttggggaccgaagggaaaaaggagccactcagcagcatgcagtggctttggggagacagaggaaaggagagattgcgagtggcagaattgtttggaaggtttttattttggaaaagctgtgcagaaaaaaattcaagaaaatgttgctgtgaaaagagaaaaacaaatcttaagcattaaaaaaaattcaaccaactagctcagaagaggggagaaggccaaggttgagagaagagtcacagcctgtcaggcagatagctggcctccggcgggaaggccatttccctgagcacttgcagaggaagacagggtggtggcaggactggagtggcagtggctcccaaggctctctcctccaacatgtgcatctgccatctgctctgcagtcctgccgcaggattccctagtgaagcagctcaggcctgggggagccgtgtgtatcccagctgtgccggcagcatcataccaatcgtgggggtggtgaaggagccaggggttcattcatggttggtttctgatcaggcatcttgggaatggataatggaggcagccgctttcaggacaggcatgtccaggggctcctcccagcctctaccccgaagtcctcttcccaagtgaccccagtgatgtttccattgagatgcgctcctggctatggcaggcaccttctcaacttatatgtgggaaggggtcccccatgcttgggggacctaggcagctggcttggcccaagagagatgaggatggaggccaaaccaaaggggggcgccaatcccctgtccaacaccttctcaccaaaagctcccgtttggctggaggcagaccctgtggctctgaccagacttctctggcagcaatcctccaccatttgtatcttaagaaggccctcaccctctttgagtggagtcagaggatgcctcagattccagatgtaagcatcagaactgctttctgtcaagagctccctaattttgggaaagaagagcctgtcccacactgtcaggccctgaggtcagcagatctgctcctccttcccgtgcggtacgtctaggtgctgatgagggcagtccagggcgctcttgttctgggacaggctccagtccccttgctccagcaggtctggggcaaggaggtcagaggtggtgggagggcccctgctctctgtttccacttcgtctggatccttgctgctgcaaagtggcactgattctagctctgtcccttcctccttggctttccggctccgatggggccagtggcagggtccactcctacaaacttgattggaagccacattcctctggctcaaatatacttccagcatgtagtaaacagtccagaagacggtgaaacagcctaccagcaccagggtctggggccgaggcaagcacctacgtgaggcactgtttcccgaagcctacagcctttctcagcccaggacaatgagctcagaaagtctttttccttctagggactgcctttttcacccaaactgttccctctgccccctcccccgacaaggcctgtcctgtaagagttaggctagcagggtaggagcaatgctatgaaaccactgctgggacccaggtcttcccagtccttcaccccgcaggtcaccctgagaaacacactaagaactccatcccccagacaggacagtcccatctacccgcagcttgagtcttggccctagtaccttgagggtgttgggggtgatggtgtaaccatcttcctcaggaattttcagccccggtgggcagggcagggcgaagtcatcgtgcaggtattttccactcatggcactctctaacagcctgtggagaagaaaagaggtgaggatgccccactgactatggttcctataccatctgccctcacccattttcctgccttacaggtgaagacagggaagccccacagcaaaaaggagaggcccagactgtgagttcccagcccggggctccatctaccggcttgacgctggaactgggaagcacagctggggttcagagggcgatgggctccctaggcggcccttcccctgtgactggccttccctctgacacatgcggctttacccacctttgtctgtccctgatgtctactgcactccacacagagccatatagggtcagctgccattgccggaggatgccgacctggaatgactcatcccctagggcagcaaggggcagctcgtgagccacgcccctctgtcagccatgccagacagcagaggccctgcccgccctgccacccaaaggggctcaggccacatggtctgctccaggagctgcctcactgtgtcccactgaccccaggttctgcagaagggcctcactgggtgcccttagggatggaaagggttgaaaggctgtactccaaagcagagtcttgcttttctctcccgtattttgggggttcagctgggattagaaaaaaatgtctttccaccaaattaaagaaagctttgaaaaccac

SEQ ID NO: 182 - DNA, 3156 bp, Homo sapiens
  source 1..3156: mol_type = other DNA
acagctgtgtttggctgcagggccaagagcgctgtcaagaagacccacacgcccccctccagcagctgaattcctgcagctcagcagccgccgccagagcaggacgaaccgccaatcgcaaggcacctctgagaacttcaggatgcagatgtctccagccctcacctgcctagtcctgggcctggcccttgtctttggtgaagggtctgctgtgcaccatcccccatcctacgtggcccacctggcctcagacttcggggtgagggtgtttcagcaggtggcgcaggcctccaaggaccgcaacgtggttttctcaccctatggggtggcctcggtgttggccatgctccagctgacaacaggaggagaaacccagcagcagattcaagcagctatgggattcaagattgatgacaagggcatggcccccgccctccggcatctgtacaaggagctcatggggccatggaacaaggatgagatcagcaccacagacgcgatcttcgtccagcgggatctgaagctggtccagggcttcatgccccacttcttcaggctgttccggagcacggtcaagcaagtggacttttcagaggtggagagagccagattcatcatcaatgactgggtgaagacacacacaaaaggtatgatcagcaacttgcttgggaaaggagccgtggaccagctgacacggctggtgctggtgaatgccctctacttcaacggccagtggaagactcccttccccgactccagcacccaccgccgcctcttccacaaatcagacggcagcactgtctctgtgcccatgatggctcagaccaacaagttcaactatactgagttcaccacgcccgatggccattactacgacatcctggaactgccctaccacggggacaccctcagcatgttcattgctgccccttatgaaaaagaggtgcctctctctgccctcaccaacattctgagtgcccagctcatcagccactggaaaggcaacatgaccaggctgccccgcctcctggttctgcccaagttctccctggagactgaagtcgacctcaggaagcccctagagaacctgggaatgaccgacatgttcagacagtttcaggctgacttcacgagtctttcagaccaagagcctctccacgtcgcgcaggcgctgcagaaagtgaagatcgaggtgaacgagagtggcacggtggcctcctcatccacagctgtcatagtctcagcccgcatggcccccgaggagatcatcatggacagacccttcctctttgtggtccggcacaaccccacaggaacagtccttttcatgggccaagtgatggaaccctgaccctggggaaagacgccttcatctgggacaaaactggagatgcatcgggaaagaagaaactccgaagaaaagaattttagtgttaatgactctttctgaaggaagagaagacatttgccttttgttaaaagatggtaaaccagatctgtctccaagaccttggcctctccttggaggacctttaggtcaaactccctagtctccacctgagaccctgggagagaagtttgaagcacaactcccttaaggtctccaaaccagacggtgacgcctgcgggaccatctggggcacctgcttccacccgtctctctgcccactcgggtctgcagacctggttcccactgaggccctttgcaggatggaactacggggcttacaggagcttttgtgtgcctggtagaaactatttctgttccagtcacattgccatcactcttgtactgcctgccaccgcggaggaggctggtgacaggccaaaggccagtggaagaaacaccctttcatctcagagtccactgtggcactggccacccctccccagtacaggggtgctgcaggtggcagagtgaatgtcccccatcatgtggcccaactctcctggcctggccatctccctccccagaaacagtgtgcatgggttattttggagtgtaggtgacttgtttactcattgaagcagatttctgcttccttttatttttataggaatagaggaagaaatgtcagatgcgtgcccagctcttcaccccccaatctcttggtggggaggggtgtacctaaatatttatcatatccttgcccttgagtgcttgttagagagaaagagaactactaaggaaaataatattatttaaactcgctcctagtgtttctttgtggtctgtgtcaccgtatctcaggaagtccagccacttgactggcacacacccctccggacatccagcgtgacggagcccacactgccaccttgtggccgcctgagaccctcgcgccccccgcgcccctctttttccccttgatggaaattgaccatacaatttcatcctccttcaggggatcaaaaggacggagtggggggacagagactcagatgaggacagagtggtttccaatgtgttcaatagatttaggagcagaaatgcaaggggctgcatgacctaccaggacagaactttccccaattacagggtgactcacagccgcattggtgactcacttcaatgtgtcatttccggctgctgtgtgtgagcagtggacacgtgaggggggggtgggtgagagagacaggcagctcggattcaactaccttagataatatttctgaaaacctaccagccagagggtagggcacaaagatggatgtaatgcactttgggaggccaaggcgggaggattgcttgagcccaggagttcaagaccagcctgggcaacataccaagacccccgtctctttaaaaatatatatattttaaatatacttaaatatatatttctaatatctttaaatatatatatatattttaaagaccaatttatgggagaattgcacacagatgtgaaatgaatgtaatctaatagaagcctaatcagcccaccatgttctccactgaaaaatcctctttctttggggtttttctttctttcttttttgattttgcactggacggtgacgtcagccatgtacaggatccacaggggtggtgtcaaatgctattgaaattgtgttgaattgtatgctttttcacttttgataaataaacatgtaaaaatgtttcaaaaaaataataaaataaataaatacgaa

SEQ ID NO: 183 - DNA, 5490 bp, Homo sapiens
  source 1..5490: mol_type = other DNA
ggctgagttttatgacgggcccggtgctgaagggcagggaacaacttgatggtgctactttgaactgcttttcttttctcctttttgcacaaagagtctcatgtctgatatttagacatgatgagctttgtgcaaaaggggagctggctacttctcgctctgcttcatcccactattattttggcacaacaggaagctgttgaaggaggatgttcccatcttggtcagtcctatgcggatagagatgtctggaagccagaaccatgccaaatatgtgtctgtgactcaggatccgttctctgcgatgacataatatgtgacgatcaagaattagactgccccaacccagaaattccatttggagaatgttgtgcagtttgcccacagcctccaactgctcctactcgccctcctaatggtcaaggacctcaaggccccaagggagatccaggccctcctggtattcctgggagaaatggtgaccctggtattccaggacaaccagggtcccctggttctcctggcccccctggaatctgtgaatcatgccctactggtcctcagaactattctccccagtatgattcatatgatgtcaagtctggagtagcagtaggaggactcgcaggctatcctggaccagctggccccccaggccctcccggtccccctggtacatctggtcatcctggttcccctggatctccaggataccaaggaccccctggtgaacctgggcaagctggtccttcaggccctccaggacctcctggtgctataggtccatctggtcctgctggaaaagatggagaatcaggtagacccggacgacctggagagcgaggattgcctggacctccaggtatcaaaggtccagctgggatacctggattccctggtatgaaaggacacagaggcttcgatggacgaaatggagaaaagggtgaaacaggtgctcctggattaaagggtgaaaatggtcttccaggcgaaaatggagctcctggacccatgggtccaagaggggctcctggtgagcgaggacggccaggacttcctggggctgcaggtgctcggggtaatgacggtgctcgaggcagtgatggtcaaccaggccctcctggtcctcctggaactgccggattccctggatcccctggtgctaagggtgaagttggacctgcagggtctcctggttcaaatggtgcccctggacaaagaggagaacctggacctcagggacacgctggtgctcaaggtcctcctggccctcctgggattaatggtagtcctggtggtaaaggcgaaatgggtcccgctggcattcctggagctcctggactgatgggagcccggggtcctccaggaccagccggtgctaatggtgctcctggactgcgaggtggtgcaggtgagcctggtaagaatggtgccaaaggagagcccggaccacgtggtgaacgcggtgaggctggtattccaggtgttccaggagctaaaggcgaagatggcaaggatggatcacctggagaacctggtgcaaatgggcttccaggagctgcaggagaaaggggtgcccctgggttccgaggacctgctggaccaaatggcatcccaggagaaaagggtcctgctggagagcgtggtgctccaggccctgcagggcccagaggagctgctggagaacctggcagagatggcgtccctggaggtccaggaatgaggggcatgcccggaagtccaggaggaccaggaagtgatgggaaaccagggcctcccggaagtcaaggagaaagtggtcgaccaggtcctcctgggccatctggtccccgaggtcagcctggtgtcatgggcttccccggtcctaaaggaaatgatggtgctcctggtaagaatggagaacgaggtggccctggaggacctggccctcagggtcctcctggaaagaatggtgaaactggacctcagggacccccagggcctactgggcctggtggtgacaaaggagacacaggaccccctggtccacaaggattacaaggcttgcctggtacaggtggtcctccaggagaaaatggaaaacctggggaaccaggtccaaagggtgatgccggtgcacctggagctccaggaggcaagggtgatgctggtgcccctggtgaacgtggacctcctggattggcaggggccccaggacttagaggtggagctggtccccctggtcccgaaggaggaaagggtgctgctggtcctcctgggccacctggtgctgctggtactcctggtctgcaaggaatgcctggagaaagaggaggtcttggaagtcctggtccaaagggtgacaagggtgaaccaggcggtccaggtgctgatggtgtcccagggaaagatggcccaaggggtcctactggtcctattggtcctcctggcccagctggccagcctggagataagggtgaaggtggtgcccccggacttccaggtatagctggacctcgtggtagccctggtgagagaggtgaaactggccctccaggacctgctggtttccctggtgctcctggacagaatggtgaacctggtggtaaaggagaaagaggggctccgggtgagaaaggtgaaggaggccctcctggagttgcaggaccccctggaggttctggacctgctggtcctcctggtccccaaggtgtcaaaggtgaacgtggcagtcctggtggacctggtgctgctggcttccctggtgctcgtggtcttcctggtcctcctggtagtaatggtaacccaggacccccaggtcccagcggttctccaggcaaggatgggcccccaggtcctgcgggtaacactggtgctcctggcagccctggagtgtctggaccaaaaggtgatgctggccaaccaggagagaagggatcgcctggtgcccagggcccaccaggagctccaggcccacttgggattgctgggatcactggagcacggggtcttgcaggaccaccaggcatgccaggtcctaggggaagccctggccctcagggtgtcaagggtgaaagtgggaaaccaggagctaacggtctcagtggagaacgtggtccccctggaccccagggtcttcctggtctggctggtacagctggtgaacctggaagagatggaaaccctggatcagatggtcttccaggccgagatggatctcctggtggcaagggtgatcgtggtgaaaatggctctcctggtgcccctggcgctcctggtcatccaggcccacctggtcctgtcggtccagctggaaagagtggtgacagaggagaaagtggccctgctggccctgctggtgctcccggtcctgctggttcccgaggtgctcctggtcctcaaggcccacgtggtgacaaaggtgaaacaggtgaacgtggagctgctggcatcaaaggacatcgaggattccctggtaatccaggtgccccaggttctccaggccctgctggtcagcagggtgcaatcggcagtccaggacctgcaggccccagaggacctgttggacccagtggacctcctggcaaagatggaaccagtggacatccaggtcccattggaccaccagggcctcgaggtaacagaggtgaaagaggatctgagggctccccaggccacccagggcaaccaggccctcctggacctcctggtgcccctggtccttgctgtggtggtgttggagccgctgccattgctgggattggaggtgaaaaagctggcggttttgccccgtattatggagatgaaccaatggatttcaaaatcaacaccgatgagattatgacttcactcaagtctgttaatggacaaatagaaagcctcattagtcctgatggttctcgtaaaaaccccgctagaaactgcagagacctgaaattctgccatcctgaactcaagagtggagaatactgggttgaccctaaccaaggatgcaaattggatgctatcaaggtattctgtaatatggaaactggggaaacatgcataagtgccaatcctttgaatgttccacggaaacactggtggacagattctagtgctgagaagaaacacgtttggtttggagagtccatggatggtggttttcagtttagctacggcaatcctgaacttcctgaagatgtccttgatgtgcatctggcattccttcgacttctctccagccgagcttcccagaacatcacatatcactgcaaaaatagcattgcatacatggatcaggccagtggaaatgtaaagaaggccctgaagctgatggggtcaaatgaaggtgaattcaaggctgaaggaaatagcaaattcacctacacagttctggaggatggttgcacgaaacacactggggaatggagcaaaacagtctttgaatatcgaacacgcaaggctgtgagactacctattgtagatattgcaccctatgacattggtggtcctgatcaagaatttggtgtggacgttggccctgtttgctttttataaaccaaactctatctgaaatcccaacaaaaaaaatttaactccatatgtgttcctcttgttctaatcttgtcaaccagtgcaagtgaccgacaaaattccagttatttatttccaaaatgtttggaaacagtataatttgacaaagaaaaatgatacttctctttttttgctgttccaccaaatacaattcaaatgctttttgttttatttttttaccaattccaatttcaaaatgtctcaatggtgctataataaataaacttcaacactctttatgataacaacactgtgttatattctttgaatcctagcccatctgcagagcaatgactgtgctcaccagtaaaagataacctttctttctgaaatagtcaaatacgaaattagaaaagccctccctattttaactacctcaactggtcagaaacacagattgtattctatgagtcccagaagatgaaaaaaattttatacgttgataaaacttataaatttcattgattaatctcctggaagattggtttaaaaagaaaagtgtaatgcaagaatttaaagaaatatttttaaagccacaattattttaatattggatatcaactgcttgtaaaggtgctcctcttttttcttgtcattgctggtcaagattactaatatttgggaaggctttaaagacgcatgttatggtgctaatgtactttcacttttaaactctagatcagaattgttgacttgcattcagaacataaatgcacaaaatctgtacatgtctcccatcagaaagattcattggcatgccacaggggattctcctccttcatcctgtaaaggtcaacaataaaaaccaaattatggggctgcttttgtcacactagcatagagaatgtgttgaaatttaactttgtaagcttgtatgtggttgttgatcttttttttccttacagacacccataataaaatatcatattaaaattc

SEQ ID NO: 184 - protein, 450 aa, synthetic construct
  REGION 1..450: note = Description of Artificial Sequence: Synthetic polypeptide
EVQLVESGGGVVQPGRSLRLSCAASGFTFSSYGMHWVRQAPGKELEWVAVISYDGSIKYYADSVKGRFTISRDNSKNTLYLQMNSLRAEDTAVYYCARTGEYSGYDTDPQYSWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKPTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 185 - protein, 213 aa, synthetic construct
  REGION 1..213: note = Description of Artificial Sequence: Synthetic polypeptide
EIVLTQSPSSLSASVGDRVTITCRASQGIGDDLGWYQQKPGKAPILLIYGTSTLQSGVPSRFSGSGSGTDFTLTINSLQPEDFATYYCLQDSNYPLTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSPVTKSFNRGEC

SEQ ID NO: 186 - protein, 219 aa, synthetic construct
  REGION 1..219: note = Description of Artificial Sequence: Synthetic polypeptide
EIVLTQSPGTLSLSPGERATLSCQSSQSVYNNNYLSWFQQKPGQAPRLLIYGASTLTSGIPDRFSGSGSGTDFTLTISRLEPEDFAVYYCAGGYSGSSDKYAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 187 - protein, 214 aa, synthetic construct [sequence omitted]

SEQ ID NO: 188 - protein, 448 aa, synthetic construct
  REGION 1..448: note = Description of Artificial Sequence: Synthetic polypeptide
  MOD_RES 1: note = Predominantly pyroglutamic acid (pE)
EVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG